(19) United States
(12) Patent Application Publication
(10) Pub. No.: US 2024/0024489 A1
(43) Pub. Date: Jan. 25, 2024

(54) PROTECTED DISACCHARIDES, THEIR PROCESS OF PREPARATION AND THEIR USE IN THE SYNTHESIS OF ZWITTERIONIC OLIGOSACCHARIDES, AND CONJUGATES THEREOF

(71) Applicant: INSTITUT PASTEUR, Paris (FR)

(72) Inventors: Laurence MULARD, Paris (FR); Debashis DHARA, Paris (FR); Helene PFISTER, Paris (FR); Julie PAOLETTI, Paris (FR); Armelle PHALIPON, Paris (FR); Catherine GUERREIRO-INVERNO, Paris (FR)

(21) Appl. No.: 18/252,787

(22) PCT Filed: Nov. 22, 2021
(86) PCT No.: PCT/EP2021/082559

§ 371 Date: May 12, 2023

(30) Foreign Application Priority Data
Nov. 20, 2020 (EP) 20306422.5

(51) Int. Cl. A61K 47/54; A61K 39/112; A61P 31/04

(52) U.S. Cl. CPC A61K 47/549; A61K 39/0283; A61P 31/04; A61K 2039/6087

(57) ABSTRACT

The present invention provides zwitterionic oligosaccharides, in particular fragments of the surface polysaccharides from Shigella sonnei and Shigella sonnei conjugates comprising them. The present invention also provides protected disaccharides, their process of preparation and their use in the synthesis of zwitterionic oligosaccharides, and conjugates thereof, the disaccharide repeating unit of Shigella sonnei being: (I)

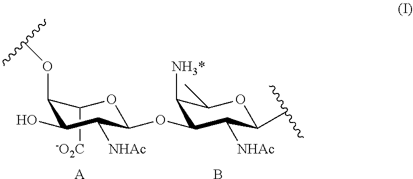

The present invention provides protected disaccharides, their process of preparation and their use in the synthesis of zwitterionic oligosaccharides, and conjugates thereof. The present invention also provides zwitterionic oligosaccharides, in particular fragments of the surface polysaccharides from Shigella sonnei, and Shigella sonnei conjugates comprising them.

Diarrheal diseases are a major public health burden worldwide and the second leading cause of death in children under 5 years of age. Recent studies have identified Shigella as one of the top agents causing moderate-to-severe diarrhea in this population. Still, the global burden of shigellosis is thought to be underestimated and the emergence of multidrug-resistant strains goes against antibiotic treatment as being the sole answer to Shigella burden. Fighting shigellosis by means of vaccines was recommended decades ago by WHO and vaccination is still viewed as a valuable preventive intervention. However, no broadly licensed Shigella vaccine is available despite a diversity of vaccine candidates tested in clinical trials.

Shigella sonnei, as a single serotype, causes an estimated 25% of all shigellosis episodes. It is the second most common Shigella species causing disease in low and middle income countries and the predominant species in high income and transitional countries. High incidence in traveler's diarrhea and increasing antibiotic resistance also contribute to concern for this Gram negative enteroinvasive bacterium. Evidence point to S. sonnei surface polysaccharides as being the major protective antigens against reinfection, and among the many strategies under investigation toward a S. sonnei vaccine, polysaccharide conjugates have emerged as a promising route. Otherwise, exploring the feasibility of using synthetic carbohydrate haptens as surrogates of the S. sonnei natural polysaccharide antigens is envisioned as a promising alternative.

The repeating unit from the S. sonnei O—Ag is a unique zwitterionic polysaccharide (ZPS) of following formula [4)-α-L-AltpNAcA-(1→3)-β-D-FucpNAc4N-(1→]:

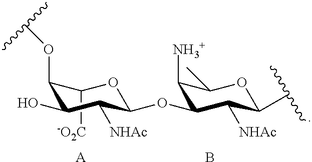

S. sonnei is to the inventors' knowledge also surrounded by a capsular polysaccharide (CPS). As recently disclosed, the two S. sonnei surface polysaccharides display the same zwitterionic repeating unit.

As for other ZPSs, the zwitterionic character of the surface polysaccharides from S. sonnei stems from adjacent monosaccharide units harboring alternating charges within the repeating unit. But to the inventors' knowledge, the S. sonnei ZPSs are the sole as of to date featuring a disaccharide repeating unit. The latter is made of two uncommon amino sugars, a 2-acetamido-2-deoxy-L-altruronic acid (L-AltpNAcA, A) and a 2-acetamido-4-amino-2,4,6-trideoxy-D-galactopyranose (D-FucpNAc4N, AAT, B) 1,2-trans-linked to one another.

Despite being an unusual component within the whole glycome, AAT has been identified in several other bacterial ZPSs, most often as an α-linked residue as exemplified in the CPS from Streptococcus pneumoniae serotype 1 (Sp1) and Bacteroides fragilis (PS A1). It was less frequently found in its β-form as present in S. sonnei and Plesiomonas shigelloides O17, which expresses an O—Ag identical to that of S. sonnei, and more recently identified in the LPS from Providencia alcalifaciens O22, another cause of diarrheal disease, and in the lipoteichoic acid of Streptococcus oralis Uo5. Owing to their characteristic immunomodulatory properties, ZPSs—especially Sp1 and PS A1—and synthetic fragments thereof have attracted a lot of interest in recent years whether aiming at developing vaccine haptens or for use as vaccine carrier. In that context, AAT has qualified as an attractive synthetic target.

In contrast, L-AltpNAcA is barely encountered, being to the inventors' knowledge originally reported as a key component of the S. sonnei and P. shigelloides O17 ZPSs. Besides its exceptional zwitterionic nature, a distinctive feature of the S. sonnei O—Ag is the occurrence of three amino groups, two of which present as acetamide, within a disaccharide repeat.

However, despite all the interest offered by compounds incorporating the AB disaccharide, their synthesis is made particularly difficult by the presence, in addition to the carboxylic acid group, of said three amino groups in different forms. Furthermore, these groups make a site-selective conjugation particularly difficult to achieve, in particular in a multiple fashion on a carrier and/or solid support.

Therefore, it is an object of the present invention to provide versatile core precursors, able to yield a great number of oligosaccharides in a highly efficient divergent manner.

Another aim of the invention is to provide core precursors and oligo- and polysaccharides that enable a site-selective conjugation on said oligo- and polysaccharides (i.e. that implies a single function on said oligo- and polysaccharides), to a carrier. Conjugation methods that are orthogonal to the functions naturally occurring on the target oligo- and polysaccharides (NH₂, CO₂, secondary OH, vicinal aminoalcohol, vicinal diol . . . ) are thus possible thanks to the core precursors and oligo- and polysaccharides of the invention.

Another aim of the present invention is to provide a way to a large variety of selected targets, in terms of oligo-, polysaccharides and conjugates thereof, in the context of vaccine development against S. sonnei related diseases, and also for the development of diagnostic tools.

Another aim of the present invention is to provide core precursors and intermediate compounds that bear finely tuned protective groups, which enable:
Efficient homologation to [AB]ₙ, B[AB]ₙ, [AB]ₙA and B[AB]ₙA sequences, even long ones, by providing precursors that can easily by converted into either a donor or an acceptor;
A highly efficient deprotection of all the protective groups in only one or two steps;
An easy coupling method to reactive residues or target compounds.

Thus, in a first object, the present invention provides a conjugate comprising an oligo- or polysaccharide selected from the group consisting of:

(B)ₓ-(A-B)ₙ-(A)_{y}, and

(A)ₓ-(B-A)ₙ-(B)_{y},

wherein:
x is 0 or 1,
y is 0 or 1,
n ranges from 1 to 50, in particular from 1 or 2 to 10, more particularly from 1 or 2 to 4 or from 3 to 8, n being notably 1, 2, 3, 4, 5, 6, 7, 8, 9 or 10,
A is 4)-α-L-AltpNAcA-(1→,
B is 3)-β-D-FucpNAc4N-(1→,
or a pharmaceutically acceptable salt thereof,
said oligo- or polysaccharide being bound to a carrier, in particular covalently bound to a carrier.
4)-α-L-AltpNAcA-(1→ refers in particular to:

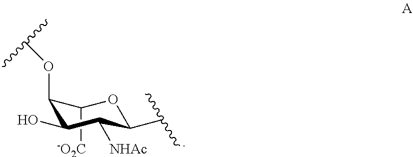

3)-β-D-FucpNAc4N-(1→ refers in particular to:

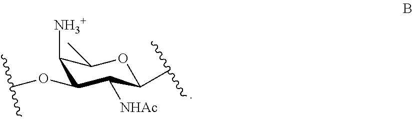

In a particular embodiment, x+y=1.

In another particular embodiment, x=y=0.

In a particular embodiment, n ranges from 1 or 2 to 10, more particularly from 1 or 2 to 4 or from 3 to 8, n being notably 1, 2, 3, 4, 5, 6, 7, 8, 9 or 10.

The term “conjugate” refers in particular to an oligo- or polysaccharide linked covalently to a carrier.

In a particular embodiment, the oligo- or polysaccharide is bound to the carrier via the reducing end of said oligo- or polysaccharide. Such a conjugation is thus site-selective and corresponds to a conjugate wherein the carrier is attached to the oligo- or polysaccharide via a single anchoring point.

In a particular embodiment, the oligo- or polysaccharide is bound to the carrier via the non-reducing end of said oligo- or polysaccharide. Such a conjugation is also site-selective and corresponds to a conjugate wherein the carrier is attached to the oligo- or polysaccharide via a single anchoring point. More particularly, the oligo- or polysaccharide is bound to the carrier via the non-reducing end of a B residue of said oligo- or polysaccharide, for example of formula (B)ₓ-(A-B)ₙ-(A)_{y}, wherein x is 1.

The oligo- or polysaccharide can be covalently bound to the carrier with or without a linking molecule or spacer.

The linking molecule or spacer does not contain any carbohydrate residue; thus, it is neither a carbohydrate residue nor an oligosaccharide- or a polysaccharide compound. The oligo- or polysaccharide is preferably conjugated to a carrier using a linking molecule. A linker or crosslinking agent, as used in the present invention, is preferably a small molecule, linear or not, having a molecular weight of approximately <500 daltons and is non-pyrogenic and non-toxic in the final product form, in particular in the framework of an in vitro use, or when the final product is an immunogenic composition for use in vaccination.

Advantageously, in addition to ensuring product homogeneity and avoiding microbial contamination, the use of synthetic oligo- or polysaccharides is fully compatible with their site selective attachment onto the carrier, thus opening the way to a controlled and robust conjugation process. On the one hand, the uncontrolled masking of epitopes important for protection is avoided, and on the other hand it becomes possible to eliminate side effects generated from neoepitopes possibly formed during conjugation.

Another advantage of the use of synthetic oligo- or polysaccharides is that they may be grafted in larger molar amounts than large heterogeneous bacterial polysaccharides. One can also identify the minimal and sufficient structures to play the desired role i.e. antigen or immunogenic (if conjugated).

Covalent linkage of synthetic oligo- and polysaccharides to proteins is known in the art and may for example be achieved by targeting the F-amines of lysines, the carboxylic groups of aspartic/glutamic acids, the sulfhydryls of cysteines, or tyrosines. A reactive group, for example an amine, can also be introduced at the oligosaccharide reducing termini, directly or via a linker, to be used finally for insertion of a bifunctional linker for conjugation to the carrier.

For example, the oligo- or polysaccharide may be conjugated to the carrier through the reaction between a maleimido or haloacetyl group, in particular bromoacetyl group, bound to the oligo- or polysaccharide, in particular via a linker, and a thiol or a NH₂ group bound to the carrier, in particular via a linker; or through the reaction between a maleimido or haloacetyl group, in particular bromoacetyl group, bound to the carrier, in particular via a linker, and a thiol or a NH₂ group bound to the oligo- or polysaccharide, in particular via a linker.

To conjugate with a linker or crosslinking agent, either or both of the oligo- or polysaccharide and the carrier may be covalently bound to one or more linkers first. The linkers or crosslinking agents are homobifunctional or heterobifunctional molecules, e.g., adipic dihydrazide, ethylenediamine, cystamine, N-succinimidyl 3-(2-pyridyldithio)propionate (SPDP), N-acetyl-DL-homocysteine thiolactone, N′-succinimidyl-[N-(2-iodoacetyl)-β-alanyl]propionate (SIAP), 3,3′-dithiodipropionic acid, squarates and their derivatives, and the like.

According to the type of linkage between the oligo- or polysaccharide and the carrier, there is the possibility of preparing a conjugate wherein the ratio of the oligo- or polysaccharide versus the carrier can in particular vary between 1:1 and 500:1, notably between 1:1 and 200:1. More particularly, this ratio is comprised between 1:1 and 30:1, preferably between 5:1 and 25:1, more preferably between 8:1 and 30:1, or between 5:1 and 20:1, notably when the carrier is tetanus toxoid or a fragment thereof.

A carrier can be a natural, modified-natural, synthetic, semi-synthetic or recombinant material containing one or more functional groups, for example primary and/or secondary amino groups, azido groups, thiol, alkynyl, alkenyl, or carboxyl group. The carrier can be water soluble or insoluble. Carriers that fulfil these criteria are well-known to those of ordinary skill in the art.

Suitable carriers according to the present invention notably include proteins, peptides, lipopeptides, zwitterionic polysaccharides, lipid aggregates (such as oil droplets or liposomes), inactivated virus particles, nanoparticles, in particular gold nanoparticles (reference is for example made to Bioorganic Chemistry 99 (2020) 103815, or Nanomedicine 2012, 7:651-662), virus-like particles, for example bacteriophage Qβ (VLPs Methods Enzymol 2017; 597:359-376) and Generalized Modules for Membrane Antigens (GMMA; reference is for example made to: Vaccines 2020, 8, 540; Vaccines (Basel). 2020 Apr. 3; 8(2):160).

In a particular embodiment, the carrier is a protein.

In this case, the term “carrier” refers in particular to a protein to which the oligo- or polysaccharide is coupled or attached or conjugated, typically for the purpose of enhancing or facilitating detection of the antigen by the immune system. Oligosaccharides are T-independent antigens that are poorly immunogenic and do not lead to long-term protective immune responses. Conjugation of the oligosaccharide antigen to a protein carrier changes the context in which immune effector cells respond to oligosaccharides. The term carrier protein is intended to cover both small peptides and large polypeptides (>10 kDa). In a particular embodiment, the carrier is an immunocarrier.

Immunocarriers are carriers chosen to increase the immunogenicity of the oligo- or polysaccharide and/or to raise antibodies against the carrier which are medically beneficial.

Suitable immunocarriers according to the present invention notably include proteins, glycosphingolipids, peptides, lipopeptides, lipid aggregates containing T-helper peptides (at least one), inactivated virus particles, nanoparticles, in particular gold nanoparticles (as for example described in NPJ Vaccines 2020, 5(1), 8), and Generalized Modules for Membrane Antigens (GMMA).

In a particular embodiment, the conjugate of the invention is covalently bound to a protein or a peptide comprising at least one T-helper epitope.

According to an advantageous embodiment, the glycoconjugate of the invention is covalently bound to a protein or a peptide comprising at least one T-helper epitope, for use as a vaccine against S. sonnei infection and/or infection caused by pathogens featuring cross-reactive carbohydrate antigens, for example a Plesiomonas shigelloides infection, notably a P. shigelloides O17 infection.

Protein carriers known to have potent T-helper epitopes, include but are not limited to bacterial toxoids such as tetanus, diphtheria and cholera toxoids, Staphylococcus exotoxin or toxoid, Pseudomonas aeruginosa Exotoxin A and recombinantly produced, genetically detoxified variants thereof, outer membrane proteins (OMPs) of Neisseria meningitidis and Shigella proteins. The recombinantly-produced, non-toxic mutant strains of P. aeruginosa Exotoxin A (rEPA) are described and used in polysaccharide-protein conjugate vaccines (Infect Immun 1993, 61, 1023-1032). The CMR197 carrier is a well characterized non-toxic diphtheria toxin mutant that is useful in glycoconjugate vaccine preparations intended for human use (a) Adv Exp Med Biol 1989, 251, 175-180; b) Vaccine 1992, 10, 691-698). Other exemplary protein carriers include the Fragment C of tetanus toxin (WO 2005/000346, WO 2005/000346). Also CRM9 carrier has been disclosed for human immunisation (Pediatr Infect Dis J 2003, 22, 701-706).

Useful carrier proteins include bacterial toxins or toxoids, such as diphtheria toxoid or tetanus toxoid. Fragments of toxins or toxoids can also be used e.g. fragment C of tetanus toxoid (commercially available). The CRM 197 mutant of diphtheria toxin is a particularly useful with the invention. Other suitable carrier proteins include the Neisseria meningitidis outer membrane protein, synthetic peptides, heat shock proteins, pertussis proteins, cytokines, lymphokines, hormones, growth factors, human serum albumin (preferably recombinant) in particular for diagnostic aspects, universal CD4+ cell epitopes, in particular artificial proteins comprising multiple human CD4+ T cell epitopes from various pathogen-derived antigens such as N19 or tetanus toxoid (Cancer Immunol Immunother. 2016, 65(3), 315-25), protein D from Haemophilus influenzae, pneumococcal surface protein PspA, pneumolysin, iron-uptake proteins, toxin A or B from Clostridium difficile, recombinant P. aeruginosa exoprotein A (rEPA), a GBS protein, and the like, as for example described in Micoli et al. (Molecules 2018, 23(6), 1451).

Particularly suitable carrier proteins include CRM 197, tetanus toxoid (TT), tetanus toxoid fragment C, protein D, non-toxic mutants of tetanus toxin and diphtheria toxoid (DT). Other suitable carrier proteins include protein antigens GBS80, GBS67 and GBS59 from Streptococcus agalactiae and fusion proteins, for example, GBS59(6xD3) disclosed in WO2011/121576 and GBS59(6xD3)-1523 disclosed in EP14179945.2. The use as other suitable carrier proteins of proteins antigens that are common to several Shigella serotypes such as IpaD, IpaB, MxiH and all their possible combinations may also be advantageous. Another carrier could be genetically modified OMVs (GMMA), for example those developed by the pharmaceutical industry. Synthetic peptides bearing immunodominant T-helper cell epitopes can also act as carriers in polysaccharide and oligosaccharide conjugates. The peptide carriers include polypeptides containing multiple T-helper epitopes addressing the extensive polymorphism of HLA molecules (Pediatrics 1993, 92, 827-832), and universal T-helper epitopes compatible with human use. Exemplary T-helper epitopes, include but are not limited to natural epitopes characterized from tetanus toxoid (J Immunol 1992, 149, 717-721), and non-natural epitopes or engineered epitopes such as the pan HLA DR-binding epitope PADRE (Immunity 1994, 1, 751-761; Vaccine 2004, 22(19), 2362-7).

Carriers also include lipopeptides, for example Pam(3)CAG (Vaccine 2009, 27(39), 5419-26), as an adjuvant.

Carriers also include zwitterionic polysaccharides, as for example described in Chem Sci 2020, 11(48), 13052-13059.

In a particular embodiment of the present invention, the immunocarrier is selected among a protein or a peptide comprising at least one T-helper epitope, or a derivative thereof.

By derivative is in particular meant here a peptide comprising at least one T-helper epitope, which is thus longer than the corresponding T-helper epitope, for example for solubility reasons.

In a particular aspect, the immunocarrier is the peptide PADRE.

In a particular embodiment of the present invention, the immunocarrier is tetanus toxoid (TT) or a fragment thereof, in particular fragment He of TT.

In another particular embodiment of the present invention, the immunocarrier is CRM 197.

In another particular embodiment of the present invention, the immunocarrier is diphtheria toxoid, protein D, in particular Haemophilus influenzae b protein D, OMV, in particular Neisseria meningitidis OMV, PADRE, recombinant P. aeruginosa exoprotein A (rEPA).

The term “toxoid” as used herein refers to a bacterial toxin (usually an exotoxin), whose toxicity has been inactivated or suppressed either by chemical (formalin) or heat treatment, while other properties, typically T-helper properties and/or immunogenicity, are maintained. A mutated toxoid as used herein is a recombinant bacterial toxin, which has been amended to be less toxic or even non-toxic by amending the wild-type amino acid sequence. Such a mutation could be a substitution of one or more amino acids. Such a mutated toxoid presents on its surface a functionality that can react with the functional group of the interconnecting molecule to provide a modified toxoid. Said functionality is known to the person skilled in the art and includes, but is not restricted to the primary amino functionality of a lysine residue that can react with activated esters, an isocyanate group or an aldehyde in presence of a reducing agent, to the carboxylate functionality of a glutamate or aspartate residue that can be activated by carbodiimides or to the thiol functionality of a cysteine residue.

Activated esters include, but are not restricted to N-(y-maleimidobutyryloxy) succinimide ester (GMBS), N-(y-maleimidobutyryloxy) sulfosuccinimide ester (sulfo-GMBS), succinimidyl (4-iodoacetyl) aminobenzoate (sulfo-SIAB), succinimidyl-3-(bromoacetamido)propionate (SBAP), disuccinimidyl glutarate (DSG), disuccinimidyl adipate (DSA), 2-pyridyldithiol-tetraoxatetradecane-N-hydroxysuccinimide (PEG-4-SPDP), bis-(4-nitrophenyl) adipate and bis-(4-nitrophenyl) succinate. Preferred activated esters are for example N-(y-maleimidobutyryloxy) succinimide ester (GMBS), N-(y-maleimidobutyryloxy) sulfosuccinimide ester (sulfo-GMBS), succinimidyl (4-iodoacetyl) aminobenzoate (sulfo-SIAB), succinimidyl-3-(bromoacetamido)propionate (SBAP).

The cysteine residue on the carrier protein can be converted to the corresponding dehydroalanine that can be further reacted with a suitable interconnecting molecule to provide modified carrier protein having on their surface the functional group of the interconnecting molecule.

For example, the inventive saccharides described herein are conjugated to the non-toxic mutated diphtheria toxin CRM₁₉₇ presenting as a functionality a primary amine functionality of a lysine residue.

CRM₁₉₇ like wild-type diphtheria toxin is a single polypeptide chain of 535 amino acids (58 kD) consisting of two subunits linked by disulfide bridges having a single amino acid substitution of glutamic acid for glycine. It is utilized as a carrier protein in a number of approved conjugate vaccines for diseases such as Prevnar.

It is especially preferred that inventive saccharides described herein are conjugated to tetanus toxoid (TT) or a fragment thereof presenting as a functionality a primary amine functionality of a lysine residue.

It is especially preferred that inventive saccharides described herein are conjugated to CRM197 presenting as a functionality a primary amine functionality of a lysine residue.

Thus, in a preferred embodiment of the present invention the carrier protein presents on its surface primary amino functionalities of lysine residues that are able to react with the functional group of the interconnecting molecule to provide modified carrier protein having on their surface said functional group of the interconnecting molecule, which is able to react with the Z group of the oligo- and polysaccharides of the invention.

Said functional group of the interconnecting molecules is for example selected from the group comprising or consisting of maleimide; α-iodoacetyl; α-bromoacetyl; and N-hydroxysuccinimide ester (NHS), aldehyde, imidoester, carboxylic acid, alkyl sulfonate, sulfonyl chloride, epoxide, anhydride, carbonate.

Other types of carrier include but are not limited to biotin or liposomes. The oligo- or polysaccharides conjugated to biotin or to a label are especially designed for diagnosing S. sonnei infections. As regards the use of a liposome as a carrier, in particular those, which do not imply covalent linkages, reference could be made to the International Application WO 2010/136947.

In a particular embodiment of the present invention, the carrier is biotin (as an anchor) or biotin/avidin complex.

In a particular embodiment of the present invention, the carrier is a multivalent scaffold, i.e. a carrier that enables multiple presentation of the oligo- or polysaccharide of the invention, in particular a scaffold able to form at least two bonds, each one with an oligo- or polysaccharide of the invention. Said multivalent scaffold is for example a linear polymer, a dendrimer, a monosaccharide, a cyclic peptide, or a (poly)-lysine scaffold, for example MAP (Multiple Antigen Peptide).

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

After conjugation, free and conjugated oligosaccharides can be separated. There are many suitable methods, including hydrophobic chromatography, tangential ultrafiltration, diafiltration, etc.

In a particular embodiment, the conjugate is chosen from:

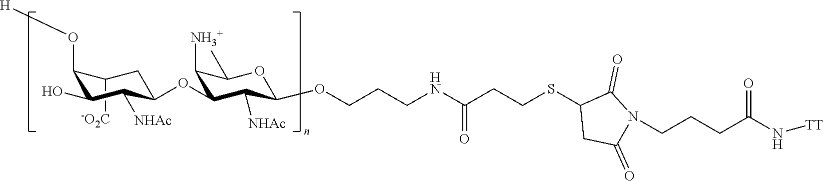

wherein:
n is 1, 2, 3 or 4;
TT is tetanus toxoid or a fragment thereof.

In another object, the invention provides an immunogenic composition comprising a conjugate according to the invention and a physiologically acceptable vehicle.

All the embodiments related to the conjugate apply here as well, alone or in combination.

The immunogenic (or vaccine) composition includes one or more pharmaceutically acceptable excipients or vehicles such as water, saline, glycerol, or ethanol. Additionally, auxiliary substances, such as wetting or emulsifying agents, pH buffering substances, and the like, may be present in such vehicles.

The glycoconjugates of the present invention which induce protective antibodies against S. sonnei infection are administered to a mammal subject, preferably a human, in an amount sufficient to prevent or attenuate the severity, extent of duration of the infection by S. sonnei.

Immunogenic compositions are suitable for administration to animal (and, in particular, human) patients, and thus include both human and veterinary uses. They may be used in a method of raising an immune response in a patient, comprising the step of administering the composition to the patient.

The immunogenic compositions of the present invention may be administered before a subject is exposed to S. sonnei and/or after a subject is exposed to S. sonnei.

Immunogenic compositions may be prepared in unit dose form. In some embodiments a unit dose may have a volume of between 0.1-1.0 mL e.g. about 0.5 mL.

The invention also provides a delivery device (e.g. syringe, nebulizer, sprayer, inhaler, dermal patch, etc.) containing an immunogenic composition of the invention e.g. containing a unit dose. This device can be used to administer the composition to a vertebrate subject.

The invention also provides a sterile container (e.g. a vial) containing an immunogenic composition of the invention e.g. containing a unit dose, or a multidoses sterile container.

The invention also provides a unit dose of an immunogenic composition of the invention.

The invention also provides a hermetically sealed container containing an immunogenic composition of the invention. Suitable containers include e.g. a vial.

Immunogenic compositions of the invention may be prepared in various forms. For example, the immunogenic compositions may be prepared as injectables, either as liquid solutions or suspensions. Solid forms suitable for solution in, or suspension in, liquid vehicles prior to injection can also be prepared (e.g. a lyophilized composition or a spray-freeze dried composition). The composition may be prepared for topical administration e.g. as an ointment, cream or powder. The composition may be prepared for oral administration e.g. as a tablet or capsule, as a spray, or as a syrup (optionally flavored). The composition may be prepared for pulmonary administration e.g. by an inhaler, using a fine powder or a spray. The composition may be prepared as a suppository. The composition may be prepared for nasal, aural or ocular administration e.g. as a spray or drops. Injectables for intramuscular administration are typical.

The pharmaceutical compositions may comprise an effective amount of an adjuvant i.e. an amount which, when administered to an individual, either in a single dose or as part of a series, is effective for enhancing the immune response to a co-administered S. sonnei type 2 antigen. This amount can vary depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the immunogenic composition, the treating doctor's assessment of the medical situation, and other relevant factors. The amount will fall in a relatively broad range that can be determined through routine trials.

Techniques for the formulation and administration of the immunogenic composition of the present invention may be found in “Remington's Pharmaceutical Sciences” Mack Publishing Co., Easton PA. Each vaccine dose comprises a therapeutically effective amount of oligo- or polysaccharide conjugate.

A therapeutically effective dosage of one conjugate according to the present invention or of one saccharide of general formula (I) refers to that amount of the compound that results in an at least a partial immunization against a disease. Toxicity and therapeutic efficacy of such compounds can be determined by standard pharmaceutical, pharmacological, and toxicological procedures in cell cultures or experimental animals. The dose ratio between toxic and therapeutic effect is the therapeutic index. The actual amount of the composition administered will be dependent on the subject being treated, on the subject's weight, the severity of the affliction, the manner of administration and the judgement of the prescribing physician.

Such amount will vary depending on the capacity of the subject to synthesize antibodies against the oligo- or polysaccharide, the degree of protection desired, the particular oligo- or polysaccharide conjugate selected and its mode of administration, among other factors. An appropriate effective amount can be readily determined by one skilled in the art. A therapeutically effective amount may vary in a wide range that can be determined through routine trials.

More particularly the oligo- or polysaccharide conjugate of the invention will be administered in a therapeutically effective amount that comprises from 0.1 μg to 100 μg, notably from 0.5 μg to 50 μg of oligo- or polysaccharide, preferably 1 μg to 10 μg. An optimal amount for a particular vaccine can be ascertained by methods known from the skilled in the art, in particular standard studies involving measuring the anti-S. sonnei antibody titers in subjects, more accurately protective antibody titers.

Methods of administering the immunogenic compositions of the invention are well known from the skilled in the art. Briefly, the immunogenic compositions of the invention may be administered in single or multiple doses. The inventors have found that the administration of a single dose of the immunogenic compositions of the invention may be sufficient. Alternatively, one unit dose followed by a second unit dose may be effective. Typically, the second (or third, fourth, fifth etc.) unit dose is identical to the first unit dose. The second unit dose may be administered at any suitable time after the first unit dose, in particular after 1, 2 or 3 months. In particular, following an initial administration, subjects may receive one or two booster injections at about four week intervals. For infants less than 12 months of age, two doses at not less than two month intervals can be administered, the first dose not being administered before 2 months of age. The immunogenic composition of the invention may include one or more adjuvants. However, the use of unadjuvanted compositions is also envisaged, for example, it may be advantageous to omit adjuvants in order to reduce potential toxicity. Accordingly, immunogenic compositions that do not contain any adjuvant or that do not contain any aluminium salt adjuvant are envisaged.

Adjuvants generally combined with glycoconjugate vaccines allow to strengthen the antibody response and hence the B response. Adjuvants can be added directly to the vaccine compositions or can be administered separately, either concurrently with or shortly after, administration of the vaccine.

Adjuvants are well known from the person skilled in the art. Reference is for instance made to Current Opinion in Immunology 2020, 65:97-101. Classically recognized examples of adjuvants include:
mineral-containing compositions, including calcium salts and aluminium salts (or mixtures thereof). Calcium salts include calcium phosphate. Aluminium salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt. The adjuvants known as aluminium hydroxide and aluminium phosphate may be also used. The invention can use any of the “hydroxide” or “phosphate” adjuvants that are in general used as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Mixtures of both an aluminium hydroxide and an aluminium phosphate can be employed in the formulation according to the present invention;
saponins, which are a heterologous group of sterol glycosides and triterpenoid glycosides that are found in the bark, leaves, stems, roots and even flowers of a wide range of plant species. Saponins from the bark of the Quillaia saponaria, Molina tree have been widely studied as adjuvants. Saponins can also be commercially obtained from Smilax ornata (sarsaprilla), Gypsophilla paniculata (brides veil), and Saponaria oficianalis (soap root). Saponin adjuvant formulations include purified formulations, such as QS21, as well as lipid formulations, such as ISCOMs. Saponin compositions have been purified using HPLC and RP-HPLC. Specific purified fractions using these techniques have been identified, including QS7, QS 17, QS 18, QS2 1, QH-A, QH-B and QH-C. Saponin formulations may also comprise a sterol, such as cholesterol. Combinations of saponins and cholesterols can be used to form unique particles called immunostimulating complexes (ISCOMs). ISCOMs generally include a phospholipid such as phosphatidylethanolamine or phosphatidylcholine. Any known saponin can be used in ISCOMs. Preferably, the ISCOM includes one or more of QuilA, QHA & QHC;
microparticles (i.e. a particle of 100 nm to 150 pm in diameter, more preferably 200 nm to 30 pm in diameter, or 500 nm to 10 pm in diameter) formed from materials that are biodegradable and non-toxic. Such non-toxic and biodegradable materials include, but are not restricted to poly(α-hydroxy acid), polyhydroxybutyric acid, polyorthoester, polyanhydride, polycaprolactone;
CD1d ligands, such as an α-glycosylceramide, phytosphingosine-containing α-glycosylceramides, OCH, KRN7000 [(2S,3S,4R)-1-O-(α-D-galactopyranosyl)-2-(N-hexacosanoylamino)-1,3,4-octadecanetriol], CRONY-101, 3″-sulfo-galactosyl-ceramide;
immunostimulatory oligonucleotides, such CpG motif containing ones (a dinucleotide sequence containing an unmethylated cytosine residue linked by a phosphate bond to a guanosine residue), or Cpl motif containing ones (a dinucleotide sequence containing cytosine linked to inosine), or a double-stranded RNA, or an oligonucleotide containing a palindromic sequence, or an oligonucleotide containing a poly(dG) sequence. Immunostimulatory oligonucleotides can include nucleotide modifications/analogs such as phosphorothioate modifications and can be double-stranded or (except for RNA) single-stranded;
compounds containing lipids linked to a phosphate-containing acyclic backbone, such as the TLR4 antagonist E5564;
bacterially derived ADP-ribosylating enterotoxins, in particular Mucosal Vaccine Adjuvant LT(R192G/L211A) or dmLT (as for example described in Clements et al., mSphere 2018, 3(4));
oil emulsions (e.g. Freund's adjuvant), in particular for diagnostic uses.

In particular, such adjuvants may be chosen from aluminium salts (aluminium hydroxide, aluminium phosphate), oil-in-water emulsion formulations with or without specific stimulating agents such as TLR agonists, muramyl peptides, saponin adjuvants, cytokines, detoxified mutants of bacterial toxins such as the cholera toxin, the pertussis toxin, or the E. coli heatlabile toxin.

The immunogenic composition of the invention may be administered with other immunogens or immunoregulatory agents, for example, immunoglobulins, cytokines, lymphokines and chemokines.

In a particular aspect, the immunogenic composition further comprises an immunogen which affords protection against another pathogen, such as for example, members of other Shigella species such as S. flexneri, for example S. flexneri serotype 1b, 2a, 3a, 6 (SF6) or 6a (SF6a), and S. dysenteriae type 1, or pathogens responsible for diarrhoeal disease in humans.

In another particular aspect, the immunogenic composition is devoid of an immunogen which affords protection against another pathogen, such as for example, members of other Shigella species such as S. flexneri, for example S. flexneri serotype 1b, 2a, 3a, 6 (SF6) or 6a (SF6a), and/or S. dysenteriae type 1, and/or pathogens responsible for diarrhoeal disease in humans.

Immunogenic compositions are preferably in aqueous form, particularly at the point of administration, but they can also be presented in non-aqueous liquid forms or in dried forms e.g. as gelatin capsules, or as lyophilisates, etc.

Immunogenic compositions may include one or more preservatives, such as thiomersal or 2-phenoxyethanol. Mercury-free compositions are preferred, and preservative-free vaccines can be prepared.

Immunogenic compositions may include a physiological salt, such as a sodium salt e.g. to control tonicity. Sodium chloride (NaCl) is typical and may be present at between 1 and 20 mg/ml. Other salts that may be present include potassium chloride, potassium dihydrogen phosphate, disodium phosphate dehydrate, magnesium chloride, calcium chloride, etc.

Immunogenic compositions can have an osmolality of between 200 mOsm/kg and 400 mOsm/kg.

Immunogenic compositions may include compounds (with or without an insoluble metal salt) in plain water (e.g. w.f.i.), but will usually include one or more buffers. Typical buffers include: a phosphate buffer; a Tris buffer; a borate buffer; a succinate buffer; a histidine buffer (particularly with an aluminium hydroxide adjuvant); or a citrate buffer. Buffer salts will typically be included in the 5-20 mM range.

Immunogenic compositions typically have a pH between 5.0 and 9.5 e.g. between 6.0 and 8.0.

Immunogenic compositions are preferably sterile and gluten free.

Typically, the immunogenic compositions are prepared as injectables either as liquid solutions or suspensions; or as solid forms suitable for solution or suspension in a liquid vehicle prior to injection. The preparation may be emulsified or encapsulated in liposomes for enhanced adjuvant effect. In this respect, reference could be made to International Application WO 2010/136947.

Once formulated, the immunogenic compositions may be administered parenterally, by injection, either subcutaneous, intramuscular or intradermal.

Typically, the immunogenic compositions of the invention may be administered intramuscularly, e.g. by intramuscular administration to the high or the upper arm. Alternative formulations suitable for other mode of administration include oral and intranasal formulations.

In another aspect, the invention concerns a conjugate or an immunogenic composition as defined above for use in vaccination.

In another aspect, the invention concerns a conjugate or an immunogenic composition as defined above for use in vaccination.

In another aspect, the invention concerns a conjugate or an immunogenic composition as defined above for use in vaccination against S. sonnei infection and/or infection caused by pathogens featuring cross-reactive carbohydrate antigens, for example a Plesiomonas shigelloides infection, notably a P. shigelloides O17 infection.

In another aspect, the invention concerns a compound of the following formula:

Q-(B)ₓ-(AB)ₙ-(A)_{y}-OR (IIa) or

Q-(A)ₓ-(BA)ₙ-(B)_{y}—OR (IIb),

wherein:
x is 0 or 1,
y is 0 or 1,
n ranges from 1 to 50,
Q is H or a C₁-C₆ alkyl,
A is 4)-α-L-AltpNAcA-(1→,
B is 3)-β-D-FucpNAc4N-(1→,
R is H, C₁-C₆ alkyl, in particular propyl or methyl, or a group LZ,
L is:
   a single bond,
   a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, or
   a divalent C₁-C₁₂ alkyl, C₂-C₁₂ alkenyl or C₂-C₁₂ alkynyl chain substituted by at least one —OH group, being in particular of the following formula —(CH₂—CH₂—C(OH))_{q}—(CH₂—CH₂)ᵢ, wherein i is 0 or 1 and q ranges from 1 to 10,
   N(R^{a})-D-, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; D is C₁-C₇-alkylene, C₁-C₇-alkoxy, C₁-C₄-alkyl-(OCH₂CH₂)ₚO—C₁-C₄-alkyl, O—C₁-C₄-alkyl-(OCH₂CH₂)ₚO—C₁-C₄-alkyl or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1;
Z is Z₁ or F₁-L₂-Z₂,
Z₁ is a terminal (reactive) function or group, optionally protected, able to form a covalent bond with a carrier and/or a solid support, or a multivalent scaffold; an anchor; a mono-, oligo- or polysaccharide; or a dye or fluorescent residue.
F₁ is any group enabling to bond the linker L to the linker L₂, F₁ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following:

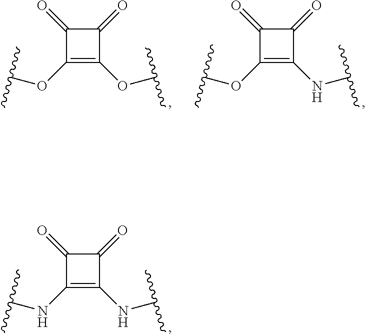 [SMILES: *Oc1c(O*)c(=O)c1=O ; *Nc1c(O*)c(=O)c1=O ; *Nc1c(N*)c(=O)c1=O]

L₂ is a single bond, divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
Z₂ is Z₁ or F₂-L₃-Z₁,
F₂ is any group enabling to bond the linker L to the linker L₃, F₂ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following:

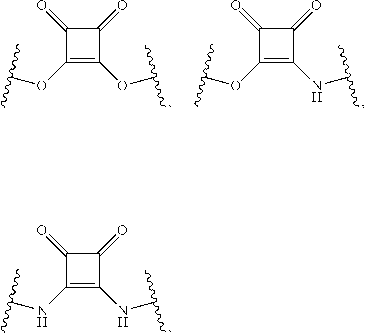 [SMILES: *Oc1c(O*)c(=O)c1=O ; *Nc1c(O*)c(=O)c1=O ; *Nc1c(N*)c(=O)c1=O]

L₃ is single bond, a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
or a pharmaceutically acceptable salt thereof,
with the proviso that said compound is not H-AB—OPr, H—BA-OPr, H-ABA-OPr, H—BAB—OPr, H-(AB)₂—OPr or H-BA-OMe.

All the embodiments related to the conjugate or the immunogic composition apply here as well, alone or in combination.

The LZ group may be of one of the following formulae:
L-Z₁;
L-F₁-L₂-Z₁; or
L-F₁-L₂-F₂-L₃-Z₁.

In a particular embodiment, Z₁ is a terminal (reactive) function or group, optionally protected, able to form a covalent bond with a carrier and/or a solid support.

In particular embodiment, the carrier and/or a solid support presents on its surface functionalities, more particularly primary amino functionalities, notably of lysine residues, that are able to react with Z₁.

In another particular embodiment, the carrier and/or a solid support presents on its surface functionalities, more particularly primary amino functionalities, notably of lysine residues, linked to an interconnecting molecule, which is able to react with the Z₁ group of the oligo- and polysaccharides of the invention.

In a particular embodiment, Z₁ is a multivalent scaffold; an anchor; a mono-, oligo- or polysaccharide; or a dye or fluorescent residue.

In a particular embodiment, L₂ is a single bond, and F₁ and Z₂ or Z₁ are one and only group.

In a particular embodiment, L₃ is a single bond, and F₂ and Z₁ are one and only group.

By anchor is in particular meant a residue able to form a non-covalent type attachment with a carrier and/or a solid support. Said anchor is for example biotine, able to form non-covalent bonds with streptavidine bound to a solid support.

By multivalent scaffold is in particular meant a scaffold able to form at least two bonds, each one with one compound of formula (II) of the present invention. Said multivalent scaffold is for example a linear polymer, a dendrimer, a monosaccharide, a cyclic peptide, or a (poly)-lysine scaffold.

In a particular embodiment, Z₁ is a terminal (reactive) function or group, optionally protected, able to form a covalent bond with a carrier and/or a solid support.

In a particular embodiment, the invention concerns a compound of the following formula:

Q-(B)ₓ-(AB)ₙ-(A)_{y}-OR (IIa)

with the proviso that:
when n=1, then LZ is not Pr;
when n=1, x=0 and y=0, then LZ is not Me;
when n=2, x=0 and y=0, then LZ is not Pr.

In a particular embodiment, n ranges from 1 to 50, more particularly:
from 1 or 2 to 25; or
from 1 or 2 to 12; or
from 1 or 2 to 10; or
from 1 or 2 to 4; or
from 2 or 3 to 8.

In a more particular embodiment, n is 1, 2, 3, 4, 5, 6, 7, 8, 9 or 10,

In a particular embodiment, Q is H.

In another particular embodiment, Q is Me.

In a particular embodiment, R is H. In this case, the compound of the invention is a hemiacetal.

In a particular embodiment, R is Pr.

In a particular embodiment, R is LZ.

In a particular embodiment, R is not Pr when n is 1 or 2.

In a particular embodiment, Q is H and R is H.

In a particular embodiment, Q is H and R is Pr.

In a particular embodiment, Q is H and R is LZ. In a particular embodiment, LZ is:
L-Z₁;
L-F₁-L₂-Z₁; or
L-F₁-L₂-F₂-L₃-Z₁.

In a particular embodiment, Z₁ is a halogen, in particular Cl, Br, I, more particularly Br, biotin, C₂-C₆ alkenyl, C₂-C₆ alkynyl, azido, alkoxy, epoxide, C(═O)H, hemiacetal, C(═O)R_{c}, acetal, SRₐ, NH₂ or NHC(═O)CH₂Hal, wherein Hal is a halogen, in particular Cl, Br, I, more particularly Br,
Rₐ being H, C(═O)CH₃ or SR_{b}, and
R_{b} being a C₁-C₆ alkyl, a C₆-C₁₀ aryl, optionally substituted, in particular by one or more C₁-C₆alkyl groups, a 5 to 7 membered heteroaryl, such as pyridyl, optionally substituted, in particular by one or more C₁-C₆ alkyl groups, any group allowing to convert SSR_{b} into SH, or Q-(B)ₓ-(A-B)ₙ-(A)_{y}-O—,
R_{c} being a C₁-C₆ alkyl.

In a particular embodiment, LZ is LZ₁, with L being a divalent C₁-C₁₂ alkyl and Z being C(═O)H, or a protected C(═O)H such as a hemiacetal or C(OH)—CH₂—OH group.

In a more particular embodiment, LZ is CH₂—C(═O)H or CH₂—C(OH)—CH₂—OH group.

In a particular embodiment, LZ is LZ₁, with L being a divalent C₁-C₁₂ alkyl and Z being C(═O)R^{a}, or a protected C(═O)R^{a} such as an acetal.

In a more particular embodiment, LZ is CH₂—CH₂—C(═O)—CH₃ or the corresponding group wherein the ketone is protected as an acetal.

In a particular embodiment, LZ is LZ₁, with L being a divalent C₁-C₁₂ alkyl, in particular a C₃ alkyl, and Z being NH₂, or NH₃⁺.

In a particular embodiment, L is a divalent C₁-C₁₂ alkyl, in particular —(CH₂)₃—, and Z is F₁-L₂-Z₂, with F₁ being an amide, L₂ being divalent C₁-C₁₂ alkyl, in particular —CH₂—, and Z₂ being —SH or a protected thiol such as —SAc.

In a particular embodiment, L is a divalent C₁-C₁₂ alkyl, in particular —(CH₂)₃—, and Z is F₁-L₂-Z₂, with F₁ being an amide, L₂ being divalent C₁-C₁₂ alkyl, in particular —(CH₂)₂—, and Z₂ being —SH or a protected thiol such as —S—S-pyridine.

In a particular embodiment, L is a divalent C₁-C₁₂ alkyl, in particular —CH₂—, and Z is F₁-L₂-Z₂, with F₁ being a hydrazonamide, in particular —C═N—NH—C(═O)—, L₂ being divalent C₁-C₁₂ alkyl, in particular —(CH₂)₂—, and Z₂ being —SH or a protected thiol such as —S—S-pyridine.

In a particular embodiment, L is a divalent C₁-C₁₂ alkyl, in particular —CH₂—, and Z is F₁-L₂-Z₂, with F₁ being a hydrazone, in particular —C═N—NH—, L₂ being a single bond, and Z₂ being as follows:

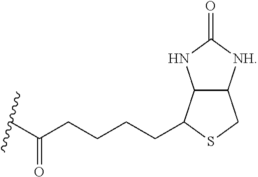 [SMILES: *C(=O)CCCCC1SCC2NC(=O)NC21]

In a particular embodiment, L is —N(R^{a})-D-E-CH₂—(CH₂)_{q}—S— or LZ is —N(R^{a})-D-E-CH₂—(CH₂)_{q}-SH, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; D is C₁-C₇-alkylene, C₁-C₇-alkoxy, C₁-C₄-alkyl-(OCH₂CH₂)ₚO—C₁-C₄-alkyl, O—C₁-C₄-alkyl-(OCH₂CH₂)ₚO—C₁-C₄-alkyl or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1; E is NHC(O), S or CH₂; q is 0 to 6, preferably q is 1, 2, 3 or 4, and further preferably q is 1 or 2.

In a particular embodiment, the invention provides oligo- or polysaccharide selected from the group consisting of:

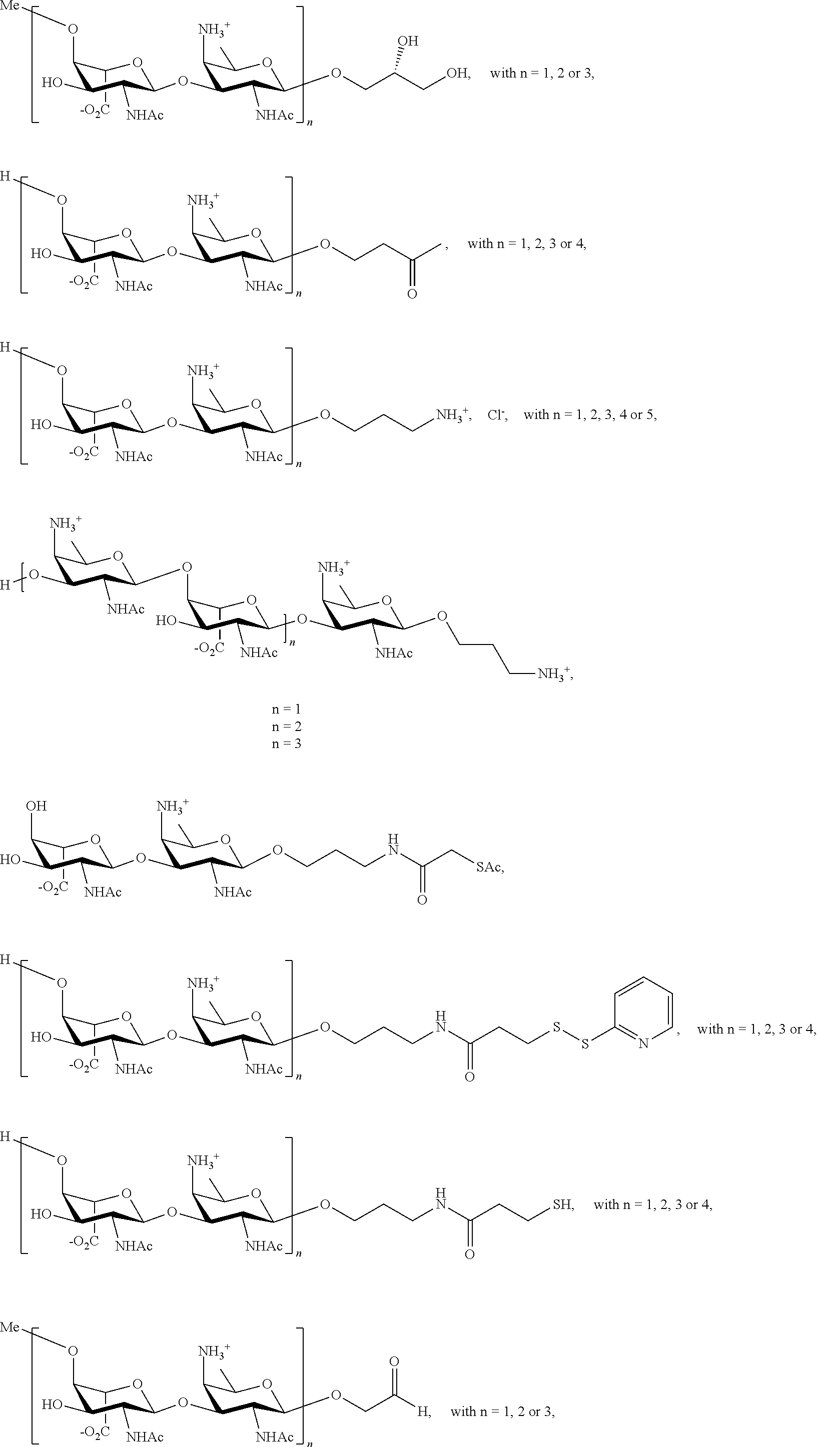

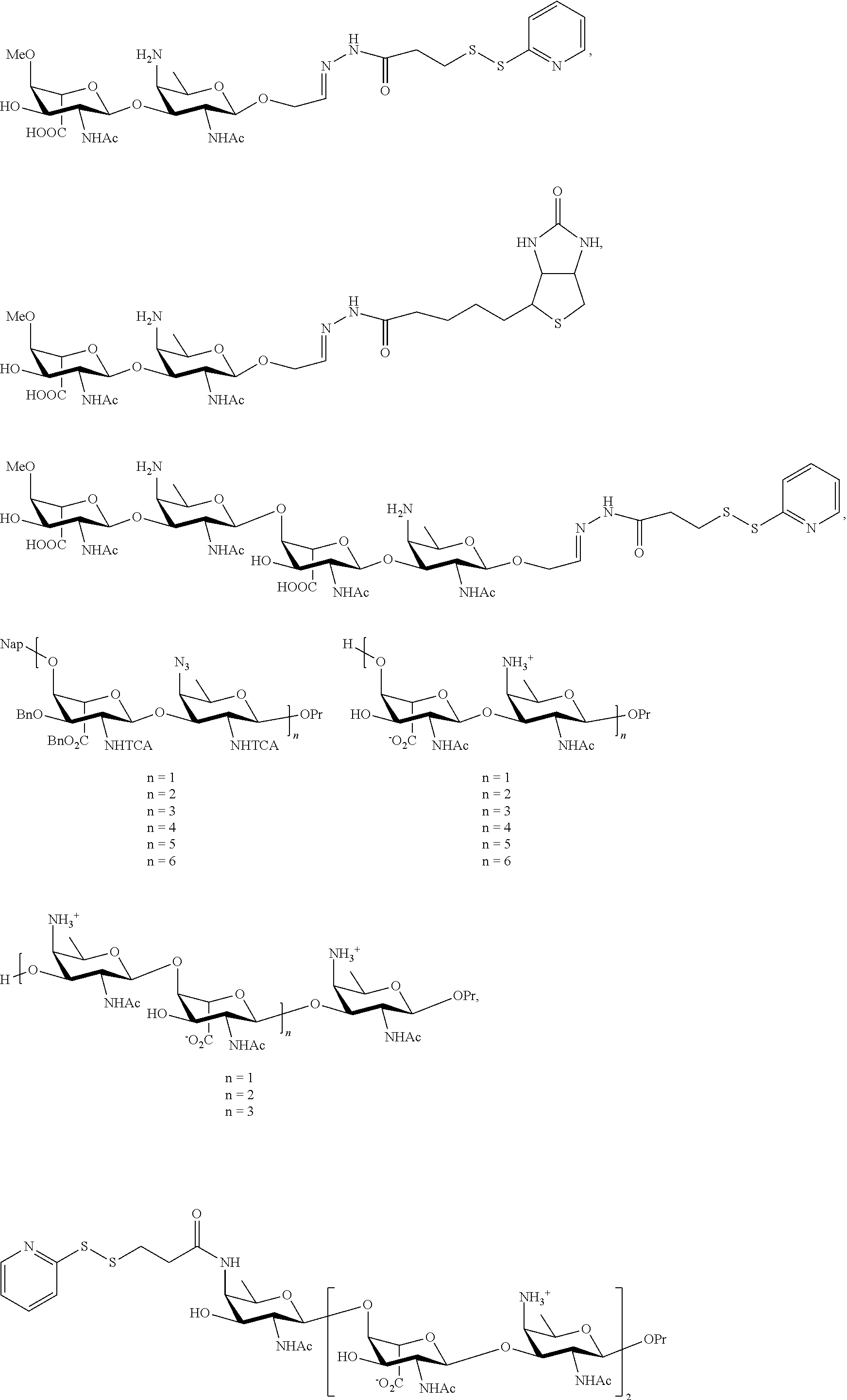

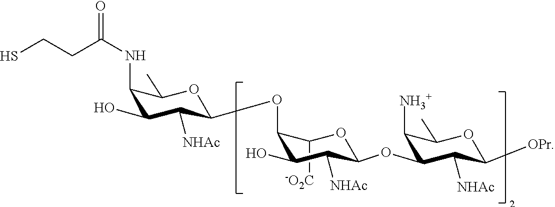

In another aspect, the present invention relates to a kit for the in vitro diagnostic of S. sonnei infection, wherein said kit comprises an oligo- or polysaccharide as defined herein, in particular compounds of formula (IIa) or (IIb), optionally bound to a label or a solid support.

In a particular embodiment, the oligo- or polysaccharides according to the present invention, in particular compounds of formula (IIa) or (IIb), are used, in vitro, as S. sonnei specific diagnostic reagents in standard immunoassays.

Alternatively, the oligo- or polysaccharides according to the present invention, in particular compounds of formula (IIa) or (IIb), are used to test the presence of S. sonnei-specific antibodies. Oligo- or polysaccharides, in particular compounds of formula (IIa) or (JIb), may be used for epidemiological studies, for example for determining the geographic distribution and/or the evolution of S. sonnei infection worldwide, as well as for evaluating the S. sonnei-specific antibody response induced by an immunogen.

The oligo- or polysaccharides according to the present invention, in particular compounds of formula (IIa) or (IIb), may be advantageously labelled and/or immobilized onto a solid phase, according to standard protocols known to the man skilled in the art. Such labels include, but are not limited to, enzymes (alkaline phosphatase, peroxydase), luminescent or fluorescent molecules. For example an oligo- or polysaccharide conjugated to biotine, according to the present invention may be immobilized onto a solid phase, to detect the presence of S. sonnei-specific antibodies in biological samples.

Such immunoassays include, but are not limited to, agglutination assays, radioimmunoassay, enzyme-linked immunosorbent assays, fluorescence assays, western-blots and the like.

Such assays may be for example, of direct format (where the labelled oligo- or polysaccharide is reactive with the antibody to be detected), an indirect format (where a labelled secondary antibody is reactive with said oligo- or polysaccharide), or a competitive format (addition of a labelled oligo- or polysaccharide).

For all therapeutic, prophylactic and diagnostic uses, the oligo- or polysaccharides of the invention, in particular compounds of formula (IIa) or (IIb), alone or linked to a carrier, as well as antibodies and other necessary reagents and appropriate devices and accessories may be provided in kit form so as to be readily available and easily used.

In another aspect, the present invention relates to the use of an oligo- or polysaccharide as defined herein, in particular a compound of formula (IIa) or (IIb), for in vitro diagnostic.

In another aspect, the present invention relates to the use of a compound of the following formula (I₀):

T-A′-B′—Y or T-B′-A′-Y (I₀),

### Wherein:

T is chosen from 2-naphtylmethyl (Nap), para-methoxybenzyl (PMB), 4-bromobenzyl (PBB), benzyloxymethyl acetal (BOM), 2-methoxyethoxymethylether (MEM), methoxypropyl (MOP), tetrahydropyranyl (THP), allyl (All), C₁-C₆ alkyl, or a silyl, T being in particular tert-butyldimethylsilyl (TBS), dimethylhexylsilyl (TDS), triisopropyl silyl (TIPS), or triethylsilyl (TES),
Y is chosen from:
   OAll, when T is not All;
   Silyl ethers, in particular tert-butyldimethylsilyl ether (OTBS), dimethylhexylsilyl ether (OTDS), triethylsilyl ether (OTES), triisopropyl silyl ether (OTIPS), when T is Nap or PMB;
   OPMB, ONap, when OT is a silyl ether or T is All or PBB ether;
   p-methoxyphenyl-O (OMP or OPMP); and
   SR₄, wherein R₄ is such as the compound is a thioglycoside;
A′ is

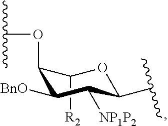

in particular

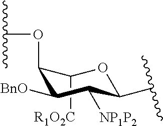

wherein:
   P₁ is chosen from TCA, TFA, DCA, ClCH₂—C(═O)—(C_{Ar}), Ac, benzyloxycarbamate (Cbz), Trichloroethoxycarbonyl (Troc), and Fmoc, at least one P₁ and P₂ being chosen from TCA, DCA, Ac, Fmoc, Troc, and, when Y is not OAll, OAlloc,
   P₂ is H or chosen from Ac, Boc, TFA, benzyloxycarbamate (Cbz), and 2,2,2-trichloroethoxycarbonyl (Troc), P₂ being H when P₁ is not Ac,
   or P₁ and P₂ form together a phthalimido or a tetrachlorophthalimido (Cl₄Phth) group,
   R₂ is CO₂R₁ or CH₂OR₃, wherein R₃ is Ac, benzoyl (Bz), or R₃ forms with T a benzylidene group,
   R₁ is chosen from C₁-C₆ alkyl, notably Me or tert-butyl (tBu), Bn and p-methoxybenzyl (PMB) groups, R₁ being in particular Bn,
B′ is

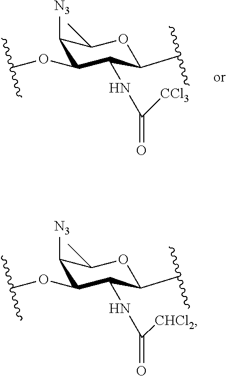

in particular

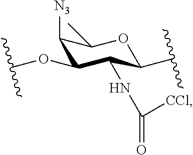

for the preparation of a compound of the following formula (II):

Q-(B)ₓ-(AB)ₙ-(A)_{y}-OR (IIa) or

Q-(A)ₓ-(BA)ₙ-(B)_{y}—OR (IIb),

wherein:
x is 0 or 1,
y is 0 or 1,
n ranges from 1 to 50,
Q is H or a C₁-C₆ alkyl,
A is 4)-α-L-AltpNAcA-(1→,
B is 3)-β-D-FucpNAc4N-(1→,
R is H, C₁-C₆ alkyl, in particular propyl or methyl, or a group LZ,
L is:
   a single bond,
   a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, or
   a divalent C₁-C₁₂ alkyl, C₂-C₁₂ alkenyl or C₂-C₁₂ alkynyl chain substituted by at least one —OH group, being in particular of the following formula —(CH₂—CH₂—C(OH))_{q}—(CH₂—CH₂)ᵢ, wherein i is 0 or 1 and q ranges from 1 to 10,
   —N(R^{a})-D-, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; D is C₁-C₇-alkylene, C₁-C₇-alkoxy, C₁-C₄-alkyl-(OCH₂CH₂)ₚO—C₁-C₄-alkyl, O—C₁-C₄-alkyl-(OCH₂CH₂)ₚO—C₁-C₄-alkyl or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1;
Z is Z₁ or F₁-L₂-Z₂,
Z₁ is a terminal (reactive) function or group, optionally protected, able to form a covalent bond with a carrier and/or a solid support,
F₁ is any group enabling to bond the linker L to the linker L₂, F₁ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following:

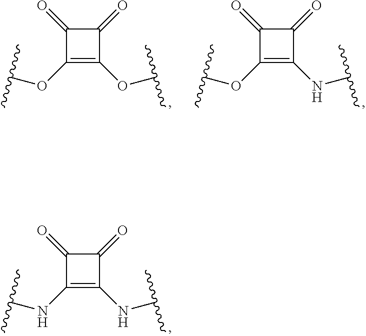 [SMILES: *Oc1c(O*)c(=O)c1=O ; *Nc1c(O*)c(=O)c1=O ; *Nc1c(N*)c(=O)c1=O]

L₂ is a single bond, divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
Z₂ is Z₁ or F₂-L₃-Z₁,
F₂ is any group enabling to bond the linker L to the linker L₃, F₂ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following:

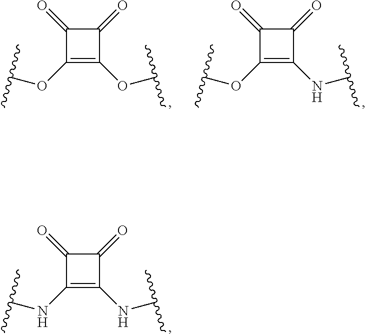 [SMILES: *Oc1c(O*)c(=O)c1=O ; *Nc1c(O*)c(=O)c1=O ; *Nc1c(N*)c(=O)c1=O]

L₃ is single bond, a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
in particular with the proviso that said compound is not H-AB—OPr, H—BA-OPr, H-ABA-OPr, H—BAB—OPr, H-(AB)₂—OPr or H-BA-OMe.
TCA is Cl₃C—C(═O)—.
TFA is F₃C—C(═O)—.
DCA is Cl₂CH—C(═O)—.
CA is ClCH₂—C(═O)—.

In the whole specification, and in particular about A′, the Bn protecting group may be replaced by a Nap protecting group.

All the embodiments related to the conjugate, the immunogenic composition or the compounds of formula (IIa) or (IIb) apply here as well, alone or in combination.

The mono-, oligo- or polysaccharide is for example a mono-, oligo- or polyglucosamine, or a beta-glucan.

Said compound T-A′-B′—Y or T-B′-A′-Y (I₀), can be used to prepare the acceptor H-A′-B′—Y or H—B′-A′-Y, or the donor T-A′-B′—X or T-B′-A′-X, or the hemiacetal intermediate T-A′-B′—OH or T-B′-A′-OH as defined below.

In another aspect, the present invention relates to the use of a compound of the following formula T-A′-B′—OH or T-B′-A′-OH for the preparation of a compound of the following formula (II) Q-(B)ₓ-(AB)ₙ-(A)_{y}-OR (IIa) or Q-(A)ₓ-(BA)ₙ-(B)_{y}—OR (IIb).

In another aspect, the present invention relates to the use of a compound of the following formula T-A′-B′—X or T-B′-A′-X for the preparation of a compound of the following formula (II) Q-(B)ₓ-(AB)ₙ-(A)_{y}-OR (IIa) or Q-(A)ₓ-(BA)ₙ-(B)_{y}—OR (IIb). In the whole description, when a compound comprises the following sequence:

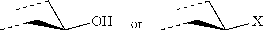

with X being an imidate as defined in the present specification,

Said compound may in fact correspond to a compound with, respectively:

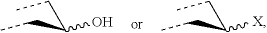

wherein the wavy bond indicates that the corresponding substituent is in axial and/or in equatorial position.

Thus, a compound containing such a wavy bond exist as a mixture of the alpha and beta anomers, or only as the alpha or beta anomer.

Unless specified otherwise, A′ and/or B′, in particular A′ can be in another conformation than the one indicated in the formulae. More particularly, the pyranose ring of A′ can be in another conformation than the one indicated in the formula, and for example chosen from the chair, boat and skewed conformations.

In a particular embodiment,

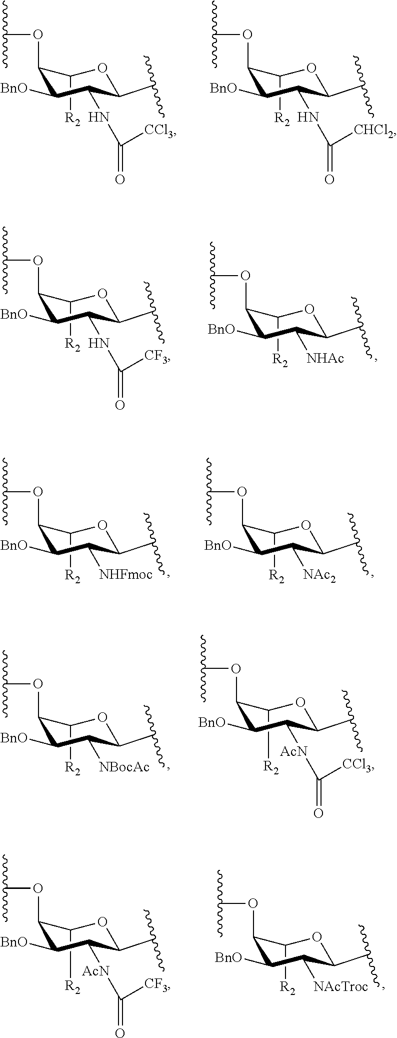

more particularly

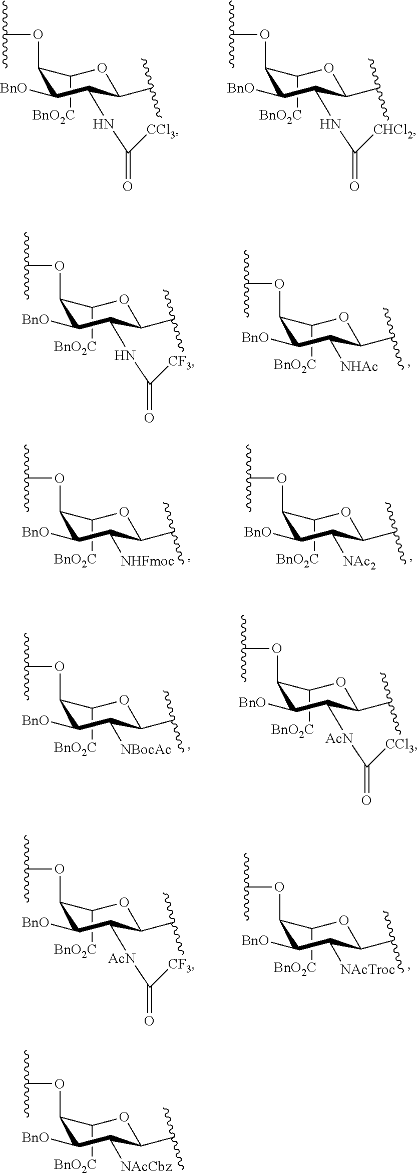

In a particular embodiment, T is chosen from 2-naphtylmethyl (Nap), para-methoxybenzyl (PMB), 4-bromobenzyl (PBB), benzyloxymethyl acetal (BOM), allyl (All), C₁-C₆alkyl, or a silyl, T being in particular tert-butyldimethylsilyl (TBS), dimethylhexylsilyl (TDS), triisopropyl silyl (TIPS), or triethylsilyl (TES),

In a particular embodiment, T is 2-naphtylmethyl (Nap), para-methoxybenzyl (PMB) or C₁-C₆ alkyl, in particular 2-naphtylmethyl (Nap), methoxymethylether (MEM), methyl ether (Me), tetrahydropyranyl acetal (THP).

In a particular embodiment, T is chosen from 2-naphtylmethyl (Nap), para-methoxybenzyl (PMB), 4-bromobenzyl (PBB), benzyloxymethyl acetal (BOM), or is such as OT is a silyl ether, T being in particular tert-butyldimethylsilyl (TBS), dimethylhexylsilyl (TDS), triisopropylsilyl (TIPS), or triethylsilyl (TES).

In a particular embodiment, T is chosen from 2-naphtylmethyl (Nap), 4-bromobenzyl (PBB), benzyloxymethyl acetal (BOM), C₁-C₆ alkyl, or is such as OT is a silyl ether, T being in particular tert-butyldimethylsilyl (TBS), dimethylhexylsilyl (TDS), triisopropylsilyl (TIPS), or triethylsilyl (TES).

In a particular embodiment, T is chosen from 2-naphtylmethyl (Nap), para-methoxybenzyl (PMB), C₁-C₆ alkyl, or tert-butyldimethylsilyl (TBS), in particular 2-naphtylmethyl (Nap).

In a particular embodiment, A′ is

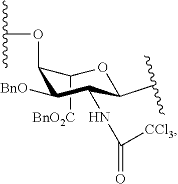

### and B′ is

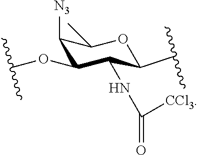

In a particular embodiment, A′ is

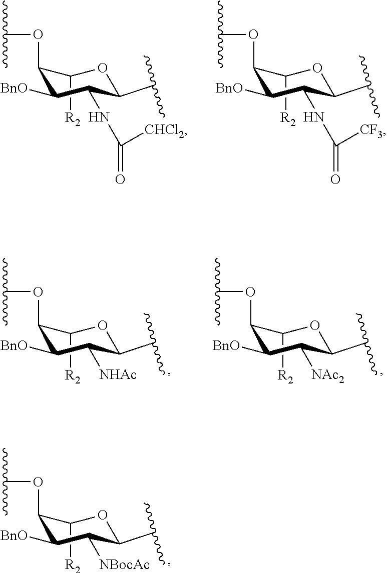

more particularly

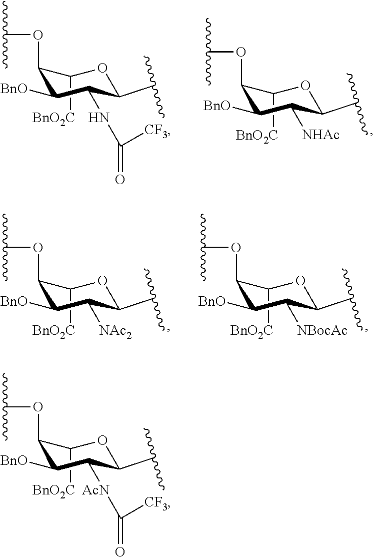

### and B′ is

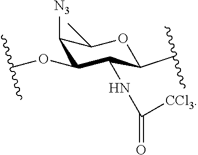

In a particular embodiment, Y is OAll.

In a particular embodiment, Y is OAll, and T is Nap, PMB, PBB, BOM, C₁-C₆ alkyl, or is such as OT is a silyl ether, T being in particular Nap, PMB, PBB, BOM, or is such as OT is a silyl ether, T being more particularly Nap or PMB, preferably Nap.

In a particular embodiment, Y is OAll, T is Nap, PMB, PBB, BOM, C₁-C₆ alkyl, or is such as OT is a silyl ether, T being in particular Nap, PMB, PBB, BOM, or is such as OT is a silyl ether, T being more particularly Nap or PMB, preferably Nap, A′ is

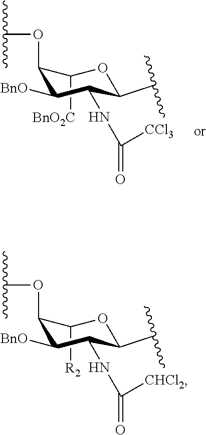

in particular

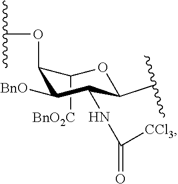

### and B′ is

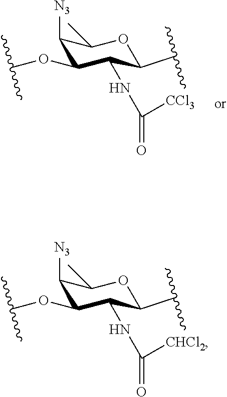

in particular

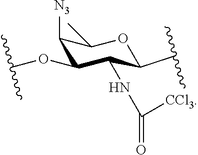

In a particular embodiment, Y is a silyl ethers, in particular tert-butyldimethylsilyl (TBS), dimethylhexylsilyl (TDS), triethylsilyl (TES), triisopropylsilyl (TIPS) and T is Nap or PMB.

In a particular embodiment, Y is OPMB, and OT is a silyl ether, in particular tert-butyldimethylsilyl (TBS), dimethylhexylsilyl (TDS), triethylsilyl (TES), triisopropylsilyl (TIPS).

In a particular embodiment, Y is SR₄.

Thioglycosides are well known from the skilled in the art. Reference is made for example to Advances in Carbohydrate Chemistry and Biochemistry, Volume 52, 1997, Pages 179-205.

In a particular embodiment, R₄ is chosen from:
C₁-C₁₂-alkyl, in particular Me or Et;
C₁-C₁₂-alkyl-Ar, wherein Ar is an aryl, optionally substituted, notably by one or more groups chosen from C₁-C₆ alkyl, O—C₁-C₆ alkyl, NO₂, in particular (CH₂)₃-Ph, CH₂-(tert-butyl-Ph) (MBP),
C₁-C₁₂-alkyl-Het, wherein Het is a heteroaryl, optionally substituted, notably by one or more groups chosen from C₁-C₆ alkyl, O—C₁-C₆ alkyl, NO₂,
C₁-C₁₂-alkenyl, in particular Me or Et;
C₁-C₁₂-alkenyl-Ar, wherein Ar is an aryl, optionally substituted, notably by one or more groups chosen from C₁-C₆ alkyl, O—C₁-C₆ alkyl, NO₂, in particular 4-(p-Methoxyphenyl)-4-pentenyl (MPTG),
C₁-C₁₂-alkenyl-Het, wherein Het is a heteroaryl, optionally substituted, notably by one or more groups chosen from C₁-C₆ alkyl, O—C₁-C₆ alkyl, NO₂,
aryl, optionally substituted, notably by one or more groups chosen from C₁-C₆ alkyl, O—C₁-C₆ alkyl, NO₂, in particular phenyl, tolyl, -Ph-NO₂;
heteroaryl, optionally substituted, notably by one or more groups chosen from C₁-C₆alkyl, O—C₁-C₆ alkyl, NO₂, in particular pyridyl, indolyl, benzoxazolyl (Box); and
glycosyl;
or is such as SR₄ is an alkoxythioimidate.

In a particular embodiment, R₂ is CO₂R₁, with R₁ being in particular Bn.

In a particular embodiment, Z is a halogen, in particular Cl, Br, I, more particularly Br, biotin, C₂-C₆ alkenyl, C₂-C₆ alkynyl, azido, alkoxy, epoxide, acetal, C(═O)H, SRₐ, NH₂ or NHC(═O)CH₂Hal, wherein Hal is a halogen, in particular Cl, Br, I, more particularly Br,
Rₐ being H, C(═O)CH₃ or SR_{b}, and
R_{b} being a C₁-C₆ alkyl, a C₆-C₁₀ aryl, optionally substituted, in particular by one or more C₁-C₆alkyl groups, a 5 to 7 membered heteroaryl, such as pyridyl, optionally substituted, in particular by one or more C₁-C₆ alkyl groups, any group allowing to convert SSR_{b} into SH, or Q-(B)ₓ-(A-B)ₙ-(A)_{y}-O—. In this last case, the compound of the invention is a dimer version of the sugar of the invention.

Thus, Z can establish non-covalent bonds (biotin) or covalent bonds through chemical reactions well known from the skilled in the art.

Some of these reactions are nucleophilic substitutions well known from the skilled in the art, and involve for example halogen, alkoxy, epoxide, SRₐ, NH₂ or NHC(═O)CH₂Hal, Hal being more particularly Br, groups.

Some other of these reaction are known as “click reaction” and involve for example C₂-C₆alkenyl, C₂-C₆ alkynyl, azido, C(═O)H, or SRₐ groups, or hydrazide/hydrazone, oxime-mediated reactions. Examples of these reactions can for instance be found in Chem. Soc. Rev. (2014) 43, 7013-7039.

As an illustration, Z can be an azido, which can for example form a covalent through a strain promoted alkyne-azide cycloaddition (SPAAC), also termed as Cu-free or non coper-based click reaction, or through copper(I)-catalyzed alkyne-azide cycloaddition (Glycoconj J. (2011) 28(3-4):149-164).

As another illustration, Z can be a thiol group that can be involved in thiol-selective bioconjugation reactions, in particular a thiol-maleimide or a thiol-bromoacetamide reaction.

Examples of these reactions can for instance be found in Curr. Opin. Chem. Biol. (2020) 58, 28-36.

As another illustration, Z can be a maleimide that can be involved in thiol-selective bioconjugation reactions, in particular a thiol-maleimide reaction. Examples of these reactions can for instance be found in Science 2004 Jul. 23; 305(5683):522-5

Reference is also made to Chem. Soc. Rev., 2018, 47, 9015; and to Chem. Soc. Rev., 2016, 45, 1691; regarding conjugation of oligo- and polyglucosides to a carrier.

In another aspect, the invention concerns a process of preparation of a compound of the following formula (II):

Q-(B)ₓ-(AB)ₙ-(A)_{y}-OR (IIa) or

Q-(A)ₓ-(BA)ₙ-(B)_{y}—OR (IIb),

wherein:
x is 0 or 1,
y is 0 or 1,
n ranges from 1 to 50,
Q is H or a C₁-C₆ alkyl,
A is 4)-α-L-AltpNAcA-(1→,
B is 3)-β-D-FucpNAc4N-(1→,
R is H, C₁-C₆ alkyl, in particular propyl or methyl, or a group LZ,
L is:
   a single bond,
   a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, or
   a divalent C₁-C₁₂ alkyl, C₂-C₁₂ alkenyl or C₂-C₁₂ alkynyl chain substituted by at least one —OH group, being in particular of the following formula —(CH₂—CH₂—C(OH))_{q}—(CH₂—CH₂)ᵢ, wherein i is 0 or 1 and q ranges from 1 to 10,
   —N(R^{a})-D-, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; D is C₁-C₇-alkylene, C₁-C₇-alkoxy, C₁-C₄-alkyl-(OCH₂CH₂)ₚO—C₁-C₄-alkyl, O—C₁-C₄-alkyl-(OCH₂CH₂)ₚO—C₁-C₄-alkyl or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1;
Z is Z₁ or F₁-L₂-Z₂,
Z₁ is a terminal (reactive) function or group, optionally protected, able to form a covalent bond with a carrier and/or a solid support, or a multivalent scaffold; an anchor; a mono-, oligo- or polysaccharide; or a dye or fluorescent residue.
F₁ is any group enabling to bond the linker L to the linker L₂, F₁ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following:

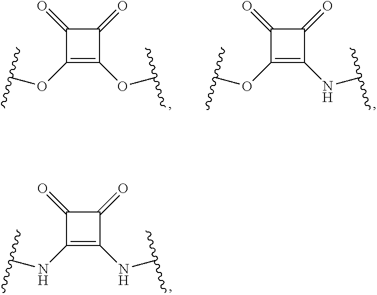 [SMILES: *Oc1c(O*)c(=O)c1=O ; *Nc1c(O*)c(=O)c1=O ; *Nc1c(N*)c(=O)c1=O]

L₂ is a single bond, divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
Z₂ is Z₁ or F₂-L₃-Z₁,
F₂ is any group enabling to bond the linker L to the linker L₃, F₂ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following:

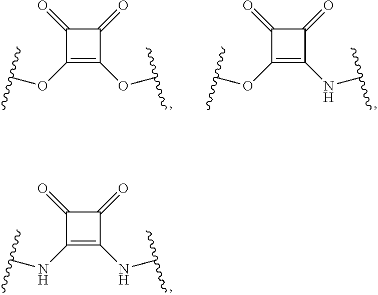 [SMILES: *Oc1c(O*)c(=O)c1=O ; *Nc1c(O*)c(=O)c1=O ; *Nc1c(N*)c(=O)c1=O]

L₃ is single bond, a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, in particular with the proviso that said compound is not H-AB—OPr, H—BA-OPr, H-ABA-OPr, H—BAB—OPr, H-(AB)₂—OPr or H-BA-OMe,
said process comprising the following steps:
   (i) a step of converting a compound of following formula T-A′-B′—Y, in particular T-A′-B′—OAll, or T-B′-A′-Y (I₀) into a donor compound of following formula T-A′-B′—X or T-B′-A′-X (I_{D}), by intermediately forming the hemiacetal T-A′-B′—OH or T-B′-A′-OH, wherein X represents a leaving group chosen from imidates, for example OPTFA or OTCA, PTFA representing N-phenyltrifluoroacetimidyl and TCA representing trichloroacetimidyl, from o-alkynylbenzoates, and from diphenyl oxosulfoniums, in particular, when Y is All, through metallo-catalyzed deallylation, for example Pd, Ir or Rh, more particularly in presence of H₂-activated Ir-catalyst, then aqueous I₂ or NIS, with optionally a base, in particular an inorganic base, for example NaHCO₃, or by Pd-catalyzed deallylation, in particular in presence of PdCl₂, then aqueous I₂ or NIS, with optionally a base, in particular an inorganic base, for example NaHCO₃, to provide the corresponding hemiacetal T-A′-B′—OH or T-B′-A′-OH and then PTFA-Cl or trichloroacetonitrile, and/or
   (ii) a step of converting a compound of following formula T-A′-B′—Y, in particular T-A′-B′—OAll, or T-B′-A′-Y (I₀) with T being not C₁-C₆ alkyl, into an acceptor compound of following formula H-A′-B′—Y, in particular H-A′-B′—OAll, or H—B′-A′-Y (I_{A}), in particular in presence of 2,3-dichloro-5,6-dicyano-1,4-benzoquinone (DDQ), CAN or an acid when T is Nap or PMB, or in presence of buffered TBAF, for example buffered with AcOH, or Et₃N·3HF, when T is a silyl, or in presence of an organic, inorganic, or Lewis acid, such as AcOH, TsOH, HCl, ZnBr₂ when T is THP, MEM, MOP, and/or
   (iii) a step of obtaining from compound (I_{A}) and/or (I_{D}) a compound Q′-(B′)ₓ-(A′-B′)ₘ-(A′)_{y}-Y, in particular Q′-(B′)ₓ-(A′-B′)ₘ-(A′)_{y}-OAll, or Q′-(A′)ₓ-(B′-A′)ₘ-(B′)_{y}—Y (II_{OP}), with m being from 1 to n, and Q′ being T when x is 0 and chosen from T, Bn and acyl groups, for example Lev, ClAc, Fmoc, or Ac when x is 1, in particular in presence of a Lewis acid, for example chosen from TMSOTf, TBSOTf, TfOH, Yb(OTf)₃, Cu(OTf)₂, AgOTf, or boron trifluoride etherate,
   (iv) when R is LZ and L is not —N(R^{a})-D-, a step of conjugating compound (II_{OP}) into a compound of following formula Q′-(B′)ₓ-(A′-B′)ₘ-(A′)_{y}-OLZ or Q′-(A′)ₓ-(B′-A′)ₘ-(B′)_{y}-OLZ (II_{CP}), or a compound of following formula Q′-(B′)ₓ-(A′-B′)ₘ-(A′)_{y}-OW or Q′-(A′)ₓ-(B′-A′)ₘ-(B′)_{y}—OW, wherein W is L-F₁′ or L-F₁′_{P}, L being as defined above, F₁′ being a precursor of F₁ as defined above, F₁′_{P} being a protected group F₁′, in particular with one or more benzyl groups,
   or when R is LZ and L is —N(R^{a})-D-, a step of preparation of a compound Q′-(B′)ₓ-(A′-B′)ₘ (A′)_{y}-OH or Q′-(A′)ₓ-(B′-A′)ₘ-(B′)_{y}—OH,
   (iv′) optionally, when m is not n, a step of converting a compound Q′-(B′)ₓ-(A′-B′)ₘ-(A′)_{y} OLZ or Q′-(A′)ₓ-(B′-A′)ₘ-(B′)_{y}-OLZ (II_{CP}), or a compound of following formula Q′-(B′)ₓ-(A′-B′)ₘ-(A′)_{y}-OW or Q′-(A′)ₓ-(B′-A′)ₘ-(B′)_{y}—OW to a compound Q′-(B′)ₓ-(A′-B′)ₙ-(A′)_{y}-OLZ or Q′-(A′)ₓ-(B′-A′)ₙ-(B′)_{y}-OLZ (II_{CP}), or a compound of following formula Q′-(B′)ₓ-(A′-B′)ₙ-(A′)_{y}-OW or Q′-(A′)ₓ-(B′-A′)ₙ-(B′)_{y}—OW respectively, being noted that when the Q′ group of Q′-(B′)ₓ-(A′-B′)ₘ-(A′)_{y}-OLZ or Q′-(A′)ₓ-(B′-A′)ₘ-(B′)_{y}-OLZ (II_{CP}) or Q′-(B′)ₓ-(A′-B′)ₘ-(A′)_{y}-OW or Q′-(A′)ₓ-(B′-A′)ₘ-(B′)_{y}—OW is not C₁-C₆ alkyl, the Q′ group of Q′-(B′)ₓ-(A′-B′)ₙ-(A′)_{y}-OLZ or Q′-(A′)ₓ-(B′-A′)ₙ-(B′)_{y}-OLZ (II_{CP}) or Q′-(B′)ₓ-(A′-B′)ₙ-(A′)_{y}-OW or Q′-(A′)ₓ-(B′-A′)ₙ-(B′)_{y}—OW can represent C₁-C₆ alkyl,
   (v) a step of deprotection of the compound obtained after step (iii) or (iv) to obtain the compound of following formula Q-(B)ₓ-(AB)ₙ-(A)_{y}-OLZ or Q-(A)ₓ-(BA)ₙ-(B)_{y}—OLZ (II) or a compound of following formula Q-(B)ₓ-(AB)ₙ-(A)_{y}-O-L-F₁′ or Q-(A)ₓ-(BA)ₙ-(B)_{y}—O-L-F₁′, or a compound of following formula Q-(B)ₓ-(AB)ₙ-(A)_{y}-OH or Q-(A)ₓ-(BA)ₙ-(B)_{y}—OH, in particular in presence of Pd(OH)₂—C or Pd—C, H₂, for example generated as high-pressure hydrogen with the electrolysis of water, and a base, in particular an inorganic base, for example chosen from NaHCO₃, K₂CO₃, NH₄HCO₃, CaCO₃, MgCO₃, and optionally followed by saponification then in presence of organic/inorganic base for example ethylenediamine, triethylamine, diethylamine, hydoxylamine, NH₂OH or of LiOH/H₂O₂, when R₁ is C₁-C₆ alkyl, notably Me, or before in presence of TBAF or TFA, ZnBr₂, TsOH, when T is a silyl ether, THP, MEM, MOP and/or P₁ is Boc,
   (vi) when the compound obtained in step (v) is of formula Q-(B)ₓ-(AB)ₙ-(A)_{y}-O-L-F₁′ or Q-(A)ₓ-(BA)ₙ-(B)_{y}—O-L-F₁′, a step of contacting said compound with
      a compound of following formula F₁″-L₂-Z₁, F₁″ being a precursor of F₁ as defined above, L₂ and Z₁ being as defined above, or
      a compound of following formula F₁″-L₂-F₂′, F₁″ being a precursor of F₁ as defined above, F₂′ being a precursor of F₂ as defined above, L₂ being as defined above, followed by contacting the obtained compound with a compound of following formula F₂″-L₃-Z₁, wherein F₂″ is a precursor of F₂ as defined above, and L₃ and Z₁ being as defined above, or when the compound obtained in step (v) is of formula Q-(B)ₓ-(AB)ₙ-(A)_{y}-OH or Q-(A)ₓ-(BA)ₙ-(B)_{y}—OH, a step of contacting said compound with:
      a compound of following formula HN(R^{a})-D-Z₁, R^{a}, D and Z₁ being as defined above, or
      a compound of following formula HN(R^{a})-D-F₁′, F₁′ being a precursor of F₁ as defined above, followed by contacting the obtained compound with a compound of following formula F₁″-L₂-Z₁, wherein F₁″ is a precursor of F₁ as defined above,
to give the compound of following formula Q-(B)ₓ-(AB)ₙ-(A)_{y}-OLZ or Q-(A)ₓ-(BA)ₙ-(B)_{y}-OLZ (II).

All the embodiments related to the conjugate, the immunogenic composition, the compounds of formula (IIa) or (IIb), or the use as defined above apply here as well, alone or in combination.

In a particular embodiment, the invention concerns a process of preparation of a compound of the following formula (IIa):

Q-(B)ₓ-(AB)ₙ-(A)_{y}-OR (IIa),

said process comprising the following steps:
(i) a step of converting a compound of following formula T-A′-B′—Y, in particular T-A′-B′—OAll, (I₀) into a donor compound of following formula T-A′-B′—X (I_{D}), by intermediately forming the hemiacetal T-A′-B′—OH, wherein X represents a leaving group chosen from imidates, for example OPTFA or OTCA, PTFA representing N-phenyltrifluoroacetimidyl and TCA representing trichloroacetimidyl, from o-alkynylbenzoates, and from diphenyl oxosulfoniums, in particular, when Y is All, through metallo-catalyzed deallylation, for example Pd, Ir or Rh, more particularly in presence of H₂-activated Ir-catalyst, then aqueous I₂ or NIS, with optionally a base, in particular an inorganic base, for example NaHCO₃, or by Pd-catalyzed deallylation, in particular in presence of PdCl₂, then aqueous I₂ or NIS, with optionally a base, in particular an inorganic base, for example NaHCO₃, to provide the corresponding hemiacetal T-A′-B′—OH and then PTFA-Cl or trichloroacetonitrile,
   and/or
(ii) a step of converting a compound of following formula T-A′-B′—Y, in particular T-A′-B′—OAll (I₀) with T being not C₁-C₆ alkyl, into an acceptor compound of following formula H-A′-B′—Y, in particular H-A′-B′—OAll, (I_{A}), in particular in presence of 2,3-dichloro-5,6-dicyano-1,4-benzoquinone (DDQ), CAN or an acid when T is Nap or PMB, or in presence of buffered TBAF, for example buffered with AcOH, or Et₃N·3HF, when T is a silyl, or in presence of organic/inorganic/Lewis acid such as AcOH, TsOH, HCl, ZnBr₂ when T is THP, MEM, MOP,
   and/or
(iii) a step of obtaining from compound (I_{A}) and/or (I_{D}) a compound Q′-(B′)ₓ-(A′-B′)ₘ (A′)_{y}-Y, in particular Q′-(B′)ₓ-(A′-B′)ₘ-(A′)_{y}-OAll, (II_{OP}), with m being from 1 to n, and Q′ being T when x is 0 and chosen from T, Bn and acyl groups, for example Lev, ClAc, Fmoc, or Ac when x is 1, in particular in presence of a Lewis acid, for example chosen from TMSOTf, TBSOTf, TfOH, Yb(OTf)₃, Cu(OTf)₂, AgOTf, or boron trifluoride etherate,
(iv) when R is LZ, a step of conjugating compound (II_{OP}) into a compound of following formula Q′-(B′)ₓ-(A′-B′)ₘ-(A′)_{y}-OLZ (II_{CP}), or a compound of following formula Q′-(B′)ₓ-(A′-B′)ₘ-(A′)_{y}-OW, wherein W is L-F₁′ or L-F₁′_{P}, L being as defined above, F₁′ being a precursor of F₁ as defined above, F₁′_{P} being a protected group F₁′, in particular with one or more benzyl groups,
(iv′) optionally, when m is not n, a step of converting a compound Q′-(B′)ₓ-(A′-B′)ₘ-(A′)_{y} OLZ (II_{CP}), or a compound of following formula Q′-(B′)ₓ-(A′-B′)ₘ-(A′)_{y}-OW to a compound Q′-(B′)ₓ-(A′-B′)ₙ-(A′)_{y}-OLZ (II_{CP}), or a compound of following formula Q′-(B′)ₓ-(A′-B′)ₙ-(A′)_{y}-OW respectively, being noted that when the Q′ group of Q′-(B′)ₓ-(A′-B′)ₘ-(A′)_{y}-OLZ (II_{CP}) or Q′-(B′)ₓ-(A′-B′)ₘ-(A′)_{y}-OW is not C₁-C₆ alkyl, the Q′ group of Q′-(B′)ₓ-(A′-B′)ₙ-(A′)_{y}-OLZ (II_{CP}) or Q′-(B′)ₓ-(A′-B′)ₙ-(A′)_{y}-OW can represent C₁-C₆ alkyl,
(v) a step of deprotection of the compound obtained after step (iii) or (iv) to obtain the compound of following formula Q-(B)ₓ-(AB)ₙ-(A)_{y}-OLZ or Q-(A)ₓ-(BA)ₙ-(B)_{y}—OLZ (II) or a compound of following formula Q-(B)ₓ-(AB)ₙ-(A)_{y}-O-L-F₁′ or Q-(A)ₓ-(BA)ₙ-(B)_{y}—O-L-F₁′, in particular in presence of Pd(OH)₂—C or Pd—C, H₂, for example generated as high-pressure hydrogen with the electrolysis of water, and a base, in particular an inorganic base, for example chosen from NaHCO₃, K₂CO₃, NH₄HCO₃, CaCO₃, MgCO₃, and optionally then in presence of NH₂OH or NH₄OH, said hydrogenation being optionally followed by saponification in presence of organic or inorganic base for example ethylenediamine, triethylamine, diethylamine, hydoxylamine, NH₂OH or of LiOH/H₂O₂, when R₁ is C₁-C₆ alkyl, notably Me, or before in presence of TBAF or TFA, ZnBr₂, TsOH, when T is a silyl ether, THP, MEM, MOP and/or P₁ is Boc,
(vi) when the compound obtained in step (v) is of formula Q-(B)ₓ-(AB)ₙ-(A)_{y}-O-L-F₁′ or Q-(A)ₓ-(BA)ₙ-(B)_{y}—O-L-F₁′, a step of contacting said compound with
   a compound of following formula F₁″-L₂-Z₁, F₁″ being a precursor of F₁ as defined above, L₂ and Z₁ being as defined above, or
   a compound of following formula F₁″-L₂-F₂′, F₁″ being a precursor of F₁ as defined above, F₂′ being a precursor of F₂ as defined above, L₂ being as defined above, followed by contacting the obtained compound with a compound of following formula F₂″-L₃-Z₁, wherein F₂″ is a precursor of F₂ as defined above, and L₃ and Z₁ being as defined above,
      to give the compound of following formula Q-(B)ₓ-(AB)ₙ-(A)_{y}-OLZ or Q-(A)ₓ-(BA)ₙ-(B)_{y}-OLZ (II).

F₁′ and F₁″ are for example:
—C(═O)—C(═O)H and an amine, forming a —C(═O)—C(═O)—NH— group,
—C(═O)—C(═O)H and an amine, forming a —C(═O)—C(H)═N—NH— group,
An carboxylic acid or an activated ester and an alcohol, forming an ester,
an activated ester and an amine, forming an amide,
an amine and a leaving group, forming an amine,
a thiol and a leaving group, forming thioether,
an amine and an aldehyde, forming an imine,
an amine and an aldehyde, forming an amine, in particular by reductive amination,
a thiol and a maleimide, forming a thio-succinimide,
an azide and an alkyne, forming a triazole, in particular by click chemistry,
C(═O)CH₂-Hal, in particular Br, and an amine, forming a C(═O)CH₂—NH group,
NHC(═O)CH₂-Hal, in particular Br, and an amine, forming a NHC(═O)CH₂—NH group,
C(═O)CH₂-Hal, in particular Br, and a thiol, forming a C(═O)CH₂—SH group,
NHC(═O)CH₂-Hal, in particular Br, and a thiol, forming a NHC(═O)CH₂—SH group,
a

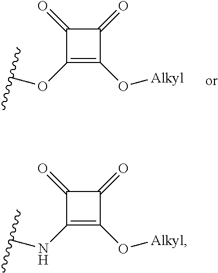

and an amine or a alcohol, forming

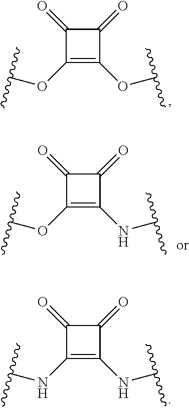 [SMILES: *Oc1c(O*)c(=O)c1=O ; *Nc1c(O*)c(=O)c1=O ; *Nc1c(N*)c(=O)c1=O]

F₂′ and F₂′ are for example:
—C(═O)—C(═O)H and an amine, forming a —C(═O)—C(═O)—NH— group,
—C(═O)—C(═O)H and an amine, forming a —C(═O)—C(H)═N—NH— group,
An carboxylic acid or an activated ester and an alcohol, forming an ester,
an activated ester and an amine, forming an amide,
an amine and a leaving group, forming an amine,
a thiol and a leaving group, forming thioether,
an amine and an aldehyde, forming an imine,
an amine and an aldehyde, forming an amine, in particular by reductive amination,
a thiol and a maleimide, forming a thio-succinimide,
an azide and an alkyne, forming a triazole, in particular by click chemistry,
C(═O)CH₂-Hal, in particular Br, and an amine, forming a C(═O)CH₂—NH group,
NHC(═O)CH₂-Hal, in particular Br, and an amine, forming a NHC(═O)CH₂—NH group,
C(═O)CH₂-Hal, in particular Br, and a thiol, forming a C(═O)CH₂—SH group,
NHC(═O)CH₂-Hal, in particular Br, and a thiol, forming a NHC(═O)CH₂—SH group,
a

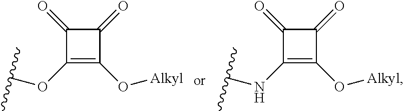

and an amine or a alcohol, forming

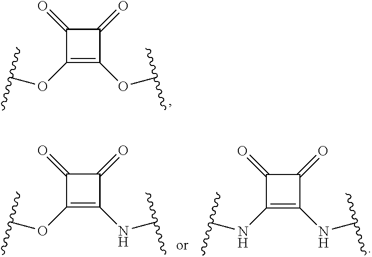 [SMILES: *Oc1c(O*)c(=O)c1=O ; *Nc1c(O*)c(=O)c1=O ; *Nc1c(N*)c(=O)c1=O]

Reference is also made regarding glyoxylyl group to Bioconjugate Chem. 2013, 24, 735-765

In a particular embodiment, F₁″ and F₂′ are orthogonal (i.e. in particular they do react together).

Step (i) can be performed in two substeps.

When Y is OAll, the first substep is in particular an anomeric deallylation well known from the skilled in the art, more particularly metallo-catalyzed deallylation, the metal being for example Pd, Ir or Rh, more particularly in presence of H₂-activated Ir-catalyst or a pallado-catalyzed anomeric deallylation, notably in presence of PdCl₂, notably followed by cleavage that can be iodine-assisted.

The deallylation can also be performed as described in Carbohydrate Research 342 (2007) 2635-2640, for example in presence of DABCO and (Ph₃P)₃RhCl, followed by mercuric-assisted cleavage.

When Y is OPMP, deprotection can be performed by any procedure well known from the skilled in the art, for example using CAN (reference is for instance made to Synthesis 2018, 50, 4270-4282).

When Y is SR₄, conversion to the donor can be performed by methods well known from the skilled in the art. Reference is for example made to Carbohydrate Research 403 (2015) 13-22. Suitable promoters are those capable of generating thiophilic species, which can in particular be categorized into four major types: (1) metal salts; (2) halonium reagents; (3) organosulfur reagents; (4) single electron transfer (SET) reagents/methods. Widely used examples are NIS/HOTf or NIS/AgOTf. Organosulfur reagents constitute another widely used group of promoters for glycosidation of thioglycosides, for example Dimethyl(thiomethyl)sulfonium triflate (DMTST). Also powerful promoters are the combination of sulfinyl derivatives and Tf₂O.

The second substep is in particular an activation of the obtained hemiacetal into an imidate donor (anomeric OPTFA or OTCA substitution) (J. Org. Chem. (2015) 80, 11237-57), or into a alkynyl benzoate donor (Acc. Chem. Res. 2018, 51, 507-516), or into a diphenyl oxo sulfoniums (J. Am. Chem. Soc. 2000, 122, 4269-4279).

Other possible glycosylation methods are provided in the Handbook of Chemical GlycosylationAdvances in Stereoselectivity and Therapeutic Relevance (2008), Wiley, A. V. Demchenko.

Step (ii) is the cleavage (deprotection) of the T group, with T being not C₁-C₆ alkyl, in particular a Nap, PMB or silyl deprotection well known from the skilled in the art.

For example, the Nap or PMB protecting group can be removed by in presence of 2,3-dichloro-5,6-dicyano-1,4-benzoquinone (DDQ), CAN or an acid such as TFA (Mulard et al., J. of Organic Chemistry, doi: 10.1021/acs.joc.0c00777; Chem. Commun., 2014, 50, 3155) or HCl in hexafluoro-iso-propanol (HFIP) (J. Org. Chem. 2015, 80, 8796-8806).

Silyl ethers can for example be cleaved in presence of buffered TBAF, for example buffered with AcOH, or Et₃N·3HF. Other methods well know from those skilled in the art can be found in Nelson et al. (Synthesis 1996; 1996(9): 1031-1069).

Step (iii) can be performed in presence of a Lewis acid, for example chosen from TMSOTf, TBSOTf, TfOH, boron trifluoride etherate, or Lewis acidic metal salts (JACS (2015) 137, 12653).

A large diversity of other glycosylation promoters are described in Angew Chem (2009) 48, 1900-34.

Other possible glycosylation promoters are provided in the Handbook of Chemical Glycosylation Advances in Stereoselectivity and Therapeutic Relevance (2008), Wiley, A. V. Demchenko.

Step (iv) can be performed in three substeps.

The first substep is in particular an anomeric deallylation well known from the skilled in the art, more particularly a pallado-catalyzed anomeric deallylation or a deallylation in presence of H₂-activated Ir-catalyst.

The second substep is in particular an activation of the obtained hemiacetal into an imidate donor (anomeric OPTFA or OTCA substitution) (J. Org. Chem. (2015) 80, 11237-57).

The third step is the reaction of the activated compound obtained in previous step with a compound such as HO-LZ or HO—W, wherein W is L-F₁′ or L-F₁′_{P}.

Step (v) comprises a deprotection by hydrogenation. This hydrogenation can be performed by conventional methods known from the skilled in the art, but also by using dihydrogen generated with the electrolysis of water, notably as high-pressure hydrogen, for example thanks to a H-Cube system.

The base is in particular present in a quantity ranging from 1 equivalent with reference to the starting material to 1 equivalent per chlorine present in the compound subjected to step (v).

More particularly, the base is in particular present in a quantity ranging from ⅓ per chlorine present in the compound subjected to step (v) to 1 equivalent per chlorine present in the compound subjected to step (v).

In a preferred embodiment, the base is not present at the beginning of the deprotection reaction, but added afterwards, preferably once all the Bn, Bzl and Nap have been cleaved, and/or portionwise.

In another preferred embodiment, the base is present at the beginning of the deprotection reaction, in a ⅓ equivalent with respect to the starting material, and then further added afterwards, preferably once all the Bn, Bzl and Nap have been cleaved, and/or portionwise.

This hydrogenation can be preceded or followed by another deprotection step, in particular when a silyl ether (for example OTBS), Ac, and/or Boc group is present, as well known from the skilled in the art.

When R₁ is C₁-C₆ alkyl, notably Me, said hydrogenation can be preceded by a saponification step using for example LiOH/H₂O₂ or other milder methods well known from the skilled in the art. An example of suitable procedure can be found in Org. Biomol. Chem., 2013, 42, 3510.

When x is 1 and Q′ is chosen from acyl groups, for example Lev, ClAc, Fmoc, or Ac, said hydrogenation can be preceded or followed by a cleavage by acid methanolysis or with a mild base).

When P₁ is Alloc, Alloc is preferably removed to give deprotected —NH₂ and then converted into —NHAC before said hydrogenation step.

When R₂ is CH₂OR₃, with R₃ being Ac, the CH₂OR₃ is preferably converted prior to step (iv), (iv′) or (v) to a CH₂OH group, for example in presence of NaOMe, and then to a CO₂Bn group using for instance a) TBABr, NaHCO₃, TEMPO, NaOCl; b) HCl, 2-methylbut-2-ene, NaClO₂, NaH₂PO₄; c) CsF, BnBr. An example of such a procedure can be found in Chem Eur J (2010) 16, 3476.

When P₁ and P₂ form together a tetrachlorophthalimido (Cl₄Phth) group, deprotection can be performed before the hydrogenation step using for example H₂NCH₂CH₂NH₂ and then Ac₂O.

When P₁ and P₂ form together a phthalimido group, deprotection can be performed before the hydrogenation step, and in particular before the conversion of CH₂OR₃ to CO₂Bn when R₂ is CH₂OR₃, by methods well known from the skilled in the art. Step (vi) is the formation of the F₁ group from the F₁′ residue and the F₁″-L₂-Z₁ compound, or from the residue F₁′ and the compound of following formula F₁″-L₂-F₂′, followed by contacting the obtained compound with a compound of following formula F₂″-L₃-Z₁.

This can be done by any method known by the skilled in the art, for instance by bioconjugation methods, in particular when Z₁ is a biomolecule. Such methods are notably described in Bioconjugate Techniques: Third Edition (2013), Greg Hermanson, Elsevier.

For example, it can be done by amide formation, reductive amination, oxime formation, hydrazone formation, or by thiol-ene reaction, using for instance an alkene or vinylphophonothiolate. Examples of methods of oxime and hydrazine litigation have been described in Chem. Eur. J. 2014, 20, 34-41, and Chem. Rev. 2017, 117, 10358-10376. Example of chemically induced vinylphosphonothiolate electrophiles for Thiol-Thiol bioconjugation are described (J. Am. Chem. Soc. 2020, 142, 9544-9552).

In a particular embodiment, step (iii) is a step of obtaining, from compound (I_{A}) and a compound Q′-B′—X, a compound Q′-B′-(A′-B′)ₘ—Y, in particular Q′-B′-(A′-B′)ₘ-OAll (II_{OP}), with Q′ being as defined above.

In a particular embodiment, step (iii) is a step of obtaining, from compound (I_{D}) and a compound H-A′-Y, in particular H-A′-OAll, a compound T-(A′-B′)ₘ-A′-Y, in particular T-(A′-B′)ₘ-A′-OAll (II_{OP}), with T being as defined above.

In a particular embodiment, step (iii) is a step of obtaining from compound (I_{A}) and (I_{D}) a compound T-(A′-B′)₂—Y, in particular T-(A′-B′)₂—OAll, said compound T-(A′-B′)₂—Y, in particular T-(A′-B′)₂—OAll (II_{OP}) being possibly:
a) further converted into a compound T-(A′-B′)₂—X, and then reacted with a compound H-A′-B′—Y, in particular H-A′-B′—OAll (I_{A}), or
b) further converted into a compound T-(A′-B′)₂—X, and then reacted with a compound H-(A′-B′)₂—Y, in particular H-(A′-B′)₂—OAll obtained from T-(A′-B′)₂—Y, in particular T-(A′-B′)₂—OAll, in particular in presence of 2,3-dichloro-5,6-dicyano-1,4-benzoquinone (DDQ), CAN or acid when T is Nap or PMB, or in presence of Et₃N·3HF or AcOH buffered TBAF when T is a silyl, or
c) when T is not C₁-C₆ alkyl, further converted into a compound H-(A′-B′)₂—Y, in particular H-(A′-B′)₂—OAll, and then reacted with a compound T-A′-B′—X (I_{D}), or
d) when T is not C₁-C₆ alkyl, further converted into a compound H-(A′-B′)₂—Y, in particular H-(A′-B′)₂—OAll, and then reacted with a compound T-(A′-B′)₂—X,
e) at least one of the steps a) to d) being if necessary repeated with at least one of the compounds obtained in the previous steps;
   to yield a compound of formula Q′-(B′)ₓ-(A′-B′)ₘ-(A′)_{y}-Y, in particular Q′-(B′)ₓ-(A′-B′)ₘ (A′)_{y}-OAll (II_{OP}) when m is 3 or more.

In fact, step (iii) can be performed in several substeps, which consist in the conversion of a starting material or a compound obtained in a previous step into an acceptor or donor, followed by a reaction with a donor or acceptor respectively, obtained as specified above, until the desired length of oligosaccharide is obtained.

For example, the desired length of oligosaccharide (corresponding to a compound with the target n value) can be obtained by forming intermediately a T-A′-B′—X, T-(A′-B′)₂—X,T-(A′-B′)₃—X or even T-(A′-B′)₄—X donor, and/or a H-A′-B′—Y, H-(A′-B′)₂Y, H-(A′-B′)₃—Y or even H-(A′-B′)₄—Y acceptor.

When Q′ is C₁-C₆ alkyl, said Q′ is for example introduced via a donor compound wherein Q′ or T is C₁-C₆ alkyl during step (iii) or (iv′), being noted that the obtained compound cannot be converted into an acceptor, but again into a donor to be reacted with an acceptor and optionally again converted into a donor until the desired value of m or n is achieved.

In a particular embodiment, when R is LZ, step (iv) comprises the following substeps:
a) a substep of deprotection of compound (II_{OP}) to obtain a hemiacetal compound of following formula Q′-(B′)ₓ-(A′-B′)ₘ-(A′)_{y}-OH,
b) a substep of activation of the hemiacetal compound of following formula Q′-(B′)ₓ-(A′-B′)ₘ-(A′)_{y}-OH to obtain a compound of following formula Q′-(B′)ₓ-(A′-B′)ₘ-(A′)_{y}-X′, wherein X′ represents an imidate, for example OPTFA or OTCA, PTFA representing N-phenyltrifluoroacetimidyl and TCA representing trichloroacetimidyl,
c) a substep of reaction of compound of following formula Q′-(B′)ₓ-(A′-B′)ₘ-(A′)_{y}-X′ with a compound of formula HO-LZ, for example an alcohol of formula HO—C₁-C₁₂ alkyl such as methanol; or with a compound of formula HO-L-F₁′ or HO-L-F₁′_{P}, to give the compound Q′-(B′)ₓ-(A′-B′)ₘ-(A′)_{y}-OW (II_{CP}), wherein W is L-F₁′ or L-F₁′_{P}as defined above.

When Z₁ is protected, Z₁ may be deprotected, for example in a last step of deprotection.

In a particular embodiment, L is a divalent C₁-C₁₂ alkyl or alkenyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, and F₁′_{P} is a N₃, NHCbz, or NBnCbz group, L-F₁′_{P} being notably a PEG chain bearing a N₃, NHCbz, NBnCbz, or SBn group (for this latter, see for example Chem. Sci. 2014, 5, 1992).

In a particular embodiment, the F₁′ or F₁′_{P} group reacts with F₁″-L₂-Z₁ which is a compound bearing a first reactive function that will react with the F₁′ or F₁′_{P} residue to form the F₁ function. In a particular embodiment, the F₁′ or F₁′_{P} group reacts with F₁″-L₂-F₂′ that further comprises a second reactive function F₂′, which is orthogonal to the first reactive function F₁″.

In a more particular embodiment, HO-L-Z₁ or HO-L-F₁′_{P} is HO—(CH₂)ₚ—N₃, or HO—(CH₂)ₚ—NHCbz, HO—(CH₂)ₚ-NBnCbz, L-Z₁ or L-F₁′ is —(CH₂)ₚ—NH₃⁺ or —(CH₂)ₚ—NH₂, wherein p ranges from 1 to 10, in particular from 2 to 8, more particularly 2, 3, 4, 5 or 6, and F₁″-L₂-Z₁ or F₁″-L₂-F₂′ is an activated version, in particular an activated ester of the compound of the following formula:

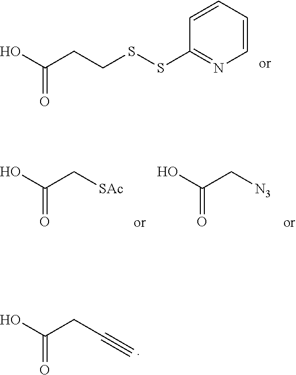

In another more particular embodiment, HO-L-Z₁ or HO-L-F₁′_{P} is HO—(CH₂)ₚ—CH₂═CH₂, and F₁″-L₂-Z₁ is a thiol, for example HS-Bn. Reference is in particular made to Angew. Chem. Int. Ed. 2014, 53, 3894-3898.

In another more particular embodiment, HO-L-Z₁ or HO-L-F₁′_{P} is HO—(CH₂)ₚ-SBn, L-Z₁ or L-F₁′_{P} is —(CH₂)ₚ—SBn, or —(CH₂)ₚ—SO₂H (reference is in particular made for this latter case to Chem. Eur. J. 2004, 10, 4265-4282), deprotected L-Z₁ or L-F₁′ is —(CH₂)ₚ—SH, wherein p ranges from 1 to 10, in particular from 2 to 8, more particularly 2, 3, 4, 5 or 6. In a particular embodiment, HO-L-Z₁ or HO-L-F₁′_{P} is a HO—C₁-C₁₀-alkyl wherein a —CH₂— is replaced by a hemiacetal or an acetal, in particular an optionally substituted cyclic acetal, for example 2-methyl-1,3-dioxolane-2-ethanol.

In a particular embodiment, HO-L-Z₁ or HO-L-F₁′_{P} is HO—(CH₂)ₚ-OBn, and deprotected L-Z₁ or L-F₁′ is —(CH₂)ₚ—OH, wherein p ranges from 1 to 10, in particular from 2 to 8, more particularly 2, 3, 4, 5 or 6. The introduced primary alcohol may for example be converted into an aldehyde moiety upon selective oxidation, and then react with a F₁″-L₂-Z₁ or F₁″-L₂-F₂′ compound comprising a hydrazide-, an oxime- or a derivative. F₁″-L₂-Z₁ or F₁″-L₂-F₂′ optionally further comprises a second reactive function, which is orthogonal to the first reactive function, and may be selected from alkene, alkyne and masked thiol groups.

In a particular embodiment, when HO-L-Z₁ or HO-L-F₁′_{P} is HO—CH₂—C(OBn)-CH₂—OBn, L-Z₁ or L-F₁′ is —CH₂—C(OH)—CH₂—OH, and F₁″-L₂-Z₁ or F₁″-L₂-F₂′ is of the following formula:

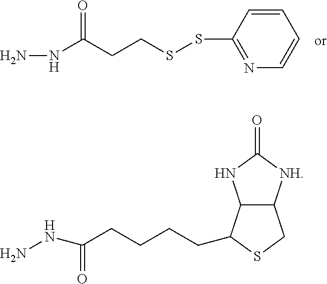 [SMILES: NNC(=O)CCSSc1ccccn1 ; NNC(=O)CCCCC1SCC2NC(=O)NC21]

When T is C₁-C₆ alkyl, the C₁-C₆ alkyl may also be introduced after deprotection of a T protecting group, for example by contacting the deprotected compound with a C₁-C₆ alkyl-leaving group compound. Suitable leaving groups are known from the skilled in the art.

In a particular embodiment, compound T-A′-B′—OAll (I₀), with T=Nap, is obtained from the following compound, in particular in presence of TEMPO and BAIB, and then of BnBr and an optional base, for example an inorganic base such as K₂CO₃:

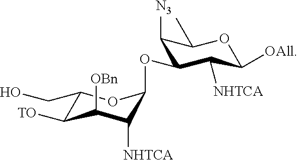

In a particular embodiment, the following compound:

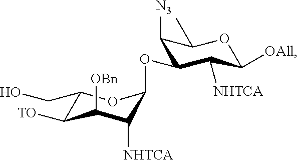

with T=Nap,
is obtained from the following compound, in particular in presence of TBAF:

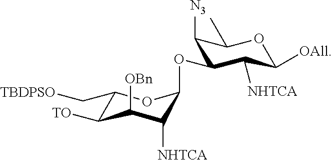

In a particular embodiment, the following compound:

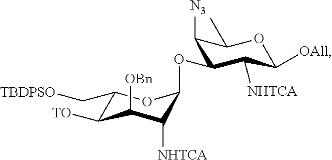

with T=Nap,
is obtained from the following compound:

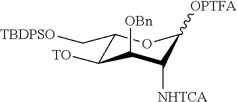

in particular in presence of a Lewis acid, for example chosen from TMSOTf, TBSOTf, TfOH and boron trifluoride etherate, and the following compound:

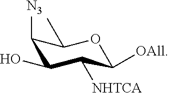

A detailed procedure has been for example described by H. B. Pfister, and L. A. Mulard, (Org. Lett. 2014, 16, 4892-4895).

In a particular embodiment, the following compound:

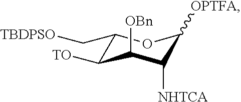

with T=Nap, is obtained from the following compound:

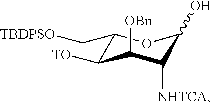

in particular in presence of PTFA-C₁, and a base, for example an inorganic base such as Cs₂CO₃.

In a particular embodiment, the following compound:

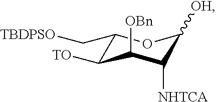

with T=Nap,
is obtained from the following compound:

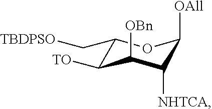

in particular in presence of H₂-activated Ir-catalyst or by Pd-catalyzed deallylation, in particular in presence of PdCl₂, and then aqueous I₂ or NIS, with optionally a base, in particular an inorganic base, for example NaHCO₃,
said compound

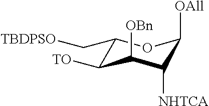

being preferably obtained from the following compound:

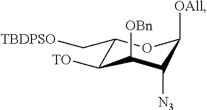

in particular in presence of PPh₃ or Zn and optionally AcOH, and then trichloroacetonitrile, with optionally a base, in particular an organic base such as Et₃N,
said compound

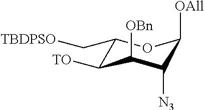

being more preferably obtained from the following compound:

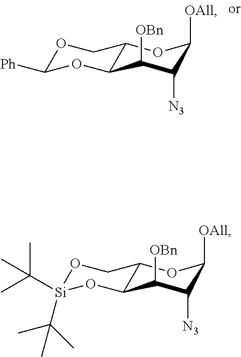

in particular in presence of methanol and CSA, and then TBDPS-Cl, with a base, in particular an organic base, for example imidazole, and then 2-(bromomethyl)naphthalene with a strong base, in particular NaH.

In a particular embodiment, compound T-A′-B′—OAll (I₀), with T=TBS, is obtained from the following compound, in particular in presence of TBSO-W wherein W is a leaving group in particular chosen from halogens and Tf, in particular TBSOTf and a base, in particular an organic base, for example imidazole:

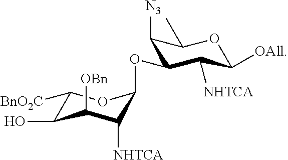

In a particular embodiment, the following compound:

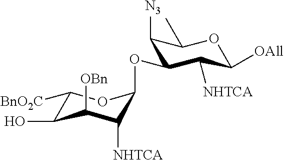

is obtained from the following compound, as for example described by H. B. Pfister and L. A. Mulard, (Org. Lett. 2014, 16, 4892-4895):

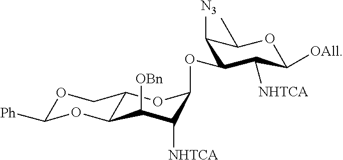

In a particular embodiment, compound T-A′-B′—OAll (I₀), with T=C₁-C₆ alkyl, is obtained from the following compound, in particular in presence of TEMPO and BAIB, and then of BnBr and optionally a base, for example an inorganic base such as K₂CO₃:

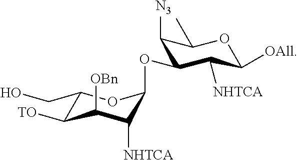

In a particular embodiment, the following compound:

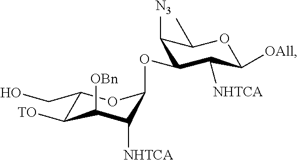

with T=C₁-C₆ alkyl,
is obtained from the following compound, in particular in presence of a Lewis acid, for example chosen from TMSOTf, TBSOTf, TfOH and boron trifluoride etherate, and then TBAF:

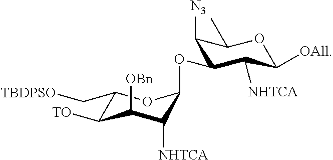

In a particular embodiment, the following compound:

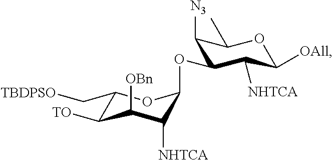

with T=C₁-C₆ alkyl,
is obtained from the following compound,

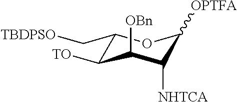

in particular in presence of a Lewis acid, for example chosen from TMSOTf, TBSOTf, TfOH and boron trifluoride etherate, and the following compound:

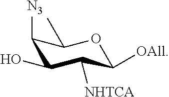

In a particular embodiment, the following compound:

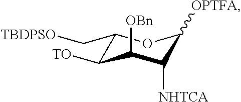

with T=C₁-C₆ alkyl,
is obtained from the following compound,

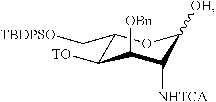

in particular in presence of PTFA-C₁, and optionally a base, for example an inorganic base such as Cs₂CO₃.

In a particular embodiment, the following compound:

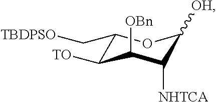

with T=C₁-C₆ alkyl,
is obtained from the following compound,

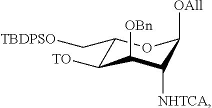

in particular in presence of H₂-activated Ir-catalyst, then aqueous I₂ or NIS, with optionally a base, in particular an inorganic base, for example NaHCO₃, or by Pd-catalyzed deallylation, in particular in presence of PdCl₂, then aqueous I₂ or NIS, with optionally a base, in particular an inorganic base, for example NaHCO₃,
said compound

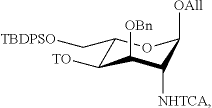

being preferably obtained from the following compound:

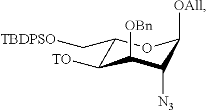

in particular in presence of Zn and optionally AcOH, and then trichloroacetonitrile, with optionally a base, in particular an organic base such as Et₃N,
said compound

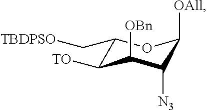

being more preferably obtained from the following compound:

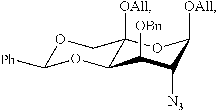

in particular in presence of metanol and CSA, and then ^{t}BDPS-Cl, with a base, in particular an organic base, for example imidazole, and then T-I with a strong base such as NaH.

In another aspect, the invention concerns a compound of one of the following formulae (III):

Q′-(B′)ₓ-(A′-B′)ₘ-(A′)_{y}-Y or Q′-(B′)ₓ-(A′-B′)ₘ-(A′)_{y}-OLZ,Q′-(B′)ₓ-(A′-B′)ₘ-(A′)_{y}-O-L-F₁′,Q′-(B′)ₓ-(A′-B′)ₘ(A′)_{y}-O-L-F₁′_{P},Q′-(B′)ₓ-(A′-B′)ₘ(A′)_{y}-OH,H—(B′)ₓ-(A′-B′)ₘ-(A′)_{y}Y,Q′-(A′)ₓ-(B′-A′)ₘ(B′)_{y}—Y or Q′-(A′)ₓ-(B′-A′)ₘ-(B′)_{y}-OLZ,Q′-(A′)ₓ-(B′-A′)ₘ-(B′)_{y}—O-L-F₁′,Q′-(A′)ₓ-(B′-A′)ₘ-(B′)_{y}—O-L-F₁′_{P},Q′-(A′)ₓ-(B′-A′)ₘ-(B′)_{y}—OH,H-(A′)ₓ-(B′-A′)ₘ-(B′)_{y}—Y,

Wherein Q′, A′, B′, Y, L, Z, F₁′, F₁′_{P}, x and m are as defined above,
With the proviso that:
When the compound is H—(B′)ₓ-(A′-B′)ₘ-(A′)_{y}-Y, in particular H—(B′)ₓ-(A′-B′)ₘ-(A′)_{y} OAll, and x=y=0, then m ranges from 3 to 50, in particular from 4 or 5 to 50.

In another aspect, the invention concerns a compound of one of the following formulae (III):

Q′-(B′)ₓ-(A′-B′)ₘ-(A′)_{y}-Y or Q′-(B′)ₓ-(A′-B′)ₘ-(A′)_{y}-OLZ,Q′-(B′)ₓ-(A′-B′)ₘ-(A′)_{y}-O-L-F₁′,Q′-(B′)ₓ-(A′-B′)ₘ-(A′)_{y}-O-L-F₁′_{P},Q′-(B′)ₓ-(A′-B′)ₘ-(A′)_{y}-OH,H—(B′)ₓ-(A′-B′)ₘ-(A′)_{y}Y,Q′-(A′)ₓ-(B′-A′)ₘ-(B′)_{y}—Y or Q′-(A′)ₓ-(B′-A′)ₘ-(B′)_{y}-OLZ,Q′-(A′)ₓ-(B′-A′)ₘ-(B′)_{y}—O-L-F₁′,Q′-(A′)ₓ-(B′-A′)ₘ-(B′)_{y}—O-L-F₁′_{P},Q′-(A′)ₓ-(B′-A′)ₘ-(B′)_{y}—OH,H-(A′)ₓ-(B′-A′)ₘ-(B′)_{y}—Y,

wherein Q′, A′, B′, Y, L, Z, F₁′, F₁′_{P}, x and m are as defined above,
with the proviso that:
When the compound is H—(B′)ₓ-(A′-B′)ₘ-(A′)_{y}-Y, in particular H—(B′)ₓ-(A′-B′)ₘ-(A′)_{y} OAll, and x=y=0, then m ranges from 3 to 50, in particular from 4 or 5 to 50;
When the compound is Q′-(B′)ₓ-(A′-B′)ₘ-(A′)_{y}-Y or Q′-(A′)ₓ-(B′-A′)ₘ-(B′)_{y}—Y, in particular Q′-(B′)ₓ-(A′-B′)ₘ(A′)_{y}-OAll or Q′-(A′)ₓ-(B′-A′)ₘ(B′)_{y}-OAll, and x+y=1, then m ranges from 2 to 50, in particular from 3, 4 or 5 to 50.

All the embodiments related to the conjugate, the immunogenic composition, the compounds of formula (IIa) or (IIb), the use or the process as defined above apply here as well, alone or in combination.

In a particular embodiment, said compound is not a compound wherein A′ is

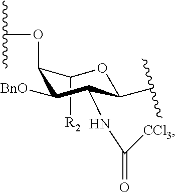

### and B′ is

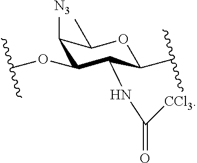

In a particular embodiment, when A′ is

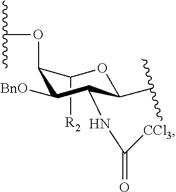

### and B′ is

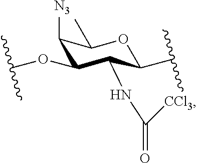

then T is not a silyl, in particular TBS.

In a particular embodiment, when A′ is

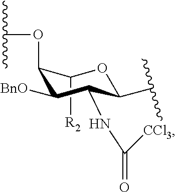

### and B′ is

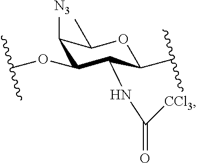

then T is Nap.
In part

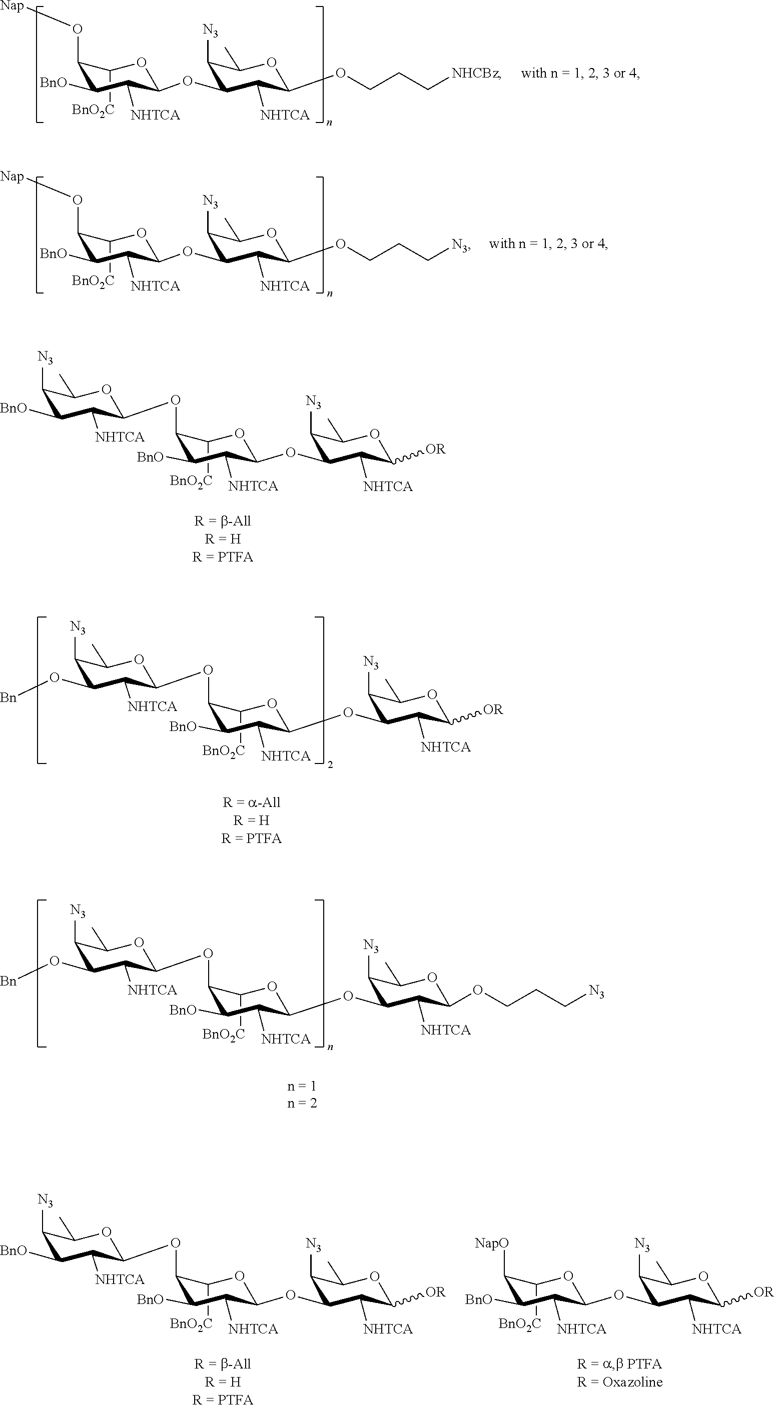

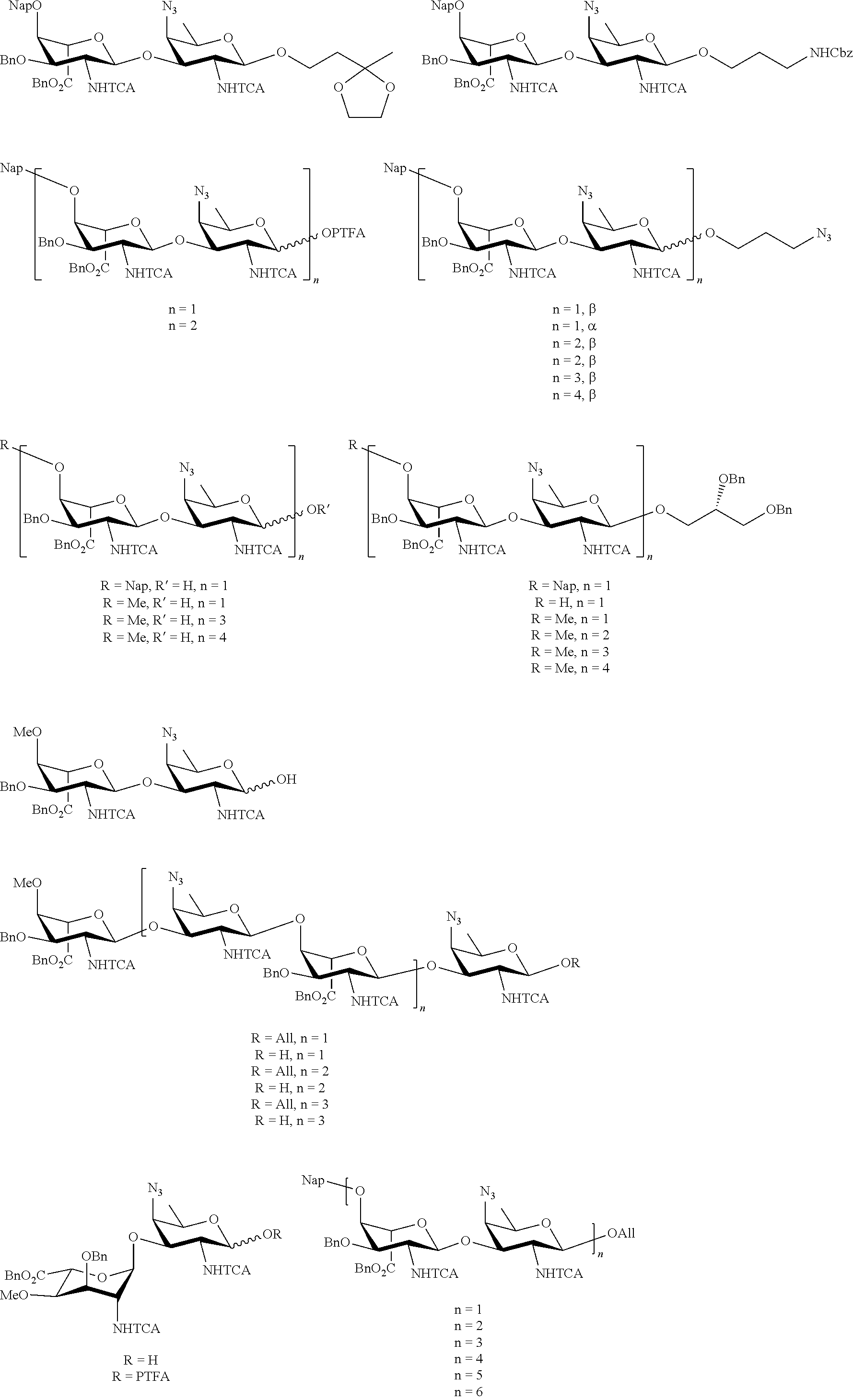

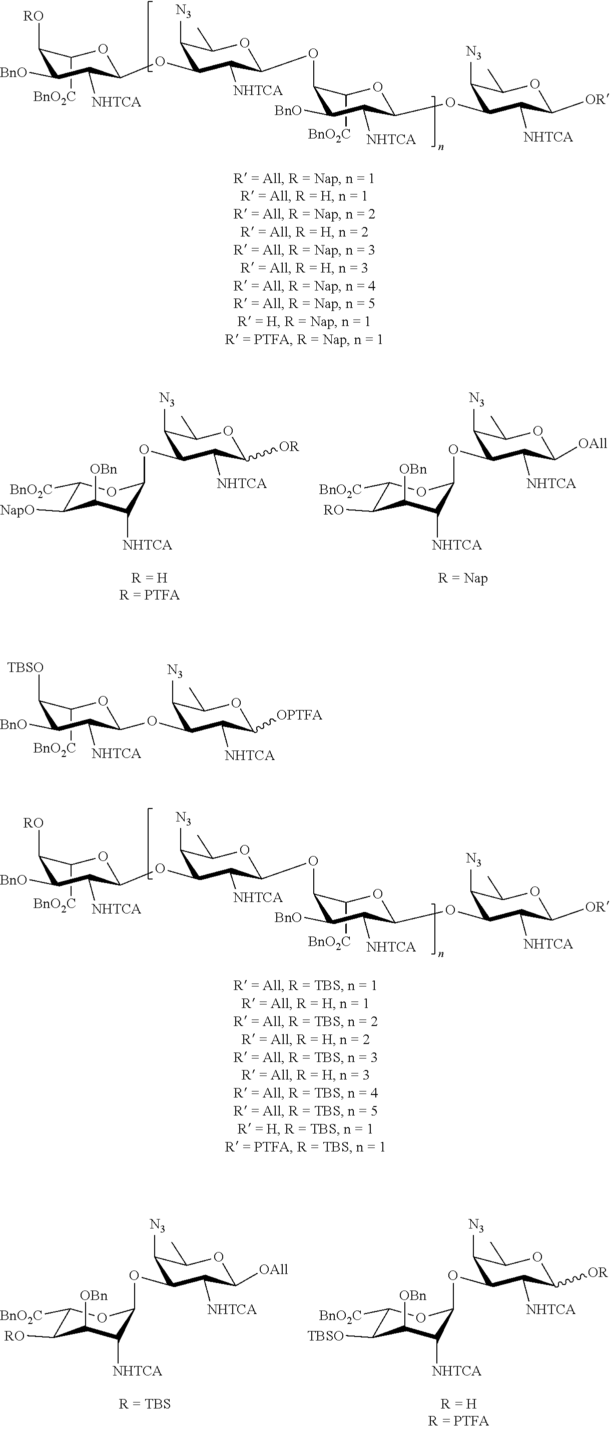

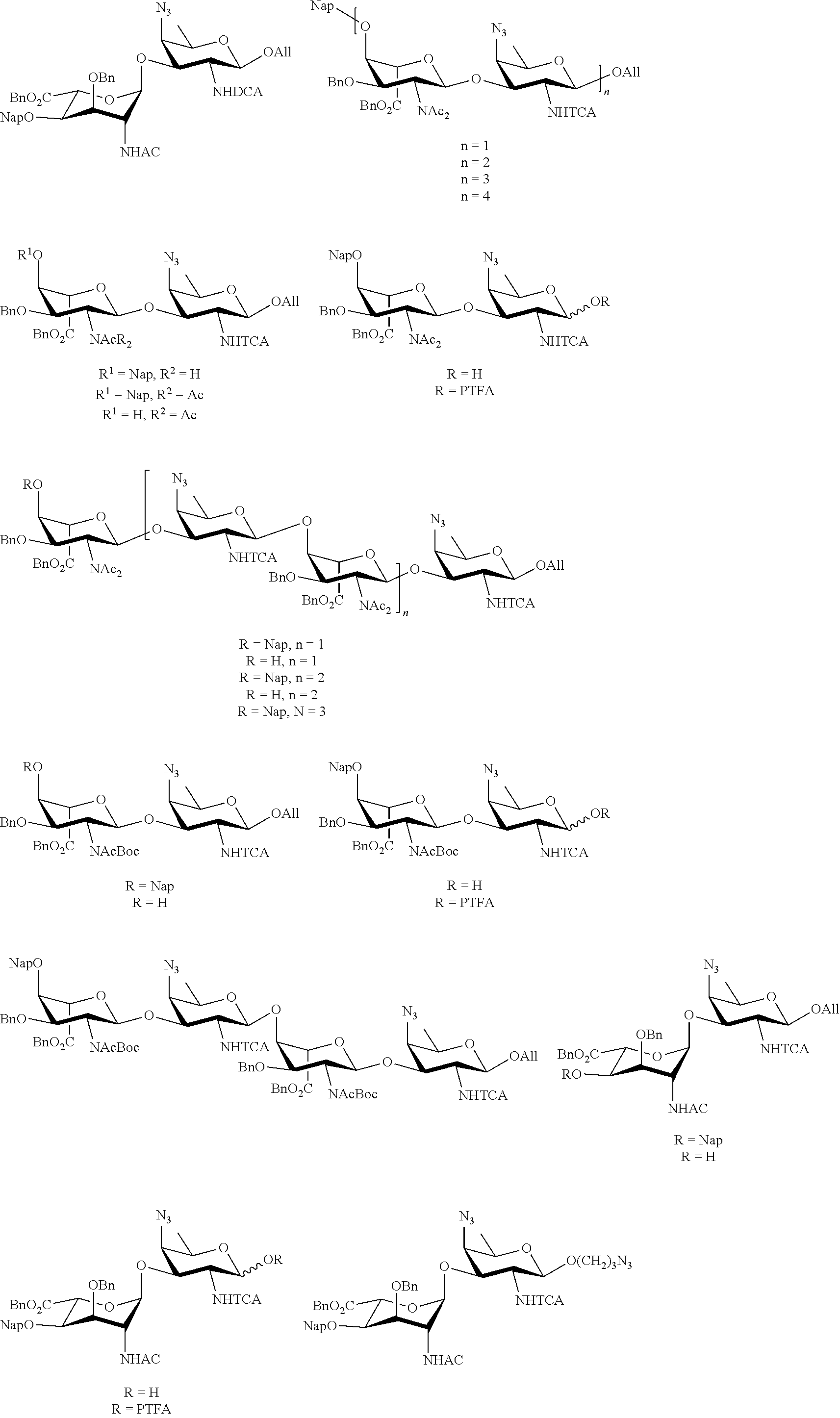

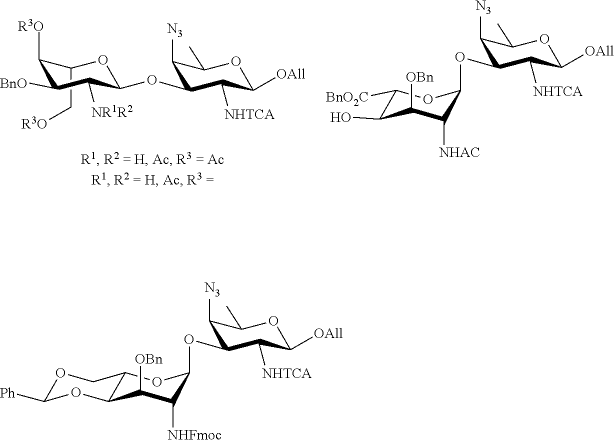

In another aspect, the invention concerns a compound of one of the following formulae:

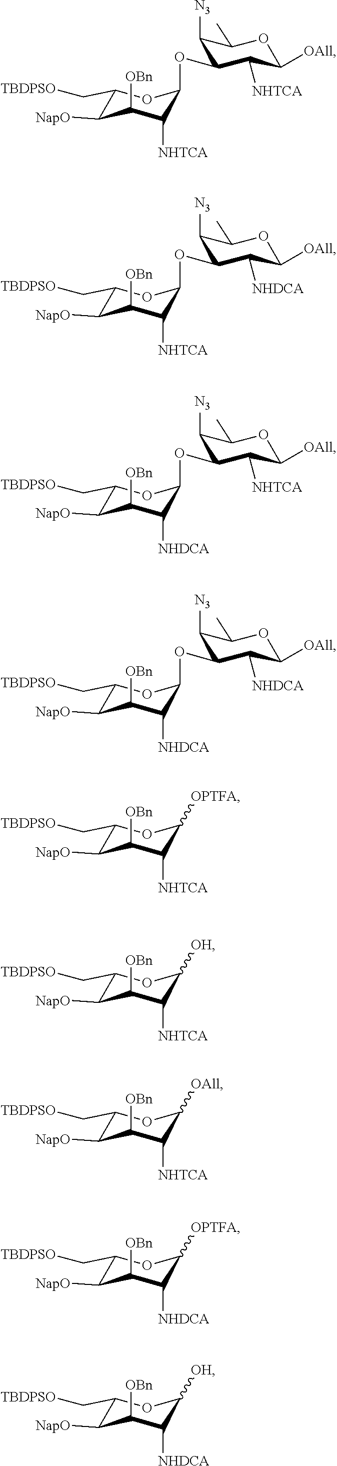

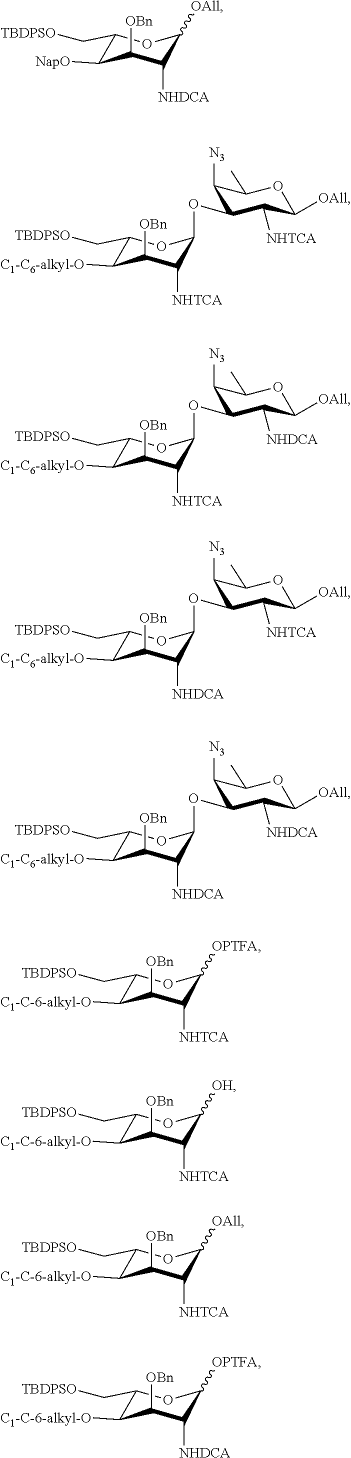

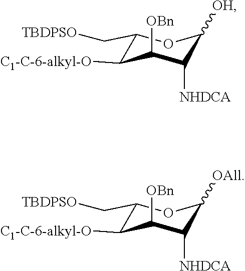

Protection and deprotection techniques (i.e. protecting group introduction and cleavage) are for instance described by P. G. M. Wuts and T. W. Greene (Greene's Protective Groups in Organic Synthesis, Fourth Edition; Wiley-Interscience, 2006; or Greene's Protective Groups in Organic Synthesis, fifth Edition; Wiley-Interscience, 2014, by P. Wuts, DOI: 10.1002/9781118905074). Reference is also made to “Recent Advances Toward Robust N-Protecting Groups for Glucosamine as Required for Glycosylation Strategies”, Mohamed Ramadan El Sayed Aly and El Sayed H. El Ashry, Advances in Carbohydrate Chemistry and Biochemistry, Volume 73, p. 117-224 (2016).

### Definitions

The following terms and expressions contained herein are defined as follows:

As used herein, the term “alkyl” refers to a straight-chain, or branched alkyl group having 1 to 6 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, etc. The alkyl moiety of alkyl-containing groups, such as aralkyl or O-alkyl groups, has the same meaning as alkyl defined above. Lower alkyl groups, which are preferred, are alkyl groups as defined above which contain 1 to 4 carbons. A designation such as “C₁-C₄ alkyl” refers to an alkyl radical containing from 1 to 4 carbon atoms.

As used herein, the term “aryl” refers to a substituted or unsubstituted, mono- or bicyclic hydrocarbon aromatic ring system having 6 to 10 ring carbon atoms. Examples include phenyl and naphthyl. Preferred aryl groups include unsubstituted or substituted phenyl and naphthyl groups. Included within the definition of “aryl” are fused ring systems, including, for example, ring systems in which an aromatic ring is fused to a cycloalkyl ring. Examples of such fused ring systems include, for example, indane, indene, and tetrahydronaphthalene.

As used herein, the term “heteroaryl” refers to an aromatic group containing 5 to 10 ring carbon atoms in which one or more ring carbon atoms are replaced by at least one hetero atom such as —O—, —N—, or —S—. Examples of heteroaryl groups include pyrrolyl, furanyl, thienyl, pirazolyl, imidazolyl, thiazolyl, isothiazolyl, isoxazolyl, oxazolyl, oxathiolyl, oxadiazolyl, triazolyl, oxatriazolyl, furazanyl, tetrazolyl, pyridyl, pyrazinyl, pyrimidinyl, pyridazinyl, triazinyl, indolyl, isoindolyl, indazolyl, benzofuranyl, isobenzofuranyl, purinyl, quinazolinyl, quinolyl, isoquinolyl, benzoimidazolyl, benzothiazolyl, benzothiophenyl, thianaphthenyl, benzoxazolyl, benzisoxazolyl, cinnolinyl, phthalazinyl, naphthyridinyl, and quinoxalinyl. Included within the definition of “heteroaryl” are fused ring systems, including, for example, ring systems in which an aromatic ring is fused to a heterocycloalkyl ring. Examples of such fused ring systems include, for example, phthalamide, phthalic anhydride, indoline, isoindoline, tetrahydroisoquinoline, chroman, isochroman, chromene, and isochromene.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

In another aspect, the present invention is directed to pharmaceutically acceptable salts of the compounds described above. As used herein, “pharmaceutically acceptable salts” includes salts of compounds of the present invention derived from the combination of such compounds with non-toxic acid.

Acid addition salts include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

In addition to pharmaceutically-acceptable salts, other salts are included in the invention. They may serve as intermediates in the purification of the compounds, in the preparation of other salts, or in the identification and characterization of the compounds or intermediates.

The pharmaceutically acceptable salts of compounds of the present invention can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present invention.

It is recognized that compounds of the present invention may exist in various stereoisomeric forms. As such, the compounds of the present invention include both diastereomers and enantiomers. The compounds are normally prepared as racemates and can conveniently be used as such, but individual enantiomers can be isolated or synthesized by conventional techniques if so desired. Such racemates and individual enantiomers and mixtures thereof form part of the present invention.

It is well known in the art how to prepare and isolate such optically active forms. Specific stereoisomers can be prepared by stereospecific synthesis using enantiomerically pure or enantiomerically enriched starting materials. The specific stereoisomers of either starting materials or products can be resolved and recovered by techniques known in the art, such as resolution of racemic forms, normal, reverse-phase, and chiral chromatography, recrystallization, enzymatic resolution, or fractional recrystallization of addition salts formed by reagents used for that purpose. Useful methods of resolving and recovering specific stereoisomers described in Eliel, E. L.; Wilen, S. H. Stereochemistry of Organic Compounds; Wiley: New York, 1994, and Jacques, J, et al. Enantiomers, Racemates, and Resolutions; Wiley: New York, 1981, each incorporated by reference herein in their entireties.

In particular, compounds of the invention differing in the value of n show conformational mimicry. For example, it has be shown by ¹H NMR studies that compounds of the invention with a n value of 2, 3, 4, 5 and 6 share a conformational mimicry.

As used herein, the term “oligosaccharide” more particularly refers to a saccharide containing from 2 to 10 monosaccharides (simple sugars).

As used herein, the term “polysaccharide” more particularly refers to a saccharide containing more than 10 monosaccharides (simple sugars).

As used herein, a range of values in the form “x-y” or “x to y”, or “x through y”, include integers x, y, and the integers there between. For example, the phrases “1-6”, or “1 to 6” or “1 through 6” are intended to include the integers 1, 2, 3, 4, 5, and 6. Preferred embodiments include each individual integer in the range, as well as any subcombination of integers. For example, preferred integers for “1-6” can include 1, 2, 3, 4, 5, 6, 1-2, 1-3, 1-4, 1-5, 2-3, 2-4, 2-5, 2-6, etc.

As used herein, the term “donor” more particularly refers to a mono-, oligo- or polysaccharide bearing a leaving group at the anomeric position.

As used herein, the term “acceptor” more particularly refers to a mono-, oligo- or polysaccharide having at least a free hydroxyl group, in general other than the anomeric hydroxyl, preferably at least the free hydroxyl group corresponding to the elongation site of the growing chain.

By “divalent C₁-C₁₂ alkyl, C₂-C₁₂ alkenyl or C₂-C₁₂ alkynyl chain” is in particular meant a C₁-C₁₂ alkane diyl, C₂-C₁₂ alkene diyl or C₂-C₁₂ alkyne diyl chain, respectively.

By “bound to the carrier via the reducing end of said oligo- or polysaccharide” is in particular meant, for example, the end indicated by an arrow as follows:

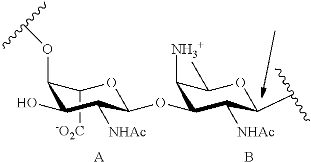

### FIGURES

FIG. 1 presents the anti-S. sonnei LPS IgG titer induced in mice receiving three injections of glycoconjugates SonB-SonF containing 2.5 μg of oligosaccharide per dose. Bleeding 3 weeks after the 3rd injection. X-axis: Glycoconjugates. Y-axis: Anti-S. sonnei LPS IgG titer. No statistically significant differences were observed between SonB, SonC and SonD. No statistically significant differences were observed between SonE and SonF. The Ab titers induced by these glycoconjugates were significantly higher than that induced by SonB, SonC and SonD. Medians are indicated (bold lines). T-test Mann Withney non parametric: *** p<0.0005.

FIG. 2 presents the anti-S. sonnei LPS IgG titer induced in mice receiving three injections of glycoconjugates Son F-Son M containing 2 μg of oligosaccharide per injection. Bleeding 1 month after the 3rd injection. X-axis: Glycoconjugates. Y-axis: Anti-S. sonnei LPS IgG titer. Medians are indicated (bold lines). T-test Mann Withney non parametric: *** p<0.0005; ** p<0.005; * p<0.05.

FIG. 3 presents the anti-S. sonnei LPS IgG titer induced in mice receiving three doses of glycoconjugates Son H, Son K and Son M (2 μg of oligosaccharide per injection). Bleeding were performed 30 days after immunization 1 (J30 imm1), 30 days after immunization 2 (J30 imm2) and 7 days and 30 days after immunization 3 (J7 imm3 and J30 imm3, respectively). X-axis: Glycoconjugates and timing of bleeding. Y-axis: Anti-S. sonnei LPS IgG titer. Medians are indicated (bold lines). T-test Mann Withney non parametric: *** p<0.0005; ** p<0.005. FIG. 4 presents the anti-S. sonnei LPS IgG titer induced in mice receiving three injections of conjugates Son W-Son Z containing 2 μg or 0.5 μg of oligosaccharide per injection. Bleeding was performed 3 weeks after the 3rd immunization. X-axis: Glycoconjugates and dose of oligosaccharide (2 for 2 μg and 0.5 for 0.5 μg, respectively). Y-axis: Anti-S. sonnei LPS TgG titer. Medians are indicated (bold lines).

FIG. 5 presents the anti-S. sonnei LPS IgG titer induced in mice receiving three injections of conjugates Son N-Son V containing 2 μg of oligosaccharide per injection. Bleeding was performed 3 weeks after the 3rd immunization. X-axis: Glycoconjugates. Y-axis: Anti-S. sonnei LPS IgG titer. Medians are indicated (bold lines). T-test Mann Withney non parametric: *** p<0.0005.

FIG. 6 presents the anti-S. sonnei LPS IgG titer induced in mice receiving two injections of conjugates Son N, Son AA and Son BA containing 2 μg of oligosaccharide per injection. Bleeding was performed 3 weeks after the 2nd immunization. X-axis: Glycoconjugates with alum (+AlH) or without. Y-axis: Anti-S. sonnei LPS IgG titer. Medians are indicated (bold lines). T-test Mann Withney non parametric: *** p<0.0005; ** p<0.005.

FIG. 7 presents the anti-S. sonnei LPS IgG subclasses induced in mice receiving three injections of conjugate Son Y. X-axis: mouse IgG subclasses. Y-axis: Anti-S. sonnei LPS IgG subclass titer. Medians are indicated (bold lines).

FIG. 8 presents the anti-S. sonnei LPS IgG titer induced in mice receiving two injections of conjugates Son CA-Son GA containing 2 μg or 1 μg of oligosaccharide per injection. Bleeding was performed 3 weeks after the 2nd immunization. X-axis: Glycoconjugates and dose of oligosaccharide (2 for 2 μg and 1 for 1 μg, respectively). Y-axis: Anti-S. sonnei LPS IgG titer. Medians are indicated (bold lines).

### EXAMPLES

General Procedures
Ref 1: [1] H. B. Pfister, L. A. Mulard, Org. Lett. 2014, 16, 4892-4895.
Ref 2: [2] Westphal, O., and J. Jann. 1965. Bacterial lipopolysaccharides extraction with phenol-water and further application of the procedures. Meth. Carbohydr. Chem. 5: 83-91.

Anhyd. solvents including Tol, DCM, DCE, THF, DMF, MeOH, ACN, and Py, were delivered on MS and used as received. Reactions requiring anhyd. conditions were run under an Ar atmosphere, using dried glassware. 4 Å MS were activated before use by heating under high vacuum. Analytical TLC was performed with silica gel 60 F254, 0.25 mm pre-coated TLC aluminium foil plates. Compounds were visualized using UV254 and/or orcinol (1 mg·mL⁻¹) in 10% aq. H₂SO₄ with charring. Flash column chromatography was carried out using silica gel (particle size 40-63 m). RP-HPLC purification was carried out using a Kromasil 5 μm C18 100 Å 10×250 mm semi-preparative column eluting with ACN in 0.08% aq. TFA, 5 mL·min⁻¹, with UV (λ=215 nm) detection. Analytical RP-HPLC of the final compounds (λ=215 nm or 230 nm, ESLD) was carried out using a Kromasil 3.5 μm C18 100 Å 3×150 mm analytical column, eluting with a 0-20% linear gradient of ACN in 0.08% aq. TFA over 20 min at a flow rate of 0.4 mL·min⁻¹ (conditions A) or 1.0 mL·min⁻¹ (conditions A′), a 0-20% linear gradient of ACN in 10 mM aq. ammonium acetate over 20 min at a flow rate of 0.4 mL·min⁻¹ (conditions B), or using a an Aeris Peptide 3.5 μm C18 100 Å 2.1×150 mm analytical column, eluting with a 0-20% linear gradient of ACN in 0.08% aq. TFA over 20 min at a flow rate of 0.3 mL·min⁻¹ (conditions C), or using a Kromasil 3.5 μm C18 100 Å 3×150 mm analytical column, eluting with a 0-40% linear gradient of ACN in 0.08% aq. TFA over 20 min at a flow rate of 0.4 mL·min⁻¹ (conditions D) or a 0-70% linear gradient of ACN in 0.08% aq. TFA over 20 min at a flow rate of 0.4 mL·min⁻¹ (conditions E). Except for octasaccharide 4, NMR spectra were recorded at 303 K on a Bruker Avance spectrometer equipped with a BBO probe at 400 MHz (¹H) and 100 MHz (¹³C). Spectra were recorded in CDCl₃, DMSO-d6 and D₂O. In the case of octasaccharide 4, NMR spectra were recorded on a 800 MHz Bruker Avance NEO equipped with a high sensitivity TCI cryogenic probe. Chemical shifts are reported in ppm (δ) relative to the residual solvent peak in the case of CDCl₃ and DMSO-d6, and to HOD and DSS (4,4-dimethyl-4-silapentane-1-sulfonic acid) in the case of D₂O, at 7.28/77.0, 2.50/39.0 and 4.70/0.00 ppm for the ¹H and ¹³C spectra, respectively. Coupling constants are reported in hertz (Hz). Elucidations of chemical structures were based on ¹H, COSY, DEPT-135, ¹³C, HSQC, HMBC, HSQCND and NOESY spectra. Signals are reported as s (singlet), d (doublet), t (triplet), dd (doublet of doublet), q (quadruplet), dt (doublet of triplet), dq (doublet of quartet), ddd (doublet of doublet of doublet), m (multiplet). Signals can also be described as broad (prefix br), or partially overlapped (suffix po). Of the two magnetically non-equivalent geminal protons at C-6, the one resonating at lower field is denoted H-6a, and the one at higher field is denoted H-6b. Sugar residues are lettered according to the lettering of the repeating unit of the S. sonnei O—Ag and identified by a subscript (A, B) in the listing of signal assignments. For compounds made of multiple repeating units, residues are distinguished in the form of A/B, Ai/Bi, with A/B corresponding to the repeating unit at the reducing end. HRMS spectra were recorded on a WATERS QTOF Micromass instrument in the positive-ion electrospray ionisation (ESI⁺) mode. Solutions were prepared using 1:1 ACN/H₂O containing 0.1% formic acid. In the case of sensitive compounds, solutions were prepared using 1:1 MeOH/H₂O to which was added 10 mM ammonium acetate.

General Procedure for Anomeric Deallylation

[Ir(COD)(PMePh₂)₂]PF₆ (0.02 equiv.) was dissolved in anhyd. THF (20 mM) and stirred for 30-40 min under an H₂ atmosphere. The resulting yellow solution was degassed several times with Ar and poured into a solution of allyl glycoside (1.0 equiv.) in anhyd. THF (50-100 mM). After stirring at rt for 1-2 h, NIS (1.1 equiv.) and H₂O, to reach a 1:5 H₂O/THF ratio, were added. After stirring at rt for 1 h, the reaction was quenched by addition of 10% aq. sodium sulphite. The reaction mixture was concentrated and the aq. phase was extracted with DCM. The combined organic layer was washed with brine, dried over anhyd. Na₂SO₄ and concentrated. Purification by flash chromatography (cHex/EtOAc) yielded the expected hemiacetal as a α/β mixture.

General Procedure for the Synthesis of the PTFA Glycosyl Donors

The hemiacetal precursor (1.0 equiv.) was dissolved in acetone (0.2 M). PTFACl (1.3 equiv.) was added followed by addition of Cs₂CO₃ (1.1 equiv.). After stirring at rt under an Ar atmosphere until completion (estimated ˜2 h), the reaction mixture was filtered over a plug of Celite and washed exhaustively with anhydr. DCM. The filtrate was concentrated under reduced pressure. The crude residue was used as such in the glycosylation reaction. Purification by flash chromatography (cHex/EtOAc containing 1% Et₃N) provided analytical samples.

General procedure for hydrogenation enabling the concomitant cleavage of the benzyl esters and benzyl ethers, reduction of azides to amines and allyl aglycon to propyl aglycon, and hydrodechlorination of the trichloroacetamides into acetamides.

Protocol 1: The oligosaccharide (50 mg) was dissolved in 2-MeTHF/isopropanol/water (1:15:3, v/v/v). 20% Pd(OH)₂/C (100 mg, twice the mass of oligosaccharide) was added and the reaction mixture was degassed several times and vigorously stirred under a hydrogen atmosphere for 24 h. After each 1 h, the pH of the solution was checked and the solution was neutralized by addition of 1M aq. NaHCO₃ (3 equiv. per NHTCA group added within the first 6-12 h). Reaction progress was monitored by LC-MS and HRMS. In average, completion was reached within 12-24 h. The suspension was filtered by passing through a 0.2 μm filter, and solids were washed repeatedly with water. Volatiles were removed under vacuum, water was added and the solution was lyophilized. The crude product was purified by preparative RP-HPLC. Fractions of interest were pooled and lyophilized to give the fully deprotected oligosaccharide as confirmed by HRMS and NMR analysis.

Protocol 2 (H-cube, full hydrogen mode, Pressure 0 bar, column heater: 25° C., flow rate: 0.8-1.2 mL·min⁻¹, 20% Pd(OH)₂—C cartridge): 50 mg of oligosaccharide were dissolved in 2-MeTHF/isopropanol/water (1:15:3, v/v/v). The solution was subjected to hydrogenation. After each cycle, the released HCl released was quenched by addition of 1 M aq. NaHCO₃ (3 equiv. per NHTCA group added within first 3-6 cycles). Reaction progress was monitored by LC-MS and HRMS analysis. In average, completion was reached after 6-12 cycles. The suspension was filtered by passing through a 0.2 μm filter and solids were washed repeatedly with water. Volatiles were removed under vacuum, water was added, and the solution was lyophilized. The crude product was purified by preparative RP-HPLC. Fractions of interest were pooled and lyophilized to give the fully deprotected oligosaccharide as confirmed by HRMS and NMR analysis.

Protocol 3 (10% Pd—C): 50 mg of protected oligosaccharide were dissolved in 1.0 mL 2-MeTHF and 2-MeTHF/isopropanol/water (1:10:1, v/v/v) was added to reach 0.2-0.25 mM/repeating unit. The reaction mixture was degassed several times, 10% Pd/C (twice the mass of the starting oligosaccharide) was added and the suspension was stirred vigorously under a hydrogen atmosphere (balloon) until RP-HPLC and HRMS monitoring indicated reaction completion. More 10% Pd/C was added over time if needed (up to twice the mass of the starting oligosaccharide). The pH of the solution was checked regularly and adjusted to 5-6 by addition of 1 M aq. NaHCO₃ (up to 3 equiv. per NHTCA group). The suspension was filtered by passing through a pad of Celite and solids were washed repeatedly with water. Volatiles were removed under vacuum, water was added, and the solution was lyophilized. The crude product was purified by preparative RP-HPLC. Fractions of interest were pooled and lyophilized to give the fully deprotected oligosaccharide as confirmed by HRMS and NMR analysis.

Useful Intermediates

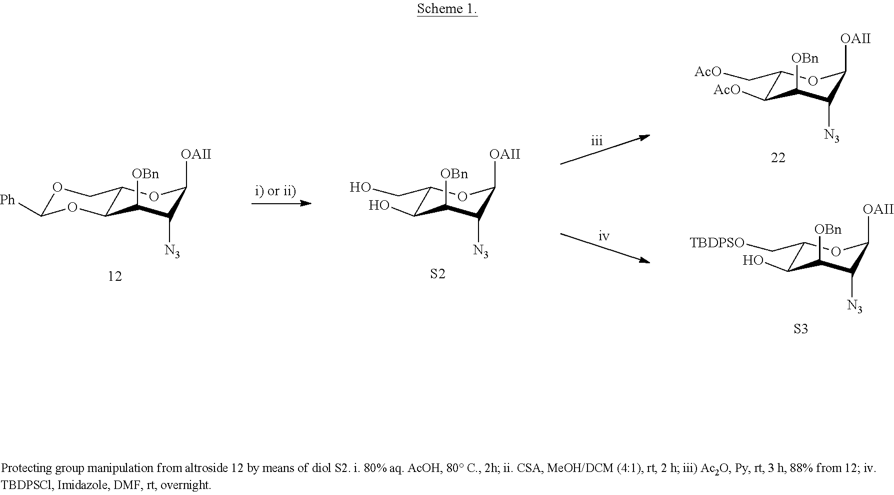

Allyl 2-azido-3-O-benzyl-2-deoxy-α-L-altropyranoside (S2). Azide 12 (4.0 g, 9.4 mmol, 1.0 equiv.) was dissolved in AcOH (40 mL) and water (10 mL) was added. The solution was heated at 80° C. for 2 h. A TLC follow up (Tol/EtOAc 1:1) showed the absence of the starting material (R_{f} 0.9) and the presence of a new spot (R_{f} 0.1). After reaching rt, the reaction mixture was concentrated under vacuum. The residue was coevaporated with toluene (10 mL) twice and dried over vacuum to deliver the crude diol S2. The latter had R_{f} 0.45 (Tol/EtOAc 1:1). ¹H NMR (CDCl₃) δ 7.38-7.34 (m, 5H, H_{Ar}), 5.99-5.90 (m, 1H, CH_{All}), 5.36-5.31 (m, 1H, CH_{2All}), 5.25-5.22 (m, 1H, CH_{2All}), 4.83 (dₚₒ, 1H, J=11.2 Hz, CH_{2Bn}), 4.80 (dₚₒ, 1H, J_{1,2}=2.4 Hz, H-1), 4.61 (d, 1H, CH_{2Bn}), 4.29-4.23 (m, 1H, CH_{2All}), 4.07-4.01 (m, 1H, CH_{2All}), 3.99 (ddd, 1H, J_{4,5}=8.4 Hz, H-5), 3.92-3.88 (m, 1H, H-4) 3.89 (ddₚₒ, 1H, J_{2,3}=3.5 Hz, H-2), 3.86 (ddₚₒ, 1H, J_{5,6b}=3.6 Hz, H-6a), 3.79 (ddₚₒ, 1H, J_{5,6a}=5.2 Hz, J_{6a,6b}=12.3 Hz, H-6b), 3.78-3.75 (mₒ, 1H, H-3). ¹³C NMR (CDCl₃) δ 137.0 (C_{q, Ar}), 133.6 (CH_{All}), 129.0, 128.6, 128.2, 128.2, 128.0 (C_{Ar}), 117.4 (CH_{2All}), 97.9 (C-1_{A}, ¹J_{C,H}=170 Hz), 76.2 (C-3), 72.5 (CH_{2Bn}), 70.8 (C-5), 68.7 (CH_{2All}), 64.7 (C-4), 62.9 (C-6), 59.6 (C-2). HRMS (ESI⁺): m/z [M+NH₄]⁺ calcd for C₁₆H₂₅N₄O₅, 353.1825; found 353.1827.

Allyl 4,6-di-O-acetyl-2-azido-3-O-benzyl-2-deoxy-α-L-altropyranoside (22). The crude diol S2 (9.4 mmol theo., 1.0 equiv.) was dissolved in pyridine (40 mL) and acetic anhydride (2.2 mL, 23.6 mmol, 2.5 equiv.) was added at rt. After stirring for 3 h, a TLC analysis indicated reaction completion. Volatiles were evaporated and coevaporated with toluene (10 mL) twice, diluted with DCM (150 mL). The organic phase was washed with 1N aq. HCl (150 mL), 50% aq. NaHCO₃ (150 mL) and brine (100 mL), dried over Na₂SO₄, filtered, and concentrated under vacuum. The crude was purified by flash chromatography eluting with Tol/EtOAc (4:1→3:1) to give diacetate 22 (3.5 g, 8.3 mmol, 88%) as a white solid. Azide 22 had R_{f} 0.4 (Tol/EtOAc 4:1). ¹H NMR (CDCl₃) δ 7.39-7.31 (m, 5H, H_{Ar}), 5.98-5.89 (m, 1H, CH_{All}), 5.37-5.31 (m, 1H, CH_{2All}), 5.26-5.22 (m, 2H, H-4, CH_{2All}), 4.72 (d, 1H, J_{1,2}=4.4 Hz, H-1), 4.67 (ddₚₒ, 1H, J=11.6 Hz, CH_{2Bn}), 4.29 (ddₚₒ, 1H, J_{5,6a}=6.0 Hz, H-6a), 4.29-4.24 (m, 2H, H-5, CH_{2All}), 4.18-4.15 (mₚₒ, 1H, H-6b), 4.09-4.04 (m, 1H, CH_{2All}), 3.81 (dd, 1H, J_{2,3}=8.3 Hz, H-2), 3.81 (dd, 1H, J_{3,4}=3.8 Hz, H-3), 2.10 (s, 3H, CH_{3Ac}), 2.08 (s, 3H, CH_{3Ac}). ¹³C NMR (CDCl₃) δ 170.4, 170.0 (CO_{Ac}), 137.1 (C_{q,Ar}), 133.4 (CH_{All}), 128.4, 128.0 (C_{Ar}), 117.3 (CH_{2All}), 98.7 (C-1_{A}, ¹J_{C,H}=169 Hz), 74.6 (C-3), 72.4 (CH_{2Bn}), 69.6 (C-5), 69.2 (CH_{2All}), 66.3 (C-4), 62.7 (C-6), 61.5 (C-2), 20.8 (CH_{3Ac}), 20.6 (CH_{3Ac}). HRMS (ESI⁺): m/z [M+Na]⁺ calcd for C₂₀H₂₅N₃O₇Na, 442.1590; found 442.1597.

Allyl 2-azido-3-O-benzyl-6-O-tert-butyldiphenylsilyl-2-deoxy-α-L-altropyranoside (S3). CSA (4.1 g, 17.7 mmol, 0.5 equiv.) was added to acetal 12 (15.0 g, 35.4 mmol, 1.0 equiv.) in MeOH/DCM (4:1, 170 mL). After stirring at rt for 2 h, a TLC follow up (Tol/EtOAc 4:1) indicated reaction completion. 5% Aq. NaHCO₃ (300 mL) was added followed by EtOAc (500 mL). The organic phase was separated, washed with brine (500 mL), dried over Na₂SO₄ and concentrated under reduced pressure. The material was dried under high vacuum to give the crude diol S2 as a yellow oil. The latter was used as such in the next step.

tert-Butyldiphenylchlorosilane (10.1 mL, 38.9 mmol, 1.1 equiv.) and imidazole (3.1 g, 46.0 mmol, 1.3 equiv.) were added to diol S2 in anhyd. DMF (180 mL) at 0° C. The reaction mixture was allowed to reach rt slowly and stirred overnight at this temperature. MeOH (10.0 mL) was added and after 30 min, volatiles were evaporated under reduced pressure. The crude material was dissolved in EtOAc (500 mL) and the organic layer was washed with 90% aq. brine (500 mL), separated, dried over Na₂SO₄, and concentrated to give the crude silyl ether S3. The latter had R_{f} 0.65 (Tol/EtOAc 9:1). ¹H NMR (CDCl₃) δ 7.71-7.68 (m, 4H, H_{Ar}), 7.40-7.34 (m, 11H, H_{Ar}), 6.00-5.90 (m, 1H, CH_{All}), 5.35-5.29 (m, 1H, CH_{2All}), 5.23-5.19 (m, 1H, CH_{2All}), 4.84 (d, 1H, J_{1,2}=4.2 Hz, H-1), 4.80 (d, 1H, J=11.4 Hz, CH_{2Bn}), 4.62 (d, 1H, CH_{2Bn}), 4.34-4.29 (m, 1H, CH_{2All}), 4.09-4.04 (m, 1H, CH_{2All}), 4.01 (ddd, 1H, H-5), 3.95 (ddd, 1H, J_{4,5}=6.7 Hz, H-4), 3.91 (dd, 1H, J_{5,6b}=3.3 Hz, J_{6a,6b}=11.2 Hz, H-6a), 3.85-3.80 (m, 2H, J_{5,6b}=5.2 Hz, H-6b, H-2), 3.77 (dd, 1H, J_{2,3}=7.3 Hz, J_{3,4}=3.9 Hz, H-3), 3.77 (dd, 1H, J_{4,OH}=5.2 Hz, OH-4). ¹³C NMR (CDCl₃) δ 137.2, 135.7, 135.6 (C_{q,Ar}), 133.8 (CH_{All}), 133.2, 133.1, 129.7 (2C), 128.6, 128.1, 128.0, 127.7 (C_{Ar}), 117.3 (CH_{2All}), 98.2 (C-1_{A}, ¹J_{C,H}=169 Hz), 76.9 (C-3), 73.4 (C-5), 72.6 (CH_{2Bn}), 68.6 (CH_{2All}), 65.2 (C-4), 64.5 (C-6), 60.8 (C-2), 26.8 (CH_{3,TBDPS}), 19.2 (C_{TBDPS}). HRMS (ESI⁺): m/z [M+NH₄]⁺ calcd for C₃₂H₄₃N₄O₅Si, 591.3003; found 591.2971.

### Example 1: Strategy 2_{A}-NHAc,2_{B}-NTCA, 4_{A}-Nap

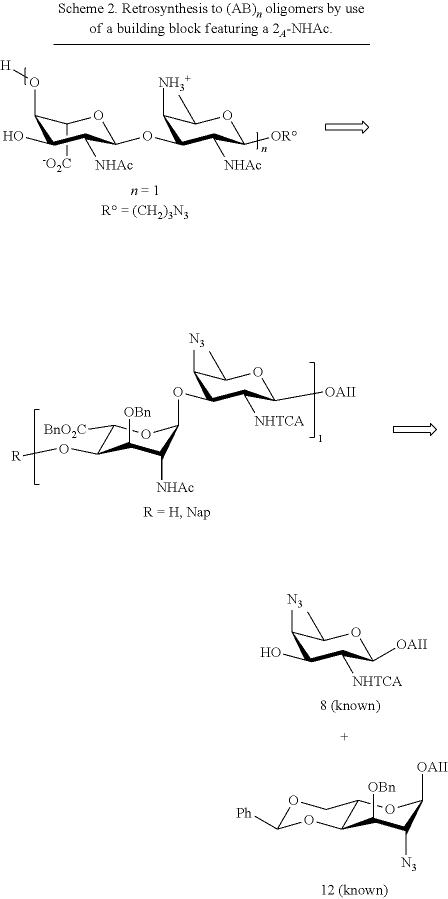

Disaccharide Building Block
AB acceptor: from a 4_{A},6_{A}-O-benzylidene A donor

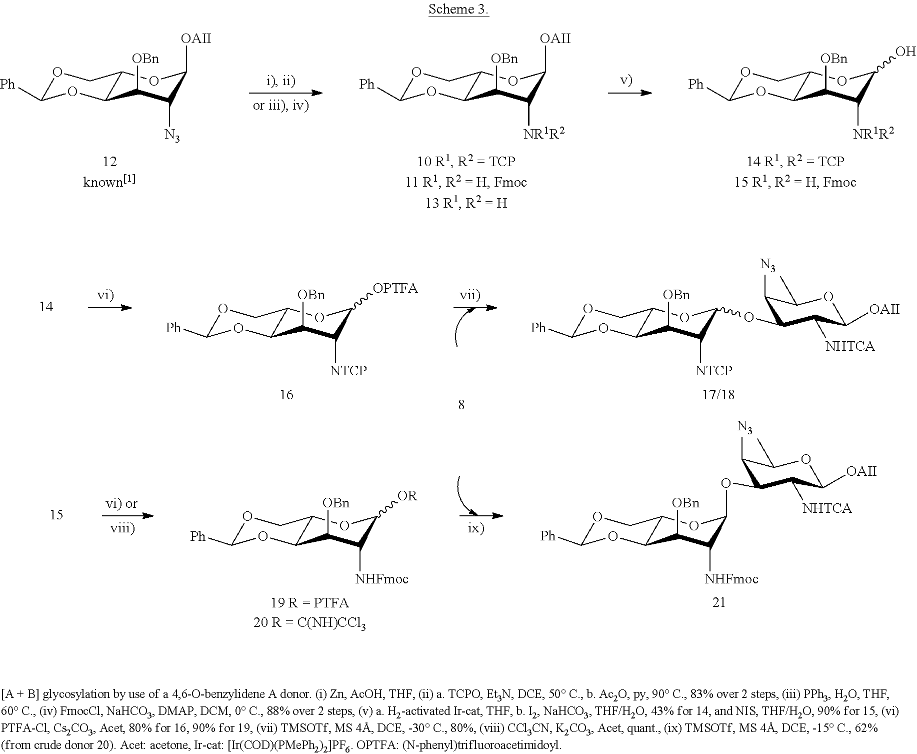

Allyl 3-O-benzyl-4,6-O-benzylidene-2-deoxy-2-tetrachlorophthalimido-α-L-altropyranoside (10). The known azide 12^{[1]} (10.0 g, 23.6 mmol, 1.0 equiv.) was dissolved in anhyd. THF (95 mL). Zn dust (12.3 g, 189 mmol, 8.0 equiv.) and AcOH (10.8 mL, 189 mmol, 8.0 equiv.) were added at rt. The reaction mixture was stirred vigorously for 1 h. A TLC follow up (Tol/EtOAc 1:1) showed the formation of a highly polar product and the full consumption of the starting 12 (R_{f} 0.9). The suspension was filtered through a pad of Celite and washed with DCM (100 mL) twice. The filtrate was washed with satd. aq. NaHCO₃. The organic layer was dried over anhyd. Na₂SO₄, filtered, and concentrated under reduced pressure. The residue, corresponding to the known amine 13^{[1]} was dried under high vacuum for 2 h. The crude material (9.6 g) was subjected to the next step.

Tetrachlorophthalic anhydride (4.05 g, 14.1 mmol, 0.6 equiv.) was added to the crude 13 (9.38 g, 23.6 mmol theo.) stirred in anhyd. DCM (100 mL) at rt under an Ar atmosphere. After 30 min, Et₃N (3.2 mL, 23.6 mmol, 1.0 equiv.) followed by more TCPO (4.05 g, 14.1 mmol, 0.6 equiv.) were added. The reaction mixture was stirred for another 30 min at rt, at which time a TLC follow up (EtOAc) revealed the presence of a polar product (R_{f} 0.0) and absence of 13 (R_{f} 0.15). Volatiles were eliminated under reduced pressure and the residue was dried under high vacuum for 1 h. The crude was dissolved in anhyd. Py (90 mL) and Ac₂O (11.1 mL, 118 mmol, 5.0 equiv.) was added at rt. The mixture was heated to 80° C. for 10 min, at which time a TLC follow up indicated completion. Volatiles were eliminated under reduced pressure and coevaporated with toluene (40 mL) twice. The crude was diluted with DCM (200 mL) and washed with 1N aq. HCl (300 mL), satd. aq. NaHCO₃ (300 mL) and brine (250 mL). The DCM layer was dried over Na₂SO₄, filtered and concentrated. The crude was purified by flash chromatography (cHex/EtOAc, 98:2 to 90:10) to give the fully protected 10 (13.0 g, 18.8 mmol, 83%) as a dense yellowish oil. Allyl glycoside 10 had R_{f} 0.65 (Tol/EtOAc 10:1). ¹H NMR (CDCl₃) δ 7.53-7.05 (m, 10H, H_{Ar}), 5.88-5.80 (m, 1H, CH_{All}), 5.65 (s, 1H, H_{Bzl}), 5.28-5.23 (m, 1H, CH_{2All}), 5.16-5.12 (mₚₒ, 1H, CH_{2All}), 5.14 (dₚₒ, 1H, J_{1,2}=4.0 Hz, H-1), 4.85 (d, 1H, J=12.5 Hz, CH_{2Bn}), 3.92 (t, 1H, J_{2,3}=4.0 Hz, H-2), 4.71 (d, 1H, CH_{2Bn}), 4.52-4.42 (m, 2H, H-5, H-6a), 4.34 (ddₚₒ, 1H, J_{3,4}=4.4 Hz, J_{4,5}=9.6 Hz, H-4), 4.27-4.22 (m, 1H, CH_{2All}), 4.10 (t, 1H, H-3), 4.03-3.97 (m, 1H, CH_{2All}), 3.88 (t, 1H, J_{5,6b}=J_{6a,6b}=10.0 Hz, H-6b). ¹³C NMR (CDCl₃), δ 162.3 (CO_{NTCP}), 140.3, 138.1, 137.6, 137.6 (C_{q, Ar}), 133.6 (CH_{All}), 129.8, 129.0, 128.2, 128.0 (2C), 127.2, 126.9, 126.4 (C_{Ar}), 117.3 (CH_{2All}), 101.8 (C_{Bzl}), 95.4 (C-1_{A}, ¹J_{C,H}=171 Hz), 76.4 (C-4), 73.1 (C-3), 72.8 (CH_{2Bn}), 69.7 (C-6), 68.4 (CH_{2All}), 59.8 (C-5), 55.5 (C-2). HRMS (ESI⁺): m/z [M+Na]⁺ calcd for C₃₁H₂₅Cl₄NO₇Na, 686.0283; found 686.0284.

Allyl 3-O-benzyl-4,6-O-benzylidene-2-deoxy-2-N-(9-fluorenylmethoxycarbonyl)-α-L-altropyranoside (11). A solution of azide 12 (2.4 g, 5.6 mmol, 1.0 equiv.) in THF (30 mL) was added triphenylphosphine (1.63 g, 6.2 mmol, 1.1 equiv.) and H₂O (3.0 mL, 169 mmol, 30 equiv.). The reaction mixture was stirred overnight at 60° C., cooled to rt, concentrated under reduced pressure and coevaporated with toluene (10 mL) twice. The crude amine 13 in anhyd. DCM (28 mL) was stirred over freshly activated MS 4 Å for 30 min at rt under an Ar atmosphere. After cooling to 0° C., NaHCO₃ (953 mg, 11.3 mmol, 2.0 equiv.), DMAP (69 mg, 567 μmol, 0.1 equiv.) and FmocCl (1.7 g, 6.8 mmol, 1.2 equiv.) were added. After stirring for 1 h at this temperature, a TLC analysis (cHex/EtOAc 9:1) revealed the conversion of intermediate 13 (R_{f} 0.45) into a less polar product (R_{f} 0.7). The reaction mixture was filtered and solids were washed with DCM (20 mL) twice. The filtrate was concentrated in vacuo and the residue was purified by flash chromatography using cHex/EtOAc (20:1-15:1) to give the Fmoc derivative 11 (3.1 g, 5.0 mmol, 88%) as a white solid. The fully protected 11 had ¹H NMR (CDCl₃) δ 7.81-7.78 (m, 2H, H_{Ar}), 7.65-7.60 (m, 2H, H_{Ar}), 7.51-7.26 (m, 15H, H_{Ar}), 5.98-5.89 (m, 1H, CH_{All}), 5.57 (s, 1H, H_{Bzl}), 5.37-5.31 (m, 1H, CH_{2All}), 5.22-5.20 (m, 1H, CH_{2All}), 4.96 (d, J_{2,NH}=8.8 Hz, NH), 4.88 (d, 1H, J=12.6 Hz, CH_{2Bn}), 4.83 (d, 1H, CH_{2Bn}), 4.73 (s, 1H, H-1), 4.59-4.48 (m, 3H, H-5, CH_{2Fmoc}), 4.32 (dd, 1H, J_{5,6a}=5.6 Hz, J_{6a,6b}=10.4 Hz, H-6a), 4.29-4.21 (m, 3H, CH_{Fmoc}, H-2, CH_{2All}), 4.04-3.99 (m, 1H, CH_{2All}), 3.90 (brs, 1H, H-3), 3.73 (tₚₒ, 1H, J_{5,6b}=10.4 Hz, H-6b), 3.70 (brdₚₒ, 1H, J_{4,5}=8.8 Hz, H-4). ¹³C NMR (CDCl₃) δ 155.2 (CO_{NHFmoc}), 144.3 143.6, 141.5, 141.4, 138.7, 137.6 (C_{q,Ar}), 133.6 (CH_{All}), 129.0, 128.2, 128.0, 127.8, 127.5, 127.3, 127.1, 127.0, 126.2, 124.7, 120.0 (C_{Ar}), 117.2 (CH_{2All}), 102.3 (C_{Bzl}), 98.9 (C-1_{A}, ¹J_{C,H}=170 Hz), 77.0 (C-4), 74.2 (C-3), 72.1 (CH_{2Bn}), 69.2 (C-6), 68.4, (CH_{2All}), 66.6 (CH_{2Fmoc}), 58.7 (C-5), 52.1 (C-2), 47.3 (CH_{Fmoc}). HRMS (ESI⁺): m/z [M+Na]⁺ calcd for C₃₈H₃₇NO₇Na, 642.2468; found 642.2464.

3-O-Benzyl-4,6-O-benzylidene-2-deoxy-2-tetrachlorophthalimido-α/β-L-altropyranose (14). [Ir(COD)(PMePh₂)₂]⁺PF₆⁻ (38 mg, 45 μmol, 0.02 equiv.) was dissolved in anhyd. THF (5.0 mL) and the red solution was stirred for 30 min under an H₂ atmosphere. The resulting light yellow solution was degassed several times with Ar and poured into a solution of allyl glycoside 10 (1.5 g, 2.26 mmol, 1.0 equiv.) in anhyd. THF (25 mL). After stirring at rt for 2 h, a TLC follow up (cHex/EtOAc; 15:1) showed the absence of the starting 10 (R_{f} 0.3) and the presence of a less polar spot (R_{f} 0.35). Iodine (1.14 g, 6.1 mmol, 1.05 equiv.) immediately followed by NaHCO₃ (571 mg, 6.78 mmol, 3.0 eqv.) in water (5 mL) were added. After stirring at rt for 1 h, the reaction was quenched by addition of 10% aq. Na₂SO₃. Volatiles were evaporated and the aq. layer was extracted with DCM (100 mL) twice. Purification by flash chromatography using cHex/EtOAc (10:1→8:1) gave the expected hemiacetal 14 (610 mg, 0.98 mmol, 43%) as a white floppy solid corresponding mainly to a single anomer. The later had R_{f} 0.15 (Tol/EtOAc; 10:1). ¹H NMR (CDCl₃) δ 7.55-7.52 (m, 2H, H_{Ar}), 7.44-7.38 (m, 3H, H_{Ar}), 7.11-7.09 (m, 2H, H_{Ar}), 6.94 (t, 2H, J=7.6 Hz, H_{Ar}), 6.78-6.74 (m, 1H, H_{Ar}), 6.35 (dd, 1H, J_{1,OH}=8.8 Hz, J_{1,2}=4.8 Hz, H-1), 5.61 (s, 1H, H_{Bzl}), 4.84 (d, 1H, J=12.4 Hz, CH_{2Bn}), 4.46 (dd, 1H, J_{5,6a}=5.2 Hz, J_{6a,6b}=10.4 Hz, H-6a), 4.39 (d, 1H, CH_{2Bn}), 4.30-4.24 (m, 2H, H-3, H-5), 4.02 (dd, 1H, J_{2,3}=3.2 Hz, H-2), 3.87-3.81 (m, 3H, H-6b, H-4, OH). ¹³C NMR (CDCl₃), δ 162.9 (CO_{NTCP}), 139.6, 137.7, 137.3 (C_{q,Ar}), 129.4, 129.1, 128.9, 128.3, 127.9, 127.0 (2C), 126.1 (C_{Ar}), 102.0 (C_{Bzl}), 91.1 (C-1_{A}, ¹J_{C,H}=176 Hz), 79.3 (C-4), 74.0 (CH_{2Bn}), 73.9 (C-3), 69.0 (C-6), 64.4 (C-5), 58.4 (C-2). HRMS (ESI⁺): m/z [M+Na]⁺ calcd for C₂₈H₂₁Cl₄NO₇Na, 645.9970; found 645.9964.

3-O-Benzyl-4,6-O-benzylidene-2-deoxy-2-N-(9-fluorenylmethoxycarbonyl)-α/β-L-altropyranose (15). [Ir(COD)(PMePh₂)₂]⁺PF₆⁻ (71 mg, 84 μmol, 0.02 equiv.) in anhyd. THF (5.0 mL) was stirred under an H₂ atmosphere for 1 h at rt. The resulting yellow solution was degassed several times with Ar and transferred by use of a cannula into a solution of allyl glycoside 11 (2.6 g, 4.1 mmol, 1.0 equiv.) in anhyd. THF (40 mL). After stirring at rt for 1 h, NIS (1.03 g, 4.6 mmol, 1.1 equiv.) and H₂O (8 mL) were added and the reaction mixture was stirred for an additional hour. At completion, the reaction was quenched by addition of 10% aq. Na₂SO₃, and volatiles were eliminated. The aq. phase was extracted with DCM (40 mL) twice. The combined organic phases were dried over Na₂SO₄, filtered and concentrated under reduced pressure. Purification of the residue by flash chromatography using Tol/EtOAc (5:1→4:1) yielded the expected 15 (2.2 g, 3.7 mmol, 90%) as a white solid. Hemiacetal 15 was isolated as a 7:3 α/β mix and had R_{f} 0.2 (Tol/EtOAc, 4:1). ¹H NMR (Partial assignment, CDCl₃) δ 7.79-7.77 (m, 2.7H, H_{Ar}), 7.76-7.62 (m, 2.6H, NH, H_{Ar}), 7.54-7.51 (m, 2.7H, H_{Ar}), 7.43-7.28 (m, 19.5H, H_{Ar}), 7.22-7.18 (m, 5.0H, H_{Ar}), 5.60 (s, 0.4H, H_{Bzl,β}), 5.57 (s, 1H, H_{Bzl,α}), 5.35-5.30 (m, 1H, H-1_{α}), 5.01-4.90 (m, 1.9H, H-1β, CH_{2Bnα}, CH_{2Bnβ}), 4.78-4.75 (m, 1.4H, CH_{2Bnα}, CH_{2Bnβ}), 4.58-4.55 (m, 2.6H, CH_{2Fmoc,αβ}), 4.34-4.30 (m, 1H, H-6a_{α}), 4.26-4.13 (m, 5H, H-2_{α}, H-2_{β}, H-3_{α}, H-3_{β}, H-5_{α}, H-5_{β}, CH_{Fmoc,α}, CH_{Fmoc,β}), 3.83-3.69 (m, 2.9H, H-4_{α}, H-4_{β}, H-6b_{β}), 3.62 (brs, 0.9H, OH_{αβ}). ¹³C NMR (CDCl₃), δ 156.4 (CO_{Fmoc}), 143.6, 143.5, 141.4, 141.3, 138.3, 137.8, 137.4, 137.0 (C_{q,Ar}), 129.2, 129.1, 129.0, 128.5, 128.3 (2C), 128.2, 128.0, 127.8, 127.6, 127.1, 126.1 (2C), 125.3, 124.9, 124.8, 124.7, 120.0 (C_{Ar}), 102.6 (C_{Bzl}), 102.3 (C_{Bzl}), 94.7 (C-1_{β}, ¹J_{C,H}=175.6 Hz), 92.0 (C-1_{A}, ¹J_{C,H}=170.6 Hz), 77.3 (C-4_{α}, C-4_{β}), 75.3 (C-3_{α}), 75.3 (C-3_{β}), 74.5 (CH_{2Bn, β}), 73.5 (CH_{2Bn, α}), 69.3 (C-6_{α}), 68.9 (C-6_{β}), 66.8 (CH_{2Fmoc,β}), 66.8 (CH_{2Fmoc,α}), 64.5 (C-5_{α}, C-5_{β}), 54.6 (C-2_{α}), 52.7 (C-2_{β}), 47.3 (CH_{Fmoc,α}, CH_{Fmoc,β}). HRMS (ESI⁺): m/z [M+Na]⁺ calcd for C₃₅H₃₃NO₇Na, 602.2155; found 602.2153.

3-O-Benzyl-4,6-O-benzylidene-2-deoxy-2-tetrachlorophthalimido-α/β-L-altropyranosyl (N-phenyl)trifluoroacetimidate (16). Hemiacetal 14 (2.85 g, 4.2 mmol, 1.0 equiv.) was dissolved in acetone (40 mL) and PTFACl (855 μL, 5.5 mmol, 1.3 equiv.) was added followed by addition of Cs₂CO₃ (1.68 g, 5.1 mmol, 1.1 equiv.). After stirring for 2 h at rt, a TLC follow (Tol/EtOAc 10:1) showed the presence of a less polar spot (R_{f} 0.7) and only traces of hemiacetal 14. The reaction mixture was filtered over a pad of Celite and washed with acetone (10 mL) twice. The filtrate was concentrated under reduced pressure and the residue was purified by flash chromatography (cHex/EtOAc 95:5→93:7 containing 1% Et₃N) to give donor 16 as a white solid (2.75 g, 3.46 mmol, 80%). The latter, obtained as a 10:3 mixture of two anomers, had R_{f} 0.3 (cHex/EtOAc 10:1). ¹H NMR (partial assignment, CDCl₃) δ 7.76 (brs, 0.8H, H_{Ar}), 7.59 (d, 0.8H, J=7.6 Hz, HM), 7.52-7.08 (m, 18.4H, H_{Ar}), 6.98 (d, 0.4H, J=7.2 Hz, HM), 6.82 (d, 1.6H, J=7.2 Hz, H_{Ar}), 5.50 (s, 0.8H, CH_{Bzl}), 5.24 (s, 0.2H, CH_{Bzl}), 5.01-4.96 (m, 1.8H), 4.76-4.66 (m, 1.3H), 4.35 (dd, 0.8H, J=5.2 Hz, J=10.8 Hz), 4.25 (dd, 0.4H), 3.94 (t, 0.8H, J=8.0 Hz, J=8.4 Hz), 3.87-3.81 (m, 0.8H), 3.79-3.71 (m, 0.4H), 3.64-3.51 (m, 0.8H), 3.46-3.43 (m, 0.8H). HRMS (ESI⁺): m/z [M+Na]⁺ calcd for C₃₆H₂₅C₁₄F₃N₂O₇Na, 817.0266; found 817.0242.

Allyl 3-O-benzyl-4,6-O-benzylidene-2-deoxy-2-tetrachlorophthalimido-β-L-altropyranosyl-(1→3)-4-azido-2,4,6-trideoxy-2-trichloroacetamido-β-D-galactopyranoside (17), Allyl 3-O-benzyl-4,6-O-benzylidene-2-deoxy-2-tetrachlorophthalimido-α-L-altropyranosyl-(1-3)-4-azido-2,4,6-trideoxy-2-trichloroacetamido-β-D-galactopyranoside (18) and 3-O-Benzyl-4,6-O-benzylidene-2-deoxy-2-tetrachlorophthalimido-L-altral (S1). A mixture of acceptor 8 (600 mg, 1.61 mmol, 1.0 equiv.) and PTFA donor 16 (1.4 g, 1.77 mmol, 1.1 equiv.) in anhyd. DCE (20 mL) was stirred with freshly activated 4 Å MS (1.0 g) for 30 min at rt under an Ar atmosphere. The reaction mixture was cooled to −20° C. and TMSOTf (19 μL, 10 μmol, 0.06 equiv.) was added slowly. After stirring for 30 min at −20° C., at which time a TLC follow up indicated donor consumption, Et₃N was added, the suspension was filtered and the filtrate was concentrated in vacuo. Flash chromatography using cHex/EtOAc (15:1→10:1) gave the unwanted β-isomer 17 (960 mg, 0.98 mmol, 61%) as a white solid, along with the desired α-isomer 18 (150 mg, 0.15 μmol, 10%), and the elimination product S1 (220 mg, 0.36 mmol, 20% wrt 16). The β-isomer 17 had R_{f} 0.35 (cHex/EtOAc 10:1). ¹H NMR (CDCl₃) δ 7.53-7.39 (m, 5H, H_{Ar}), 7.09 (dd, 2H, J=1.0, 8.0 Hz, H_{Ar}), 6.95 (t, 2H, J=7.6 Hz, H_{Ar}), 6.79-6.73 (m, 2H, H_{Ar}, NH), 6.20 (d, 1H, J_{1,2}=8.5 Hz, H-1_{A}), 5.90-5.80 (m, 1H, CH_{All}), 5.58 (s, 1H, H_{Bzl}), 5.28-5.23 (m, 1H, CH_{2All}), 5.18-5.14 (m, 1H, CH_{2All}), 4.82 (d, 1H, J=12.5 Hz, CH_{2Bn}), 4.77 (d, 1H, J_{1,2}=8.4 Hz, H-1_{B}), 4.45 (dd, 1H, J_{5,6a}=5.1 Hz, J_{6a,6b}=10.2 Hz, H-6a_{A}), 4.41 (ddₚₒ, 1H, J_{3,4}=3.4 Hz, J_{2,3}=10.9 Hz, H-3_{B}), 4.40 (dₚₒ, 1H, CH_{2Bn}), 4.35-4.30 (m, 1H, CH_{2All}), 4.28 (brt, H-3_{A}), 4.24 (ddd, 1H, J_{4,5}=9.6 Hz, J_{5,6}=4.8 Hz, H-5_{A}), 4.22 (dd, 1H, J_{2,3}=3.1 Hz, H-2_{A}), 4.07-4.02 (m, 1H, CH_{2All}), 3.97 (brd, 1H, H-4_{B}), 3.86 (t, 1H, J_{5,6b}=10.4 Hz, H-6_{bA}), 3.80 (dd, 1H, J_{4,5}=9.6 Hz, J_{3,4}=2.3 Hz, H-4_{A}), 3.72-3.64 (m, 2H, H-2_{B}, H-5_{B}), 1.36 (d, 3H, J_{5,6}=6.4 Hz, H-6_{B}). ¹³C NMR (CDCl₃) δ 163.8 (CO_{NHTCA}), 162.7, 161.6 (2C, CO_{NTCP}), 139.9, 139.5, 137.3, 137.1 (C_{q, Ar}), 133.5 (CH_{All}), 129.6, 129.3, 129.2, 128.9, 128.3, 128.0, 127.7, 127.1, 126.2, 126.1 (C_{Ar}), 117.7 (CH_{2All}), 102.0 (C_{Bzl}), 98.8 (C-1_{B}, ¹J_{C,H}=161 Hz), 96.0 (C-1_{A}, ¹J_{C,H}=173 Hz), 92.8 (CCl₃), 79.2 (C-4_{A}), 76.9 (C-3_{B}), 74.6 (CH_{2Bn}), 73.9 (C-3_{A}), 69.9 (CH_{2All}), 69.3 (C-5_{B}), 69.2 (C-6_{A}), 64.5 (C-5_{A}), 63.5 (C-4_{B}), 57.1 (C-2_{A}), 53.7 (C-2_{B}), 17.4 (C-6_{B}). HRMS (ESI⁺): m/z [M+Na]⁺ calcd for C₃₉H₃₄C₁₇N₅O₁₀Na, 1000.0023; found 1000.0015.

The α-isomer 18 had R_{f} 0.3 (cHex/EtOAc 10:1). ¹H NMR (CDCl₃) δ 7.54-7.51 (m, 2H, H_{Ar}), 7.48-7.37 (m, 3H, HA), 7.28 (d, 2H, J=6.8 Hz, H_{Ar}), 7.12 (tₚₒ, 3H, J=8.0 Hz, H_{Ar}), 6.97 (tₚₒ, 1H, J=7.2 Hz, H_{Ar}), 6.79 (d, 1H, J_{2,NH}=6.4 Hz, NH_{B}), 5.86-5.76 (m, 1H, CH_{All}), 5.63 (s, 1H, H_{Bzl}), 5.35 (d, 1H, J_{1,2}=5.6 Hz, H-1_{A}), 5.24-5.18 (m, 1H, CH_{2All}), 5.16-5.12 (m, 1H, CH_{2All}), 4.82 (d, 1H, J=12.0 Hz, CH_{2Bn}), 4.81 (d, 1H, J_{1,2}=8.0 Hz, H-1_{B}), 4.74 (dd, 1H, J_{2,3}=4.8, Hz, H-2_{A}), 4.64 (dd, 1H, J_{3,4}=3.6 Hz, J_{2,3}=10.8 Hz, H-3_{B}), 4.58-4.46 (m, 2H, H-5_{A}, CH_{2Bn}), 4.50 (dd, 1H, J_{5,6a}=5.2 Hz, J_{6a,6b}=10.4 Hz, H-6a_{A}), 4.32-4.27 (m, 1H, CH_{2All}), 4.21 (dd, 1H, J_{3,4}=4.8 Hz, J_{4,5}=8.8 Hz, H-4_{A}), 4.12 (brt, 1H, H-3_{A}), 4.02-3.97 (m, 1H, CH_{2All}), 3.89 (brd, 1H, J_{3,4}=2.8 Hz, H-4_{B}), 3.85 (t, 1H, J_{5,6b}=10.1 Hz, H-6b_{A}), 3.71 (dqₚₒ, 1H, H-5_{B}), 3.53-3.46 (m, 1H, H-2_{B}), 1.39 (d, 3H, J_{5,6}=6.4 Hz, H-6_{B}). ¹³C NMR (CDCl₃) δ 162.5 (CO_{NHTCA}), 161.7 (CO_{NTCP}), 140.3, 137.9, 137.3 (C_{q,Ar}), 133.4 (CH_{All}), 129.9, 129.1, 128.4, 128.2, 127.9, 127.1, 126.9, 126.2 (C_{Ar}), 118.0 (CH_{2All}), 101.7 (C_{Bzl}), 97.7 (C-1_{A}, ¹J_{C,H}=173 Hz), 97.3 (C-1_{B}, ¹J_{C,H}=162 Hz), 92.1 (CCl₃), 76.3 (C-4_{A}), 76.1 (C-3_{B}), 73.0 (CH_{2Bn}), 72.0 (C-3_{A}), 70.2 (CH_{2All}), 69.6 (C-6_{A}), 69.1 (C-5_{B}), 65.8 (C-4_{B}), 60.8 (C-5_{A}), 55.7 (C-2_{A}), 55.7 (C-2_{B}), 17.5 (C-6_{B}). HRMS (ESI⁺): m/z [M+NH₄]⁺ calcd for C₃₉H₃₈Cl₇N₆O₁₀, 995.0469; found 995.0437.

Altral S1 had R_{f} 0.65 (cHex/EtOAc 10:1). ¹H NMR (CDCl₃) δ 7.58-7.56 (m, 2H, H_{Ar}), 7.45-7.40 (m, 3H, H_{Ar}), 7.15 (dₚₒ, 2H, J=7.2 Hz, H_{Ar}), 6.96-6.92 (mₚₒ, 2H, H_{Ar}), 6.79-6.75 (m, 1H, H_{Ar}), 6.61 (s, 1H, H-1), 5.67 (s, 1H, H_{Bzl}), 4.83 (d, 1H, J=12.6 Hz, CH_{2Bn}), 4.60 (dd, 1H, J_{5,6a}=5.3 Hz, J_{6a,6b}=10.4 Hz, H-6a), 4.50 (dt, 1H, J_{5,6b}=10.3 Hz, J_{4,5}=5.3 Hz, H-5), 4.39 (d, 1H, J_{3,4}=5.3 Hz, H-3), 4.38 (d, 1H, CH_{2Bn}), 4.21 (dd, 1H, H-4), 3.96 (t, 1H, H-6b). ¹³C NMR (CDCl₃) δ 162.5 (CO_{NTCP}), 147.3 (C-1), 139.9, 138.6, 137.2 (C_{q,Ar}), 129.7, 129.2, 128.7, 128.3, 127.9, 127.1, 126.9, 126.2 (C_{Ar}), 108.4 (C-2), 101.7 (C_{Bzl}), 77.9 (C-4), 74.0 (CH_{2Bn}), 68.3 (C-6), 67.6 (C-3), 65.1 (C-5). HRMS (ESI⁺): m/z [M+NH₄]⁺ calcd for C₂₈H₂₃C₁₄N₂O₆, 623.0310; found 623.0295.

3-O-Benzyl-4,6-O-benzylidene-2-deoxy-2-N-(9-fluorenylmethoxycarbonyl)-α/β-L-altropyranosyl (N-phenyl)trifluoroacetimidate (19). Hemiacetal 15 (170 mg, 293 μmol, 1.1 equiv.) was dissolved in acetone (6 mL). PTFACl (60 μL, 382 μmol, 1.3 equiv.) was added followed by addition of Cs₂CO₃ (105 mg, 323 μmol, 1.1 equiv.). After stirring at rt for 1 h, a TLC analysis indicated reaction completion. The reaction mixture was filtered over a pad of Celite, and solids were washed with DCM (5 mL) twice. Volatiles were evaporated under reduced pressure and the residue was purified by flash chromatography eluting with cHex/EtOAc (98:2→90:10) to give the desired 19 (200 mg, 273 μmol, 90%) as an off-white solid. The constrained PTFA donor had R_{f} 0.8 (Tol/EtOAc 9:1). ¹H NMR (cc anomer, CDCl₃) δ 7.68-7.50 (m, 18H, H_{Ar}), 5.67 (d, 1H, J=3.6 Hz, H-1), 5.50 (s, 1H, H_{Bzl}), 4.67 (brs, 1H, CH_{2Fmoc}), 4.54 (brs, 1H, CH_{2Bn}), 4.50 (ddₚₒ, 1H, CH_{2Fmoc}), 437-4.33 (m, 1H, H-6a), 4.27-4.10 (m, 3H, H-5, H-3, CH_{Fmoc}), 3.70-3.58 (m, 2H, H-6b, H-2) 3.60 (ddₚₒ, 1H, J_{3,4}<1.0 Hz, J_{4,5}=8.8 Hz, H-4). ¹³C NMR (CDCl₃) δ 153.4 (CO_{NFmoc}), 141.3, 139.7, 137.8, 137.4 (C_{q,Ar}), 129.4, 129.1, 128.5, 128.3, 128.2 (2C), 127.8, 127.7, 127.6, 127.5, 127.3, 127.2, 127.0, 126.3, 126.1, 124.6, 124.3, 122.5, 120.4, 120.3, 120.2 (C_{Ar}), 102.6 (C_{Bzl}), 95.8 (C-1_{A}, ¹J_{C,H}=182 Hz), 75.7 (C-4_{α}), 73.7 (CH_{2Bn}), 71.6 (C-3), 69.0 (C-6), 67.2 (CH_{2Fmoc}), 63.7 (C-5_{α}), 61.2 (C-2_{α}), 47.0 (CH_{Fmoc}). HRMS (ESI⁺): m/z [M+H]⁺ calcd for C₄₃H₃₈F₃N₂O₇, 751.2631; found 751.2665.

Allyl 3-O-benzyl-4,6-O-benzylidene-2-deoxy-2-N-(9-fluorenylmethoxycarbonyl)-α-L-altropyranosyl-(1→3)-4-azido-2,4,6-trideoxy-2-trichloroacetamido-β-D-galactopyranoside (21). Hemiacetal 15 (343 mg, 59 μmol, 1.1 equiv.) was dissolved in acetone (10 mL) and cooled to 0° C. Trichloroacetonitrile (855 μL, 2.36 mmol, 4.0 equiv.) was added followed by addition of K₂CO₃ (163 mg, 1.18 mmol, 2.0 equiv.). After stirring for 4 h at 0° C., the reaction mixture was filtered over a pad of Celite, washed with DCM (5 mL) twice. The filtrate was concentrated under reduced pressure to give the crude trichloroacetimidate 20.

The crude 20 (1.1 equiv. theo) was mixed with acceptor 8 (200 mg, 538 μmol, 1.0 equiv.), coeveporated with toluene repeatedly, and dried under high vacuum for 2 h. The mixture was dissolved in anhyd. DCM (10 mL) and stirred with freshly activated MS 4 Å (500 mg) for 45 min under an Ar atmosphere before the temperature was set to −15° C. TMSOTf (7 μL, 30 μmol, 0.05 equiv.) was added slowly. After stirring at this temperature for 30 min, a TLC analysis (Tol/EtOAc 3:1) showed the presence of a new spot (R_{f} 0.4) close to those featuring acceptor 8 (R_{f} 0.35) and hemiacetal 15 (R_{f} 0.32). Et₃N (˜10 μL) was added and the suspension was filtered over a fitted funnel. The filtrate was concentrated under reduced pressure and the residue was purified by flash chromatography eluting with Tol/EtOAc (5:1→4:1) to give the desired disaccharide 21 (200 mg, 214 μmol, 62%). The coupling product had ¹H NMR (CDCl₃) δ 7.80-7.77 (m, 2H, H_{Ar}), 7.59-7.57 (dₚₒ, 2H, H_{Ar}), 7.52-7.29 (m, 15H, H_{Ar}), 6.90 (d, J_{2,NH}=6.2 Hz, NH_{B}), 5.90-5.80 (m, 1H, CH_{All}), 5.56 (s, 1H, CH_{Bzl}), 5.29-5.24 (m, 1H, CH_{2All}), 5.20-5.17 (m, 1H, CH_{2All}), 4.99 (d, 1H, J_{1,2}=8.0 Hz, H-1_{B}), 4.87 (dₚₒ, 1H, J=11.8 Hz, CH_{2Bn}), 4.84 (dₚₒ, 1H, J=7.2 Hz, NH_{A}), 4.80 (dₚₒ, 1H, CH_{2Bn}), 4.77 (sₚₒ, 1H, H-1_{A}), 4.64-4.58 (m, 2H, H-3_{B}, H-5_{A}), 4.52 (brd, 2H, J=5.8 Hz, CH₂, NHFmoc), 4.36-4.31 (m, 2H, H-6a_{A}, CH_{2All}), 4.24 (brd, 1H, J_{2,NH}=7.2 Hz, H-2_{A}), 4.20 (t, 1H, J=6.0 Hz, CH_{NHFmoc}), 4.09-4.04 (m, 1H, CH_{2All}), 3.87 (brs, 1H, H-3_{A}), 3.78 (brq, 1H, H-5_{B}), 3.72 (t, 1H, J_{5,6b}=J_{6a,6b}=10.5 Hz, H-6b_{A}), 3.69 (brdₚₒ, 1H, J_{3,4}=3.0 Hz, H-4_{B}), 3.67 (brdₚₒ, 1H, J_{4,5}=8.0 Hz, H-4_{A}), 3.49-3.43 (m, 1H, H-2_{B}), 1.42 (d, 3H, J_{5,6}=6.4 Hz, H-6_{B}). ¹³C NMR (CDCl₃) δ 162.2 (CO_{NTCA}), 154.9 (CO_{NFmoc}), 143.5, 141.4, 141.3, 138.9, 137.3 (C_{q,Ar}), 133.5 (CH_{All}), 129.1, 128.2, 128.0, 127.8, 127.4, 127.2, 127.1, 126.2, 124.8, 124.7, 120.0 (C_{Ar}), 117.9 (CH_{2All}), 102.3 (C_{Bzl}), 101.6 (C-1_{A}, ¹J_{C,H}=169 Hz), 97.2 (C-1_{B}, ¹J_{C,H}=162 Hz), 92.0 (CCl₃), 76.7 (C-4_{A}), 75.6 (C-3_{B}), 74.0 (C-3_{A}), 72.3 (CH_{2Bn}), 70.2 (CH_{2All}), 69.7 (C-5_{B}), 69.1 (C-6_{A}), 66.6 (CH_{2Fmoc}), 65.9 (C-4_{B}), 59.7 (C-5_{A}), 56.1 (C-2_{B}), 52.4 (C-2_{A}), 47.3 (CH_{NHFmoc}), 17.4 (C-6_{B}). HRMS (ESI⁺): m/z [M+NH₄]⁺ calcd for C₄₆H₅₀Cl₃N₆O₇, 951.2654; found 951.2694.

Use of a A Donor Whereby Protecting Groups as the 4_{A}-OH and 6_{A}-OH are Identical

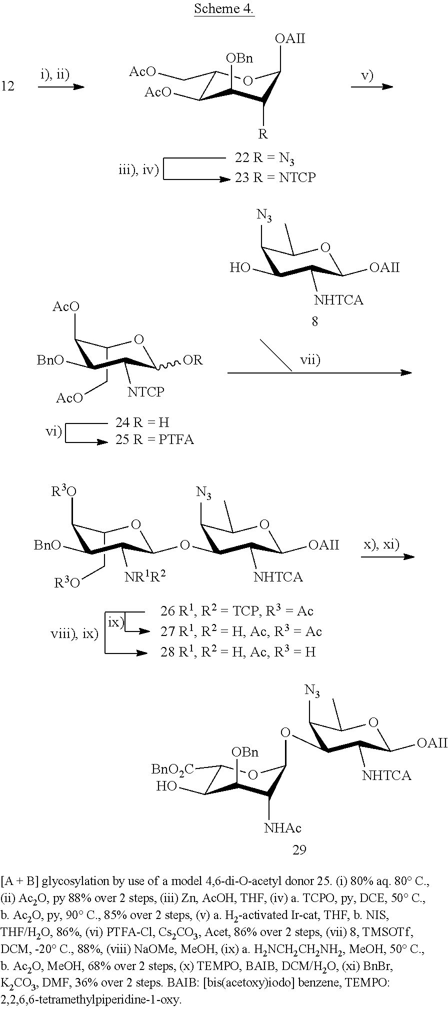

Allyl 4,6-di-O-acetyl-3-O-benzyl-2-deoxy-2-tetrachlorophthalimido-α-L-altropyranoside (23). Zn dust (5.6 g, 85.8 mmol, 8.0 equiv.) and AcOH (4.9 mL, 85.8 mmol, 8.0 equiv.) were added to a solution of azide 22 (4.5 g, 10.7 mmol, 1.0 equiv.) in THF (70 mL) at rt. The reaction mixture was stirred for 2 h at which point a TLC follow up (Tol/EtOAc 1:1) revealed the absence of any remaining 22 (R_{f} 0.8) and the presence of a more polar product. The mixture was filtered over a pad of Celite and solids were washed with DCM (100 mL) twice. The combined organic phases were washed with satd. aq. NaHCO₃ (200 mL) and brine (200 mL). The organic phase was dried over Na₂SO₄, filtered, and concentrated. The residue was dried under high vacuum for 3 h and subjected as such to the next step. The crude amine intermediate had HRMS (ESI⁺): m/z [M+Na]⁺ calcd for C₂₀H₂₈NO₇, 394.1866; found 394.1856.

Tetrachlorophthalic anhydride (3.68 g, 12.8 mmol, 1.2 equiv.) was added to a solution of the crude intermediate in DCM (40 mL) and the solution was stirred for 30 min at rt. Et₃N (1.79 mL, 12.8 mmol, 1.2 equiv.) was added and the reaction mixture was stirred for another 30 min. Volatiles were eliminated under reduced pressure and the residue was dried under high vacuum for 1 h. The crude material was dissolved in pyridine (50 mL) and Ac₂O (5.0 mL, 53.6 mmol, 5.0 equiv.) was added at 0° C. The mixture was heated to 80° C. for 10 min. A TLC follow up (Tol/EtOAc 9:1) showed the formation of a product (R_{f} 0.7) slightly more polar than azide 22 (0.75). After it reached rt, the reaction mixture was concentrated and coevaporated with Toluene (15 mL) twice. The residue was diluted with DCM (100 mL) and washed with 1N aq. HCl (200 mL), satd. aq. NaHCO₃ (200 mL) and brine (200 mL). The DCM layer was dried over Na₂SO₄, filtered, concentrated and the residue was purified by flash chromatography eluting with cHex/EtOAc (12:1→9:1) to give diacetate 23 as a yellowish foam (6.0 g, 9.1 mmol, 85%). Compound 23 had R_{f} 0.35 (Tol/EtOAc 10:1). ¹H NMR (CDCl₃) δ 7.29-7.17 (m, 2H, H_{Ar}), 7.06-7.00 (m, 3H, H_{Ar}), 5.81-5.72 (m, 1H, CH_{All}), 5.50 (ddₚₒ, 1H, H-4), 5.37 (d, 1H, J=7.3 Hz, H-1), 5.22-5.17 (m, 1H, CH_{2All}), 5.13-5.09 (m, 1H, CH_{2All}), 4.61 (d, 1H, J=12.4 Hz, CH_{2Bn}), 4.53 (dd, 1H, Hz, J_{2,3}=11.2 Hz, H-2), 4.44 (dd, 1H, J_{3,4}=4.3 Hz, H-3), 4.39 (dd, 1H, J_{5,6a}=6.6 Hz, J_{6a,6b}=11.7 Hz, H-6a), 4.37 (dd, 1H, J_{5,6b}=5.7 Hz, H-6b), 4.30 (dtₚₒ, 1H, J_{4,5}=3.1 Hz, H-5), 4.23-4.18 (m, 1H, CH_{2All}), 4.20 (dₚₒ, 1H, CH_{2Bn}), 4.01-3.96 (m, 1H, CH_{2All}), 2.21 (s, 3H, CH_{3Ac}), 2.17 (s, 3H, CH_{3Ac}). ¹³C NMR (CDCl₃) δ 170.0 (CO_{Ac}), 162.9 (CO_{NTCP}), 139.9, 137.4, 129.6, 127.0, 125.2 (C_{q,Ar}), 133.4 (CH_{All}), 129.0, 128.2, 128.1, 127.6 (C_{Ar}), 117.7 (CH_{2All}), 95.4 (C-1, ¹J_{C,H}=169 Hz), 72.7 (C-5), 72.1 (CH_{2Bn}), 71.1 (C-3), 69.3 (CH_{2All}), 68.8 (C-4), 62.9 (C-6), 53.2 (C-2). HRMS (ESI⁺): m/z [M+NH₄]⁺ calcd for C₂₈H₂₉Cl₄N₂O₉, 677.0627; found 677.0622.

4,6-Di-O-acetyl-3-O-benzyl-2-deoxy-2-tetrachlorophthalimido-α/β-L-altropyranose (24). [Ir(COD)(PMePh₂)₂]PF₆ (59 mg, 0.07 mmol, 0.02 equiv.) was stirred in anhyd. THF (5.0 mL) under an H₂ atmosphere at rt for 30 min. The resulting yellow solution was degassed several times with Ar and transferred by use of a cannula into a solution of allyl glycoside 23 (2.3 g, 3.4 mmol, 1.0 equiv.) in anhyd. THF (25 mL). After stirring at rt for 1 h, NIS (864 mg, 3.8 mmol, 1.05 equiv.) and H₂O (5 mL) were added. After 2 h, a TLC analysis (Tol/EtOAc 4:1) revealed the presence of a compound (R_{f} 0.35) more polar that allyl glycoside 23 (R_{f} 0.65). 10% Aq. Na₂SO₃ was added and the reaction mixture was concentrated to remove the THF and the aq. phase was extracted with DCM (50 mL) thrice. The combined DCM phases were washed with brine, dried over Na₂SO₄, filtered, and concentrated under reduced pressure. The residue was purified by flash chromatography using Tol/EtOAc (8:1→6:1) to give the expected 24 (2.0 g, 3.2 mmol, 92%) as a white floppy solid (α/β˜5:1). Hemiacetal 24 (α anomer) had ¹H NMR (extracted, CDCl₃) δ 7.28-7.01 (m, 5H, H_{Ar}), 5.61 (t, 1H, J_{1,2}=J_{1,OH}=7.6 Hz, H-1), 5.60 (dd, 1H, H-4), 4.60 (d, 1H, J=12.4 Hz, CH_{2Bn}), 4.53 (dd, 1H, J_{3,4}=3.8 Hz, J_{2,3}=11.1 Hz, H-3), 4.43-4.35 (m, 3H, H-2, H-6a, H-6b), 4.32 (ddd, 1H, J_{4,5}=2.1 Hz, J_{5,6}=5.1 Hz, J_{5,6}=7.1 Hz, H-5), 4.22 (d, 1H, CH_{2Bn}), 3.49 (d, 1H, OH). ¹³C NMR (CDCl₃) δ 170.6, 170.4 (CO_{Ac}), 163.1 (CO_{NTCP}), 140.0, 137.8, 137.4 (C_{q,Ar}), 129.7, 129.0, 128.1 (2C), 127.7 (C_{Ar}), 128.3, 128.2, 127.8, 125.3, 90.4 (C-1_{A}, ¹J_{C,H}=170 Hz), 73.8 (C-3), 71.9 (CH_{2Bn}), 70.8 (C-5), 66.9 (C-4), 62.6 (C-6), 55.0 (C-2), 21.0, 20.8 (2C, CH_{3Ac}). HRMS (ESI⁺): m/z [M+NH₄]⁺ calcd for C₂₅H₂₅C₁₄N₂O₉, 637.0314; found 637.0336.

Hemiacetal 24 (β anomer) had ¹H NMR (extracted, CDCl₃) δ 7.28-7.01 (m, 5H, H_{Ar}), 5.64 (dd, 1H, J_{3,4}=2.0 Hz, J_{4,5}=2.8 Hz, H-4), 5.43 (t, 1H, J_{1,2}=J_{1,OH}=4.0 Hz, H-1), 5.21 (dd, 1H, J_{5,6a}=3.2 Hz, J_{5,6b}=11.2 Hz, H-5), 4.75 (dd, 1H, J_{1,2}=3.2 Hz, J_{2,3}=10.4 Hz, H-2), 4.69 (d, 1H, J=10.8 Hz, CH_{2Bn}), 4.60 (d, 1H, J=12.4 Hz, CH_{2Bn}), 4.54-4.49 (mₒ, 2H, H-6a, H-6b), 4.43-4.35 (mₒ, 1H, CH_{2Bn,β}), 4.21-4.17 (mₚₒ, 1H, H-3), 4.11 (d, 1H, OH). ¹³C NMR (CDCl₃), δ 170.9, 170.3 (CO_{Ac}), 163.8 (CO_{NTCP}), 140.2, 137.8 (C_{q,Ar}), 128.3, 128.2, 127.8, 125.2 (C_{Ar}), 93.1 (C-1_{A}, ¹J_{C,H}=175 Hz), 75.2 (C-3), 71.5 (CH_{2Bn}), 67.6 (C-5), 67.5 (C-4), 64.5 (C-6), 53.0 (C-2), 21.4, 21.0 (2C, CH_{3Ac}). HRMS (ESI⁺): m/z [M+NH₄]⁺ calcd for C₂₅H₂₅C₁₄N₂O₉, 637.0314; found 637.0336.

4,6-Di-O-acetyl-3-O-benzyl-2-deoxy-2-tetrachlorophthalimido-α/β-L-altropyranosyl (N-phenyl)trifluoroacetimidate (25). Hemiacetal 24 (1.5 g, 2.4 mmol, 1.0 equiv.) was dissolved in acetone (20 mL). PTFACl (580 μL, 3.6 mmol, 1.5 equiv.) was added followed by the addition of cesium carbonate (947 mg, 2.9 mmol, 1.2 equiv.). The reaction mixture was stirred at rt. After 2 h, a TLC analysis (Tol/EtOAc 6:1) showed the presence of a new compound (R_{f} 0.65) and the absence of hemiacetal 24 (R_{f} 0.2). The reaction mixture was filtered through a pad of Celite and solids were washed with acetone (10 mL) twice. The filtrate was concentrated and the residue was purified by flash chromatography (cHex/EtOAc 9:1) to give PTFA 25 as a white solid (1.8 g, 2.2 mmol, 94%). Donor 25 (a/P 3:2) had R_{f} 0.65 (cHex/EtOAc 9:1). ¹H NMR (CDCl₃) δ 7.29-6.99, 6.72, 6.54 (m, 14H, H_{Ar}), 6.54, (bsₒ, 0.6H, H-1_{α}), 6.43 (brs, 0.4H, H-1_{β}), 5.71 (d, 0.4H, J_{4,5}=2.4 Hz, H-4β), 5.54 (dd, 0.6H, J_{3,4}=3.2 Hz, J_{4,5}=3.6 Hz, H-4a), 5.45 (dd, 0.4H, J_{3,4}=2.8 Hz, J_{2,3}=9.6 Hz, H-3β), 4.97 (dd, 0.4H, J_{1,2}=3.6 Hz, H-2β), 4.71 (d, 0.4H, J=10.4 Hz, CH_{2Bnβ}), 4.7 (dd, 0.6H, J_{1,2}=4.8 Hz, J_{2,3}=10.8 Hz, H-2α), 4.65 (d, 0.6H, J=12.4 Hz, CH_{2Bnα}), 4.60 (ddₚₒ, 0.4H, J_{6a,6b}=8.0 Hz, J_{5,6b}=4.4 Hz, H-6a_{β}), 4.65 (d, 0.6H, CH_{2Bnα}), 4.51-4.35 (m, 2.2H, H-5_{α}, H-5_{β}, H-6a_{α}, H-6 b_{β}, H-3_{α}), 4.33 (dd, 0.6H, J_{6a,6b}=12.4 Hz, J_{5,6b}=4.8 Hz, H-6b_{α}), 4.23 (d, 0.6H, CH_{2Bnα}), 2.23, 2.16 (2s, 1.8H, CH_{3Acα}), 2.21, 2.17 (2s, 1.2H, CH_{3Acβ}). ¹³C NMR (CDCl₃) δ 170.4 (2C, CO_{Ac,β}), 170.3, 169.9 (2C, CO_{Ac,α}), 163.2 (CO_{TCP,β}), 162.6 (CO_{TCP,α}), 142.9, 142.5, 140.3, 137.8, 129.8, 126.9, 124.5 (C_{Ar, β, q}), 140.1, 137.2, 129.7, 126.9, 124.5 (C_{Ar, α, q}), 129.0, 128.6 (2C), 128.3, 128.2, 128.1, 127.9, 127.7, 125.2, 124.5 (2C), 119.4, 119.1 (C_{Ar, β}, C_{Ar, α}), 95.2 (C-1_{A}, ¹J_{C,H}=182 Hz, C-1_{β}, ¹J_{C,H}=177 Hz), 76.3 (C-5_{β}), 74.2 (C-5_{α}), 72.3 (CH_{2Bnα}), 71.8 (CH_{2Bnβ}), 70.8 (C-3_{β}), 67.5 (C-4_{α}, C-4_{β}), 66.7 (C-3_{α}), 63.3 (C-6_{β}), 63.2 (C-6_{α}), 52.3 (C-2_{α}), 51.5 (C-2_{β}), 21.4, 20.9 (CH_{3Acα}), 20.8, 20.6 (CH_{3Acβ}).

Allyl 4,6-di-O-acetyl-3-O-benzyl-2-deoxy-2-tetrachlorophthalimido-α-L-altropyranosyl-(1→3)-4-azido-2,4,6-trideoxy-2-trichloroacetamido-β-D-galactopyranoside (26). A mix of acceptor 8 (2.03 g, 5.45 mmol, 1.0 equiv.) and PTFA 25 (5.4 g, 6.54 mmol, 1.2 equiv.) was coevaporated with anhyd. toluene (20 mL), dried under high vacuum for 1 h, and then dissolved in anhyd. DCE (130 mL). Freshly activated MS 4 Å (1.0 g) was added and after stirring for 45 min at rt under an Ar atmosphere, the reaction mixture was cooled to 0° C. and TMSOTf (59 μL, 327 μmmol, 0.05 equiv.) was added. After 20 min at 0° C., a TLC follow up (Tol/EtOAc 3:1) showed a new spot (R_{f} 0.4) and no remaining acceptor 8 (R_{f} 0.35). Et₃N was added and the suspension was passed through a fitted funnel. Solids were washed DCM (30 mL) twice and the filtrate was concentrated under reduced pressure. Flash chromatography using Tol/EtOAc (8:1→6:1) yielded the desired disaccharide 26 (4.7 g, 4.83 mmol, 88%) as a white solid. Diacetate 26 had ¹H NMR (CDCl₃) δ 7.28-7.16 (m, 2H, H_{Ar}), 7.00 (brs, 3H, H_{Ar}), 6.67 (d, 1H, J_{2,NH}=6.8 Hz, NH_{B}), 5.83-5.74 (m, 1H, CH_{All}), 5.56 (d, 1H, J_{1,2}=7.6 Hz, H-1_{A}), 5.46 (dd, 1H, J_{4,5}=2.8 Hz, H-4_{A}), 5.22-5.16 (m, 1H, CH_{2All}), 5.17-5.10 (m, 1H, CH_{2All}), 4.75 (d, 1H, J_{1,2}=8.3 Hz, H-1_{B}), 4.58 (ddₚₒ, 1H, J_{2,3}=10.8 Hz, J_{3,4}=3.6 Hz, H-3_{B}), 4.57 (do, 1H, CH_{2Bn}), 4.57 (ddₚₒ, 1H, J_{2,3}=11.2 Hz, H-2_{A}), 4.41 (ddₚₒ, 1H, J_{3,4}=3.9 Hz, H-3_{A}), 4.40-4.35 (m, 3H, H-5_{A}, H-6a_{A}, H-6b_{A}), 4.29-4.24 (m, 1H, CH_{2All}), 4.16 (d, 1H, J=12.4 Hz, CH_{2Bn}), 4.00-3.95 (m, 1H, H-4_{B}, CH_{2All}), 3.68 (dq, J_{4,5}=1.0 Hz, H-5_{B}), 3.55 (ddd, 1H, J_{2,3}=11.0 Hz, H-2_{B}), 2.18 (s, 3H, CH_{3Ac}), 2.17 (s, 3H, CH_{3Ac}), 1.37 (d, 3H, J_{5,6}=6.4 Hz, H-6_{B}). ¹³C NMR (CDCl₃) δ 170.5, 170.3 (CO_{Ac}), 162.9 (CO_{NHTCA}), 161.5 (CO_{NTCP}), 139.9, 137.8, 137.2 (C_{q, Ar}), 133.4 (CH_{All}), 129.0, 128.2, 128.1, 128.0, 127.6, 125.2 (C_{Ar}), 118.0 (CH_{2All}), 97.9 (C-1_{A}, ¹J_{C,H}=171 Hz), 97.5 (C-1_{B}, ¹J_{C,H}=162 Hz), 92.2 (CCl₃), 76.6 (C-3_{B}), 73.2 (C-3_{A}), 72.1 (CH_{2Bn}), 70.6 (C-5_{A}), 70.1 (CH_{2All}), 69.0 (C-5_{B}), 66.5 (C-4_{A}), 65.4 (C-4_{B}), 62.8 (C-6_{A}), 55.3 (C-2_{B}), 53.1 (C-2_{A}), 20.8 (2C, CH_{3Ac}), 17.5 (C-6_{B}). HRMS (ESI⁺): m/z [M+NH₄]⁺ calcd for C₃₆H₃₈C₁₇N₆O₁₂, 991.0367; found 991.0355.

Allyl (2-acetamido-4,6-di-O-acetyl-3-O-benzyl-2-deoxy-α-L-altropyranosyl)-(1→3)-4-azido-2,4,6-trideoxy-2-trichloroacetamido-β-D-galactopyranoside (27). Ethylenediamine (41 μL, 617 μmol, 4.0 equiv.) was added to disaccharide 24 (150 mg, 154 μmol, 1.0 equiv.) in n-butanol (8 mL) and the solution was heated at 70° C. for 72 h. A TLC follow up (Tol/EtOAc 4:1) indicated the absence of phtalimide 24 and after the reaction mixture reached rt, volatiles were eliminated and the residue was coevaporated with toluene (5 mL) twice. Acetic anhydride (0.15 mL, 1.5 mmol, 10 equiv.) was added to the residue in pyridine (4 mL) and the system was stirred at rt. In the absence of further evolution after for 4 h (TLC:Tol/EtOAc 4:1), volatiles were evaporated and the residue was purified by column chromatography eluting with Tol/EtOAc (80:20→70:30) to give 27 (76 mg, 101 μmol, 65%) as a white solid. The desired 27 had R_{f} 0.45 (Tol/EtOAc 4:1). ¹H NMR (CDCl₃) δ 7.41-7.30 (m, 5H, H_{Ar}), 6.67 (d, 1H, J_{2,NH}=8.3 Hz, NH_{B}), 5.83-5.74 (m, 1H, CH_{All}), 5.56 (d, 1H, J_{1,2}=7.6 Hz, NH_{A}), 5.29-5.24 (m, 1H, CH_{2All}), 5.20-5.17 (m, 1H, CH_{2All}), 5.02 (dd, 1H, J_{3,4}=3.4 Hz, J_{4,5}=8.2 Hz, H-4_{A}), 4.90 (d, 1H, J_{1,2}=2.2 Hz, H-1_{A}), 4.87 (d, 1H, J_{1,2}=8.4 Hz, H-1_{B}), 4.67 (d, 1H, J=12.3 Hz, CH_{2Bn}), 4.64 (d, 1H, CH_{2Bn}), 4.59 (dddₚₒ, 1H, J_{5,6a}=5.6 Hz, H-5_{A}), 4.55 (ddₚₒ, 1H, J_{3,4}=3.5 Hz, H-3_{B}), 4.37-4.31 (mₚₒ, 1H, CH_{2All}), 4.44-4.34 (m, 2H, H-2_{A}, H-6a_{A}), 4.18 (dd, 1H, J_{5,6b}=2.9 Hz, J_{6a,6b}=12.0 Hz, H-6b_{A}), 4.09-4.04 (m, 1H, CH_{2All}), 3.91 (dd, 1H, J_{2,3}=5.3 Hz, H-3_{A}), 3.84 (brd, 1H, J_{3,4}=3.4 Hz, H-4_{B}), 3.77 (brq, J_{4,5}=1.0 Hz, H-5_{B}), 3.58 (ddd, 1H, J_{2,3}=10.8 Hz, H-2_{B}), 2.18, (s, 3H, CH_{3Ac}), 2.17 (s, 3H, CH_{3Ac}), 1.37 (d, 3H, J_{5,6}=6.4 Hz, H-6_{B}). ¹³C NMR (CDCl₃) δ 170.5, 170.3 (2C, CO_{Ac}), 162.9 (CO_{NHTCA}), 161.5 (CO_{NTCP}), 139.9, 137.8, 137.2, (C_{q, Ar}), 133.4 (CH_{All}), 129.0, 128.2, 128.1, 128.0, 127.6, 125.2 (C_{Ar}), 118.0 (CH_{2All}), 97.9 (C-1_{A}, ¹J_{C,H}=171 Hz), 97.5 (C-1_{B}, ¹J_{C,H}=162 Hz), 92.2 (CCl₃), 76.6 (C-3_{B}), 73.2 (C-3_{A}), 72.1 (CH_{2Bn}), 70.6 (C-5_{A}), 70.1 (CH_{2All}), 69.0 (C-5_{B}), 66.5 (C-4_{A}), 65.4 (C-4_{B}), 62.8 (C-6_{A}), 55.3 (C-2_{B}), 53.1 (C-2_{A}), 20.8 (2C, CH_{3Ac}), 17.5 (C-6_{B}). HRMS (ESI⁺): m/z [M+H]⁺ calcd for C₃₀H₃₉Cl₃N₅O₁₁, 750.1711; found 750.1746.

Allyl 2-acetamido-3-O-benzyl-2-deoxy-α-L-altropyranosyl-(1→3)-4-azido-2,4,6-trideoxy-2-trichloroacetamido-β-D-galactopyranoside (28). NaOMe (26 μL, 25% NaOMe in MeOH, 0.2 equiv.) was added to disaccharide 26 (500 mg, 514 μmol, 1.0 equiv.) in anhyd. methanol (15 mL). After stirring at rt for 1 h, a TLC follow up (Tol/EtOAc 1:2) indicated reaction completion and Dowex resin (H⁺) was added pinch by pinch to reach pH 7. The suspension was filtered by passing through a fitted funnel and the filtrate was concentrated and dried under high vacuum. The obtained diol had (HRMS (ESI⁺): m/z [M+NH₄]⁺ calcd for C₃₂H₃₄Cl₇N₆O₁₀, 907.0156; found 907.0156) Following extensive drying, the intermediate diol was dissolved in THF/MeOH (1:4, 15 mL). Ethylenediamine (137 μL, 2.0 mmol, 4.0 equiv.) was added and the mixture was heated at 70° C. for 48 h. At reaction completion (TLC:EtOAc), the reaction mixture was cooled down, concentrated under reduced pressure, and coevaporated with toluene (5 mL) twice. Et₃N (500 μL) and acetic anhydride (485 μL, 5.1 mmol, 10 equiv.) were added to the residue in methanol (10 mL). After stirring at rt for 2 h, a TLC follow up (EtOAc/MeOH 9:1) showed reaction completion. Volatiles were evaporated and the residue was purified by column chromatography (DCM/MeOH 95:5) to give the desired 28 (235 mg, 353 μmol, 69%) as a white solid. Diol 28 had R_{f} 0.2 (EtOAc). ¹H NMR (DMSO-d₆) δ 8.85 (d, 1H, J=9.0 Hz, NH_{B}), 7.93 (dₚₒ, 1H, J=8.7 Hz, NH_{A}), 7.44-7.22 (m, 5H, H_{Ar}), 5.86-5.76 (m, 1H, CH_{All}), 5.26-5.21 (m, 1H, CH_{2All}), 5.12-5.09 (m, 1H, CH_{2All}), 4.79 (d, 1H, J_{1,2}=1.7 Hz, H-1_{A}), 4.65 (d, 1H, J=12.0 Hz, CH_{2Bn}), 4.61-4.56 (m, 2H, OH), 4.51 (dₚₒ, 1H, J_{1,2}=8.3 Hz, H-1_{B}), 4.50 (dₚₒ, 1H, CH_{2Bn}), 4.27 (ddd, 1H, J_{2,3}=3.9 Hz, H-2_{A}), 4.22-4.17 (m, 1H, CH_{2All}), 4.11-4.07 (mₚₒ, 1H, J_{3,4}=3.6 Hz, H-3_{B}), 4.09 (brsₒ, 1H, H-4_{B}), 4.04 (ddd, 1H, J_{4,5}=9.2 Hz, J_{5,6a}=2.1 Hz, J_{5,6b}=7.1 Hz, H-5_{A}), 3.99-3.96 (m, 1H, CH_{2All}), 3.90-3.84 (m, 1H, H-2_{B}), 3.76-3.70 (m, 2H, H-5_{B}, H-6a_{A}), 3.64 (m, 1H, H-4_{A}), 3.54-3.48 (mₚₒ, 1H, H-6b_{A}), 3.47 (pt, 1H, J_{3,4}=4.1 Hz, H-3_{A}), 1.79 (s, 3H, CH_{3Ac}), 1.27 (d, 3H, J_{5,6}=6.4 Hz, H-6_{B}). ¹³CNMR (DMSO-d₆) δ 169.0 (CO_{NHTCA}), 162.2 (CO_{NHAc}), 139.4 (C_{q,Ar}), 134.8 (CH_{All}), 128.2, 127.9, 127.4 (C_{Ar}), 116.8 (CH_{2All}), 102.0 (C-1_{A}, ¹J_{C,H}=170 Hz), 100.3 (C-1_{B}, ¹J_{C,H}=163 Hz), 93.5 (CCl₃), 77.4 (C-3_{B}), 76.9 (C-3_{A}), 71.6 (C-5_{A}), 71.0 (CH_{2Bn}), 69.7 (C-5_{B}), 69.3 (CH_{2All}), 65.3 (C-4_{B}), 64.9 (C-4_{A}), 62.0 (C-6_{A}), 53.4 (C-2_{B}), 49.4 (C-2_{A}), 22.9 (CH_{3Ac}), 17.7 (C-6_{B}). HRMS (ESI⁺): m/z [M+H]⁺ calcd for C₂₆H₃₅Cl₃N₅O₉, 666.1486; found 666.1500.

Allyl (benzyl 2-acetamido-3-O-benzyl-2-deoxy-α-L-altropyranosyluronate)-(1→3)-4-azido-2,4,6-trideoxy-2-trichloroacetamido-β-D-galactopyranoside (29). TEMPO (89 mg, 0.057 mmol, 0.2 equiv.) and BAIB (242 mg, 0.75 mmol, 2.5 equiv.) were added to diol 28 (200 mg, 301 μmol, 1.0 equiv.) in DCM/H₂O (2:1, 15 mL). The reaction was stirred at rt for 4 h. At completion as indicated by TLC (EtOAc/MeOH 9:1), 10% aq. Na₂SO₃ was added and the biphasic mixture was diluted with DCM (20 mL). The aq. phase was separated and extracted with DCM (10 mL) twice. The aq. phase was acidified with dilute aq. HCl to reach pH 1 and washed with DCM (10 mL) thrice. The combined organic phases were washed with brine, dried by passing through a phase separator filter and concentrated under reduced pressure. Benzyl bromide (142 μL, 1.2 mmol, 4.0 equiv.) and K₂CO₃ (83 mg, 0.60 mmol, 2.0 equiv.) were added to the residue in anhyd. DMF (2 mL). After stirring at rt for 1 h, the reaction mixture was diluted with H₂O (50 mL) and the aq. layer was extracted with DCM (20 mL) thrice. The organic phases were combined, washed with brine (20 mL), dried over anhyd. Na₂SO₄, filtered and concentrated in vacuo. Flash chromatography eluting with Tol/EtOAc (3:1→2:1) gave the benzyl ester 29 (85 mg, 110 μmol, 36%) as a white solid. Disaccharide 29 had R_{f} 0.3 (Tol/EtOAc 1:1). ¹H NMR (CDCl₃) δ 7.43-7.31 (m, 10H, H_{Ar}), 6.81 (d, 1H, J_{2,NH}=7.2 Hz, NH_{B}), 5.89-5.79 (m, 1H, CH_{All}), 7.93 (d, 1H, J_{2,NH}=8.4 Hz, NH_{A}), 5.28 (d, 1H, J_{2,NH}=12.1 Hz, CH_{2Bn-6}, CH_{2All}), 5.29-5.23 (mₚₒ, 1H, CH_{2All}), 5.22 (dₚₒ, 1H, CH_{2Bn-6}), 5.20-5.16 (m, 1H, CH_{2All}), 4.93 (d, 1H, J_{1,2}=3.2 Hz, H-1_{A}), 4.78 (dₚₒ, 1H, J_{1,2}=8.4 Hz, H-1_{B}), 4.73 (d, 1H, J=11.8 Hz, CH_{2Bn}), 4.69 (dₚₒ, 1H, J_{4,5}=7.8 Hz, H-5_{A}), 4.66 (d, 1H, CH_{2Bn}), 4.51 (dd, 1H, J_{3,4}=3.7 Hz, J_{2,3}=10.7 Hz, H-3_{B}), 4.34-4.39 (m, 2H, H-2_{A}, CH_{2All}), 4.12 (ddd, 1H, J_{4,5}=7.8 Hz, H-4_{A}), 4.07-4.01 (m, 1H, CH_{2All}), 3.85 (dd, 1H, J_{2,3}=5.0 Hz, J_{3,4}=3.5 Hz, H-3_{A}), 3.80 (d, 1H, J_{3,4}=3.2 Hz, H-4_{B}), 3.61 (dq, 1H, J_{4,5}=0.9 Hz, H-5_{B}), 3.55 (ddd, J_{2,3}=10.8 Hz, 1H, H-2_{B}), 2.76 (d, 1H, J_{4,OH}=8.2 Hz, OH), 1.95 (s, 3H, CH_{3Ac}), 1.27 (d, 3H, J_{5,6}=6.4 Hz, H-6_{B}). ¹³C NMR (CDCl₃) δ 169.5 (CO_{NHTCA}), 169.4 (C-6), 162.1 (CO_{NHAc}), 137.6, 135.0 (C_{q,Ar}), 133.5 (CH_{All}), 128.7, 128.6 (2C), 128.4 (2C), 128.2, 128.0 (C_{Ar}), 117.9 (CH_{2All}), 100.6 (C-1_{A}, ¹J_{C,H}=170 Hz), 97.6 (C-1_{B}, ¹J_{C,H}=162 Hz), 92.2 (CCl₃), 76.7 (C-3_{B}), 75.2 (C-3_{A}), 72.0 (CH_{2Bn}), 71.0 (C-5_{A}), 70.0 (CH_{2All}), 69.6 (C-5_{B}), 67.4 (CH_{2Bn-6}), 65.6 (C-4_{A}), 65.4 (C-4_{B}), 55.3 (C-2_{B}), 49.5 (C-2_{A}), 23.2 (CH_{3Ac}), 17.2 (C-6_{B}). HRMS (ESI⁺): m/z [M+Na]⁺ calcd for C₃₃H₃₈Cl₃N₅O₁₀Na, 792.1582; found 792.1584.

AB Building Block from an A Donor Whereby the Protecting Groups at the 4_{A}-OH 6_{A}-OH are Orthogonal to Each Other

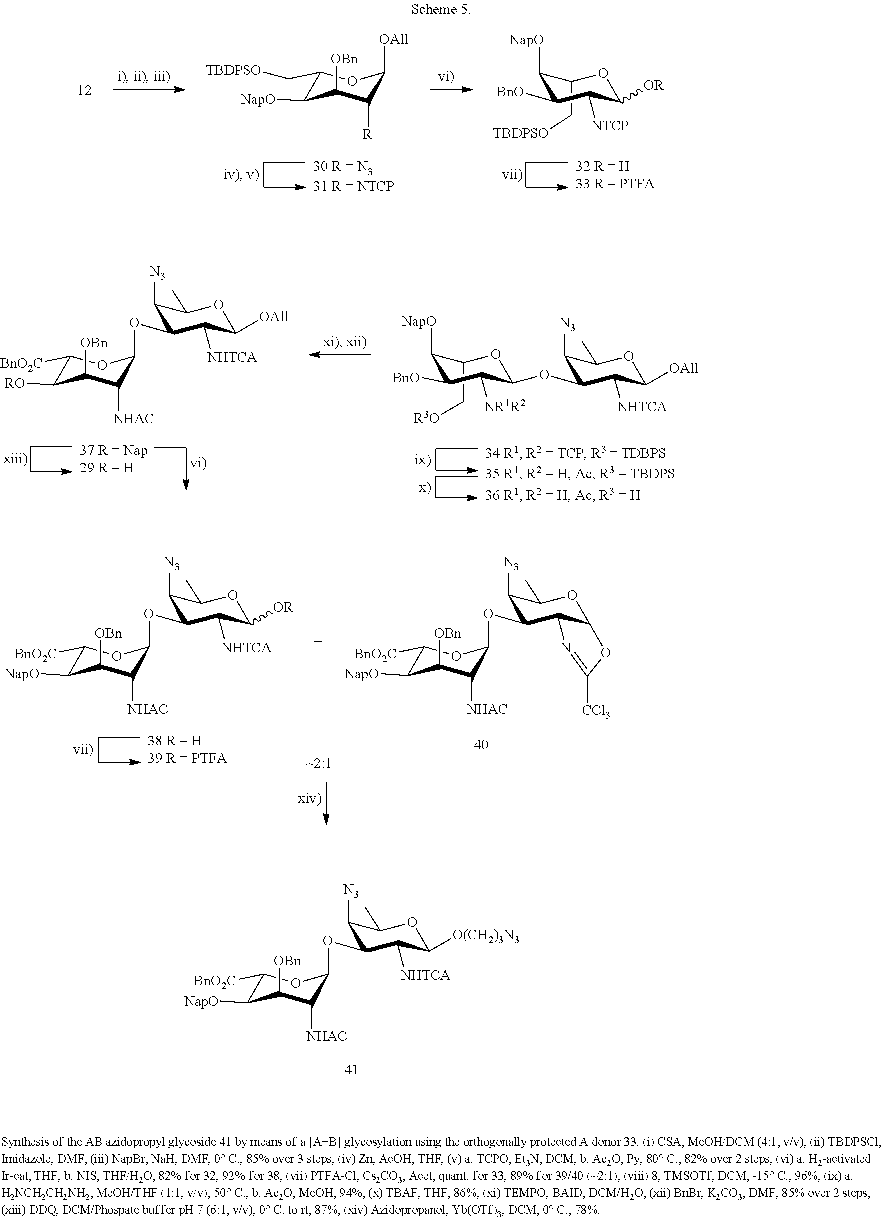

Allyl 2-azido-3-O-benzyl-6-O-tert-butyldiphenylsilyl-2-deoxy-4-O-(2-naphthylmethyl)-α-L-altropyranoside (30). CSA (4.1 g, 17.7 mmol, 0.5 equiv.) was added to acetal 12 (15.0 g, 35.4 mmol, 1.0 equiv.) in MeOH/DCM (4:1, 170 mL). After stirring at rt for 2 h, a TLC follow up (Tol/EtOAc 4:1) indicated reaction completion as shown by the absence of the starting 12 (R_{f} 0.65) and the presence of a non migrating spot. 5% Aq. NaHCO₃ (300 mL) was added followed by EtOAc (500 mL). The organic phase was separated and washed with brine (500 mL). The organic phase was dried over Na₂SO₄ and concentrated under reduced pressure. The crude product was dried under high vacuum. tert-Butyldiphenylchlorosilane (10.1 mL, 38.9 mmol, 1.1 equiv.) and imidazole (3.1 g, 46.0 mmol, 1.3 equiv.) were added to the crude diol in anhyd. DMF (180 mL) at 0° C. The reaction mixture was allowed to reach rt slowly and stirred overnight at this temperature. Methanol (10 mL) was added and after 30 min, volatiles were evaporated under reduced pressure. The residue was dissolved in EtOAc (500 mL) and the organic layer was washed with 90% aq. brine (500 mL), separated, dried over Na₂SO₄, and concentrated. 2-(Bromomethyl)naphthalene (10.9 g, 49.6 mmol, 1.4 equiv.) was added to the crude intermediate in DMF (230 mL). The solution was cooled to 0° C. and NaH (60% in mineral oil, 1.7 g, 70.8 mmol, 2.0 equiv.) was added portion wise. After stirring vigorously for 2 h while the bath temperature slowly reached rt, a TLC follow up indicated reaction completion. The reaction mixture was diluted with DCM (1 L) and 5% aq. NH₄Cl (500 mL) was added. The organic layer was washed with water (1.5 L) and brine (1 L), dried over Na₂SO₄ and concentrated. The crude product was purified by flash chromatography (cHex/EtOAc 12:1→10:1) to give the fully protected 30 (21.6 g, 30.2 mmol, 85%) as a light yellow oil. Allyl glycoside 30 had R_{f} 0.8 (Tol/EtOAc 10:1). ¹H NMR (CDCl₃) δ 7.84-7.30 (m, 22H, H_{Ar}), 5.98-5.88 (m, 1H, CH_{All}), 5.34-5.28 (m, 1H, CH_{2All}), 5.21-5.17 (m, 1H, CH_{2All}), 4.79 (d, 1H, J=12.2 Hz, CH_{2Nap}), 4.79 (d, 1H, CH_{2Nap}), 4.73 (d, 1H, J_{1,2}=4.7 Hz, H-1), 4.67 (d, 1H, J=11.9 Hz, CH_{2Bn}), 4.61 (d, 1H, CH_{2Bn}), 4.31-4.25 (m, 1H, CH_{2All}), 4.19 (pq, 1H, H-5), 4.07-4.01 (m, 1H, CH_{2All}), 3.98-3.94 3.97 (ddₚₒ, 1H, H-2), 3.95 (ddₚₒ, 1H, J_{4,5}=5.3 Hz, H-4), 3.77 (brd, 2H, J_{5,6a}=4.5 Hz, J_{5,6b}=4.5 Hz, H-6a, H-6b), 3.74 (ddₚₒ, 1H, J_{3,4}=3.5 Hz, J_{2,3}=8.0 Hz, H-3), 1.00 (s, 9H, CH_{3, TBDPS}). ¹³C NMR (CDCl₃), δ 137.8, 135.5, 133.2, 133.0 (C_{q, Ar}), 133.8 (CH_{All}), 135.6, 135.6, 129.7, 128.3, 128.1, 127.9, 127.7 (2C), 126.6 (2C), 126.0, 125.9, 125.8 (C_{Ar}), 117.2 (CH_{2All}), 98.7 (C-1_{A}, ¹J_{C,H}=170 Hz), 76.2 (C-3), 72.9 (C-5), 72.3 (CH₂Nap, CH_{2Bn}), 72.1 (C-4), 68.7 (CH_{2All}), 63.7 (C-6), 61.8 (C-2), 26.9, 26.7 (3C, CH_{3,TBDPS}), 19.1 (C_{TBDPS}). HRMS (ESI⁺): m/z [M+Na]⁺ calcd for C₃₄H₄₇N₃O₅SiNa, 736.3783; found 736.3177.

Allyl 3-O-benzyl-6-O-tert-butyldiphenylsilyl-2-deoxy-4-O-(2-naphthylmethyl)-2-tetrachlorophthalimido-α-L-altropyranoside (31). Zn dust (8.2 g, 126 mmol, 10.0 equiv.) and AcOH (7.2 mL, 126 mmol, 10.0 equiv.) were added to azide 30 (9.0 g, 12.6 mmol, 1.0 equiv.) in anhyd. THF (85 mL). After stirring for 1 h, a TLC analysis (Tol/EtOAc 10:1) showed the absence of azide 30 (R_{f} 0.8) and the presence of a more polar spot (R_{f} 0.0). The suspension was filtered over a pad of Celite and washed with DCM. The DCM layer was washed with satd aq. NaHCO₃, water, and brine, dried over Na₂SO₄, concentrated under reduced pressure, and dried under high vacuum. The crude amine was dissolved in DCM and tetrachlorophthalic anhydride (2.2 g, 7.5 mmol, 0.6 equiv.) was added. The mixture was stirred at rt for 30 min. Et₃N (2.1 mL, 15.1 mmol, 1.2 equiv.) was added followed by more tetrachlorophthalic anhydride (2.2 g, 7.5 mmol, 0.6 equiv.). The reaction was stirred for another 30 min at rt, at which time a TLC follow up (EtOAc) indicated reaction completion. Volatiles were evaporated and dried under high vacuum. The crude was dissolved in pyridine (60 mL) and Ac₂O (5.9 mL, 63.0 mmol, 5.0 equiv.) was added. After heating to 80° C. for 10 min, a TLC analysis (cHex/EtOAc 9:1) showed full consumption of the intermediate and the presence of a less polar spot. At completion, the mixture was concentrated under reduced pressure and coevaporated with toluene (30 mL) twice. The crude was taken in DCM (300 mL) and the DCM layer was washed with water (300 mL) and brine (300 mL), dried over Na₂SO₄, filtered, and concentrated under reduced pressure. The residue was purified by flash chromatography (cHex/EtOAc 93:7→88:12) to give the fully protected 31 (9.54 g, 10.4 mmol, 82%) as a yellowish dense oil. Allyl glycoside 31 had R_{f} 0.7 (cHex/EtOAc, 10:1). ¹H NMR (CDCl₃) δ 7.83-7.35 (m, 18H, H_{Ar}), 7.04 (brs, 4H, H_{Ar}), 5.83-5.73 (m, 1H, CH_{All}), 5.27 (d, 1H, J_{1,2}=7.0 Hz, H-1), 5.19-5.14 (m, 1H, CH_{2All}), 5.08-5.05 (m, 1H, CH_{2All}), 4.94 (d, 1H, J=12.7 Hz, CH_{2Nap}), 4.85 (dₚₒ, 1H, CH_{2Nap}), 4.85 (ddₚₒ, 1H, J_{2,3}=11.2 Hz, H-2), 4.60 (d, 1H, J=12.3 Hz, CH_{2Bn}), 4.3.6 (dd, 1H, J_{3,4}=3.6 Hz, H-3), 4.30 (dddₚₒ, 1H, J_{4,5}=3.3 Hz, H-5), 4.25-4.19 (m, 1H, CH_{2All}), 4.16 (pt, 1H, H-4), 4.10 (d, 1H, CH_{2Bn}), 3.98-3.93 (m, 1H, CH_{2All}), 3.83 (brd, 2H, J_{5,6a}=6.1 Hz, J_{5,6b}=6.1 Hz, H-6a, H-6b), 1.04 (s, 9H, CH_{3,TBDPS}). ¹³C NMR (CDCl₃) δ 163.2 (CO_{NTCP}), 157.4, 142.4, 139.7, 138.0, 135.9, 133.2, 133.1, 133.0 (2C), 129.5, 127.7, 127.2 (C_{q,Ar}), 133.9 (CH_{All}), 135.6 (2C), 129.8, 128.0, 127.9, 127.8, 127.6, 127.5, 127.3, 126.5, 126.0 (2C), 125.8 (C_{Ar}), 117.1 (CH_{2All}), 96.1 (C-1_{A}, ¹J_{C,H}=169 Hz), 75.6 (C-5), 74.3 (C-3), 72.7 (CH_{2Nap}), 72.1 (CH_{2Bn}), 71.8 (C-4), 68.6 (CH_{2All}), 63.1 (C-6), 53.4 (C-2), 26.9, 26.7 (3C, CH_{3,TBDPS}), 19.2 (C_{TBDPS}). HRMS (ESI⁺): m/z [M+NH₄]⁺ calcd for C₅₁H₅₁Cl₄N₂O₇Si, 971.2220; found 971.2213.

3-O-Benzyl-6-O-tert-butyldiphenylsilyl-2-deoxy-4-O-(2-naphthylmethyl)-2-tetrachlorophthalimido-α/β-L-altropyranose (32). [Ir(COD)(PMePh₂)₂]PF₆ (115 mg, 0.13 mmol, 0.02 equiv.) was dissolved in anhyd. THF (8.0 mL) and stirred for 30 min under an H₂ atmosphere. The resulting yellow solution was degassed repeatedly with Ar and transferred by means of a cannula into a solution of allyl glycoside 31 (6.5 g, 6.8 mmol, 1.0 equiv.) in anhyd. THF (60 mL). The reaction mixture was stirred for 1 h at rt, at which time a solution of NIS (1.68 g, 7.5 mmol, 1.1 equiv.) in H₂O (15 mL) was added. After stirring for 1 h at rt, a TLC analysis (cHex/EtOAc 8:1) revealed the full consumption of the isomerization product (R_{f} 0.65) and the presence of a more polar spot (R_{f} 0.1). 10% Aq. Na₂SO₃ was added and volatiles were evaporated. The aq. phase was extracted with DCM (200 mL) twice. The organic layers were combined, washed with water and brine, dried over Na₂SO₄, filtered, and concentrated under vacuum. Purification of the residue by flash chromatography (Tol/EtOAc 10:1→8:1) yielded the expected hemiacetal 32 (5.2 g, 5.6 mmol, 82%) as a white floppy solid. Hemiacetal 32 ((a/P 5:1) had R_{f} 0.6 (cHex/EtOAc 9:1). The ca anomer had ¹H NMR (CDCl₃) δ 7.84-6.99 (m, 22H, HM), 5.25 (dd, 1H, J_{1,OH}=9.4 Hz, J_{1,2}=6.8 Hz, H-1), 4.81 (d, 1H, J=12.4 Hz, CH_{2Nap}), 4.76 (d, 1H, CH_{2Nap}), 4.67 (dd, 1H, J_{2,3}=10.8 Hz, H-2), 4.53 (d, 1H, J=12.4 Hz, CH_{2Bn}), 4.43-4.33 (dd, 1H, J_{3,4}=3.0 Hz, H-3), 4.30 (ddd, J_{4,5}=1.8 Hz, 1H, H-5), 4.19 (dd, 1H, H-4), 4.03 (d, 1H, CH_{2Bn}), 3.91 (dd, 1H, J_{5,6a}=5.6 Hz, J_{6a,6b}=10.6 Hz, H-6a), 3.86 (dd, 1H, J_{6a,6b}=8.2 Hz, H-6b), 3.06 (d, 1H, OH), 1.03 (s, 9H, CH_{3TBDPS}). ¹³C NMR (CDCl₃) δ 163.2 (CO_{NTCP}), 139.8, 137.8, 137.9, 137.8, 135.6, 135.5, 135.4, 133.2, 133.1, 133.0, 132.8 (C_{q,Ar}), 135.6, 135.5, 129.9 (2C), 129.0, 128.2 (2C), 128.1 (2C), 127.9, 127.8 (2C), 127.7, 127.4, 126.8, 126.6, 126.0 (2C), 125.9, 125.2 (C_{q,Ar}), 91.5 (C-1_{A}, ¹J_{C,H}=171 Hz), 76.1 (C-3), 73.3 (C-5), 72.7 (CH_{2Nap}), 71.7 (CH_{2Bn}), 71.7 (C-4), 62.2 (C-6), 56.1 (C-2), 26.8 (CH_{3TBDPS}), 21.4 (C_{TBDPS}). HRMS (ESI⁺): m/z [M+NH₄]⁺ calcd for C₄₈H₄₇C₁₄N₂O₇Si, 931.1907; found 931.1880.

The β anomer had ¹H NMR (CDCl₃) δ 7.84-6.99 (m, 22H, H_{Ar}), 5.37 (dd, 1H, J_{1,2}=4.1 Hz, J_{1,OH}=5.6 Hz, H-1), 4.95 (dd, 1H, J_{2,3}=10.8 Hz, H-2), 4.95 (ddₚₒ, 1H, J_{3,4}=2.7 Hz, H-3), 4.90 (dₚₒ, 1H, CH_{2Nap}), 4.85 (d, 1H, J=12.6 Hz, CH_{2Nap}), 4.41 (d, 1H, J=11.6 Hz, CH_{2Bn}), 4.28 (bsₒ, 1H, H-4), 4.28-4.25 (m, 1H, H-5), 4.21 (dₚₒ, 1H, CH_{2Bn}), 3.99 (dₚₒ, 1H, H-6a), 3.93-3.84 (mₒ, 1H, H-6b), 3.48 (brs, 1H, OH), 1.08 (s, 9H, CH_{3TBDPS}). ¹³C NMR (CDCl₃) δ 163.9 (CO_{NTCP}), 139.8-125.3 (C_{Ar}), 92.6 (C-1_{A}, ¹J_{C,H}=175 Hz), 77.4 (C-3), 72.2 (CH_{2Nap}), 72.4 (C-4), 71.4 (CH_{2Bn}), 64.6 (C-6), 53.6 (C-2), 26.8 (CH_{3TBDPS}), 21.4 (C_{TBDPS}). HRMS (ESI⁺): m/z [M+NH₄]⁺ calcd for C₄₈H₄₇C₁₄N₂O₇Si, 931.1907; found 931.1880.

Allyl 3-O-benzyl-6-O-tert-butyldiphenylsilyl-2-deoxy-4-O-(2-napthylmethyl)-2-tetrachlorophthalimido-α-L-altropyranosyl-(1→3)-4-azido-2,4,6-trideoxy-2-trichloroacetamido-β-D-galactopyranoside (34). PTFACl (1.47 mL, 7.1 mmol, 1.3 equiv.) and Cs₂CO₃ (1.9 g, 6.0 mmol, 1.1 equiv.) were added to hemiacetal 32 (5.0 g, 5.4 mmol, 1.0 equiv.) in acetone (40 mL). After stirring for 2 h at rt, the reaction mixture was filtered through a pad of Celite and washed with DCM (50 mL) twice. The filtrate was concentrated under reduced pressure and dried under vacuum to give the crude donor 33 (6.0 g, 5.4 mmol, quant.), which was used as such in the next step. The PTFA donor 33 had R_{f} 0.85 (Tol/EtOAc 10:1). HRMS (ESI⁺): m/z [M+Na]⁺ calcd for C₅₆H₄₇C₁₄F₃N₂O₇SiNa, 1107.1757; found 1107.1755.

A mix of the crude PTFA donor 33 (6.0 g, 5.4 mmol, 1.1 equiv. theo.) and acceptor 8 (1.83 g, 4.9 mmol, 1.0 equiv.) were co-evaporated with anhyd. toluene (30 mL) and then dried under vacuum. Freshly activated MS 4 Å (4.0 g) was added to the starting materials in anhyd. DCM (90 mL) and the suspension was stirred for 1 h under an Ar atmosphere at rt. After cooling to −15° C., TMSOTf (49 μL, 0.05 equiv.) was added slowly and stirring went on for 40 min during which the bath temperature kept at −15° C. A TLC analysis (Tol/EtOAc 10:1) showed the absence of donor 33 and the presence of a new spot (R_{f} 0.5) in addition to a slight amount of hemiacetal 32 (R_{f} 0.4). At completion, Et₃N (80 μL) was added. The suspension was filtered through a fitted funnel and washed with DCM (50 mL) twice. Volatiles were evaporated and the residue was purified by flash chromatography (cHex/EtOAc 10:1→8:1) to give disaccharide 34 as a white solid (6.0 g, 4.7 mmol, 96%). The coupling product 34 had ¹H NMR (CDCl₃) δ 7.85-7.81 (m, 4H, H_{Ar}), 7.67-7.63 (m, 3H, H_{Ar}), 7.53-7.16 (m, 10H, H_{Ar}), 7.04-6.98 (m, 5H, H_{Ar}), 6.67 (m, 1H, J_{2,NH}=6.8 Hz, NH_{B}), 5.85-5.75 (m, 1H, CH_{All}), 5.43 (d, 1H, J_{1,2}=7.2 Hz, H-1_{A}), 5.23-5.17 (m, 1H, CH_{2All}), 5.14-5.11 (m, 1H, CH_{2All}), 4.94 (d, 1H, J=12.4 Hz, CH_{2Nap}), 4.87 (ddₚₒ, 1H, J_{2,3}=11.1 Hz, H-2_{A}), 4.81 (d, 1H, CH_{2Nap}), 4.73 (d, 1H, J_{1,2}=8.4 Hz, H-1_{B}), 4.61 (d, 1H, J=12.0 Hz, CH_{2Bn}), 4.50 (dd, 2H, J_{3,4}=3.5 Hz, J_{2,3}=10.7 Hz, H-3_{B}), 4.39 (dddₚₒ, 1H, J_{4,5}=3.3 Hz, H-5_{A}), 4.33 (ddₚₒ, 1H, J_{3,4}=3.5 Hz, H-3_{A}), 4.29-4.24 (m, 1H, CH_{2All}), 4.11 (ptₚₒ, 1H, H-4_{A}), 4.09 (dₚₒ, 1H, CH_{2Bn}), 4.00-3.94 (m, 1H, CH_{2All}), 3.86 (dₚₒ, 1H, J_{3,4}=3.8 Hz, H-4_{B}), 3.85 (dₚₒ, 1H, J_{5,6a}=6.4 Hz, H-6a_{A}), 3.79 (dd, 1H, J_{5,6b}=5.8 Hz, J_{6a,6b}=11.0 Hz, H-6b_{A}), 3.51 (dd, 1H, H-2_{B}), 3.47 (dq, J_{4,5}=1.3 Hz, H-5_{B}), 1.82 (d, 3H, J_{5,6}=6.4 Hz, H-6_{B}), 1.05 (s, 9H, CH_{3TBDPS}). ¹³C NMR (CDCl₃) δ 163.1 (CO_{NHTCA}), 161.5 (CO_{NTCP}), 139.8, 137.8, 137.7, 135.7, 133.2, 133.0 (2C), 132.9, 129.6, 127.5 (C_{q, Ar}), 133.5 (CH_{All}), 135.6, 135.2, 129.9, 129.0, 128.2, 128.1, 128.0, 127.9, 127.8, 127.7, 127.6, 127.4, 126.5, 126.0, 125.9, 125.2 (C_{Ar}), 117.8 (CH_{2All}), 98.5 (C-1_{A}, ¹J_{C,H}=171 Hz), 97.6 (C-1_{B}, ¹J_{C,H}=163 Hz), 92.2 (CCl₃), 75.9 (C-5_{A}), 75.5 (C-3_{B}), 73.7 (C-3_{A}), 72.6 (CH_{2Nap}), 72.1 (CH_{2Bn}), 71.4 (C-4_{A}), 70.0 (CH_{2All}), 69.2 (C-5_{B}), 65.4 (C-4_{B}), 63.1 (C-6_{A}), 53.3 (C-2_{B}), 53.1 (C-2_{A}), 26.9 (CH_{3TBDPS}), 19.3 (C_{TBDPS}), 17.2 (C-6_{B}). HRMS (ESI⁺): m/z [M+NH₄]⁺ calcd for C₅₉H₆OCl₇N₆O₁₀Si, 1285.1960; found 1285.1948.

Allyl 2-acetamido-3-O-benzyl-6-O-tert-butyldiphenylsilyl-2-deoxy-4-O-(2-naphthylmethyl)-α-L-altropyranosyl-(1→3)-4-azido-2,4,6-trideoxy-2-trichloroacetamido-β-D-galactopyranoside (35). Ethylenediamine (1.3 mL, 19.3 mmol, 4.0 equiv.) was added to disaccharide 34 (6.2 g, 4.8 mmol, 1.0 equiv.) in THF/MeOH (1:1, 100 mL) at rt and the reaction mixture was stirred at 50° C. for 72 h under an Ar atmosphere. A TLC analysis (Tol/EtOAc 7:3) revealed the absence of the starting 34 (R_{f} 1.0) and the presence of a new spot (R_{f} 0.55). The mixture was allowed to reach rt and Et₃N (2.0 mL) was added, followed by acetic anhydride (4.6 mL, 48.9 mmol, 10.0 equiv.). After stirring for 3 h at rt, a TLC analysis (Tol/EtOAc 7:3) showed the presence of a new spot (R_{f} 0.65) whereas the intermediate amine had been fully consumed. The suspension was filtered by passing through a pad of Celite, washed with DCM (15 mL) thrice and the filtrate was concentrated under reduced pressure. The residue was purified by column chromatography (cHex/EtOAc 10:1→7:1). Acetamide 35 was obtained as a white solid (4.8 g, 4.6 mmol, 94%). Disaccharide 35 had ¹H NMR (CDCl₃) δ 7.84-7.81 (m, 1H, H_{Ar}), 7.75-7.62 (m, 7H, H_{Ar}), 7.50-7.26 (m, 15H, H_{Ar}), 6.67 (d, 1H, J_{2,NH}=7.2 Hz, NH_{B}), 5.90-5.80 (m, 1H, CH_{All}), 5.28-5.23 (mₚₒ, 1H, CH_{2All}), 5.23 (dₚₒ, 1H, J_{2,NH}=8.8 Hz, NH_{A}), 5.19-5.16 (m, 1H, CH_{2All}), 4.89 (d, 1H, J_{1,2}=8.0 Hz, H-1_{B}), 4.78 (d, 1H, J=12.7 Hz, CH_{2Nap}), 4.74 (d, 1H, CH_{2Nap}), 4.71 (dₚₒ, 1H, CH_{2Bn}), 4.69 (brsₚₒ, 1H, J_{1,2}=1.8 Hz, H-1_{A}), 4.52 (d, 1H, J=12.1 Hz, CH_{2Bn}), 4.48 (ddₚₒ, 1H, J_{3,4}=3.7 Hz, J_{2,3}=10.9 Hz, H-3_{B}), 4.47-4.43 (mₒ, 2H, H-5_{A}), 4.41 (ddd, J_{2,3}=4.3 Hz, H-2_{A}), 4.34-4.29 (m, 1H, CH_{2All}), 4.06-4.01 (mₚₒ, 1H, CH_{2All}), 4.00 (ddₚₒ, 1H, J_{5,6a}=2.6 Hz, J_{6a,6b}=11.2 Hz, H-6a_{A}), 3.95 (ddₚₒ, 1H, J_{5,6a}=2.6 Hz, J_{6a,6b}=11.1 Hz, H-6b_{A}), 3.93 (ptₚₒ, 1H, H-3_{A}), 3.63 (dd, 1H, J_{3,4}=3.0 Hz, J_{4,5}=8.9 Hz, H-4_{A}), 3.57 (brd, 1H, H-4_{B}), 3.53 (brq, 1H, H-5_{B}), 3.41 (ddd, 1H, H-2_{B}), 1.74 (s, 3H, CH_{3NHAc}), 1.17 (d, 3H, J_{5,6}=6.3 Hz, H-6_{B}), 1.08 (s, 9H, CH_{3TBDPS}). ¹³C NMR (CDCl₃) δ 168.8 (CO_{NHTA}), 162.1 (CO_{NHAc}), 138.6, 135.1, 133.5, 133.0 (2C, C_{q,Ar}), 133.6 (CH_{All}), 135.7, 135.6, 129.7 (2C), 128.2 (2C), 127.8, 127.7, 127.6, 127.5, 126.8, 126.1, 125.9 (2C, C_{Ar}), 117.7 (CH_{2All}), 101.5 (C-1_{A}, ¹J_{C,H}=168 Hz), 97.5 (C-1_{B}, ¹J_{C,H}=163 Hz), 92.2 (CCl₃), 76.0 (C-3_{B}), 72.5 (C-3_{A}), 71.5 (CH_{2Nap}), 70.5 (CH_{2Bn}), 70.4 (C-4_{A}), 70.1 (CH_{2All}), 69.7 (C-5_{A}), 69.7 (C-5_{B}), 65.2 (C-4_{B}), 63.7 (C-6_{A}), 55.8 (C-2_{B}), 49.6 (C-2_{A}), 27.0 (CH_{3TBDPS}), 23.0 (CH_{3NHAc}), 19.4 (C_{TBDPS}), 17.1 (C-6_{B}). HRMS (ESI⁺): m/z [M+H]⁺ calcd for C₅₃H₆₁Cl₃N₅O₉Si, 1044.3304; found 1044.3325.

Allyl 2-acetamido-3-O-benzyl-2-deoxy-4-O-(2-naphthylmethyl)-α-L-altropyranosyl-(1→3)-4-azido-2,4,6-trideoxy-2-trichloroacetamido-β-D-galactopyranoside (36). TBAF (1.8 g, 5.8 mmol, 1.2 equiv.) was added to disaccharide 35 (4.8 g, 4.8 mmol, 1.0 equiv.) in THF (98 mL) and the reaction mixture was stirred at rt for 4 h. A TLC analysis (Tol/EtOAc 7:3) showed the consumption of the fully protected 35 (R_{f} 0.65) and the presence of a polar spot. Acetic acid (0.34 mL, 5.8 mmol, 1.2 equiv.) was added and after stirring for 10 min, volatiles were evaporated. The residue was purified by flash chromatography (EtOAc/MeOH 100:0→95:5) to give alcohol 33 (3.2 g, 3.9 mmol, 86%) as a white solid. Disaccharide 36 had R_{f} 0.15 (EtOAc). ¹H NMR (DMSO-d₆) δ 8.87 (d, 1H, J_{2,NH}=9.2 Hz, NH_{B}), 7.93-7.82 (m, 4H, NH_{A}, H_{Ar}), 7.75 (brs, 1H, H_{Ar}), 7.53-7.47 (m, 2H, H_{Ar}), 7.43-7.39 (m, 3H, H_{Ar}), 7.32-7.24 (m, 3H, H_{Ar}), 5.85-5.76 (m, 1H, CH_{All}), 5.26-5.20 (m, 1H, CH_{2All}), 5.12-5.09 (m, 1H, CH_{2All}), 4.81 (d, 1H, J_{1,2}=1.6 Hz, H-1_{A}), 4.69 (dₚₒ, 3H, J=11.6 Hz, CH_{2Nap}), 4.67-4.62 (m, 2H, J=11.8 Hz, OH, CH_{2Bn}), 4.53 (dₚₒ, 1H, CH_{2Bn}), 4.51 (dₚₒ, 1H, J_{1,2}=8.9 Hz, H-1_{B}), 4.49 (dₚₒ, 1H, CH_{2Nap}), 4.32 (ddd, 1H, J_{2,NH}=8.4 Hz, J_{2,3}=4.5 Hz, H-2_{A}), 4.24-4.16 (m, 2H, H-5_{A}, CH_{2All}), 4.12 (ddₚₒ, 1H, J_{3,4}=3.6 Hz, J_{2,3}=10.8 Hz, H-3_{B}), 4.05 (brd, 1H, H-4_{B}), 4.00-3.95 (m, 1H, CH_{2All}), 3.90 (dddₚₒ, 1H, H-2_{B}), 3.79 (ddₚₒ, 1H, H-3_{A}), 3.78-3.74 (mₒ, 1H, H-6a_{A}), 3.72 (bq, 1H, H-5_{B}), 3.68 (dd, 1H, J_{3,4}=3.1 Hz, J_{4,5}=8.9 Hz, H-4_{A}), 3.53 (ddd, 1H, H-6b_{B}), 1.74 (s, 3H, CH_{3NHAc}), 1.24 (s, 9H, CH_{3TBDPS}). ¹³C NMR (DMSO-d₆) δ 169.1 (CO_{NHTCA}), 162.0 (CO_{NHAc}), 139.3, 136.4, 133.1, 132.9 (C_{q,Ar}), 134.8 (CH_{All}), 128.3, 128.1 (2C), 128.0, 127.6, 126.5, 126.4, 126.3 (C_{Ar}), 116.8 (CH_{2All}), 101.9 (C-1_{A}, ¹J_{C,H}=171 Hz), 100.2 (C-1_{B}, ¹J_{C,H}=162 Hz), 93.5 (CCl₃), 77.5 (C-3_{B}), 73.8 (C-3_{A}), 72.5 (C-4_{A}), 70.9 (CH_{2Nap}), 70.4 (CH_{2Bn}), 70.2 (C-5_{B}), 69.6 (C-5_{A}), 69.3 (CH_{2All}), 65.3 (C-4_{B}), 61.6 (C-6_{A}), 53.4 (C-2_{B}), 49.4 (C-2_{A}), 22.9 (CH_{3NHAc}), 17.6 (C-6_{B}). HRMS (ESI⁺): m/z [M+H]⁺ calcd for C₃₇H₄₃Cl₃N₅O₉, 806.2126; found 806.2117.

Allyl (benzyl 2-acetamido-3-O-benzyl-2-deoxy-4-O-(2-naphthylmethyl)-α-L-altropyranosyluronate)-(1→3)-4-azido-2,4,6-trideoxy-2-trichloroacetamido-β-D-galactopyranoside (37). TEMPO (116 mg, 0.74 mmol, 0.2 equiv.) was added, followed by BAIB (3.0 g, 9.3 mmol, 2.5 equiv.), to a suspension of alcohol 36 (3.0 g, 3.7 mmol, 1.0 equiv.) in DCM/H₂O (2:1, 120 mL). The biphasic mixture stirred vigorously for 2 h at rt, at which point a TLC analysis (EtOAc) revealed the absence of alcohol 36 (R_{f} 0.15) and the presence of a polar product (R_{f} 0.0). 10% Aq. Na₂SO₃ was added followed by DCM (80 mL). The DCM layer was separated, and the aq. phase was extracted with DCM (100 mL) twice. The combined organic phases were dried by passing through a phase separator filter and concentrated to dryness. The residue was dissolved in anhyd. DMF (40 mL). Benzyl bromide (1.3 mL, 11.1 mmol, 3.0 equiv.) and K₂CO₃ (670 mg, 4.8 mmol, 1.3 equiv.) were added and the suspension was stirred at rt for 2 h. At completion, satd aq. NH₄Cl was added and the aq. layer was washed with DCM (100 mL) thrice. The organic phases were combined, washed with brine (300 mL), dried over Na₂SO₄, filtered, and concentrated under reduced pressure. The residue was purified by flash chromatography (Tol/EtOAc 7:3→6:4) to give the desired benzyl ester 37 (2.8 g, 3.0 mmol, 85%) as a brown-white solid. Ester 37 had R_{f} 0.3 (Tol/EtOAc, 4:1). ¹H NMR (CDCl₃) δ 7.84-7.26 (m, 17H, H_{Ar}), 6.94 (d, J_{2,NH}=7.2 Hz, NH_{B}), 5.90-5.80 (m, 1H, CH_{All}), 5.73 (d, J_{2,NH}=6.8 Hz, NH_{A}), 5.33 (d, 1H, J_{1,2}=5.6 Hz, H-1_{A}), 5.24-5.20 (mₚₒ, 1H, CH_{2All}), 5.22 (dₚₒ, 1H, CH_{2Bn-6}), 5.19-5.15 (mₚₒ, 1H, CH_{2All}), 5.17 (d, 1H, J=12.0 Hz, CH_{2Bn-6}), 4.80 (d, 1H, J_{4,5}=4.6 Hz, H-5_{A}), 4.77 (d, 1H, J_{1,2}=8.3 Hz, H-1_{B}), 4.73 (d, 1H, J=12.7 Hz, CH_{2Nap}), 4.71 (d, 1H, CH_{2Nap}), 4.52 (d, 1H, J=12.0 Hz, CH_{2Bn}), 4.48 (ddₚₒ, 1H, J_{3,4}=3.8 Hz, H-3_{B}), 4.48 (d, 1H, CH_{2Bn}), 4.33-4.28 (m, 1H, CH_{2All}), 4.10 (ddₚₒ, J_{3,4}=2.7 Hz, H-4_{A}), 4.07-3.98 (m, 3H, H-3_{A}, H-2_{A}, CH_{2All}), 3.93 (brd, 1H, J_{3,4}=3.2 Hz, H-4_{B}), 3.57 (dt, 1H, J_{2,3}=10.5 Hz, H-2_{B}), 3.47 (brq, 1H, H-5_{B}), 1.86 (s, 3H, CH_{3Ac}), 1.23 (d, 3H, J_{5,6}=6.3 Hz, H-6_{B}). ¹³C NMR (CDCl₃) δ 170.4 (CO_{NTCA}), 169.3 (C-6_{A}), 161.9 (CO_{NAc}), 137.9, 134.9, 134.8, 133.1, 133.0 (C_{q, Ar}), 133.5 (CH_{All}), 128.7 (2C), 128.6, 128.3, 128.1, 127.9, 127.8, 127.6, 126.9, 126.1, 126.0, 125.9 (C_{Ar}), 117.9 (CH_{2All}), 99.5 (C-1_{A}, ¹J_{C,H}=169 Hz), 97.6 (C-1_{B}, ¹J_{C,H}=162 Hz), 92.4 (CCl₃), 76.6 (C-3_{B}), 73.4 (C-3_{A}), 72.9 (C-4_{A}), 71.9 (C-5_{A}), 71.8 (2C, CH_{2Bn}, CH_{2Nap}), 70.1 (CH_{2All}), 69.3 (C-5_{B}), 67.5 (CH_{2Bn-6}), 65.1 (C-4_{B}), 55.0 (C-2_{B}), 52.1 (C-2_{A}), 23.4 (CH_{3Ac}), 17.3 (C-6_{B}). HRMS (ESI⁺): m/z [M+H]⁺ calcd for C₄₄H₄₇Cl₃N₅O₁₀, 910.2388; found 910.2380.

Allyl (benzyl 2-acetamido-3-O-benzyl-2-deoxy-α-L-altropyranosyluronate)-(1→3)-4-azido-2,4,6-trideoxy-2-trichloroacetamido-β-D-galactopyranoside (29). The benzyl ester 37 (see below, 340 mg, 374 μmol, 1.0 equiv.) was dissolved in DCM (6.0 mL) and phosphate buffer pH 7 (1.0 mL) was added. The biphasic mixture was cooled to 0° C. and DDQ (144 mg, 636 μmol, 1.7 equiv.) was added. Stirring was pursued for 2 h while the bath was allowed to reach rt. The mixture was diluted with DCM (10 mL) and the DCM layer was washed with satd aq. NaHCO₃, water and brine, then dried over Na₂SO₄, and concentrated under reduced pressure. The residue was purified by flash chromatography (Tol/EtOAc 65:35→55:45) to give alcohol 29 (290 mg, 325 μmol, 87%) as a white solid. Disaccharide 29 had R_{f} 0.3 (Tol/EtOAc 1:1). ¹H NMR (CDCl₃) δ 7.43-7.31 (m, 10H, H_{Ar}), 6.81 (d, 1H, J_{2,NH}=7.2 Hz, NH_{B}), 5.89-5.79 (m, 1H, CH_{All}), 7.93 (d, 1H, J_{2,NH}=8.4 Hz, NH_{A}), 5.28 (d, 1H, J_{2,NH}=12.1 Hz, CH_{2Bn-6}, CH_{2All}), 5.29-5.23 (mₚₒ, 1H, CH_{2All}), 5.22 (dₚₒ, 1H, CH_{2Bn-6}), 5.20-5.16 (m, 1H, CH_{2All}), 4.93 (d, 1H, J_{1,2}=3.2 Hz, H-1_{A}), 4.78 (dₚₒ, 1H, J_{1,2}=8.4 Hz, H-1_{B}), 4.73 (d, 1H, J=11.8 Hz, CH_{2Bn}), 4.69 (dₚₒ, 1H, J_{4,5}=7.8 Hz, H-5_{A}), 4.66 (d, 1H, CH_{2Bn}), 4.51 (dd, 1H, J_{3,4}=3.7 Hz, J_{2,3}=10.7 Hz, H-3_{B}), 4.34-4.39 (m, 2H, H-2_{A}, CH_{2All}), 4.12 (ddd, 1H, J_{4,5}=7.8 Hz, H-4_{A}), 4.07-4.01 (m, 1H, CH_{2All}), 3.85 (dd, 1H, J_{2,3}=5.0 Hz, J_{3,4}=3.5 Hz, H-3_{A}), 3.80 (d, 1H, J_{3,4}=3.2 Hz, H-4_{B}), 3.61 (dq, 1H, J_{4,5}=0.9 Hz, H-5_{B}), 3.55 (ddd, J_{2,3} 10.8 Hz, 1H, H-2_{B}), 2.76 (d, 1H, J_{4,OH}=8.2 Hz, OH), 1.95 (s, 3H, CH_{3Ac}), 1.27 (d, 3H, J_{5,6}=6.4 Hz, H-6_{B}). ¹³C NMR (CDCl₃) δ 169.5 (CO_{NHTCA}), 169.4 (C-6), 162.1 (CO_{NHAc}), 137.6, 135.0 (C_{q,Ar}), 133.5 (CH_{All}), 128.7, 128.6 (2C), 128.4 (2C), 128.2, 128.0 (C_{Ar}), 117.9 (CH_{2All}), 100.6 (C-1_{A}, ¹J_{C,H}=170 Hz), 97.6 (C-1_{B}, ¹J_{C,H}=162 Hz), 92.2 (CCl₃), 76.7 (C-3_{B}), 75.2 (C-3_{A}), 72.0 (CH_{2Bn}), 71.0 (C-5_{A}), 70.0 (CH_{2All}), 69.6 (C-5_{B}), 67.4 (CH_{2Bn-6}), 65.6 (C-4_{A}), 65.4 (C-4_{B}), 55.3 (C-2_{B}), 49.5 (C-2_{A}), 23.2 (CH_{3Ac}), 17.2 (C-6_{B}). HRMS (ESI⁺): m/z [M+Na]⁺ calcd for C₃₃H₃₈Cl₃N₅O₁₀Na, 792.1582; found 792.1584.

(Benzyl 2-acetamido-3-O-benzyl-2-deoxy-4-O-(2-naphthylmethyl)-α-L-altropyranosyluronate)-(1→3)-4-azido-2,4,6-trideoxy-2-trichloroacetamido-α/β-D-galactopyranose (38). [Ir(COD)(PMePh₂)₂]PF₆ (13 mg, 15 μmol, 0.02 equiv.) in anhyd. THF (4.0 mL) was degassed repeatedly and stirred for 30 min under a hydrogen atmosphere. The resulting yellow solution was degassed several times with Ar and transferred by means of a cannula into a solution of allyl glycoside 37 (700 mg, 770 μmol, 1.0 equiv.) in anhyd. THF (10 mL). After stirring for 2 h at rt, NIS (191 mg, 847 μmol, 1.1 equiv.) and H₂O (12 mL) were added. After stirring for an additional hour, a TLC analysis (Tol/EtOAc 7:3) showed the complete consumption of disaccharide 37 (R_{f} 0.45) and the presence of a polar spot (R_{f} 0.2). 10% Aq. Na₂SO₃ was added. Volatiles were removed under reduced pressure and the aq. phase was extracted with DCM (20 mL) twice. The combined organic layers were washed with brine, dried over anhyd. Na₂SO₄, and concentrated. Purification of the residue by flash chromatography (cHex/EtOAc 5:1→4:1) gave the expected hemiacetal 38 (620 mg, 713 μmol, 92%) as a white solid. Hemiacetal 38, isolated as a 10:7 cc/p mixture had R_{f} 0.15 (Tol/EtOAc 1:1). The major anomer had ¹H NMR (CDCl₃) δ 7.84-7.26 (m, 17H, H_{Ar}), 6.94 (d, J_{2,NH}=7.2 Hz, NH_{B}), 5.90-5.80 (m, 1H, CH_{All}), 5.73 (d, J_{2,NH}=6.8 Hz, NH_{A}), 5.33 (d, 1H, J_{1,2}=5.6 Hz, H-1_{A}), 5.27-5.22 (m, 1H, CH_{2All}), 5.20-5.14 (mₚₒ, 3H, CH_{2All}, CH_{2Bn-6}), 4.80 (d, 1H, J_{4,5}=4.4 Hz, H-5_{A}), 4.77 (d, 1H, J_{1,2}=8.4 Hz, H-1_{B}), 4.70 (brs, 2H, CH_{2Nap}), 4.53-4.46 (m, 3H, H-3_{B}, CH_{2Bn}), 4.33-4.28 (m, 1H, CH_{2All}), 4.10 (ddₚₒ, J_{3,4}=2.8 Hz, H-4_{A}), 4.07-4.00 (m, 3H, H-3_{A}, H-2_{A}, CH_{2All}), 3.93 (brd, 1H, J_{3,4}=3.2 Hz, H-4_{B}), 3.57 (dddₚₒ, 1H, J_{2,3}=9.2 Hz, H-2_{B}), 3.47 (brq, 1H, H-5_{B}), 1.86 (s, 3H, CH₃NAc), 1.23 (d, 3H, J_{5,6}=6.4 Hz, H-6_{B}). ¹³C NMR (CDCl₃) δ 170.4 (CO_{NTCA}), 169.3 (C-6_{A}), 161.9 (CO_{NAc}), 137.9, 134.9, 134.8, 133.1, 133.0 (C_{q, Ar}), 133.5 (CH_{All}), 128.7 (2C), 128.6, 128.3, 128.1, 127.9, 127.8, 127.6, 126.9, 126.1, 126.0, 125.9 (C_{Ar}), 117.9 (CH_{2All}), 99.5 (C-1_{A}, ¹J_{C,H}=169 Hz), 97.6 (C-1_{B}, ¹J_{C,H}=162 Hz), 92.4 (CCl₃), 76.6 (C-3_{B}), 73.4 (C-3_{A}), 72.9 (C-4_{A}), 71.9 (C-5_{A}), 71.8 (2C, CH_{2Bn}, CH_{2Nap}), 70.1 (CH_{2All}), 69.3 (C-5_{B}), 67.5 (CH_{2Bn-6}), 65.1 (C-4_{B}), 55.0 (C-2_{B}), 52.1 (C-2_{A}), 23.4 (CH_{3Ac}), 17.3 (C-6_{B}). HRMS (ESI⁺): m/z [M+H]⁺ calcd for C₄₁H₄₃Cl₃N₅O₁₀, 870.2076; found 870.2070.

(Benzyl 2-acetamido-3-O-benzyl-2-deoxy-4-O-(2-naphthylmethyl)-α-L-altropyranosyluronate)-(1→3)-4-azido-2,4,6-trideoxy-2-trichloroacetamido-α/β-D-galactopyranosyl (N-phenyl)trifluoroacetimidate (39) and 2-Trichloromethyl-[(benzyl 2-acetamido-3-O-benzyl-2-deoxy-4-O-(2-naphthylmethyl)-α-L-altropyranosyluronate)-(1→3)-4-azido-1,2,4,6-tetradeoxy-α-D-galactopyrano]-[2,1,d]-oxazoline (40). Hemiacetal 38 (630 mg, 725 μmol, 1.0 equiv.) was dissolved in acetone (10 mL). PTFACl (149 μl, 942 mol, 1.3 equiv.) was added followed by Cs₂CO₃ (260 mg, 797 μmol, 1.1 equiv.). The reaction mixture was stirred for 2 h at rt under an Ar atmosphere. A TLC follow up (Tol/EtOAc 5:1) showed that the starting 38 had evolved into two less polar spots (R_{f} 0.3 and 0.35). The reaction mixture was filtered over a pad of Celite, washed with acetone (10 mL) twice and the filtrate was concentrated under reduced pressure. The residue was purified by rapid flash chromatography (cHex/EtOAc 4:1→2:1, 1% Et₃N) to give the desired donor as a 3:2 mix of 39 and 40 (670 mg, 644 μmol, 89%) isolated as a floppy white solid. The isolated mix of donors 39 and 40 had ¹H NMR (CDCl₃) δ 7.87-6.80 (m, 22H_{Ar,PTFA}, NH_{B,PTFA}, 17H-_{Ar,oxa}), 6.56 (bs, 0.6H, H-1_{B,PTFA}), 6.18 (d, 0.4H, J_{1,2}=7.0 Hz, H-1_{B,oxa}), 5.86 (d, 0.4H, J_{2,NH}=7.2 Hz, NH_{A,oxa}), 5.48 (d, 0.4H, J_{1,2}=5.6 Hz, H-1_{A,oxa}), 5.41 (dₚₒ, 0.6H, J_{1,2}=7.6 Hz, H-1_{A,PTFA}), 5.39 (dₚₒ, J_{1,2}=8.0 Hz, NH_{A,PTFA}), 5.23 (d, 0.4H, J=12 Hz, CH_{2Bn-6}), 5.16 (sₚₒ, 1.2H, CH_{2Bn-6}), 5.14 (dₚₒ, 0.4H, CH_{2Bn-6}), 4.87 (d, 0.6H, J=12.5 Hz, CH_{2Nap}), 4.81 (d, 0.6H, CH_{2Nap}), 4.78 (dₚₒ, 0.6H, J_{3,4}=3.0 Hz, H-5_{A}), 4.76 (dₚₒ, 0.6H, J_{3,4}=4.9 Hz, H-5_{A}), 4.69 (d, 0.4H, J=12.4 Hz, CH_{2Nap}), 4.64 (d, 0.4H, CH_{2Nap}), 4.59 (d, 0.4H, J=12.1 Hz, CH_{2Bn}), 4.54 (d, 0.4H, CH_{2Bn}), 4.48 (dd, 0.6H, J_{3,4}=3.1 Hz, J_{3,4}=11.0 Hz, H-3_{B}), 4.42 (do, 0.6H, J=12.0 Hz, CH_{2Bn}), 4.42-4.37 (mₒ, 0.6H, H-2_{B}), 4.30 (ddd, 0.6H, H-2_{A}), 4.26 (bs, 0.6H, H-4_{B}), 4.20-4.23 (m, 2.2H, CH_{2Bn}, H-3_{A}, H-4_{A}, H-2_{B}), 4.10 (ddₚₒ, 0.6H, H-4_{A}), 4.08-4.03 (mₚₒ, 0.4H, H-2_{A}), 4.05-4.99 (m, 0.6H, H-5_{B}), 3.86 (m, 0.4H, H-4_{B}), 3.75 (dd, 0.4H, H-5_{B}), 3.57 (dd, 0.4H, J_{3,4}=3.7 Hz, J_{2,3}=8.1 Hz, H-3_{B}), 3.54 (dd, 0.6H, J_{2,3}=10.1 Hz, J_{3,4}=2.8 Hz, H-3_{A}), 1.94 (s, 1.8H, CH_{3Ac}), 1.93 (s, 1.2H, CH_{3Ac}), 1.28 (dₚₒ, 0.6H, J_{5,6}=6.3 Hz, H-6_{B}), 1.27 (dₚₒ, 0.4H, J_{5,6}=6.3 Hz, H-6_{B}). ¹³C NMR (Partial, CDCl₃) δ 173.2, 170.7, 170.5 (CO_{NTCA}), 169.3, 168.9 (C-6_{A}), 162.9, 162.1 (CO_{NAc}), 143.0, 137.9, 137.3, 135.0, 134.9, 134.7, 134.6, 133.1, 133.0 (C_{q,Ar}), 129.1, 128.8 (2C), 128.6, 128.5, 128.4, 128.3 (2C), 128.2, 128.0 (2C), 127.9 (2C), 127.8, 127.6, 127.2, 126.9, 126.8, 126.2, 126.1 (2C), 126.0 (2C), 125.9, 124.4, 119.4, 118.3 (C_{Ar}), 107.6 (C-1_{B,oxa}, ¹J_{C,H}=183 Hz), 99.0 (C-1_{A}, ¹J_{C,H}=169 Hz), 98.8 (C-1_{A}, ¹J_{C,H}=171 Hz), 93.7 (bs, C-1_{B-PTFA}), 92.4 (CCl₃), 86.9 (CCl₃), 81.1 (C-3_{A}), 74.5 (C-3_{B}), 73.6 (C-5_{A}), 73.1 (C-4_{A}), 73.0 (C-4_{A}), 72.5 (C-3_{B}), 72.1 (CH_{2Nap}), 71.8 (2C, CH_{2Nap}, CH_{2Bn}), 71.5 (C-5_{A}), 71.4 (2C, C-3_{A}, CH_{2Bn}), 69.0 (C-5_{B}), 68.0 (C-5_{B}), 67.4 (2C, CH_{2Bn-6}), 64.2 (C-4_{B}), 63.5 (C-2_{B}), 61.3 (C-4_{B}), 52.3 (C-2_{A}), 51.0 (C-2_{A}), 50.3 (C-2_{B}), 23.7, 23.4 (CH_{3Ac}), 17.5, 17.3 (C-6_{B}). HRMS (ESI⁺): m/z [M+H]⁺ calcd for C₄₉H₄₇Cl₃F₃N₆O₁₀, 1041.2372; found 1041.2378.

3-Azidopropyl (benzyl 2-acetamido-3-O-benzyl-2-deoxy-4-O-(2-naphthylmethyl)-α-L-altropyranosyluronate)-(1→3)-4-azido-2,4,6-trideoxy-2-trichloroacetamido-β-D-galactopyranoside (31). Freshly activated MS 4 Å (50 mg) was added to donors 39/40 (2:1, 50 mg, 48 μmol, 1.0 equiv.) in anhyd. DCM (3.0 mL) containing 3-azidopropanol (22 μL, 240 μmol, 5.0 equiv.). The reaction mixture was stirred for 45 min at rt in an Ar atmosphere and cooled to 0° C. Yb(OTf)₃ (3.0 mg, 5.0 μmol, 0.1 equiv.) was added and after stirring for 30 min at 0° C., a TLC analysis (Tol/EtOAc 7:3) confirmed the absence of donors 39/40 (R_{f} 0.45, 0.55) and the presence of a major compound (R_{f} 0.2). Et₃N (2.0 μL) was added and the suspension was filtered over a fitted funnel, and washed thoroughly with DCM. The filtrate was concentrated under reduced pressure and the residue was purified by flash chromatography (Tol/EtOAc 60:40→50:50) to give the condensation product 41 (36 mg, 37 μmol, 78%) as a white solid. The azidopropyl glycoside 41 had R_{f} 0.2 (Tol/EtOAc 4:1). ¹H NMR (CDCl₃) δ 7.85-7.71 (m, 4H, H_{Ar}), 7.51-7.47 (m, 2H, H_{Ar}), 7.42-7.25 (m, 11H, H_{Ar}), 6.83 (d, J_{2,NH}=7.2 Hz, NH_{B}), 5.55 (d, J_{2,NH}=7.2 Hz, NH_{A}), 5.28 (d, 1H, J_{1,2}=5.9 Hz, H-1_{A}), 5.22 (d, H, J=12.0 Hz, CH_{2Bn-6}), 5.19 (d, H, CH_{2Bn-6}), 4.82 (d, 1H, J_{4,5}=4.8 Hz, H-5_{A}), 4.75 (d, H, J=12.5 Hz, CH_{2Nap}), 5.22 (d, H, CH_{2Nap}), 4.65 (d, 1H, J_{1,2}=8.4 Hz, H-1_{B}), 4.53 (d, 1H, J=11.9 Hz, CH_{2Bn}), 4.45 (d, 1H, CH_{2Bn}), 4.39 (dd, 1H, J_{3,4}=3.5 Hz, J_{2,3}=10.8 Hz, H-3_{B}), 4.11 (dd, J_{3,4}=2.8 Hz, H-4_{A}), 4.07 (pdt, 1H, H-2_{A}), 3.97 (ddₚₒ, 1H, J_{2,3}=8.2 Hz, H-3_{A}), 3.93-3.88 (m, 2H, H-4_{B}, OCH₂), 3.62 (ddd, 1H, H-2_{B}), 3.56-3.51 (m, 1H, OCH₂), 3.49 (brq, 1H, H-5_{B}), 3.38 (t, 2H, J=6.8 Hz, NCH₂), 1.89-1.78 (m, 2H, CH₂), 1.87 (s, 3H, CH_{3NHAc}), 1.23 (d, 3H, J_{5,6}=6.2 Hz, H-6_{B}). ¹³C NMR (CDCl₃) δ 170.3 (CO_{NHTCA}), 169.3 (C-6_{A}), 161.8 (CO_{NHAc}), 137.8, 134.9, 134.8, 133.1, 133.0 (C_{q,Ar}), 128.7, 128.6, 128.3, 128.2, 128.1, 127.8 (2C), 127.7, 126.8, 126.1, 126.0, 125.9 (C_{Ar}), 99.5 (C-1_{A}, ¹J_{C,H}=171 Hz), 97.2 (C-1_{B}, ¹J_{C,H}=162 Hz), 92.5 (CCl₃), 76.5 (C-3_{B}), 73.6 (C-3_{A}), 72.7 (C-4_{A}), 71.9 (C-5_{A}), 71.8 (2C, CH_{2Bn}, CH_{2Nap}), 69.4 (C-5_{B}), 67.4 (CH_{2Bn-6}), 66.3 (OCH₂), 64.9 (C-4_{B}), 54.7 (C-2_{B}), 52.0 (C-2_{A}), 48.1 (CH₂N₃), 29.0 (CH₂), 23.5 (CH_{3Ac}), 17.3 (C-6_{B}). HRMS (ESI⁺): m/z [M+H]⁺ calcd for C₄₄H₄₈Cl₃N₈O₁₀, 953.2559; found 953.2542.

### Example 2: Strategy 2_{A}-NAcBoc,2_{B}-NTCA, 4_{A}-Nap Series

Use of an Acid-Sensitive Acetamide Camouflage of the 2_{A}-NHAc

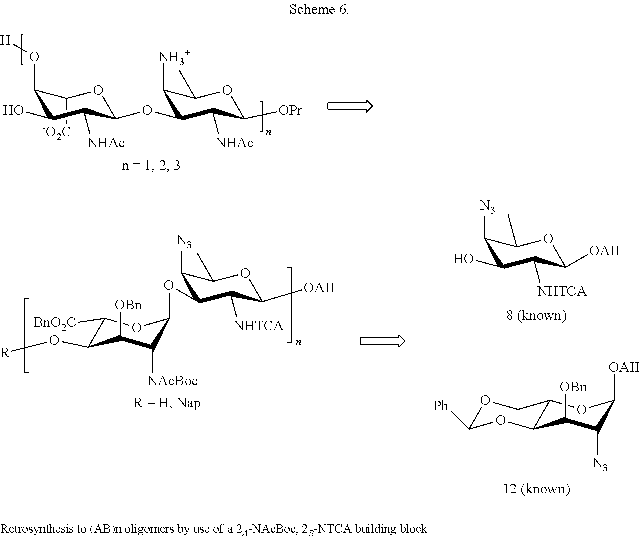

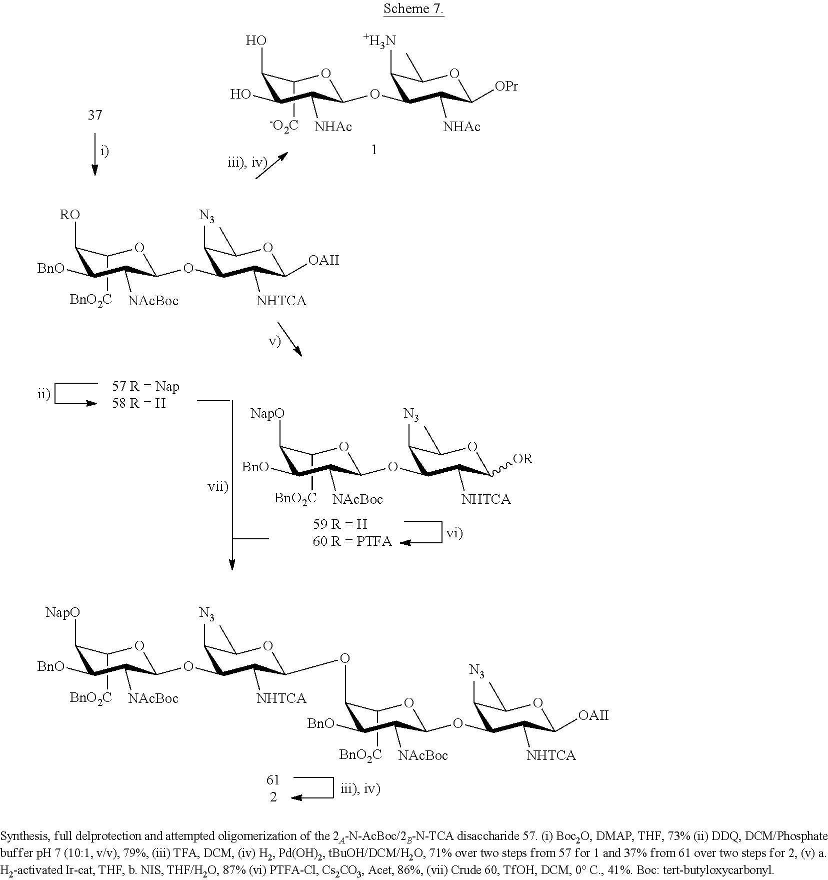

Allyl (benzyl 2-(N-tert-butyloxycarbonyl)acetamido-3-O-benzyl-2-deoxy-4-O-(2-naphthylmethyl)-α-L-altropyranosyluronate)-(1→3)-4-azido-2,4,6-trideoxy-2-trichloroacetamido-β-D-galactopyranoside (57). Di-tert-butylcarbonate (1.22 g, 5.6 mmol, 8.0 equiv.) followed by DMAP (34 mg, 282 μmol, 0.4 equiv.) were added to disaccharide 37 (640 mg, 704 μmol, 1.0 equiv.) in anhyd. THF (20 mL). After heating at 50° C. for 2 h, a TLC follow up (Tol/EtOAc 4:1) showed the presence of a less polar spot (R_{f} 0.75) and the absence of the starting 37 (R_{f} 0.1). The reaction mixture was allowed to reach rt and concentrated under reduced pressure. The residue was purified by flash chromatography (Tol/EtOAc 10:1→7:1) to give the desired 57 as a white solid (520 mg, 515 μmol, 73%). Disaccharide 57 had ¹H NMR (CDCl₃) δ 7.84-7.74 (m, 4H, H_{Ar}), 7.49-7.46 (m, 3H, H_{Ar}), 7.39-7.36 (m, 5H, H_{Ar}), 7.29-7.18 (m, 5H, H_{Ar}), 6.75 (d, J_{2,NH}=7.2 Hz, NH_{B}), 5.92-5.82 (m, 1H, CH_{All}), 5.72 (d, 1H, J_{1,2}=8.2 Hz, H-1_{A}), 5.29-5.23 (m, 1H, CH_{2All}), 5.22 (dₚₒ, 1H, CH_{2Bn-6}), 5.19 (dₚₒ, 1H, J=12.1 Hz, CH_{2Bn-6}), 5.20-5.16 (mₚₒ, 1H, CH_{2All}), 5.00-4.89 (br, 1H, H-2_{A}), 4.85 (dₚₒ, 1H, J=12.3 Hz, CH_{2Bn}), 4.81 (dₚₒ, 1H, CH_{2Bn}), 4.75 (dₚₒ, 1H, J_{1,2}=8.3 Hz, H-1_{B}), 4.72 (d, J_{4,5}=2.6 Hz, H-5_{A}), 4.42 (dₚₒ, 1H, J=11.8 Hz, CH_{2Nap}), 4.38 (ddₚₒ, 2H, J_{2,3}=10.5 Hz, J_{3,4}=2.3 Hz, H-3_{B}), 4.37 (ptₒ, 1H, J_{3,4}=2.8 Hz, H-4_{A}), 4.34-4.29 (m, 2H, CH_{2Nap}, CH_{2All}), 4.24 (brd, 1H, J_{2,3}=10.4 Hz, H-3_{A}), 4.05 (bd, 1H, H-4_{B}), 4.05-4.00 (m, 1H, CH_{2All}), 3.61-3.54 (pdt, 1H, H-2_{B}), 3.44 (dq, 1H, J_{4,5}=1.0 Hz, H-5_{B}), 2.34 (s, 3H, CH₃NAc), 1.48 (s, 9H, CH_{3NBoc}), 1.26 (d, 3H, J_{5,6}=6.2 Hz, H-6_{B}). ¹³C NMR (CDCl₃) δ 174.4 (br, CO_{NAc}), 169.1 (C-6_{A}), 161.7 (CO_{NTCA}), 153.9 (br, CO_{NBoc}), 137.8, 135.5, 135.0, 133.2, 132.9 (C_{q,Ar}), 133.6 (CH_{All}), 129.0, 128.8, 128.7 (2C), 128.2 (2C), 127.8, 127.6, 126.2, 126.0, 125.8, 125.7, 125.2 (10C, C_{Ar}), 117.9 (CH_{2All}), 98.9 (C-1_{A}, ¹J_{C,H}=174 Hz), 97.9 (C-1_{B}, ¹J_{C,H}=163 Hz), 92.4 (CCl₃), 83.7 (C_{Boc}), 76.9 (br, C-3_{B}), 74.1 (2C, C-4_{A}, C-5_{A}), 73.7 (br, C-3_{A}), 72.5 (CH_{2Nap}), 71.7 (CH_{2Bn}), 70.1 (CH_{2All}), 68.8 (C-5_{B}), 67.4 (CH_{2Bn-6}), 65.3 (C-4_{B}), 55.0 (C-2_{B}), 27.8 (CH_{3Boc}), 27.1 (CH_{3Ac}), 17.4 (C-6_{B}). HRMS (ESI⁺): m/z [M+NH₄]⁺ calcd for C₄₉H₅₈Cl₃N₆O₁₂, 1027.3179; found 1027.3176.

Allyl (benzyl 2-(N-tert-butyloxycarbonyl)acetamido-3-O-benzyl-2-deoxy-α-L-altropyranosyluronate)-(1→3)-4-azido-2,4,6-trideoxy-2-trichloroacetamido-β-D-galactopyranoside (58). DDQ (256 mg, 1.1 mmol, 3.0 equiv.) was added to the fully protected disaccharide 57 (380 mg, 377 μmol, 1.0 equiv.) in DCM/phosphate buffer pH 7 (10:1, 10 mL). After stirring vigorously at rt for 2 h, TLC analysis (Tol/EtOAc 4:1) revealed that the starting 57 (R_{f} 0.75) had evolved into a more polar product (R_{f} 0.35). 5% Aq. NaHCO₃ (10 mL) and DCM (10 mL) were added. The DCM layer was separated, washed with brine (25 mL), dried over Na₂SO₄, and concentrated. The residue was purified by flash chromatography (Tol/EtOAc 5:1→4:1) to give alcohol 58 as a white solid (260 mg, 257 μmol, 79%). Disaccharide 58 had ¹H NMR (CDCl₃) δ 7.44-7.40 (m, 5H, H_{Ar}), 7.32-7.22 (m, 5H, H_{Ar}), 6.65 (d, J_{2,NH}=7.6 Hz, NH_{B}), 5.91-5.81 (m, 1H, CH_{All}), 5.61 (d, 1H, J_{1,2}=8.0 Hz, H-1_{A}), 5.28-5.22 (mₚₒ, 1H, CH_{2All}), 5.25 (s, 2H, CH_{2Bn-6}), 5.19-5.15 (m, 1H, CH_{2All}), 4.71 (dₚₒ, 1H, J_{4,5}=2.6 Hz, H-5_{A}), 4.70-4.62 (br, 1H, H-2_{A}), 4.65 (d, 1H, J_{1,2}=8.4 Hz, H-1_{B}), 4.55-4.53 (mₚₒ, 1H, H-4_{A}), 4.54 (dₚₒ, 1H, CH_{2Bn}), 4.42 (d, 1H, J=11.6 Hz, CH_{2Bn}), 4.35 (dd, 1H, J_{2,3}=10.8 Hz, J_{3,4}=3.7 Hz, H-3_{B}), 4.33-4.27 (m, 2H, H-3_{A}, CH_{2All}), 4.04-3.99 (m, 2H, H-4_{B}, CH_{2All}), 3.61 (pt, 1H, H-2_{B}), 3.39 (dq, 1H, J_{4,5}=1.0 Hz, H-5_{B}), 2.59 (d, 1H, J_{4,OH}=2.2 Hz, OH), 2.31 (s, 3H, CH_{3Ac}), 1.50 (s, 9H, CH_{3Boc}), 1.25 (d, 3H, J_{5,6}=6.3 Hz, H-6_{B}). ¹³C NMR (CDCl₃) δ 174.1 (br, CO_{NAc}), 168.8 (C-6_{A}), 161.7 (CO_{NTCA}), 153.6 (br, CO_{NBoc}), 137.2, 135.0 (C_{q,Ar}), 133.6 (CH_{All}), 128.8 (2C), 128.5, 128.1, 127.8, (10C, C_{Ar}), 117.9 (CH_{2All}), 98.5 (C-1_{A}, ¹J_{C,H}=174 Hz), 98.1 (C-1_{B}, ¹J_{C,H}=161 Hz), 92.4 (CCl₃), 83.9 (OC_{Boc}), 76.8 (C-3_{B}), 75.2 (C-5_{A}), 73.3 (br, C-3_{A}), 72.0 (CH_{2Bn}), 70.1 (CH_{2All}), 68.7 (C-5_{B}), 67.6 (CH_{2Bn-6}), 66.8 (C-4_{A}), 65.3 (C-4_{B}), 54.9 (C-2_{B}), 27.8 (CH_{3Boc}), 27.1 (CH_{3Ac}), 17.3 (C-6_{B}). HRMS (ESI⁺): m/z [M+NH₄]⁺ calcd for C₃₈H₅₀Cl₃N₆O₁₂, 887.2552; found 887.2558.

(Benzyl 2-(N-tert-butyloxycarbonyl)acetamido-3-O-benzyl-2-deoxy-4-O-(2-naphthylmethyl)-α-L-altropyranosyluronate)-(1→3)-4-azido-2,4,6-trideoxy-2-trichloroacetamido-α/β-D-galactopyranose (59). A solution of [Ir(COD)(PMePh₂)₂]PF₆ (12 mg, 13 μmol, 0.03 equiv.) in anhyd. THF (3.0 mL), was degassed and stirred for 20 min under an H₂ atmosphere. The resulting yellow solution was degassed repeatedly with Ar and poured into a solution of allyl glycoside 57 (430 mg, 473 μmol, 1.0 equiv.) in anhyd. THF (16 mL). After stirring for 1 h at rt, NIS (117 mg, 520 μmol, 1.1 equiv.) and H₂O (4.0 mL) were added. After stirring for another hour at rt, a TLC follow up (CHex/EtOAc 6:1) showed the absence of allyl glycoside 57 (R_{f} 0.6) and products in close vicinity. 10% Aq. Na₂SO₃ was added, and volatiles were evaporated. The aq. phase was extracted with DCM (10 mL) twice. The combined organic layers were washed with brine (30 mL), dried over anhyd. Na₂SO₄, filtered, and concentrated under reduced pressure. Purification of the residue by flash chromatography (Chex/EtOAc 70:30→65:35) gave the expected hemiacetal 59 (360 mg, 356 μmol, 87%) as a white solid. Hemiacetal 59 had R_{f} 0.15, 0.25 (Tol/EtOAc, 3:1). ¹H NMR (major isomer, CDCl₃) δ 7.84-7.17 (m, 17H, H_{Ar}), 6.65 (d, 1H, J_{2,NH}=7.6 Hz, NH_{B}), 5.85 (d, 1H, J_{1,2}=8.4 Hz, H-1_{A}), 5.40 (brs, 1H, H-1_{B}), 5.24 (d, 1H, CH_{2Bn-6}), 5.16 (d, 1H, J=12.0 Hz, CH_{2Bn-6}), 5.05 (brs, 1H, H-2_{A}), 4.90 (d, 1H, CH_{2Nap}), 4.82 (d, 1H, J=12.3 Hz, CH_{2Nap}), 4.72 (d, 1H, J_{4,5}=2.4 Hz, H-5_{A}), 4.41 (d, 1H, J=11.6 Hz, CH_{2Bn}), 4.36 (dₚₒ, 1H, H-4_{A}), 4.35-4.26 (m, 2H, H-3_{B}, H-2_{B}), 4.20 (dd, 1H, J_{2,3}=10.5 Hz, J_{3,4}=2.1 Hz, H-3_{A}), 4.13 (brsₚₒ, 1H, H-4_{B}), 4.11 (bqₚₒ, 1H, H-5_{B}), 3.15 (brs, 1H, OH), 2.36 (s, 3H, CH_{3NAc}), 1.47 (sₚₒ, 9H, CH_{3Boc}), 1.22 (d, 3H, J_{5,6}=6.2 Hz, H-6_{B}). ¹³C NMR (major isomer, CDCl₃) δ 174.7 (br, CO_{NAcBoc}), 168.9 (C-6_{A}), 162.0 (CO_{NTCA}), 153.4 (br, CO_{Boc}), 137.7, 137.5, 135.4, 135.1, 134.8, 133.2, 133.0 (C_{q,Ar}), 128.9, 128.8, 128.7 (2C), 128.5, 128.3 (2C), 128.1, 127.8, 127.7, 126.3, 126.2, 126.0, 125.9, 125.7 (C_{Ar}), 98.0 (br, C-1_{A}, ¹J_{C,H}=174 Hz), 92.4 (CCl₃), 90.8 (C-1_{B}, ¹J_{C,H}=174 Hz), 84.1 (C_{Boc}), 74.2 (C-5_{A}), 74.0, 73.9 (2br, 3C, C-3_{B}, C-4_{A}, C-3_{A}), 72.7 (CH_{2Nap}), 71.6 (CH_{2Bn}), 67.4 (CH_{2Bn-6}), 65.6 (C-4_{B}), 64.6 (C-5_{B}), 55.4 (br, C-2_{A}), 51.1 (C-2_{B}), 27.8 (CH_{3NBoc}), 27.4 (CH_{3Ac}), 17.3 (C-6_{B}). HRMS (ESI⁺): m/z [M+NH₄]⁺ calcd for C₄₆H₅₈Cl₃N₇O₁₂, 987.2860; found 987.2862.

(Benzyl 2-(N-tert-butyloxycarbonyl)acetamido-3-O-benzyl-2-deoxy-4-O-(2-naphthylmethyl)-α-L-altropyranosyluronate)-(1→3)-4-azido-2,4,6-trideoxy-2-trichloroacetamido-α/β-D-galactopyranosyl)N-(phenyl)trifluoroacetamidate (60). PTFACl (23 μL, 148 μmol, 1.3 equiv.) and Cs₂CO₃ (197 mg, 604 μmol, 1.1 equiv.) were added to hemiacetal 59 (110 mg, 113 μmol, 1.0 equiv.) in acetone (4.0 mL). After stirring at rt for 2 h under an Ar atmosphere, a TLC follow up (Tol/EtOAc 6:1) indicated that hemiacetal 59 (R_{f} 0.1) had been converted to a less polar compound (R_{f} 0.85). The suspension was filtered over a pad of Celite, solids were washed with DCM (4 mL) twice, and volatiles were evaporated. The crude residue was purified by flash chromatography (Chex/EtOAc 90:10→98:12, 1% Et₃N) to give donor 60 (110 mg, 96 μmol, 86%) as a white solid. The donor had ¹H NMR (main isomer, CDCl₃) δ 7.85-7.10 (m, 21H, H_{Ar}), 6.79 (d, 2.2H, J=7.6 Hz), 6.53 (brs, 0.9H), 5.94 (d, 1H, J=8.4 Hz), 5.24 (d, 1H, CH_{2Bn-6}), 5.16 (d, 1H, J=12.0 Hz, CH_{2Bn-6}), 4.90 (brs, 2H, CH_{2Nap}), 4.74 (d, 1H, J=2.0 Hz, H-5_{A}), 4.37 (dₚₒ, 2H, H-2_{A}, CH_{2Bn}), 4.36-4.33 (m, 3H, H-3_{A}, H-4_{A}, H-4_{B}), 4.23 (dₚₒ, 1H, J=11.9 Hz, CH_{2Bn}), 4.22-4.17 (m, 2H, H-2_{B}, H-3_{B}), 3.97-3.91 (dq, 1H, H-5_{B}), 2.37 (s, 3H, CH_{3Ac}), 1.47 (s, 9H, CH_{3Boc}), 1.25 (d, 3H, H-6_{B}). ¹³C NMR (CDCl₃) δ 168.6, 162.1, 143.0, 138.0, 137.5, 135.0, 134.7, 133.2, 133.0 (C_{q,Ar}), 129.4, 129.0, 128.8, 128.7, 128.6 (2C), 128.5, 128.3, 128.2 (2C), 128.0, 127.9, 127.8 (2C), 127.6, 126.8, 126.3, 126.1, 126.0, 125.9, 125.7, 125.2, 124.4, 120.4, 119.3 (C_{Ar}), 107.3, 97.0, 93.4, 92.1, 84.4 (C_{Boc}), 77.2, 74.5 (C-5_{A}), 74.2 (2C), 72.5 (CH_{2Nap}), 71.6 (CH_{2Bn}), 67.7 (C-5_{B}), 67.5 (CH_{2Bn-6}), 64.5 (C-2_{A}), 50.3 (C-2_{B}), 27.9, 27.8, 27.3, 26.9, 17.6, 17.3. HRMS (ESI⁺): m/z [M+NH₄]⁺ calcd for C₅₄H₅₈C₁₃F₃N₇O₁₂, 1158.3156; found 1158.3137.

Allyl (benzyl 2-(N-tert-butyloxycarbonyl)acetamido-3-O-benzyl-2-deoxy-4-O-(2-naphthylmethyl)-α-L-altropyranosyluronate)-(1→3)-4-azido-2,4,6-trideoxy-2-trichloroacetamido-β-D-galactopyranyl)-(1→4)-(benzyl 2-(N-tert-butyloxycarbonyl)acetamido-3-O-benzyl-2-deoxy-α-L-altropyranosyluronate)-(1→3)-4-azido-2,4,6-trideoxy-2-trichloroacetamido-β-D-galactopyranoside (61). A mix of PTFA donor 60 (58 mg, 51 μmol, 1.1 equiv.) and acceptor 58 (40 mg, 46 μmol, 1.0 equiv.) was coevaporated with anhyd. toluene, dried under vacuum thoroughly, and taken into anhyd. DCE (2.0 mL) containing activated MS 4 Å (100 mg). The reaction mixture was stirred at rt for 30 min under an Ar atmosphere and cooled to 0° C. TfOH (0.2 μL, 0.05 equiv.) in 10 μL ACN was added. After stirring at this temperature for another 30 min, a TLC analysis (Tol/EtOAc 4:1) showed the absence of donor 60 and the presence of a new spot. Et₃N was added and solids were filtered off. Volatiles were evaporated and the crude was purified by flash chromatography (Tol/EtOAc 85:15→80:20) to give the desired 61 (35 mg, 19 μmol, 41%) as a white solid. Tetrasaccharide 61 had R_{f} 0.35 (Tol/EtOAc 4:1). ¹H NMR (CDCl₃) δ 7.83-7.75 (m, 5H, H_{Ar}), 7.48-7.17 (m, 24H, H_{Ar}), 6.85 (brs, 1H, NH_{B*}), 6.85 (brs, 1H, NH_{B1*}), 5.89-5.83 (m, 1H, CH_{All}), 5.73 (d, 1H, J_{1,2}=8.0 Hz, H-1_{A*}), 5.53 (brd, 1H, J_{1,2}=7.6 Hz, H-1_{A1*}), 5.28-5.15 (m, 6H, 2CH_{2Bn-6}, CH_{2All}), 5.03-4.88 (brs, 2H, H-2_{A*}, H-1_{B1}), 4.82 (brs, 3H, H-5_{A*}, CH_{2Nap}), 4.71-4.68 (m, 2H, H-5_{A1*}, H-1_{B}), 4.56-4.53 (m, 2H, H-2_{A1*}, H-3_{B*}), 4.44-4.21 (m, 9H, H-3_{B1*}, H-3_{A*}, H-3_{A1*}, H-4_{A*}, H-4_{A1*}, 2CH_{2Bn}), 4.07-4.00 (m, 3H, H-4_{B}, CH_{2All}), 3.64-3.38 (m, 1H, H-2_{B}, H-5_{B}), 2.38-2.22 (s, 6H, CH_{3Ac}), 1.52-1.43 (m, 18H, CH_{3Boc}), 1.29 (brd, 3H, J_{5,6}=6.4 Hz, H-6_{B*}), 1.19 (brd, 3H, J_{5,6}=6.0 Hz, H-6_{B1*}). ¹³C NMR (CDCl₃) δ 174.4 (CO_{NAcBoc}), 169.2, 168.7 (2C, C-6_{A}, C-6_{A1}), 161.9, 161.6 (2C, CO_{NTCA}), 153.8 (2C, CO_{NAcBoc}), 137.9, 137.8, 135.5, 135.1, 15.0, 133.2, 132.9 (C_{q, Ar}), 133.7 (CH_{All}), 129.0, 128.9, 128.8, 128.7, 128.6 (2C), 128.3, 128.2 (2C), 127.9, 127.8, 127.7, 127.6 (2C), 126.2, 126.0, 125.8, 125.7, 125.2 (10C, C_{Ar}), 117.6 (CH_{2All}), 99.4 (C-1_{B*}, ¹J_{C,H}=169 Hz), 98.9 (C-1_{A*}, ¹J_{C,H}=175 Hz), 98.5 (2C, C-1_{B1*}, ¹J_{C,H}=164 Hz, C-1_{A1*}, ¹J_{C,H}=177 Hz), 92.4 (CCl₃), 92.1 (CCl₃), 83.8 (C_{Boc}), 83.6 (C_{Boc}), 77.6, 77.2 (2C, C-3_{B}, C-3_{B1}), 76.0, 74.1 (2C, C-5_{A}, C-5_{A1}), 73.6 (2C, C-3_{A}, C-3_{A1}), 72.5 (CH_{2Nap}), 71.7, 71.6 (2C, CH_{2Bn}), 70.0 (CH_{2All}), 68.8 (2C, C-5_{B}, C-5_{B1}), 67.4, 67.3 (2C, CH_{2Bn-6}), 66.8 (2C, C-4_{A}, C-4_{A1}), 65.2 (2C, C-4_{B}, C-4_{B1}), 55.4 (2C, C-2_{A}, C-2_{A1}), 54.7 (2C, C-2_{B}, C-2_{B1}), 27.9, 27.8 (2C, CH_{3NBoc}), 27.4, 27.2 (2C, CH_{3Ac}), 17.4, 17.2 (2C, C-6_{B}, C-6_{B1}). HRMS (ESI⁺): m/z [M+NH₄]⁺ calcd for C₈₄H₉₈Cl₆N₁₁O₂₃ 1838.4963; found 1838.4982.

Propyl (2-acetamido-2-deoxy-α-L-altropyranosyluronic acid)-(1→3)-2-acetamido-4-amino-2,4,6-trideoxy-β-D-galactopyranoside (1).^{[1]} Route 1. TFA (15 μL, 197 μmol, 4 equiv.) was added to a solution of disaccharide 57 (46 mg, 49 μmol, 1.0 equiv.) in DCM (2 mL) at rt. After 3-4 h, a TLC follow up with Tol/EtOAc (4:1) showed the presence of a more polar single spot. Dilute aq. NaHCO₃ (5 mL) was added followed by DCM (5 mL). The DCM layer was separated, dried over Na₂SO₄, filtered, and concentrated under reduced pressure. 20% Pd(OH)₂/C (68 mg) was added to a solution of the crude intermediate in ^{t}BuOH/DCM/H₂O (16 mL, 7:3:1) and after extensive degassing, the atmosphere was saturated with hydrogen. After stirring under hydrogen for 48 h, the suspension was passed through a 0.2 μm filter and washed thoroughly with methanol. Volatiles were evaporated and the crude intermediate was dissolved in water (5 mL) and lyophilized. Purification by semi-preparative RP-HPLC gave the known propyl glycoside 1 as a white solid (16 mg, 35 μmol, 71%).

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranoside (2). Tetrasaccharide 61 (15 mg, 8.2 μmol, 1.0 equiv.), contaminated to a 15-20% extent by the disaccharide partners, was dissolved in DCM (1.0 mL) and TFA (15 μL, 197 μmol, 24 equiv.) was added. After stirring for 3 h at rt, a TLC follow up (Tol/EtOAc 2:1) indicated reaction completion. 10% Aq. NaHCO₃ (5 mL) and DCM (5 mL) were added. The organic layer was separated, dried over Na₂SO₄, filtered, and concentrated under reduced pressure. The crude was dissolved in ^{t}BuOH/DCM/H₂O (11 mL, 7:3:1) and 20% Pd(OH)₂/C (50 mg) was added. After stirring under an atmosphere of hydrogen for 48 h, the suspension was passed through a syringe filter (0.2 m) and washed thoroughly with methanol. The filtrate was evaporated and the crude was dissolved in water (5 mL) and lyophilized. The residue was purified by semi-preparative RP-HPLC to give the propyl glycoside 2 as a white foam (2.6 mg, 3.0 μmol, 37% (underestimated)). Tetrasaccharide 2 had RP-HPLC (215 nm) Rₜ=12.3 min (conditions A), Rₜ=13.6 min (conditions B), ¹H NMR (D₂O) δ 4.87 (d, 1H, J_{1,2}=8.4 Hz, H-1_{A}), 4.77 (dₚₒ, 1H, J_{1,2}=8.4 Hz, H-1_{A1}), 4.74 (dₚₒ, 1H, J_{1,2}=8.4 Hz, H-1_{A1}), 4.66 (brs, 1H, H-5_{A}), 4.44 (dₚₒ, 2H, J=8.4 Hz, H-1_{B1}, H-4_{A}), 4.36 (brs, 1H, H-4_{A1}), 4.15-4.08 (m, 2H, H-3_{B}, H-3_{B1}), 4.03-3.99 (mₚₒ, 2H, H-5_{B}, H-5_{B1}), 3.96-3.90 (m, 2H, H-2_{A}, H-4_{B}), 3.80-3.66 (m, 7H, H-3_{A}, H-3_{A1}, H-2_{A1}, H-2_{B}, H-2_{B1}, H-4_{B1}, OCH_{2Pr}), 3.51-3.49 (mₚₒ, 1H, OCH_{2Pr}), 1.99, 1.94 (2s, 12H, CH_{3Ac}), 1.51-1.48 (m, 2H, CH_{2Pr}), 1.29 (dₚₒ, 6H, H-6_{B}), 0.82 (t, 3H, J=7.2 Hz, CH_{3Pr}). ¹³C NMR (D₂O) δ 174.6, 174.5 (2C), 174.0 (CO_{NHAc}), 102.9 (C-1_{B1}, ¹J_{C,H}=168 Hz), 101.6 (C-1_{B}, ¹J_{C,H}=166 Hz), 101.1 (2C, C-1_{A}, C-1_{A1}, ¹J_{C,H}=170 Hz, ¹J_{C,H}=168 Hz), 79.7 (C-4_{A}), 76.5, 76.3 (C-5_{A}, C-5_{A1}), 76.0 (2C, C-3_{B}, C-3_{B1}), 72.7 (OCH_{2Pr}), 68.5 (C-4_{A1}), 68.5 (C-3_{A1}), 67.8 (C-3_{A}), 67.6 (C-4_{A}), 67.4, 67.3 (C-5_{B}, C-5_{B1}), 54.8 (C-4_{B}), 54.7 (C-4_{B1}), 51.5 (C-2_{A1}), 51.3 (C-2_{A}), 51.0, 50.8 (C-2_{B}, C-2_{B1}), 23.3, 22.2 (4C, CH_{3Ac}), 21.2 (CH_{2Pr}), 15.6, 15.5 (C-6_{B}, C-6_{B1}), 9.5 (CH_{3Pr}). HRMS (ESI⁺): m/z [M+Na]⁺ calcd for C₃₅H₅₈N₆O₁₉Na, 889.3649; found 889.3636.

### Example 3: Strategy 2_{A}-NAc₂,2_{B}-NTCA, 4_{A}-Nap Series

A Protecting Group Sensitive to Mild Base as Camouflage of the 2_{A}-Acetamide

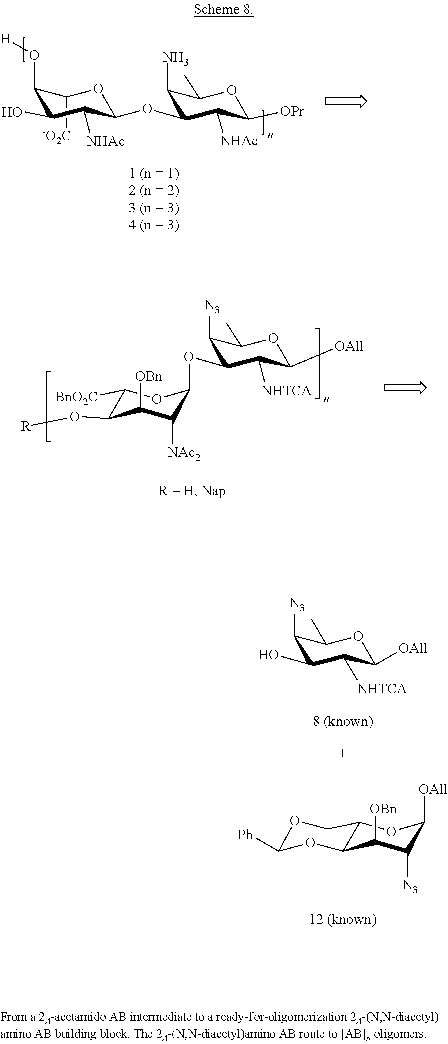

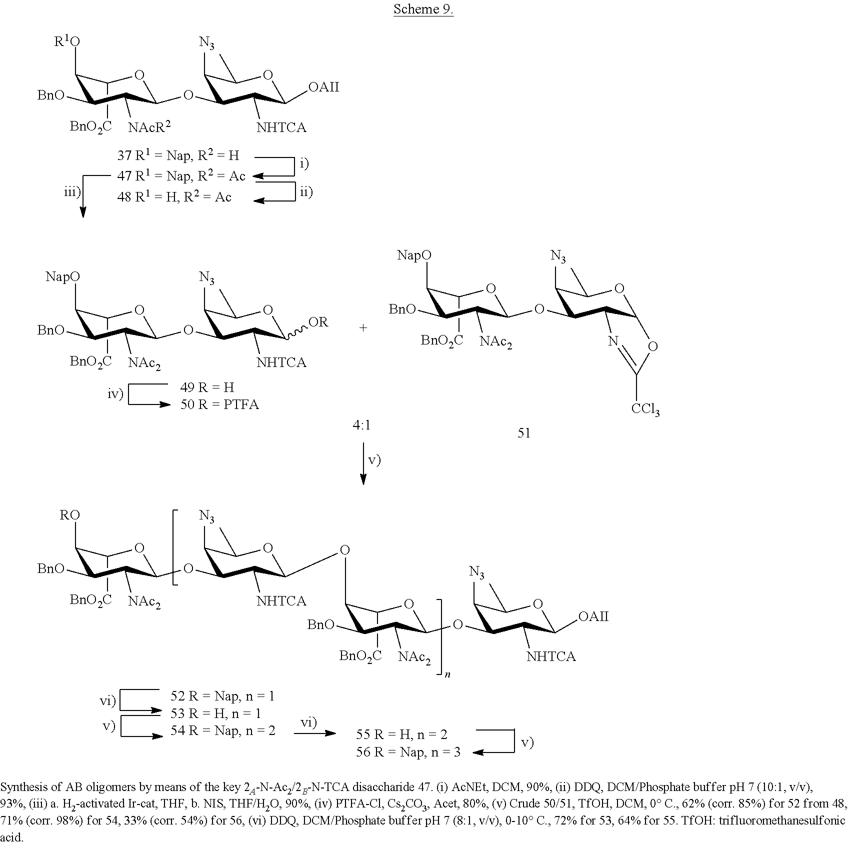

Allyl (benzyl 3-O-benzyl-2-deoxy-2-(N,N-diacetyl)amino-4-O-(2-naphthylmethyl)-α-L-altropyranosyluronate)-(1→3)-4-azido-2,4,6-trideoxy-2-trichloroacetamido-β-D-galactopyranoside (47). DIPEA (9.5 mL, 54.9 mmol, 20.0 equiv.) and acetyl chloride (3.9 mL, 21.9 mmol, 20 equiv.) were added successively to a solution of disaccharide 37 (2.5 g, 2.75 mmol, 1.0 equiv.) in anhyd. DCM (90 mL) at 0° C. The mixture was allowed to reach rt slowly and was stirred overnight. A TLC follow up (Tol/EtOAc 4:1) showed the complete conversion of acetamido 37 (R_{f} 0.25) to a less polar product (R_{f} 0.8). 10% Aq. NaHCO₃ (50 mL) was added and the biphasic mixture was diluted with DCM (20 mL). The organic layer was separated, dried over Na₂SO₄, and concentrated under reduced pressure. The residue was purified by flash chromatography (Tol/EtOAc 90:20→88:12) to give the fully protected 47 (2.36 g, 2.48 mmol, 90%) as an off-white solid. The N-acetylacetamido derivative 47 had R_{f} 0.65 (Tol/EtOAc 7:3). ¹H NMR (CDCl₃) δ 7.85-7.83 (m, 3H, H_{Ar}), 7.52-7.45 (m, 3H, H_{Ar}), 7.42-7.36 (m, 4H, H_{Ar}), 7.30-7.26 (m, 4H, H_{Ar}), 7.21-7.12 (m, 3H, H_{Ar}), 6.75 (d, J_{2,NH}=7.6 Hz, NH_{B}), 5.92-5.82 (m, 1H, CH_{All}), 5.80 (d, 1H, J_{1,2}=7.8 Hz, H-1_{A}), 5.29-5.23 (m, 1H, CH_{2All}), 5.26 (d, H, J=11.9 Hz, CH_{2Bn-6}), 5.21 (d, H, CH_{2Bn-6}), 5.20-5.17 (m, 1H, CH_{2All}), 4.85 (d, 1H, J=12.9 Hz, CH_{2Nap}), 4.81 (d, 1H, CH_{2Nap}), 4.76 (d, 1H, J_{1,2}=8.3 Hz, H-1_{B}), 4.73 (d, 1H, J_{4,5}=2.2 Hz, H-5_{A}), 4.48 (dd, 1H, J_{2,3}=10.8 Hz, J_{3,4}=3.8 Hz, H-3_{B}), 4.39-4.34 (mₚₒ, 3H, H-4_{A}, CH_{2Bn}), 4.35-4.30 (mₚₒ, 1H, CH_{2All}), 4.27 (ddₚₒ, 1H, J_{2,3}=10.5 Hz, J_{3,4}=2.9 Hz, H-3_{A}), 4.26 (dₚₒ, 1H, J=11.6 Hz, CH_{2Bn}), 4.06-4.00 (mₚₒ, 1H, CH_{2All}), 4.05 (bdₒ, 1H, H-4_{B}), 3.59 (ddd, 1H, H-2_{B}), 3.45 (dq, 1H, J_{5,6}=1.0 Hz, H-5_{B}), 2.38 (s, 6H, CH₃NAc), 1.27 (d, 3H, J_{5,6}=6.3 Hz, H-6_{B}). ¹³C NMR (CDCl₃) δ 175.1 (2C, CO_{NAc}), 168.7 (C-6_{A}), 161.9 (CO_{NTCA}), 137.8, 137.4, 135.2, 134.9, 133.2, 133.0 (C_{q,Ar}), 133.5 (CH_{All}), 128.7-125.2 (C_{Ar}), 117.9 (CH_{2All}), 98.8 (C-1_{A}, ¹J_{C,H}=176 Hz), 97.6 (C-1_{B}, ¹J_{C,H}=162 Hz), 92.3 (CCl₃), 77.4 (C-3_{B}), 73.9 (C-5_{A}), 73.7 (C-3_{A}), 73.0 (C-4_{A}), 72.5 (CH_{2Nap}), 71.9 (CH_{2Bn}), 70.2 (CH_{2All}), 68.7 (C-5_{B}), 62.6 (CH_{2Bn-6}), 65.2 (C-4_{B}), 59.1 (C-2_{A}), 55.2 (C-2_{B}), 21.4 (CH_{3Ac}), 17.4 (C-6_{B}). HRMS (ESI⁺): m/z [M+NH₄]⁺ calcd for C₄₆H₅₂Cl₃N₆O₁₁, 969.2759; found 969.2751.

Allyl (benzyl 3-O-benzyl-2-(N,N-diacetyl)amino-2-deoxy-α-L-altropyranosyluronate)-(1→3)-4-azido-2,4,6-trideoxy-2-trichloroacetamido-β-D-galactopyranoside (48). Disaccharide 47 (1.0 g, 1.0 mmol, 1.0 equiv.) was dissolved in DCM (10 mL) and phosphate buffer pH 7 (1.0 mL) was added. The biphasic mixture was cooled to 0° C. and DDQ (477 mg, 2.1 mmol, 2.0 equiv.) was added. The reaction was slowly allowed to reach rt and stirred for 3 h at this temperature. At completion, 5% aq. NaHCO₃ (30 mL) was added and the biphasic mixture was diluted with DCM (50 mL). The DCM layer was separated, washed with brine (100 mL), dried over Na₂SO₄, filtered, and concentrated under reduced pressure. The residue was purified by flash chromatography (Tol/EtOAc 5:1→4:1) to give alcohol 48 (800 mg, 0.93 mmol, 93%) as a white solid. Disaccharide 48 had R_{f} 0.45 (Tol/EtOAc, 7:3). ¹H NMR (CDCl₃) δ 7.45-7.39 (m, 5H, H_{Ar}), 7.35-7.25 (m, 3H, H_{Ar}), 7.20-7.17 (m, 2H, H_{Ar}), 6.69 (d, J_{2,NH}=7.4 Hz, NH_{B}), 5.90-5.80 (m, 1H, CH_{All}), 5.68 (d, 1H, J_{1,2}=7.9 Hz, H-1_{A}), 5.27 (s, 2H, CH_{2Bn-6}), 5.28-5.22 (m, 1H, CH_{2All}), 5.19-5.15 (m, 1H, CH_{2All}), 4.73 (d, 1H, J_{4,5}=2.3 Hz, H-5_{A}), 4.67 (d, 1H, J_{1,2}=8.3 Hz, H-1_{B}), 4.53-4.52 (mₚₒ, 1H, H-4_{A}), 4.50 (dₚₒ, 1H, CH_{2Bn}), 4.45 (ddₚₒ, 1H, J_{2,3}=10.7 Hz, J_{3,4}=3.8 Hz, H-3_{B}), 4.24 (dₚₒ, J=11.6 Hz, CH_{2Bn}), 4.36 (dd, 1H, J_{2,3}=10.3 Hz, J_{3,4}=3.4 Hz, H-3_{A}), 4.33-4.28 (m, 1H, CH_{All}), 4.07-3.99 (m, 3H, H-2_{A}, H-4_{B}, CH_{2All}), 3.60 (pdt, 1H, H-2_{B}), 3.39 (dqₚₒ, 1H, J_{4,5}=1.1 Hz, H-5_{B}), 2.57 (d, 1H, J_{4,OH}=2.0 Hz, OH), 2.37, 2.34 (2s, 6H, CH_{3NAc}), 1.24 (d, 3H, J_{5,6}=6.3 Hz, H-6_{B}). ¹³C NMR (CDCl₃) δ 174.9 (2C, CO_{N}Ae), 168.3 (C-6_{A}), 161.9 (CO_{NTCA}), 136.8, 134.9 (C_{q,Ar}), 133.5 (CH_{All}), 129.0, 128.9, 128.8, 128.6, 128.3, 128.2, 128.0 (C_{Ar}), 118.2 (CH_{2All}), 98.3 (C-1_{A}, ¹J_{C,H}=174 Hz), 97.7 (C-1_{B}, ¹J_{C,H}=162 Hz), 92.3 (CCl₃), 72.2 (C-3_{B}), 75.2 (C-5_{A}), 72.7 (C-3_{A}), 72.3 (CH_{2Bn}), 70.1 (CH_{2All}), 68.7 (C-5_{B}), 67.8 (CH_{2Bn-6}), 66.6 (C-4_{A}), 65.2 (C-4_{B}), 58.9 (C-2_{A}), 55.0 (C-2_{B}), 21.4 (CH_{3Ac}), 17.3 (C-6_{B}). HRMS (ESI⁺): m/z [M+NH₄]⁺ calcd for C₃₅H₄₄Cl₃N₆O₁₁, 829.2134; found 829.2128.

(Benzyl 3-O-benzyl-2-(N,N-diacetyl)amino-4-O-(2-naphthylmethyl)-2-deoxy-α-L-altropyranosyluronate)-(1→3)-4-azido-2,4,6-trideoxy-2-trichloroacetamido-α/β-D-galactopyranose (49). [Ir(COD)(PMePh₂)₂]PF₆ (18 mg, 0.02 mmol, 0.02 equiv.) in anhyd. THF (3.0 mL) was degassed and stirred for 20 min under an H₂ atmosphere. The resulting yellow solution was degassed repeatedly with Ar and poured into a solution of allyl glycoside 47 (1.0 g, 1.05 mmol, 1.0 equiv.) in anhyd. THF (20 mL). After stirring for 1 h at rt, a TCL follow up (cHex/EtOAc 10:1, 2 runs) revealed that the starting 47 (R_{f} 0.6) had been converted to a closely migrating product (R_{f} 0.65). NIS (260 mg, 1.1 mmol, 1.1 equiv.) and H₂O (12 mL) were added and after stirring for another 1 h at rt, 10% aq. Na₂SO₃ was added. The reaction mixture was concentrated and the aq. phase was extracted with DCM (30 mL) thrice. The combined organic layers were washed with brine (50 mL), dried over anhyd. Na₂SO₄, and concentrated under reduced pressure. The residue was purified by flash chromatography with cHex/EtOAc (80:20→75:25) to give the expected hemiacetal 49 (870 mg, 0.95 mmol, 90%) as a white solid. The α/β hemiacetal 49 had R_{f} 0.4, 0.45 (Tol/EtOAc, 4:1). The α isomer had ¹H NMR (CDCl₃) δ 7.85-7.75 (m, 4H, H_{Ar}), 7.52-7.46 (m, 3H, H_{Ar}), 7.39-7.26 (m, 8H, H_{Ar}), 7.21-7.10 (m, 2H, H_{Ar}), 6.70 (d, J_{2,NH}=9.2 Hz, NH_{B}), 5.80 (d, 1H, J_{1,2}=7.6 Hz, H-1_{A}), 5.24 (t, 1H, J_{1,2}=3.6 Hz, H-1_{B}), 5.19 (brs, 2H, CH_{2Bn-6}), 4.83 (brs, 2H, CH_{2Nap}), 4.74 (d, 1H, J_{4,5}=2.0 Hz, H-5_{A}), 4.42-4.33 (m, 3H, H-2_{A}, H-4_{A}, H-2_{B}), 4.34 (dₚₒ, 1H, J=12.0 Hz, CH_{2Bn}), 4.25 (dₚₒ, 1H, J=12.0 Hz, CH_{2Bn}), 4.23 (ddₚₒ, J_{3,4}=3.2 Hz, J_{2,3}=10.4 Hz, H-3_{A}), 4.15 (ddₚₒ, J_{3,4}=2.4 Hz, H-4_{B}), 4.09-4.03 (m, 2H, H-3_{B}, H-5_{B}), 3.15 (d, 1H, J_{1,OH}=3.6 Hz, OH), 2.39 (s, 3H, CH_{3Ac}), 2.38 (s, 3H, CH_{3Ac}), 1.21 (d, 3H, J_{5,6}=6.4 Hz, H-6_{B}). ¹³C NMR (CDCl₃), δ 175.1 (2C, CO_{NAc}), 168.8 (C-6_{A}), 161.9 (CO_{NTCA}), 135.1, 134.8, 133.2, 133.0 (C_{q,Ar}), 129.0, 128.8, 128.5, 128.4, 128.2, 127.9, 127.7 (2C), 126.4, 126.1, 125.6, 125.3 (C_{Ar}), 98.8 (C-1_{A}, ¹J_{C,H}=175 Hz), 92.4 (CCl₃), 91.2 (C-1_{B}, ¹J_{C,H}=176 Hz), 76.9 (C-3_{B}), 73.9 (C-5_{A}), 73.6 (C-4_{A}), 73.0 (C-3_{A}), 72.6 (CH_{2Nap}), 71.8 (CH_{2Bn}), 67.5 (CH_{2Bn-6}), 65.5 (C-4_{B}), 64.7 (C-5_{B}), 59.1 (C-2_{A}), 50.6 (C-2_{B}), 21.4 (CH_{3Ac}), 17.3 (C-6_{B}). HRMS (ESI⁺): m/z [M+NH₄]⁺ calcd for C₃₅H₄₄Cl₃N₆O₁₁, 1615.3589; found 1615.3596.

(Benzyl 3-O-benzyl-2-(N,N-diacetyl)amino-4-O-(2-naphthylmethyl)-2-deoxy-α-L-altropyranosyluronate)-(1→3)-4-azido-2,4,6-trideoxy-2-trichloroacetamido-α/β-D-galactopyranosyl (N-phenyl)trifluoroacetimidate (50) and 2-Trichloromethyl-[(Benzyl 3-O-benzyl-4-O-(2-naphthylmethyl)-2-deoxy-2-(N,N-diacetyl)amino-α-L-altropyranosyluronate)-(1→3)-4-azido-1,2,4,6-tetradeoxy-α-D-galactopyrano]-[2,1,d]-oxazoline (51). Hemiacetal 49 was dissolved in acetone (12 mL) and PTFACl (113 μL, 713 mol, 1.3 equiv.) was added followed by Cs₂CO₃ (197 mg, 604 μmol, 1.1 equiv.). After stirring at rt for 2 h, a TLC follow up (Tol/EtOAc 4:1) showed the complete conversion of the hemiacetal (R_{f} 0.4) into a less polar compound (R_{f} 0.9). The suspension was filtered over a pad of Celite, washed with acetone (5 mL) twice, and the filtrate was concentrated. The residue was purified by column chromatography (cHex/EtOAc 90:10→85:15, +1% Et₃N) to give a 4:1 mix of the expected PTFA donor 50 and oxazoline 51 (480 mg, 281 μmol, 80%) as a white solid. The isolated mix of 50 and 51 had R_{f} 0.9 (Tol/EtOAc 4:1). ¹H NMR (major compound, CDCl₃) δ 7.85-6.70 (m, 21H, H_{Ar}), 6.59 (d, 1H, J=8.4 Hz, NH), 5.94 (bs, 1H, H-1_{B}), 6.37 (d, 1H, J_{1,2}=8.0 Hz, H-1_{A}), 5.21 (bs, 2H, CH_{2Bn-6}), 6.37 (d, 1H, CH_{2Nap}), 6.37 (d, 1H, J=12.1 Hz, CH_{2Nap}), 4.88-4.81 (mₚₒ, 2.5H), 4.75 (d, 1H, J=2.0 Hz, H-5_{A}), 4.55 (ddd, 1H, H-2_{B}), 4.41 (ddₚₒ, 1H, J_{2,3}=10.5 Hz, H-2_{A}), 4.39-4.33 (m, 2H, H-4_{A}, CH_{2Bn}), 4.25 (d, 1H, J=11.8 Hz, CH_{2Bn}), 4.21 (ddₚₒ, 1H, J_{2,3}=2.8 Hz, H-3_{A}), 4.19 (do, 1H, H-4_{B}), 4.13 (d, 1H, J_{2,3}=11.0 Hz, J_{3,4}=3.2 Hz, H-3_{B}), 3.90 (brq, 1H, H-5_{B}), 2.38 (s, 6H, CH_{3Ac}), 1.25 (d, 3H, J_{5,6}=6.2 Hz, H-6_{B}). ¹³C NMR (major isomer, CDCl₃) δ 175.0 (CO_{NAc}), 168.8, 168.7 (C-6_{A}), 162.0 (CO_{NTCA}), 142.9, 137.2, 135.0, 134.7, 133.2, 133.0 (C_{q,Ar}), 128.9, 128.8, 128.7 (2C), 128.6, 128.4 (2C), 128.2 (2C), 128.0, 127.8, 127.7 (2C), 126.5, 126.4, 126.3, 126.2, 126.1, 126.0 (2C), 125.7, 124.9, 120.4, 119.2 (C_{Ar}), 98.5 (C-1_{A}), 93.7 (br, C-1_{B}), 92.0 (CCl₃), 76.3 (C-3_{B}), 74.1 (C-5_{A}), 73.5 (C-4_{A}), 73.0 (C-3_{A}), 72.6 (CH_{2Nap}), 71.8 (CH_{2Bn}), 67.6 (CH_{2Bn-6}), 67.5 (C-5_{B}), 64.6 (C-4_{B}), 59.0 (C-2_{A}), 49.9 (C-2_{B}), 29.6 (CH_{3Ac}), 17.3 (C-6_{B}). HRMS (ESI⁺): m/z [M+NH₄]⁺ calcd for C₅₁H₄₈Cl₃F₃N₆O₁₁, 1100.2737; found 1100.2729.

Allyl (benzyl 3-O-benzyl-2-(N,N-diacetyl)amino-2-deoxy-4-O-(2-naphthylmethyl)-α-L-altropyranosyluronate)-(1→-3)-(4-azido-2-trichloroacetamido-2,4,6-trideoxy-β-D-galactopyranosyl)-(1→3)-(benzyl 3-O-benzyl-2-(N,N-diacetyl)amino-2-deoxy-α-L-altropyranosyluronate)-(1→3)-4-azido-2-trichloroacetamido-2,4,6-trideoxy-β-D-galactopyranoside (52). PTFACl (102 μL, 642 μmol, 1.3 equiv.) and Cs₂CO₃ (117 mg, 543 μmol, 1.1 equiv.) were added to a solution of hemiacetal 49 (230 mg, 252 μmol, 1.0 equiv.) in acetone (8.0 mL). After stirring for 2 h at rt, the suspension was filtered over a pad of Celite and solids were washed with acetone (5 mL) thrice. The filtrate was concentrated under reduced pressure and the crude product was subjected to the next step.

The crude mix of glycosyl donors 50 and 51 (252 μmol theo., 1.1 equiv.) and acceptor 48 (184 mg, 227 μmol, 1.0 equiv.) were co-evaporated with anhyd. toluene (5 mL) and then dried under high vacuum for 1 h. The dried mixture was dissolved in anhyd. DCM (8.0 mL) and stirred for 1 h with freshly activated MS 4 Å (500 mg) under an Ar atmosphere. The reaction mixture was cooled to 0° C. and TfOH (1.1 μL, 13 μmol, 0.05 equiv.) was added. After stirring for 30 min at this temperature, a TLC analysis (Tol/EtOAc, 4:1) showed no further evolution while donor 50/51 (R_{f} 0.9) had reacted and a more polar spot (R_{f} 0.35) was visible. Et₃N (2.0 μL) was added and the suspension was filtered over a fitted funnel. Solids were washed with DCM (5 mL) twice and the filtrate was concentrated under reduced pressure. The residue was purified by flash chromatography (Tol/EtOAc 80:20→60:40) to give firstly the condensation product 52 (240 mg, 141 μmol, 62%; corr. yield 85%) as a white solid and then some unreacted 48 (50 mg, 7%). Tetrasaccharide 52 had ¹H NMR (CDCl₃) δ 7.82-7.74 (m, 4H, H_{Ar}), 7.51-7.10 (m, 23H, H_{Ar}), 6.99 (d, J_{2,NH}=6.8 Hz, NH_{B1}), 6.74 (d, J_{2,NH}=7.2 Hz, NH_{B}), 5.90 (m, 1H, CH_{All}), 5.78 (d, 1H, J_{1,2}=8.0 Hz, H-1_{A1}), 5.65 (d, 1H, J_{1,2}=8.0 Hz, H-1_{A}), 5.29-5.15 (m, 6H, CH_{2All}, CH_{2Bn-6}), 5.00 (d, 1H, J_{1,2}=8.0 Hz, H-1_{B1}), 4.85-4.79 (m, 3H, CH₂Nap, H-5_{A1}), 4.76 (d, 1H, J_{1,2}=8.0 Hz, H-1_{B}), 4.72 (d, 1H, J_{4,5}=2.4 Hz, H-5_{A}), 4.64 (dd, 1H, J_{3,4}=4.0 Hz, J_{2,3}=10.8 Hz, H-3_{B}), 4.45-4.22 (m, 11H, H-2_{A1}, H-3_{B1}, H-4_{A}, H-4_{A1}, H-3_{A}, H-3_{A1}, CH_{2All}, CH_{2Bn}), 4.07-3.99 (m, 4H, CH_{2All}, H-2_{A}, H-4_{B1}, H-4_{B}), 3.54-3.41 (m, 4H, H-2_{B}, H-2_{B1}, H-5_{B}, H-5_{B1}), 2.38 (brs, 12H, CH_{3Ac}), 2.23 (brs, 3H, CH_{3Ac}), 1.28 (d, 3H, J_{5,6}=6.0 Hz, H-6_{B}), 1.19 (d, 3H, J_{5,6}=6.4 Hz, H-6_{B1}). ¹³C NMR (CDCl₃) δ 175.3-174.8 (br, 4C, CO_{NAc}), 168.7, 168.3 (C-6_{A}, C-6_{A1}), 161.9, 161.8 (2CO_{NTCA}), 137.4, 135.2, 135.1, 135.0, 133.2, 133.0 (C_{q,Ar}), 133.5 (CH_{All}), 129.0, 128.9, 128.8, 128.7, 128.6, 128.5, 128.4, 128.2, 128.1, 128.0, 127.9, 127.8, 127.7, 127.6, 126.3, 126.1, 125.9, 125.6, 125.2 (C_{Ar}), 117.9 (CH_{2All}), 98.9 (C-1_{A1*}, ¹J_{C,H}=175 Hz), 98.8 (C-1_{B1}, ¹J_{C,H}=167 Hz), 98.3 (C-1_{A*}, ¹J_{C,H}=175 Hz), 97.6 (C-1_{B}, ¹J_{C,H}=163 Hz), 92.2, 91.8 (CCl₃), 76.7 (C-3_{B1}), 76.2 (C-5_{A1}), 75.9 (C-3_{B}), 73.4 (C-5_{A}), 73.6 (C-4_{A}), 72.8, 72.6 (C-3_{A}, C-3_{A1}), 72.5 (CH_{2Nap}), 72.0, 71.8 (CH_{2Bn}), 71.4 (C-4_{A1}), 70.1 (CH_{2All}), 68.7, 68.6 (C-5_{B}, C-5_{B1}), 67.5 (2C, CH_{2Bn-6}), 65.3 (C-4_{B}), 65.2 (C-4_{B1}), 59.3 (C-2_{A}), 59.1 (C-2_{A1}), 58.8 (C-2_{B1}), 55.3 (C-2_{B}), 27.7, 25.3, 21.4 (4C, CH_{3Ac}), 17.4, 17.2 (C-6_{B}, C-6_{B1}). HRMS (ESI⁺): m/z [M+NH₄]⁺ calcd for C₇₈H₈₆Cl₆N₆O₂₁ 1722.4131; found 1722.4110.

Allyl (benzyl 3-O-benzyl-2-(N,N-diacetyl)amino-2-deoxy-α-L-altropyranosyluronate)-(1→3)-(4-azido-2-trichloroacetamido-2,4,6-trideoxy-β-D-galactopyranosyl)-(1→3)-(benzyl 3-O-benzyl-2-(N,N-diacetyl)amino-2-deoxy-4-O-(2-naphthylmethyl)-α-L-altropyranosyluronate)-(1→3)-4-azido-2-trichloroacetamido-2,4,6-trideoxy-β-D-galactopyranoside (53). DDQ (108 mg, 475 μmol, 3.0 equiv.) was added to tetrasaccharide 52 (270 mg, 158 μmol, 1.0 equiv.) in DCM/phosphate buffer pH 7 (8:1, 18 mL) cooled to 0° C. The biphasic mixture was stirred vigorously for 4 h while allowing the bath to slowly warm to rt. At completion, 10% aq. NaHCO₃ (10 mL) was added followed by DCM (20 mL). The DCM layer was separated, washed with water and brine, dried over Na₂SO₄, filtered and concentrated under reduced pressure. The crude was purified by flash chromatography with Tol/EtOAc (75:25→70:30). Alcohol 53 (180 mg, 115 μmol, 73%), obtained as a white solid, had R_{f} 0.3 (Tol/EtOAc, 6:4). ¹H NMR (CDCl₃) δ 7.44-7.13 (m, 20H, H_{Ar}), 6.97 (d, J_{2,NH}=6.8 Hz, NH_{B1}), 6.80 (d, J_{2,NH}=7.2 Hz, NH_{B}), 5.88 (m, 1H, CH_{All}), 5.68 (d, 1H, J_{1,2}=7.8 Hz, H-1_{A1}), 5.64 (d, 1H, J_{1,2}=8.0 Hz, H-1_{A}), 5.34-5.15 (m, 6H, CH_{2All}, CH_{2Bn-6}), 4.95 (d, 1H, J_{1,2}=8.0 Hz, H-1_{B1}), 4.79 (d, 1H, J_{4,5}=2.0 Hz, H-5_{A1}), 4.76 (d, 1H, J_{1,2}=8.4 Hz, H-1_{B}), 4.72 (d, 1H, J_{4,5}=2.4 Hz, H-5_{A}), 4.64 (dd, 1H, J_{3,4}=4.0 Hz, J_{2,3}=10.4 Hz, H-3_{B}), 4.50-4.37 (m, 7H, H-3_{B1}, H-4_{A}, H-4_{A1}, H-3_{A}, H-3_{A1}, CH_{2Bn}), 4.32-4.24 (m, 3H, CH_{2Bn}, CH_{2All}), 4.13-3.99 (m, 4H, CH_{2All}, H-2_{A1}, H-2_{A}, H-4_{B1}, H-4_{B}), 3.54-3.41 (m, 4H, H-2_{B}, H-2_{B1}, H-5_{B}, H-5_{B1}), 2.61 (d, 1H, J_{4,OH}=2.0 Hz, OH), 2.38-2.32 (brs, 9H, CH_{3Ac}), 2.22 (brs, 3H, CH_{3Ac}), 1.28 (d, 3H, J_{5,6}=6.0 Hz, H-6_{B}), 1.19 (d, 3H, J_{5,6}=6.4 Hz, H-6_{B1}). ¹³C NMR (CDCl₃) δ 175.3, 174.7 (CO_{NAc}), 168.3, 168.2 (C-6_{A}), 161.9, 161.8 (CO_{NTCA}), 137.4, 136.9, 135.0 (2C) (C_{q,Ar}), 133.5 (CH_{All}), 129.0, 128.9, 128.7 (2C), 128.6 (2C), 128.5, 128.4, 128.3, 128.2, 128.0 (2C), 125.2 (C_{Ar}), 117.9 (CH_{2All}), 98.9 (C-1_{B1}, ¹J_{C,H}=168 Hz), 98.4 (C-1_{A}, ¹J_{C,H}=176 Hz), 98.4 (C-1_{A1}, ¹J_{C,H}=177 Hz), 97.6 (C-1_{B}, ¹J_{C,H}=162 Hz), 92.2, 91.9 (2C, CCl₃), 76.7 (C-3_{B1}), 76.2 (C-5_{A1}), 75.7 (C-3_{B}), 75.2 (C-5_{A}), 72.6 (C-4_{A}), 72.5, 71.4 (C-3_{A}, C-3_{A1}), 72.3, 72.0 (2C, CH_{2Bn}), 70.1 (CH_{2All}), 68.7, 68.6 (C-5_{B}, C-5_{B1}), 67.5, 67.5 (2C, CH_{2Bn-6}), 66.5 (C-4_{A1}), 65.3 (2C, C-4_{B}, C-4_{B1}), 59.3 (C-2_{A}), 59.1 (C-2_{A1}), 55.7 (C-2_{B1}), 55.2 (C-2_{B}), 27.7, 25.3, 21.4 (4C, CH_{3Ac}), 17.4, 17.2 (C-6_{B}, C-6_{B1}). HRMS (ESI⁺): m/z [M+NH₄]⁺ calcd for C₆₇H₇₈Cl₆N₁₁O₂₁ 1582.3505; found 1582.3503.

Allyl (benzyl 3-O-benzyl-2-(N,N-diacetyl)amino-2-deoxy-4-O-(2-naphthylmethyl)-α-L-altropyranosyluronate)-(1→3)-4-azido-2-trichloroacetamido-2,4,6-trideoxy-β-D-galactopyranosyl-(1→3)-(benzyl 3-O-benzyl-2-(N,N-diacetyl)amino-2-deoxy-α-L-altropyranosyluronate)-(1→3)-4-azido-2-trichloroacetamido-2,4,6-trideoxy-β-D-galactopyranosyl-(1→3)-(benzyl 3-O-benzyl-2-(N,N-diacetyl)amino-2-deoxy-α-L-altropyranosyluronate)-(1→3)-4-azido-2-trichloroacetamido-2,4,6-trideoxy-β-D-galactopyranoside (54). Hemiacetal 49 (131 mg, 144 μmol, 1.0 equiv.) was dissolved in acetone (7.0 mL). PTFACl (30 μL, 187 μmol, 1.3 equiv.) and Cs₂CO₃ (52 mg, 158 μmol, 1.1 equiv.) were added and the mixture stirred at rt for 2 h. Solids were filtered off over a pad of Celite and washed with acetone (5 mL) twice. The filtrate was concentrated under reduced pressure and the crude donor, isolated as a 4:1 mix of PTFA 50 and oxazoline 51, was subjected to the next step without further purification.

The crude mix of donors 50/51 (144 μmol theo., 1.25 equiv.) and acceptor 53 (180 mg, 115 μmol, 1.0 equiv.) were coevaporated with anhyd. toluene (5 mL) twice and then dried extensively under high vacuum. The mixture, dissolved in anhyd. DCM (6.0 mL), was stirred with freshly activated MS 4 Å (200 mg) for 30 min at rt under an Ar atmosphere, and cooled to 0° C. TfOH (1.0 μL, 0.05 equiv.) was added and after stirring for 30 min at this temperature, a TLC analysis indicated donor consumption and the presence of a major new product together with some unreacted acceptor. Et₃N (2.0 μL) was added and after 10 min, solids were filtered off and washed with DCM (5 mL) twice. Volatiles were evaporated and the residue was purified by flash chromatography (Tol/EA 75:25→60:40) to give first the condensation product 54 (200 mg, 81 μmol, 71%; corr. yield 98%) as a white solid, followed by some unreacted 53 (50 mg, 28%). Hexasaccharide 54 had R_{f} 0.25 (Tol/EtOAc 4:1). ¹H NMR (CDCl₃) δ 7.85-7.11 (m, 37H, H_{Ar}), 6.94 (d, J_{2,NH}=6.8 Hz, NH_{B2*}), 6.90 (d, J_{2,NH}=6.8 Hz, NH_{B1*}), 6.74 (d, J_{2,NH}=7.2 Hz, NH_{B}), 5.91-5.81 (m, 1H, CH_{All}), 5.78 (d, 1H, J_{1,2}=7.6 Hz, H-1_{A2}), 5.65 (d, 1H, J_{1,2}=7.8 Hz, H-1_{A1*}), 5.64 (d, 1H, J_{1,2}=7.8 Hz, H-1_{A*}), 5.31-5.16 (m, 8H, CH_{2All}, 3CH_{2Bn-6}), 5.03 (d, 1H, J_{1,2}=8.1 Hz, H-1_{B2*}), 4.99 (d, 1H, J_{1,2}=8.1 Hz, H-1_{B1*}), 4.82-4.80 (m, 2H, CH_{2Nap}), 4.80 (dₚₒ, 1H, J_{4,5}=2.2 Hz, H-5_{A*}) 4.78 (dₚₒ, 1H, J_{4,5}=2.4 Hz, H-5_{A1*}), 4.76 (dₚₒ, 1H, J_{1,2}=8.3 Hz, H-1_{B}), 4.72 (d, 1H, J_{4,5}=2.3 Hz, H-5_{A2*}), 4.68 (dd, 1H, J_{3,4}=4.0 Hz, J_{2,3}=10.7 Hz, H-3_{B2*}), 4.59 (dd, 1H, J_{3,4}=4.0 Hz, J_{2,3}=10.6 Hz, H-3_{B1*}), 4.44 (dd, 1H, J_{3,4}=3.8 Hz, J_{2,3}=10.7 Hz, H-3_{B}), 4.41-4.22 (m, 15H, H-2_{A2}, H-3_{B}, H-4_{A}, H-4_{A1}, H-4_{A2}, H-3_{A}, H-3_{A1}, H-3_{A2}, CH_{2All}, 3CH_{2Bn}), 4.09 (bdₚₒ, 1H, H-4_{B1*}), 4.07 (bdₚₒ, 1H, H-4_{B}), 4.05-3.99 (m, 4H, CH_{2All}, H-2_{A}, H-2_{A1}, H-4_{B2*}), 3.52 (dt, 1H, H-2_{B}), 3.50-3.43 (m, 3H, H-5_{B}, H-5_{B1}, H-5_{B2}), 3.42-3.36 (m, 2H, H-2_{B1}, H-2_{B2}), 2.41 (brs, 12H, 4CH_{3Ac}), 2.23 (brs, 6H, 2CH_{3Ac}), 1.30 (d, 3H, J_{5,6}=6.0 Hz, H-6_{B*}), 1.23 (d, 3H, J_{5,6}=6.4 Hz, H-6_{B1*}) 1.12 (d, 3H, J_{5,6}=6.4 Hz, H-6_{B2*}). ¹³C NMR (CDCl₃) δ 175.6, 175.3, 175.0, 174.7 (br, 6C, CO_{NAc}), 168.9, 168.8, 168.7 (3C, C-6_{A}), 162.3, 161.8, 161.7 (3C, CO_{NTCA}), 137.5, 137.4, 135.2, 135.1, 135.0 (C_{q,Ar}), 133.5 (CH_{All}), 133.2, 133.0 (C_{q,Ar}), 129.0, 128.9, 128.8, 128.7, 128.6, 128.5, 128.4, 128.2, 128.1, 128.0, 127.8, 127.7, 126.4, 126.1, 125.9, 125.6, 125.2 (27C, C_{Ar}), 117.9 (CH_{2All}), 98.9 (C-1_{B2*}, ¹J_{C,H}=167 Hz), 98.8 (C-1_{A2}, ¹J_{C,H}=175 Hz), 98.7 (C-1_{B1*}, ¹J_{C,H}=167 Hz), 98.4 (C-1_{A1*}, ¹J_{C,H}=177 Hz), 98.3 (C-1_{A*}, ¹J_{C,H}=177 Hz), 97.6 (C-1_{B}, ¹J_{C,H}=163 Hz), 76.7, 75.7, 75.1 (C-3_{B}, C-3_{B1}, C-3_{B2}), 76.2, 74.0 (3C, C-5_{A}, C-5_{A1}, C-5_{A2}), 73.7, 71.3 (3C, C-4_{A}, C-4_{A1}, C-4_{A2}), 72.8, 72.4, 70.9 (C-3_{A}, C-3_{A1}, C-3_{A2}), 72.5 (CH_{2Nap}), 72.0, 71.9 (3C, CH_{2Bn}), 70.1 (CH_{2All}), 68.6, 68.5 (C-5_{B}, C-5_{B1}, C-5_{B2}), 67.5 (3C, CH_{2Bn-6}), 65.3, 65.2 (C-4_{B}, C-4_{B1}, C-4_{B2}), 59.5, 59.4, 59.1 (C-2_{A}, C-2_{A1}, C-2_{A2}), 55.9, 55.2 (C-2_{B}, C-2_{B1}, C-2_{B2}), 27.7, 25.4 (3C, CH_{3Ac}), 17.4, 17.3 (C-6_{B}, C-6_{B1}, C-6_{B2}). HRMS (ESI⁺): m/z [M+2NH₄]²⁺ calcd for C₁₁₀H₁₂₄Cl₉N₁₇O₃₁ 1246.7922; found 1246.7922.

Allyl (benzyl 3-O-benzyl-2-deoxy-2-(N,N-diacetyl)amino-α-L-altropyranosyluronate)-(1→3)-(4-azido-2-trichloroacetamido-2,4,6-trideoxy-β-D-galactopyranosyl)-(1→3)-(benzyl 3-O-benzyl-2-deoxy-2-(N,N-diacetyl)amino-α-L-altropyranosyluronate)-(1→3)-(4-azido-2-trichloroacetamido-2,4,6-trideoxy-β-D-galactopyranosyl)-(1→3)-(benzyl 3-O-benzyl-2-deoxy-2-(N,N-diacetyl)amino-α-L-altropyranosyluronate)-(1→3)-4-azido-2-trichloroacetamido-2,4,6-trideoxy-β-D-galactopyranoside (55). DDQ (55 mg, 244 μmol, 3.0 equiv.) was added to hexasaccharide 54 (200 mg, 81 μmol, 1.0 equiv.) in DCM (8.0 mL) and phosphate buffer pH 7 (1.0 mL). The biphasic mixture was cooled to 0° C. and stirred for 2 h. Additional DDQ (200 mg, 81 μmol, 1.0 equiv.) was added and stirring was pursued for another 4 h while the bath temperature reached rt. A TLC analysis (Tol/EtOAC 3:1) showed the absence of the fully protected 54 (R_{f} 0.6) and the presence of a more polar spot (R_{f} 0.4). 10% Aq. NaHCO₃ (5 mL) was added followed by DCM (15 mL). The DCM layer was separated, washed with water and brine, dried over Na₂SO₄, filtered, and concentrated under reduced pressure. The residue was purified by flash chromatography (Tol/EtOAc 4:1→3:1) to give alcohol 55 (120 mg, 51 μmol, 64%) as a white solid. Hexasaccharide 55 had ¹H NMR (CDCl₃) δ 7.36-7.13 (m, 30H, H_{Ar}), 6.89 (dₚₒ, J_{2,NH}=6.8 Hz, NH_{B*}), 6.87 (d, J_{2,NH}=6.8 Hz, NH_{B1*}), 6.72 (d, J_{2,NH}=7.6 Hz, NH_{B2*}), 5.90-5.80 (m, 1H, CH_{All}), 5.68 (d, 1H, J_{1,2}=8.0 Hz, H-1_{A*}), 5.65 (dₚₒ, 1H, J_{1,2}=7.8 Hz, H-1_{A1*}), 5.65 (dₚₒ, 1H, J_{1,2}=7.8 Hz, H-1_{A2*}), 5.35-5.15 (m, 8H, CH_{2All}, 3CH_{2Bn-6}), 5.01 (dₚₒ, 1H, J_{1,2}=8.0 Hz, H-1_{B*}), 4.94 (d, 1H, J_{1,2}=8.4 Hz, H-1_{B1*}), 4.79-4.75 (m, 6H, CH₂Nap, H-5_{A*}, H-5_{A1*}, H-3_{B*}, H-1_{B2*}), 4.72 (d, 1H, J_{4,5}=2.4 Hz, H-5_{A2*}), 4.69 (dd, 1H, J_{3,4}=4.0 Hz, J_{2,3}=10.8 Hz, H-3_{B1*}), 4.58 (dd, 1H, J_{3,4}=4.0 Hz, J_{2,3}=10.4 Hz, H-3_{B2*}), 4.45-4.23 (m, 15H, H-3_{B}, H-4_{A}, H-4_{A1}, H-4_{A2}, H-3_{A}, H-3_{A1}, H-3_{A2}, CH_{2All}, 3CH_{2Bn}), 4.08-3.99 (m, 7H, H-2_{A}, H-2_{A1}, H-2_{A2}, H-4_{B}, H-4_{B1}, H-4_{B2}, CH_{2All}), 3.54-3.35 (m, 6H, H-2_{B}, H-2_{B1}, H-2_{B2}, H-5_{B}, H-5_{B1}, H-5_{B2}), 2.52 (d, 1H, J_{4,OH}=2.4 Hz, OH), 2.40-2.17 (brm, 18H, CH_{3Ac}), 1.29-1.12 (m, 12H, H-6_{B}, H-6_{B1}, H-6_{B2}). ¹³C NMR (CDCl₃) δ 175.3 (6C, CO_{NAc}), 168.3 (2C), 168.1 (3C, C-6_{A}), 161.8 (2C), 161.7 (3C, CO_{NTCA}), 133.5, 133.5 (CH_{All}), 137.4, 136.8, 135.0 (2C), 127.8, 127.0 (C_{q,Ar}), 129.0, 129.4, 128.8 (2C), 128.7 (2C), 128.6 (2C), 128.5 (2C), 128.4 (2C), 128.3, 128.2, 128.0 (2C), 125.2 (30C, C_{Ar}), 117.9 (CH_{2All}), 98.9 (C-1_{B*}, ¹J_{C,H}=166 Hz), 98.7 (C-1_{B1*}, ¹J_{C,H}=167 Hz), 98.4 (C-1_{A}, ¹J_{C,H}=175 Hz), 98.3 (2C, C-1_{A1*}, C-1_{A2*}, ¹J_{C,H}=175 Hz), 97.6 (C-1_{B2*}, ¹J_{C,H}=164 Hz), 92.6, 91.8 (3C, CCl₃), 76.6, 75.5, 75.1 (C-3_{B}, C-3_{B1}, C-3_{B2}), 76.2 (2C), 75.2 (C-5_{A}, C-5_{A1}, C-5_{A2}), 72.8, 72.6, 72.4 (C-3_{A}, C-3_{A1}, C-3_{A2}), 72.3, 72.0 (3C, 3CH_{2Bn}), 71.3, 71.0 (2C, C-4_{A}, C-4_{A1}), 70.1 (CH_{2All}), 68.6 (2C), 68.5 (C-5_{B}, C-5_{B1}, C-5_{B2}), 67.7, 67.5 (3C, CH_{2Bn-6}), 66.5 (C-4_{A2}), 65.4, 65.3, 65.2 (C-4_{B}, C-4_{B1}, C-4_{B2}), 59.5, 59.3, 59.0 (C-2_{A}, C-2_{A1}, C-2_{A2}), 55.8 (2C), 55.3 (C-2_{B}, C-2_{B1}, C-2_{B2}), 27.7 (6C, CH_{3Ac}), 17.4, 17.3, 17.2 (3C, C-6_{B}). HRMS (ESI⁺): m/z [M+NH₄]⁺ calcd for C₉₉H₁₁₂C₁₉N₁₆O₃₁ 2335.4876; found 2335.4871.

Allyl (benzyl 3-O-benzyl-2-deoxy-2-(N,N-diacetyl)amino-4-O-(2-naphthylmethyl)-α-L-altropyranosyluronate)-(1→3)-(4-azido-2-trichloroacetamido-2,4,6-trideoxy-β-D-galactopyranosyl)-(1→3)-(benzyl 3-O-benzyl-2-deoxy-2-(N,N-diacetyl)amino-α-L-altropyranosyluronate)-(1→3)-(4-azido-2-trichloroacetamido-2,4,6-trideoxy-β-D-galactopyranosyl)-(1→3)-(benzyl 3-O-benzyl-2-deoxy-2-(N,N-diacetyl)amino-α-L-altropyranosyluronate)-(1→3)-(4-azido-2-trichloroacetamido-2,4,6-trideoxy-β-D-galactopyranosyl)-(1→3)-(benzyl 3-O-benzyl-2-deoxy-2-(N,N-diacetyl)amino-α-L-altropyranosyluronate)-(1→3)-4-azido-2-trichloroacetamido-2,4,6-trideoxy-β-D-galactopyranoside (56). A 4:1 mix of donors 50/51 (92 mg, 85 μmol, 1.5 equiv.) and acceptor 55 (132 mg, 57 μmol, 1.0 equiv.) was coevaporated with toluene (3 mL) twice and dried extensively under high vacuum. The mixture, dissolved in anhyd. DCM (4.0 mL), was stirred for 45 min at rt with freshly activated MS 4 Å and cooled to 0° C. TfOH (3.8 μL, 0.05 equiv.) was added and the reaction mixture was stirred at this temperature. At completion, as revealed by TLC analysis (Tol/EtOAc 7:3), Et₃N (5.0 μL) was added and after 10 min, solids were filtered over a fitted funnel. The filtrate was concentrated to dryness and the residue was purified by flash chromatography (Tol/EtOAc 80:20→70:30) to give first the glycosylation product 56 (60 mg, 19 μmol, 33%, corr. yield, 54% wrt acceptor) as a white solid followed by the remaining unreacted acceptor 55 (50 mg, 38%). Octasaccharide 56 had R_{f} 0.35 (Tol/EtOAc 4:1). ¹H NMR (CDCl₃) δ 7.85-7.74 (m, 5H, H_{Ar}), 7.52-7.11 (m, 42H, H_{Ar}), 6.90-6.86 (m, 3H, NH_{B1}, NH_{B3}, NH_{B3}), 6.71 (d, 1H, J_{2,NH}=7.3 Hz, NH_{B}), 5.90-5.75 (m, 1H, CH_{All}), 5.78 (d, 1H, J_{1,2}=7.6 Hz, H-1_{A3}), 5.66-5.62 (m, 3H, H-1_{A}, H-1_{A1}, H-1_{A2}), 5.29-5.16 (m, 10H, CH_{2All}, 4CH_{2Bn-6}), 5.03-5.01 (m, 2H, H-1_{B1}, H-1_{B2}), 4.96 (d, 1H, J_{1,2}=8.0 Hz, H-1_{B3}), 4.83-4.80 (m, 3H, CH_{2Nap}, H-5_{A}), 4.78-4.76 (m, 3H, H-1_{B}, H-5_{A1*}, H-5_{A2*}), 4.72-4.55 (m, 4H, H-3_{B}, H-3_{B1}, H-3_{B2}, H-5_{A3*}), 4.45-4.22 (m, 17H, H-2_{A*}, H-3_{B3*}, H-3_{A}, H-3_{A1}, H-3_{A2}, H-3_{A3}, H-4_{A}, H-4_{A1}, H-4_{A2}, H-4_{A3}, CH_{2All}, 3CH_{2Bn}), 4.09-3.98 (m, 8H, CH_{2All}, H-2_{A1}, H-2_{A2}, H-2_{A3}, 4H-4_{B}), 3.55-3.32 (m, 8H, 4H-2_{B}, 4H-5_{B}), 2.40-2.19 (mₚₒ, 24H, 8CH_{3NAc}), 1.30-1.18 (d, 12H, J_{5,6}=6.4 Hz, H-6_{B}, H-6_{B1}, H-6_{B2}, H-6_{B3}). ¹³C NMR (Partial, CDCl₃) δ 175.5, 175.0, 174.6 (CO_{NAc}), 168.7 (2C), 168.3, 168.2 (C-6_{A}), 162.3, 161.8, 161.7 (4C, CO_{NTCA}), 137.4 (2C) 137.3, 135.2 (2C), 135.1, 135.0 (2C), 133.2, 133.0, 126.0 (C_{q,Ar}), 133.5 (CH_{All}), 129.0, 128.9, 128.8, 128.7 (2C), 128.6, 128.5 (2C), 128.4 (2C), 128.2, 128.1, 128.0, 127.8, 127.7, 126.4, 126.1, 125.9, 125.6, 125.2 (C_{Ar}), 117.9 (CH_{2All}), 98.9 (C-1_{A3*}, ¹J_{C,H}=178 Hz), 98.8 (C-1_{B3*}, ¹J_{C,H}=166 Hz), 98.7 (C-1_{B1}, C-1_{B2}, ¹J_{C,H}=168 Hz), 98.4 (C-1_{A1*}, C-1_{A2*}, J_{C,H}=178 Hz), 98.3 (C-1_{A*}, ¹J_{C,H}=178 Hz), 97.6 (C-1_{B}, ¹J_{C,H}=162 Hz), 92.2, 91.8 (4C, CCl₃), 76.6, 75.8, 75.1, 74.9 (4C, C-3_{B}, C-3_{B1}, C-3_{B2}, C-3_{B3}), 76.2, 73.7 (4C, C-5_{A}, C-5_{A1}, C-5_{A2}, C-5_{A3}), 74.0, 72.9, 72.8 (4C, C-4_{A}, C-4_{A1}, C-4_{A2}, C-4_{A3}), 72.5 (CH_{2Nap}), 71.8, 70.9 (3C, C-3_{A}, C-3_{A1}, C-3_{A2}), 72.0, 71.9 (4C, CH_{2Bn}), 70.1 (CH_{2All}), 68.6, 68.5 (4C, C-5_{B}, C-5_{B1}, C-5_{B2}, C-5_{B3}), 67.5 (4C, CH_{2Bn-6}), 65.4 (4C, C-4_{B}, C-4_{B1}, C-432, C-43), 59.5, 59.4, 59.1 (4C, C-2_{A}, C-2_{A1}, C-2_{A2}, C-2_{A3}), 55.8, 55.3 (4C, C-2_{B}, C-2_{B1}, C-2_{B2}, C-2_{B3}), 27.6, 25.2, 12.4 (8C, CH_{3Ac}), 17.4, 17.3, 17.2 (4C, C-6_{B}, C-4_{B1}, C-4_{B2}, C-4_{B3}). HRMS (ESI⁺): m/z [M+NH₄]⁺ calcd for C₁₄₂H₁₅₄Cl₁₂N₂₁O₄₁ 3229.6899; found 3230.7006.

Full Deprotection

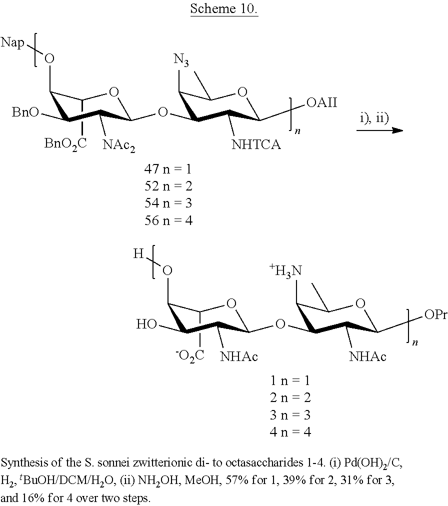

Propyl (2-acetamido-2-deoxy-α-L-altropyranosyluronic acid)-(1→3)-2-acetamido-4-amino-2,4,6-trideoxy-β-D-galactopyranoside (1).^{[1]} 20% Pd(OH)₂/C (100 mg) was added to a solution of 47 (50 mg, 53 μmol, 1.0 equiv.) in ^{t}BuOH/DCM/H₂O (17 mL, 20:5:2, v/v/v). After stirring for 48 h in a hydrogen atmosphere. The suspension was passed through a 0.2 μm filter and washed thoroughly with methanol. The filtrate was concentrated and the crude product was dried under vacuum. The obtained white powder was dissolved in methanol (5 mL) and hydroxylamine (3.7 mg, mol, 1.0 equiv.) was added. Monitoring by LCMS revealed the full consumption of the mono-acetate product and the presence of the desired product (LCMS: [M+H]⁺ m/z 867.2) after 4 h. Phosphate buffer pH 7 was added with frequent pH monitoring to achieve pH 7. The mixture was diluted with water (10 mL) and lyophilized. After freeze-drying, purification of the crude material by semi-preparative RP-HPLC gave propyl glycoside 1 as a white solid (14 mg, 30 μmol, 57%). Disaccharide 1 had RP-HPLC (215 nm) Rₜ=13.9 min (conditions B), Rₜ=12.2 min (conditions C). HRMS (ESI⁺): m/z calcd for C₁₉H₃₅N₃O₉Na [M+Na]⁺ m/z 486.2064; found 486.2067. NMR data were as published.^{[1]}.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranoside (2). Tetrasaccharide 54 (30 mg, 18 μmol, 1.0 equiv.) was dissolved in ^{t}BuOH/DCM/H₂O (10 mL, 20:5:2, v/v/v) and 20% Pd(OH)₂/C (100 mg) was added. The reaction mixture was degassed several times and stirred under a hydrogen atmosphere for 48 h. A follow up by HRMS revealed the presence of a major product corresponding the 2_{A}-NAc₂,2_{B}-NAc product (HRMS: C₃₉H₆₂N₆O₂₁Na [M+Na]⁺ m/z 973.4270). The suspension was passed through a syringe filter (0.2 m) and washed thoroughly with methanol. The filtrate was concentrated and the crude product was dried under vacuum. The resulting white powder was dissolved in methanol (3.0 mL) and hydroxylamine (2.2 mg, 36 μmol, 2.0 equiv.) was added. LCMS monitoring revealed that after 4 h, no intermediate remained and the desired product (LCMS: [M+H]⁺ m/z 867.3) was present to a large extent. The reaction mixture was neutralized with phosphate buffer with frequent pH monitoring to achieve pH 7, then diluted with water (6.0 mL) and lyophilized. Purification of the crude material by semi-preparative RP-HPLC gave the propyl glycoside 2 as a white solid (5.9 mg, 6.8 μmol, 39%). Tetrasaccharide 2 had RP-HPLC (215 nm) Rₜ=12.3 min (conditions A), Rₜ=13.6 min (conditions B), ¹H NMR (D₂O) δ 4.87 (d, 1H, J_{1,2}=8.4 Hz, H-1_{A}), 4.77 (dₚₒ, 1H, J_{1,2}=8.4 Hz, H-1_{A1}), 4.74 (dₚₒ, 1H, J_{1,2}=8.4 Hz, H-11), 4.66 (brs, 1H, H-5_{A}), 4.60 (brs, 1H, H-5_{A1}), 4.44 (dₚₒ, 2H, J=8.4 Hz, H-1_{B}, H-4_{A}), 4.36 (brs, 1H, H-4_{A1}), 4.15-4.08 (m, 2H, H-3_{B}, H-3_{B1}), 4.03-3.99 (mₚₒ, 2H, H-5_{B}, H-5_{B1}), 3.96-3.90 (m, 2H, H-2_{A}, H-4_{B}), 3.80-3.66 (m, 7H, H-3_{A}, H-3_{A1}, H-2_{A1}, H-2_{B}, H-2_{B1}, H-41, OCH_{2Pr}), 3.51-3.49 (mₚₒ, 1H, OCH_{2Pr}), 1.99, 1.94 (2s, 12H, CH_{3Ac}), 1.51-1.48 (m, 2H, CH_{2Pr}), 1.29 (dₚₒ, 6H, H-6_{B}, H-6_{B1}), 0.82 (t, 3H, J=7.2 Hz, CH_{3Pr}). ¹³C NMR (D₂O) δ 174.6, 174.5 (2C), 174.0 (4C, CO_{NHAc}), 172.7, 172.5 (2C, C-6_{A}, C-6_{A1}), 102.9 (C-1_{B1}, ¹J_{C,H}=168 Hz), 101.6 (C-1_{B}, ¹J_{C,H}=166 Hz), 101.1 (2C, C-1_{A}, C-1_{A1}, ¹J_{C,H}=170 Hz, ¹J_{C,H}=168 Hz), 76.7 (C-4_{A}), 76.5, 76.3, 76.0 (4C, C-5_{A}, C-5_{A1}, C-3_{B}, C-3_{B1}), 72.7 (OCH_{2Pr}), 68.5 (C-4_{A}), 67.8, 67.6 (2C, C-3_{A}, C-3_{A1}), 67.4, 67.3 (2C, C-5_{B}, C-5_{B1}), 54.8, 54.7 (2C, C-4_{B}, C-4_{B1}), 51.5 (C-2_{A1}), 51.3 (C-2_{A}), 51.0, 50.8 (2C, C-2_{B}, C-2_{B1}), 23.3, 22.2 (4C, CH_{3Ac}), 21.2 (CH_{2Pr}), 15.6, 15.5 (2C, C-6_{B}, C-6_{B}), 9.5 (CH_{3Pr}). HRMS (ESI⁺): m/z [M+Na]⁺ calcd for C₃₅H₅₈N₆O₁₉Na, 889.3649; found 889.3636.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (3). Hexasaccharide 54 (70 mg, 31 μmol, 1.0 equiv.) was dissolved in ^{t}BuOH/DCM/H₂O (19 mL, 20:5:2, v/v/v). 20% Pd(OH)₂/C (120 mg) was added and the suspension was degassed repeatedly. After stirring under a hydrogen atmosphere for 48 h, monitoring by LCMS analysis showed the presence of the targeted intermediate (LCMS: [M+H]⁺ m/z 1396.4). The suspension was passed through a 0.2 μm filter and washed extensively with methanol. The filtrate was concentrated and the crude material was dried under vacuum for 2 h. The obtained white powder was dissolved in methanol (3.0 mL) and hydroxylamine (6.1 mg, 85 μmol, 3.0 equiv.) was added. After stirring for 3 h, LCMS analysis showed the complete disappearance of the triacetate intermediate and the presence of the desired product (LCMS: [M+H]²⁺ m/z 635.2). Water (6.0 mL) was added and the reaction mixture was lyophilized. Purification of the crude material by semi-preparative RP-HPLC gave hexasaccharide 3 as a white solid (12 mg, 9.4 μmol, 31%). The propyl glycoside 3 had RP-HPLC (215 nm) Rₜ=11.3 min (conditions A), Rₜ=13.3 min (conditions B). ¹H NMR (D₂O) δ 4.88 (d, 1H, J_{1,2}=8.4 Hz, H-1_{A2}), 4.78 (dₚₒ, 2H, J_{1,2}=8.4 Hz, H-1_{A}, H-1_{A1}), 4.75 (dₚₒ, 1H, J_{1,2}=8.4 Hz, H-1_{B1}, H-1_{B2}), 4.70 (brsₚₒ, 2H, H-5_{A}, H-5_{A1}), 4.61 (brs, 1H, H-5_{A2}), 4.62 (mₚₒ, 2H, H-1_{B}, H-4_{A}), 4.36 (brs, 1H, H-4_{A1}), 4.16-4.09 (m, 3H, H-3_{B}, H-3_{B1}, H-3_{B2}), 4.06-3.90 (m, 7H, H-5_{B}, H-5_{B1}, H-5_{B2}, H-2_{A}, H-2_{A1}, H-4_{A}, H-4_{B}), 3.86-3.72 (m, 10H, H-3_{A}, H-3_{A1}, H-3_{A2}, H-4_{B1}, H-4_{B2}, H-2_{A2}, H-2_{B}, H-2_{B1}, H-2_{B2}, OCH_{2Pr}), 3.51-3.49 (mₚₒ, 1H, OCH_{2Pr}), 1.99, 1.98, 1.96, 1.95 (4 s, 18H, CH_{3Ac}), 1.54-1.45 (m, 2H, CH_{2Pr}), 1.30-1.29 (dₚₒ, 9H, H-6_{B}), 0.82 (t, 3H, J=7.2 Hz, CH_{3Pr}). ¹³C NMR (D₂O) δ 174.7, 174.5 (2C), 174.4 (2C), 174.0 (6C, CO_{NAcA,B}), 172.6, 172.2 (2C) (3C, C-6_{A}, C-6_{A1}, C-6_{A2}), 102.9 (2C, C-1_{B1}, C-1_{B2}, ¹J_{C,H}=174.2 Hz, ¹J_{C,H}=170.2 Hz), 101.6 (C-1_{B}, ¹J_{C,H}=162.0 Hz), 101.2, 101.1 (3C, C-1_{A}, C-1_{A1}, C-1_{A2}), 76.6 (2C, C-4_{A}, C-4_{A1}), 76.3, 76.1 (3C, C-5_{A}, C-5_{A1}, C-5_{A2}), 75.7 (3C, C-3_{B}, C-3_{B1}, C-3_{B2}), 72.7 (OCH_{2Pr}), 68.4 (C-4_{A2}), 68.2, 67.8 (3C, C-3_{A}, C-3_{A1}, C-3_{A2}), 67.6, 67.3 (3C, C-5_{B}, C-51, C-5_{B2}), 54.8, 54.7 (3C, C-4_{B}, C-4_{B1}, C-4_{B2}), 51.5, 51.3 (3C, C-2_{A}, C-2_{A1}, C-2_{A2}), 50.9, 50.8 (3C, C-2_{B}, C-2_{B1}, C-2_{B2}), 23.3, 22.2 (6C, CH_{3Ac}), 21.2 (CH_{2Pr}), 15.6 (3C, C-6_{B}, C-6_{B1}, C-6_{B2}), 9.5 (CH_{3Pr}). HRMS (ESI⁺): m/z [M+2H]²⁺ calcd for C₅₁H₈₅N₉O₂₈ 635.7747; found 635.7736.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (4). Octasaccharide 56 (20 mg, 12 μmol, 1.0 equiv.) was dissolved in ^{t}BuOH/DCM/H₂O (16.5 mL, 20:5:2, v/v/v). 20% Pd(OH)₂/C (50 mg) was added and the suspension was degassed repeatedly. After stirring under a hydrogen atmosphere for 48 h, the suspension was passed through a 0.2 μm filter and washed extensively with methanol. The filtrate was concentrated and the crude material was dried under vacuum for 2 h. HRMS analysis of the crude material showed the presence of the tetra-2-N-acetylacetamido product ([M+2H]²⁺ calculated for: C₇₅H₁₁₈N₁₂O₄₁ 921.3758, found 921.3753). The obtained white powder was dissolved in methanol (3.0 mL) and hydroxylamine (3.0 mg, 49 μmol, 4.0 equiv.) was added. After stirring for 6 h, LCMS analysis revealed the presence of a product of the desired mass (LCMS: [M+H]²⁺ m/z 837.2). Water (6.0 mL) was added and the reaction mixture was lyophilized. Purification of the crude material by semi-preparative RP-HPLC gave octasaccharide 4 as a white solid (1.7 mg, 1.01 μmol, 16%). The propyl glycoside 4 had RP-HPLC (215 nm) Rₜ=11.3 min (conditions A′). ¹H NMR (D₂O, 800 MHz) δ 4.81-4.79 (2dₚₒ, 3H, H-1_{A}), 4.67-4.65 (mₚₒ, 4H, H-1_{A}, 3H-1_{B}), 4.54 (brdₚₒ, 2H, H-4_{A}), 4.42-4.37 (brs, 4H, 3H-5_{A}, H-1_{B}), 4.31-4.30 (mₚₒ, 3H, H-5_{A}, 2H-4_{A}), 4.11-4.05 (m, 4H, H-3_{B}), 4.01-3.99 (q, 4H, H-5_{B}), 3.92-3.87 (m, 3H, H-2_{B}, 2H-4_{B}), 3.82-3.68 (m, 9H, 4H-2_{A}, 3H-2_{B}, 2H-4_{B}), 3.67-3.65 (mₚₒ, 3H, 2H-3_{A}, OCH_{2Pr}), 3.59-3.55 (mₚₒ, 3H, 2H-3_{A}, OCH_{2Pr}), 1.96-1.89 (mₚₒ, 24H, CH_{3Ac}), 1.47-1.44 (m, 2H, CH_{2P}r), 1.26 (mₚₒ, 12H, H-6_{B}), 0.78 (t, 3H, J=7.2 Hz, CH_{3Pr}). ¹³C NMR (D₂O, 800 MHz) δ 174.8, 174.6, 174.4, 173.9 (8C, CO_{NAc}), 173.9 (4C, C-6_{A}), 103.0 (2C, C-1_{A}), 101.6 (2C, C-1_{A}, C-1_{B}), 101.1 (2C, C-1_{B}), 100.9 (2C, C-1_{B}, C-1_{A}), 77.9, 77.4, 77.3 (3C, C-4_{A}, C-4_{A1}, C-4_{A2}), 76.0, 75.8, 75.7, 75.5 (4C, C-3_{B}), 72.6 (OCH_{2Pr}), 72.0 (C-4_{A3}), 69.1, 68.1, 67.7 (4C, C-3_{A}), 67.4, 67.2 (4C, C-5_{B}), 54.9, 54.7 (4C, C-4_{B}), 51.4 (4C, C-2_{A}), 50.9, 50.8 (4C, C-2_{B}), 22.3, 22.1 (8C, CH_{3Ac}), 21.2 (CH_{2Pr}), 15.5 (4C, C-6_{B}), 9.4 (CH_{3Pr}). HRMS (ESI⁺): m/z [M+2H]²⁺ calcd for C₆₁H₁₁₀N₁₂O₃₇ 837.3547; found 837.3542.

### Example 4: Strategy 2_{A}-NR¹R²,2_{B}-NDCA, 4_{A}-Nap Series

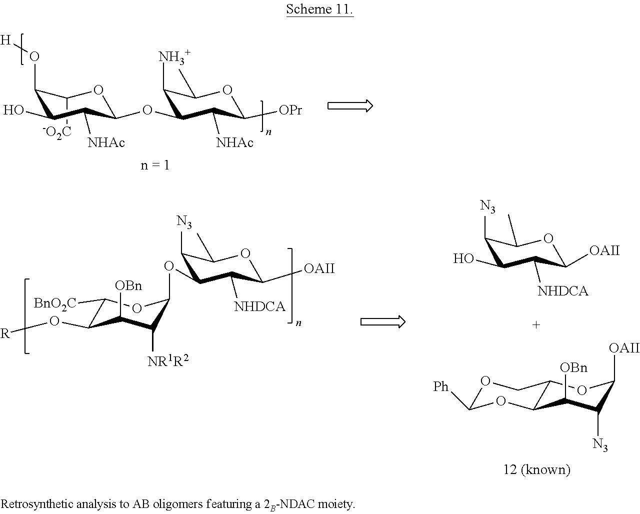

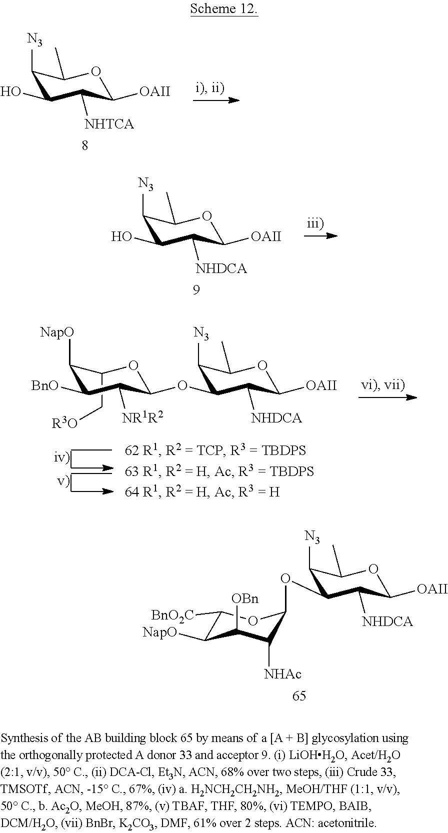

Allyl 4-azido-2-dichloroacetamido-2,4,6-trideoxy-β-D-galactopyranoside (9). LiOH·H₂O (304 mg, 7.2 mmol, 3.0 equiv.) was added to the fully protected 8 (1.0 g, 2.4 mmol, 1.0 equiv.) in acetone/water (3:1, 24 mL). After stirring for 2 h at 50° C., a TLC analysis (Tol/EtOAc 1:2) indicated the total consumption of the starting material (R_{f} 0.8) and the presence of a more polar product (R_{f} 0.0). The reaction mixture was concentrated under reduced pressure. The crude was passed through a short silica gel column eluting with 95:5 DCM/MeOH to give the intermediate amino alcohol after extensive drying under high vacuum. The latter had HRMS (ESI⁺): m/z [M+H]⁺ calcd for C₉H₁₆N₄O₃ 229.1301; found 229.1302.

Et₃N (0.5 mL, 3.6 mmol, 1.5 equiv.) was added to a solution of the crude amino alcohol in anhyd. ACN (10 mL), then cooled to 0° C. Dichloroacetyl chloride (391 μL, 2.6 mmol, 1.1 equiv.) was added slowly and after stirring at this temperature for 30 min, a follow up by TLC (Tol/EtOAc 7:3) indicated the total consumption of the amino alcohol and the presence of a less polar product (R_{f} 0.2). EtOAc (30 mL) and water (20 mL) were added and the organic layer was separated, dried over anhyd. Na₂SO₄, filtered, and concentrated under reduced pressure. The crude residue was purified by flash chromatography (Tol/EtOAc 7:3→6:4) to give the desired dichloroacetamide 9 (560 mg, 1.65 mmol, 68%) as a white solid. Acceptor 9 had ¹H NMR (DMSO-d₆) δ 8.40 (d, 1H, J_{NH,2}=9.2 Hz, NH), 6.38 (s, 1H, CHCl₂), 5.81-5.76 (m, 1H, CH_{All}), 5.63 (d, 1H, J=4.8 Hz, OH), 5.24-5.20 (m, 1H, CH_{2All}), 5.10-5.07 (m, 1H, CH_{2All}), 4.36 (d, 1H, J_{1,2}=8.4 Hz, H-1), 4.18-4.13 (m, 1H, CH_{2All}), 3.96-3.88 (m, 2H, H-3, CH_{2All}), 3.76 (dd, 1H, J_{3,4}=4.4 Hz, J_{4,5}<1.0 Hz, H-4), 3.67-3.62 (mₚₒ, 1H, H-5, H-2), 1.20 (d, 3H, J_{5,6}=6.4 Hz, H-6). ¹³C NMR (DMSO-d₆) δ 164.1 (CO_{NHDCA}), 134.9 (CH_{2All}), 116.7 (CH_{2All}), 100.5 (C-1, ¹J_{C,H}=161 Hz), 70.8 (C-3), 69.2 (CH_{2All}), 68.7 (C-5), 67.6 (CHCl₂), 66.2 (C-4), 53.3 (C-2), 17.7 (C-6). HRMS (ESI⁺): m/z [M+Na]⁺ calcd for C₁₁H₁₆Cl₂N₄O₄Na, 361.0446; found 361.0442.

Allyl 3-O-benzyl-2-deoxy-4-O-(2-napthylmethyl)-6-O-tert-butyldiphenylsilyl-2-tetrachlorophthalimido-α-L-altropyranosyl-(1→3)-4-azido-2-dichloroacetamido-2,4,6-trideoxy-β-D-galactopyranoside (62). The crude PTFA donor 33 (737 mg, 680 μmol, 1.15 equiv.) and acceptor 5 (200 mg, 592 μmol, 1.0 equiv.) were mixed and co-evaporated with toluene (5 mL) twice. The mixture was dried thoroughly under high vacuum for 1 h, dissolved in anhyd. ACN (15 mL) and stirred for 30 min with freshly activated MS 4 Å (1.0 g) under an Ar atmosphere. After cooling to −15° C., TMSOTf (8 μL, 34 μL, 0.05 equiv.) was added slowly. After stirring for 1 h at this temperature, a TLC analysis (Tol/EtOAc 9:1) revealed donor consumption and the presence of a new major spot (R_{f} 0.7). Et₃N (15 μL) was added and solids were filtered off. The filtrate was concentrated and the residue was purified by flash chromatography (cHex/EtOAc 90:10→85:15). Disaccharide 62 (490 mg, 397 μmol, 67%) was obtained as white solid. The coupling product had 62 had ¹H NMR (CDCl₃) δ 7.84-7.77 (m, 4H, H_{Ar}), 7.66-7.62 (m, 4H, H_{Ar}), 7.53-7.37 (m, 9H, H_{Ar}), 7.04-6.97 (m, 5H, H_{Ar}), 6.45 (d, 1H, J_{2,NH}=7.2 Hz, NH_{B}), 5.85-5.76 (m, 1H, CH_{All}), 5.67 (s, 1H, CHCl₂), 5.40 (d, 1H, J_{1,2}=7.2 Hz, H-1_{A}), 5.23-5.17 (m, 1H, CH_{2All}), 5.15-5.11 (m, 1H, CH_{2All}), 4.96 (d, 1H, J=12.6 Hz, CH_{2Nap}), 4.82 (dₚₒ, 1H, CH_{2Nap}), 4.82 (ddₚₒ, 1H, J_{2,3}=11.1 Hz, H-2_{A}), 4.62 (dₚₒ, 1H, J_{1,2}=8.4 Hz, H-1_{B}), 4.59 (d, 1H, J=12.4 Hz, CH_{2Bn}), 4.48 (dd, 1H, J_{3,4}=3.6 Hz, J_{2,3}=10.8 Hz, H-3_{B}), 4.34 (pdt, 1H, J_{4,5}=3.2 Hz, H-5_{A}), 4.29 (ddₚₒ, 1H, J_{3,4}=3.5 Hz, H-3_{A}), 4.27-4.22 (m, 1H, CH_{2All}), 4.12 (pt, H-4_{A}), 4.08 (d, 1H, CH_{2Bn}), 3.99-3.94 (m, 1H, CH_{2All}), 3.84 (dd, 1H, J_{6a,6b}=10.9 Hz, J_{5,6a}=6.3 Hz, H-6a_{A}), 3.80 (brdₚₒ, 1H, H-4_{B}), 3.78 (ddₚₒ, 1H, J_{5,6b}=6.2 Hz, H-6b_{A}), 3.50 (dddₚₒ, 1H, H-2_{B}), 4.47 (dqₚₒ, 1H, J_{4,5}=1.1 Hz, H-5_{B}), 1.20 (d, 3H, J_{5,6}=6.3 Hz, H-6_{B}), 1.03 (s, 9H, CH_{3TBDPS}). ¹³C NMR (CDCl₃) δ 163.9 (CO_{NTCA}), 163.3 (CO_{NTCP}), 139.8, 137.7, 135.7, 133.2, 133.0, 132.9, 129.5, 127.2 (C_{q,Ar}), 133.6 (CH_{All}), 135.6, 135.5, 129.9, 128.1, 128.0, 127.9 (2C), 127.8, 127.6, 127.5, 127.4, 126.5, 126.0, 125.9, 125.8 (C_{Ar}), 117.7 (CH_{2All}), 98.0 (C-1_{B}, ¹J_{C,H}=163 Hz), 97.8 (C-1_{A}, ¹J_{C,H}=170 Hz), 76.1 (C-5_{A}), 75.6 (C-3_{B}), 73.9 (C-3_{A}), 72.7 (CH_{2Nap}), 72.1 (CH_{2Bn}), 71.5 (C-4_{A}), 69.9 (CH_{2All}), 69.1 (C-5_{B}), 66.3 (CCl₃), 65.1 (C-4_{B}), 62.9 (C-6_{A}), 54.6 (C-2_{B}), 54.3 (C-2_{A}), 26.8 (CH_{3TBDPS}), 19.2 (C_{TBDPS}), 17.2 (C-6_{B}). HRMS (ESI⁺): m/z [M+NH₄]⁺ calcd for C₅₉H₆₁Cl₆N₆O₁₀Si, 1251.2350; found m/z 1251.2330.

Allyl 2-acetamido-3-O-benzyl-2-deoxy-4-O-(2-naphthylmethyl)-6-O-tert-butyldiphenylsilyl-α-L-altropyranosyl-(1→3)-4-azido-2-dichloroacetamido-2,4,6-trideoxy-β-D-galactopyranoside (63). Ethylenediamine (75 μL, 1.13 mmol, 4.0 equiv.) was added to disaccharide 62 (350 mg, 284 μmol, 1.0 equiv.) in THF/MeOH (1:1, 14 mL). The reaction mixture was heated at 50° C. for 60 h at which point, a TLC analysis (Tol/EtOAc 7:3) revealed the consumption of the starting 62 (R_{f} 0.95) and the presence of a new spot (R_{f} 0.1). After cooling to rt, Et₃N (0.5 mL) was added followed by Ac₂O (268 μL, 2.8 mmol, 10.0 equiv.). A TLC analysis (Tol/EtOAc 6:4) showed the presence of a new spot (R_{f} 0.4). Solids were filtered off and washed with DCM (5 mL) twice. The filtrate was concentrated and the crude product was purified by column chromatography (cHex/EtOAc 3:1→2:1). Disaccharide 63, obtained as a white solid (250 mg, 247 μmol, 87%), had ¹H NMR (CDCl₃) δ 7.85-7.63 (m, 8H, H_{Ar}), 7.51-7.18 (m, 15H, H_{Ar}), 6.68 (m, 1H, J_{2,NH}=7.2 Hz, NH_{B}), 5.94 (s, 1H, CHCl₂), 5.91-5.82 (m, 1H, CH_{All}), 5.29 (do, 1H, J_{2,NH}=7.7 Hz, NH_{A}), 5.28-5.24 (mₚₒ, 1H, CH_{2All}), 5.20-5.16 (m, 1H, CH_{2All}), 4.88 (d, 1H, J_{1,2}=8.4 Hz, H-1_{B}), 4.77 (d, 1H, J_{2,NH}=12.2 Hz, CH_{2Nap}), 7.73 (do, 1H, J_{1,2}=3.8 Hz, H-1_{A}), 4.72 (dₚₒ, 1H, CH_{2Nap}), 4.71 (do, 1H, CH_{2Bn}), 4.55 (d, 1H, J=12.2 Hz, CH_{2Bn}), 4.47-4.39 (m, 3H, H-2_{A}, H-3_{B}, H-5_{A}), 4.34-4.29 (m, 1H, CH_{2All}), 4.07-4.02 (m, 1H, CH_{2All}), 3.99 (ddₚₒ, 1H, J_{5,6a}=2.7 Hz, J_{6a,6b}=11.1 Hz, H-6a_{A}), 3.95 (ddₚₒ, 1H, J_{5,6b}=4.8 Hz, H-6b_{A}), 3.91 (dd, 1H, J_{2,3}=3.6 Hz, H-3_{A}), 3.65 (dd, 1H, J_{3,4}=3.6 Hz, J_{4,5}=8.8 Hz, H-4_{A}), 3.57 (brd, 1H, J_{3,4}=3.4 Hz, H-4_{B}), 3.53 (dq, 1H, J_{4,5}=1.1 Hz, H-5_{B}), 3.43 (ddd, 1H, J_{2,3}=10.8 Hz, H-2_{B}), 1.78 (s, 3H, CH_{3Ac}), 1.18 (d, 3H, J_{5,6}=6.3 Hz, H-6_{B}), 1.08 (s, 9H, CH_{3TBDPS}). ¹³C NMR (CDCl₃) δ169.3 (CO_{NDCA}), 164.7 (CO_{NAc}), 138.8, 135.2, 133.5 (2C), 133.1, 133.0 (C_{q,Ar}), 133.7 (CH_{All}), 135.7, 135.6, 12.7, 129.0, 128.2 (2C), 127.8, 127.7, 127.6 (2C), 126.7, 126.1, 125.9, 125.9 (C_{Ar}), 117.67 (CH_{2All}), 101.5 (C-1_{A}, ¹J_{C,H}=170 Hz), 97.8 (C-1_{B}, ¹J_{C,H}=163 Hz), 76.5 (C-3_{B}), 72.5 (C-3_{A}), 70.6 (C-4_{A}), 71.4 (CH_{2Nap}), 70.6 (CH_{2Bn}), 70.0 (CH_{2All}), 69.8 (C-5_{A}), 69.6 (C-5_{B}), 66.5 (CHCl₂), 65.6 (C-4_{B}), 63.7 (C-6_{A}), 55.3 (C-2_{B}), 49.8 (C-2_{A}), 27.0 (CH_{3TBDPS}), 23.1 (CH_{3Ac}), 19.4 (C_{TBDPS}), 17.1 (C-6_{B}). HRMS (ESI⁺): m/z [M+H]⁺ calcd for C₅₃H₆₂Cl₂N₅O₉Si, 1010.3694; found 1010.3669.

Allyl 2-acetamido-3-O-benzyl-2-deoxy-4-O-(2-naphthylmethyl)-α-L-altropyranosyl-(1→3)-4-azido-2-dichloroacetamido-2,4,6-trideoxy-β-D-galactopyranoside (64). TBAF (83 mg, 266 μmol, 1.2 equiv.) was added to disaccharide 63 (220 mg, 221 μmol, 1.0 equiv.) in anhyd. THF (10 mL) at rt. After 2 h, TLC monitoring (EtOAc), showed reaction completion and the presence of a more spot (R_{f} 0.1). Acetic acid (27 μL, 266 μmol, 1.2 equiv.) was added. Volatiles were removed under reduced pressure. The residue was purified by flash chromatography (EtOAc/MeOH 100:0→85:15) to give alcohol 64 as a white solid (135 mg, 175 μmol, 80%). Disaccharide 64 had ¹H NMR (DMSO-d₆) δ 8.57 (d, 1H, J_{2,NH}=9.2 Hz, NH_{B}), 7.94-7.82 (m, 4H, HA, NH_{A}), 7.76 (brs, 1H, H_{Ar}), 7.51-7.49 (m, 2H, H_{Ar}), 7.42-7.39 (m, 3H, H_{Ar}), 7.32-7.25 (m, 3H, H_{Ar}), 6.44 (s, 1H, CHCl₂), 5.84-5.76 (m, 1H, CH_{All}), 5.26-5.21 (m, 2H, CH_{2All}), 5.12-5.09 (m, 1H, CH_{2All}), 4.76 (brs, 1H, H-1_{A}), 4.71 (d, 1H, J=11.6 Hz, CH_{2Nap}), 4.66-4.61 (m, 2H, CH_{2Bn}, OH), 4.55 (d, 1H, J=11.6 Hz, CH_{2Bn}), 4.50 (dₚₒ, 1H, CH_{2Nap}), 4.48 (d, 1H, J_{1,2}=8.0 Hz, H-1_{B}), 4.28-4.15 (m, 3H, H-2_{A}, H-5_{A}, CH_{2All}), 4.03-3.94 (m, 3H, H-3_{B}, H-4_{B}, CH_{2All}), 3.82-3.69 (m, 5H, H-2_{B}, H-5_{B}, H-3_{A}, H-4_{A}, H-6a_{A}), 3.56-3.50 (m, 1H, H-6b_{A}), 1.78 (s, 3H, CH_{3Ac}), 1.23 (d, 3H, J_{5,6}=6.4 Hz, H-6_{B}). ¹³C NMR (DMSO-d₆) δ 169.3 (CO_{NDCA}), 164.2 (CO_{NAc}), 139.3, 136.5, 133.2, 132.9 (C_{q,Ar}), 134.9 (CH_{All}), 128.3, 128.2, 128.1, 128.0, 127.5, 126.5, 126.4, 126.3 (2C) (C_{Ar}), 116.8 (CH_{2All}), 101.8 (C-1_{A}, ¹J_{C,H}=169 Hz), 100.1 (C-1_{B}, ¹J_{C,H}=161 Hz), 77.7 (C-3_{B}), 73.6 (C-3_{A}), 72.6 (C-4_{A}), 70.9 (CH_{2Nap}), 70.3 (CH_{2Bn}), 69.8 (C-5_{A}), 69.5 (C-5_{B}), 69.2 (CH_{2All}), 67.6 (CHCl₂), 65.4 (C-4_{B}), 61.5 (C-6_{A}), 52.4 (C-2_{B}), 49.5 (C-2_{A}), 22.9 (CH_{3Ac}), 17.6 (C-6_{B}). HRMS (ESI⁺): m/z 772.2502 (calcd for C₃₇H₄₃Cl₂N₅O₉H [M+H]⁺ m/z 772.2516).

Allyl (benzyl 2-acetamido-3-O-benzyl-6-O-benzyl-4-O-(2-naphthylmethyl)-2-deoxy-α-L-altropyranosyluronate)-(1→3)-4-azido-2-dichloroacetamido-2,4,6-trideoxy-β-D-galactopyranoside (65). TEMPO (4 mg, 26 μmol, 0.2 equiv.), followed by BAIB (104 mg, 324 mol, 2.5 equiv.), were added to a suspension of alcohol 64 (100 mg, 130 μmol, 1.0 equiv.) in DCM/water (2:1, 6.0 mL) at rt. After stirring vigorously for 2 h at rt, TLC monitoring (EtOAc/MeOH 20:1) showed the presence of a major polar spot and absence of the starting 64 (R_{f} 0.2). 50% Aq. Na₂SO₃ (5 mL) was added, the DCM layer was separated, and the water phase was extracted with chloroform/isopropanol (3:1, 10 mL) twice. The water phase was acidified with dilute aq. HCl to reach pH ˜1 and again extracted with chloroform/isopropanol (3:1, 10 mL) twice. The combined organic phases were washed with brine (50 mL), dried by passing through a phase separator filter and concentrated under reduced pressure. The crude thus obtained was dissolved in DMF (2.0 mL) rt and benzyl bromide (44 μL, 259 μmol, 2.0 equiv.) followed by K₂CO₃ (27 mg, 194 μmol, 1.5 equiv.) were added. After stirring for 4 h at rt, water (20 mL) was added. The aq. layer was washed with DCM (5.0 mL) three times. The combined DCM parts were washed with brine (20 mL), dried over Na₂SO₄, and concentrated under reduce pressure. The crude was purified by flash chromatography with (Tol/EtOAc 70:30→65:35). The benzyl ester 65, obtained as an off-white solid (70 mg, 79 μmol, 61%), had R_{f} 0.2 (Tol/EtOAc 7:3). ¹H NMR (CDCl₃) δ 7.84-7.68 (m, 5H, H_{Ar}), 7.51-7.25 (m, 12H, H_{Ar}), 6.78 (d, J_{2,NH}=7.2 Hz, NH_{B}), 6.04 (s, 1H, CHCl₂), 5.91-5.81 (m, 2H, NH_{A}, CH_{All}), 5.37 (d, 1H, J_{1,2}=6.0 Hz, H-1_{A}), 5.28-5.13 (mₚₒ, 4H, CH_{2All}, CH_{2Bn-6}), 4.75 (d, 1H, J_{4,5}=4.0 Hz, H-5_{A}), 4.73 (dₚₒ, 1H, J_{1,2}=8.8 Hz, H-1_{B}), 4.73 (dₚₒ, 1H, CH_{2Nap}), 4.68 (d, 1H, J=12.4 Hz, CH_{2Nap}), 4.51 (dₚₒ, 1H, J=12.0 Hz, CH_{2Bn}), 4.48 (dₚₒ, 1H, CH_{2Bn}), 4.47 (ddₚₒ, J_{3,4}=3.8 Hz, J_{2,3}=10.9 Hz, H-3_{B}), 4.33-4.28 (m, 1H, CH_{2All}), 4.11-4.01 (m, 2H, H-3_{A}, H-4_{A}, CH_{2All}), 3.95-3.90 (m, 2H, H-2_{A}, H-4_{B}), 3.52 (pdt, 1H, H-2_{B}), 3.43 (q, 1H, H-5_{B}), 1.88 (s, 3H, CH_{3Ac}), 1.24 (d, 3H, J_{5,6}=6.4 Hz, H-6_{B}). ¹³C NMR (CDCl₃), δ170.6 (C-6_{A}), 169.3 (CO_{NDCA}), 164.3 (CO_{NAc}), 137.9, 134.9, 134.8, 133.1, 133.0 (C_{q,Ar}), 133.6 (CH_{All}), 133.1, 133.0 (C_{q,Ar}), 128.7 (2C), 128.6, 128.3, 128.2, 128.1, 127.9, 127.8, 127.6, 126.8, 126.1, 126.0, 125.9 (17C, C_{Ar}), 117.9 (CH_{2All}), 99.3 (C-1_{A}, ¹J_{C,H}=170. Hz), 97.9 (C-1_{B}, ¹J_{C,H}=162 Hz), 77.0 (C-3_{B}), 73.4 (C-3_{A}), 73.1 (C-4_{A}), 72.1 (C-5_{A}), 72.0 (2C, CH_{2Bn},Naₚ), 70.1 (CH_{2All}), 69.2 (C-5_{B}), 67.5 (CH_{2Bn-6}), 66.7 (CHCl₂), 65.2 (C-4_{B}), 54.3 (C-2_{B}), 52.5 (C-2_{A}), 23.4 (CH_{3Ac}), 17.3 (C-6_{B}). HRMS (ESI⁺): m/z [M+H]⁺ calcd for C₄₄H₄₈Cl₂N₅O₁₀, 876.2778; found 876.2773.

### Example 5: Strategy 2_{A}-NTCA,2_{B}-NTCA, TBS Series

AB Building Block for Oligomerization: 4_{A}-TBS

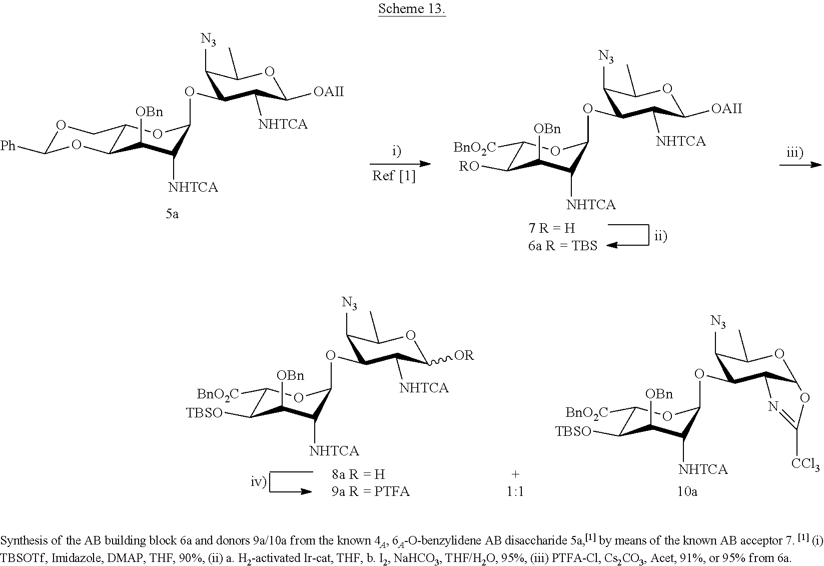

Allyl (benzyl 3-O-benzyl-4-O-tert-butyldimethylsilyl-2-deoxy-2-trichloroacetamido-α-L-altropyranosyluronate)-(1→3)-4-azido-2-trichloroacetamido-2,4,6-trideoxy-β-D-galactopyranoside (6a). Imidazole (1.0 g, 14.7 mmol, 5.0 equiv.) and DMAP (50 mg, 290 nmol, 0.1 equiv.) were added to a solution of alcohol 7a (2.57 g, 2.94 mmol, 1.0 equiv.) in anhyd. THF (11.8 mL). The reaction mixture was cooled to 0° C. and tert-butyldimethylsilyl trifluoromethanesulfonate (1.69 mL, 7.35 mmol, 2.5 equiv.) was added slowly. After stirring for 2 h at rt, a TLC follow up (Tol/EtOAc 8:2) showing conversion of the starting 7a into a less polar product indicated reaction completion. MeOH (1.5 mL) was added. After stirring for 15 min at rt, volatiles were evaporated and the residue was purified by flash chromatography (cHex/EtOAc 90:10→80:20) to give the fully protected 6a (2.62 g, 2.65 mmol, 90%). Disaccharide 6a had R_{f} 0.3 (Tol/EtOAc 8:2). HRMS (ESI⁺): m/z [M+Na]⁺ calcd for C₃₉H₄₉Cl₆N₅O₁₀SiNa, 1008.1277; found 1008.1379.

(Benzyl 3-O-benzyl-4-O-tert-butyldimethylsilyl-2-deoxy-2-trichloroacetamido-α-L-altropyranosyluronate)-(1→3)-4-azido-2-trichloroacetamido-2,4,6-trideoxy-β-D-galactopyranose (8a). [Ir(COD)(PMePh₂)₂]PF₆ (27 mg, 30 nmol, 0.02 equiv.) was dissolved in anhyd. THF (3.2 mL) and stirred for 20 min under an H₂ atmosphere. The resulting yellow solution was degassed repeatedly with Ar and transferred by means of a cannula into a solution of allyl glycoside 6a (1.58 g, 1.6 mmol, 1.0 equiv.) in anhyd. THF (8.0 mL). The reaction mixture was stirred for 2 h at rt, at which time a solution of I₂ (811 mg, 3.2 mmol, 2.0 equiv.) in THF/H₂O (4:1, 7.8 mL) was added. After stirring for 45 min at rt, a TLC analysis (Tol/EtOAc 95:5) revealed the full consumption of the isomerization product (R_{f} 0.4) and the presence of two more polar spots (R_{f} 0.25, 0.1). 10% Aq. Na₂SO₃ (50 mL, 20 equiv.) was added and volatiles were evaporated. The aq. phase was extracted with DCM (200 mL) twice. The combined organic layers were washed with water and brine, dried over Na₂SO₄, filtered, and concentrated under vacuum. Purification of the residue by flash chromatography (cHex/EtOAc 80:20→40:60) gave the expected hemiacetal 8a (1.42 g, 1.5 mmol, 95%) as a white floppy solid. Hemiacetal 8a (α/β 5:1) had R_{f} 0.3, 0.25 (cHex/EtOAc 7:3). HRMS (ESI⁺): m/z [M+Na]⁺ calcd for C₃₆H₄₅Cl₆N₅O₁₀SiNa, 968.0964; found 968.0972.

(Benzyl 3-O-benzyl-4-O-tert-butyldimethylsilyl-2-deoxy-2-trichloroacetamido-α-L-altropyranosyluronate)-(1→3)-4-azido-2-trichloroacetamido-2,4,6-trideoxy-β-D-galactopyranosyl (N-phenyl)trifluoroacetimidate (9a) and 2-trichloromethyl [(benzyl 3-O-benzyl-2-deoxy-4-O-tert-butyldimethylsilyl-2-trichloroacetamido-α-L-altropyranosyluronate)-(1→3)-4-azido-1,2,4,6-tetradeoxy-α-D-galactopyrano]-[2,1,d]-oxazoline (10a). PTFACl (970 μL, 6.1 mmol, 2.0 equiv.) and Cs₂CO₃ (1.19 g, 3.67 mmol, 1.2 equiv.) were added to hemiacetal 8a (2.9 g, 3.06 mmol, 1.0 equiv.) in acetone (34 mL). After stirring for 1 h at rt, the reaction mixture was filtered through a pad of Celite and washed with DCM (50 mL) twice. The filtrate was concentrated under reduced pressure and the residue was purified by flash chromatography (cHex/Et₃N 99:1 for column equilibration, then cHex/EtOAc 100:0→90:10) to give the expected donor as a 6:4 mix of PTFA 9a and oxazoline 10a (3.09 g, 91%). The PTFA donor 9a had R_{f} 0.4 (cHex/EtOAc 85:15).

Oxazoline 10a had R_{f} 0.45 (cHex/EtOAc 85:15). HRMS (ESI⁺): m/z [M+H]⁺ calcd for C₃₆H₄₄Cl₆N₅O₉Si, 928.1039; found 928.1004.

Oliomerization from Building Block 6a (4_{A}-TBS)

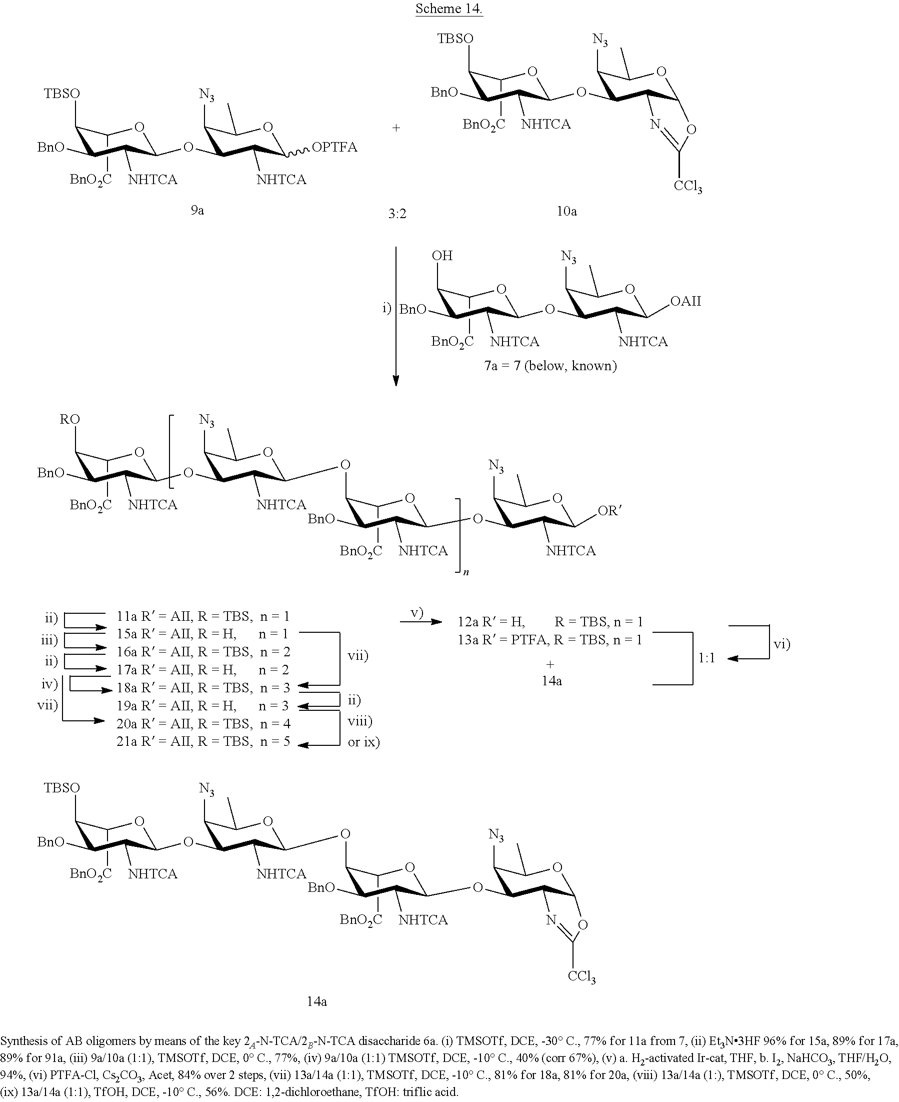

Allyl (benzyl 3-O-benzyl-2-deoxy-4-O-tert-butyldimethylsilyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (11a). Freshly activated MS 4 Å (2.63 g) was added to a mix of donors 9a and 10a (3.08 g, 2.75 mmol, 1.1 equiv. theo.) and disaccharide acceptor 7a (2.19 g, 2.51 mmol, 1.0 equiv.) in anhyd. DCE (52 mL) and the suspension was stirred for 30 min under an Ar atmosphere at rt. After cooling to −30° C., TMSOTf (45 μL, 250 μmol, 0.1 equiv.) was added slowly and stirring went on for 40 min at this temperature. A TLC analysis (Tol/EtOAc 8:2) showed some remaining acceptor 7a in minor amount and the presence of a new spot (R_{f} 0.5). Et₃N (10 μL, 1.0 equiv.) was added. The suspension was filtered through a fitted funnel and washed with DCM (50 mL) twice. Volatiles were evaporated and the residue was purified by flash chromatography (Tol/EtOAc 95:5→90:10) to give tetrasaccharide 11a as a white solid (3.48 g, 1.93 mmol, 77%). The coupling product 11a had R_{f} 0.35 (Tol/EtOAc 9:1). HRMS (ESI⁺): m/z [M+Na]⁺ calcd for C₆₉H₇₈Cl₁₂N₁₀O₁₉SiNa, 1821.1375; found 1821.1779.

(Benzyl 3-O-benzyl-2-deoxy-4-O-tert-butyldimethylsilyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranose (12a). [Ir(COD)(PMePh₂)₂]PF₆ (9.4 mg, 11 nmol, 0.02 equiv.) was dissolved in anhyd. THF (1.1 mL) and stirred for 20 min under an H₂ atmosphere. The resulting yellow solution was degassed repeatedly with Ar and transferred by means of a cannula into a solution of allyl glycoside 11a (1.0 g, 554 nmol, 1.0 equiv.) in anhyd. THF (2.8 mL). The reaction mixture was stirred for 2 h at rt, at which time a solution of I₂ (281 mg, 1.11 mmol, 2.0 equiv.) in THF/H₂O (4:1, 3.3 mL) was added. After stirring for 1 h at rt, a TLC analysis (Tol/EtOAc 8:2) revealed the full consumption of the isomerization product (R_{f} 0.5) and the presence of two more polar spots (R_{f} 0.3, 0.1). 10% Aq. Na₂SO₃ (50 mL, 20 equiv.) was added and volatiles were evaporated. The aq. phase was extracted with DCM (60 mL) thrice. The combined organic layers were washed with water and brine, dried over Na₂SO₄, filtered, and concentrated under vacuum. Purification of the residue by flash chromatography (cHex/EtOAc 80:20→60:40) gave the expected hemiacetal 12a (916 mg, 94%) as a white floppy solid. Hemiacetal 12a (α/β 5:1) had R_{f} 0.3, 0.1 (Tol/EtOAc 8:2). HRMS (ESI⁺): m/z [M+Na]⁺ calcd for C₆₆H₇₄Cl₁₂N₁₀O₁₉SiNa, 1781.1061; found 1781.1320.

(Benzyl 3-O-benzyl-2-deoxy-4-O-tert-butyldimethylsilyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl (N-phenyl)trifluoroacetimidate (13a). [Ir(COD)(PMePh₂)₂]PF₆ (32 mg, 38 nmol, 0.04 equiv.) was dissolved in anhyd. THF (3.9 mL) and stirred for 20 min under an H₂ atmosphere. The resulting yellow solution was degassed repeatedly with Ar and transferred by means of a cannula into a solution of allyl glycoside 11a (1.72 g, 953 nmol, 1.0 equiv.) in anhyd. THF (4.5 mL). The reaction mixture was stirred for 2 h at rt, at which time a solution of 12 (484 mg, 1.9 mmol, 2.0 equiv.) in THF/H₂O (4:1, 5.7 mL) was added. After stirring for 1.5 h at rt, a TLC analysis (Tol/EtOAc 8:2) revealed the full consumption of the isomerization product (R_{f} 0.5) and the presence of two more polar spots (R_{f} 0.3, 0.1). 10% Aq. Na₂SO₃ (50 mL, 20 equiv.) was added and volatiles were evaporated. The aq. phase was extracted with DCM (200 mL) twice. The combined organic layers were washed with water and brine, dried over Na₂SO₄, filtered, and concentrated under vacuum. Flash chromatography (cHex/EtOAc 80:20→40:60) f the residue gave the expected hemiacetal 12a (1.66 g, 99%) as a white floppy solid.

PTFACl (287 μL, 1.81 mmol, 2.0 equiv.) and Cs₂CO₃ (355 mg, 1.09 mmol, 1.2 equiv.) were added to hemiacetal 12a (1.6 g, 910 μmol, 1.0 equiv.) in acetone (10 mL). After stirring for 1.5 h at rt, a TLC (cHex/EtOAc 7:3) follow up revealed that conversion was complete. The reaction mixture was filtered through a pad of Celite and washed with DCM (50 mL) twice. The filtrate was concentrated under reduced pressure and the residue was purified by flash chromatography (cHex/EtOAc 100:0→70:30) to give the expected donor as a mix of PTFA 13a and oxazoline 14a (1.43 g, 84% over two steps). The PTFA donor 13a had R_{f} 0.55 (cHex/EtOAc 7:3).

Oxazoline 14a had R_{f} 0.65 (cHex/EtOAc 7:3). HRMS (ESI⁺): m/z [M+NH₄]⁺ calcd for C₆₆H₇₆Cl₁₂N₁₁O₁₈Si, 1758.1401; found 1758.1414.

Allyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (15a). Et₃N·3HF (1.25 mL, 7.67 mmol, 8.0 equiv.) was added to a solution of the fully protected tetrasaccharide 11a (1.73 g, 958 μmol, 1.0 equiv.) in anhyd. THF (3.45 mL), and the solution was stirred at rt for 48 h at which time a TLC follow up (Tol/EtOAc 8:2) showed reaction completion. MeOH (1.0 mL) was added and volatiles were eliminated under vacuum. Flash chromatography (Tol/EtOAc 80:20→75:25, then Tol/Acet 50:50→20:80) of the residue gave the desired alcohol 15a (1.55 g, 96%). The tetrasaccharide acceptor 15a had R_{f} 0.45 (Tol/EtOAc 7:3). HRMS (ESI⁺): m/z [M+H+K]²⁺ calcd for C₆₃H₆₃Cl₁₂N₁₀O₁₉K, 862.0164; found 862.0118.

Allyl (benzyl 3-O-benzyl-2-deoxy-4-O-tert-butyldimethylsilyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (16a). Freshly activated MS 4 Å (332 mg) was added to a mix of donors 9a and 10a (414 mg, 370 μmol, 1.25 equiv. theo.) and tetrasaccharide acceptor 15a (500 mg, 296 μmol, 1.0 equiv.) in anhyd. DCE (6.7 mL) and the suspension was stirred for 30 min under an Ar atmosphere at rt. After cooling to 0° C. and stirring for 10 min, TMSOTf (6.6 μL, 30 μmol, 0.1 equiv., 20 μL from a TMSOTf/DCE solution (1:2.7 v/v)) was added and stirring went on for 45 min at 0° C. A TLC analysis (Tol/EtOAc 8:2) showed some remaining acceptor 15a in minor amount (R_{f} 0.15) and the presence of a new spot (R_{f} 0.35). After stirring for an additional 15 min, Et₃N (50 μL of a Et₃N/DCE solution (1:2 v/v)) was added. The suspension was filtered through a fitted funnel and washed with DCM (50 mL) twice. Volatiles were evaporated and the residue was purified by flash chromatography (Tol/EtOAc 90:10→70:30) to give hexasaccharide 16a as a white solid (599 mg, 223 μmol, 77%). HRMS (MALDI): m/z [M+Na]⁺ calcd for C₉₉H₁₀₇C18N₁₅O₂₈SiNa, 2635.1499; found 2635.0943. HRMS (MALDI): m/z [M+K]⁺ calcd for C₉₉H₁₀₇Cl₁₈N₁₅O₂₈SiK 2652.1191; found 2652.0420.

Allyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (17a). Et₃N·3HF (103 μL, 635 μmol, 8.0 equiv.) was added to a solution of the fully protected hexasaccharide 16a (208 mg, 79 μmol, 1.0 equiv.) in anhyd. THF (290 μL), and the solution was stirred at rt for 48 h at which time a TLC follow up (Tol/EtOAc 8:2) showed reaction completion. MeOH (1.0 mL) was added and volatiles were eliminated under vacuum. Flash chromatography (Tol/EtOAc 80:20→50:50) of the residue gave the desired alcohol 17a (176 mg, 89%). The hexasaccharide acceptor 17a had R_{f} 0.35 (Tol/EtOAc 7:3). HRMS (ESI⁺): m/z [M+NH₄]⁺ calcd for C₉₃H₉₇Cl₁₈N₁₆O₂₈ 2517.1033; found 2516.7451. HRMS (ESI⁺): m/z [M+2NH₄]²⁺ calcd for C₉₃H₉₇Cl₁₈N₁₆O₂₈ 1267.5687; found 1267.5885.

Allyl (benzyl 3-O-benzyl-2-deoxy-4-O-tert-butyldimethylsilyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (18a). [2+6 glycosylation] Freshly activated MS 4 Å (13 mg) was added to a mix of donors 9a and 10a (17 mg, 15 μmol, 1.25 equiv.) and hexasaccharide acceptor 17a (30 mg, 12 μmol, 1.0 equiv.) in anhyd. DCE (2.5 mL) and the suspension was stirred for 30 min under an Ar atmosphere at rt. After cooling to 0° C. and stirring for 15 min, TMSOTf (0.2 μL, 1.2 μmol, 0.1 equiv., 10 μL of a TMSOTf/DCE solution (1:49 v/v)) was added and stirring went on for 40 min at −10° C. More TMSOTf (0.1 equiv.) was added. After stirring for 20 min at −10° C., a TLC analysis (Tol/EtOAc 8:2) some remaining acceptor 17a and a new spot (R_{f} 0.35), albeit no further evolution. Et₃N (0.1 equiv., 10 μL, of a Et₃N/DCE solution (1:49 v/v)) was added. The suspension was filtered through a fitted funnel and washed with DCM (20 mL) twice. Volatiles were evaporated and the residue was purified by flash chromatography (Tol/EtOAc 90:10→70:30) to give octasaccharide 18a as a white solid (16.5 mg, 47 μmol, 40%) and some remaining hexasaccharide acceptor 17a (12 mg, 40%).

[4+4 glycosylation] Freshly activated MS 4 Å (124 mg) was added to a mix of donors 13a and 14a (126 mg, 65 μmol, 1.1 equiv.) and tetrasaccharide acceptor 15a (100 mg, 59 μmol, 1.0 equiv.) in anhyd. DCE (2.5 mL) and the suspension was stirred for 45 min under an Ar atmosphere at rt. After cooling to −10° C. and stirring for 15 min, TMSOTf (0.3 μL, 2 μmol, 0.03 equiv., 15 μL of a TMSOTf/DCE solution (1:49 v/v)) was added and stirring went on for 20 min at −10° C. A TLC analysis (Tol/EtOAc 8:2) showed the presence of acceptor 15a in minor amount and a new spot (R_{f} 0.35). After stirring for an additional 15 min, Et₃N (20 μL of a Et₃N/DCE solution (1:36.5 v/v)) was added. The suspension was filtered through a fitted funnel and washed with DCM (50 mL) twice. Volatiles were evaporated and the residue was purified by flash chromatography (Tol/EtOAc 95:5→70:30) to give octasaccharide 18a as a white solid (163 mg, 47 μmol, 81%). The coupling product 18a had R_{f} 0.5 (Tol/EtOAc 4:1). HRMS (ESI⁺): m/z [M+2NH₄]²⁺ calcd for C₁₂₉H₁₄₂Cl₂₄N₂₂O₃₇Si, 1732.6169; found 1732.6240.

Allyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (19a). Et₃N·3HF (75 μL, 455 μmol, 5.0 equiv.) was added to a solution of the fully protected octasaccharide 18a (312 mg, 91 μmol, 1.0 equiv.) in anhyd. THF (455 μL), and the solution was stirred at rt for 52 h at which time a TLC follow up (Tol/EtOAc 8:2) showed that only minor traces of the starting 18a and the presence of a major more polar product. MeOH (300 μL) was added and after 10 min at rt, volatiles were eliminated under vacuum. The residue was taken in DCM (100 mL) and the organic phase was washed with satd aq. NaHCO₃ (30 mL) and brine (30 mL), dried on Na₂SO₄, and filtered. After concentration under vacuum, flash chromatography (Tol/EtOAc 80:20→50:50) of the residue gave the desired alcohol 19a (269 mg, 81 μmol, 89%). The octasaccharide acceptor 19a had R_{f} 0.3 (Tol/EtOAc 7:3). HRMS (ESI⁺): m/z [M+2NH₄]²⁺ calcd for C₁₂₃H₁₂₈Cl₂₄N₂₂O₃₇Si, 1675.5724; found 1676.5715.

Allyl (benzyl 3-O-benzyl-2-deoxy-4-O-tert-butyldimethylsilyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (20a). Freshly activated MS 4 Å (227 mg) was added to a mix of donors 13a and 14a (139 mg, 72 μmol, 1.2 equiv.) and hexasaccharide acceptor 17a (150 mg, 60 μmol, 1.0 equiv.) in anhyd. DCE (4.5 mL) and the suspension was stirred for 1 h under an Ar atmosphere at rt. After cooling to −10° C. and stirring for 15 min, TMSOTf (0.3 μL, 2 μmol, 0.03 equiv., 15 μL of a TfOH/DCE solution (1:49 v/v)) was added and stirring went on for 20 min at −10° C. A TLC analysis (Tol/EtOAc 8:2) showed the presence of a new spot (R_{f} 0.35) and stirring went on for another 20 min at −10° C. Et₃N (30 μL of a Et₃N/DCE solution (1:42 v/v)) was added. Solids were filtered and washed with DCM (20 mL) twice. Volatiles were evaporated and the residue was purified by flash chromatography (Tol/EtOAc 95:5→70:30) to give decasaccharide 20a as a white solid (206 mg, 48 μmol, 81%) in addition to contaminated fractions. The fully protected decasaccharide 20a had R_{f} 0.5 (Tol/EtOAc 7:3). HRMS (ESI⁺): m/z [M+2NH₄]²⁺ calcd for C₁₅₉H₁₇₁Cl₃₀N₂₇O₄₆Si, 1675.5724; found 1676.5715.

Allyl (benzyl 3-O-benzyl-2-deoxy-4-O-tert-butyldimethylsilyl-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (21a). Route 1. Freshly activated MS 4 Å (137 mg) was added to a mix of donors 13a and 14a (50 mg, 26 μmol, 1.3 equiv.) and octasaccharide acceptor 19a (66 mg, 20 μmol, 1.0 equiv.) in anhyd. DCE (2.7 mL) and the suspension was stirred for 30 min under an Ar atmosphere at rt. After cooling to 0° C. and stirring for 10 min, TMSOTf (0.14 μL, 1 μmol, 0.03 equiv., 10 μL of a TfOH/DCE solution (1:70 v/v)) was added and stirring went on for 1 h at 0° C. A TLC analysis (Tol/EtOAc 8:2) showed the presence of a new spot (R_{f} 0.35). Et₃N (80 μL of a solution in DCE (1:35 v/v)) was added. Solids were filtered and washed with DCM (20 mL) twice. Volatiles were evaporated and the residue was purified by flash chromatography (Tol/EtOAc 90:10→70:30) to give dodecasaccharide 21a as a white solid (51 mg, 10 μmol, 50%) in addition to contaminated fractions and a less polar compound corresponding to the silylated acceptor (9 mg, 2.6 μmol, 13%). The silylated side-product had R_{f} 0.65 (Tol/EtOAc 75:25). HRMS (ESI⁺): m/z [M+2NH₄]²⁺ calcd for C₁₂₆H₁₃₈C₁₂₄N₂₂O₃₇Si, 1712.0916; found 1712.0918.

Route 2. Freshly activated MS 4 Å (310 mg) was added to a mix of donors 13a and 14a (57 mg, 29 μmol, 1.3 equiv.) and octasaccharide acceptor 19a (75 mg, 23 μmol, 1.0 equiv.) in anhyd. DCE (3.1 mL) and the suspension was stirred for 1 h under an Ar atmosphere at rt. After cooling to −10° C. and stirring for 5 min, TfOH (0.1 μL, 1 μmol, 0.03 equiv., 10 μL of a TfOH/DCE solution (1:125 v/v)) was added and stirring went on for 25 min at −10° C. A TLC analysis (Tol/EtOAc 8:2) showed the presence of acceptor 19a in minor amount and a new spot (R_{f} 0.35). After stirring for an additional 35 min at −10° C., Et₃N (40 μL of a Et₃N/DCE solution (1:50 v/v)) was added. The suspension was filtered through a fitted funnel and washed with DCM (50 mL) twice. Volatiles were evaporated and the residue was purified by flash chromatography (Tol/EtOAc 90:10→70:30) to give dodecasaccharide 21a as a white solid (66 mg, 13 μmol, 56%) in addition to contaminated fractions. The coupling product 21a had R_{f} 0.3 (Tol/EtOAc 75:25). HRMS (ESI⁺): m/z [M+2NH₄]²⁺ calcd for C189H₂₀₀Cl₃₆N₃₂O₅₅Si, 2550.1213; found 2550.0933.

### Example 6: Strategy 2_{A}-NTCA,2_{B}-NTCA, 4_{A}-Nap

The Ready-for-Oligomerization 4_{A}-Nap AB Building Block

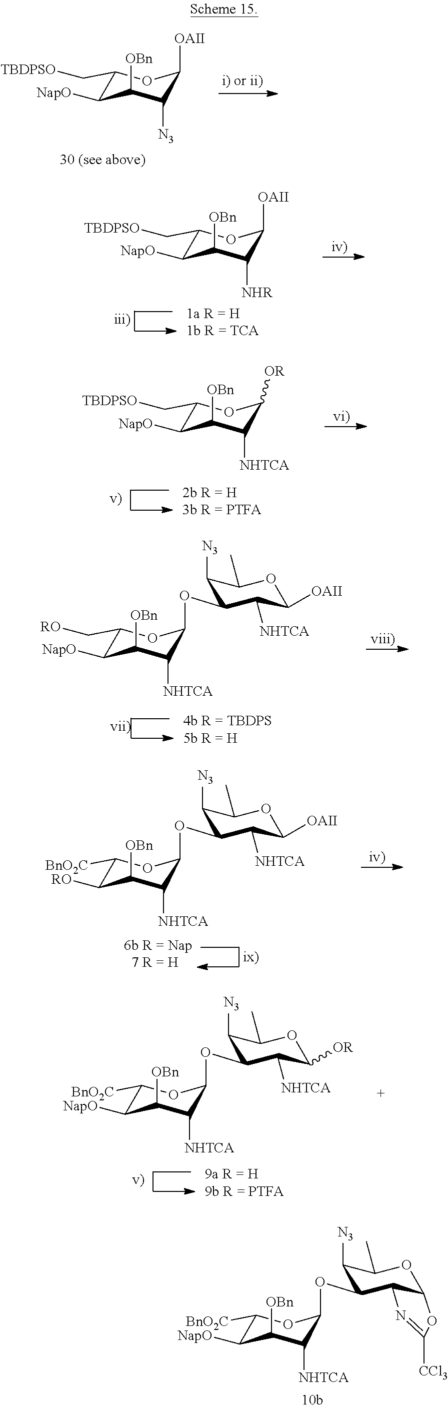

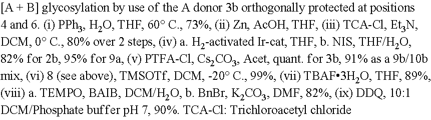

Allyl 2-amino-3-O-benzyl-6-O-tert-butyldiphenylsilyl-2-deoxy-4-O-(2-naphthylmethyl)-α-L-altropyranoside (1a). A solution of the azido precursor 30 (1.0 g, 1.4 mmol) in THF (7.0 mL) was treated with PPh₃ (0.4 g, 1.5 mmol, 1.1 equiv.) and H₂O (0.5 mL, 28 mmol, 20.0 equiv.). The reaction mixture was heated to 60° C. and stirred overnight. Follow up by TLC (Tol/EtOAc 9:1) indicated the total conversion of the starting material (R_{f} 0.7) and the presence of a more polar product (R_{f} 0.1). The reaction mixture was concentrated and coevaporated repeatedly with toluene under reduced pressure. The crude material was purified by flash chromatography (Tol/EtOAc/NH₃OH 85:15:2) to give amine 1a (0.73 g, 73%) as a colorless oil. ¹H NMR (CDCl₃) δ 7.88-7.17 (m, 22H, H_{Ar}), 6.02-5.91 (m, 1H, CH═CH₂), 5.31 (dq, 1H, CH═CH₂), 5.19 (dq, 1H, CH═CH₂), 4.79 (d, 2H, CH_{2Nap}), 4.58 (d, 1H, J_{1,2}=5.1 Hz, H-1), 4.54 (dd, 2H, CH_{2Bn}), 4.34 (dd, 1H, J_{5,6}=5.5 Hz, J_{5,4}=10.4 Hz, H-5), 4.33 (ddt, 1H, CH_{2All}), 4.05 (ddt, 1H, CH_{2All}), 4.04-4.01 (m, 1H, H-4), 3.89 (dd, 1H, J_{6b,5}=4.9 Hz, J_{6b,6a}=10.6 Hz, H-6b), 3.84 (dd, 1H, J_{6a,5}=5.0 Hz, J_{6a,6b}=10.6 Hz, H-6a), 3.66 (dd, 1H, J_{3,4}=3.3 Hz, J_{3,2}=8.2 Hz, H-3), 3.45 (dd, 1H, J_{2,1}=5.2 Hz, J_{2,3}=8.0 Hz, H-2), 1.08 (s, 9H, H-^{t}Bu_{TBDPS}). ¹³C NMR (CDCl₃) δ 135.9 (C_{q,Nap}), 135.7 (C_{qPh,TBDPS}), 135.6 (C_{qPh,TBDPS}), 134.9 (C_{qBn}), 134.4 (CH═CH₂), 133.9 (C_{q,Nap}), 133.7 (C_{q,Nap}), 129.8-125.3 (C_{Ar,Bn,Ph,Nap}), 117.1 (CH═CH₂), 101.4 (C-1_{A}, ¹J_{C,H}=163.7 Hz), 78.0 (C-3), 73.0 (C-5), 71.7 (CH_{2Nap}), 71.6 (CH_{2Bn}), 71.0 (C-4), 68.9 (CH_{2All}), 63.3 (C-6), 51.9 (C-2), 26.8 (CH_{3tBu, TBDPS}), 19.3 (C_{qtBu,TBDPS}).

Allyl 3-O-benzyl-6-O-tert-butyldiphenylsilyl-2-deoxy-4-O-(2-naphthylmethyl)-2-trichloroacetamido-α-L-altropyranoside (1b). Zn (24.7 g, 378 mmol, 8.0 equiv.) and AcOH (21.6 mL, 378 mmol, 10 equiv.) was added to the azido precursor 30 in anhyd. THF (470 mL). After stirring for 1 h, a TLC analysis (Tol/EtOAc 10:1) showed the absence of azide 1 (R_{f} 0.85) and the presence of a more polar spot. The suspension was filtered over a pad of Celite and washed with DCM. The DCM layer was washed with satd aq. NaHCO₃, water, and brine, dried over Na₂SO₄, concentrated under reduced pressure, and dried under high vacuum.

The crude amine 1a was dissolved in DCM (100 mL) and cooled to 0° C. Et₃N (9.8 mL, 70.8 mmol, 1.5 equiv.) was added followed by the dropwise addition of trichloroacetyl chloride (6.85 mL, 61.4 mmol, 1.3 equiv.) while maintaining the temperature at 0° C. After stirring for 1 h, a TLC follow up (Tol/EtOAc) indicated reaction completion. The reaction was quenched by addition of methanol (2.0 mL). Following dilution with DCM (200 mL), washing with 50% brine, the DCM layer was separated, dried and concentrated. Flash chromatography of the crude (cHex/EtOAc 54:6) gave trichloroacetamide 1b as a colorless oil (31.6 g, 38.0 mmol, 80%). Allyl glycoside 1b had R_{f} 0.2 (Tol/EtOAc 7:3). ¹H NMR (CDCl₃) δ 7.85-7.83 (m, 1H, H_{Ar}), 7.77-7.72 (m, 6H, H_{Ar}), 7.63 (brs, 1H, H_{Ar}), 7.52-7.28 (m, 14H, H_{Ar}), 6.75 (d, 1H, J_{2,NH}=8.4 Hz, NH), 5.97-5.89 (m, 1H, CH_{All}), 5.37-5.32 (m, 1H, CH_{2All}), 5.24-5.20 (m, 1H, CH_{2All}), 4.92 (d, 1H, J=12.0 Hz, CH_{2Nap}), 4.88 (dₚₒ, 1H, J_{1,2}=4.8 Hz, H-1), 4.73 (d, 2H, J=12.0 Hz, CH_{2Bn}), 4.72 (dₚₒ, 1H, CH_{2Nap}), 4.61 (d, 1H, CH_{2Bn}), 4.51-4.48 (m, 1H, CH_{2All}), 4.51 (ddd, 1H, J_{2,3}=1.0 Hz, H-2), 4.37 (dt, J_{5,6b}=2.8 Hz, H-5), 4.29-4.24 (m, 1H, CH_{2All}), 4.14 (dd, 1H, J_{5,6a}=3.2 Hz, J_{6a,6b}=11.2 Hz, H-6a), 4.06-3.97 (m, 3H, H-3, H-6b, CH_{2All}), 3.93 (dd, J_{3,4}=3.2 Hz, J_{4,5}=9.2 Hz, H-4), 1.00 (s, 9H, CH_{3, TBDPS}). ¹³C NMR (CDCl₃) δ 161.2 (CO_{NHTCA}), 137.9, 133.2 (2C), 133.0, 129.0, 125.3 (C_{q,Ar}), 133.6 (CH_{All}), 135.8, 135.5, 129.7, 128.3, 128.2, 127.9, 127.7 (2C), 127.6, 126.7, 126.1, 125.9, 125.8 (22C, C_{Ar}), 117.3 (CH_{2All}), 97.3 (C-1_{A}, ¹J_{C,H}=169 Hz), 92.1 (CCl₃), 72.0 (C-3), 71.5 (CH_{2Nap}), 70.9 (CH_{2Bn}), 70.5 (C-4), 68.6 (C-5), 68.3 (CH_{2All}), 62.8 (C-6), 50.8 (C-2), 26.9 (CH_{3, TBDPS}), 19.4 (C_{TBDPS}). HRMS (ESI⁺): m/z [M+NH₄]⁺ calcd for C₄₅H₅₂Cl₃N₂O₆Si, 849.2655; found 849.2657.

3-O-Benzyl-2-deoxy-4-O-(2-napthylmethyl)-6-O-tert-butyldiphenylsilyl-2-trichloroacetamido-α/β-L-altropyranose (2b). [Ir(COD)(PMePh₂)₂]PF₆ (965 mg, 1.14 mmol, 0.03 equiv.) was dissolved in anhyd. THF (100 mL) and stirred for 30 min under an H₂ atmosphere. The resulting yellow solution was degassed repeatedly with Ar and transferred by means of a cannula into a solution of allyl glycoside 1b (31.6 g, 38.0 mmol, 1.0 equiv.) in anhyd. THF (200 mL). The reaction mixture was stirred for 4 h at rt, at which time a solution of NIS (11.2 g, 49.4 mmol, 1.3 equiv.) in H₂O (60 mL) was added. After stirring for 1 h at rt, a TLC analysis (Tol/EtOAc 9:1) revealed the full consumption of the isomerization product (R_{f} 0.7) and the presence of a more polar spot (R_{f} 0.4). 10% Aq. Na₂SO₃ was added and volatiles were evaporated. The aq. phase was extracted with DCM (200 mL) twice. The combined organic layers were washed with water and brine, dried over Na₂SO₄, filtered, and concentrated under vacuum. Purification of the residue by flash chromatography (cHex/EtOAc 90:10→88:12) yielded the expected hemiacetal 2b (24.6 g, 31.0 mmol, 82%) as a white floppy solid. Hemiacetal 2b (α/β 5:1) had R_{f} 0.5 (cHex/EtOAc 4:1). ¹H NMR (CDCl₃) δ 7.85-7.64 (m, 10.4H, H_{Ar}), 7.52-7.28 (m, 18.6H, H_{Ar}), 7.04 (d, 1H, J_{2,NH}=5.6 Hz, NH_{α}), 6.80 (d, 0.24H, J_{2,NH}=5.6 Hz, NH_{β}), 5.64 (d, 0.25H, J_{1,OH}=11.2 Hz, OH_{β}), 5.41 (ddₚₒ, 1H, J_{1,2}=3.6 Hz, H-1_{α}), 5.10 (d, 0.25H, J_{1,2}=1.0 Hz, H-1_{β}), 4.90 (d, 0.29H, J=11.2 Hz, CH_{2Nap,β}), 4.82-4.74 (m, 2.35H, CH_{2Nap,α}, CH_{2Nap,β}), 4.69 (dₚₒ, 1.5H, CH_{2Bn,α}, CH_{2Bn,β}), 4.61 (d, 1H, J=11.6 Hz, CH_{2n,α}), 4.39 (ddd, 0.29H, J_{2,3}=3.6 Hz, H-2β), 4.30-4.24 (brm, 2.56H, H-2_{α}, H-3_{β}, H-5_{α}, H-5β), 4.20 (dd, 0.27H, J_{5,6b}=2.0 hz, J_{6a,6b}=11.2 Hz, H-6a_{β}), 4.11-3.90 (m, 4.71H, H-3_{α}, H-4_{α}, H-4_{β}, H-6a_{α}, H—H-6b_{α}, H-6b_{β}), 2.98 (d, 1H, J_{1,OH}=4.0 Hz, OH_{α}), 1.12 (s, 2.22H, CH_{3, TBDPS}), 1.09 (s, 9H, CH_{3, TBDPS}). ¹³C NMR (CDCl₃) δ 162.1 (CO_{NHTCA,α}), 161.4 (CO_{NHTCA,β}), 137.8, 136.5, 134.9, 134.8, 133.7, 133.4, 133.1 (2C), 133.0, 132.9 (C_{q,Ar}), 135.8, 135.7, 135.5 (2C), 129.7 (3C), 128.7, 128.4, 128.3, 128.2, 128.0, 127.9 (2C), 127.7 (3C), 127.6 (2C), 126.6, 126.2, 126.1, 125.9, 125.7, 125.6 (C_{Ar}), 93.4 (C-1_{β}, ¹J_{C,H}=175 Hz), 91.3 (C-1_{A}, ¹J_{C,H}=169 Hz), 92.2, 91.8 (CCl₃), 74.3 (CH_{2Nap,β}), 74.2 (C-3_{α}, C-3β), 73.9 (C-5_{β}), 72.9 (CH_{2Nap,α}), 72.1 (CH_{2Bn,β}), 72.1 (C-5_{α}), 71.7 (CH_{2n,α}), 71.3 (C-4_{α}), 70.8 (C-4_{β}), 67.7 (C-5_{β}), 63.0 (C-6_{α}), 62.5 (C-6_{β}), 53.1 (C-2_{α}), 51.3 (C-2_{β}), 27.0, 26.9 (C_{TBDPS}), 19.4 (CH_{3,TBDPS}). HRMS (ESI⁺): m/z [M+NH₄]⁺ calcd for C₄₄H₄₆Cl₃N₂O₆Si, 809.2342; found 809.2346.

3-O-Benzyl-2-deoxy-4-O-(2-napthylmethyl)-6-O-tert-butyldiphenylsilyl-2-trichloroacetamido-α/β-L-altropyranosyl N-(phenyl)trifluoroacetimidate (3b). PTFACl (7.74 mL, 37.3 mmol, 1.2 equiv.) and Cs₂CO₃ (11.1 g, 34.2 mmol, 1.1 equiv.) were added to hemiacetal 2b (24.6 g, 31.0 mmol, 1.0 equiv.) in acetone (300 mL). After stirring for 2 h at rt, the reaction mixture was filtered through a pad of Celite and washed with DCM (50 mL) twice. The filtrate was concentrated under reduced pressure and dried under vacuum to give the crude donor 3b (˜30.0 g, 31.0 mmol, quant.), which was used as such in the next step. The PTFA donor 3b had R_{f} 0.92 (Tol/EtOAc 9:1). HRMS (ESI⁺): m/z [M+Na]⁺ calcd for C₅₀H₄₈Cl₃F₃N₂O₆SiNa, 985.2197; found 985.2191.

Allyl (3-O-benzyl-6-O-tert-butyldiphenylsilyl-2-deoxy-4-O-(2-naphthylmethyl)-2-trichloroacetamido-α-L-altropyranosyl)-(1→3)-4-azido-2-trichloroacetamido-2,4,6-trideoxy-β-D-galactopyranoside (4b). A mix of the crude PTFA donor 3b (˜30.0 g, 31.0 mmol, 1.0 equiv. theo.) and acceptor 8 (10.8 g, 32.0 mmol, 1.03 equiv.) were co-evaporated with anhyd. toluene (50 mL) and then dried under high vacuum. Freshly activated MS 4 Å (15.0 g) was added to a solution of the starting materials in anhyd. DCM (520 mL) and the suspension was stirred for 1 h under an Ar atmosphere at rt. After cooling to −15° C., TMSOTf (281 μL, 0.05 equiv.) was added slowly and stirring went on for 40 min during which the bath temperature was kept at −15° C. A TLC analysis (Tol/EtOAc 9:1) showed the absence of donor 3b and the presence of a new spot (R_{f} 0.6) in addition to a slight amount of acceptor 8 (R_{f} 0.05). At completion, Et₃N (300 μL) was added. The suspension was filtered through a fitted funnel and washed with DCM (100 mL) twice. Volatiles were evaporated and the residue was purified by flash chromatography (cHex/EtOAc 90:10→85:15) to give disaccharide 4b as a white solid (35.6 g, 31.0 mmol, 99%). The coupling product 4b had ¹H NMR (CDCl₃) δ 7.82-7.80 (m, 1H, H_{Ar}), 7.74-7.68 (m, 6H, H_{Ar}), 7.61 (brs, 1H, H_{Ar}), 7.54-7.28 (m, 14H, H_{Ar}), 6.75 (d, 1H, J_{2,NH}=6.8 Hz, NH_{B}), 6.49 (d, 1H, J_{2,NH}=8.4 Hz, NH_{A}), 5.91-5.81 (m, 1H, CH_{All}), 5.30-5.25 (m, 1H, CH_{2All}), 5.21-5.18 (m, 1H, CH_{2All}), 4.92 (d, 1H, J_{1,2}=8.4 Hz, H-1_{B}), 4.83-4.75 (m, 3H, H-1_{A}, CH_{2Nap}), 4.73 (d, 1H, J=12.4 Hz, CH_{2Bn}), 4.60 (d, 1H, CH_{2Bn}), 4.55-4.48 (mₚₒ, 2H, H-3_{B}, H-5_{A}), 4.37-4.31 (m, 2H, H-2_{A}, CH_{2All}), 4.09-3.97 (m, 3H, H-6ab_{A}, CH_{2All}), 3.92 (t, 1H, J_{2,3}=J_{3,4}=3.2 Hz, H-3_{A}), 3.78 (dd, 1H, J_{4,5}=9.6 Hz, H-4_{A}), 3.58-3.54 (m, 2H, H-5_{B}, H-4_{B}), 3.45 (dddₚₒ, 1H, H-2_{B}), 1.21 (d, 3H, 6.4 Hz, H-6_{B}), 1.10 (s, 9H, CH_{3,TBDPS}). ¹³C NMR (CDCl₃) δ 162.2, 161.0 (CO_{NHTCA}), 138.3, 134.8, 133.5, 133.3, 133.1 (C_{q,Ar}), 133.5 (CH_{All}), 135.8, 135.5, 129.8, 128.8, 128.3 (2C), 127.8, 127.7 (2C), 127.6, 126.9, 126.2, 126.0, 125.8 (22C, C_{Ar}), 117.8 (CH_{2All}), 100.5 (C-1_{A}, ¹J_{C,H}=169 Hz), 97.3 (C-1_{B}, ¹J_{C,H}=163 Hz), 92.1, 91.8 (2C, CCl₃), 76.2 (C-3_{B}), 72.6 (C-3_{A}), 72.1 (CH_{2Nap}), 70.8 (CH_{2Bn}), 70.1 (CH_{2All}), 70.1 (C-4_{A}), 69.7 (C-5_{B}), 69.4 (C-5_{A}), 65.5 (C-4_{B}), 63.2 (C-6_{A}), 55.9 (C-2_{B}), 51.2 (C-2_{A}), 27.0 (CH_{3,TBDPS}), 19.5 (C_{TBDPS}), 17.3 (C-6_{B}). HRMS (ESI⁺): m/z [M+NH₄]⁺ calcd for C₅₃H₆₁Cl₆N₆O₉Si, 1163.2395; found 1163.2402.

Allyl (3-O-benzyl-2-deoxy-4-O-(2-naphthylmethyl)-2-trichloroacetamido-α-L-altropyranosyl)-(1→3)-4-azido-2-trichloroacetamido-2,4,6-trideoxy-β-D-galactopyranoside (5b). TBAF·3H₂O (10.7 g, 34.1 mmol, 1.1 equiv.) was added to disaccharide 4b (35.6 g, 31.0 mmol, 1.0 equiv.) in THF (300 mL) and the reaction mixture was stirred at rt for 4 h. A TLC analysis (Tol/EtOAc 1:4) showed the consumption of the fully protected 4b (R_{f} 1.0) and the presence of a polar spot. Acetic acid (2.1 mL, 3.73 mmol, 1.2 equiv.) was added and after stirring for 10 min, volatiles were evaporated. The residue was purified by flash chromatography (cHex/EtOAc 40:60→0:100) to give alcohol 5b (25.2 g, 27.7 mmol, 89%) as a white solid. Disaccharide 5b had R_{f} 0.25 (Tol/EtOAc 1:1). ¹H NMR (CDCl₃) δ 7.83-7.73 (m, 3H, H_{Ar}), 7.60 (brs, 1H, H_{Ar}), 7.53-7.46 (m, 4H, H_{Ar}), 7.42-7.32 (m, 4H, H_{Ar}), 6.87 (d, 1H, J_{2,NH}=6.8 Hz, NH_{B}), 6.64 (d, 1H, J_{2,NH}=8.0 Hz, NH_{A}), 5.90-5.80 (m, 1H, CH_{All}), 5.29-5.23 (m, 1H, CH_{2All}), 5.20-5.17 (m, 1H, CH_{2All}), 4.93 (d, 1H, J_{1,2}=8.4 Hz, H-1_{B}), 4.88 (brs, 1H, H-1_{A}), 4.83 (dd, 2H, J=12.4 Hz, J=7.6 Hz, CH_{2Nap}), 4.72 (dd, 1H, J=3.6 Hz, J=10.2 Hz, H-3_{B}), 4.67 (d, 1H, J=12.4 Hz, CH_{2Bn}), 4.50-4.44 (mₚₒ, 2H, H-5_{A}, CH_{2Bn}), 4.39-4.31 (m, 2H, H-2_{A}, CH_{2All}), 4.08-4.03 (m, 1H, CH_{2All}), 3.93-3.89 (m, 2H, H-3_{A}, H-6a_{A}), 3.82-3.75 (m, 3H, H-4_{B}, H-5_{B}, H-6b_{A}), 3.53-3.44 (m, 2H, H-2_{B}, H-4_{A}), 1.40 (d, 3H, J_{5,6}=6.4 Hz, H-6_{B}). ¹³C NMR (CDCl₃) δ 162.3, 161.1 (CO_{NHTCA}), 138.1, 134.4, 133.4, 133.1, 133.0 (C_{q,Ar}), 133.4 (CH_{All}), 128.5, 128.4, 128.3, 127.8, 127.7, 127.2, 126.3, 126.2, 125.7 (12C, C_{Ar}), 118.0 (CH_{2All}), 100.1 (C-1_{A}, ¹J_{C,H}=169 Hz), 97.2 (C-1_{B}, ¹J_{C,H}=164 Hz), 92.3, 91.8 (2C, CCl₃), 75.3 (C-3_{B}), 72.6 (C-3_{A}), 71.9 (CH_{2Nap}), 70.5 (CH_{2Bn}), 70.4 (C-4_{A}), 70.2 (CH_{2All}), 69.7 (C-5_{B}), 69.0 (C-5_{A}), 65.6 (C-4_{B}), 62.2 (C-6_{A}), 56.2 (C-2_{B}), 51.4 (C-2_{A}), 17.4 (C-6_{B}). HRMS (ESI⁺): m/z [M+NH₄]⁺ calcd for C₃₇H₄₁Cl₆N₆O₉, 925.1217; found 925.1223.

Allyl (benzyl 3-O-benzyl-4-O-(2-naphthylmethyl)-2-deoxy-2-trichloroacetamido-α-L-altropyranosyluronate)-(1→3)-4-azido-2-trichloroacetamido-2,4,6-trideoxy-β-D-galactopyranoside (6b). TEMPO (1.29 g, 8.26 mmol, 0.3 equiv.) was added, followed by BAIB (18.6 g, 57.8 mmol, 2.5 equiv.), to a biphasic solution of alcohol 5b (25.0 g, 3.7 mmol, 1.0 equiv.) in DCM/H₂O (2:1, 690 mL). The biphasic mixture stirred vigorously for 6 h at rt, at which point a TLC analysis (Tol/EtOAc 1:4) revealed the absence of alcohol 5b (R_{f} 0.65) and the presence of a polar product. NaHCO₃ (9.7 g, 115 mmol, 4.2 equiv.) was added in 100 mL water. The DCM layer was separated, and the aq. phase was extracted with DCM (100 mL) twice. The combined organic phases were dried by passing through a phase separator filter and concentrated to dryness. The residue was dissolved in anhyd. DMF (250 mL). Benzyl bromide (4.25 mL, 35.8 mmol, 1.3 equiv.) and K₂CO₃ (4.95 g, 35.8 mmol, 1.3 equiv.) were added and the suspension was stirred at rt for 2 h. At completion, satd aq. NH₄Cl was added and the aq. layer was washed with DCM (400 mL) thrice. The organic phases were combined, washed with brine, dried over Na₂SO₄, filtered, and concentrated under reduced pressure. The residue was purified by flash chromatography (cHex/EtOAc 77:23→70:30) to give the desired benzyl ester 6b (23.0 g, 25.3 mmol, 82%) as a white-brown solid. Uronate 6b had R_{f} 0.7 (Tol/EtOAc 4:1). ¹H NMR (CDCl₃) δ 7.85-7.75 (m, 3H, HA), 7.69 (brs, 1H, HA), 7.51-7.47 (m, 2H, H_{Ar}), 7.41-7.76 (m, 1H, H_{Ar}), 6.77 (d, 1H, J_{2,NH}=7.2 Hz, NH_{B}), 6.64 (d, 1H, J_{2,NH}=7.6 Hz, NH_{A}), 5.90-5.80 (m, 1H, CH_{All}), 5.29-5.17 (m, 5H, H-1_{A}, CH_{2Bn-6}, CH_{2All}), 4.85 (d, 1H, J_{4,5}=5.2 Hz, H-5_{A}), 4.82 (d, 1H, J_{1,2}=8.4 Hz, H-1_{B}), 4.73 (brs, 2H, CH_{2Nap}), 4.62 (dd, 1H, J_{3,4}=4.0 Hz, J_{2,3}=10.2 Hz, H-3_{B}), 4.56 (d, 1H, J=12.0 Hz, CH_{2Bn}), 4.48 (d, 1H, CH_{2Bn}), 4.35-4.30 (m, 1H, CH_{2All}), 4.23-4.18 (m, 1H, H-2_{A}), 4.09-4.02 (m, 2H, H-4_{A}, CH_{2All}), 3.87-3.84 (m, 2H, H-4_{B}, H-3_{A}), 3.61-3.54 (m, 2H, H-2_{B}, H-5_{B}) 1.28 (d, 3H, J_{5,6}=6.0 Hz, H-6_{B}). ¹³C NMR (CDCl₃) δ 168.9 (C-6_{A}), 162.0, 161.6 (CO_{NHTCA}), 137.4, 135.0, 134.6, 133.1 (2C) (C_{q,Ar}), 133.5 (CH_{All}), 128.7, 128.6, 128.4, 128.3, 128.1, 127.9 (2C), 127.7, 127.0, 126.2, 126.1, 125.8 (12C, C_{Ar}), 117.9 (CH_{2All}), 98.9 (C-1_{A}, ¹J_{C,H}=169 Hz), 97.7 (C-1_{B}, ¹J_{C,H}=162 Hz), 92.3, 92.1 (2C, CCl₃), 75.9 (C-3_{B}), 73.4 (C-3_{A}), 72.1 (C-4_{A}), 72.0 (CH_{2Nap}), 71.9 (CH_{2Bn}), 71.6 (C-5_{A}), 70.1 (CH_{2All}), 69.4 (C-5_{B}), 67.5 (CH_{2Bn-6}), 65.2 (C-4_{B}), 55.5 (C-2_{B}), 53.1 (C-2_{A}), 17.3 (C-6_{B}). HRMS (ESI⁺): m/z [M+NH₄]⁺ calcd for C₄₄H₄₇Cl₆N₆O₁₀, 1029.1491; found 1029.1479.

Allyl (benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (7).^{[1]} Disaccharide 6b (11.0 g, 10.8 mmol, 1.0 equiv.) was dissolved in DCM (200 mL) and phosphate buffer pH 7 (20 mL) was added. The biphasic mixture was cooled to 0° C. and DDQ (7.4 g, 2.1 mmol, 3.0 equiv.) was added. The reaction was slowly allowed to reach rt and stirred for 6 h at this temperature. At completion, 10% aq. NaHCO₃ (200 mL) was added and the biphasic mixture was diluted with DCM (200 mL). The DCM layer was separated, washed with brine, dried over Na₂SO₄, filtered, and concentrated under reduced pressure. The residue was purified by flash chromatography (cHex/EtOAc 70:30→60:40) to give alcohol 7 (8.6 g, 8.50 mmol, 90%) as a white solid. Disaccharide 7 had R_{f} 0.45 (Tol/EtOAc, 7:3). ¹H NMR (CDCl₃) δ 7.42-7.25 (m, 10H, H_{Ar}), 6.75 (dₚₒ, 1H, J_{2,NH}=8.8 Hz, NH_{A}), 6.73 (dₚₒ, 1H, J_{2,NH}=7.6 Hz, NH_{B}), 5.89-5.80 (m, 1H, CH_{All}), 5.31-5.24 (m, 3H, CH_{2Bn-6}, CH_{2All}), 5.20-5.17 (m, 1H, CH_{2All}), 4.98 (d, 1H, J_{1,2}=3.2 Hz, H-1_{A}), 4.83 (d, 1H, J_{1,2}=8.4 Hz, H-1_{B}), 4.72 (m, 3H, H-5_{A}, CH_{2Bn}), 4.64 (dd, 1H, J_{3,4}=4.0 Hz, J_{2,3}=10.6 Hz, H-3_{B}), 4.35-4.27 (m, 1H, H-2_{A}, CH_{2All}), 4.15 (dt, 1H, J_{4,5}=7.6 Hz, H-4_{A}), 4.07-4.02 (m, 2H, CH_{2All}), 3.88 (dd, 1H, J_{2,3}=5.6 Hz, J_{3,4}=3.6 Hz, H-3_{A}), 3.79 (d, 1H, H-4_{B}), 3.64 (qₚₒ, 1H, H-5_{B}), 3.52-3.46 (m, 1H, H-2_{B}), 2.78 (d, 1H, J_{4,OH}=7.6 Hz, OH), 1.26 (d, 3H, J_{5,6}=6.4 Hz, H-6_{B}). ¹³C NMR (CDCl₃) δ 168.8 (C-6_{A}), 162.1, 161.6 (CO_{NHTCA}), 137.2, 135.0 (C_{q,Ar}), 133.5 (CH_{All}), 128.7 (2C), 128.6, 128.4, 128.2, 125.2 (10C, C_{Ar}), 118.0 (CH_{2All}), 99.5 (C-1_{A}, ¹J_{C,H}=169 Hz), 97.4 (C-1_{B}, ¹J_{C,H}=162 Hz), 92.1, 91.9 (2C, CCl₃), 76.3 (C-3_{B}), 74.8 (C-3_{A}), 72.4 (CH_{2Bn}), 71.1 (C-5_{A}), 70.1 (CH_{2All}), 69.5 (C-5_{B}), 67.5 (CH_{2Bn-6}), 65.5 (C-4_{A}), 65.5 (C-4_{B}), 55.7 (C-2_{B}), 51.4 (C-2_{A}), 17.2 (C-6_{B}). HRMS (ESI⁺): m/z [M+NH₄]⁺ calcd for C₃₃H₃₉Cl₆N₆O₁₀, 889.0853; found 889.0860.

(Benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranose (9a). [Ir(COD)(PMePh₂)₂]PF₆ (286 mg, 0.338 mmol, 0.03 equiv.) in anhyd. THF (20 mL) was degassed and stirred for 20 min under an H₂ atmosphere. The resulting yellow solution was degassed repeatedly with Ar and poured into a solution of allyl glycoside 6b (11.4 g, 11.2 mmol, 1.0 equiv.) in anhyd. THF (200 mL). After stirring for 2 h at rt, a TCL follow up (Tol/EtOAc 4:1) revealed that the starting 6b (R_{f} 0.6) had been converted to a closely migrating product (R_{f} 0.65). NIS (3.0 g, 13.5 mmol, 1.2 equiv.) and H₂O (20 mL) were added and after stirring for another 1 h at rt, 10% aq. Na₂SO₃ was added. The reaction mixture was concentrated and the aq. phase was extracted with DCM (200 mL) thrice. The combined organic layers were a washed with brine, dried over anhyd. Na₂SO₄, and concentrated under reduced pressure. The residue was purified by flash chromatography with cHex/EtOAc (70:30→60:40) to give the expected hemiacetal 9a (10.4 mg, 10.7 mmol, 95%) as a white solid. The α/β hemiacetal 9a had R_{f} 0.3, 0.6 (Tol/EtOAc 7:3). HRMS (ESI⁺): m/z [M+NH₄]⁺ calcd for C₄₁H₄₃Cl₆N₆O₁₀, 989.1166; found 989.1177. The α isomer had ¹H NMR (CDCl₃) δ 7.87-7.76 (m, 3H, H_{Ar}), 7.71 (brs, 1H, H_{Ar}), 7.53-7.17 (m, 13H, H_{Ar}), 7.06 (d, 1H, J_{2,NH}=8.4 Hz, NH_{B}), 6.86 (d, 1H, J_{2,NH}=7.4 Hz, NH_{A}), 5.40 (d, 1H, J_{1,2}=6.4 Hz, H-1_{A}), 5.28 (t, 1H, J_{1,2}=3.5 Hz, H-1_{B}), 5.19 (ddₚₒ, 2H, J=12.4 Hz, CH_{2Bn-6}), 4.82 (d, 1H, J_{4,5}=4.0 Hz, H-5_{A}), 4.75 (brs, 2H, CH_{2Nap}), 4.44-4.38 (mₚₒ, 3H, H-2_{B}, CH_{2Bn}), 4.26 (dd, 1H, J_{3,4}=3.2 Hz, J_{2,3}=10.6 Hz, H-3_{B}), 4.22-4.09 (mₚₒ, 3H, H-2_{A}, H-4_{A}, H-5_{B}), 3.95 (d, 1H, H-4_{B}), 3.89 (dd, 1H, J_{2,3}=2.8 Hz, J_{3,4}=2.8 Hz, H-3_{A}), 3.44 (brd, 1H, J_{4,OH}=2.4 Hz, OH), 1.22 (d, 3H, J_{5,6}=6.4 Hz, H-6_{B}). ¹³C NMR (CDCl₃) δ 168.9 (C-6_{A}), 162.0, 161.9 (CO_{NHTCA}), 137.8, 137.0, 134.7, 134.5, 133.1 (2C) (C_{q,Ar}), 129.0, 128.8 (2C), 128.7, 128.6, 128.5, 128.4 (2C), 128.3, 128.2, 128.1, 127.9, 127.7, 127.1, 126.2, 126.1, 126.0, 125.3 (10C, C_{Ar}), 97.9 (C-1_{A}, ¹J_{C,H}=167 Hz), 92.4, 92.1 (2C, CCl₃), 91.1 (C-1_{B}, ¹J_{C,H}=175 Hz), 75.2 (C-3_{B}), 73.0 (C-3_{A}), 72.4 (C-5_{A}), 72.0 (C-4_{A}), 72.0 (CH_{2Nap}), 71.7 (CH_{2Bn}), 67.6 (CH_{2Bn-6}), 65.3 (C-5_{B}), 65.2 (C-4_{B}), 53.3 (C-2_{A}), 51.0 (C-2_{B}), 17.3 (C-6_{B}).

(Benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-α/β-D-galactopyranosyl N-phenyltrifluoroacetimidate (9a) and 2-trichloromethyl [(benzyl 3-O-benzyl-2-deoxy-4-O-(2-naphthylmethyl)-2-trichloroacetamido-α-L-altropyranosyluronate)-(1→3)-4-azido-1,2,4,6-tetradeoxy-α-D-galactopyrano]-[2,1]-oxazoline (10a). PTFACl (721 μL, 4.55 mmol, 1.3 equiv.) and Cs₂CO₃ (1.25 g, 3.85 mmol, 1.1 equiv.) were added to a solution of hemiacetal 9a (3.4 g, 3.50 mmol, 1.0 equiv.) in acetone (70 mL). After stirring for 2 h at rt, the suspension was filtered over a pad of Celite and solids were washed with acetone (30 mL) twice. The filtrate was concentrated under reduced pressure and the residue was purified by flash chromatography with cHex/EtOAc (90:1→80:20) to give a mix of the expected PTFA donor 9b and oxazoline 10b (3.6 g, 3.15 mmol, 91% calcd. wrt PTFA donor) as a white solid. Donor 9b had HRMS (ESI⁺): m/z [M+NH₄]⁺ calcd for C₄₉H₄₇Cl₆F₃N₇O₁₀, 1160.1462; found 1160.1445. Oxazoline 10b had HRMS (ESI⁺): m/z [M+NH₄]⁺ calcd for C₄₁H₃₈Cl₆N₅O₉, 954.0795; found 954.0776.

Oligomerization 2_{A}NTCA, 2_{B}NTCA (4_{A}-Nap)

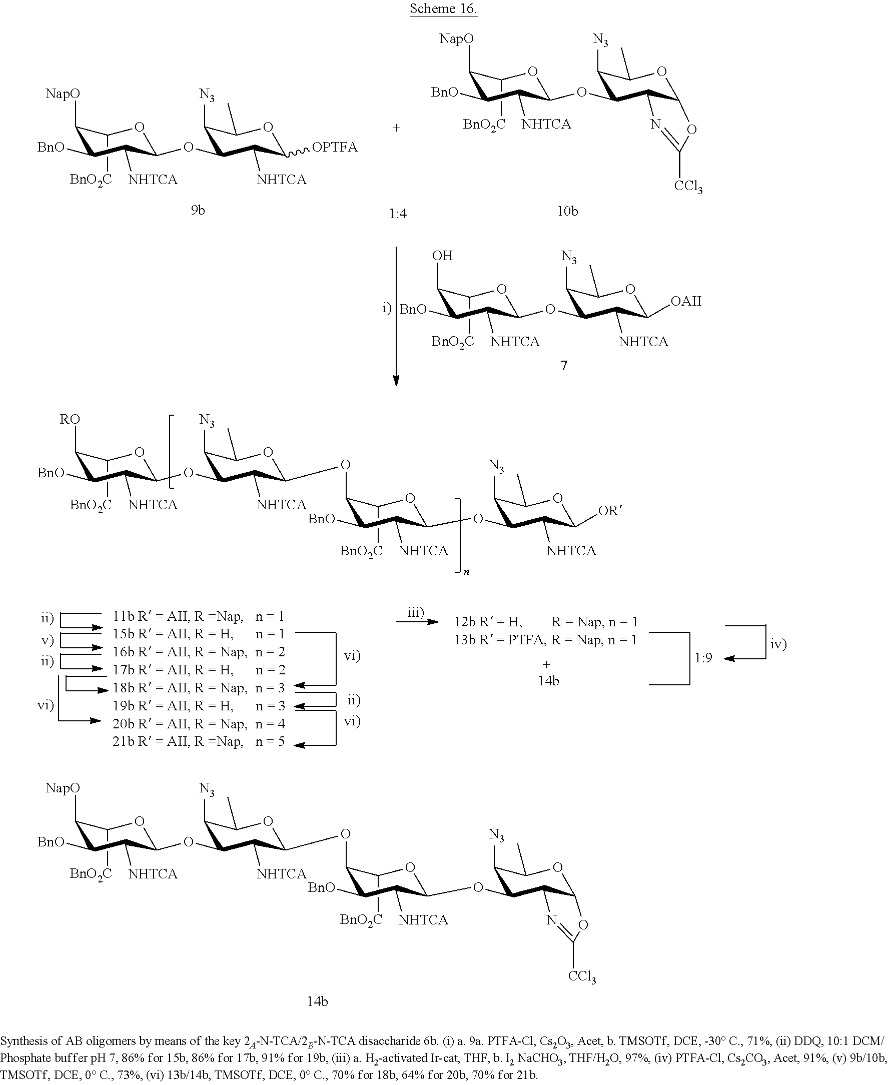

Allyl (benzyl 3-O-benzyl-2-deoxy-4-O-(2-naphthylmethyl)-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (11b). PTFACl (1.52 mL, 9.63 mmol, 1.3 equiv.) and Cs₂CO₃ (2.65 g, 8.15 mmol, 1.1 equiv.) were added to hemiacetal 9a (7.2 g, 7.41 mmol, 1.0 equiv.) in acetone (150 mL). After stirring for 2 h at rt, the reaction mixture was filtered through a pad of Celite and washed with DCM (50 mL) twice. The filtrate was concentrated under reduced pressure and dried under high vacuum to give the crude donors 9b/10b (8.4 g, 7.41 mmol, quant.), as a mix of PTFA donor 9b and oxazoline 10b. The crude material was used as such in the next step. Donors 9b/10b had R_{f} 0.95 (Tol/EtOAc 4:1).

A mix of the crude donors 9b/10b (8.4 g, 7.41 mmol, 1.06 equiv. theo.) and disaccharide acceptor 7 (6.0 g, 69.7 mmol, 1.0 equiv.) were co-evaporated with anhyd. toluene (50 mL) and then dried under vacuum. Freshly activated MS 4 Å (10 g) was added to the starting materials in anhyd. DCM (150 mL) and the suspension was stirred for 1 h under an Ar atmosphere at rt. After cooling to −15° C., TMSOTf (107 μL, 593 μmol, 0.08 equiv.) was added slowly and stirring went on for 45 min during which the bath temperature kept at −15° C. A TLC analysis (Tol/EtOAc 4:1) showed the absence of donors and the presence of a new spot in addition to a slight amount of less polar side products. At completion, Et₃N (120 μL) was added. The suspension was filtered through a fitted funnel and washed with DCM (50 mL) twice. Volatiles were evaporated and the residue was purified by flash chromatography (Tol/ACN 92:8→90:10) to give tetrasaccharide 11b as a white solid (9.1 g, 49.8 mmol, 71%). The coupling product 11b had R_{f} 0.45 (Tol/EtOAc 4:1). ¹H NMR (CDCl₃) δ 7.85-7.75 (m, 3H, H_{Ar}), 7.69 (brs, 1H, H_{Ar}), 7.43-7.20 (m, 26H, H_{Ar}), 6.97 (d, 1H, J_{2,NH}=6.8 Hz, NH_{B}), 6.77 (d, 1H, J_{2,NH}=7.2 Hz, NH_{B}), 6.57 (d, 1H, J_{2,NH}=7.6 Hz, NH_{A}), 6.49 (d, 1H, J_{2,NH}=7.2 Hz, NH_{B}), 5.90-5.80 (m, 1H, CH_{All}), 5.31-5.14 (m, 7H, H-1_{A}, CH_{2Bn-6}, CH_{2All}), 5.05 (d, 1H, J_{1,2}=5.2 Hz, H-1_{A}), 4.85 (dₚₒ, 1H, J_{1,2}=8.8 Hz, H-1_{B}), 4.83 (d, 1H, J_{4,5}=5.2 Hz, H-5_{A}), 4.80 (d, 1H, J_{1,2}=8.4 Hz, H-1_{B}), 4.74-4.66 (m, 5H, H-3_{B}, H-3_{B}, H-5_{A}, CH_{2Bn}), 4.59 (d, 1H, J=12.0 Hz, CH_{2Bn}), 4.48 (brd, 3H, CH_{2Bn}, CH_{2Nap}), 4.34-4.24 (m, 3H, H-2_{A}, H-4_{A}, CH_{2All}), 4.08-4.01 (m, 2H, H-4_{A}, CH_{2All}), 3.93-3.88 (m, 3H, H-2_{A}, H-3_{A}, H-4_{B}), 3.80-3.77 (m, 2H, H-3_{A}, H-4_{B}), 3.62-3.54 (m, 2H, H-2_{B}, H-5_{B}), 1.31 (d, 3H, J_{5,6}=6.0 Hz, H-6_{B}), 1.20 (d, 3H, J_{5,6}=6.0 Hz, H-6_{B}). ¹³C NMR (CDCl₃) δ 168.8, 168.2 (C-6_{A}), 162.0, 161.9, 161.6, 161.5 (CO_{NHTCA}), 137.4, 137.0, 135.1, 135.0, 133.4, 133.1 (2C) (C_{q,Ar}), 133.5 (CH_{All}), 129.1, 129.0, 128.7 (2C), 128.6, 128.5, 128.4, 128.3 (2C), 128.2, 128.0, 127.9, 127.7, 126.9, 126.2, 125.8, 125.2 (C_{Ar}), 99.2 (C-1_{A}, ¹J_{C,H}=170 Hz), 98.8 (C-1_{B}, ¹J_{C,H}=166 Hz), 98.1 (C-1_{A}, ¹J_{C,H}=170 Hz), 97.7 (C-1_{B}, ¹J_{C,H}=166 Hz), 92.4, 92.3, 92.1 (4C, CCl₃), 75.1 (C-3_{B}), 74.7 (C-3_{B}), 73.4 (C-3_{A}), 73.0 (C-3_{A}), 72.5 (CH_{2Nap}), 72.0 (2C, C-4_{A}), 71.9 (2C, CH_{2Bn}), 71.5 (2C, C-5_{A}), 70.0 (CH_{2All}), 69.3 (C-5_{B}), 68.8 (C-5_{B}), 67.6 (CH_{2Bn-6}), 67.4 (CH_{2Bn-6}), 65.5 (C-4_{B}), 65.1 (C-4_{B}), 55.6 (C-2_{B}), 55.5 (C-2_{B}), 54.1 (C-2_{A}), 52.7 (C-2_{A}), 17.3 (C-6_{B}). 17.2 (C-6_{B}). HRMS (ESI⁺): m/z [M+NH₄]⁺ calcd for C₇₄H₇₆Cl₁₂N₁₁O₁₉ 1842.1576; found 1842.1593.

Allyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (15b). Tetrasaccharide 11b (4.0 g, 2.19 mmol, 1.0 equiv.) was dissolved in DCM (40 mL) and phosphate buffer pH 7 (4.0 mL) was added. The biphasic mixture was cooled to 0° C. and DDQ (996 mg, 4.38 mmol, 2.0 equiv.) was added. The reaction was stirred for 6 h, keeping the temperature between 0-10° C. At completion, 10% aq. NaHCO₃ (100 mL) was added and the biphasic mixture was diluted with DCM (500 mL). The DCM layer was separated, washed with brine, dried over Na₂SO₄, filtered, and concentrated under reduced pressure. The residue was purified by flash chromatography (Tol/EtOAc 75:25→70:30) to give alcohol 15b (3.2 g, 1.90 mmol, 86%) as a white solid. The tetrasaccharide acceptor 15b had R_{f} 0.45 (Tol/EtOAc 7:3). HRMS (ESI⁺): m/z [M+NH₄]⁺ calcd for C₆₃H₆₈Cl₁₂N₁₁O₁₉ 1702.0950; found 1702.0950.

(Benzyl 3-O-benzyl-2-deoxy-4-O-(2-naphthylmethyl)-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-4-azido-2-trichloroacetamido-2,4,6-trideoxy-α/β-D-galactopyranose (12b). [Ir(COD)(PMePh₂)₂]PF₆ (185 mg, 219 μmol, 0.05 equiv.) in anhyd. THF (9.0 mL) was degassed and stirred for 30 min under an H₂ atmosphere. The resulting deep yellow solution was degassed repeatedly with Ar and poured into a solution of allyl glycoside 11b (8.0 g, 4.38 mmol, 1.0 equiv.) in anhyd. THF (80 mL). After stirring for 2 h at rt, a TCL follow up (Tol/EtOAc 4:1, 2 runs) revealed that the starting 11b (R_{f} 0.15) had been converted to a closely migrating product (R_{f} 0.2). NIS (1.18 g, 5.26 mmol, 1.2 equiv.) and H₂O (18 mL) were added and after stirring for another 1 h at rt, 10% aq. Na₂SO₃ was added. The reaction mixture was concentrated and the aq. phase was extracted with DCM (90 mL) thrice. The combined organic layers were washed with brine, dried over anhyd. Na₂SO₄, and concentrated under reduced pressure. The residue was purified by flash chromatography (cHex/EtOAc 80:20→75:25) to give the expected hemiacetal 12b (7.6 g, 7.51 mmol, 97%) as a white solid. The α/β hemiacetal 12b had R_{f} 0.1, 0.15 (Tol/EtOAc 4:1). HRMS (ESI⁺): m/z [M+NH₄]⁺ calcd for C₇₁H₇₂Cl₁₂N₁₁O₁₉ 1802.1263; found 1802.1242.

(Benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-trichloroacetamido-α-L-altropyranosyluronate)-(1→3)-4-azido-2-trichloroacetamido-2,4,6-trideoxy-α/β-D-galactopyranosyl (N-phenyl)trifluoroacetimidate (13b) and 2-trichloromethyl [(benzyl 3-O-benzyl-2-deoxy-4-O-(2-naphthylmethyl)-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-1,2,4,6-tetradeoxy-α-D-galactopyrano]-[2,1]-oxazoline (14b). PTFACl (878 μL, 5.53 mmol, 1.3 equiv.) and Cs₂CO₃ (1.52 g, 4.68 mmol, 1.1 equiv.) were added to a solution of hemiacetal 12b (7.6 g, 4.26 μmol, 1.0 equiv.) in acetone (85 mL). After stirring for 2 h at rt, the suspension was filtered over a pad of Celite and solids were washed with acetone (20 mL) twice. The filtrate was concentrated under reduced pressure and the residue was purified by flash chromatography (cHex/EtOAc 85:15→80:20) to give the expected PTFA tetrasaccharide 13b in admixture with the corresponding oxazoline 14b (7.5 g, 7.51 mmol, 91%, calcd. wrt PTFA donor) as a white solid. Imidate 13b had HRMS (ESI⁺): m/z [M+NH₄]⁺ calcd for C₇₉H₇₆Cl₁₂N₁₂O₁₉ 1973.1559; found 1973.1556. Oxazoline 14b had HRMS (ESI⁺): m/z [M+NH₄]⁺ calcd for C₇₁H₇₀Cl₁₂N₁₁O₁₈, 1784.1157; found 1784.1094.

Allyl (benzyl 3-O-benzyl-2-deoxy-4-O-(2-napthylmethyl)-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (16b). [4+2]-glycosylation: A mix of donors 9b/10b (678 mg, 594 μmol, 1.0 equiv.) and acceptor 15b (1.0 g, 594 μmol, 1.0 equiv.) were co-evaporated with anhyd. toluene and then dried under vacuum for 1 h. Freshly activated 4 Å MS (1.0 g) was added to the starting materials in anhyd. DCE (15 mL) and the suspension was stirred for 45 min under an Ar atmosphere at rt. After cooling to 0° C., TMSOTf (8.6 μL, 48 μmol, 0.08 equiv.) was added slowly and stirring went on for 45 min during which the bath temperature was kept at 0° C. A TLC analysis (Tol/EtOAc 4:1) showed the absence of donors and the presence of a new spot. At completion, Et₃N (10 μL) was added. The suspension was filtered through a fitted funnel and washed with DCM (20 mL) twice. Volatiles were evaporated and the residue was purified by flash chromatography (Tol/ACN 92:8→86:14) to give hexasaccharide 16b as a white solid (1.15 g, 436 μmol, 73%). The coupling product 16b had R_{f} 0.2 (Tol/EtOAc 4:1) HRMS (ESI⁺): m/z [M+NH₄]⁺ calcd for C₁₀₄H₁₀₅Cl₁₈N₁₆O₂₈ 2665.1580; found 2665.1580.

Allyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (17b). DDQ (155 mg, 683 μmol, 3.0 equiv.) was added to hexasaccharide 16b (600 mg, 228 μmol, 1.0 equiv.) in DCM (20 mL) and phosphate buffer pH 7 (2.0 mL). The biphasic mixture was cooled to 0° C. and stirred for 6 h while keeping the temperature between 0-10° C. A TLC analysis (Tol/EtOAc 3:1) showed the absence of the fully protected 16b (R_{f} 0.6) and the presence of a more polar spot (R_{f} 0.4). 10% Aq. NaHCO₃ (10 mL) was added followed by DCM (20 mL). The DCM layer was separated, washed with water and brine, dried over Na₂SO₄, filtered, and concentrated under reduced pressure. The residue was purified by flash chromatography (Tol/EtOAc 80:20→70:30) to give alcohol 17b (490 mg, 196 μmol, 86%) as a white solid. The hexasaccharide acceptor 17b had R_{f} 0.2 (Tol/EtOAc 4:1). HRMS (ESI⁺): m/z [M+NH₄]⁺ calcd for C₉₃H₉₇Cl₁₈N₁₆O₂₈ 2525.0942; found 2525.0949.

Allyl (benzyl 3-O-benzyl-2-deoxy-4-O-(2-napthylmethyl)-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (18b). [4+4]-glycosylation: Donors 13b/14b (1.21 g, 621 μmol, 1.0 equiv.) and acceptor 15b (1.04 g, 621 μmol, 1.0 equiv.) were co-evaporated with anhyd. toluene and then dried under vacuum for 1 h. Freshly activated 4 Å MS (2.0 g) was added to the starting materials in anhyd. DCE (15 mL) and the suspension was stirred for 45 min under an Ar atmosphere at rt. After cooling to 0° C., TMSOTf (10 μL, 565 μmol, 0.09 equiv.) was added slowly and stirring went on for 45 min during which the bath temperature was kept at 0° C. A TLC analysis (Tol/EtOAc 4:1) showed the absence of donors 13b/14b and the presence of a new spot. At completion, Et₃N (15 μL) was added. The suspension was filtered through a fitted funnel and washed with DCM (20 mL) twice. Volatiles were evaporated and the residue was purified by flash chromatography (Tol/ACN 88:12→84:16) to give octasaccharide 18b as a white solid (1.5 g, 434 μmol, 70%). The coupling product 18b had R_{f} 0.55 (Tol/EtOAc 7:3). HRMS (ESI⁺): m/z [M+2NH₄]²⁺ calcd for C₁₃₄H₁₃₈Cl₂₄N₂₂O₃₇ 1749.0997; found 1749.0984.

Allyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (19b). DDQ (223 mg, 983 μmol, 3.0 equiv.) was added to octasaccharide 18b (600 mg, 328 μmol, 1.0 equiv.) in DCM (30 mL) and phosphate buffer pH 7 (3.0 mL). The biphasic mixture was cooled to 0° C. and stirred for 6 h while keeping the temperature between 0-10° C. A TLC analysis (Tol/EtOAc 7:3) showed the absence of the fully protected 18b (R_{f} 0.6) and the presence of a more polar spot. 10% Aq. NaHCO₃ (30 mL) was added followed by DCM (30 mL). The DCM layer was separated, washed with water and brine, dried over Na₂SO₄, filtered, and concentrated under reduced pressure. The residue was purified by flash chromatography (Tol/EtOAc 80:20→70:30) to give alcohol 19b (990 mg, 299 μmol, 91%) as a white solid. The octasaccharide acceptor 19b had R_{f} 0.35 (Tol/EtOAc 7:3). HRMS (ESI⁺): m/z [M+2NH₄]²⁺ calcd for C₁₂₃H₁₂₉Cl₂₄N₂₂O₃₇ 1679.0681; found 1679.0663.

Allyl (benzyl 3-O-benzyl-2-deoxy-4-O-(2-napthylmethyl)-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (20b). [4+6]-glycosylation: Tetrasaccharide donors 13b/14b (235 mg, 120 μmol, 1.0 equiv.) and acceptor 17b (300 mg, 120 μmol, 1.0 equiv.) were co-evaporated with anhyd. toluene and then dried under vacuum. Freshly activated 4 Å MS (500 mg) was added to the starting materials in anhyd. DCE (10 mL) and the suspension was stirred for 45 min under an Ar atmosphere at rt. After cooling to 0° C., TMSOTf (2 μL, 11 μmol, 0.09 equiv.) was added slowly and stirring went on for 45 min during which the bath temperature kept at 0° C. A TLC analysis (Tol/EtOAc 4:1) showed the absence of donor 13b/14b and the presence of a new spot. At completion, Et₃N (2 μL) was added. The suspension was filtered through a fitted funnel and washed with DCM (20 mL) twice. Volatiles were evaporated and the residue was purified by flash chromatography (Tol/ACN 84:16→82:18) to give decasaccharide 20b as a white solid (330 mg, 77.4 μmol, 64%). The coupling product 20b had R_{f} 0.5 (Tol/EtOAc 7:3). HRMS (ESI⁺): m/z [M+2NH₄]²⁺ calcd for C₁₆₄H₁₇₅Cl₃₀N₂₇O₄₆ 2157.6027; found 2157.6028.

Allyl (benzyl 3-O-benzyl-2-deoxy-4-O-(2-napthylmethyl)-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→-3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (21b). [4+8]-glycosylation: The tetrasaccharide donors 13b/14b (490 mg, 251 μmol, 1.0 equiv.) and the octasaccharide acceptor 17b (830 mg, 251 μmol, 1.0 equiv.) were co-evaporated with anhyd. toluene and then dried under vacuum. Freshly activated 4 Å MS (1.0 g) was added to the starting materials in anhyd. DCE (15 mL) and the suspension was stirred for 1 h under an Ar atmosphere at rt. After cooling to 0° C., TMSOTf (4.1 μL, 23 μmol, 0.09 equiv.) was added slowly and stirring went on for 45 min during which the bath temperature kept at 0° C. A TLC analysis (Tol/EtOAc 7:3) showed the absence of donors 13b/14b and the presence of a new spot. At completion, Et₃N (5 μL) was added. The suspension was filtered through a fitted funnel and washed with DCM (15 mL) twice. Volatiles were evaporated and the residue was purified by flash chromatography (Tol/ACN 82:18→78:22) to give dodecasaccharide 21b as a white solid (900 mg, 177 μmol, 70%). The coupling product 21b had R_{f} 0.45 (Tol/ACN 4:1). HRMS (ESI⁺): m/z [M+2NH₄]²⁺ calcd for C₁₉₄H₁₉₆Cl₃₆N₃₂O₅₅ 2566.1056; found 2566.1059.

Full Deprotection from the Fully Protected Precursors Featuring a 4_{A}-Nap

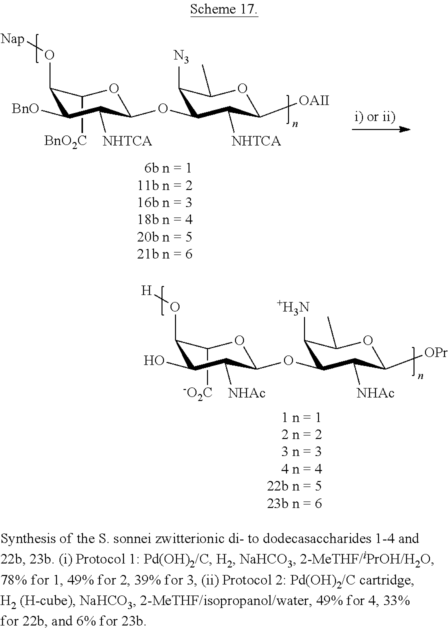

Propyl (2-acetamido-2-deoxy-α-L-altropyranosyluronic acid)-(1→3)-2-acetamido-4-amino-2,4,6-trideoxy-β-D-galactopyranoside (1). Disaccharide 6b (140 mg, 182 μmol) was subjected to deprotection (protocol 1). The known propyl glycoside 1 was obtained as a white lyophilized powder (66 mg, 130 μmol, 78%). The target propyl glycoside 1 had RP-HPLC (215 nm): Rₜ=12.3 min (conditions A), Rₜ=13.9 min (conditions B), Rₜ=12.2 min (conditions C). Analytical data were as above.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (2). Tetrasaccharide 11b (110 mg, 65 μmol) was subjected to hydrogenation (protocol 1). The desired propyl glycoside was obtained as a white lyophilized powder (28 mg, 32 μmol, 49%). Tetrasaccharide 2 had RP-HPLC (215 nm): Rₜ=12.3 min (conditions A′), Rₜ=13.6 min (conditions B). Analytical data were as above.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (3). The fully protected hexasaccharide 16b (100 mg, 38 μmol) was subjected to hydrogenation (protocol 1). The desired propyl glycoside 3 was obtained as a white lyophilized solid (19 mg, 15 μmol, 39%). The free hexasaccharide 3 had RP-HPLC (215 nm): Rₜ=11.3 min (conditions A′), Rₜ=13.4 min (conditions B). Analytical data were as above.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (4). The fully protected octasaccharide 18b (20 mg, 6 μmol) was subjected to deprotection (protocol 2). A solution of the starting material in iPrOH/MeTHF/H₂O (10:1:1) was passed through a 20% Pd(OH)₂—C cartridge at a flow of 0.8 mL/min in the full H₂ mode. After the first run was over, 0.12 mM aq. NaHCO₃ (50 μL, 6 μmol, 1 equiv.) was added. And the same was repeated over the next five runs to reach a total of 5 equiv. of NaHCO₃. The product was obtained as a white solid (4.9 mg, 2.9 μmol, 50%). The free octasaccharide 4 had RP-HPLC (215 nm): Rₜ=13.3 min (conditions A).

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (22b). The fully protected decasaccharide 20b (30 mg, 7.0 μmol) was subject to hydrogenation (protocol 2). The product was obtained as a white lyophilized foam (4.9 mg, 2.4 μmol, 33%). The free decasaccharide 22b had RP-HPLC (215 nm): Rₜ=13.5 min (conditions A). HRMS (ESI⁺): m/z [M+2H]²⁺ calcd for C₈₃H₁₃₅N₁₅O₄₆ 1038.9337; found 1038.9330.

Propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (23b). The fully protected dodecasaccharide 21b (50 mg, 10.7 μmol) was subjected to one step hydrogenation-mediated final deprotection (protocol 2). The free propyl glycoside was isolated as a white lyophilized material (1.5 mg, 0.61 μmol, 6%). The free dodecasaccharide 23b had RP-HPLC (215 nm): Rₜ=14.1 min (conditions A). HRMS (ESI⁺): m/z [M+3H]³⁺ calcd for C₉₉H₁₆₁N₁₈O₅₅ 827.6790; found 827.6793.

### Example 7: Strategy 2_{A}-NTCA,2_{B}-NTCA, Featuring a 4_{A}-Me Endchain Disaccharide

The Chain Terminator 4_{A}-Me AB Disaccharide Building Block

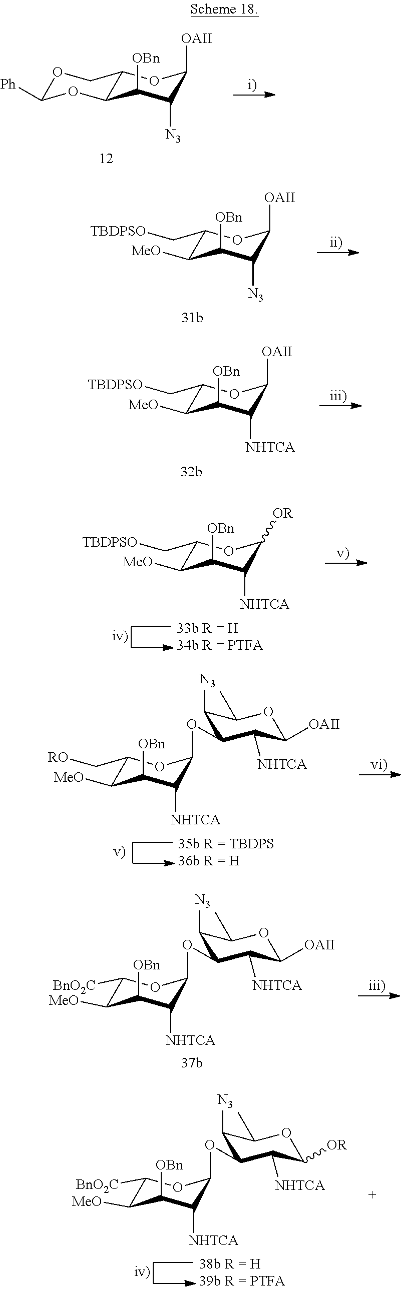

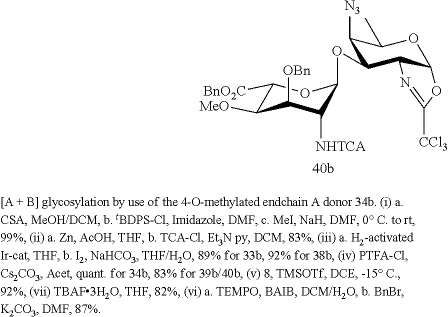

Allyl 2-azido-3-O-benzyl-6-O-tert-butyldiphenylsilyl-2-deoxy-4-O-methyl-α-L-altropyranoside (31b). CSA (1.3 g, 5.90 mmol, 0.5 equiv.) was added to the benzylidene acetal 12 (5.0 g, 11.8 mmol, 1.0 equiv.) in MeOH/DCM (4:1, 50 mL). After stirring at rt for 2 h, a TLC follow up (Tol/EtOAc 4:1) indicated reaction completion as shown by the absence of the starting material (R_{f} 0.65) and the presence of a polar spot. 10% Aq. NaHCO₃ (100 mL) was added followed by EtOAc (100 mL). The organic phase was separated and washed with brine (100 mL). The organic phase was dried over Na₂SO₄ and concentrated under reduced pressure. The crude product was dried under high vacuum. tert-Butyldiphenylchlorosilane (3.37 mL, 12.9 mmol, 1.1 equiv.) and imidazole (1.6 g, 23.6 mmol, 2.0 equiv.) were added to the crude diol intermediate in anhyd. DMF (60 mL) at 0° C. The reaction mixture was allowed to reach rt slowly and stirred overnight at this temperature. Methanol (5.0 mL) was added and after 30 min, volatiles were evaporated under reduced pressure. The residue was dissolved in EtOAc and the organic layer was washed with 90% aq. brine, separated, dried over Na₂SO₄, and concentrated to provide the crude 6-O-silylated intermediate. Mel (1.47 g, 23.6 mmol, 2.0 equiv.) was added to the crude intermediate in DMF (230 mL). The solution was cooled to 0° C. and NaH (60% in mineral oil, 567 mg, 23.6 mmol, 2.0 equiv.) was added portionwise. After stirring vigorously for 2 h while the bath temperature slowly reached rt, a TLC follow up indicated reaction completion. The reaction mixture was diluted with DCM (1 L) and 5% aq. NH₄Cl (100 mL) was added. The organic layer was washed with water and brine, dried over Na₂SO₄ and concentrated. The crude product was purified by flash chromatography (cHex/EtOAc 92:8→88:12) to give the fully protected 31b (6.9 g, 11.7 mmol, 99%) as a light yellow oil. Allyl glycoside 31b had R_{f} 0.65 (Tol/EtOAc 10:1). ¹H NMR (CDCl₃) δ 7.71-7.68 (m, 4H, H_{Ar}), 7.47-7.31 (m, 11H, H_{Ar}), 5.95-5.90 (m, 1H, CH_{All}), 5.34-5.29 (m, 1H, CH_{2All}), 5.21-5.18 (m, 1H, CH_{2All}), 4.76-4.67 (m, 3H, H-1, CH_{2Bn}), 4.30-4.25 (m, 1H, CH_{2All}), 4.12 (q, 1H, J_{5,6b}=4.8 Hz, H-5), 4.07-4.02 (m, 1H, CH_{2All}), 3.90 (dd, 1H, J_{1,2}=4.8 Hz, J_{2,3}=8.0 Hz, H-2), 3.81 (brd, 2H, J_{5,6}=4.4 Hz, H-6a, H-6b), 3.77 (dd, 1H, J_{3,4}=3.6 Hz, H-3), 3.66 (dd, 1H, J_{4,5}=5.2 Hz, H-4), 3.39 (s, 3H, OCH₃), 1.09 (s, 9H, CH_{3,TBDPS}). ¹³C NMR (CDCl₃) δ 137.8, 133.2, 133.0 (C_{q,Ar}), 133.8 (CH_{All}), 135.7, 135.5, 129.8, 128.4, 127.8, 127.7 (2C), 127.5 (C_{Ar}), 117.3 (CH_{2All}), 98.7 (C-1_{A}, ¹J_{C,H}=169 Hz), 75.8 (C-3), 74.9 (C-4), 72.5 (C-5), 72.3 (CH_{2Bn}), 68.7 (CH_{2All}), 63.9 (C-6), 61.7 (C-2), 58.2 (OCH₃), 26.8 (CH_{3,TBDPS}), 19.2 (C_{TBDPS}). HRMS (ESI⁺): m/z [M+NH₄]⁺ calcd for C₃₃H₄₅N₄O₅Si, 605.3154; found 605.3153.

Allyl 3-O-benzyl-6-O-tert-butyldiphenylsilyl-2-deoxy-4-O-methyl-2-trichloroacetamido-α-L-altropyranoside (32b). Zn dust (7.6 g, 117 mmol, 10.0 equiv.) and AcOH (6.7 mL, 117 mmol, 10.0 equiv.) were added to azide 31b (6.9 g, 11.7 mmol, 1.0 equiv.) in anhyd. THF (120 mL). After stirring for 1 h at rt, a TLC analysis (Tol/EtOAc 10:1) showed the absence of azide 31b (R_{f} 0.8) and the presence of a more polar spot. The suspension was filtered over a pad of Celite and washed with THF. The filtrate was concentrated and then diluted with DCM. The DCM layer was washed with satd aq. NaHCO₃, water, and brine, dried over Na₂SO₄, concentrated under reduced pressure, and dried under high vacuum. Trichloroacetyl chloride (1.97 mL, 17.6 mmol, 1.5 equiv.) and triethylamine (3.25 mL, 23.4 mmol, 2.0 equiv.) were added to a solution of the crude amine in DCM (120 mL). After stirring at rt for 1 h, a TLC follow up (Tol/EtOAc 10:1) indicated reaction completion. Methanol (2 mL) was added and the reaction mixture was stirred for another 10 min. Volatiles were evaporated and dried under high vacuum. The crude was taken in DCM (300 mL) and the DCM layer was washed with water and brine, dried over Na₂SO₄, filtered, and concentrated under reduced pressure. The residue was purified by flash chromatography (cHex/EtOAc 94:6→90:10) to give the fully protected 32b (6.95 g, 118 mmol, 83%) as a yellowish dense oil. Allyl glycoside 32b had R_{f} 0.7 (Tol/EtOAc 10:1). ¹H NMR (CDCl₃) δ 7.75-7.71 (m, 4H, H_{Ar}), 7.47-7.29 (m, 11H, H_{Ar}), 6.84 (d, 1H, J_{2,NH}=8.4 Hz, NH), 5.95-5.86 (m, 1H, CH_{All}), 5.34-5.29 (m, 1H, CH_{2All}), 5.20-5.17 (m, 1H, CH_{2All}), 4.88 (dₚₒ, 1H, J=12.0 Hz, CH_{2Bn}), 4.87 (d, 1H, J_{1,2}=4.8 Hz, H-1), 4.70 (d, 1H, CH_{2Bn}), 4.48 (ddd, 1H, J_{2,3}=3.6 Hz, H-2), 4.27 (m, 2H, H-5, CH_{2All}), 4.06-3.98 (mₚₒ, 2H, H-6a, CH_{2All}), 3.90 (dd, 1H, J_{5,6b}=2.4 Hz, J_{6a,6b}=11.2 Hz, H-6b), 3.67 (dd, 1H, J_{4,5}=9.2 Hz, H-4), 3.27 (s, 3H, OCH₃), 1.09 (s, 9H, CH_{3,TBDPS}). ¹³C NMR (CDCl₃) δ 161.2 (CO_{NHTCA}), 137.9, 133.5, 133.3 (C_{q,Ar}), 133.5 (CH_{All}), 135.7, 133.5, 129.6, 128.2, 128.1, 127.6 (15C, C_{Ar}), 117.3 (CH_{2All}), 97.3 (C-1_{A}, ¹J_{C,H}=169 Hz), 73.0 (C-4), 71.5 (CH_{2Bn}), 71.2 (C-3), 68.5 (C-5), 68.3 (CH_{2All}), 62.8 (C-6), 56.4 (OCH₃), 50.8 (C-2), 26.8 (CH_{3,TBDPS}), 19.4 (C_{TBDPS}). HRMS (ESI⁺): m/z [M+NH₄]⁺ calcd for C₃₅H₄₆Cl₃N₂O₆Si, 723.2185; found 723.2184.

3-O-Benzyl-6-O-tert-butyldiphenylsilyl-2-deoxy-4-O-methyl-2-trichloroacetamido-α-L-altropyranose (33b). [Ir(COD)(PMePh₂)₂]⁺PF₆⁻ (248 mg, 294 μmol, 0.03 equiv.) was dissolved in anhyd. THF (10 mL) and stirred for 30 min under an H₂ atmosphere. The resulting yellow solution was degassed several times and poured into a solution of allyl glycoside 32b (6.9 g, 9.78 mmol, 1.0 equiv.) in anhyd. THF (90 mL) under Ar. After stirring for 2 h at rt, a solution of NaHCO₃ (2.4 g, 29.3 mmol, 3.0 equiv) in H₂O (10 mL) was added, followed by a solution of iodine (4.96 g, 19.5 mmol, 2.0 equiv) in THF (10 mL). After stirring for 1 h at rt, the reaction was quenched with 10% aq. Na₂SO₃. The reaction mixture was concentrated and the aq. phase was extracted with DCM (3×100 mL). The organic phases were pooled, washed with brine, dried over anhyd. Na₂SO₄ and concentrated. Purification by flash chromatography (cHex/EtOAc 90:10→88:12) gave hemiacetal 33b (α/β-4:1, 5.8 g, 8.71 mmol, 89%) as a white foam. The α/β-anomer 33b had R_{f} 0.55, 0.5 (Tol/EtOAc 4:1); ¹H NMR (CDCl₃) δ 7.75-7.69 (m, 5.2H, H_{Ar}), 7.47-7.26 (m, 14.4H, H_{Ar}), 7.21-7.18 (m, 0.56H, H_{Ar}), 7.09 (d, 1H, J_{2,NH}=6.4 Hz, NH_{α}), 6.84 (d, 0.24H, J_{2,NH}=7.6 Hz, NH_{β}), 5.61 (d, 0.24H, J_{1,OH}=11.2 Hz, OH_{β}), 5.37 (dd, 1H, H-1_{α}), 5.10 (ddₚₒ, 0.24H, H1_{β}), 4.90-4.75 (m, 2.54H, CH_{2Bn,α}, CH_{2Bn,β}), 4.39-4.36 (m, 0.51H, H-2_{β}, H-5β), 4.31-4.25 (m, 2H, H-2_{α}, H-5_{α}), 4.13-4.07 (m, 0.54H, H-6_{αβ}, H-4β), 3.98-3.88 (m, 3.3H, H-4_{α}, H-6ab_{α,β}, H-6b_{β}), 3.80 (dd, 0.28H, J_{2,3}=2.4 Hz, J_{3,4}=9.6 Hz, H-3β), 3.65 (dd, 1H, J_{2,3}=3.2 Hz, J_{3,4}=9.2 Hz, H-3_{α}), 3.32 (s, 0.76H, OCH_{3,β}), 3.29 (s, 3H, OCH_{3,α}), 3.06 (d, 1H, J_{1,OH}=4.0 Hz, OH_{α}), 1.09, 1.08 (2s, 11.6H, CH_{3,TBDPS}). ¹³C NMR (CDCl₃) δ 162.2, 161.4 (CO_{NHTCA}), 137.8, 136.5, 135.6, 135.5 (2C), 133.6, 133.3, 133.2, 133.1 (C_{q,Ar}), 135.7, 135.6, 135.5 (2C), 129.7 (2C), 129.0, 128.7, 128.5, 128.4, 128.3, 128.0, 127.9, 127.7, 127.6, 125.3 (C_{Ar}), 93.4 (C-1_{β}, ¹J_{C,H}=174 Hz), 91.3 (C-1_{A}, ¹J_{C,H}=169 Hz), 74.3 (CH_{2Bn,β}), 74.1 (C-4_{α}), 73.5 (C-3_{α}), 73.0 (CH_{2Bn,β}), 72.8 (C-3_{β}), 72.7 (C-5_{β}), 71.3 (C-5_{α}), 67.6 (C-4_{β}), 62.9 (C-6_{α}), 62.9 (C-6_{β}), 57.1 (OCH₃), 53.1 (C-2_{α}), 51.3 (C-2_{β}), 26.9, 26.8 (CH_{3, TBDPS}), 19.4 (C_{TBDPS}). HRMS (ESI⁺): m/z [M+NH₄]⁺ calcd for C₃₂H₄₂Cl₃N₂O₆Si, 683.1872; found 683.1876.

Allyl 3-O-benzyl-6-O-tert-butyldiphenylsilyl-2-deoxy-4-O-methyl-2-trichloroacetamido-α-L-altropyranosyl-(1→3)-4-azido-2-trichloroacetamido-2,4,6-trideoxy-β-D-galactopyranoside (35b). Hemiacetal 33b (5.8 g, 8.72 mmol, 1.0 equiv.) was dissolved in acetone (80 mL). PTFACl (1.79 mL, 11.3 mmol, 1.3 equiv.) was added followed by Cs₂CO₃ (3.4 mg, 10.4 mmol, 1.2 equiv). After stirring for 2 h at rt, the reaction mixture was filtered and the filtrate was concentrated under reduced pressure. The PTFA donor 34b had R_{f} 0.8 (Tol/EtOAc 4:1).

The crude PTFA donor 34b (7.29 g, 8.72 mmol, 1.0 equiv. theo.) and acceptor 8 (3.2 g, 8.72 mmol, 1.0 equiv) were co-evaporated with anhyd. toluene (40 mL) and dried under vacuum. The dried mass was dissolved in anhyd. DCE (120 mL), freshly activated 4 Å MS (5.0 g) was added and the suspension was stirred for 30 min at rt under an Ar atmosphere. The reaction mixture was cooled to −15° C. and TMSOTf (79 μL, 436 μmol, 0.05 equiv.) was added. After 40 min at −15° C., a TLC analysis (Tol/EtOAc 4:1) showed the absence of donor and the presence of a new spot. The mixture was quenched with Et₃N, filtered and concentrated. Flash chromatography (cHex/EtOAc 90:10→88:12) yielded the coupling product 35b (8.2 g, 8.04 mmol, 92%) as a white foam. The desired disaccharide 35b had R_{f} 0.5 (Tol/EtOAc 4:1). ¹H NMR (CDCl₃) δ 7.73-7.70 (m, 4H, H_{Ar}), 7.52-7.28 (m, 11H, H_{Ar}), 6.73 (d, 1H, J_{2,NH}=7.2 Hz, NH_{B}), 6.67 (d, 1H, J_{2,NH}=8.0 Hz, NH_{A}), 5.90-5.81 (m, 1H, CH_{All}), 5.29-5.24 (m, 1H, CH_{2All}), 5.21-5.17 (m, 1H, CH_{2All}), 4.92 (d, 1H, J_{1,2}=8.4 Hz, H-1_{B}), 4.85 (dₚₒ, 1H, J_{1,2}=4.4 Hz, H-1_{A}), 4.84 (dₚₒ, 1H, J=12.4 Hz, CH_{2Bn}), 4.77 (d, 1H, CH_{2Bn}), 4.59 (dd, 1H J_{2,3}=10.8 Hz, J_{3,4}=4.0 Hz, H-3_{B}), 4.41-4.30 (m, 3H, H-2_{A}, H-5_{A}, CH_{2All}), 4.08-4.01 (m, 3H, H-4_{A}, H-6a_{A}, CH_{2All}), 3.95 (dd, J_{5,6b}=2.0 Hz, J_{6a,6b}=11.2 Hz, H-6b_{A}), 3.63 (dₚₒ, 1H, J_{4,5}=3.6 Hz, H-4_{B}), 3.60 (dqₚₒ, 1H, J_{4,5}=1.0 Hz, H-5_{B}), 3.53 (dd, 1H, J_{2,3}=9.2 Hz, J_{3,4}=3.6 Hz, H-3_{A}), 3.43-3.36 (m, 1H, H-2_{B}), 3.29 (s, 3H, OCH₃), 1.23 (d, 3H, 6.4 Hz, H-6_{B}), 1.09 (s, 9H, CH_{3,TBDPS}). ¹³C NMR (CDCl₃) δ 162.2, 161.1 (CO_{NHTCA}), 138.4, 133.4, 133.3 (C_{q,Ar}), 133.5 (CH_{All}), 135.7, 135.5, 129.8, 128.8, 128.2, 128.7 (C_{Ar}), 117.8 (CH_{2All}), 100.6 (C-1_{A}, ¹J_{C,H}=169 Hz), 97.3 (C-1_{B}, ¹J_{C,H}=163 Hz), 92.1, 92.0 (2C, CCl₃), 76.2 (C-3_{B}), 73.3 (C-3_{A}), 72.3 (CH_{2Bn}), 72.0 (C-4_{A}), 70.1 (CH_{2All}), 69.7 (C-5_{B}), 69.4 (C-5_{A}), 65.6 (C-4_{B}), 63.2 (C-6_{A}), 56.7 (OCH₃), 56.0 (C-2_{B}), 51.4 (C-2_{A}), 26.9 (CH_{3,TBDPS}), 19.4 (C_{TBDPS}), 17.2 (C-6_{B}). HRMS (ESI⁺): m/z [M+NH₄]⁺ calcd for C₄₃H₅₅Cl₆N₆O₉Si, 1037.1925; found 1037.1914.

Allyl 3-O-benzyl-2-deoxy-4-O-methyl-2-trichloroacetamido-α-L-altropyranosyl-(1→3)-4-azido-2-trichloroacetamido-2,4,6-trideoxy-β-D-galactopyranoside (36b). TBAF·3H₂O (2.91 g, 9.25 mmol, 1.15 equiv.) was added to disaccharide 35b (8.2 g, 8.0 mmol, 1.0 equiv.) in THF (80 mL) and the reaction mixture was stirred at rt for 2 h. A TLC analysis (EtOAc) showed the consumption of the protected disaccharide 35b (R_{f} 1.0) and the presence of a polar spot. Acetic acid (0.53 mL, 9.25 mmol, 1.15 equiv.) was added slowly and after stirring for 10 min, volatiles were evaporated. The residue was purified by flash chromatography (cHex/EtOAc 30:70→0:100) to give alcohol 36b (5.8 g, 7.42 mmol, 92%) as a white solid. Disaccharide 36b had R_{f} 0.35 (EtOAc). ¹H NMR (DMSO-d₆) δ 8.92 (d, 1H, J_{2,NH}=8.4 Hz, NH_{A}), 8.82 (d, 1H, J_{2,NH}=9.2 Hz, NH_{B}), 7.37-7.24 (m, 5H, H_{Ar}), 5.85-5.75 (m, 1H, CH_{All}), 5.25-5.19 (m, 1H, CH_{2All}), 5.11-5.08 (m, 1H, CH_{2All}), 5.00 (d, 1H, J_{1,2}=4.0 Hz, H-1_{A}), 4.82 (t, 1H, J_{6,OH}=5.6 Hz, OH), 4.61 (d, 1H, J=11.6 Hz, CH_{2Bn}), 4.55 (d, 1H, CH_{2Bn}), 4.48 (d, 1H, J_{1,2}=8.4 Hz, H-1_{B}), 4.22-4.14 (m, 3H, H-3_{B}, H-2_{A}, CH_{2All}), 4.08-4.02 (m, 2H, H-4_{B}, H-5_{A}), 3.98-3.93 (m, 1H, CH_{2All}), 3.86-3.79 (m, 2H, H-4_{A}, H-2_{B}), 3.69-3.60 (mₚₒ, 2H, H-5_{B}, H-6a_{A}), 3.58-3.54 (mₚₒ, 2H, H-3_{A}, H-6b_{A}), 3.24 (s, 3H, OCH₃), 1.23 (d, 3H, 6.4 Hz, H-6_{B}). ¹³C NMR (DMSO-d₆) δ 162.0, 161.8 (CO_{NHTCA}), 138.8 (C_{q,Ar}), 134.8 (CH_{All}), 128.4, 128.0, 127.7 (C_{Ar}), 116.9 (CH_{2All}), 100.8 (C-1_{A}, ¹J_{C,H}=172 Hz), 100.4 (C-1_{B}, ¹J_{C,H}=161 Hz), 93.6, 93.2 (2C, CCl₃), 76.3 (C-3_{B}), 74.5 (C-3_{A}), 74.0 (C-4_{A}), 72.7 (C-5_{A}), 71.4 (CH_{2Bn}), 69.5 (CH_{2All}), 69.3 (C-5_{B}), 65.4 (C-4_{B}), 61.7 (C-6_{A}), 57.6 (OCH₃), 53.4 (C-2_{B}), 52.7 (C-2_{A}), 17.7 (C-6_{B}). HRMS (ESI⁺): m/z [M+NH₄]⁺ calcd for C₂₇H₃₇Cl₆N₆O₉, 799.0748; found 799.0748.

Allyl (benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranoside (37b). TEMPO (234 mg, 1.49 mmol, 0.2 equiv) and BAIB (6.0 g, 18.7 mmol, 2.5 equiv) were added to a solution disaccharide 36b (5.85 g, 7.49 mmol, 1.0 equiv.) in biphasic DCM/H₂O (1:10, 150 mL). After stirring for 2 h at rt, the reaction was quenched with 10% aq. Na₂SO₃ and the biphasic mixture was diluted with DCM (150 mL). The aq. phase was separated and extracted twice with DCM (150 mL). The combined organic phases were washed with brine, dried by passing through a phase separator filter and concentrated under reduced pressure. To a solution of the crude material in anhyd. DMF (40 mL) were added benzyl bromide (2.66 mL, 22.4 mmol, 3.0 equiv) and K₂CO₃ (1.3 g, 9.73 mmol, 1.3 equiv.). After stirring for 1 h at rt, the reaction mixture was diluted with 10% aq. NH₄Cl (100 mL) and extracted with DCM (200 mL) twice. The organic phases were pooled, washed with brine, dried over anhyd Na₂SO₄, filtered and concentrated in vacuo. Flash chromatography using Tol/EtOAc (4:1) gave the benzyl ester 37b (5.8 g, 6.55 mmol, 87%) as a white solid. Uronate 37b had R_{f} 0.6 (Tol/EtOAc 4:1). ¹H NMR (CDCl₃) δ 7.44-7.28 (m, 10H, H_{Ar}), 6.78 (d, 1H, J_{2,NH}=7.2 Hz, NH_{B}), 6.73 (d, 1H, J_{2,NH}=7.6 Hz, NH_{A}), 5.88-5.80 (m, 1H, CH_{All}), 5.30-5.23 (mₚₒ, 3H, CH_{2Bn-6}, CH_{2All}), 5.22 (dₚₒ, 1H, J_{1,2}=6.0 Hz, H-1_{A}), 5.19-5.16 (m, 1H, CH_{All}), 4.82 (d, 1H, J_{1,2}=8.4 Hz, H-1_{B}), 4.78 (d, 1H, J_{4,5}=5.2 Hz, H-5_{A}), 4.65-4.53 (m, 3H, H-3_{B}, CH_{2Bn}), 4.34-4.29 (m, 1H, CH_{2All}), 4.14-4.08 (m, 1H, H-2_{A}), 4.06-4.01 (m, 1H, CH_{2All}), 3.97 (dd, 1H, J_{2,3}=8.4 Hz, J_{3,4}=2.8 Hz, H-3_{A}), 3.85-3.83 (m, 2H, H-4_{B}, H-4_{A}), 3.60-3.52 (m, 2H, H-2_{B}, H-5_{B}), 3.38 (s, 3H, OCH₃), 1.27 (d, 3H, J_{5,6}=6.0 Hz, H-6_{B}). ¹³C NMR (CDCl₃) δ 168.9 (CO_{CO2Bn}), 161.9, 161.7 (CO_{NHTCA}), 137.4, 135.0 (C_{q,Ar}), 133.5 (CH_{All}), 128.7 (3C), 128.4, 128.1, 128.0 (C_{Ar}), 117.9 (CH_{2All}), 98.7 (C-1_{A}, ¹J_{C,H}=170 Hz), 97.7 (C-1_{B}, ¹J_{C,H}=164 Hz), 92.3, 92.2 (2C, CCl₃), 75.8 (C-3_{B}), 75.1 (C-4_{A}), 73.2 (C-3_{A}), 72.1 (CH_{2Bn}), 71.4 (C-5_{A}), 70.1 (CH_{2All}), 69.3 (C-5_{B}), 67.5 (CH_{2Bn-6}), 65.2 (C-4_{B}), 58.2 (OCH₃), 55.5 (C-2_{B}), 53.3 (C-2_{A}), 17.3 (C-6_{B}). HRMS (ESI⁺): m/z [M+NH₄]⁺ calcd for C₃₄H₄₁Cl₆N₆O₁₀, 903.1010; found 903.1014.

(Benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranose (38b). [Ir(COD)(PMePh₂)₂]⁺PF₆⁻ (149 mg, 0.176 mmol, 0.03 equiv) was dissolved in anhyd THF (10 mL) and stirred for 30 min under an H₂ atmosphere. The resulting yellow solution was degassed several times and poured into a solution of the allyl glycoside 31b (5.2 mg, 5.87 mmol, 1.0 equiv.) in anhyd THF (80 mL). After stirring for 2 h at rt, a solution of iodine (2.9 g, 11.7 mmol, 2.0 equiv) and NaHCO₃ (1.48 g, 17.6 mmol, 3.0 equiv.) were added. After stirring for 2 h at rt, the reaction was quenched with 10% aq sodium sulphite. The reaction mixture was concentrated and the aq phase was extracted with DCM (3×150 mL). The organic phases were pooled, washed with brine, dried over anhyd. Na₂SO₄ and concentrated. Purification by flash chromatography (Tol/EtOAc 75:25→70:30) yielded hemiacetal 38b (α/β 3.5:1, 4.6 g, 5.44 mmol, 92%) as a white foam. The α/β-anomer had R_{f} 0.2, 0.5 (Tol/EtOAc 7:3). ¹H NMR (CDCl₃) δ 7.45-7.18 (m, 15H, HA, NH_{B-β}), 7.04 (d, 1H, J_{2,NH}=8.4 Hz, NH_{B-α}), 6.87 (d, 1H, J_{2,NH}=6.8 Hz, NH_{A-α}), 6.83 (d, 1H, J_{2,NH}=6.8 Hz, NH_{A-β}), 5.43 (d, 1H, J_{1,2}=6.8 Hz, H-1_{A-α}), 5.39 (d, 0.3H, J_{1,2}=6.8 Hz, H-1_{A-β}), 5.29 (tₚₒ, 1.1H, J_{1,2}=3.6 Hz, H-1_{B-α}), 5.6-5.21 (mₚₒ, 2.6H, CH_{2Bn-6}), 4.81 (d, 0.3H, J_{4,5}=3.6 Hz, H-5_{A-β}), 4.79 (d, 1H, J_{4,5}=3.2 Hz, H-5_{A-α}), 4.67 (t, 0.3H, J_{1,2}=8.8 Hz, H-1_{Bβ}), 4.61-4.37 (m, 2.7H, CH_{2Bn}), 4.43-4.37 (m, 1H, H-2_{B-α}), 4.26 (dd, 1H, J_{2,3}=8.4 Hz, J_{3,4}=3.2 Hz, H-3_{B-α}), 4.17 (dd, 1H, J_{2,3}=8.4 Hz, J_{3,4}=3.2 Hz, H-3_{B-β}), 4.13-4.03 (m, 2.7H, H-5_{B-α}, H-2_{A-α}, H-2_{A-β}), 3.96-3.86 (m, 4H, H-4_{B-α}, H-4_{A-α}, H-3_{A-α}, H-2_{A-β}, H-3_{A-β}, H-4_{B-β}), 3.82 (dd, 0.3H, J_{3,4}=2.8 Hz, J_{4,5}=9.6 Hz, H-4_{A-β}), 3.62-3.59 (m, 0.3H, H-5_{B-β}), 3.54 (s, 1H, OCH_{3-β}), 3.41 (s, 1H, OCH_{3-α}), 3.07 (d, 1H, J_{1,OH}=3.6 Hz, OH), 1.32 (d, 1H, J_{5,6}=6.4 Hz, H-6_{B-β}) 1.27 (d, 3H, J_{5,6}=6.4 Hz, H-6_{B-α}). ¹³C NMR (CDCl₃) δ 168.9, 168.7 (CO_{CO2Bn}), 162.2, 162.0, 161.8 (CO_{NHTCA}), 137.0, 136.8, 134.8 (C_{Ar}), 129.0, 128.9, 128.8 (2C), 128.7, 128.6, 128.5, 128.3, 128.2, 128.1 (C_{Ar}), 98.4 (C-1_{A}-β, ¹J_{C,H}=168 Hz), 97.5 (C-1_{A-α}, ¹J_{C,H}=169 Hz), 96.1 (C-1_{B-β}, ¹J_{C,H}=163 Hz), 96.1 (C-1_{B-α}, ¹J_{C,H}=176 Hz), 92.5, 92.2 (2C, CCl₃), 76.5 (C-3_{B-α}), 75.2 (C-3_{B-α}), 75.1 (C-4_{A-α}), 75.1 (C-4_{A-β}), 73.8 (C-3_{A-β}), 73.1 (C-3_{A-α}), 72.8 (C-5_{A-β}), 72.2 (C-5_{A-α}), 71.9 (CH_{2Bn-β}), 71.8 (CH_{2Bn-β}), 69.7 (C-5_{B-β}), 67.8 (CH₂Bn-6β), 67.7 (CH_{2Bn-6α}), 65.4 (C-5_{B-α}), 65.0 (C-4_{B-α}), 64.5 (C-4_{B-β}), 58.4 (OCH_{3-β}), 58.0 (OCH_{3-α}), 55.7 (C-2_{A-β}), 53.6 (C-2_{A-α}), 53.5 (C-2_{B-β}), 50.9 (C-2_{B-α}), 17.2 (C-6_{B}). HRMS (ESI⁺): m/z [M+NH₄]⁺ calcd for C₃₁H₃₇Cl₆N₆O₁₀, 863.0697; found 863.0700.

(Benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl (N-phenyl)trifluoroacetimidate (39b) and 2-trichloromethyl [(benzyl 3-O-benzyl-2-deoxy-4-O-methyl-2-trichloroacetamido-α-L-altropyranosyluronate)-(1→3)-4-azido-1,2,4,6-tetradeoxy-α-D-galactopyrano]-[2,1,d]-oxazoline (40b). Hemiacetal 38b (3.0 g, 3.55 mmol, 1.0 equiv.) was dissolved in acetone (40 mL). PTFACl (731 μL, 4.61 mmol, 1.3 equiv.) was added followed by Cs₂CO₃ (1.27 g, 3.90 mmol, 1.1 equiv.). After stirring for 2 h at rt, the reaction mixture was filtered, washed with acetone (2*20 mL) and the filtrate was concentrated under reduced pressure. Flash chromatography of the crude material eluting with cHex/EtOAc/Et₃N (20:1:0.2) gave a mixture of the (N-phenyl)trifluoroacetimidate donor 39b and a minor amount of oxazoline 40b (3.0 g, 2.95 mmol, 83% based on 39b) as a white solid. The disaccharide donor 39b had R_{f} 0.7 (Tol/EtOAc 4:1); HRMS (ESI⁺): m/z [M+NH₄]⁺ calcd for C₃₉H₄₁Cl₆F₃N₇O₁₀, 1034.0970; found 1034.0993. Oxazoline 40b R_{f} 0.5 (Tol/EtOAc 4:1); HRMS (ESI⁺): m/z [M+H]⁺ calcd for C₃₁H₃₂Cl₆N₅O₉, 828.0326; found 828.0305.

Oligosaccharides Equipped with a 4_{A}-Endchain Methyl Group: Chain Elongation

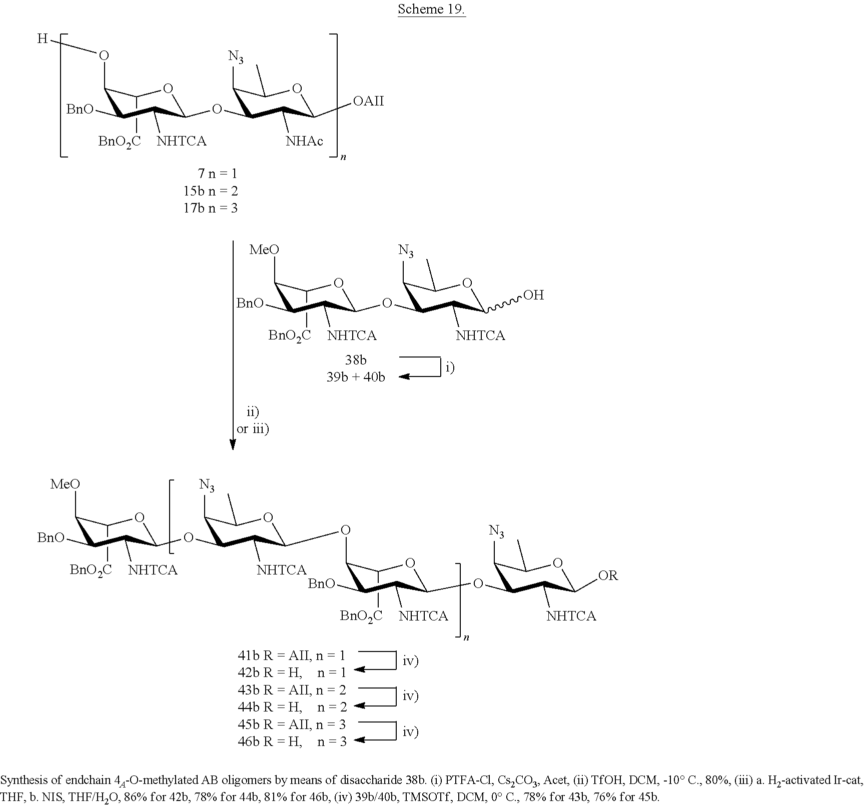

Allyl (benzyl 3-O-benzyl-2-deoxy-4-O-methyl-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (41b). PTFACl (110 μL, 692 μmol, 1.3 equiv.) and Cs₂CO₃ (191 mg, 586 μmol, 1.1 equiv.) were added to hemiacetal 38b (450 mg, 533 μmol, 1.0 equiv.) in acetone (10 mL). After stirring for 2 h at rt, the reaction mixture was passed through pad of Celite and solids were washed with acetone. The filtrate was concentrated and dried under vacuum to give the crude mix of donors 39b/40b (540 mg, quant.).

A mix of crude donors 39b/40b (540 mg, 533 μmol, 1.0 equiv. theo.) and acceptor 7 (464 mg, 533 μmol, 1.0 equiv.) was coevaporated with anhyd toluene (10 mL) and dried under vacuum for 1 h. 4 Å MS (1 g) was added to a solution of the later in anhyd. DCM (20 mL) and the suspension was stirred for 45 min under an Ar atmosphere at rt. After cooling to −10° C., TfOH (2.4 μL, 27 μmol, 0.05 equiv.) was added and stirring was continued for 30 min while the bath temperature was kept at 0° C. A TLC analysis (Tol/EtOAc 4:1) showed the absence of donor 39b/40b and the presence of a new spot. At completion, Et₃N (5 μL) was added. The suspension was filtered through a fitted funnel and solids were washed with DCM (10 mL) twice. Volatiles were evaporated and the residue was purified by flash chromatography (cHex/EtOAc 85:15→75:25) to give tetrasaccharide 41b as a white solid (730 mg, 0.429 mmol, 80%). The coupling product 41b had R_{f} 0.55 (Tol/EtOAc 7:3). ¹H NMR (CDCl₃) δ 7.45-7.17 (m, 20H, H_{Ar}), 6.98 (d, 1H, J_{2,NH}=6.8 Hz, NH_{B}), 6.77 (d, 1H, J_{2,NH}=7.2 Hz, NH_{B}), 6.64 (d, 1H, J_{2,NH}=8.0 Hz, NH_{A}), 6.49 (d, 1H, J_{2,NH}=6.4 Hz, NH_{A}), 5.89-5.80 (m, 1H, CH_{All}), 5.33-5.14 (mₚₒ, 6H, CH_{2Bn-6}, CH_{2All}), 5.16 (dₚₒ, 1H, J_{1,2}=5.6 Hz, H-1_{A}), 5.07 (dₚₒ, 1H, J_{1,2}=5.6 Hz, H-1_{A}), 4.83 (d, 1H, J_{1,2}=8.4 Hz, H-1_{B}), 4.79 (dₚₒ, 1H, J_{1,2}=8.4 Hz, H-1_{B}), 4.77 (dₚₒ, 1H, J_{4,5}=5.2 Hz, H-5_{A}), 4.71-4.64 (m, 4H, H-3_{B}, H-3_{B1}, H-4_{A}, H-5_{A}) 4.54 (d, 1H, CH_{2Bn}), 4.45 (brs, 2H, CH_{2Bn}), 4.33-4.28 (m, 1H, CH_{2All}), 4.23 (brs, 1H, H-4_{A1}) 4.21-4.16 (m, 1H, H-2_{A}), 4.06-4.01 (m, 1H, CH_{2All}), 3.92 (d, 1H, J_{3,4}=3.2 Hz, H-4_{B}), 3.90-3.84 (m, 4H, H-2_{A1}, H-3_{A}, H-3_{A1}, H-4_{A1}), 3.78 (d, 1H, J_{3,4}=3.2 Hz, H-4_{B1}), 3.62-3.57 (m, 2H, H-2_{B}, H-5_{B}), 3.50-3.43 (m, 2H, H-2_{B}, H-5_{B}), 3.39 (s, 3H, OCH₃), 1.30 (d, 3H, J_{5,6}=6.0 Hz, H-6_{B}), 1.19 (d, 3H, J_{5,6}=6.0 Hz, H-6_{B}). ¹³C NMR (CDCl₃) δ 168.8, 168.2 (CO_{CO2Bn}), 162.0, 161.9, 161.6 (CO_{NHTCA}), 137.4, 137.1, 135.1, 135.0 (C_{q,Ar}), 133.5 (CH_{All}), 129.1, 129.0, 128.9, 128.7 (2C), 128.6, 128.5, 128.4, 128.3, 128.2, 128.0, 125.2 (C_{Ar}), 117.9 (CH_{2All}), 99.0 (C-1_{A1}, ¹J_{C,H}=170.8 Hz), 98.9 (C-1_{B1}, ¹J_{C,H}=165.7 Hz), 98.1 (C-1_{A}, ¹J_{C,H}=170.4 Hz), 97.8 (C-1_{B}, ¹J_{C,H}=162.3 Hz), 92.4, 92.3 (2C), 92.2 (CCl₃), 75.0 (3C), 74.7 (C-5_{A}, C-4_{A}, C-3_{B}, C-3_{B1}), 73.2, 73.0, (C-3_{A}, C-3_{A1}), 72.5 (CH_{2Bn}), 72.0 (CH_{2Bn}), 71.9 (C-4_{A1}), 71.5 (C-5_{A1}), 70.0 (CH_{2All}), 69.3, 68.8 (C-5_{B}, C-5_{B1}), 67.6, 67.4 (CH_{2Bn-6}), 65.5, 65.1 (C-4_{B}, C-4_{B1}), 58.1 (OCH₃), 55.6, 55.5 (C-2_{B}, C-2_{B1}), 54.1, 52.9 (C-2_{A}, C-2_{A1}), 17.3, 17.2 (C-6_{B}, C-6_{B1}). HRMS (ESI⁺): m/z [M+NH₄]⁺ calcd for C₃₄H₇₀Cl₁₂N₁₁O₁₉ 1716.1106, found 1716.1147.

(Benzyl 3-O-benzyl-4-O-methyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-α-D-galactopyranose (42b). [Ir(COD)(PMePh₂)₂]⁺PF₆⁻ (16 mg, 19 μmol, 0.05 equiv.) was dissolved in anhyd. THF (3.0 mL) and stirred for 30 min under an H₂ atmosphere. The resulting yellow solution was degassed repeatedly and transferred into a solution of allyl glycoside 41b (650 mg, 0.383 mmol, 1.0 equiv.) in anhyd. THF (12 mL). After stirring for 2 h at rt, NIS (103 mg, 0.459 mmol, 1.2 equiv.) was added. After stirring for another 2 h at rt, the reaction was quenched with 10% aq. sodium sulphite. Volatiles were evaporated and the aq. phase was extracted with DCM (3×15 mL). The organic phases were pooled, washed with brine, dried over anhyd. Na₂SO₄ and concentrated. Purification by flash chromatography (Tol/EtOAc 70:30→65:35) gave hemiacetal 42b (α/β 5:1, 550 mg, 331 μmol, 86%) as a white foam. The α/β-anomer had R_{f} 0.35, 0.55 (Tol/EtOAc 7:3). The α-anomer had ¹H NMR (CDCl₃) δ 7.45-7.17 (m, 20H, H_{Ar}), 7.01 (d, 1H, J_{2,NH}=6.8 Hz, NH_{B}), 6.77 (d, 1H, J_{2,NH}=7.2 Hz, NH_{B}), 6.64 (d, 1H, J_{2,NH}=8.0 Hz, NH_{A}), 6.49 (d, 1H, J_{2,NH}=6.4 Hz, NH_{A}), 5.33-5.18 (m, 4H, CH_{2Bn-6}), 5.08 (d, 1H, J_{1,2}=7.2 Hz, H-1_{A}), 5.04 (d, 1H, J_{1,2}=5.6 Hz, H-1_{A1}), 4.87 (dₚₒ, 1H, J_{1,2}=8.0 Hz, H-11), 4.85 (ddₚₒ, 1H, H-1_{B}), 4.76 (dₚₒ, 1H, J_{4,5}=5.0 Hz, H-5_{A}), 4.71 (ddₚₒ, 1H, J_{2,3}=10.8 Hz, J_{3,4}=3.6 Hz, H-3_{B}), 4.68-4.63 (mₚₒ, 3H, CH_{2Bn}), 4.56 (d, 1H, J=12.0 Hz, CH_{2Bn}), 4.48 (dd, 2H, J=12.4 Hz, CH_{2Bn}), 4.23-4.17 (m, 2H, H-2_{A}, H-4_{A}), 3.92-3.81 (m, 5H, H-2_{A}, H-3_{A}, H-3_{A1}, H-4_{B}), 3.78 (brd, 1H, J_{3,4}=3.6 Hz, H-4_{B1}), 3.35-3.42 (m, 4H, H-2_{B}, H-2_{B1}, H-5_{B}, H-5_{B1}), 3.39 (s, 3H, OCH₃), 1.22, 1.18 (2d, 6H, J_{5,6}=6.0 Hz, H-6_{B1}, H-6_{B1}). ¹³C NMR (CDCl₃) δ 168.9, 168.2 (2C, C-6_{A}, C-6_{A1}), 162.3, 162.0, 161.6 (2C) (CO_{NHTCA}), 137.4, 137.0, 135.1, 135.0 (C_{q,Ar}), 129.1, 129.0, 128.7 (2C), 128.6, 128.4 (2C), 128.3, 128.2, 128.0, 125.2 (C_{Ar}), 99.1, 98.8, 98.2, 96.7, 92.4, 92.3, (C-1), 92.2 (2C, CCl₃), 77.3 (CH_{2Bn}), 77.2, 75.0 (3C, C-5_{A}, C-4_{A}, C-3_{B}), 74.4 (C-3_{B1}), 73.1, 72.9 (2C, C-3_{A}, C-3_{A1}), 72.0 (CH_{2Bn}), 71.9 (C-4_{A1}), 71.4 (C-5_{A1}), 69.2, 68.9 (2C, C-5_{B}, C-5_{B1}), 67.5, 67.4 (CH_{2Bn-6}), 65.1 (2C, C-4_{B}, C-4_{B1}), 58.1 (OCH₃), 55.5, 55.1 (2C, C-2_{B}, C-2_{B1}), 53.9, 52.8 (2C, C-2_{A}, C-2_{A1}), 17.4, 17.2 (2C, C-6_{B}, C-6_{B1}). HRMS (ESI⁺): m/z [M+NH₄]⁺ calcd for C₆₁H₆₆Cl₁₂N₁₁O₁₉ 1676.0793, found 1676.0797.

Allyl (benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (43b). [2+4]-Glycosylation: PTFACl (73 μL, 462 μmol, 1.3 equiv.) and Cs₂CO₃ (127 mg, 391 μmol, 1.1 equiv.) were added to hemiacetal 38b (300 mg, 355 μmol, 1.0 equiv.) in acetone (10 mL). After stirring for 2 h at rt, the reaction mixture was filtered through Celite bed and washed with acetone (5 mL) twice. The filtrate was concentrated under reduced pressure to give the crude donors 39b/40b (˜360 mg, 0.355 mmol, quant.), which was used as such in the next step after extensive drying under high vacuum.

A mix of donors 39b/40b (355 μmol, 1.0 equiv. theo.) and acceptor 15b (598 mg, 355 μmol, 1.0 equiv.) were coevaporated with anhyd. toluene (10 mL) twice and then dried extensively under high vacuum. The mixture was stirred with freshly activated MS 4 Å (1.0 g) in anhyd. DCM (12 mL) for 45 min under an Ar atmosphere at rt. After cooling to 0° C., TMSOTf (3.2 μL, 18 μmol, 0.05 equiv.) was added and stirring was continued for 50 min while keeping the bath temperature at 0° C. A TLC analysis (Tol/EtOAc 6:4) showed the absence of donors 39b/40b and the presence of a new spot. At completion, Et₃N (3 μL) was added. The suspension was passed through a fitted funnel and solids were washed with DCM (5 mL) twice. Volatiles were evaporated and the residue was purified by flash chromatography (Tol/ACN 82:18→85:15) to give hexasaccharide 43b as a white solid (700 mg, 278 μmol, 78%). The coupling product 43b had R_{f} 0.4 (Tol/ACN 4:1). HRMS (ESI⁺): m/z [M+2NH₄]²⁺ calcd for C₉₄H₁₀₃Cl₁₈N₁₇O₂₈ 1273.5770; found 1273.5784.

(Benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranose (44b). [Ir(COD)(PMePh₂)₂]⁺PF₆⁻ (12 mg, 14 μmol, 0.05 equiv.) was dissolved in anhyd. THF (4 mL) and the solution was stirred for 20 min under an H₂ atmosphere. The resulting yellow solution was degassed repeatedly and poured into a solution of allyl glycoside 43b (700 mg, 278 μmol, 1.0 equiv.) in anhyd. THF (6 mL). After stirring for 2 h at rt, NIS (94 mg, 418 μmol, 1.5 equiv.) was added. After stirring for another 2 h at rt, the reaction was quenched with 10% aq sodium sulphite. Volatiles were eliminated under vacuum and the aq. phase was extracted repeatedly with DCM (10 mL). The organic layers were pooled, washed with brine, dried over anhyd. Na₂SO₄ and concentrated. Purification by flash chromatography (Tol/EtOAc 75:25→65:35) gave hemiacetal 44b (α/β mixture, 540 mg, 219 μmol, 78%) as a white foam, R_{f} 0.25, 0.4 (Tol/EtOAc 7:3).

Allyl (benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (45b). [2+6]-Glycosylation: A mix of the PTFA donor donors 39b/40b (200 mg, 0.197 mmol, 1.4 equiv.) and acceptor 17b (344 mg, 0.138 mmol, 1.0 equiv.) were stirred with freshly activated MS 4 Å (600 mg) in anhyd. DCM (6 mL) for 30 min under an Ar atmosphere at rt. After cooling to −10° C., TMSOTf (1.8 μL, 10 μmol, 0.05 equiv.) was added and stirring was continued for 1 h while keeping the bath temperature at −10° C. At completion, Et₃N (3 μL) was added. The suspension was filtered through a fitted funnel and washed with DCM (6 mL) twice. Volatiles were evaporated and the residue was purified by flash chromatography (Tol/ACN 85:15→82:18) to give octasaccharide 45b as a white solid (350 mg, 0.105 mmol, 76%). The coupling product 45b had R_{f} 0.35 (Tol/ACN 4:1). HRMS (ESI⁺): m/z [M+NH₄]⁺ calcd for C₁₂₄H₁₂₈Cl₂₄N₂₁O₃₇ 3356.1160; found 3356.1186.

(Benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranose (46b). [Ir(COD)(PMePh₂)₂]⁺PF₆⁻ (4.0 mg, 5 μmol, 0.05 equiv.) was dissolved in anhyd THF (2.0 mL) and stirred for 40 min under an H₂ atmosphere. The resulting yellow solution was degassed with a flow of Ar and poured into a solution of octasaccharide 45b (700 mg, 0.105 mmol, 1.0 equiv.) in anhyd. THF (3.0 mL). After stirring for 2 h at rt, NIS (31 mg, 317 μmol, 1.3 equiv.) was added. After stirring for another 2 h at rt, the reaction was quenched by adding 10% aq. sodium sulphite. The reaction mixture was concentrated and the aq. phase was extracted with DCM (5 mL) three times. The organic phases were pooled, washed with brine, dried over anhyd. Na₂SO₄ and concentrated. Purification by flash chromatography (Tol/EtOAc 70:30→60:40) gave the corresponding hemiacetal 46b (α/β mixture, 280 mg, 85 μmol, 81%) as a white foam. Hemiacetal 46b had R_{f} 0.15, 0.25 (Tol/EtOAc 6:4). HRMS (ESI⁺): m/z [M+2NH₄]²⁺ calcd for C₁₂₁H₁₂₈Cl₂₄N₂₂O₃₇ 1662.0643; found 1662.0621.

### Example 8. Linker-Equipped Oligosaccharides Featuring a 4_{A}-Me at the Endchain A Residue: Linker=(S)-(−)-2,3-dibenzyloxy-1-propanol

Linker Attachment

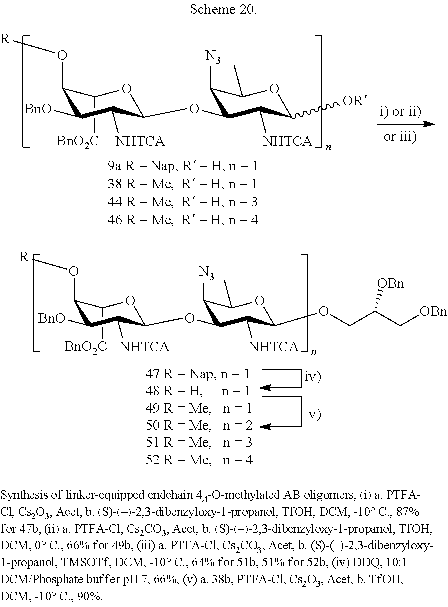

(S)-2,3-Dibenzyloxy-1-propyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranoside (47b). PTFACl (212 μL, 1.33 mmol, 1.3 equiv.) and Cs₂CO₃ (369 mg, 1.13 mmol, 1.1 equiv.) were added to hemiacetal 9a (1.0 g, 1.03 mmol, 1.0 equiv.) in acetone (20 mL). After stirring for 2 h at rt, the reaction mixture was filtered through a pad of Celite and solids were washed with acetone (10 mL) twice. The filtrate was concentrated and dried under vacuum to give the crude PTFA/oxazoline donors 9b/10b (600 mg, 0.592 mmol, quant.), which was used as such in the next step after extensive drying under vacuum.

A mix of the crude donors 9b/10b (1.17 g, 1.03 mmol, 1.0 equiv. theo.) and (S)-(−)-2,3-dibenzyloxy-1-propanol (519 μL, 2.06 mmol, 2.0 equiv.) was stirred with freshly activated MS 4_{A} (1 g) in anhyd. DCM (20 mL) for 1 h under an Ar atmosphere at rt. After cooling to −10° C., TfOH (4.5 μL, 51 μmol, 0.05 equiv.) was added and stirring was continued for 30 min during which the bath temperature was kept at −10° C. A TLC analysis (Tol/EtOAc 4:1) showed the absence of donors 9b/10b and the presence of a new spot. At completion, Et₃N (10 μL) was added. The suspension was filtered through a fitted funnel and washed with DCM (20 mL) twice. Volatiles were evaporated and the residue was purified by flash chromatography (cHex/EtOAc 80:20→76:24) to give disaccharide 47b as a white solid (1.1 g, 0.897 mmol, 87%). The coupling product 47b had R_{f} 0.6 (Tol/EtOAc 4:1). ¹H NMR (CDCl₃) δ 7.85-7.74 (m, 3H, H_{Ar}), 7.70 (brs, 1H, H_{Ar}), 7.45-7.48 (m, 2H, H_{Ar}), 7.45-7.48 (m, 21H, H_{Ar}), 6.71 (d, 1H, J_{2,NH}=7.2 Hz, NH_{A}), 6.65 (d, 1H, J_{2,NH}=7.8 Hz, NH_{B}), 5.23 (brs, 2H, CH_{2Bn}), 5.18 (d, 1H, J_{1,2}=5.2 Hz, H-1_{A}), 4.86 (d, 1H, J_{4,5}=5.2 Hz, H-5_{A}), 4.73 (brs, 2H, CH_{2Bn}), 4.73 (dₚₒ, 1H, J_{1,2}=8.0 Hz, H-1_{B}), 4.66 (brs, 2H, CH_{2Bn}), 4.58-4.54 (m, 3H, CH_{2Bn}), 4.49-4.45 (mₚₒ, 3H, CH₂₃n, H-3_{B}), 4.22 (dddₚₒ, 1H, H-2_{A}), 4.08 (dd, 1H, J_{3,4}=2.8 Hz, H-4_{A}), 3.96 (dd, 1H, J=4.4 Hz, 10.4 Hz, H₂₋ₗᵢₙₖₑᵣ), 3.85-3.82 (m, 2H, H-3_{A}, H-4_{B}), 3.78-3.73 (m, 1H, H₁₋ₗᵢₙₖₑᵣ), 3.67-3.52 (m, 5H, H₁₋ₗᵢₙₖₑᵣ, H₃₋ₗᵢₙₖₑᵣ, H-2_{B}, H-5_{B}), 1.27 (d, 3H, J_{5,6}=6.4 Hz, H-6_{B}). ¹³C NMR (CDCl₃) δ 168.9 (C-6_{A}), 161.9, 161.6 (CO_{NHTCA}), 138.5, 138.3, 137.4, 135.0, 133.1 (2C) (C_{q,Ar}), 128.7, 128.6, 128.4, 128.3 (2C), 128.2, 127.9 (2C), 127.7 (2C), 127.6, 127.5 (2C), 127.0, 126.2, 126.1, 125.9 (C_{Ar}), 99.4 (C-1_{B}, ¹J_{C,H}=163.4 Hz), 98.9 (C-1_{A}, ¹J_{C,H}=171.0 Hz), 92.3, 92.1 (2C, CCl₃), 77.0 (CH), 76.3 (C-3_{B}), 73.4 (C-3_{A}), 73.3 (CH_{2Bn}), 72.1 (CH_{2Nap}), 72.0 (2C, C-4_{A}, CH_{2Bn}), 71.9 (CH_{2Bn}), 71.6 (C-5_{A}), 70.1 (CH_{2Bn}), 69.4 (C-5_{B}), 68.8 (CH_{2Bn}), 67.5 (CH_{2Bn-6}), 65.1 (C-4_{B}), 55.2 (C-2_{B}), 53.0 (C-2_{A}), 17.3 (C-6_{B}). HRMS (ESI⁺): m/z [M+NH₄]⁺ calcd for C₅₈H₆₁Cl₆N₆O₂ 1243.2473; found 1243.2493.

(S)-2,3-Dibenzyloxy-1-propyl (benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (48b). DDQ (482 mg, 2.12 mmol, 2.6 equiv.) was added to disaccharide 47b (1.0 g, 816 μmol, 1.0 equiv.) in a mix of DCM (20.0 mL) and phosphate buffer pH 7 (2.0 mL). The biphasic mixture was cooled to 0° C. and stirred vigorously for 2 h. A TLC analysis (Tol/EtOAC 4:1) showed the absence of the fully protected 47b and the presence of a more polar spot. 10% Aq. NaHCO₃ (20 mL) was added followed by DCM (20 mL). The DCM layer was separated, washed with water and brine, dried over Na₂SO₄, filtered, and concentrated under reduced pressure. The residue was purified by flash chromatography (Tol/EtOAc 80:20→75:25) to give alcohol 48b (585 mg, 539 μmol, 66%) as a white solid. Disaccharide 48b had R_{f} 0.5 (Tol/EtOAc 4:1). ¹H NMR (CDCl₃) δ 7.43-7.26 (m, 20H, H_{Ar}), 6.80 (d, 1H, J_{2,NH}=8.2 Hz, NH_{A}), 6.73 (d, 1H, J_{2,NH}=7.2 Hz, NH_{B}), 5.26 (ddₚₒ, 2H, J=12.0 Hz, CH_{2Bn}), 4.96 (d, 1H, J_{1,2}=7.6 Hz, H-1_{A}), 4.76 (dₚₒ, 1H, J_{1,2}=8.2 Hz, H-1_{B}), 4.75-4.68 (mₚₒ, 3H, H-5_{A}, CH_{2Bn}), 4.65 (ddₚₒ, 2H, CH_{2Bn}), 4.53 (ddₚₒ, 2H, CH_{2Bn}), 4.47 (dd, 1H, J_{2,3}=10.6 Hz, J_{3,4}=3.6 Hz, H-3_{B}), 4.34-4.29 (mₚₒ, 1H, H-2_{A}), 4.08 (dt, 1H, J_{4,5}=7.6 Hz, J_{3,4}=3.6 Hz, H-4_{A}), 3.98 (dd, 1H, J=3.6, 5.6 Hz, H₃₋ₗᵢₙₖₑᵣ), 3.84 (dd, 1H, J=3.6, 5.6 Hz, H-3_{A}), 3.77-3.72 (m, 2H, H-4_{B}, H₂₋ₗᵢₙₖₑᵣ), 2.82 (d, 1H, J_{4,OH}=8.0 Hz, OH), 3.68-3.55 (m, 5H, H₁₋ₗᵢₙₖₑᵣ, H₃₋ₗᵢₙₖₑᵣ, H-2_{B}, H-5_{B}), 1.27 (d, 3H, J_{5,6}=6.4 Hz, H-6_{B}). ¹³C NMR (CDCl₃) δ 168.8 (C-6_{A}), 162.1, 161.6 (CO_{NHTCA}), 138.4, 138.2, 137.1, 134.9 (C_{q,Ar}), 128.7, 128.5, 128.4 (2C), 128.3 (2C), 128.2, 127.6 (2C) (C_{Ar}), 99.5 (C-1_{A}, ¹J_{C,H}=171.2 Hz), 99.3 (C-1_{B}, ¹J_{C,H}=163.4 Hz), 92.2, 91.9 (2C, CCl₃), 77.0 (CH), 76.7 (C-3_{B}), 74.7 (C-3_{A}), 73.3 (CH_{2Bn}), 72.2 (CH_{2Bn}), 72.1 (CH_{2Bn}), 71.2 (C-5_{A}), 70.0 (CH_{2Bn}), 69.6 (C-5_{B}), 68.8 (CH_{2Bn}), 67.5 (CH_{2Bn-6}), 65.5 (C-4_{A}), 65.3 (C-4_{B}), 55.4 (C-2_{B}), 51.3 (C-2_{A}), 17.2 (C-6_{B}). HRMS (ESI⁺): m/z [M+NH₄]⁺ calcd for C₄₇H₅₃Cl₆N₆O₁₂, 1103.1858; found 1103.1847.

(S)-2,3-Dibenzyloxy-1-propyl (benzyl 3-O-benzyl-2-deoxy-4-O-methyl-2-trichloroacetamido-α-L-altropyranosyluronate)-(1→3)-4-azido-2-trichloroacetamido-2,4,6-trideoxy-β-D-galactopyranoside (49b). PTFACl (122 μL, 770 μmol, 1.3 equiv.) and Cs₂CO₃ (212 mg, 651 μmol, 1.1 equiv.) were added to hemiacetal 38b (500 mg, 592 μmol, 1.0 equiv.) in acetone (15 mL). After stirring for 2 h at rt, the reaction mixture was filtered through a pad of Celite bed and solids were washed with acetone. Volatiles were evaporated to give a mix of the crude donors 39b/40b (600 mg, quant.), which was used as such in the next step after extensive drying under vacuum.

A mix of the crude donors 39b/40b (600 mg, 592 μmol, 1.0 equiv. theo.) and (S)-(−)-2,3-dibenzyloxy-1-propanol (298 μL, 1.18 mmol, 2.0 equiv.) was stirred with freshly activated 4_{A} MS (500 mg) in anhyd. DCM (20 mL) for 1 h under an Ar atmosphere at rt. After cooling to 0° C., TfOH (2.6 μL, 30 μmol, 0.05 equiv.) was added and stirring was continued at this temperature for 40 min. A TLC analysis (Tol/EtOAc 4:1) showed the absence of donors 39b/40b and the presence of a new spot. At completion, Et₃N (5 μL) was added. The suspension was passed through a fitted funnel and solids were washed with DCM (10 mL) twice. Volatiles were evaporated and the residue was purified by flash chromatography (cHex/EtOAc 80:20→76:24) to give disaccharide 49b as a white solid (430 mg, 423 μmol, 66%). The coupling product 49b had R_{f} 0.8 (Tol/EtOAc 4:1). ¹H NMR (CDCl₃) δ 7.42-7.26 (m, 20H, H_{Ar}), 6.74 (ddₚₒ, 2H, J_{2,NH}=7.6 Hz, NH_{A}, NH_{B}), 5.26 (brd, 2H, CH_{2Bn-6}), 5.20 (d, 1H, J_{1,2}=5.6 Hz, H-1_{A}), 4.79 (d, 1H, J_{4,5}=4.8 Hz, H-5_{A}), 4.73 (d, 1H, J_{1,2}=8.4 Hz, H-1_{B}), 4.66-4.63 (m, 3H, CH_{2Bn}), 4.57-4.53 (m, 3H, CH_{2Bn}), 4.49 (dd, 1H, J_{2,3}=10.8 Hz, J_{3,4}=3.6 Hz, H-3_{B}), 4.15-4.10 (m, 1H, H-2_{A}), 3.97-3.92 (m, 2H, H-3_{A}, OCH₂), 3.86-3.82 (m, 2H, H-4_{B}, H-4_{A}), 3.76-3.72 (m, 1H, CH), 3.67-3.54 (m, 5H, H-2_{B}, H-5_{B}, 3OCH₂), 3.39 (s, 3H, OCH₃), 1.26 (d, 3H, J_{5,6}=6.0 Hz, H-6_{B}). ¹³C NMR (CDCl₃) δ 168.9 (C-6_{A}), 161.9, 161.7 (CO_{NHTCA}), 138.5, 138.3, 137.4, 135.0 (C_{q,Ar}), 128.8, 128.7 (2C), 128.4, 128.3 (2C), 128.2, 128.0, 127.6, 127.5 (3C) (C_{Ar}), 99.4 (C-1_{B}, ¹J_{C,H}=163 Hz), 98.6 (C-1_{A}, ¹J_{C,H}=169 Hz), 92.3, 92.2 (2C, CCl₃), 77.0 (CH), 76.2 (C-3_{B}), 75.1 (C-4_{A}), 73.3 (CH_{2Bn}), 73.1 (C-3_{A}), 72.1 (CH_{2Bn}), 72.0 (CH_{2Bn}), 71.4 (C-5_{A}), 70.1 (OCH₂), 69.4 (C-5_{B}), 68.8 (OCH₂), 67.5 (CH_{2Bn-6}), 65.1 (C-4_{B}), 58.0 (OCH₃), 55.2 (C-2_{B}), 53.2 (C-2_{A}), 17.2 (C-6_{B}). HRMS (ESI⁺): m/z [M+NH₄]⁺ calcd for C₄₈H₅₅Cl₆N₆O₁₂, 1117.2004; found 1117.1999.

(S)-2,3-Dibenzyloxy-1-propyl (benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (50b). PTFA-Cl (73 μL, 0.460 mmol, 1.3 equiv.) and Cs₂CO₃ (127 mg, 385 μmol, 1.1 equiv.) were added to hemiacetal 38b (300 mg, 353 μmol, 1.0 equiv.) in acetone (10 mL). After stirring for 2 h at rt, the reaction mixture was filtered through a pad of Celite and washed with acetone (5 mL) twice. The filtrate was concentrated under reduced pressure to give the crude PTFA donor (360 mg, quant.), which was used as such in the next step after extensive drying under high vacuum.

A mix of the crude PTFA donor (360 mg, 0.355 mmol, 1.25 equiv. theo.) and acceptor 48b (308 mg, 284 μmol, 1.0 equiv.) were coevaporated with anhyd. toluene (5 mL) twice and then dried extensively under high vacuum. Freshly activated 4 Å MS (600 mg) was added to the mixture in anhyd. DCM (9.0 mL) and the suspension was stirred for 1 h under an Ar atmosphere at rt. After cooling to −10° C., TfOH (1.6 μL, 18 μmol, 0.06 equiv.) was added and stirring was continued for 30 min while keeping the bath temperature at 0° C. A TLC analysis (Tol/EtOAc 14:6) showed the absence of donor and the presence of a new spot. At completion, Et₃N (3 μL) was added. The suspension was passed through a fitted funnel and solids were washed with DCM (5 mL) twice. Volatiles were evaporated and the residue was purified by flash chromatography (cHex/EtOAc 82:18→77:23) to give tetrasaccharide 50b as a white solid (490 mg, 256 μmol, 90%). The coupling product 50b had R_{f} 0.7 (Tol/EtOAc 7:3). HRMS (ESI⁺): m/z [M+NH₄]⁺ calcd for C₇₈H₈₄Cl₁₂N₁₁O₂₁ 1930.2100; found 1930.2138.

(S)-2,3-Dibenzyloxy-1-propyl (benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (51b). Hemiacetal 44b (540 mg, 219 μmol) was dissolved in acetone (6 mL). PTFA-Cl (57 μL, 357 μmol, 1.6 equiv.) and Cs₂CO₃ (102 mg, 0.313 mmol, 1.4 equiv.) were added. After stirring for 2 h at rt, the suspension was passed through a pad of Celite and solids were washed with acetone (5 mL) twice. Volatiles were evaporated to give the crude PTFA donor (577 mg, quant.), which was used as such in the next step after extensive drying under vacuum.

A mix of the crude donor (577 mg, 219 μmol, 1.0 equiv. theo.) and (S)-(−)-2,3-dibenzyloxy-1-propanol (169 μL, 670 μmol, 3.0 equiv.) in anhyd. DCM (12 mL) containing freshly activated MS 4 Å (1.2 g) was stirred for 30 min under an Ar atmosphere at rt. After cooling to −10° C., TMSOTf (2.0 μL, 11 μmol, 0.05 equiv.) was added and stirring was continued for 40 min while keeping the bath temperature at −10° C. At completion, Et₃N (4 μL) was added. The suspension was filtered through a fitted funnel and washed with DCM (6 mL) twice. Volatiles were evaporated and the residue was purified by flash chromatography (Tol/ACN 82:18→85:15) to give hexasaccharide 51b as a white solid (380 mg, 139 μmol, 64%). The coupling product 51b had R_{f} 0.45 (Tol/ACN 4:1). HRMS (ESI⁺): m/z [M+NH₄]⁺ calcd for C₁₀₈H₁₁₈Cl₁₈N₁₅O₃₀ 2243.2255; found 2746.2190.

(S)-2,3-Dibenzyloxy-1-propyl (benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-4-O-methyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (52b). PTFACl (18 μL, 111 μmol, 1.3 equiv.) and Cs₂CO₃ (31 mg, 94 μmol, 1.1 equiv.) were added to a solution of hemiacetal 46b (280 mg, 85 μmol, 1.0 equiv.) in acetone (5.0 mL). After stirring for 2 h at rt, the reaction mixture was passed through a pad of Celite and solids were washed with acetone (3 mL) twice. The filtrate was concentrated under reduced pressure to give the crude donor (300 mg, quant.), which was used as such in the next step after extensive drying under vacuum.

A mix of the crude PTFA donor (300 mg, 85 μmol, 1.0 equiv. theo.) and (S)-(−)-2,3-dibenzyloxy-1-propanol (85 μL, 341 μmol, 4.0 equiv.) in anhyd. DCM (6.0 mL) containing freshly activated MS 4 Å (300 mg) was stirred for 30 min under an Ar atmosphere at rt. After cooling to −10° C., TMSOTf (1.0 μL, 4 μmol, 0.05 equiv.) was added and stirring was continued for 45 min while keeping the temperature of the bath at −10° C. At completion, Et₃N (2 μL) was added. After stirring at this temperature for another 10 min, the suspension was passed through a fitted funnel and solids were washed with DCM (4 mL) twice. Volatiles were evaporated and the residue was purified by flash chromatography (Tol/ACN 84:16→78:22) to give octasaccharide 52b as a white solid (155 mg, 44 mmol, 51%). The condensation product 52b had R_{f} 0.6 (Tol/ACN 4:1). HRMS (ESI⁺): m/z [M+2NH₄]²⁺ calcd for C₁₃₈H₁₄₆Cl₂₄N₂₂O₃₉ 1793.1260; found 1793.1251.

Full Deprotection

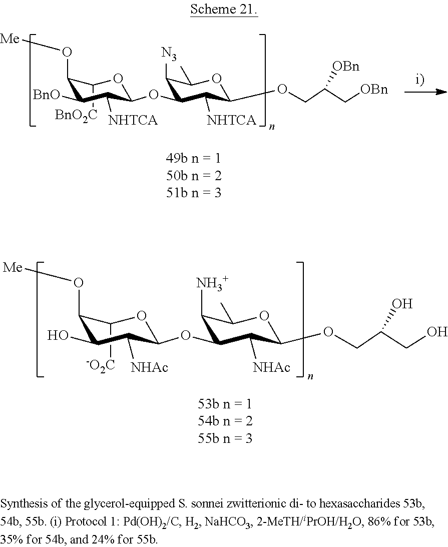

(S)-2,3-Dihydroxy-1-propyl (2-acetamido-2-deoxy-α-L-altropyranosyluronic acid)-(1→3)-2-acetamido-4-amino-2,4,6-trideoxy-β-D-galactopyranoside (53b). The fully protected disaccharide 49b (120 mg, 109 μmol) was subjected to hydrogenation-mediated deprotection (protocol 1). Disaccharide 53b was obtained as a white solid (48 mg, 94 μmol, 86%). Disaccharide 53b had RP-HPLC (215 nm) Rₜ=12.3 min (conditions A), Rₜ=6.3 min (conditions B). ¹H NMR (D₂O) δ 4.88 (d, 1H, J_{1,2}=8.3 Hz, H-1_{A}), 4.78 (brs, 1H, H-5_{A}), 4.48 (d, 1H, J_{1,2}=8.6 Hz, H-1_{B}), 4.13 (dd, 1H, J_{3,4}=4.4 Hz, J_{2,3}=10.8 Hz, H-3_{B}), 4.02 (qₚₒ, 1H, H-5_{B}), 3.98 brt, 1H, H-4_{A}), 3.90 (ddₚₒ, 1H, H-2_{A}), 3.85 (brd, 1H, H-4_{B}), 3.84-3.74 (mₚₒ, 3H, H-2_{B}, H₁₋ₗᵢₙₖₑᵣ), 3.70 (dd, 1H, J_{2,3}=10.8 Hz, J_{3,4}=2.0 Hz, H-3_{A}), 3.62-3.55 (m, 2H, H₁₋ₗᵢₙₖₑᵣ), 3.49-3.45 (mₚₒ, 1H, H₂₋ₗᵢₙₖₑᵣ), 3.48 (brs, 3H, OCH₃), 2.00, 1.96 (2s, 6H, CH_{3Ac}), 1.29 (d, 3H, J_{5,6}=6.6 Hz, H-6_{B}). ¹³C NMR (D₂O) δ 174.5, 174.2 (NHCO_{A,B}), 172.7 (C-6_{A}), 101.7 (C-1_{B}, ¹J_{C,H}=163.0 Hz), 100.8 (C-1_{A}, ¹J_{C,H}=169.0 Hz), 78.6 (C-4_{A}), 76.1 (C-3_{B}), 72.8 (C-5_{A}), 70.8 (OCH₂), 70.2 (OCHₗᵢₙₖₑᵣ), 67.6 (C-3_{A}), 67.3 (C-5_{B}), 62.3 (OCH₂), 58.1 (OCH₃), 54.7 (C-4_{B}), 51.7 (C-2_{A}), 50.8 (C-2_{B}), 22.2 (2C, CH_{3Ac}), 15.5 (C-6_{B}). HRMS (ESI⁺): m/z [M+H]⁺ calcd for C₂₀H₃₆N₃O₁₂, 510.2293; found 510.2292.

(S)-2,3-Dihydroxy-1-propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (54b). The fully protected tetrasaccharide 50b (78 mg, 41 μmol) was subjected to hydrogenation-mediated deprotection (protocol 1). Tetrasaccharide 54b was obtained as a white solid (13 mg, 14 μmol, 35%). Tetrasaccharide 54b had RP-HPLC (215 nm) Rₜ=7.9 min (conditions A). ¹H NMR (D₂O, partial) δ 4.87 (d, 1H, J_{1,2}=8.1 Hz, H-1_{A}), 4.80-4.74 (mₚₒ, 4H, H-5_{A}, H-5_{B1}, H-1_{A}, H-1_{B1}), 4.48-4.45 (m, 2H, H-4_{A}, H-1_{B}), 4.16-4.10 (m, 2H, H-3_{B}, H-3_{B1}), 4.02-3.98 (m, 3H, H-5_{B}, H-5_{B1}), 3.93-3.89 (mₚₒ, 1H, H-2_{A}), 3.86-3.66 (m, 9H), 3.59-3.55 (m, 2H, H₁₋ₗᵢₙₖₑᵣ), 3.49-3.47 (mₚₒ, 1H, H₂₋ₗᵢₙₖₑᵣ), 3.48 (brs, 3H, OCH₃), 1.99, 1.97, 1.94 (2s, 12H, CH_{3Ac}), 1.29 (dₚₒ, 6H, J_{5,6}=6.0 Hz, H-6_{B}, H-6_{B1}). ¹³C NMR (D₂O, partial) δ 174.6, 174.5 (5C, 4NHCO, C-6_{A}), 174.1 (C-6_{A}), 102.8 (C-1_{B1}, ¹J_{C,H}=166.0 Hz), 101.8 (C-1_{B}, ¹J_{C,H}=162.4 Hz), 101.1 (C-1_{A}, ¹J_{C,H}=166.4 Hz), 100.9 (C-1_{A}, ¹J_{C,H}=167.0 Hz), 78.5 (C-4_{A}), 76.5 (C-3_{B1}), 76.0 (3C, C-3_{B}, C-4_{A1}, C-5_{A1}), 72.7 (C-5_{A}), 70.8 (OCH₂), 70.2 (OCHₗᵢₙₖₑᵣ), 67.6 (C-3_{A}), 67.3 (2C, C-5_{B}, C-5_{B1}), 62.3, 58.2 (OCH₂), 58.2 (OCH₃), 54.8, 54.7 (2C, C-4_{B1}, C-4_{B}), 51.7, 51.5 (2C, C-2_{A}, C-2_{A1}), 51.0, 50.9 (2C, C-2_{B}, C-2_{B1}), 22.4, 22.2 (2C, CH_{3Ac}), 15.6, 15.5 (2C, C-6_{B}, C-6_{B1}). HRMS (ESI⁺): m/z [M+H]⁺ calcd for C₃₆H₆₁N₆O₂₁ 913.3884; found 913.3864. HRMS (ESI⁺): m/z [M+Na]⁺ calcd for C₃₆H₆₀N₆O₂₁Na, 935.3704; found 935.3680. HRMS (ESI⁺): m/z [M+2H]2⁺ calcd for C₃₆H₆₁N₆O₂₁ 457.1979; found 457.1972.

(S)-2,3-Dihydroxy-1-propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (55b) The fully protected hexasaccharide 51b (75 mg, 28 μmol) was subjected to hydrogenation-mediated deprotection (protocol 1). Hexasaccharide 55b was obtained as a white solid (8.5 mg, 9.3 μmol, 24%). ¹H NMR (D₂O, partial) δ 4.90 (d, 1H, J_{1,2}=8.4 Hz, H-1_{A}), 4.81-4.70 (mₚₒ, 5H, H-5_{A}, 2H-1_{A}, 2H-1_{B}), 4.70-4.65 (mₚₒ, 2H, 2H-5_{A}), 4.49-4.48 (m, 3H, 2H-4_{A}, H-1_{B}), 4.17-4.11 (m, 3H, H-3_{B}), 4.02-3.93 (m, 4H, H-4_{A}, 3H-5_{B}), 3.94-3.67 (m, 14H), 3.60-3.55 (m, 2H, H₁₋ₗᵢₙₖₑᵣ), 3.49 (brs, 3H, OCH₃), 3.47-3.45 (m, 1H, H₁₋ₗᵢₙₖₑᵣ), 3.01-1.95 (mₚₒ, 18H, CH_{3Ac}), 1.30 (dₚₒ, 9H, J_{5,6}=6.0 Hz, H-6_{B}), ¹³C NMR (D₂O, partial) δ 174.6, 174.5, 174.4, 174.1 (5C, 4NHCO_{A,B}, C-6_{A2}), 172.9, 172.4 (2C, C-6_{A}, C-6_{A1}), 102.9 (2C, C-1_{B1}, C-1_{B2}, ¹J_{C,H}=166.0 Hz), 101.8 (C-1_{B}, ¹J_{C,H}=163.0 Hz), 101.1 (2C, C-1_{A}, C-1_{A1}, ¹J_{C,H}=167.0 Hz), 100.9 (C-1_{A2}, ¹J_{C,H}=169.0 Hz), 78.6, 76.7 (C-4_{A}, C-4_{A1}), 76.3, 76.0 (C-5_{A}, C-5_{A1}), 75.9, 75.7 (C-3_{B}), 72.9, 70.8 (2C, OCH₂), 70.2 (OCHₗᵢₙₖₑᵣ), 67.6 (3C, C-3_{A}, C-3_{A1}, C-3_{A2}), 67.3 (3C, C-5_{B}, C-5_{B1}, C-5_{B2}), 62.3 (OCH₂), 58.1 (OCH₃), 54.8 (3C, C-4_{B1}, C-4_{B}, C-4_{B2}), 51.7 (3C, C-2_{A}, C-2_{A1}, C-2_{A2}), 51.0, 50.9 (3C, C-2_{B}, C-2_{B1}, C-2_{B2}), 22.3, 22.2 (6C, CH_{3Ac}), 15.6, 15.5 (3C, C-6_{B}, C-6_{B1}, C-6_{B2}). HRMS (ESI⁺): m/z [M+2H]²⁺ calcd for C₅₂H₈₇N₉O₃₀ 658.7774; found 658.7754.

### Example 9: Linker-Equipped Oligosaccharides Featuring a 4_{A}-Endchain Hydroxyl Group

Azidopropyl Aglycon as Linker Precursor

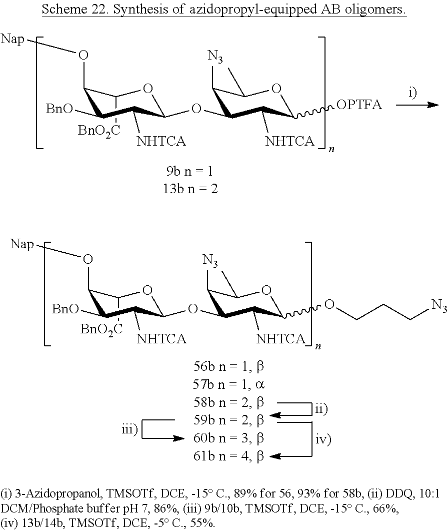

Scheme 22. Synthesis of azidopropyl-equipped AB oligomers. (i) 3-Azidopropanol, TMSOTf, DCE, −15° C., 89% for 56, 93% for 58b, (ii) DDQ, 10:1 DCM/Phosphate buffer pH 7, 86%, (iii) 9b/10b, TMSOTf, DCE, −15° C., 66%, (iv) 13b/14b, TMSOTf, DCE, −5° C., 55%.

3-Azidopropyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranoside (56b) and 2-Azidopropyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-α-D-galactopyranoside (57b). A solution of donors 9b/10b (1.0 g, 876 μmol, 1.0 equiv) and 3-azidopropanol (161 μL, 1.75 mmol, 2.0 equiv) in anhyd. DCE (14 mL) was stirred with freshly activated 4 Å MS (1.0 g) for 30 min at rt under an Ar atmosphere. The reaction mixture was cooled to −15° C. and TMSOTf (11 μL, 61 μmol, 0.07 equiv.) was added. After 40 min at −15° C., the mixture was quenched with Et₃N (12 μL, 0.1 equiv.), filtered and concentrated. Flash chromatography (Tol/EtOAc 4:1) gave by order of elution the α-isomer 57b (40 mg, 38 μmol, 4%) as a white solid and the desired disaccharide 56b (830 mg, 787 μmol, 89%) as a white solid. Disaccharide 56b had R_{f} 0.6 (Tol/EtOAc 4:1). ¹H NMR (CDCl₃) δ 7.85-7.74 (m, 3H, H_{Ar}), 7.70 (brs, 1H, H_{Ar}), 7.52-7.48 (m, 2H, H_{Ar}), 7.42-7.27 (m, 11H, H_{Ar}), 6.80 (d, 1H, J_{2,NH}=7.4 Hz, NH_{B}), 6.72 (d, 1H, J_{2,NH}=7.9 Hz, NH_{A}), 5.26-5.19 (ddₚₒ, 3H, H-1_{A}, CH_{2Bn-6}), 4.85 (d, 1H, J_{4,5}=4.6 Hz, H-5_{A}), 4.74 (brs, 2H, CH_{2Nap}), 4.71 (dₚₒ, 1H, J_{1,2}=8.4 Hz, H-1_{B}), 4.53 (dₚₒ, 1H, J=12.0 Hz, CH_{2Bn}), 4.50 (ddₚₒ, 1H, J_{3,4}=4.0 Hz, J_{2,3}=10.2 Hz, H-3_{B}), 4.45 (dₚₒ, 1H, CH_{2Bn}), 4.24-4.19 (m, 1H, H-2_{A}), 4.10 (dd, 1H, J_{3,4}=2.8 Hz, H-4_{A}), 3.95-3.89 (m, 1H, OCH₂), 3.86 (brd, 1H, H-4_{B}), 3.84 (dd, 1H, J_{2,3}=8.1 Hz, H-3_{A}), 3.67-3.51 (mₚₒ, H-2_{B}, H-5_{B}, OCH₂), 3.37 (t, 2H, J=6.6 Hz, CH₂N₃), 1.89-1.77 (m, 2H, CH₂), 1.28 (d, 3H, J_{5,6}=6.0 Hz, H-6_{B}). ¹³C NMR (CDCl₃) δ 168.9 (C-6_{A}), 162.0, 161.7 (CO_{NHTCA}), 137.4, 134.9, 134.5, 133.1 (2C) (C_{q,Ar}), 128.7, 128.6, 128.4, 128.3, 128.2, 128.0, 127.9, 127.7, 127.0, 126.2, 126.1, 125.9 (C_{Ar}), 99.1 (C-1_{B}, ¹J_{C,H}=163 Hz), 98.8 (C-1_{A}, ¹J_{C,H}=170 Hz), 92.4, 92.1 (2C, CCl₃), 76.1 (C-3_{B}), 73.4 (C-3_{A}), 72.0 (C-4_{A}, CH_{2Nap}), 71.9 (CH_{2Bn}), 71.8 (C-5_{A}), 69.4 (C-5_{B}), 67.5 (CH_{2Bn-6}), 66.3 (OCH₂), 65.1 (C-4_{B}), 55.1 (C-2_{B}), 53.1 (C-2_{A}), 48.1 (CH₂N₃), 29.0, 17.3 (C-6_{B}). HRMS (ESI⁺): m/z [M+NH₄]⁺ calcd for C₄₄H₄₈Cl₆N₉O₁₀, 1072.1650, found 1072.1645.

The side-product 57b had R_{f} 0.65 (Tol/EtOAc 4:1). ¹H NMR (CDCl₃) δ 7.86-7.75 (m, 3H, H_{Ar}), 7.71 (brs, 1H, H_{Ar}), 7.53-7.48 (m, 2H, H_{Ar}), 7.45-7.16 (m, 11H, H_{Ar}), 7.05 (d, 1H, J_{2,NH}=7.4 Hz, NH_{B}), 6.81 (d, 1H, J_{2,NH}=7.2 Hz, NH_{A}), 5.41 (d, 1H, J_{1,2}=6.2 Hz, H-1_{A}), 5.19 (ddₚₒ, 2H, J=12.2 Hz, CH_{2Bn-6}), 4.90 (d, 1H, J_{1,2}=3.8 Hz, H-1_{B}), 4.81 (d, 1H, J_{4,5}=3.8 Hz, H-5_{A}), 4.76 (brs, 2H, CH_{2Nap}), 4.46-4.38 (mₚₒ, 3H, H-2_{B}, CH_{2Bn}), 4.24-4.15 (mₚₒ, 3H, H-2_{A}, H-4_{A}, H-3_{B}), 3.94 (ddₚₒ, 1H, H-4_{B}), 3.90-3.85 (mₚₒ, 2H, H-3_{A}, H-5_{B}), 3.76-3.70 (m, 1H, OCH₂), 3.50-3.44 (m, 1H, OCH₂), 3.35 (t, 2H, J=6.4 Hz, CH₂N₃), 1.85-1.78 (m, 2H, CH₂), 1.24 (d, 3H, J_{5,6}=6.2 Hz, H-6_{B}). ¹³C NMR (CDCl₃) δ 168.9 (C-6_{A}), 162.0, 161.7 (CO_{NHTCA}), 136.9, 134.8, 134.5, 133.1 (2C) (C_{q,Ar}), 129.0, 128.9, 128.8, 128.7, 128.6, 128.5, 128.4, 128.3, 128.2 (2C), 128.0, 127.9, 127.7, 127.1, 126.2, 126.1, 126.0, 125.2 (C_{Ar}), 97.9 (C-1_{A}, ¹J_{C,H}=169 Hz), 96.8 (C-1_{B}, ¹J_{C,H}=175 Hz), 92.5, 92.1 (2C, CCl₃), 75.4 (C-3_{B}), 73.1 (C-3_{A}), 72.7 (C-5_{A}), 72.0 (CH_{2Nap}), 71.9 (C-4_{A}), 71.6 (CH_{2Bn}), 67.5 (CH_{2Bn-6}), 65.6 (C-5_{B}), 65.1 (OCH₂), 64.9 (C-4_{B}), 53.6 (C-2_{A}), 50.8 (C-2_{B}), 48.2 (CH₂N₃), 28.7 (CH_{2,linker}), 17.2 (C-6_{B}). HRMS (ESI⁺): m/z [M+NH₄]⁺ calcd for C₄₄H₄₈Cl₆N₉O₁₀, 1072.1650, found 1072.1646.

3-Azidopropyl (benzyl 3-O-benzyl-2-deoxy-4-O-(2-naphthylmethyl)-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (58b). A solution of donor 13b/14b (3.8 g, 1.94 mmol, 1.0 equiv) and 3-azidopropanol (358 μL, 3.88 mmol, 2.0 equiv) in anhyd DCE (50 mL) was stirred with freshly activated 4 Å MS (3.0 g) for 45 min at rt under an Ar atmosphere. The reaction mixture was cooled to −15° C. and TMSOTf (25 μL, 136 μmol, 0.07 equiv.) was added. After stirring for 1 h at −15° C., Et₃N (20 μL, 0.1 equiv.) was added. The suspension was passed through a pad of Celite, solids were washed with DCM, and volatiles were evaporated. Flash chromatography (Tol/ACN 90:10) of the residue gave the desired tetrasaccharide 58b (3.4 g, 1.82 mmol, 93%) as a white solid. Tetrasaccharide 58b had R_{f} 0.55 (Tol/ACN 4:1). ¹H NMR (CDCl₃) δ 7.84-7.74 (m, 3H, H_{Ar}), 7.69 (brs, 1H, H_{Ar}), 7.51-7.20 (m, 18H, H_{Ar}), 7.08 (d, 1H, J_{2,NH}=6.8 Hz, NH_{B1}), 6.82 (d, 1H, J_{2,NH}=7.2 Hz, NH_{B}), 6.67 (d, 1H, J_{2,NH}=8.0 Hz, NH_{A}), 6.54 (d, 1H, J_{2,NH}=7.6 Hz, NH_{A1}), 5.30-5.18 (ddₚₒ, 4H, CH_{2Bn-6}), 5.17 (dₚₒ, 1H, J_{1,2}=8.0 Hz, H-1_{A}), 5.07 (d, 1H, J_{1,2}=5.0 Hz, H-1_{A1}), 4.83 (dₚₒ, 1H, J_{4,5}=5.2 Hz, H-5_{A1}), 4.81 (dₚₒ, 1H, J_{1,2}=8.0 Hz, H-1_{B1}), 4.77-4.67 (mₚₒ, 4H, CH₂Nap, H-5_{A}, H-1_{B}), 4.65 (ddₚₒ, 1H, J_{3,4}=3.6 Hz, J_{2,3}=10.6 Hz, H-3_{B1}), 4.59-4.55 (m, 2H, H-3_{B}, CH_{2Bn}), 4.55 (dₚₒ, 1H, J=12.6 Hz, CH_{2Bn}), 4.52 (brs, 2H, CH_{2Bn}), 4.31-4.25 (m, 2H, H-4_{A}, H-2_{B1}), 4.08 (dd, 1H, J_{3,4}=3.0 Hz, H-4_{A1}), 3.94-3.88 (mₚₒ, 3H, H-2_{A}, H-4_{B}, OCH₂), 3.84-3.73 (mₚₒ, H-3_{A}, H-3_{A1}, H-4_{A1}), 3.68-3.60 (m, 2H, H-2_{B}, H-5_{B}), 3.56-3.51 (m, 2H, H-2_{B1}, OCH₂) 3.49-3.43 (m, 1H, H-5_{B1}), 3.36 (t, 2H, J=6.6 Hz, CH₂N₃), 1.86-1.76 (m, 2H, CH₂), 1.31 (d, 3H, J_{5,6}=6.0 Hz, H-6_{B}), 1.31 (d, 3H, J_{5,6}=6.0 Hz, H-6_{B1}). ¹³C NMR (CDCl₃) δ 168.9, 168.2 (2C, C-6_{A}, C-6_{A1}), 162.1, 161.9, 161.7, 161.6 (4C, CO_{NHTCA}), 137.3, 137.0, 135.0, 134.9, 134.6, 133.1 (2C) (C_{q,Ar}), 129.1, 129.0, 128.8 (2C), 128.7 (2C), 128.5, 128.4 (2C), 128.3, 128.2, 128.0, 127.9, 126.9, 126.2, 126.1, 125.8 (C_{Ar}), 99.2 (C-1_{B}, ¹J_{C,H}=162 Hz, C-1_{B1}, ¹J_{C,H}=166 Hz), 97.9 (C-1_{A}, ¹J_{C,H}=170.0 Hz), 92.4, 92.3, 92.1 (4C, CCl₃), 75.2 (2C, C-5_{A}, C-3_{B1}) 74.8 (C-3_{B}), 73.3, 73.1 (C-3_{A}, C-3_{A1}), 72.5 (CH_{2Nap}), 72.0 (C-4_{A}), 71.9 (CH_{2Bn}), 71.8 (CH_{2Bn}), 71.6 (C-4_{A1}), 69.4 (C-5_{B}), 68.9 (C-5_{B1}), 67.6 (CH_{2Bn-6}), 67.4 (CH_{2Bn-6}), 66.2 (OCH₂), 65.4, 65.1 (2C, C-4_{B}, C-4_{B1}), 55.4, 55.2 (2C, C-2_{B}, C-2_{B1}), 53.9, 52.7 (2C, C-2_{A}, C-2_{A1}), 48.1 (CH₂N₃), 29.0 (CH_{2,linker}), 17.3, 17.2 (2C, C-6_{B}, C-6_{B1}). HRMS (ESI⁺): m/z [M+NH₄]⁺ calcd for C₇₄H₇₇Cl₁₂N₁₄O₁₉ 1885.1746, found 1885.1760.

3-Azidopropyl (benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (59b). Tetrasaccharide 58b (2.5 g, 1.33 mmol, 1.0 equiv.) was dissolved in DCM (25 mL) and phosphate buffer pH 7 (3 mL) was added. The biphasic mixture was cooled to 0° C. and DDQ (608 mg, 2.67 mmol, 2.0 equiv.) was added, and the biphasic mixture was stirred while keeping the bath temperature between 0-10° C. At completion, 5% aq. NaHCO₃ (50 mL) was added and the biphasic mixture was diluted with DCM (50 mL). The DCM layer was separated, washed with brine, dried over Na₂SO₄, filtered, and concentrated under reduced pressure. The residue was purified by flash chromatography (Tol/ACN 85:15→86:14) to give alcohol 59b (2.0 g, 1.15 mmol, 86%) as a white solid. Tetrasaccharide 59b had R_{f} 0.35 (Tol/ACN 4:1). ¹H NMR (CDCl₃) δ 7.84-7.74 (m, 20H, H_{Ar}), 6.98 (d, 1H, J_{2,NH}=6.8 Hz, NH_{B1}), 6.79 (d, 1H, J_{2,NH}=7.2 Hz, NH_{B}), 6.75 (d, 1H, J_{2,NH}=8.0 Hz, NH_{A}), 6.52 (d, 1H, J_{2,NH}=6.8 Hz, NH_{A1}), 5.31-5.20 (ddₚₒ, 4H, CH_{2Bn-6}), 5.13 (d, 1H, J_{1,2}=7.6 Hz, H-1_{A}), 4.90 (d, 1H, J_{1,2}=4.0 Hz, H-1_{A1}), 4.82 (dₚₒ, 1H, J_{4,5}=8.4 Hz, H-1_{B}), 4.72-4.69 (mₚₒ, 5H, CH_{2Bn}, H-5_{A}, H-5_{A1}, H-1_{B1}), 4.46-4.57 (mₚₒ, 2H, H-3_{B1}, H-3_{B}), 4.49-4.41 (m, 2H, CH_{2Bn}), 4.34-4.30 (m, 1H, H-2_{A}), 4.27 (t, 1H, J_{4,5}=J_{3,4}=2.4 Hz, H-4_{A}), 4.15 (mₚₒ, 1H, H-4_{A1}), 3.95-3.88 (mₚₒ, 3H, H-2_{A}, H-4_{B}, OCH₂), 3.85 (d, 1H, J_{3,4}=2.4 Hz, H-4_{A1}), 3.82-3.78 (ddₚₒ, 1H, H-3_{A1}), 3.73 (d, 1H, J_{3,4}=3.0 Hz, H-4_{A}), 3.65-3.42 (mₚₒ, 5H, H-2_{B}, H-5_{B}, H-2_{B1}, OCH₂, H-5_{B1}), 3.35 (t, 2H, J=6.6 Hz, CH₂N₃), 2.83 (brs, 1H, OH), 1.88-1.76 (m, 2H, CH₂), 1.30 (d, 3H, J_{5,6}=6.0 Hz, H-6_{B}), 1.17 (d, 3H, J_{5,6}=6.2 Hz, H-6_{B1}). ¹³C NMR (CDCl₃) δ 168.8, 168.2 (CO_{COOBn}), 162.2, 161.9, 161.7, 161.6 (CO_{NHTCA}), 137.8, 137.0, 136.9, 135.0, 134.9 (2C) (C_{q,Ar}), 129.1, 129.0, 128.8, 128.7 (2C), 128.6, 128.5, 128.4 (2C), 128.2, 125.2 (C_{Ar}), 99.5 (C-1_{A1}, ¹J_{C,H}=168 Hz), 99.0 (C-1_{B}, ¹J_{C,H}=162 Hz), 99.0 (C-1_{B1}, ¹J_{C,H}=165 Hz), 97.9 (C-1_{A}, ¹J_{C,H}=170 Hz), 92.4, 92.3, 92.0 (4C, CCl₃), 75.8 (C-3_{B}), 75.0 (C-5_{A}), 74.7 (C-3_{A}, C-3_{B1}), 72.9 (C-4_{A1}), 72.4, 72.3 (CH_{2Bn}), 72.1 (C-3_{A1}), 71.4 (C-4_{A1}), 69.5 (C-5_{B1}), 68.8 (C-5_{B}), 67.6 (CH_{2Bn-6}), 67.5 (CH_{2Bn-6}), 66.3 (OCH₂), 65.4, 65.1 (2C, C-4_{B}, C-4_{B1}), 55.4 (2C, C-2_{B}, C-2_{B1}), 53.9, 51.3 (2C, C-2_{A}, C-2_{A1}), 48.1 (CH₂N₃), 29.0 (CH_{2,linker}), 17.3, 17.1 (2C, C-6_{B}, C-6_{B1}). HRMS (ESI⁺): m/z [M+NH₄]⁺ calcd for C₆₃H₆₉Cl₁₂N₁₄O₁₉ 1745.1120, found 1745.1117.

3-Azidopropyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (60b). A solution of disaccharide donor 9b/10b (694 mg, 608 μmol, 1.05 equiv.) and azidopropyl tetrasaccharide 59b (1.0 g, 579 μmol, 1.0 equiv.) in anhyd. DCE (15 mL) was stirred with freshly activated 4 Å MS (1.2 g) for 1 h at rt under an Ar atmosphere. The reaction mixture was cooled to −15° C. and TMSOTf (8 μL, 43 μmol, 0.07 equiv.) was added. After 40 min at −15° C., Et₃N (12 μL, 0.1 equiv.) was added. After stirring for 10 min, the suspension was filtered by passing through a pad of Celite, solids were washed with DCM, and the filtrate was concentrated under vacuum. Flash chromatography (Tol/ACN 84:14→85:15) gave hexasaccharide 60b (850 mg, 317 μmol, 55%) as a white solid. The azidopropyl glycoside 60b had R_{f} 0.3 (Tol/ACN 4:1). HRMS (ESI⁺): m/z [M+NH₄]⁺ calcd for C₁₀₄H₁₀₆Cl₁₈N₁₉O₂₈ 2700.1824, found 2700.1892.

3-Azidopropyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (61b). A solution of tetrasaccharide donors 13b/14b (1.07 g, 547 μmol, 1.05 equiv.) and azidopropyl tetrasaccharide 59b (900 mg, 521 μmol, 1.0 equiv) in anhyd. DCE (22 mL) was stirred with freshly activated 4 Å MS (1.5 g) for 1 h at rt under an Ar atmosphere. The reaction mixture was cooled to −5° C. and TMSOTf (7 μL, 38 μmol, 0.07 equiv.) was added. After stirring for 40 min at −5° C., Et₃N (8 μL, 0.1 equiv.) was added. After stirring for 10 min, solids were filtered and washed with DCM. The filtrate was concentrated under vacuum. Flash chromatography (Tol/ACN 84:14→84:16) of the residue gave azidopropyl octasaccharide 61b (850 mg, 317 μmol, 55%) as a white solid. The coupling product 61b had R_{f} 0.25 (Tol/ACN 4:1). HRMS (ESI⁺): m/z [M+2NH₄]²⁺ calcd for C₁₃₄H₁₃₉Cl₂₄N₂₅O₃₇ 1770.6082, found 1770.6075.

Additional Routes to Linker-Equipped Glycosides
Cbz-masked aminopropyl linker
Acetal-masked ketone-encompassing linker

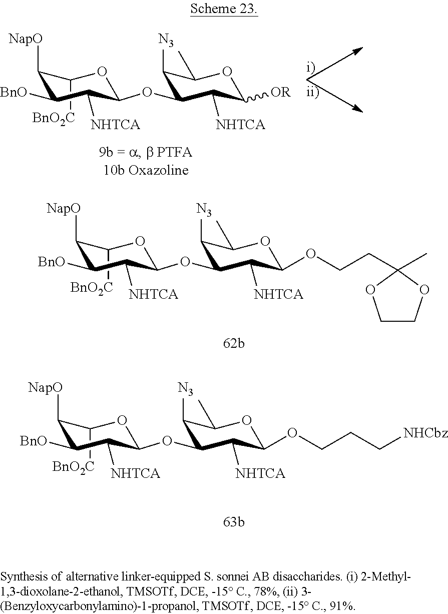

2-Methyl-1,3-dioxolane-2-ethyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranoside (62b). A solution of donor 9b/10b (550 mg, 482 μmol, 1.0 equiv) and 2-methyl-1,3-dioxolane-2-ethanol (127 mg, 963 μmol, 2.0 equiv) in anhyd. DCE (8 mL) was stirred with freshly activated 4 Å MS (800 mg) for 30 min at rt under an Ar atmosphere. The reaction mixture was cooled to −15° C. and TMSOTf (6.0 μL, 34 μmol, 0.07 equiv.) was added. After stirring for 1 h at −15° C., the mixture was quenched with Et₃N (6 μL, 0.1 equiv.), filtered, and concentrated. Flash chromatography using Tol/EtOAc (80:20) gave the desired disaccharide 62b (410 mg, 405 μmol, 78%) as a white solid. Disaccharide 62b had R_{f} 0.4 (Tol/EtOAc 4:1) and HRMS (ESI⁺): m/z [M+NH₄]⁺ calcd for C₄₇H₅₃Cl₆N₆O₁₂, 1103.1821; found 1103.1847.

3-(Benzyloxycarbonylamino)-1-propyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranoside (63b). A solution of donor 9b/10b (600 mg, 525 μmol, 1.0 equiv.) and 3-(benzyloxycarbonylamino)-1-propanol (220 mg, 1.05 mmol, 2.0 equiv.) in anhyd. DCE (9 mL) was stirred with freshly activated 4 Å MS (800 mg) for 30 min at rt under an Ar atmosphere. The reaction mixture was cooled to −15° C. and TMSOTf (7.0 μL, 61 μmol, 0.07 equiv.) was added. After stirring for 40 min at −15° C., Et₃N (7 μL, 0.1 equiv.) was added to the reaction mixture, which was stirred at rt for 10 min. The suspension was filtered and concentrated. Flash chromatography (Tol/EtOAc 7:3) gave the desired disaccharide 63b (560 mg, 481 μmol, 91%) as a white solid. Disaccharide 63b had R_{f} 0.35 (Tol/EtOAc 7:3). HRMS (ESI⁺): m/z [M+NH₄]⁺ calcd for C₅₂H₅₆Cl₆N₇O₁₂, 1180.2113; found 1180.2091.

### Example 10. Linker-Equipped Oligosaccharides Featuring a B-Endchain Residue

Azidopropyl Aglycon as Linker Precursor

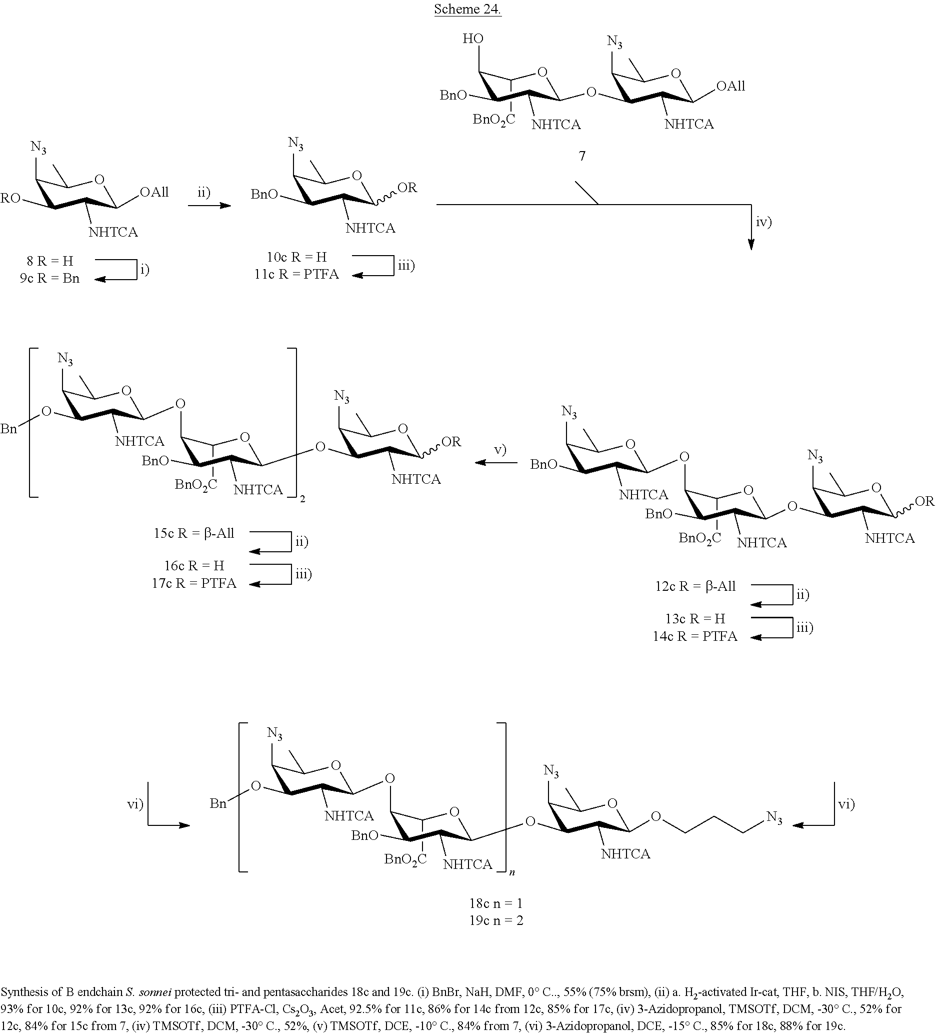

Allyl 4-azido-3-O-benzyl-2-trichloroacetamido-2,4,6-trideoxy-β-D-galactopyranoside (9c). A solution of alcohol 8 (6.0 g, 16.1 mmol, 1.0 equiv) in anhyd. DMF (40 mL) was cooled to 0° C. under an Ar atmosphere. Benzyl bromide (2.3 mL, 19.3 mmol, 1.2 equiv.) was added followed by the portionwise addition of NaH (60% in mineral oil, 1.16 g, 48.3 μmol, 3.0 equiv.). After stirring for 24 h at 0° C., diluted aq. NH₄Cl (200 mL) was added and the aq. layer was washed with DCM (200 mL) twice. The combined organic parts were dried over Na₂SO₄, filtered and the filtrate was concentrated under reduced pressure. Flash chromatography of crude residue (Tol/EtOAc 90:10-84:16) gave the desired benzyl ether 9c (4.1 mg, 8.87 mmol, 55%) as a white solid. Further elution (Tol/EtOAc 90:10-60:40) gave the unreacted starting material 8 (1.6 g, 26%) corresponding to a 75% corrected yield. The benzyl ether 9c had R_{f} 0.6 (Tol/EtOAc 7:3). ¹H NMR (CDCl₃) δ 7.36-7.28 (m, 5H, H_{Ar}), 7.08 (d, 1H, J_{NH,2}=7.0 Hz, NH), 5.91-5.81 (m, 1H, CH_{All}), 5.28-5.24 (mₚₒ, 1H, CH_{2All}), 5.19-5.16 (mₚₒ, 1H, CH_{2All}), 4.93 (d, 1H, J_{1,2}=8.6 Hz, H-1), 4.70 (d, 1H, J=10.8 Hz, CH_{2Bn}), 4.62 (d, 1H, CH_{2Bn}), 4.50 (dd, 1H, J_{2,3}=10.6 Hz, J_{3,4}=3.6 Hz, H-3), 4.35-4.30 (mₚₒ, 1H, CH_{2All}), 4.09-4.04 (mₚₒ, 1H, CH_{2All}), 3.75 (brd, 1H, H-4), 3.69-3.59 (mₚₒ, 2H, H-2, H-5), 1.35 (d, 3H, J_{5,6}=6.0 Hz, H-6). ¹³C NMR (CDCl₃) δ 162.0 (CO_{NHTCA}), 137.0 (C_{q,Ar}), 133.6 (CH_{A}n), 128.6, 128.3 (C_{Ar}), 117.9 (CH_{2All}), 97.7 (C-1, ¹J_{C,H}=163 Hz), 92.4 (CCl₃), 76.1 (C-3), 72.6 (CH_{2Bn}), 70.2 (CH_{2All}), 69.0 (C-5), 63.0 (C-4), 56.0 (C-2), 17.6 (C-6). HRMS (ESI⁺): m/z [M+NH₄]⁺ calcd for C18H₂₅Cl₃N₅O₄ 480.0967; found 480.0967.

4-Azido-3-O-benzyl-2-trichloroacetamido-2,4,6-trideoxy-α/β-D-galactopyranose (10c). [Ir(COD)(PMePh₂)₂]PF₆ (200 mg, 237 μmol, 0.03 equiv.) was dissolved in anhyd. THF (10 mL) and stirred for 30 min under an H₂ atmosphere. The resulting yellow solution was degassed repeatedly with Ar and transferred by means of a cannula into a solution of allyl glycoside 9c (3.65 g, 7.89 mmol, 1.0 equiv.) in anhyd. THF (70 mL). After 2 h stirring at rt, a TLC analysis (Tol/EtOAc 85:15) showed the complete disappearance of the starting material and presence of a slightly less polar spot. NIS (2.1 g, 9.47 mmol, 1.2 equiv.) in THF/H₂O (1:1, 20 mL) was added. After stirring for 1 h at rt, a TLC analysis (Tol/EtOAc 7:3) revealed the full consumption of the isomerized intermediate and the presence of a more polar spot. 10% Aq. Na₂SO₃ was added and volatiles were evaporated. The aq. phase was extracted with DCM (100 mL) three times. The combined organic layers were washed with water and brine, dried over Na₂SO₄, filtered, and concentrated under vacuum. Purification of the residue by flash chromatography (Tol/EtOAc 80:20→70:30) yielded the expected α/β-hemiacetal 10c (3.1 g, 7.34 mmol, 93%) as a white floppy solid. Hemiacetal 10c had R_{f} 0.5, 0.2 (Tol/EtOAc 7:3). HRMS (ESI⁺): m/z [M+NH₄]⁺ calcd for C₁₅H₂₁Cl₃N₅O₄ 440.0654; found 440.0652.

4-Azido-3-O-benzyl-2,4,6-trideoxy-2-trichloroacetamido-α/β-D-galactopyranosyl (N-phenyl)trifluoroacetimidate (11c). Hemiacetal 10c (3.0 g, 7.10 mmol, 1.0 equiv.) was dissolved in acetone (35 mL). PTFA-Cl (1.46 mL, 9.24 mmol, 1.3 equiv.) was added followed by Cs₂CO₃ (2.7 g, 8.53 mmol, 1.2 equiv.). After stirring for 2 h at rt, the reaction mixture was filtered, washed with acetone and the filtrate was concentrated under reduced pressure. The crude donor was purified by passing through a short silica column (cHex/EtOAc 90:10) to give the PTFA donor 11c as a mixture of α/β-isomers (3.9 g, 6.57 mmol, 92.5%). The α-isomer had R_{f} 0.7 (Tol/EtOAc 9:1). ¹H NMR (CDCl₃) δ 7.44-7.35 (m, 5H, H_{Ar}), 7.32-7.24 (m, 2H, H_{Ar}), 7.14-7.11 (ttₚₒ, 1H, H_{Ar}), 6.79 (d, 2H, J=7.6 Hz, H_{Ar}), 6.46 (d, 1H, J_{2,NH}=7.7 Hz, NH), 6.39 (brs, 1H, H-1), 4.82 (d, 1H, J=12.0 Hz, CH_{2Bn}), 4.62 (dₚₒ, 1H, CH_{2Bn}), 4.59-4.53 (mₚₒ, 1H, H-2), 4.07-4.05 (mₚₒ, 1H, H-5), 3.99-3.96 (mₚₒ, 2H, H-3, H-4), 1.36 (d, 1H, J_{5,6}=6.0 Hz, H-6). ¹³C NMR (CDCl₃) δ 161.8 (CO_{NHTCA}), 142.9, 135.5 (C_{q,Ar}), 129.3, 128.9, 128.8, 128.5, 128.1, 126.3, 124.6, 120.5, 119.2 (C_{Ar}), 119.2 (CF₃), 94.1 (C-1_{A}, ¹J_{C,H}=191 Hz), 92.1 (CCl₃), 75.0 (C-3), 71.6 (CH_{2Bn}), 67.7 (C-5), 61.9 (C-4), 50.2 (C-2), 17.4 (C-6). HRMS (ESI⁺): m/z [M+Na]⁺ calcd for C₂₃H₂₁Cl₃F₃N₅O₄Na, 616.0503; found 616.0496.

Allyl 4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (12c). The PTFA donor 11c (3.4 g, 5.73 mmol, 1.0 equiv.) and acceptor 7 (4.99 g, 5.73 mmol, 1.0 equiv.) were mixed and dried over vacuum. The dried mass dissolved in anhyd. DCM (100 mL) was stirred with freshly activated 4 Å MS (6.0 g) for 30 min at rt under an Ar atmosphere. The reaction mixture was cooled to −30° C. and TMSOTf (51 μL, 287 μmol, 0.05 equiv.) was added. After 40 min at −30° C., a TLC analysis (Tol/EtOAc 4:1) showed the absence of PTFA donor and the presence of a new spot. Et₃N was added and after 15 min, the suspension was filtered and volatiles were evaporated. Flash chromatography (Tol/ACN 90:10→88:12) yielded the desired trisaccharide 12c (3.8 g, 3.1 mmol, 52%) as a white solid. Along with the undesired α-isomer (2.6 g, 3.64 mmol, 35%). The desired product 12c had R_{f} 0.4 (Tol/ACN 4:1). ¹H NMR (CDCl₃) δ 7.43-7.15 (m, 15H, H_{Ar}), 7.05 (d, 1H, J_{2,NH}=7.1 Hz, NH_{B1}), 6.76 (d, 1H, J_{2,NH}=7.2 Hz, NH_{B}), 6.51 (d, 1H, J_{2,NH}=6.8 Hz, NH_{A}), 5.89-5.80 (m, 1H, CH_{All}), 5.31-5.18 (mₚₒ, 4H, CH_{2All}, CH_{2Bn-6}), 5.17 (dₚₒ, 1H, J_{1,2}=7.6 Hz, H-1_{A}), 4.90 (d, 1H, J_{1,2}=8.4 Hz, H-1_{B1}), 4.78 (d, 1H, J_{1,2}=8.4 Hz, H-1_{B}), 4.73 (d, 1H, J_{4,5}=2.7 Hz, H-5_{A}), 4.68-4.62 (mₚₒ, 3H, CH_{2Bn}, H-3_{B}), 4.54 (ddₚₒ, 1H, J_{2,3}=10.6 Hz, J_{4,5}=3.4 Hz, H-3_{B1}), 4.47 (ddₚₒ, 2H, J=12.6 Hz, CH_{2Bn}), 4.33-4.29 (mₚₒ, 2H, H-4_{A}, CH_{2All}), 4.05-4.01 (mₚₒ, 1H, CH_{2All}), 3.95-3.86 (mₚₒ, 3H, H-2_{A}, H-3_{A}, H-4_{B}), 3.66 (d, 1H, J_{3,4}=3.4 Hz, H-4_{B1}), 3.61-3.46 (mₚₒ, 4H, H-2_{B}, H-2_{B1}, H-5_{B}, H-5_{B1}), 1.30 (d, 3H, J_{5,6}=6.2 Hz, H-6_{B1}), 1.27 (d, 3H, J_{5,6}=6.4 Hz, H-6_{B}). ¹³C NMR (CDCl₃) δ 168.2 (C-6_{A}), 162.0, 161.9, 161.6 (CO_{NHTCA}), 137.1, 137.0, 135.0 (C_{q,Ar}), 133.5 (CH_{All}), 129.0 (2C), 128.8, 128.7, 128.6, 128.3 (3C), 125.3 (C_{Ar}), 117.9 (CH_{2All}), 99.0 (C-1_{B1}, ¹J_{C,H}=165 Hz), 98.2 (C-1_{A}, ¹J_{C,H}=169 Hz), 97.8 (C-1_{B}, ¹J_{C,H}=162 Hz), 92.5, 92.3 (3C, CCl₃), 75.5 (C-3_{B1}), 75.2 (C-5_{A}), 74.8 (C-3_{B}), 73.0 (C-3_{A}), 72.7 (CH_{2Bn}), 72.4 (CH_{2Bn}), 71.8 (C-4_{A}), 70.0 (CH_{2All}), 69.0 (C-5_{B}), 68.8 (C-5_{B1}), 67.5 (CH_{2Bn-6}), 65.4 (C-4_{B}), 63.0 (C-4_{B1}), 55.8, 55.6 (2C, C-2_{B}, C-2_{B1}), 54.1 (C-2_{A}), 17.5, 17.3 (2C, C-6_{B}, C-6_{B1}). HRMS (ESI⁺): m/z [M+NH₄]⁺ calcd for C₄₈H₅₄Cl₉N₁₀O₁₃ 1293.1063; found 1293.1068.

4-Azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranose (13c). [Ir(COD)(PMePh₂)₂]⁺PF₆⁻ (123 mg, 145 μmol, 0.05 equiv.) was dissolved in anhyd. THF (8 mL) and stirred for 20 min under an H₂ atmosphere. The resulting yellow solution was degassed with a continuous flow of Ar and poured into a solution of allyl glycoside 12c (3.7 g, 2.90 mmol, 1.0 equiv.) in anhyd. THF (50 mL). After stirring for 2 h at rt, NIS (783 mg, 3.48 mmol, 1.2 equiv.) in THF/H₂O was added. After stirring for another 1 h at rt, 10% aq. sodium sulphite was added until full decoloration. Volatiles were evaporated and the aq. phase was extracted with DCM (50 mL) three times. The organic phases were combined, washed with brine, dried over anhyd. Na₂SO₄ and concentrated. Purification by flash chromatography (Tol/EtOAc 75:25→60:40) yielded the α/β hemiacetal 13c (3.3 g, 2.67 mmol, 92%) as a white solid. The α/β-anomer had R_{f} 0.5, 0.25 (Tol/EtOAc 75:25→60:40). HRMS (ESI⁺): m/z [M+NH₄]⁺ calcd for C₄₅H₅₀Cl₉N₁₀O₁₃ 1257.0713; found 1253.0750.

4-Azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl (N-phenyl)trifluoroacetimidate (14c). PTFACl (550 μL, 3.47 mmol, 1.3 equiv.) and Cs₂CO₃ (1.04 g, 3.20 mmol, 1.2 equiv.) were added to hemiacetal 13c (3.3 g, 2.67 mmol, 1.0 equiv.) dissolved in acetone (27 mL). After stirring for 2 h at rt, the reaction mixture was filtered through a pad of Celite and washed with acetone (5 mL) twice. The filtrate was concentrated under reduced pressure and the crude PTFA donor was purified by passing through a short pack of silica (cHex/EtOAc 75:25→65:35). The α/β-PTFA donor 14c was isolated as a solid (3.25 g, 2.31 mmol, 86% over two steps). The PTFA donor had R_{f} 0.75, 0.8 (Tol/EtOAc 7:3). HRMS (ESI⁺): m/z [M+NH₄]⁺ calcd for C₅₃H₅₄Cl₉F₃N₁₁O₁₃ 1424.1046; found 1424.1029.

Allyl 4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1-4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (15c). The PTFA trisaccharide 14c (1.0 g, 711 μmol, 1.0 equiv.) and disaccharide acceptor 7 (619 mg, 711 mmol, 1.0 equiv.) were stirred with freshly activated 4 Å MS (1.0 g) in anhyd. DCE (17 mL) for 1 h under an Ar atmosphere at rt. After cooling to −10° C., TMSOTf (7.7 μL, 43 μmol, 0.06 equiv.) was added and stirring was continued for 45 min while keeping the temperature of the reaction bath at −10° C. At completion, Et₃N (˜10 μL) was added. The suspension was filtered through a fitted funnel and washed with DCM (6 mL) thrice. Volatiles were evaporated and the residue was purified by flash chromatography (Tol/ACN 90:10→86:14) to give pentasaccharide 15c as a white solid (1.2 g, 574 μmol, 84%). The coupling product 15c had R_{f} 0.55 (Tol/ACN 4:1). HRMS (ESI⁺): m/z [M+NH₄]⁺ calcd for C₇₈H₈₃Cl₁₅Cl₄N₁₅O₂₂ 2114.1070; found 2114.1078. ¹H NMR (CDCl₃) δ 7.41-7.17 (m, 25H, H_{Ar}), 7.11 (d, 1H, J_{2,NH}=6.8 Hz, NH_{B2}), 7.02 (d, 1H, J_{2,NH}=6.7 Hz, NH_{B1}), 6.76 (d, 1H, J_{2,NH}=7.2 Hz, NH_{B}), 6.49 (d, 1H, J_{2,NH}=6.8 Hz, NH_{A1}), 6.44 (d, 1H, J_{2,NH}=6.8 Hz, NH_{A}), 5.89-5.80 (m, 1H, CH_{All}), 5.36 (d, 1H, J=12.0 Hz, CH_{2Bn-6}), 5.28-5.22 (mₚₒ, 3H, CH_{2All}, CH_{2Bn-6}), 5.20-5.15 (mₚₒ, 3H, CH_{2All}, CH_{2Bn-6}, H-1_{A}), 5.02 (d, 1H, J_{1,2}=7.6 Hz, H-1_{A1}), 4.94 (d, 1H, J_{1,2}=8.4 Hz, H-1_{B2}), 4.88 (d, 1H, J_{1,2}=8.0 Hz, H-1_{B1}), 4.81 (ddₚₒ, J_{2,3}=10.6 Hz, J_{3,4}=3.6 Hz, H-3_{B1}), 4.78 (dₚₒ, J_{1,2}=8.4 Hz, H-1_{B}), 4.72 (dₚₒ, 1H, J_{4,5}=2.7 Hz, H-5_{A}), 4.68 (dₚₒ, 1H, J_{4,5}=2.6 Hz, H-5_{A1}), 4.67-4.63 (mₚₒ, 3H, H-3_{B}, CH_{2Bn}), 4.58 (ddₚₒ, 1H, J_{2,3}=10.6 Hz, J_{3,4}=3.4 Hz, H-3_{B2}), 4.51 (d, 1H, J=12.2 Hz, CH_{2Bn}), 4.45-4.40 (mₚₒ, 3H, CH_{2Bn}), 4.34-4.28 (mₚₒ, 2H, CH_{2All}, H-4_{A1}), 4.21 (tₚₒ, 1H, H-4_{A}), 4.06-4.00 (mₚₒ, 1H, CH_{2All}), 3.98-3.94 (mₚₒ, 1H, H-2_{A1}), 3.91 (brd, 1H, J_{3,4}=3.4 Hz, H-4_{B}), 3.87-3.85 (mₚₒ, 3H, H-2_{A}, H-3_{A}, H-4_{B1}), 3.81 (dd, 1H, J_{2,3}=10.5 Hz, J_{3,4}=2.5 Hz, H-3_{A1}), 3.66 (brd, 1H, J_{3,4}=3.0 Hz, H-4_{B2}), 3.62-3.39 (mₚₒ, 6H, H-2_{B1}, H-2_{B2}, H-2_{B}3, H-5_{B1}, H-5_{B2}, H-5_{B3}), 1.30 (d, 1H, J_{5,6}=6.2 Hz, H-6_{B}), 1.27 (d, 1H, J_{5,6}=6.4 Hz, H-6_{B}), 1.22 (d, 1H, J_{5,6}=6.4 Hz, H-6_{B}). ¹³C NMR (CDCl₃) δ 168.2, 168.1 (C-6_{A}), 162.1, 161.9, 161.5 (5C, CO_{NHTCA}), 137.1, 137.0, 135.1, 135.0 (C_{q,Ar}), 133.5 (CH_{All}), 129.1 (2C), 129.0, 128.9, 128.2, (2C) (C_{Ar}), 117.9 (CH_{2All}), 99.0 (C-1_{B2}, ¹J_{C,H}=166 Hz), 98.6 (C-1_{B1}, ¹J_{C,H}=166 Hz), 98.4 (C-1_{A1}, ¹J_{C,H}=16 Hz), 98.0 (C-1_{A}, ¹J_{C,H}=168 Hz), 97.8 (C-1_{B}, ¹J_{C,H}=162 Hz), 92.5, 92.4, 92.3 (5C, CCl₃), 75.4 (C-5_{A}, C-5_{A1}, C-3_{B2}), 74.6 (C-3_{B}), 73.6 (C-3_{B1}), 72.8 (2C, C-3_{A}, C-3_{A1}), 72.4 (2C, CH_{2Bn}), 72.1 (CH_{2Bn}), 71.9 (C-4_{A1}), 71.7 (C-4_{A}), 70.0 (CH_{2All}), 69.3, 68.7 (C-5_{B}, C-5_{B1}, C-5_{B2}), 67.5, 67.5 (2C, CH_{2Bn-6}), 65.5 (C-4_{B}), 65.3 (C-4_{B1}), 63.1 (C-4_{B2}), 55.9, 55.8, 55.6 (C-2_{B}, C-2_{B1}, C-2_{B2}), 54.2, 53.8 (C-2_{A}, C-2_{A1}), 17.5, 17.3, 17.2 (C-6_{B}, C-6_{B1}, C-6_{B2}).

4-Azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranose (16c). [Ir(COD)(PMePh₂)₂]⁺PF₆⁻ (22 mg, 26 μmol, 0.05 equiv.) was dissolved in anhyd. THF (6 mL) and stirred for 40 min under a H₂ atmosphere. The resulting yellow solution was degassed with a continuous flow of Ar for 10 min and poured into a solution of allyl glycoside 15c (1.1 g, 527 μmol, 1.0 equiv.) in anhyd. THF (5.0 mL). After stirring for 2 h at rt, NIS (142 mg, 632 μmol, 1.2 equiv.) was added. After stirring for another 1 h at rt, the reaction was quenched with 10% aq. sodium sulphite. Volatiles were removed under vacuum and the aq. phase was extracted with DCM (3×20 mL). The organic phases were combined, washed with brine, dried over anhyd. Na₂SO₄ and concentrated. Purification by flash chromatography (Tol/EtOAc 70:30→60:40) yielded the expected α/β-hemiacetal 16c (1.0 g, 488 μmol, 92%) as a white solid. The α/β-anomers had R_{f} 0.55, 0.7 (Tol/EtOAc 1:1). HRMS (ESI⁺): m/z [M+NH₄]⁺ calcd for C₇₅H₇₉C₁₅N₁₅O₂₂ 2066.0847; found 2066.0888.

4-Azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl (N-phenyl)trifluoroacetimidate (17c). Hemiacetal 16c (1.0 g, 488 μmol, 1.0 equiv.) was dissolved in acetone (6.0 mL). PTFACl (100 μL, 635 μmol, 1.3 equiv.) and Cs₂CO₃ (191 mg, 586 μmol, 1.2 equiv.) were added. After stirring for 2 h at rt, the reaction mixture was filtered through a pad of Celite and solids were washed with acetone (6 mL) twice. The filtrate was concentrated under reduced pressure and the crude PTFA donor was purified by passing through a short pack of silica gel eluting with cHex/EtOAc (70:30→50:50). Fractions corresponding to the product were combined to give the expected PTFA donor 17c as a mixture of α/β-isomers (920 mg, 415 μmol, 85%). HRMS (ESI⁺): calcd for C₈₃H₈₃Cl₁₅F₃N₁₆O₂₂ 2239.1121; found 2239.11185.

3-Azidopropyl 4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (18c). The PTFA donor 14c (1.1 g, 782 μmol, 1.0 equiv.) and 3-azido-1-propanol (144 μL, 1.56 mmol, 2.0 equiv.) were stirred with freshly activated 4 Å MS (1.0 g) in anhyd. DCE (15.6 mL) for 1 h under an Ar atmosphere at rt. After cooling to −15° C., TMSOTf (10 μL, 55 μmol, 0.07 equiv.) was added and stirring was continued for 45 min while keeping the temperature of the reaction bath at −15° C. At completion, Et₃N (˜12 μL) was added. The suspension was filtered through a fitted funnel and washed with DCM (10 mL) twice. Flash chromatography (Tol/ACN 90:10→88:12) of the residue gave the azidopropyl trisaccharide 18c as a white solid (875 mg, 663 μmol, 85%). The coupling product 18c had R_{f} 0.45 (Tol/ACN 4:1). ¹H NMR (CDCl₃) δ 7.43-7.17 (m, 15H, H_{Ar}), 7.01 (d, 1H, J_{2,NH}=6.8 Hz, NH), 6.74 (d, 1H, J_{2,NH}=7.2 Hz, NH), 6.51 (d, 1H, J_{2,NH}=6.8 Hz, NH), 5.30-5.19 (mₚₒ, 3H, CH_{2Bn-6}, H-1_{A}), 4.86 (d, 1H, J_{1,2}=8.4 Hz, H-11), 4.73 (dₚₒ, 1H, J_{4,5}=2.5 Hz, H-5_{A}), 4.70 (dₚₒ, 1H, J_{1,2}=8.1 Hz, H-1_{B}), 4.64 (ddₚₒ, 2H, J=11.2 Hz, CH_{2Bn}), 4.56 (dd, J_{2,3}=10.8 Hz, J_{3,4}=3.6 Hz, H-3_{B}), 4.50-4.43 (mₚₒ, 3H, H-3_{B1}, CH_{2Bn}), 4.29 (t, 1H, J=2.2 Hz, H-4_{A}), 3.94-3.85 (mₚₒ, 4H, OCH₂, H-2_{A}, H-3_{A}, H-4_{B}), 3.66 (d, 1H, J_{3,4}=2.8 Hz, H-4_{B1}), 3.64-3.50 (mₚₒ, 4H, OCH₂, H-2_{B}, H-2_{B1}, H-5_{B}), 3.47 (dqₚₒ, 1H, H-5_{B1}), 3.35 (t, 2H, J=6.6 Hz, NCH₂), 1.88-1.76 (m, 2H, CH₂), 1.29 (d, 6H, J_{5,6}=6.4 Hz, H-6_{B}, H-6_{B1}). ¹³C NMR (CDCl₃) δ 168.2 (C-6_{A}), 161.9 (3C, CO_{NHTCA}), 137.1, 137.0, 135.0 (C_{q,Ar}), 129.0, 128.8 (2C), 128.7, 128.6, 128.3 (2C), 128.2 (C_{Ar}), 99.1 (2C, C-1_{B1}, C-1_{B}, ¹J_{C,H}=168 Hz, ¹J_{C,H}=162 Hz), 98.0 (C-1_{A}, ¹J_{C,H}=168 Hz), 75.6 (C-3_{B1}), 75.3 (C-5_{A}), 74.9 (C-3_{B}), 73.0 (C-3_{A}), 72.7 (CH_{2Bn}), 72.5 (CH_{2Bn}), 71.9 (C-4_{A}), 69.0, 68.9 (2C, C-5_{B}, C-5_{B1}), 67.6 (OCH₂), 66.3 (CH_{2Bn-6}), 65.3 (C-4_{B}), 62.9 (C-4_{B1}), 55.7, 55.3 (2C, C-2_{B}, C-2_{B1}), 54.1 (C-2_{A}), 48.1 (NCH₂), 29.0 (CH₂), 17.5, 17.3 (2C, C-6_{B}, C-6_{B1}). HRMS (ESI⁺): m/z [M+NH₄]⁺ calcd for C₄₈H₅₅Cl₉N₁₃O₁₃ 1336.1234; found 1336.1229.

3-Azidopropyl 4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (19c). The PTFA donor 17c (920 mg, 415 μmol, 1.0 equiv.) and 3-azido-1-propanol (76 μL, 829 μmol, 2.0 equiv.) were stirred with freshly activated 4 Å MS (600 mg) in anhyd. DCE (10 mL) for 1 h under an Ar atmosphere at rt. After cooling to −15° C., TMSOTf (5.0 μL, 29 μmol, 0.07 equiv.) was added and stirring was continued for 45 min while keeping the temperature of the reaction bath at −15° C. At completion, Et₃N (˜10 μL) was added. The suspension was filtered through a fitted funnel and washed with DCM. Volatiles were evaporated and the residue was purified by flash chromatography (Tol/ACN 90:10→88:12) to give the azidopropyl pentasaccharide 19c as a white solid (785 mg, 359 μmol, 88%). The coupling product 19c had R_{f} 0.4 (Tol/ACN 4:1).

¹H NMR (CDCl₃) δ 7.34-7.15 (m, 25H, H_{Ar}), 7.07 (d, 1H, J_{2,NH}=6.8 Hz, NH_{B2}), 7.01 (d, 1H, J_{2,NH}=7.2 Hz, NH_{B1}), 6.76 (d, 1H, J_{2,NH}=7.2 Hz, NH_{B}), 6.50 (d, 1H, J_{2,NH}=6.8 Hz, NH_{A1}), 6.44 (d, 1H, J_{2,NH}=8.0 Hz, NH_{A}), 5.35 (d, 1H, J=12.0 Hz, CH_{2Bn-6}), 5.28-5.17 (mₚₒ, 4H, H-1_{A}, CH_{2Bn-6}), 5.05 (d, 1H, J_{1,2}=7.6 Hz, H-1_{A1}), 4.91 (d, 1H, J_{1,2}=8.4 Hz, H-1_{B2}), 4.84 (d, 1H, J_{1,2}=8.2 Hz, H-1_{B1}), 4.78 (ddₚₒ, J_{2,3}=10.6 Hz, J_{3,4}=4.0 Hz, H-3_{B1}), 4.72 (dₚₒ, 1H, J_{4,5}=2.6 Hz, H-5_{A1}), 4.70 (dₚₒ, J_{1,2}=8.0 Hz, H-1_{B}), 4.69 (dₚₒ, 1H, J_{4,5}=2.8 Hz, H-5_{A}), 4.65 (ddₚₒ, 2H, CH_{2Bn}), 4.58-4.52 (mₚₒ, 2H, H-3_{B2}, H-3_{B}), 4.49-4.40 (mₚₒ, 4H, CH_{2Bn}), 4.31 (tₚₒ, 1H, J_{4,5}=J_{3,4}=2.6 Hz, H-4_{A1}), 4.21 (tₚₒ, 1H, H-4_{A}), 4.00-3.86 (mₚₒ, 6H, H-2_{A}, H-2_{A1}, H-3_{A}, H-3_{A}, H-4_{B}, H-4_{B1}, OCH₂), 3.66 (brd, 1H, J_{3,4}=2.8 Hz, H-4_{B2}), 3.65-3.51 (mₚₒ, 7H, OCH₂,H-2_{B1}, H-2_{B2}, H-2_{B}3, H-5_{B1}, H-5_{B2}, H-5_{B3}), 3.36 (t, 2H, J=6.6 Hz, NCH₂), 1.88-1.76 (mₚₒ, 2H, CH₂), 1.30 (d, 1H, J_{5,6}=6.2 Hz, H-6_{B}), 1.28 (d, 1H, J_{5,6}=6.4 Hz, H-6_{B}), 1.22 (d, 1H, J_{5,6}=6.4 Hz, H-6_{B}). ¹³C NMR (CDCl₃) δ 168.2, 168.1 (C-6_{A}), 162.1, 161.9, 161.6 (5C, CO_{NHTCA}), 137.1, 137.0, 135.1, 134.9 (C_{q,Ar}), 129.1 (2C), 129.0, 128.9, 128.8, 128.7, 128.6 (2C), 128.5, 128.3 (2C) (C_{Ar}), 99.2 (C-1_{B}, ¹J_{C,H}=161 Hz, C-1_{B2}, ¹J_{C,H}=163 Hz), 98.8 (C-1_{B1}, ¹J_{C,H}=167 Hz), 98.3 (C-1_{A1}, ¹J_{C,H}=168 Hz), 97.8 (C-1_{A}, ¹J_{C,H}=169 Hz), 92.5, 92.4, 92.3 (5C, CCl₃), 75.4 (C-5_{A}, C-5_{A1}), 74.6 (C-3_{B1}), 74.6 (C-3_{B}, C-3_{B2}), 72.9 (C-3_{A}, C-3_{A1}), 72.7 (CH_{2Bn}), 72.5 (CH_{2Bn}), 72.1 (CH_{2Bn}), 71.9 (C-4_{A1}), 71.7 (C-4_{A}), 69.0, 68.8, 68.8 (C-5_{B}, C-5_{B1}, C-5_{B2}), 67.5 (2C, CH_{2Bn-6}), 66.2 (OCH₂), 65.4 (C-4_{B}), 65.3 (C-4_{B1}), 63.0 (C-4_{B2}), 55.8, 55.7, 55.6 (C-2_{B}, C-2_{B1}, C-2_{B2}), 54.1, 53.8 (C-2_{A}, C-2_{A1}), 48.1 (NCH₂), 29.0 (CH₂), 17.5, 17.3, 17.2 (C-6_{B}, C-6_{B1}, C-6_{B2}). HRMS (ESI⁺): m/z [M+NH₄]⁺ calcd for C₇₈H₈₄³⁵Cl₁₁³⁷Cl₄N₁₈O₂₂ 2157.1242 found 2157.1284.

### Example 11. Full Deprotection of the 4_{A}-Terminal Oligosaccharides to Provide the Corresponding Aminopropyl-Linker Equipped Oligosaccharides

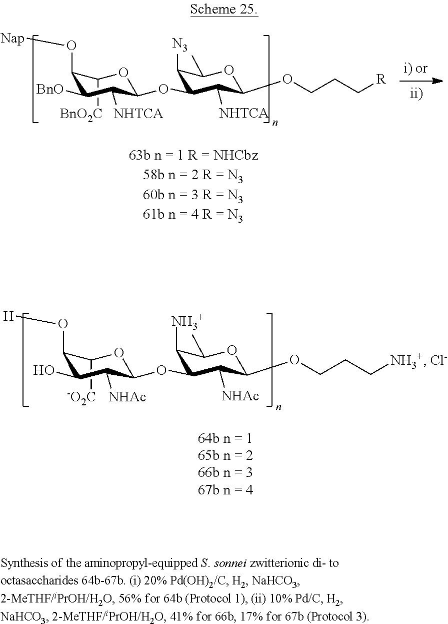

3-Aminopropyl (2-acetamido-2-deoxy-α-L-altropyranosyluronic acid)-(1→3)-2-acetamido-4-amino-2,4,6-trideoxy-β-D-galactopyranoside (64b). Disaccharide 63b was subjected to hydrogenation-mediated full deprotection (protocol 1). Thus, 20% Pd(OH)₂—C (180 mg, 3 equiv.) was added to the fully protected disaccharide 63b (100 mg, 86 μmol) in 2-MeTHF/isopropanol/water (1:15:3, v/v/v, 39 mL). The suspension was stirred vigorously for 1 h under a hydrogen atmosphere, 0.6 mM aq. NaHCO₃ (440 μL, 3 equiv.) was added. After stirring for another hour under a hydrogen atmosphere, more NaHCO₃ (150 μL, 1 equiv.) was added (twice) and the reaction was run for three more hours at which time LC-MS indicated reaction completion. Another equivalent of NaHCO₃ (150 μL, 1 equiv.) was added to reach a total of 6 equivalent. The suspension was filtered by passing through a 0.2 μm filter. Volatiles were evaporated and the residue was purified by C-18 chromatography eluting with water/ACN 100:0 to 90:10. The desired aminopropyl disaccharide 64b was obtained as a white lyophilized powder (23 mg, 48 μmol, 56%). The free disaccharide 64b had RP-HPLC (215 nm/ESLD): Rₜ=4.1/4.3 min (conditions A). ¹H NMR (D₂O) δ 4.85 (d, 1H, J_{1,2}=8.0 Hz, H-1_{A}), 4.41 (d, 1H, J_{1,2}=8.4 Hz, H-1_{B}), 4.34-4.31 (m, 2H, H-4_{A}, H-3_{B}), 3.98-3.85 (m, 5H, H-5_{A}, H-2_{A}, H-5_{B} H-2_{B}, OCH_{2Pr}), 3.71-3.66 (mₚₒ, 2H, H-3_{A}, OCH_{2Pr}), 3.42 (brs, 1H, H-4_{B}), 3.06 (t, 2H, J=6.8 Hz, NCH₂), 2.04, 1.99 (2s, 6H, CH_{3Ac}), 1.93 (t, J=6.0 Hz, CH₂), 1.28 (d, 3H, J_{5,6}=6.4 Hz, H-6_{B}). ¹³C NMR (D₂O) δ 175.2, 174.3 (NHCO_{A,B}), 174.2 (C-6_{A}), 101.8 (C-1_{B}, ¹J_{C,H}=161.2 Hz), 100.5 (C-1_{A}, ¹J_{C,H}=165.2 Hz), 78.3 (C-5_{A}), 77.2 (C-3_{B}), 69.7 (C-5_{B}), 69.0 (C-4_{A}), 68.3 (C-3_{A}), 68.0 (OCH_{2Pr}), 53.6 (C-4_{B}), 51.7 (C-2_{A}), 50.7 (C-2_{B}), 37.6 (NCH₂), 26.8 (CH_{2Pr}), 22.2 (2C, CH_{3Ac}), 15.8 (C-6_{B}). HRMS (ESI⁺): m/z [M+H]⁺ calcd for C₁₉H₃₅N₄O₁₀, 479.2348; found 479.2346.

3-Aminopropyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (65b). The azidopropyl tetrasaccharide 58b was submitted to full hydrogenation-mediated deprotection (protocol 1). The free tetrasaccharide 65b had RP-HPLC (215 nm/ELSD): Rₜ=5.0/5.2 min (conditions A). ¹H NMR (D₂O) δ 4.90 (d, 1H, J_{1,2}=8.0 Hz, H-1_{A}), 4.81-4.74 (mₚₒ, 2H, H-1_{A1}, H-1_{B}), 4.52-4.46 (mₚₒ, 3H, H-5_{A}, H-5_{A1}, H-1_{B1}), 4.40-4.36 (mₚₒ, 2H, H-4_{A}, H-4_{A1}), 4.18-4.16 (m, 2H, H-3_{B}, H-3_{B1}), 4.10-4.05 (m, 2H, H-5_{B}, H-5_{B1}), 4.01-3.99 (mₚₒ, 3H, H-4_{B}, H-2_{A}, OCH_{2Pr}), 3.90-3.80 (m, 4H, H-2_{A}, H-4_{B}, H-2_{B}, H-2_{B1}), 3.76-3.65 (mₚₒ, 4H, H-2_{B}, H-3_{A}, H-3_{A1}, OCH_{2Pr}), 3.11-3.07 (mₚₒ, 2H, NCH₂), 2.06, 2.03, 2.02, 1.98 (2s, 6H, CH_{3Ac}), 1.98-1.91 (mₚₒ, 2H, CH₂), 1.33 (dₚₒ, 6H, J_{5,6}=6.4 Hz, H-6_{B}, H-6_{B1}). ¹³C NMR (D₂O) δ 174.9, 174.7, 174.5, 174.4 (4C, NHCO_{A,B}), 174.1, 174.2 (2C, C-6_{A}, C-6_{A1}), 103.0 (C-1_{B1}, ¹J_{C,H}=166.0 Hz), 101.7 (C-1_{B}, ¹J_{C,H}=162.5 Hz), 101.1 (C-1_{A}, ¹J_{C,H}=165.0 Hz), 100.9 (C-1_{A}, ¹J_{C,H}=165.5 Hz), 78.0, 77.6 (2C, C-5_{A}, C-5_{A1}), 77.5 (C-4_{A}), 76.0, 75.7 (2C, C-3_{B}, C-3_{B1}), 69.3 (C-4_{A}), 67.5, 67.3 (2C, C-5_{B}, C-5_{B1}), 68.2 (OCH_{2Pr}), 68.2, 67.9 (2C, C-3_{A}, C-3_{A1}), 54.9, 54.8 (2C, C-4_{B}, C-4_{B1}), 51.6, 51.5 (2C, C-2_{A}, C-2_{A1}), 51.0, 50.9 (2C, C-2_{B}, C-2_{B1}), 37.6 (NCH₂), 26.7 (CH_{2Pr}), 22.5, 22.4 (4C, CH_{3Ac}), 15.7, 15.6 (2C, C-6_{B}, C-6_{B1}). HRMS (ESI⁺): m/z [M+H]⁺ calcd for C₃₅H₆₀N₇O₁₉ 882.3938, found 882.3929.

3-Aminopropyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (66b). Protocol 3: Hexasaccharide 60b (111 mg, 4.1 μmol) was solubilized in 2-MeTHF (2.0 mL) and diluted to 0.26 mM per repeating unit in 2-MeTHF/iPrOH/H₂O (1:10:1 v/v/v, 50 mL). The solution was degassed and 10% Pd/C (220 mg) was added. The suspension was stirred vigorously under a hydrogen atmosphere for 4 h, at which time 10% Pd/C (220 mg) and 1 M aq. NaHCO₃ (25 μL, 30 μmol, 7 equiv.) were added. After 40 h, 10% Pd/C (220 mg) was added and the suspension was stirred under a hydrogen atmosphere for 3 days, at which time RP-HPLC and HRMS controls revealed the presence of the desired compound and absence of any major chlorinated intermediate. The suspension was passed through a pad of Celite, and solids were washed extensively with water. Volatiles were evaporated and the aqueous phase was lyophilized, the residue solubilized in water (2.0 mL). pH was adjusted to 6 by addition of 1 M aq. NaHCO₃ and the was passed through a 0.2 μm filter and the filtrate was lyophilized. RP-HPLC purification (ACN in 0.08% TFA 0-18%) of the crude material gave the desired aminopropyl hexasaccharide 66b (22 mg, 17 μmol, 41%) as a white lyophilized powder. The free hexasaccharide 66b had RP-HPLC (215 nm/ELSD): Rₜ=6.6/6.7 min (conditions A), Rₜ=6.0/6.1 min (conditions E). HRMS (ESI⁺): m/z [M+H]⁺ calcd for C₅₁H₈₅N₁₀O₂₈ 1285.5529; found 1285.5510, m/z [M+2H]²⁺ calcd for C₅₁H₈₆N₁₀O₂₈ 643.2801; found 643.2792.

3-Aminopropyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (67b). Protocol 3: Octasaccharide 61b (77 mg, 2.2 μmol) was solubilized in 2-MeTHF (1.5 mL) and diluted to 0.19 mM per repeating unit in 2-MeTHF/iPrOH/H₂O (1:10:1 v/v/v, 27 mL). The solution was degassed and 10% Pd/C (164 mg, 7 equiv.) was added. The suspension was stirred vigorously under a hydrogen atmosphere for 4 h, at which time 10% Pd/C (164 mg, 7 equiv.) and 1 M aq. NaHCO₃ (18 μL, 18 μmol, 8 equiv.) were added. After 40 h, 10% Pd/C (164 mg, 7 equiv.) was added and the suspension was stirred under a hydrogen atmosphere for 3 days, at which time RP-HPLC and HRMS controls revealed the presence of the desired compound and of minor amounts of monochlorinated intermediates. The suspension was passed through a pad of Celite, and solids were washed extensively with water. Volatiles were evaporated and the aqueous phase was lyophilized. The residue solubilized in water (2.0 mL) was passed through a 0.2 μm filter and the filtrate was lyophilized. RP-HPLC purification (ACN in 0.08% TFA 0-18%) of the crude material (53 mg) in water (2.5 mL, pH 5) gave the desired aminopropyl octasaccharide 67b (6.5 mg, 3.8 μmol, 17%) as a white lyophilized powder. The free octasaccharide 67b had RP-HPLC (215 nm/ELSD): Rₜ=7.6/7.8 min (conditions A), Rₜ=6.2/6.4 min (conditions D). HRMS (ESI⁺): m/z [M+2H]²⁺ calcd for C₆₇H₁₁₁N₁₃O₃₇ 844.8596; found 844.8588, m/z [M+3H]³⁺ calcd for C₆₇H₁₁₂N₁₃O₃₇ 563.5755; found 563.5750.

### Example 12. Aminopropyl Linker Modification into Conjugation-Ready Oligosaccharides

Linker-Modification with SAMA or SPDP: Chemoselective Introduction of a Masked Thiol

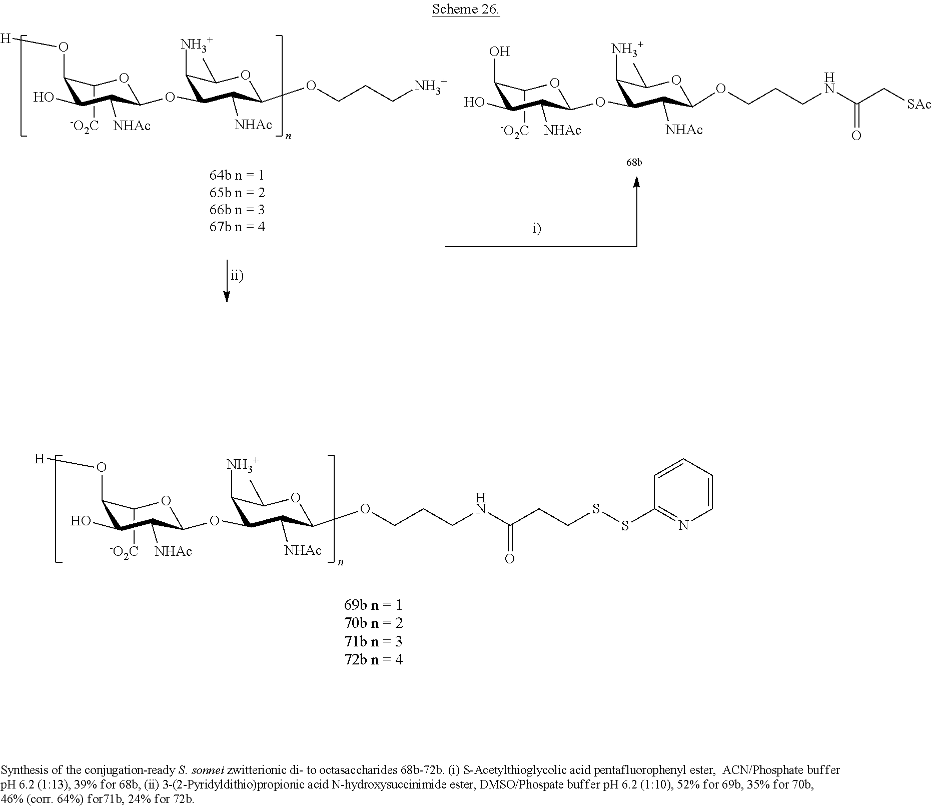

3-(2-Methylthioacetyl)ethylamido)-propyl (2-acetamido-2-deoxy-α-L-altropyranosyluronic acid)-(1→3)-2-acetamido-4-amino-2,4,6-trideoxy-β-D-galactopyranoside (68b). The aminopropyl oligosaccharide 64b (3.1 mg, 6.5 μmol, 1.0 equiv.) was dissolved in 0.1 M phosphate buffer pH 6.2 (800 μL). S-Acetylthioglycolic acid pentafluorophenyl ester (1.94 mg, 6.5 μmol, 1.0 equiv.) in ACN (60 μL) was added portionwise over 2 h to the reaction mixture stirred at rt. Progress was monitored by RP-HPLC and LCMS analysis. As some starting material was still visible after 2.5 h, the reaction was left at rt overnight. Purification by RP-HPLC (0 to 20% gradient over 20 min, UV detection at 230 nm) and repeated lyophilization of the pooled fractions of interest provided disaccharide 68b (1.5 mg, 39%) as a lyophilized powder. The desired linker equipped product disaccharide 68b had RP-HPLC (215 nm/ESLD): Rₜ=8.84/8.98 min (conditions D). ¹H NMR (D₂O) δ 4.87 (d, 1H, J_{1,2}=8.7 Hz, H-1_{A}), 4.50 (brs, 1H, H-5_{A}), 4.44 (d, 1H, J_{1,2}=8.6 Hz, H-1_{B}), 4.36 (brs, 1H, H-4_{A}), 4.12 (dd, 1H, J_{2,3}=10.4 Hz, J_{3,4}=4.0 Hz, H-3_{B}), 4.04 (qₚₒ, 1H, J_{5,6}=6.4 Hz, H-5_{B}), 3.98-3.92 (mₚₒ, 1H, H-2_{A}), 3.93 (brd, 1H, H-4_{B}), 3.89-3.83 (mₚₒ, 1H, OCH_{2Pr}), 3.81-3.76 (tₚₒ, 1H, H-2_{B}), 3.66 (ddₚₒ, 1H, H-3_{A}), 3.61 (brs, 2H, CH_{2,SAc}), 3.61-3.56 (mₚₒ, 2H, OCH_{2Pr}), 3.27-3.12 (m, 2H, NCH₂), 2.39 (SCH₃), 2.01, 1.98 (2s, 6H, CH_{3Ac}), 1.72 (tₚₒ, J=6.4 Hz, CH_{2Pr}), 1.28 (d, 3H, J_{5,6}=6.4 Hz, H-6_{B}). HRMS (ESI⁺): m/z [M+H]⁺ calcd for C₂₃H₃₉N₄O₁₂S, 595.2280; found 595.2270. HRMS (ESI⁺): m/z [M+Na]⁺ calcd for C₂₃H₃₈N₄O₁₂SNa 617.2099; found 617.2087.

General procedure for the introduction of the-(2-pyridyldithio)propionamide moiety: The aminopropyl oligosaccharide (1-10 mg, 1.0 equiv.) was dissolved in 0.1 M phosphate buffer pH 6.2 (1.0 mg/100-300 μL). 3-(2-Pyridyldithio)propionic acid N-hydroxysuccinimide ester (1.0 equiv.) in DMSO (˜1 mg in 10 μL) was added. The reaction mixture was stirred for 6-16 h at rt. Progress was monitored by RP-HPLC and LCMS analysis. At completion, the desired product was purified by RP-HPLC using a gradient of ACN in 0.08% aq. TFA as eluent. The product was confirmed based on NMR and HRMS analysis.

3-(3-(2-Pyridyldithio)propionamido)-propyl (2-acetamido-2-deoxy-α-L-altropyranosyluronic acid)-(1→3)-2-acetamido-4-amino-2,4,6-trideoxy-β-D-galactopyranoside (69b). Disaccharide 64b (15 mg, 31 μmol, 1.0 equiv.) dissolved in 0.1 M phosphate buffer pH 6.2 (3.1 mL) was subjected to regioselective amidation. SPDP (990 μg, 31 μmol, 1.0 equiv.) was added and the reaction mixture was stirred for 6 h. RP-HPLC of the crude material gave the amide 69b (5.1 mg, 24%) was obtained as a white lyophilized solid. The linker-equipped disaccharide 69b had RP-HPLC (215 nm/ELSD): Rₜ=11.9/12.1 min (conditions D). ¹H NMR (D₂O) δ 8.55 (d, 1H, J=5.2 Hz, H_{Ar}), 8.20 (t, 1H, J=4.0 Hz, H_{Ar}), 8.07 (d, 1H, J=8.4 Hz, H_{Ar}), 7.62 (t, 1H, J=6.4 Hz, H_{Ar}), 4.87 (d, 1H, J_{1,2}=8.4 Hz, H-1_{A}), 4.54 (brs, 1H, H-5_{A}), 4.45 (d, 1H, J_{1,2}=8.6 Hz, H-1_{B}), 4.36 (brs, 1H, H-4_{A}), 4.13 (dd, 1H, J_{2,3}=10.8 Hz, J_{3,4}=4.0 Hz, H-3_{B}), 4.02 (qₚₒ, 1H, J_{5,6}=6.4 Hz, H-5_{B}), 3.96 (tₚₒ, 1H, J=8.6 Hz, H-2_{A}), 3.91 (brd, 1H, H-4_{B}), 3.87-3.83 (mₚₒ, 1H, OCH₂), 3.77 (t, 1H, H-2_{B}), 3.67 (ddₚₒ, 1H, H-3_{A}), 3.60-3.54 (m, 1H, OCH₂), 3.21-3.09 (m, 2H, NCH₂), 3.11 (t, 2H, J=6.8 Hz, COCH₂), 2.65 (t, 2H, SCH₂), 2.00, 1.97 (2s, 6H, CH_{3Ac}), 1.71 (t, 2H, J=6.8 Hz, CH₂), 1.28 (d, 3H, J_{5,6}=6.4 Hz, H-6_{B}). ¹³C NMR (D₂O) δ 174.6, 174.0 (NHCO_{A,B}), 173.4 (NHCO), 173.1 (C-6_{A}), 156.7 (C_{Ar,q}), 144.7, 143.4, 124.4, 12.4 (C_{Ar}), 101.5 (C-1_{B}, ¹J_{C,H}=161.4 Hz), 101.0 (C-1_{A}, ¹J_{C,H}=165.2 Hz), 76.7 (C-5_{A}), 76.2 (C-3_{B}), 68.6 (C-4_{A}), 68.0 (OCH_{2Pr}), 67.9 (C-3_{A}), 67.3 (C-5_{B}), 54.7 (C-4_{B}), 51.4 (C-2_{A}), 50.8 (C-2_{B}), 36.2 (NCH₂), 34.5 (SCH₂), 34.2 (COCH₂), 28.3 (CH_{2Pr}), 22.2 (2C, CH_{3Ac}), 15.5 (C-6_{B}). HRMS (ESI⁺): m/z [M+H]⁺ calcd for C₂₇H₄₂N₅O₁₁S, 2676.2317; found 676.2292.

3-(3-(2-Pyridyldithio)propionamido)-propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (70b). The crude tetrasaccharide 65b (20 μmol theo. from 58b) was dissolved in 0.1 M phosphate buffer pH 6.2 (4.5 mL) and stirred vigorously at rt. A solution of SPDP (4.7 mg, 15 μmol, 0.75 equiv.) in DMSO (50 μL) was added portionwise over 2 h. After overnight stirring, more SPDP (4.7 mg, 15 μmol, 0.75 equiv.) in DMSO (50 μL) was added and the reaction mixture was stirred for another 6 h. One fourth of the total volume was purified by RP-HPLC. The amide 70b (5.1 mg, 24% from 58b) was obtained as a white lyophilized solid. The linker-equipped tetrasaccharide 70b had RP-HPLC (215 nm/ELSD): Rₜ=10.7/10.8 min (conditions E). ¹H NMR (D₂O) δ 8.56 (d, 1H, J=5.2 Hz, H_{Ar}), 8.23 (t, 1H, J=8.0 Hz, H_{Ar}), 8.09 (d, 1H, J=8.8 Hz, H_{Ar}), 7.65 (t, 1H, J=6.4 Hz, H_{Ar}), 4.87 (d, 1H, J_{1,2}=8.2 Hz, H-1_{A}), 4.78-4.75 (mₚₒ, 2H, H-1_{A1}, H-1_{B}), 4.63 (brs, 1H), 4.57 (brs, 1H, H-5_{A1}), 4.44-4.42 (mₚₒ, 2H, H-5_{A}, H-1_{B1}), 4.37 (dₚₒ, 1H, H-4_{A}), 4.16-4.10 (m, 2H, H-3_{B}), 4.04-4.00 (m, 2H, H-5_{B}, H-5_{B1}), 3.97-3.95 (mₚₒ, 1H, H-2_{A}), 3.91 (brs, 1H, H-4_{B}), 3.88-3.66 (mₚₒ, 7H, H-2_{A}, H-2_{B}, H-2_{B1}, H-3_{A}, H-3_{A1}, H-4_{B}, OCH_{2Pr}), 3.58-3.56 (mₚₒ, 1H, OCH_{2Pr}), 3.19-3.10 (mₚₒ, 4H, NCH₂, COCH₂₋ₗᵢₙₖₑᵣ), 2.64 (t, J=6.4 Hz, SCH₂), 2.06, 1.99, 1.95 (3s, 12H, CH_{3Ac}), 1.71-1.68 (mₚₒ, 2H, CH₂), 1.29 (dₚₒ, 6H, J_{5,6}=6.2 Hz, H-6_{B}). ¹³C NMR (D₂O) δ 174.6, 174.4, 173.9 (4C, NHCO_{A,B}), 173.3 (NHCOₗᵢₙₖₑᵣ), 172.9, 172.7 (C-6_{A}), 162.7 (C_{Ar,q}), 156.5, 144.3, 143.8, 124.5, 123.5 (C_{Ar}), 102.9 (C-1_{B1}, ¹J_{C,H}=166.4 Hz), 101.6 (C-1_{B}, ¹J_{C,H}=162.8 Hz), 101.0 (2C, C-1_{A}, C-1_{A1}, ¹J_{C,H}=166.0 Hz, 167.0 Hz), 76.8, 76.6 (3C, C-4_{A}, C-5_{A}, C-5_{A1}), 76.0, 75.9 (2C, C-3_{B}, C-1_{B1}), 68.5 (C-4_{A}), 68.0 (OCH_{2Pr}), 67.8, 67.6 (2C, C-5_{B}) C-1_{B1}), 67.4, 67.3 (2C, C-3_{A}, C-3_{A1}), 54.8, 54.7 (2C, C-4_{B}, C-4_{B1}), 51.5, 51.3 (2C, C-2_{A}, C-1_{A1}), 50.9, 50.8 (2C, C-2_{B}, C-1_{B1}), 36.2 (NCH₂), 34.4 (SCH₂), 34.2 (COCH₂), 28.3 (CH_{2Pr}), 22.3, 22.2 (4C, CH_{3Ac}), 15.6, 15.5 (2C, C-6_{B}, C-1_{B1}). HRMS (ESI⁺): m/z [M+H]⁺ calcd for C₄₃H₆₇N₈O₂₀S₂ 1079.3908, found 1079.3873.

3-(3-(2-Pyridyldithio)propionamido)-propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (71b). A solution of hexasaccharide 66b (5.1 mg, 3.97 μmol, 1.0 equiv.) in 0.1 M phosphate buffer pH 6.2 (1.0 mL) was stirred vigorously at rt and a solution of succinimidyl 3-(2-pyridyldithio)propionate (SPDP, 1.25 mg, 3.97 μmol, 1.0 equiv.) in DMSO (30 μL) was added to it portionwise (10 μL every 30 min). After stirring for 2 h at rt, more SPDP (0.4 mg, 0.3 equiv.) in DMSO (10 μL) was added and the reaction mixture was stirred for another 1.5 h at rt, then overnight at 4° C. RP-HPLC purification (0→40% ACN in 0.08% aq. TFA over 20 min) of the reaction mixture followed by lyophilisation gave the linker-equipped hexasaccharide 71b (2.7 mg, 1.82 μmol, 46%) and some remaining aminopropyl glycoside 66b (1.45 mg, 1.13 μmol) to reach a corrected yield of 64%. The desired 71b had RP-HPLC (215 nm/ELSD): Rₜ=11.3/11.4 min (conditions D). ¹H NMR (D₂O, 800 MHz) δ 8.56 (dd, 1H, J=5.8, <1.0 Hz, H_{Ar}), 8.23 (dt, 1H, J=8.0, 1.6 Hz, H_{Ar}), 8.07 (d, 1H, J=8.2 Hz, H_{Ar}), 7.64 (mₚₒ, 1H, J=6.4 Hz, H_{Ar}), 4.83 (d, 1H, J_{1,2}=8.3 Hz, H-1_{A2}), 4.73-4.66 (peaks overlap with HOD, 4H, H-1_{A1}, H-1_{A}, H-1_{B2}, H-1_{B1}), 4.62-4.60 (mₚₒ, 2H, H-5_{A1}, H-5_{A}), 4.54 (d, 1H, J_{4,5}=2.2 Hz, H-5_{A2}), 4.41 (tₚₒ, 1H, J_{3,4}=J_{4,5}=2.5 Hz, H-4_{A1}), 4.40 (tₚₒ, 1H, J_{3,4}=J_{4,5}=2.6 Hz, H-4_{A}), 4.38 (dₚₒ, 1H, J_{1,2}=8.5 Hz, H-1_{B}), 4.33 (tₚₒ, 1H, J_{3,4}=J_{4,5}=2.7 Hz, H-4_{A2}), 4.11-4.06 (mₚₒ, 3H, H-3_{B2}, H-3_{B1}, H-3_{B}), 4.00-3.96 (m, 3H, H-5_{B2}, H-5_{B1}, H-5_{B}), 3.93 (dd, 1H, J_{2,3}=10.6 Hz, J_{1,2}=8.4 Hz, H-2_{A2}), 3.87 (d, 1H, J_{3,4}=4.4 Hz, H-4_{B}), 3.83-3.75 (mₚₒ, 7H, H-2_{A1}, H-2_{A}, H-2_{B2}, H-2_{B1}, H-4_{B2}, H-41, OCH_{2Pr}), 3.72 (dd, 1H, J_{2,3}=10.8 Hz, J_{1,2}=8.8 Hz, H-2_{B}), 3.70-3.68 (mₚₒ, 2H, H-3_{A1}, H-3_{A}), 3.62 (dd, 1H, J_{2,3}=10.8 Hz, J_{3,4}=3.1 Hz, H-3_{A2}), 3.53-3.50 (mₚₒ, 1H, OCH_{2Pr}), 3.14-3.11 (m, 1H, NCH₂), 3.08-3.05 (mₚₒ, 1H, NCH₂), 3.07 (tₚₒ, 2H, J=6.6 Hz, COCH₂₋ₗᵢₙₖₑᵣ), 2.60 (t, 2H, J=6.6 Hz, SCH₂₋ₗᵢₙₖₑᵣ), 1.96, 1.95, 1.94, 1.91, 1.90 (6s, 18H, CH_{3Ac}), 1.68-1.63 (m, 2H, CH_{2Pr}), 1.26-1.24 (mₚₒ, 9H, H-6_{B2}, H-6_{B1}, H-6_{B}). ¹³C NMR (D₂O, 800 MHz) δ 174.6, 174.4, 174.4, 173.9, 173.3 (NHCO_{A,B}), 173.3 (NHCOₗᵢₙₖₑᵣ), 172.7, 172.5, 172.4 (C-6_{A}), 162.8 (C_{Ar,q}), 156.5, 144.1, 143.8, 124.6, 123.6 (C_{Ar}), 102.9 (C-1_{B2}, ¹J_{C,H}=165 Hz), 102.9 (C-1_{B1}, ¹J_{C,H}=166 Hz), 101.5 (C-1_{B}, ¹J_{C,H}=163 Hz), 101.1 (C-1_{A2}, ¹J_{C,H}=166 Hz), 101.0 (C-1_{A}, ¹J_{C,H}=166 Hz), 101.0 (C-1_{A1}, ¹J_{C,H}=168 Hz), 76.7, 76.6 (2C, C-4_{A}, C-4_{A1}), 76.5, 76.4 (3C, C-5_{A2}, C-5_{A1}, C-5_{A}), 76.0, 75.8 (3C, C-3_{B2}, C-3_{B1}, C-3_{B}), 68.4 (C-4_{A2}), 67.9 (OCH_{2Pr}), 67.8, 67.5 (3C, C-3_{A2}, C-3_{A1}, C-3_{A}), 67.3, 67.2 (3C, C-5_{B2}, C-5_{B1}, C-5_{B}), 54.8 (3C, C-4_{B2}, C-4_{B1}, C-4_{B}), 51.4, 51.2 (3C, C-2_{A2}, C-2_{A1}, C-2_{A}), 50.9, 50.8 (3C, C-2_{B2}, C-2_{B1}, C-2_{B}), 36.1 (NCH₂), 34.4 (SCH₂), 34.2 (COCH₂), 28.2 (CH_{2P}r), 22.2, 22.1 (6C, CH_{3Ac}), 15.5 (3C, C-6_{B}2, C-6_{B1}, C-6_{B}). HRMS (ESI⁺): m/z [M+2H]²⁺ calcd for C₅₉H₉₃N₁₁O₂₉S₂ 741.7786; found 741.7782. HRMS (ESI⁺): m/z [M+3H]³⁺ calcd for C₅₉H₉₄N₁₁O₂₉S₂ 494.8548; found 494.8546.

3-(3-(2-Pyridyldithio)propionamido)-propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (72b). A solution of octasaccharide 67b (5.1 mg, 3.0 μmol, 1.0 equiv.) in 0.1 M phosphate buffer pH 6.4 (1.0 mL) was stirred vigorously at rt and a solution of succinimidyl 3-(2-pyridyldithio)propionate (SPDP, 950 μg, 3.0 μmol, 1.0 equiv.) in DMSO (30 μL) was added to it portionwise (10 μL every 30 min). After stirring for 3 h at rt, more SPDP (90 μg, 0.3 equiv.) in DMSO (5.0 μL) was added and the reaction mixture was stirred for another 30 min at rt. RP-HPLC purification (0→40% ACN in 0.08% aq. TFA over 20 min) followed by lyophilisation gave the linker-equipped octasaccharide 72b (2.3 mg, 1.82 μmol, 41%) and some remaining aminopropyl glycoside 67b (2.0 mg, 1.16 μmol) to reach a corrected yield of 67%. The desired 72b had RP-HPLC (215 nm/ELSD): Rₜ=11.0/11.2 min (conditions D). ¹H NMR (D₂O, 800 MHz) δ 8.56 (d, 1H, J=5.8 Hz, H_{Ar}), 8.15 (ttₚₒ, 1H, J=8.0 Hz, H_{Ar}), 8.01 (d, 1H, J=8.4 Hz, H_{Ar}), 7.56 (ttₚₒ, 1H, J=6.0 Hz, H_{Ar}), 4.83 (d, 1H, J_{1,2}=8.4 Hz, H-1_{A3}), 4.74-4.66 (peaks overlap, 6H, H-1_{A}2, H-1_{A1}, H-1_{A}, H-1_{B}3, H-1_{B}2, H-1_{B1}), 4.56-4.55 (mₚₒ, 3H, H-5_{A2}, H-5_{A1}, H-5_{A}), 4.54 (brs, 1H, H-5_{A3}), 4.40-4.38 (mₚₒ, 4H, H-4_{A2}, H-4_{A1}, H-4_{A}, H-1_{B}), 4.33 (t, 1H, J_{3,4}=J_{4,5}=2.5 Hz, H-4_{A3}), 4.11-4.06 (mₚₒ, 4H, H-3_{B}3, H-3_{B2}, H-3_{B1}, H-3_{B}), 4.00-3.96 (mₚₒ, 4H, H-5_{B}3, H-5_{B2}, H-5_{B1}, H-5_{B}), 3.93 (dd, 1H, J_{2,3}=10.7 Hz, J_{1,2}=8.5 Hz, H-2_{A3}), 3.87 (d, 1H, J_{3,4}=4.4 Hz, H-4_{B}), 3.83-3.74 (mₚₒ, 10H, H-2_{A2}, H-2_{A1}, H-2_{A}, H-2_{B}3, H-2_{B2}, H-2_{B1}, H-4_{B}3, H-4_{B2}, H-4_{B1}, OCH_{2Pr}), 3.72 (dd, 1H, J_{2,3}=11.0 Hz, J_{1,2}=8.9 Hz, H-2_{B}), 3.69-3.67 (mₚₒ, 3H, H-3_{A2}, H-3_{A1}, H-3_{A}), 3.62 (dd, 1H, J_{2,3}=10.6 Hz, J_{3,4}=2.9 Hz, H-3_{A3}), 3.54-3.51 (mₚₒ, 1H, OCH_{2Pr}), 3.14-3.11 (m, 1H, NCH₂), 3.08-3.04 (mₚₒ, 1H, NCH₂), 3.06 (tₚₒ, 2H, J=6.3 Hz, COCH₂₋ₗᵢₙₖₑᵣ), 2.60 (t, 2H, J=6.4 Hz, SCH₂₋ₗᵢₙₖₑᵣ), 1.96, 1.95, 1.94, 1.92, 1.91, 1.90 (8s, 24H, CH_{3Ac}), 1.67-1.63 (m, 2H, CH₂), 1.26-1.24 (mₚₒ, 12H, H-6_{B3}, H-6_{B2}, H-6_{B1}, H-6_{B}). ¹³C NMR (D₂O, 800 MHz) δ 174.6, 174.4, 174.4, 174.1 (8C, NHCO_{A,B}), 173.3 (NHCOₗᵢₙₖₑᵣ), 173.2, 172.9, 172.4 (4C, C-6_{A}), 162.8 (C_{Ar,q}), 156.8, 144.8, 143.1, 124.0, 123.3 (C_{Ar}), 103.0 (C-1_{B3,} ¹J_{C,H}=165 Hz), 102.9 (C-1_{B2}, C-1_{B1}, ¹J_{C,H}=165 Hz, 166 Hz), 101.6 (C-1_{B}, ¹J_{C,H}=163 Hz), 101.1 (C-1_{A}3, ¹J_{C,H}=165 Hz), 101.0 (3C, C-1_{A}2, C-1_{A1}, C-1_{A}, ¹J_{C,H}=166 Hz, 168 Hz), 76.9 (3C, C-4_{A2}, C-4_{A1}, C-4_{A}), 76.8, 76.7 (4C, C-5_{A}3, C-5_{A2}, C-5_{A1}, C-5_{A}), 76.0, 75.7, 75.6 (4C, C-3_{B}3, C-3_{B2}, C-3_{B1}, C-3_{B}), 68.6 (C-4_{A3}), 68.2 (OCH_{2Pr}), 67.9, 67.8, 67.6 (4C, C-3_{A3}, C-3_{A2}, C-3_{A1}, C-3_{A}), 67.3, 67.2 (4C, C-5_{B}3, C-5_{B2}, C-5_{B1}, C-5_{B}), 54.8, 54.3 (4C, C-43, C-4_{B2}, C-4_{B1}, C-4_{B}), 51.4, 51.3 (4C, C-2_{A}3, C-2_{A2}, C-2_{A1}, C-2_{A}), 50.9, 50.8 (4C, C-2_{B}3, C-2_{B1}, C-2_{B1}, C-2_{B}), 36.1 (NCH₂), 34.4 (SCH₂), 34.0 (COCH₂), 28.2 (CH_{2Pr}), 22.2, 22.1 (8C, CH_{3Ac}), 15.5 (4C, C-6_{B}3, C-6_{B}2, C-6_{B1}, C-6_{B}). HRMS (ESI⁺): m/z [M+2H]²⁺ calcd for C₇₅H₁₁₈N₁₄O₃₈S₂ 943.3581; found 943.3567. HRMS (ESI⁺): m/z [M+3H]³⁺ calcd for C₇₅H₁₁₉N₁₄O₃₈S₂ 629.2412; found 629.2402.

### Example 13. Glycerol Aglycon Modification into Conjugation-Ready Oligosaccharides

Linker-Modification with PDPH: Chemoselective Introduction of a Masked Thiol by Means of an Aldehyde Intermediate

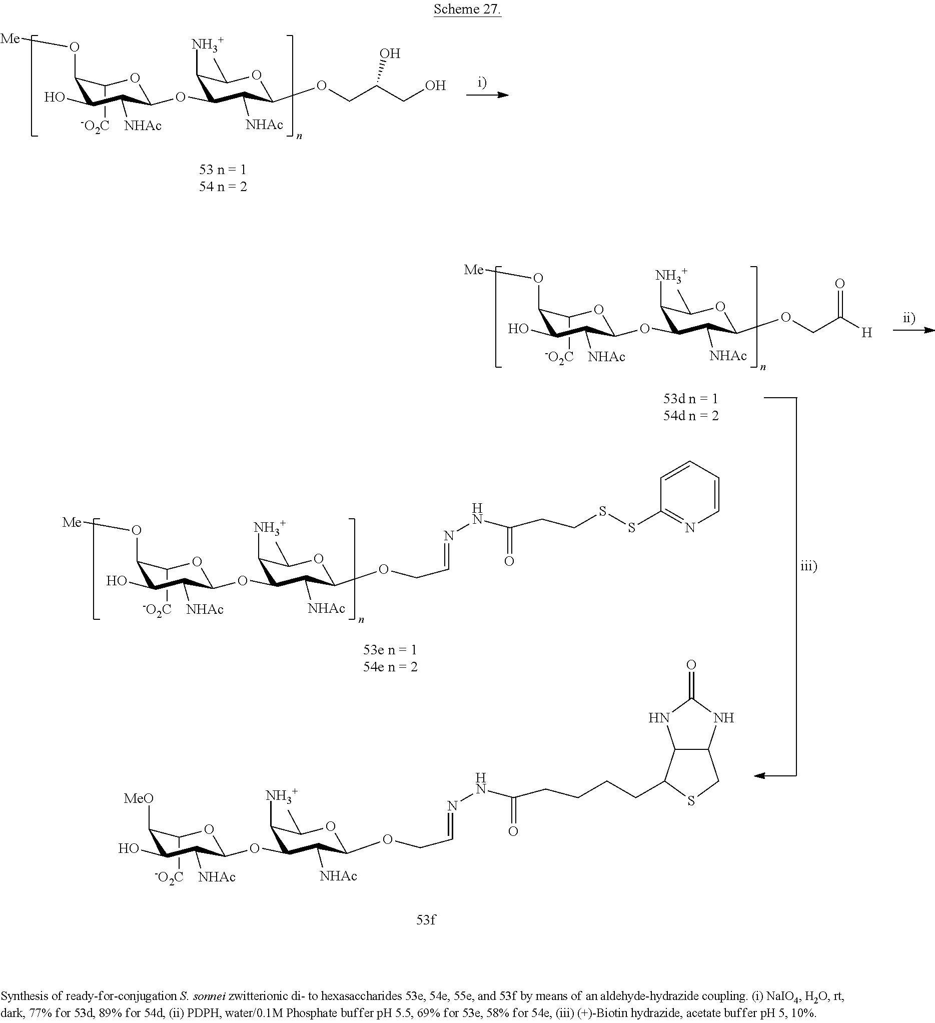

2-Oxoethyl (2-acetamido-2-deoxy-4-O-methyl-α-L-altropyranosyluronic acid)-(1→3)-2-acetamido-4-amino-2,4,6-trideoxy-β-D-galactopyranoside (53d). Diol 53 (10.8 mg, 21.2 μmol, 1.0 equiv.) was dissolved in water (2.6 mL) and aq. NaIO₄ (87 mM, 246 μL, 4.58 mg, 21.2 μmol, 1.0 equiv.) was added. The reaction mixture was stirred in the dark at rt and reaction progress was monitored by RP-HPLC. After 2 h, more aq. NaIO₄ (50 μL, 930 μg, 4.3 μmol, 0.2 equiv.) was added as some remaining starting 53 was still visible (RP-HPLC (215 nm): Rₜ=7.1 min (conditions A)) and stirring went on for 30 min at rt. The crude material was purified by RP-HPLC (0→70% ACN in water) to give the desired aldehyde 53d (7.9 mg, 16.5 μmol, 77%). Disaccharide 53d had RP-HPLC (215 nm): Rₜ=6.8 min (conditions A), Rₜ=5.9 min (conditions E). HRMS (ESI⁺): m/z [M+H]⁺ calcd for C₁₉H₃₉N₃O₁₁, 478.2031; found 478.2012.

2-(3-(2-Pyridyldithio)propanoylhydrazono)ethyl (2-acetamido-2-deoxy-4-O-methyl-α-L-altropyranosyluronic acid)-(1→3)-2-acetamido-4-amino-2,4,6-trideoxy-β-D-galactopyranoside (53e). Aldehyde 53d (5.1 mg, 10.7 μmol, 1.0 equiv.) was dissolved in water/0.1 M Phosphate buffer pH 5.5 (5:1, 1.2 mL) and 3-(2-pyridyldithio)propionyl hydrazide (PDPH, 4.9 mg, 21.4 μmol, 2 equiv.) in DMSO (20 μL) was stirred vigorously for 3 h at rt and reaction progress was monitored by RP-HPLC and LCMS. The crude material, showing MS (ESI⁺) for C₂₇H₄₀N₆O₁₁S₂Na m/z [M+H]+ 711.2, was purified by RP-HPLC (0→70% ACN in water) to give the desired hydrazone 53e (5.1 mg, 7.4 μmol, 69%). Linker-equipped disaccharide 53e had RP-HPLC (215 nm): Rₜ=10.0 min (conditions E). HRMS (ESI⁺): m/z [M+H]⁺ calcd for C₂₇H₄₁N₆O₁₁S₂ 689.2275, found 689.2270.

2-(Biotine-hydrazide)-ethyl (2-acetamido-2-deoxy-4-O-methyl-α-L-altropyranosyluronic acid)-(1→3)-2-acetamido-4-amino-2,4,6-trideoxy-β-D-galactopyranoside (53f). Diol 53 (2.0 mg, 3.9 μmol, 1.0 equiv.) was dissolved in acetate buffer pH 5 (1.3 mL) and a solution of aq. NaIO₄ (20 mM, 195 μL, 830 μg, 3.9 μmol, 1.0 equiv.) was added. The reaction mixture was stirred in the dark for 2 h at rt. Aq. KCl (1 M, 1.5 mg, 20 μL, 20 μmol, 5.1 equiv.) was added and the reaction mixture was stirred for an additional 20 min. (+)-Biotin hydrazide (2.0 mg, 8.2 μmol, 2.0 equiv.) was added and the mixture was stirred at rt for 48 h. The crude mixture was lyophilized and the residue was purified by RP-HPLC. The desired biotinylated disaccharide 53f (0.3 mg, 0.4 μmol, 10%) had HRMS (ESI⁺): m/z [M+H]⁺ calcd for C₂₉H₄₈N₇O₁₂S, 718.3076; found 718.3073.

2-Oxoethyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (54d). Diol 54 (9.2 mg, 10.1 μmol, 1.0 equiv.) was dissolved in water (1.5 mL) and aq. NaIO₄ (87 mM, 139 μL, 2.58 mg, 12.1 μmol, 1.2 equiv.) was added. The reaction mixture was stirred in the dark at rt and reaction progress was monitored by RP-HPLC (conditions A). After 2 h, the starting 54 (Rₜ=8.9 min) was absent and a new product was visible (Rₜ=8.4 min, MS (ESI⁺) for calcd for C₃₅H₅₇N₆O₂₀: m/z [M+H]+ 881.3). The crude material was purified by RP-HPLC (0→20% ACN in water) to give the desired aldehyde 54d (7.9 mg, 9.0 μmol, 89%). Tetrasaccharide 54d had RP-HPLC (215 nm): Rₜ=9.8 min (conditions A).

2-(3-(2-Pyridyldithio)propanoylhydrazono)ethyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (54e). Aldehyde 54d (4.3 mg, 4.9 μmol, 1.0 equiv.) was dissolved in water/0.1 M Phosphate buffer pH 7.5 (4:1, 1.0 mL) and PDPH (2.2 mg, 21.4 μmol, 2 equiv.) in DMSO (10 μL) was stirred vigorously for 3 h at rt and reaction progress was monitored by RP-HPLC and LCMS showing the starting material eluting at Rₜ=8.0 min (conditions A) and the product eluting at Rₜ=17.1 min (conditions A), Rₜ=9.9 min (conditions E), MS (ESI⁺) for C₄₃H₆₆N₉O₂₀S₂: m/z [M+H]+ 1092.2, m/z [M+2H]²+546.7. The crude material was purified by RP-HPLC (0→70% ACN in water) to give the desired hydrazone 54e (3.1 mg, 2.8 μmol, 5%). Linker-equipped tetrasaccharide 54e had RP-HPLC (215 nm): Rₜ=20.3 min (conditions A), Rₜ=12.2/13.2 min (conditions E, sample in water/ammonium acetate buffer pH 6.6). HRMS (ESI⁺): m/z [M+H]⁺ calcd for C₄₃H₆₆N₉O₂₀S₂1092.3860; found 1092.3860, m/z [M+2H]²⁺ calcd for C₄₃H₆₇N₉O₂₀S₂ 546.6966; found 546.6968.

### Example 14. Oligosaccharide-Protein Conjugates Exemplified for Tetanus Toxoid as the Carrier and the Thiol-Maleimide Conjugation Chemistry by Use of the SPDP Strategy Starting from (AB)ₙ Oligosaccharides Equipped with a Linker at their Reducing End

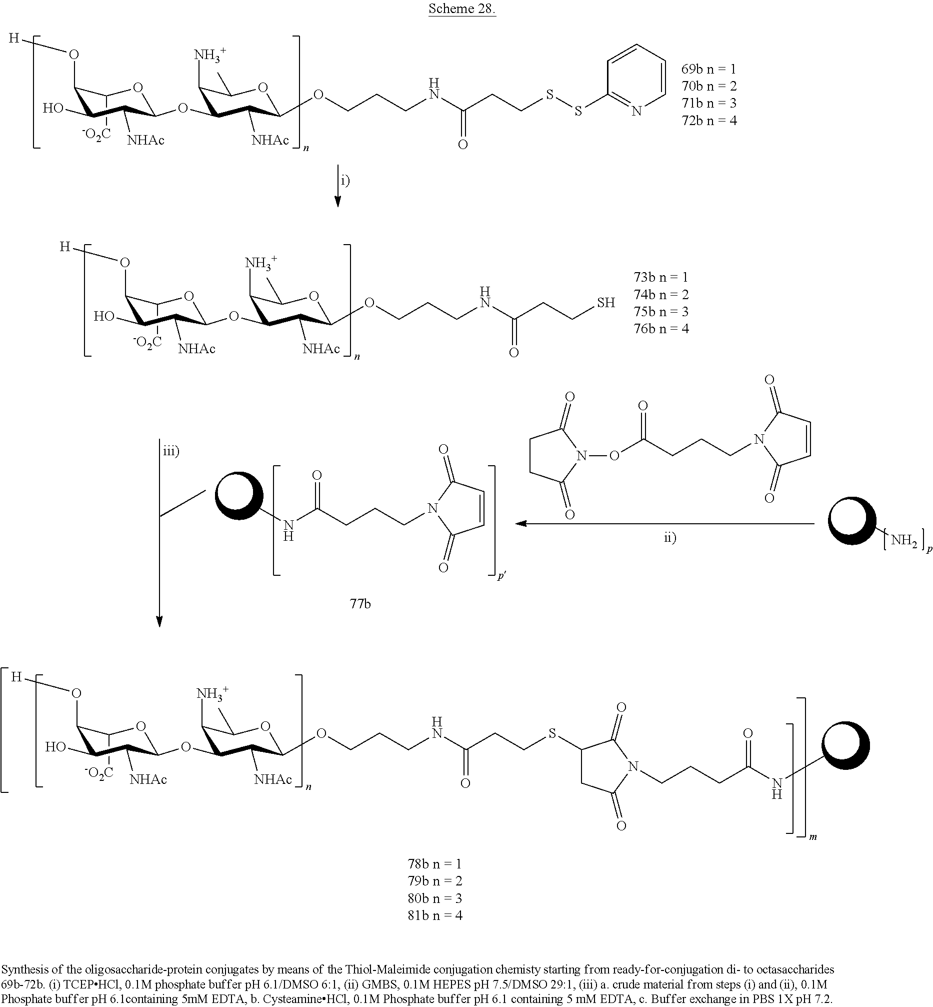

General Method for the Conjugation Step

Size Exclusion Chromatography (SEC). An ÄKTA pure chromatography system (GE Healthcare Life Sciences) was equipped with a high-resolution preparative gel Hiload 16/600 Superdex 200 μg column eluting with 0.2 μm sterile filtered phosphate buffered saline (PBS) xl, pH 7.2, 1.0 mL/min for preparative chromatography or with a Superdex 200 Increase 3.2/300 eluting with PBS xl, 0.05 mL/min for analytical chromatography.

Buffer exchange and concentration. Buffer exchange and concentration was performed using spin filters (Millipore, Amicon ultra 4 and 15) with a 30 kDa MWCO (15 min, 5,000 xg, rt). For each buffer exchange at least four consecutive cycles were performed, with an exchange ratio of at least 15 per cycle.

Protein concentration is estimated by UV detection (λ=280 nm) for tetanus toxoid conjugates (ε(TT)=ε=189,460 M⁻¹·cm⁻¹, mw(TT): 150,551 D) and CRM 197 conjugates ((ε(CRM)=ε=54,570 M⁻¹·cm⁻¹, mw(CRM): 58,413 D) with buffer as control

Protocol for MALDI analysis. The protein solution (20 μL) was passed through a ZipTip C4 and eluted on a MTP 384 ground steel target plate (Bruker-Daltonics, Germany) with 2 μL of 20 mg/mL sinapinic acid in 50% aq. ACN containing 0.1% aq. TFA as the matrix solution. Samples were air-dried for 15 min. Data were acquired on a Bruker UltrafleXtrem instrument, using the Flexcontrol software (Bruker-Daltonics, Germany). 10,000 shots were recorded in the positive ion linear mode in the m/z range of 30-210 kDa.

Disaccharide-TT conjugates (78b) from thiol-equipped disaccharide 73b. SEC-purified tetanus toxoid (TT, 150 kD, from Bio Farma (Bandung, Indonesia), 15.3 mg/mL, 228 μL, 3.49 mg) was buffer exchanged with 0.1 M HEPES pH 7.5 by passing through a 30 kD centrifugal filter four times. A solution of GMBS (1.04 mg, 3.71 μmol, 160 equiv.) in DMSO (15 μL) was added to the obtained solution of TT (23 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to reach a final concentration of intermediate 77b of 16.8 mg/mL.

Disaccharide 69b (1.65 mg, 2.4 μmol) was dissolved in 0.1 M phosphate buffer pH 6.1 (100 μL). A 11.4 μM solution of tris(2-carboxyethyl)phosphine hydrochloride (TCEP·HCl) in 0.1 M phosphate buffer pH 6.1 (10 μL, 672 μg, 2.34 μmol, 0.96 equiv.) was added and the solution was stirred at rt. Monitoring was achieved by RP-HPLC (conditions D) and HRMS revealing the absence of the starting 69b (Rₜ=10.0 min) and the presence of a major product corresponding to the expected thiol 73b. The later had RP-HPLC (215 nm/ELSD): Rₜ=8.3 min (conditions E). Thiol 73b was found to dimerize upon storage to have HRMS (ESI⁺): m/z [M+H]⁺ calcd for C₄₄H₇₅N₈O₂₂S₂ 1131.4437; found 1131.4430.

Two individual portions of the stock solutions of intermediates 77b and 73b, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. Volumes of harvest were adjusted to a volume of 1.0 mL. The final conjugates were analyzed by UV (λ=280 nm) and MALDI-MS for conjugation yield and carbohydrate:protein ratio.

Conjugate 78b1: From the stock solution of modified TT (77b, 89 μL, 1.5 mg, 10 nmol) and crude thiol 73b (27 μL, 404 μg, 185 nmol, 60 equiv.).

Conjugate 78b2: From the stock solution of modified TT (77b, 89 μL, 1.5 mg, 10 nmol) and crude thiol 73b (18 μL, 269 μg, 530 nmol, 40 equiv.).

Conjugate 78b3: From the stock solution of modified TT (77b, 89 μL, 1.5 mg, 10 nmol) and crude thiol 73b (18 μL, 269 μg, 530 nmol, 30 equiv.).

Tetrasaccharide-TT conjugates (79b) from thiol-equipped tetrasaccharide 74b. SEC-purified tetanus toxoid (TT, 150 kD, 1.9 mL, 6.17 mg/mL) was buffer exchanged with 0.1 M HEPES pH 7.5 by passing through a 30kD centrifugal filter four times and finally concentrated to 860 μL (final concentration of TT: 13.2 mg/mL). A solution of GMBS (3.4 mg, 12.1 μmol, 160 equiv.) in DMSO (30 μL) was added to the obtained solution of TT (88 μM, 76 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 810 μL to reach a final concentration of intermediate 77b of 13.78 mg/mL.

Tetrasaccharide 70b (3.68 mg, 3.41 μmol) was dissolved in 0.1 M phosphate buffer pH 6.1 (300 μL). A 64 mM solution of tris(2-carboxyethyl)phosphine hydrochloride (TCEP·HCl) in DMSO (53 μL, 980 μg, 3.41 μmol, 1.0 equiv.) was added and the solution was stirred at rt. Monitoring was achieved by RP-HPLC (conditions D/E) and HRMS revealing the absence of the starting 70b (Rₜ=11.2/10.7 min) and the presence of a major product corresponding to the expected thiol 74b. The later had RP-HPLC (215 nm): Rₜ=8.6 min (conditions D), Rₜ=8.3 min (conditions E). HRMS (ESI⁺): m/z [M+H]⁺ calcd for C₃₈H₆₃N₇O₂₀S, 970.3921; found 970.3917. HRMS (ESI⁺): m/z [M+Na]⁺ calcd for C₃₈H₆₂N₇O₂₀SNa 992.3741; found 992.3735.

Two individual portions of the stock solutions of intermediates 77b and 74b, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. Volumes of harvest were adjusted to a volume of 1.0 mL. The final conjugates were analyzed by UV (λ=280 nm) and MALDI-MS for conjugation yield and carbohydrate:protein ratio.

Conjugate 79b1: From the stock solution of modified TT (77b, 145 μL, 2.0 mg, 13.3 nmol) and crude thiol 74b (29.5 μL, 287 nmol, 21.6 equiv.).

Conjugate 79b2: From the stock solution of modified TT (77b, 72.6 μL, 1.0 mg, 6.6 nmol) and crude thiol 74b (27.1 μL, 264 nmol, 40 equiv.).

Conjugate 79b3: From the stock solution of modified TT (77b, 72.6 μL, 1.0 mg, 6.6 nmol) and crude thiol 74b (40.1 μL, 396 nmol, 60 equiv.).

As part of another experiment to reach a final concentration of intermediate 77b of 14.94 mg/mL, conjugates 79b4 and 79b5 were obtained.

Conjugate 79b4: From the stock solution of modified TT (77b, 89 μL, 1.3 mg, 8.7 nmol) and crude thiol 74b (31 μL, 261 nmol, 30 equiv.).

Conjugate 79b5: From the stock solution of modified TT (77b, 89 μL, 1.3 mg, 8.7 nmol) and crude thiol 74b (remaining stock).

Hexasaccharide-TT conjugates (80b) from thiol-equipped hexasaccharide 75b. SEC-purified tetanus toxoid (TT, 150 kD, 1.9 mL, 6.17 mg/mL) was buffer exchanged with 0.1 M HEPES pH 7.5 by passing through a 30 kD centrifugal filter four times and finally concentrated to 860 μL (final concentration of TT: 13.2 mg/mL). A solution of GMBS (3.4 mg, 12.1 μmol, 160 equiv.) in DMSO (30 μL) was added to the obtained solution of TT (88 μM, 76 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 810 μL to reach a final concentration of intermediate 77b of 13.78 mg/mL.

Hexasaccharide 71b (2.8 mg, 1.89 μmol) was dissolved in 0.1 M phosphate buffer pH 6.1 (189 μL). A 64 mM solution of tris(2-carboxyethyl)phosphine hydrochloride (TCEP·HCl) in DMSO (30 μL, 540 μg, 1.89 μmol, 1.0 equiv.) was added and the solution was stirred at rt. After 1 h, Monitoring by RP-HPLC (conditions D) and HRMS revealing the absence of the starting 71b (Rₜ=11.3 min) and the presence of a major product corresponding to the expected thiol 75b. The later had RP-HPLC (215 nm): Rₜ=8.6 min (conditions D). HRMS (ESI⁺): m/z [M+2H]²⁺ calcd for C₅₄H₉₀N₁₀O₂₉S, 687.2792; found 687.2792. HRMS (ESI⁺): m/z [M+H+Na]²⁺ calcd for C₅₄H₈₉N₁₀O₂₉SNa 698.2702; found 698.2699.

Three individual portions of the stock solutions of intermediates 77b and 75b, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. Volumes of harvest were adjusted to a volume of 1.0 mL. The final conjugates were analyzed by UV (λ=280 nm) and MALDI-MS for conjugation yield and carbohydrate:protein ratio.

Conjugate 80b1: From the stock solution of modified TT (77b, 72.6 μL, 1.0 mg, 6.6 nmol) and crude thiol 75b (19.2 μL, 166 nmol, 25 equiv.).

Conjugate 80b2: From the stock solution of modified TT (77b, 65.3 μL, 0.9 mg, 6.0 nmol) and crude thiol 75b (27.7 μL, 239 nmol, 40 equiv.).

Conjugate 80b3: From the stock solution of modified TT (77b, 65.3 μL, 1.0 mg, 6.0 nmol) and crude thiol 75b (46.2 μL, 398 nmol, 60 equiv.).

Octasaccharide-TT conjugates (81b) from thiol-equipped octasaccharide 76b. SEC-purified tetanus toxoid (TT, 150 kD, 1.9 mL, 6.17 mg/mL) was buffer exchanged with 0.1 M HEPES pH 7.5 by passing through a 30 kD centrifugal filter four times and finally concentrated to 860 μL (final concentration of TT: 13.2 mg/mL). A solution of GMBS (3.4 mg, 12.1 μmol, 160 equiv.) in DMSO (30 μL) was added to the obtained solution of TT (88 μM, 76 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 810 μL to reach a final concentration of intermediate 77b of 13.78 mg/mL.

Octasaccharide 72b (1.0 mg, 530 μmol) was dissolved in 0.1 M phosphate buffer pH 6.1 (100 μL). A 64 mM solution of tris(2-carboxyethyl)phosphine hydrochloride (TCEP·HCl) in DMSO (8.5 μL, 152 μg, 530 nmol, 1.0 equiv.) was added to reach a concentration of 4.9 nM of oligosaccharide and the solution was stirred at rt. After 1 h, Monitoring by RP-HPLC (conditions D) and HRMS revealing the absence of the starting 72b (Rₜ=11.1 min) and the presence of a major product corresponding to the expected thiol 76b. The later had RP-HPLC (215 nm): Rₜ=8.5 min (conditions D). HRMS (ESI⁺): m/z [M+2H]²⁺ calcd for C₇₀H₁₁₅N₁₃O₃₈ 888.8588; found 888.8589. HRMS (ESI⁺): m/z [M+H+Na]²⁺ calcd for C₇₀H₁₁₄N₁₃O₃₈SNa, 899.8498; found 899.8496. HRMS (ESI⁺): m/z [M+2Na]²⁺ calcd for C₇₀H₁₁₃N₁₃O₃₈SNa₂ 910.8407; found 910.8409.

Different individual portions of various stock solutions of intermediates 77b and 76b, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. Volumes of harvest were adjusted to a volume of 1.0 mL. The final conjugates were analyzed by UV (λ=280 nm) and MALDI-MS for conjugation yield and carbohydrate:protein ratio.

Conjugate 81b1: From the stock solution of modified TT (77b, 65.3 μL, 0.9 mg, 6.0 nmol) and crude thiol 76b (24.4 μL, 119 nmol, 20 equiv.).

Conjugate 81b2: From the stock solution of modified TT (77b, 65.3 μL, 0.9 mg, 6.0 nmol) and crude thiol 76b (48.9 μL, 239 nmol, 40 equiv.).

As part of another experiment to reach a final concentration of intermediate 77b of 13.85 mg/mL, conjugates 81b3-83b6 were obtained.

Conjugate 81b3: From the stock solution of modified TT (77b, 75 μL, 1.0 mg, 6.6 nmol) and crude thiol 76b (24 μL, 104 nmol, 15 equiv.).

Conjugate 81b4: From the stock solution of modified TT (77b, 319 μL, 4.4 mg, 29.3 nmol) and crude thiol 76b (160 μL, 705 nmol, 24 equiv.).

Conjugate 81b5: From the stock solution of modified TT (77b, 255 μL, 3.5 mg, 23.5 nmol) and crude thiol 76b (170 μL, 750 nmol, 32 equiv.).

Conjugate 81b6: From the stock solution of modified TT (77b, 28 μL, 392 μg, 2.6 nmol) and crude thiol 76b (23.6 μL, 104 nmol, 32 equiv.).

As part of another experiment to reach a final concentration of intermediate 77b of 12.8 mg/mL, conjugate 81b7 was obtained.

Conjugate 81b7: From the stock solution of modified TT (77b, 936 μL, 12.0 mg, 797 nmol) and crude thiol 76b obtained from precursor 72b (6.0 mg, 3.18 μmol, 40 equiv.) featuring a masked thiol moiety.

### Example 15. Oligosaccharide-Protein Conjugates Exemplified for Tetanus Toxoid as the Carrier and the Thiol-Maleimide Conjugation Chemistry by Use of the PDPH Strategy

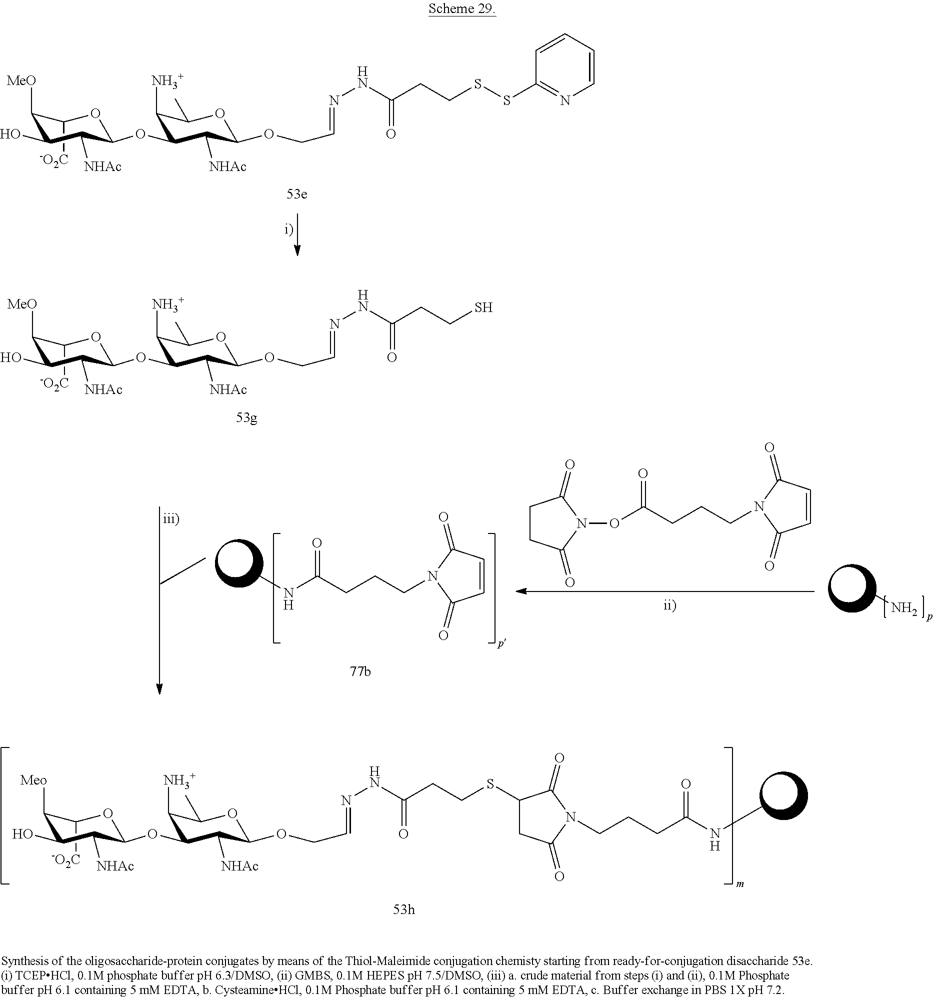

Disaccharide-TT conjugates (53h) from thiol-equipped disaccharide 53e. SEC-purified tetanus toxoid (TT, 150 kD, 6.12 mg/mL, 900 μL, 5.5 mg) in PBS 1× was buffer exchanged with 0.1 M HEPES pH 7.5 by passing through a 30 kD centrifugal filter four times to reach a concentration of 10.9 mg/mL (550 μL). A solution of GMBS (1.5 mg, 5.35 μmol, 145 equiv.) in DMSO (15 μL) was added to the obtained solution of TT (32.7 nmol). Modification was performed at ambient temperature for 1 h. After this time, the entire volume of intermediate 77b was buffer exchanged four times with 0.1 M Phosphate buffer pH 6.3 containing 5 mM EDTA, and finally concentrated to reach a final concentration of intermediate 77b of 14.2 mg/mL (380 μL).

Disaccharide 53e (4.6 mg, 6.67 μmol) was dissolved in 0.1 M phosphate buffer pH 6.1 (165 p L). A 20 mM solution of TCEP·HCl in 0.1 M phosphate buffer pH 6.3 (335 μL, 1.91 mg, 6.66 μmol, 1.0 equiv.) was added and the solution was stirred at rt for 1.5 h. Monitoring was achieved by RP-HPLC (conditions E) and LCMS revealing the absence of the starting 53e (Rₜ=10.0 min) and the presence of a major product (Rₜ=6.0 min) corresponding to the expected thiol 53g as revealed by MS (ESI⁺) for C₂₂H₃₇N₅O₁₁S: m/z [M+H]+ 580.2, m/z [M+Na]⁺ 602.2. The stock solution corresponding to a total of 6.7 μmol in 500 μL was kept at 0° C. before use.

Four individual portions of the stock solutions of intermediates 77b and 53g, respectively, were used to obtain conjugates with different carbohydrate:protein ratios. The expected amounts of each one of the two solutions were mixed and gently stirred for 3.5 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. Volumes of harvest were adjusted to a volume of 1.0 mL. The final conjugates were analyzed by UV (λ=280 nm) and MALDI-MS for conjugation yield and carbohydrate:protein ratio.

Conjugate 53h1: From the stock solution of modified TT (77b, 70 μL, 1.0 mg, nmol) and crude thiol 53g (15 μL, 115 μg, 199 nmol, 30 equiv.).

Conjugate 53h2: From the stock solution of modified TT (77b, 70 μL, 1.0 mg, nmol) and crude thiol 53g (30 μL, 230 μg, 530 nmol, 60 equiv.)

Conjugate 53h3: From the stock solution of modified TT (77b, 70 μL, 1.0 mg, nmol) and crude thiol 53g (twice 15 μL, 230 μg, 530 nmol, 60 equiv.).

Conjugate 53h4: From the stock solution of modified TT (77b, 89 μL, 1.0 mg, nmol) and crude thiol 53g (thrice 15 μL, 345 μg, 597 nmol, 90 equiv.).

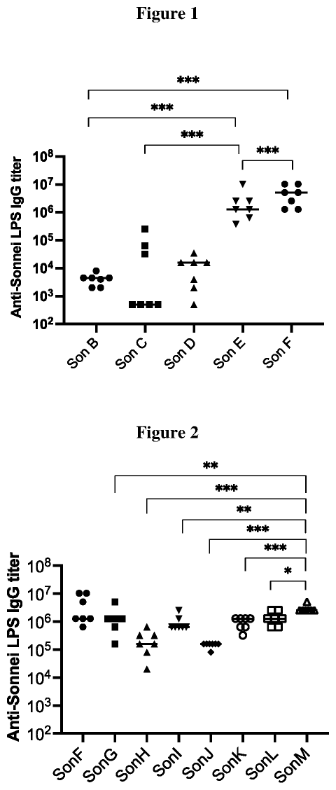

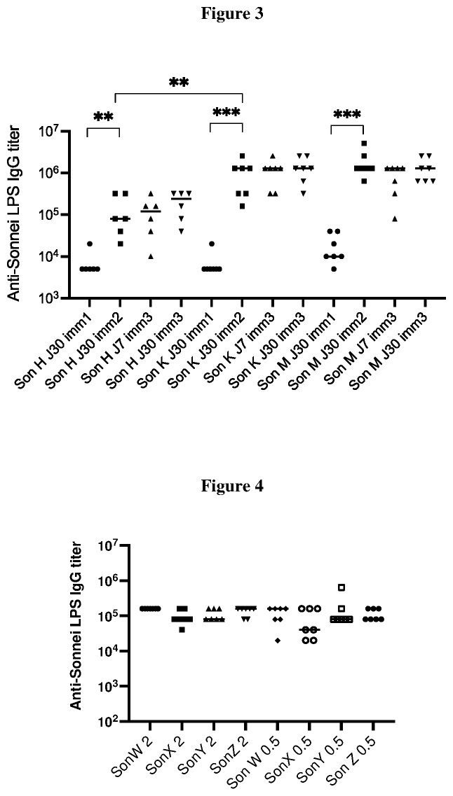

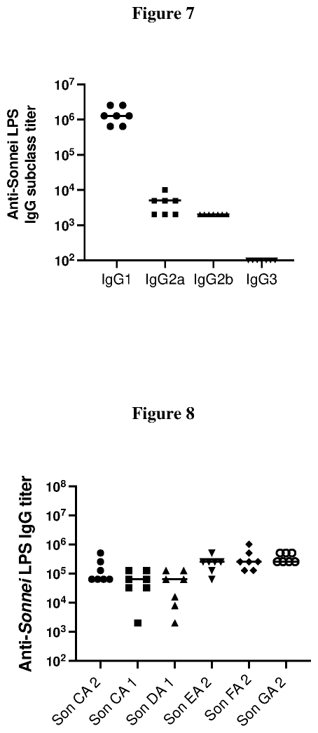

TABLE 1
S. sonnei (AB)ₙ oligosaccharide-TT conjugates obtained
by means of the thiol-maleimide chemistry.

| | | | Average | | | | |
|---|---|---|---|---|---|---|---|
| | Code in | Conjugate | OS:TT ratio | TT conc^{a} | OS conc | Total | |
| | FIGS. 1-7 | number | (m) | mg/mL | μg/mL | volume | |
| Ssonnei | | 53h1 | 4.7 | 1.68 | | | |
| SS-TT2 | | 53h2 | 7.5 | 1.65 | | | |
| | | 53h3 | 4 | 0.89 | | | |
| | | 53h4 | 7.3 | 0.64 | | | |
| | Son D | 78b1 | 30 | 1.81 | 145 | 800 | μL |
| | Son C | 78b2 | 23 | 1.66 | 100 | 1.1 | mL |
| | Son B | 78b3 | 13 | 1.59 | 56 | 870 | μL |
| Ssonnei | Son E | 79b4 | 8.5 | 1.31 | 59 | 1.06 | mL |
| SS-TT4 | Son F | 79b5 | 12 | 1.58 | 95 | 1.01 | mL |
| | Son G | 79b1 | 11 | 2.03 | 100 | 920 | μL |
| | Son H | 79b3 | 19 | 1.01 | 101 | 720 | μL |
| | Son N | 79b2 | 13 | 2.27 | 158 | 875 | μL |
| Ssonnei | Son I | 80b1 | 8.5 | 1.11 | 75 | 880 | μL |
| SS-TT6 | Son J | 80b2 | 15 | 0.99 | 120 | 905 | μL |
| | Son K | 80b3 | 19 | 0.94 | 145 | 580 | μL |
| Ssonnei | Son L | 81b1 | 5.5 | 0.96 | 56 | 880 | μL |
| SS-TT8 | Son M | 81b2 | 13 | 0.88 | 120 | 730 | μL |
| | Son W | 81b3 | 7 | 1.05 | 80 | 910 | μL |
| | Son X | 81b4 | 12.5 | 4.05 | 560 | 1150 | μL |
| | Son Y | 81b5 | 13.5 | 2.75 | 400 | 1170 | μL |
| | Son Z | 81b6 | 19 | 0.71 | 150 | 540 | μL |
| | Son AA | 81b7 | 10 | 3.15 | 340 | 3.86 | mL |
| Ssonnei | Son BA | 82b1 | 6 | 2.15 | 175 | 800 | μL |
| SS-TT10 | | | | | | | |

### Example 16: Study in Mice

Mice Immunization

For each of the adjuvanted conjugates, seven week-old Balb/c female mice (Janvier Labs, France) were immunized intramuscularly (i.m.) with amounts of conjugates corresponding to 2.5, 2.0, 1.0 or 0.5 μg equivalent of oligosaccharide per dose depending on the experiments, adjuvanted with aluminium hydroxide (alum, AlH, Alhydrogel, Brenntag, Denmark) unless stated. Alum was used at a concentration of 1.4 mg/mL in Tris pH 7.2 20 mM, and mixed v/v with the conjugates, resulting to a dose of 143 μg per mouse/per injection. After 5 min incubation at rt, 200 μL of the adjuvanted glycoconjugates were injected at two sites (100 μL at each site). Three immunizations were performed at 3 week-interval. Blood samples were recovered one week after the third injection unless stated. For some experiments, kinetics was performed with blood samples recovered 3 weeks after each injection. Seven mice were used per group.

Measurement of the Anti-S. sonnei IgG Response

The glycoconjugate-induced anti-LPS IgG response specific for S. sonnei LPS was measured by ELISA using purified S. sonnei LPS purified from the S. sonnei reference strain (CIP 106 347) as previously described.^{[2]} Briefly, 2.5 μg of purified S. sonnei LPS was coated per ELISA plate well in PBS and incubated at 4° C. overnight. After washing the wells with PBS-Tween 20 0.01%, saturation was performed by incubating the plate for 30 min at 37° C. with PBS-BSA 1%. Then, serial dilutions of mouse sera in PBS-BSA 1% were incubated for 1 h at 37° C. After washing with PBS-Tween 20 0.01%, anti-mouse IgG peroxidase-labeled conjugate (Sigma-Aldrich) was used as secondary antibody at a dilution of 1/5,000. The IgG titer was defined as the last dilution of serum giving rise to twice the OD value obtained with similarly diluted pre-immune serum. To measure the anti-S. sonnei LPS IgG subclasses, a similar ELISA was performed except that anti-mouse IgG1, IgG2a, IgG2b and IgG3 peroxidase-labeled conjugates (Sigma-Aldrich) were used as secondary antibody at a dilution of 1/5,000.

In addition to the original TT-conjugates featuring an (AB)ₙ hapten (n=1-4), it is noted that other TT-conjugate featuring an (AB)ₙ hapten (n=4, 5) were successfully obtained by use of the same procedure.

### Example 17: Synthesis of Other (AB)ₙ Oligosaccharide TT-Conjugates of the Invention—Linker-Equipped Oligosaccharides Featuring a 4_{A}-Endchain Hydroxyl Group

Azidopropyl Aglycon as Linker Precursor—Post-Chain Elongation Linker Introduction

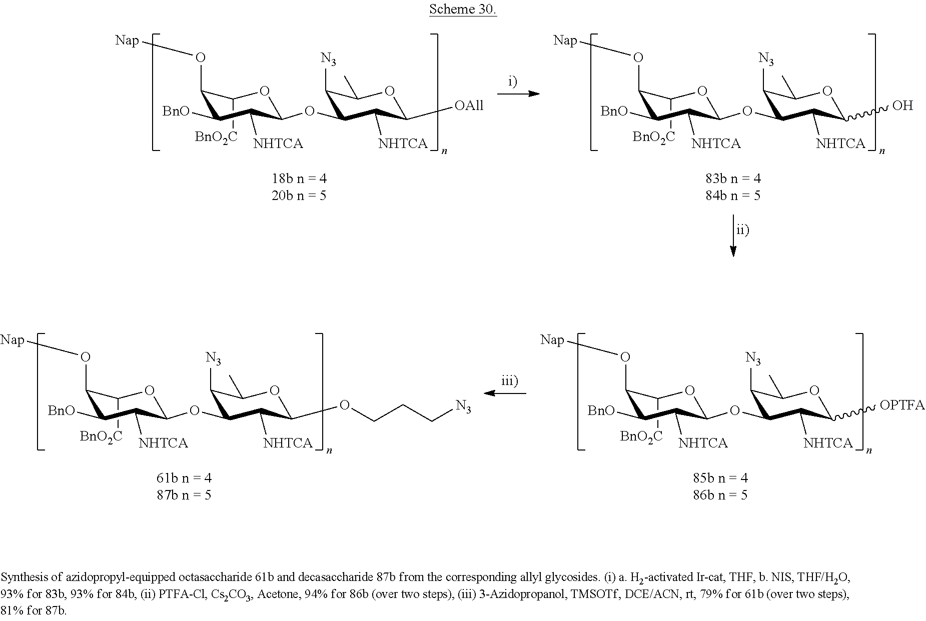

3-Azidopropyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (61b). Step 1: [Ir(COD)(PMePh₂)₂]⁺PF₆⁻ (29 mg, 35 μmol, 0.05 equiv.) in anhyd. THF (4 mL) was stirred for 45 min under an H₂ atmosphere. The pink color of the solution turned to yellow. The solution was degassed repeatedly and poured into a solution of allyl glycoside 18b (2.39 g, 693 μmol, 1.0 equiv.) in anhyd. THF (20 mL). The reaction was stirred for 8 h at rt. NIS (187 mg, 831 μmol, 1.2 equiv.) and H₂O (5 mL) were added, and the reaction stirred for another 4 h. Following a TLC analysis, 50% aq. Na₂S₂O₃ (10 mL) were added. The reaction mixture was concentrated under reduced pressure, and the aq. phase was diluted with DCM (50 mL) and water (50 mL). The DCM layer was washed with 50% aq. NaHCO₃ (50 mL) and brine (50 mL), dried over Na₂SO₄, and concentrated under reduced pressure. The crude was purified by flash chromatography (Tol/ACN, 80:20→75:25) to give hemiacetal 83b as a white solid (2.2 g, 645 μmol, 93%). Hemiacetal 83b had R_{f} 0.3, 0.4 (Tol/ACN 4:1). HRMS (ESI⁺): m/z [M+2NH₄]²⁺ calcd for C₁₃₁H₁₃₄Cl₂₄N₂₂O₃₇ 1729.0814; found 1729.0814.

Step 2: PTFACl (153 μL, 968 μmol, 1.5 equiv.) and Cs₂CO₃ (252 mg, 774 μmol, 1.2 equiv.) were added to a solution of the hemiacetal 83b (2.2 g, 645 μmol, 1.0 equiv.) in acetone (13 mL). The reaction mixture was stirred for 2 h at rt. Following a TLC analysis, the mixture filtered over a pad of Celite® and washed with acetone (2×5 mL). The combined filtrates were concentrated under reduced pressure. Flash chromatography (Tol/ACN, 80:20) of the crude gave a mix of the required PTFA donor 85b and of the corresponding oxazoline as a white solid. Donor 85b had R_{f} 0.55 (Tol/EtOAc 4:1). HRMS (ESI⁺): m/z [M+2NH₄]²⁺ calcd for C₁₃₉H₁₃₈Cl₂₄F₃N₂₃O₃₇ 1810.6028; found 1810.6024. The oxazoline had HRMS (ESI⁺): m/z [M+2NH₄]²⁺ calcd for C₁₃₁H₁₃₂Cl₂₄N₂₂O₃₆ 1720.0787; found 1720.0765.

Step 3: To a solution of the PTFA donor 85b (2.0 g, 558 μmol, 1.0 equiv.) in anhyd. DCE (10 mL) was added 3-azidopropanol (85 μL, 838 μmol, 1.5 equiv.) The solution was stirred with freshly activated MS 4 Å (˜1.0 g) under an argon atmosphere for 1 h. TMSOTf (7 μL, 39 μmol, 0.07 equiv. in anhyd. ACN (1 mL)) was added over 1 h while stirring the mixture at rt. After another 30 min at rt following a TLC analysis (Tol/ACN 4:1), Et₃N (1 equiv. vs TMSOTf) was added. The suspension was filtered over a fitted funnel and washed with DCM (3×5 mL). The combined filtrate was concentrated and the yellow residue purified by automated flash chromatography (SiOH 25 μm, Tol/ACN, 86:14). The azidopropyl glycoside 61b, obtained as a white solid (1.55 g, 443 μmol, 79%), had R_{f} 0.5 (Tol/ACN 4:1). HRMS (ESI⁺): m/z [M+2NH₄]²⁺ calcd for C₁₃₄H₁₃₉Cl₂₄N₂₅O₃₇ 1770.6082; found 1770.6086.

3-Azidopropyl (benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-4-O-(2-naphthylmethyl)-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-(4-azido-2-trichloroacetamido-2,4,6-trideoxy-β-D-galactopyranosyl)-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (87b). Step 1: [Ir(COD)(PMePh₂)₂]⁺PF₆⁻ (23 mg, 27 μmol, 0.05 equiv.) in anhyd. THF (3 mL) was stirred for 30 min under an H₂ atmosphere. The pink color of the solution turned to yellow. The mixture was degassed repeatedly with addition of argon. The solution transferred to a solution of allyl glycoside 20b (2.3 g, 540 μmol, 1.0 equiv.) in anhyd. THF (25 mL). The reaction was stirred at rt. After 16 h, NIS (146 mg, 647 μmol, 1.2 equiv.) and water (6 mL) were added. After stirring at rt for another 2 h, 50% aq. Na₂S₂O₃ (10 mL) was added. Volatiles were removed under reduced pressure. The residue was diluted with DCM (40 mL) and water (40 mL). The organic layer was separated, washed with 50% aq. NaHCO₃, and brine. The organic phase was collected, dried over Na₂SO₄, and concentrated under reduced pressure. Flash chromatography (Tol/EtOAc 70:30→60:40) of the crude gave hemiacetal 84b as a white solid (2.0 g, 474 μmol, 87%). Hemiacetal 84b had R_{f} 0.5, 0.6 (Tol/ACN 6:4). HRMS (ESI⁺): m/z [M+2NH₄]²⁺ calcd for C₁₆₁H₁₆₃Cl₃₀N₂₇O₄₆ 2133.0925; found 2133.0909.

Step 2: PTFACl (112 μL, 710 μmol, 1.5 equiv.) and Cs₂CO₃ (185 mg, 568 μmol, 1.2 equiv.) were added to a solution of hemiacetal 84b (2.0 g, 474 μmol, 1.0 equiv.) in acetone (10 mL). After stirring for 4 h at rt, the suspension was passed through a bed of Celite®, and washed with acetone (2×10 mL). The combined filtrates were concentrated. Flash chromatography (cHex/EtOAc 60:40) of the crude gave the PTFA donor 86b as a white solid (2.0 g, 455 μmol, 94%). Donor 86b had R_{f} 0.5, 0.6 (Tol/ACN 6:4). HRMS (ESI⁺): m/z [M+2NH₄]²⁺ calcd for C₁₆₉H₁₆₇Cl₃₀F₃N₂₈O₄₆ 2219.1060; found 2219.1055.

Step 3: A solution of PTFA donor 76_{B} (2.0 g, 455 μmol, 1.0 equiv.) and 3-azido propanol (126 μL, 1.36 mmol, 3.0 equiv.) in anhyd. DCE (22 mL) was stirred with freshly activated MS 4 Å (1.0 g) for 1 h at rt under an argon atmosphere. TMSOTf (6 μL, 32 μmol, 0.07 equiv.) in anhyd. ACN (1 mL) were added over 30 min to the reaction mixture at rt. After another 1 h at rt, a TLC analysis (Tol/ACN 4:1) showed reaction completion. Et₃N (1.0 equiv. vs TMSOTf) was added. The suspension was filtered over a Celite® bed, washed with DCM (2×10 mL). The combined filtrates were concentrated under reduced pressure. Flash chromatography (SiOH 25 μm, Tol/ACN 82:18) gave decasaccharide 87b as a white solid (1.6 g, 372 μmol, 81%). The azidopropyl glycoside 87b had R_{f} 0.5 (Tol/ACN 4:1). HRMS (ESI⁺): m/z [M+2NH₄]²⁺ calcd for C₁₆₄H₁₆₈Cl₃₀N₃₀O₄₆ 2174.1162; found 2174.1153.

Full Deprotection and Linker Modification with SPDP: Chemoselective Introduction of a Masked Thiol

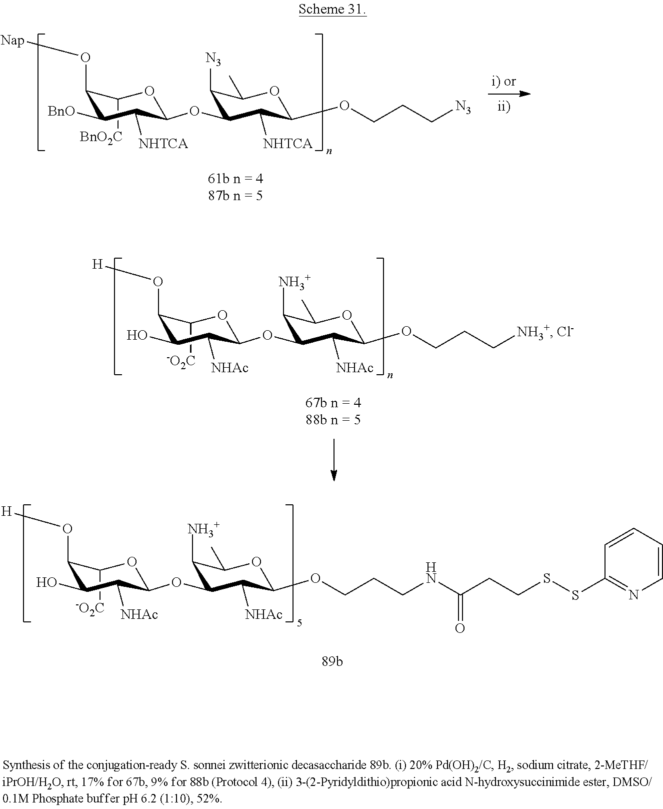

3-Aminopropyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (67b). Protocol 4. Isopropanol (60 mL) and water (30 mL) were added to a solution of octasaccharide 61b (100 mg, 29 μmol, 1.0 equiv.) in 2-MeTHF (4 mL). Sodium citrate monobasic (147 mg, 687 μmol, 24 equiv.) in water (2 mL) was added followed by addition of 20% Pd(OH)₂/C (320 mg, 458 μmol, 16 equiv.). The suspension was stirred under hydrogen for 18 h. NaHCO₃ (57 mg, 687 μmol, 24 equiv.) was added. The suspension was passed through a bed of Celite®, and washed with methanol (2×10 mL), and water (2×10 mL). Volatiles were removed under reduced pressure. The resulting solution (˜50 mL) was passed through a 0.2 μm syringe filter. Following lyophilization, the crude was purified by Sephadex G-10 gel filtration (100% Milli-Q water). Fractions corresponding to the desired product were lyophilized. Lastly, the residue was purified by semi-preparative RP-HPLC to give the desired octasaccharide 67b (8.6 mg, 5.1 μmol, 17%) as a lyophilized white powder. Analytical data were as above.

3-Aminopropyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (88b). Protocol 4. Isopropanol (120 mL) and water (40 mL) were added to decasaccharide 87b (200 mg, 46 μmol, 1.0 equiv.) in 2-MeTHF (8 mL), followed by sodium citrate monobasic (298 mg, 1.39 mmol, 30 equiv.) and 20% Pd(OH)₂/C (650 mg, 929 μmol, 20 equiv.). The suspension was stirred under a hydrogen atmosphere for 24 h at rt. NaHCO₃ (117 mg, 1.39 mmol, 30.0 equiv.) was added and the suspension was filtered over a bed of Celite®, washed with methanol (2×15 mL) and then water (2×15 mL). The combined filtrates were concentrated (˜60 mL) under reduced pressure. The solution was passed through a 0.2 μm syringe filter and lyophilized. The residue was purified by Sephadex G-10 gel filtration (conditions) and fractions corresponding to desired product were lyophilized. Semi-preparative RP-HPLC to give the decasaccharide 88b as a white solid (8.6 mg, 4.1 μmol, 9%). The aminopropyl glycoside 88b had RP-HPLC (215 nm/ELSD): Rₜ=8.3/8.4 min (conditions A). HRMS (ESI⁺): m/z [M+2H]²⁺ calcd for C₈₃H₁₃₆N₁₆O₄₆ 1046.4392; found 1046.4382. HRMS (ESI⁺): m/z [M+3H]³⁺ calcd for C₈₃H₁₃₇N₁₆O₄₆ 697.9619; found 697.9611.

3-(3-(2-Pyridyldithio)propionamido)-propyl (2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (89b). Phosphate buffer (0.4 mL, 0.2 M, pH 6.6) was added to a solution of the free decasaccharide 88b (3.0 mg, 1.4 μmol, 1.0 equiv.) in Milli-Q water (0.3 mL). The solution was cooled to 5° C. A solution of SPDP (1.8 mg, 5.7 μmol, 4.0 equiv.) in DMSO (15 μL) was added in three portions (⅓^{rd} of the total volume each). After adding the first portion to the cooled, stirring was pursued for 2 h while the reaction mixture reached rt. The second portion of SDDP solution (5.0 μL) was added. After stirring for another 5 h at rt, the remaining SPDP solution was added and the reaction was left overnight at rt. The reaction mixture was filtered through a 0.2 μm centrifuge filter and the filtrate was subjected to semi-preparative RP-HPLC. The target decasaccharide 89b (1.2 mg, 525 nmol, 36%) was isolated as a white solid. The linker equipped decasaccharide 89b had RP-HPLC (215 nm/ELSD): Rₜ=11.1/11.2 min (conditions D). HRMS (ESI⁺): m/z [M+2H]²⁺ calcd for C₉₁H₁₄₃N₁₇O₄₇S₂ 1144.9376; found 1144.9365. HRMS (ESI⁺): m/z [M+3H]³⁺ calcd for C₉₁H₁₄₄N₁₇O₄₇S₂ 763.6275; found 763.6263. HRMS (ESI⁺): m/z [M+H+Na]²⁺ calcd for C₉₁H₁₄₂N₁₇O₄₇S₂Na, 1155.9268; found 1155.9278.

Conversion of Decasaccharide 89b into an (AB)₅-TT Conjugate.

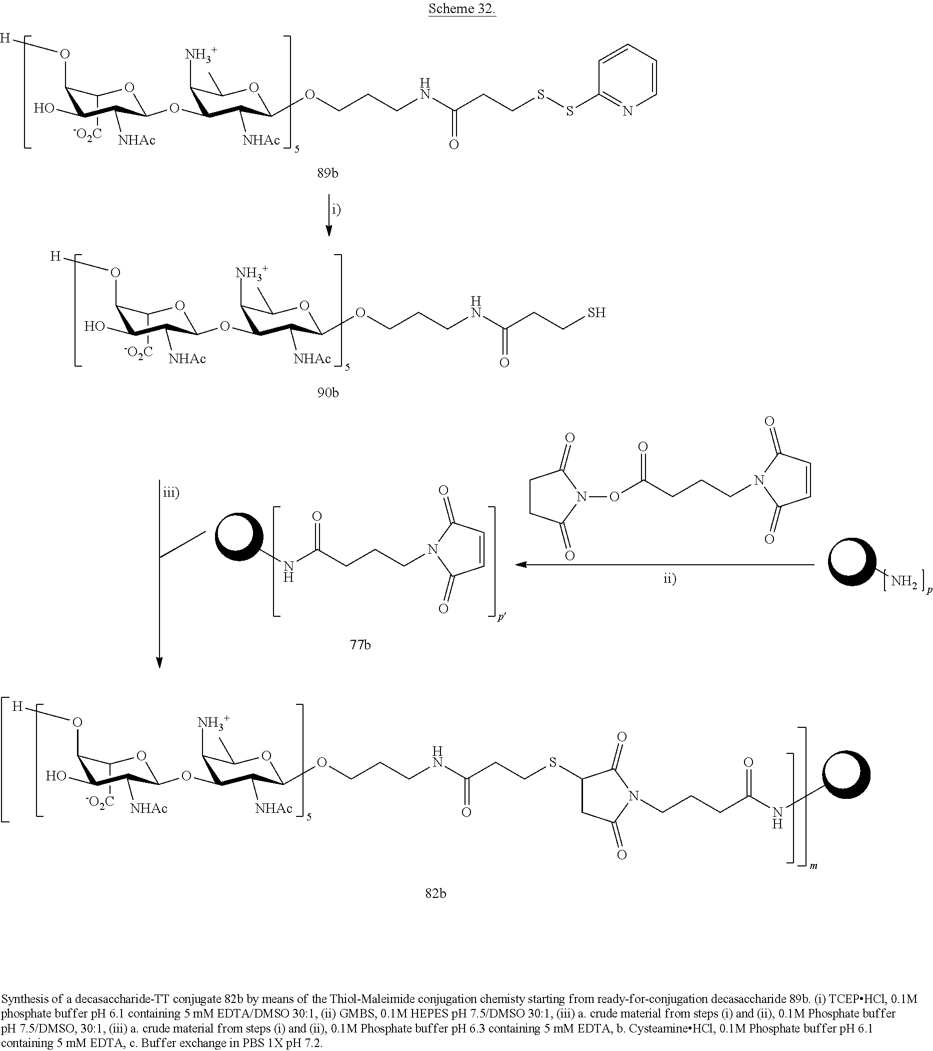

Decasaccharide-TT conjugates (82b) from thiol-equipped decasaccharide 90b. SEC-purified tetanus toxoid (TT, 150 kD, 389 μL, 6.17 mg/mL) was buffer exchanged with 0.1 M HEPES pH 7.5 by passing through a 30 kD centrifugal filter four times and finally concentrated to 230 μL (final concentration of TT: 8.77 mg/mL). A solution of GMBS (599 μg, 2.13 μmol, 160 equiv.) in DMSO (30 μL) was added to the obtained solution of TT (58 μM, 13 nmol). Modification was performed at ambient temperature for 1 h. After this time, the entire volume of intermediate 77b was buffer exchanged with 0.1 M Phosphate buffer pH 6.3 containing 5 mM EDTA, and finally concentrated to 280 μL to reach a final concentration of intermediate 77b of 7.04 mg/mL.

Decasaccharide 89b (900 μg, 393 nmol) was dissolved in 0.1 M phosphate buffer pH 6.1 containing 5 mM EDTA (150 μL). A 77 mM solution of tris(2-carboxyethyl)phosphine hydrochloride (TCEP·HCl) in DMSO (5.4 μL, 118 μg, 410 nmol, 1.04 equiv.) was added to reach a concentration of 2.6 mM of oligosaccharide and the solution was stirred at rt. After 1 h, monitoring by RP-HPLC (conditions D) and HRMS revealing the absence of the starting 89b (Rₜ=11.2 min) and the presence of a major product corresponding to the expected thiol 90b. The later had RP-HPLC (215 nm): Rₜ=9.4 min (conditions D).

The obtained solutions of intermediates 77b (TT, 280 μL, 1.97 mg, 13.1 nmol) and crude 90b (from decasaccharide 89b, 393 nmol) were mixed and gently stirred for 4 h at rt. A solution of Cysteamine·HCl (0.4 mg, 20 mg/mL in deionised water (20 μL)) was added, facilitating a molar excess of 160 compared to intermediate 77b. After stirring for 30 min at rt, the entire volume of conjugate was buffer exchanged with PBS 1× pH 7.2 and adjusted to a volume of 800 μL. The obtained conjugate 82b was analyzed by UV (λ=280 nm) and MALDI-MS for conjugation yield and carbohydrate:protein ratio.

In addition to the above TT-conjugates featuring an (AB)ₙ hapten (n=1-5, n being the number of AB repeat per chain), it is noted that TT-conjugates featuring a B(AB)ₙ hapten ({B[AB]ₙ}m-TT, Table 2, m being the number of oligosaccharide chains per TT) have been successfully synthesized using the same procedure. To that end, the ready-for-conjugation oligosaccharides bearing a masked thiol at their reducing end were obtained. They were then conjugated analogously as described above.

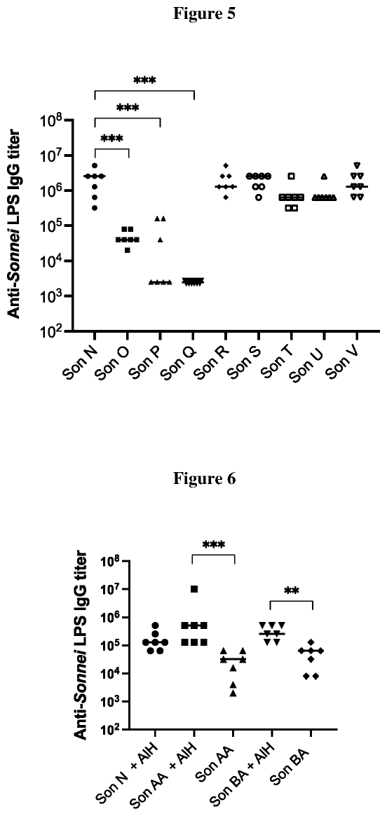

TABLE 2
S. sonnei B(AB)ₙ oligosaccharide-TT conjugates obtained by attachement at their
non reducing end ({B[AB]ₙ}ₘ-TT) or at their non-reducing end (TT-{B[AB]ₙ}ₘ).

| | | | Average | | | |
|---|---|---|---|---|---|---|
| | Code in | Conjugate | OS:TT ratio | TT conc | OS conc | Total |
| | FIG. 5 | Nb | (m) | mg/mL | μg/mL | volume |
| Ssonnei | Son O | 37c1 | 6 | 2.11 | 48 | 905 μL |
| SS-TT3 | Son P | 37c2 | 13 | 2.26 | 120 | 950 μL |
| | Son Q | 37c3 | 20 | 2.25 | 185 | 860 μL |
| Ssonnei | Son R | 38c1 | 7 | 2.15 | 100 | 975 μL |
| SS-TT5 | Son S | 38c2 | 13 | 1.85 | 156 | 910 μL |
| | Son T | 38c3 | 17 | 1.18 | 138 | 910 μL |
| Ssonnei | Son U | 39c1 | 8 | 1.74 | 130 | 1520 μL |
| SS-TT7 | | 39c2 | | | | |
| | Son V | 39c3 | 14 | 0.73 | 96 | 970 μL |
| | | 39c4 | 14 | 0.43 | 56 | 980 μL |
| | | 39c5 | 16 | 0.43 | 65 | 980 μL |
| Ssonnei | | 40c1 | 11 | 2.24 | 172 | 800 μL |
| SS-TT5_{EC(End Chain)} | | 40c2 | 16 | 1.88 | 210 | 800 μL |

### Example 18: Synthesis of B(AB)ₙ Oligosaccharides of the Invention in the Form of Azidopropyl Glycosides—Post-Chain Elongation Linker Introduction

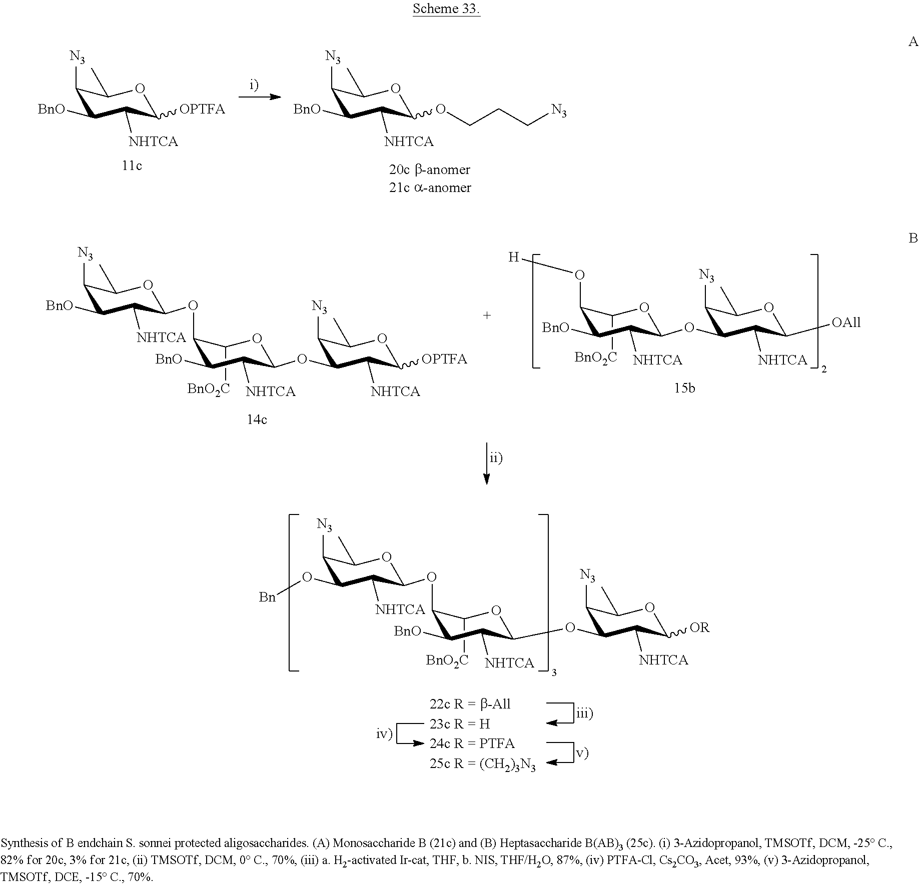

3-Azidopropyl 4-azido-2-trichloroacetamido-2,4,6-trideoxy-β-D-galactopyranoside (20c). A suspension of PTFA donor 11c (2.0 g, 3.37 mmol, 1.0 equiv.) in DCM (33 mL) containing freshly activated MS 4 Å (1.0 g) was stirred for 1 h at rt under an argon atmosphere. After cooling to −25° C. for 10 min, TMSOTf (61 μL, 337 μmol, 0.1 equiv.) was added slowly. After stirring for 1 h at this temperature, a TLC analysis (Tol/EtOAc, 4:1) showed reaction completion. Et₃N (1.0 equiv. vs TMSOTf) was added and the heterogenous mixture was filtered. Solids were washed with DCM (2×10 mL). The combined filtrates were concentrated and the crude was purified by flash chromatography (Tol/EtOAc 85:15→80:20) to give by order of elution the α-isomer 21c (58 mg, 99 μmol, 3%) and the desired β-isomer 20c (1.6 g, 3.16 mmol, 82%) and, both as a white solid. The β-isomer 20c had R_{f} 0.45 (Tol/EtOAc 4:1). ¹H NMR (CDCl₃) δ 7.40-7.31 (m, 5H, H_{Ar}), 6.95 (d, 1H, J_{2,NH}=6.0 Hz, NH), 4.91 (d, 1H, J_{1,2}=8.4 Hz, H-1), 4.71 (d, 1H, J=11.2 Hz, CH_{2Bn}), 4.62 (d, 1H, CH_{2Bn}), 4.45 (dd, 1H, J_{2,3}=10.9 Hz, J_{3,4}=3.2 Hz, H-3), 3.96-3.91 (mₚₒ, 1H, OCH₂), 3.76 (d, 1H, H-4), 3.67 (dqₚₒ, 1H, H-5), 3.62-3.55 (mₚₒ, 2H, OCH₂, H-2), 3.38 (t, 2H, J=6.8 Hz, NCH₂), 1.90-1.78 (mₚₒ, 2H, CH₂), 1.36 (d, 3H, J_{5,6}=6.0 Hz, H-6). ¹³C NMR (CDCl₃) δ 162.0 (CO_{NHTCA}), 136.8 (C_{q,Ar}), 128.7, 128.4, 128.3 (C_{Ar}), 98.7 (C-1_{A}, ¹J_{C,H}=162 Hz), 92.4 (CCl₃), 76.0 (C-3), 72.6 (CH_{2Bn}), 69.1 (C-5), 66.4 (OCH₂), 62.9 (C-4), 55.9 (C-2), 48.1 (NCH₂), 29.0 (CH₂), 17.5 (C-6). HRMS (ESI⁺): m/z [M+NH₄]⁺ calcd for C18H₂₆Cl₃N₈O₄ 523.1137; found 523.1136.

The α-isomer 21c had R_{f} 0.5 (Tol/EtOAc 4:1). ¹H NMR (400 MHz, CDCl₃) δ 7.39-7.31 (m, 5H, H_{Ar}), 6.69 (d, 1H, J_{2,NH}=8.4 Hz, NH), 4.91 (d, 1H, J_{1,2}=3.6 Hz, H-1), 4.76 (d, 1H, J=12.0 Hz, CH_{2Bn}), 4.60 (d, 1H, CH_{2Bn}), 4.45 (dddₚₒ, 1H, H-2), 3.95 (dqₚₒ, 1H, H-5), 3.86 (ddₚₒ, 1H, J_{3,4}=3.6 Hz, H-3), 3.83 (dₚₒ, 1H, H-4), 3.80-3.74 (mₚₒ, 1H, OCH₂), 3.54-3.84 (mₚₒ, 2H, OCH₂), 3.35 (t, 2H, J=5.6 Hz, NCH₂), 1.88-1.84 (mₚₒ, 2H, CH₂), 1.32 (d, 3H, J_{5,6}=6.4 Hz, H-6). ¹³C NMR (CDCl₃) δ 161.6 (CO_{NHTCA}), 137.8 (C_{q,Ar}), 128.6, 128.2, 127.9 (C_{Ar}), 96.9 (C-1, ¹J_{C,H}=172 Hz), 92.6 (CCl₃), 76.0 (C-3), 71.7 (CH_{2Bn}), 65.3 (OCH₂), 65.1 (C-5), 62.6 (C-4), 50.0 (C-2), 48.4 (NCH₂), 28.5 (CH₂), 17.4 (C-6). HRMS (ESI⁺): m/z [M+NH₄]⁺ calcd for C₁₈H₂₆Cl₃N₈O₄ 523.1137; found 523.1136.

Allyl 4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1-4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (22c). The trisaccharide donor 14c (1.0 g, 711 μmol, 1.0 equiv.) and the tetrasaccharide acceptor 15b (1.19 g, 711 μmmol, 1.0 equiv.) were co-evaporated with anhyd. toluene (20 mL) and dried in high vacuum for 1 h. The dried mixture was dissolved in anhyd. DCM (18 mL) and stirred with freshly activated MS 4 Å (˜1.0 g) under an argon atmosphere for 1 h at rt. The reaction mixture was cooled to 0° C. and TMSOTf (7.7 μl, 43 μmol, 0.06 equiv.) was added slowly. After stirring for 1 h at 0° C., a TLC (Tol/ACN 4:1) follow up showed reaction completion. Et₃N (1.0 equiv. vs TMSOTf) was added and the reaction mixture was allowed to reach rt. The suspension was filtered over a fitted funnel, washed with DCM (3×10 mL). The filtrate was concentrated under vacuo and the crude was purified by flash chromatography (SiOH 25 μm, Tol/ACN 85:15) to give the heptasaccharide 22c as a white solid (1.46 g, 503 μmol, 70%). The desired 22c had R_{f} 0.35 (Tol/ACN 4:1). HRMS (ESI⁺): m/z [M+2NH₄]²⁺ calcd for C₁₀₈H₁₁₆C₁₂₁N₂₁O₃₁ 1470.0799; found 1470.0819.

4-Azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-α/β-D-galactopyranose (23c). [Ir(COD)(PMePh₂)₂]⁺PF₆⁻ (19 mg, 22 μmol, 0.05 equiv.) in anhyd. THF (3 mL) was stirred for 40 min under an H₂ atmosphere. The deep red color of the solution slowly turned to yellow. The mixture was degassed and transferred to a solution of heptasaccharide 22c (1.3 g, 448 mmol, 1.0 equiv.) in anhyd. THF (9 mL). After stirring overnight at rt, NIS (121 mg, 538 mmol, 1.2 equiv.) and water (4 mL) were added. After another 2 h at rt, 50% aq. Na₂S₂O₃ (5 mL) was added. Volatiles were removed under vacuum. The obtained solution was diluted with DCM (20 mL) and water (20 mL). The organic layer was separated and the aq. layer was washed with DCM (2×10). The organic parts were combined and washed with 20% aq. NaHCO₃ (10 mL) and brine (10 mL). The organic phase was dried over Na₂SO₄, concentrated. Flash chromatography (Tol/EtOAc 70:30→60:40) gave the hemiacetal 23c as a white solid (1.12 g, 391 μmol, 87%). Hemiacetal 23c had R_{f} 0.5 (Tol/EtOAc 4:1). HRMS (ESI⁺): m/z [M+2NH₄]²⁺ calcd for C₁₀₅H₁₁₂Cl₂₁N₂₁O₃₁ 1450.5620; found 1450.5630. HRMS (ESI⁺): m/z [M+2NH₄]²⁺ calcd for C₁₀₅H₁₁₂Cl₂₁N₂₁O₃₁ 1450.5620; found 1450.5630.

4-Azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-α/β-D-galactopyranosyl (N-phenyl)trifluoroacetimidate (24c). To a solution of hemiacetal 23c (1.12 g, 391 μmol, 1.0 equiv.) in acetone (15 mL) were successively added PTFA-Cl (79 μL, 500 μmol, 1.3 equiv.) and Cs₂CO₃ (150 mg, 461 μmol, 1.2 equiv.). After stirring at rt for 2 h, the suspension was filtered over a Celite bed, washed with acetone (2×5 mL). The filtrate was concentrated. Flash chromatography (cHex/EtOAc 60:40→50:50) gave the PTFA donor 24c (1.09 g, 359 μmol, 93%) as a white solid. The obtained 24c had R_{f} 0.55 (Tol/EtOAc 4:1). HRMS (ESI⁺): m/z [M+NH₄]⁺ calcd for C₁₁₃H₁₁₂Cl₂₁F₃N₂₁O₃₁ 3053.1244; found 3053.1379.

3-Azidopropyl 4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-3-O-benzyl-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranosyl-(1→4)-(benzyl 3-O-benzyl-2-deoxy-2-trichloroacetamido-α-L-altropyranosyluronate)-(1→3)-4-azido-2-trichloroacetamido-2,4,6-trideoxy-β-D-galactopyranoside (25c). To a solution of the PTFA donor 24c (1.09 g, 359 μmol, 1.0 equiv.) in anhyd. DCE (18 mL) was added 3-azidopropanol (66 μL, 719 μmol, 2.0 equiv.). The solution was stirred with freshly activated MS 4 Å (800 mg) for 1 h under an argon atmosphere and then cooled to −15° C. TMSOTf (7.0 μL, 36 μmol, 0.1 equiv., in anhyd. ACN (500 μL)) was added slowly. After stirring at −15° C. for 45 min, a TLC analysis (Tol/EtOAc 7:3) showed reaction completion. Et₃N (10 μL) was added. The suspension was filtered over a fitted funnel. Solids were washed with DCM (2×5 mL) and the filtrate was concentrated. Flash chromatography (SiOH 25 μm, Tol/ACN 85:15) gave the azidopropyl glycoside 25c as a white solid (750 mg, 254 μmol, 70%). Heptasaccharide 25c had R_{f} 0.3 (Tol/ACN 4:1). HRMS (ESI⁺): m/z [M+2NH₄]²⁺ calcd for C₁₀₈H₁₁₇Cl₂₁N₂₄O₃₁ 1492.5872; found 1492.5849. HRMS (ESI⁺): m/z [M+NH₄]⁺ calcd for C₁₀₈H₁₁₇³⁵Cl₁₇³⁷Cl₄N₂₄O₃₁ 2971.1342; found 2971.1380.

### Example 19: Synthesis of Oligosaccharides of the Invention Featuring a B Endchain Residue in the Form of Aminopropyl Glycosides and of the Corresponding Ready-for-Conjugation Oligosaccharides

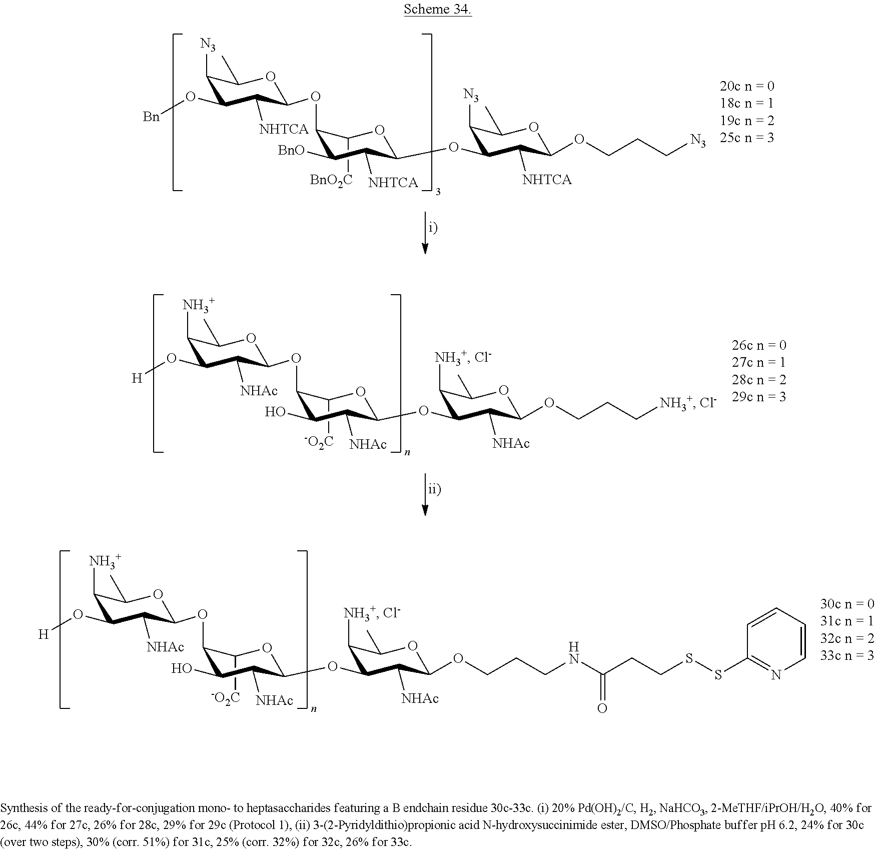

3-Aminopropyl 2-acetamido-4-amino-2,4,6-trideoxy-β-D-galactopyranoside (26c). Isopropanol (30 mL) and water (15 mL) were added to a solution of the AAT derivative 20c (50 mg, 99 μmol, 1.0 equiv.) in 2-MeTHF (2 mL). NaHCO₃ (25 mg, 1.18 mmol, 3.0 equiv.) in water (1 mL) was added to the solution followed by the addition of 20% Pd(OH)₂/C (280 mg, 396 μmol, 4.0 equiv.). The suspension was stirred under a hydrogen atmosphere for 8 h at rt, filtered over a bed of Celite®, washed with methanol (10 mL) and water (10 mL). The filtrate was concentrated and the crude product was purified by semi-preparative RP-HPLC. The free monosaccharide 26c was obtained as a white lyophilized solid (10.5 mg, 40 μmol, 40%). Diamine 26c had RP-HPLC (215 nm/ELSD) Rₜ=3.1/3.2 min (conditions A). ¹H NMR (D₂O) δ 4.48 (d, 1H, J_{1,2}=8.4 Hz, H-1), 4.09-3.97 (mₚₒ, 3H, H-3, H-5, OCH₂), 3.77-3.67 (mₚₒ, 2H, H-2, OCH₂), 3.60 (brd, 1H, J_{3,4}=4.4 hz, H-4), 3.06 (t, 2H, J=7.2 Hz, NCH₂), 2.04 (s, 3H, CH_{3Ac}), 1.96-1.90 (m, 2H, CH₂), 1.26 (d, 3H, J_{5,6}=6.8 Hz, CH₃). ¹³C NMR (D₂O) 175.1 (CO_{Ac}), 101.6 (C-1_{A}, ¹J_{C,H}=163 Hz), 68.1 (OCH₂), 67.6 (C-3), 67.5 (C-5), 54.8 (C-4), 52.1 (C-2), 37.6 (NCH₂), 26.7 (CH₂), 22.2 (CH_{3Ac}), 15.7 (C-6). HRMS (ESI⁺): m/z [M+H]⁺ calcd for C₁₁H₂₄N₃O₄ 262.1761; found 262.1763.

3-Aminopropyl (2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (27c). Isopropanol (60 mL) and Milli-Q water (12 mL) were added to a solution of the protected trisaccharide 18c (200 mg, 152 μmol, 1.0 equiv.) in 2-MeTHF (2 mL). 20% Pd(OH)₂/C (320 mg, 455 μmol, 3.0 equiv.) was added and the mixture was stirred for 20 h at rt under hydrogen. During this timeframe, NaHCO₃ (115 mg, 1.36 mmol, 9.0 equiv.) in Milli-Q water (3.0 mL) was added slowly by means of an automated syringe pump. The suspension was passed through a bed of Celite® and washed with isopropanol/water (1:1, v/v, 20 mL). Volatiles were eliminated under reduced pressure and the aqueous phase was lyophilized. Semi-preparative RP-HPLC of the residue gave the aminopropyl-equipped trisaccharide 27c as a white lyophilized solid (45 mg, 68 μmol, 44%). The free trisaccharide 27c had RP-HPLC (215 nm/ELSD) Rₜ=6.2/7.3 min (conditions A). ¹H NMR (D₂O) δ 4.76-4.74 (po, 1H, H-1_{A}), 4.70 (po, 1H, H-11), 4.43 (brs, 1H, H-5_{A}), 4.39 (d, 1H, J_{1,2}=8.4 Hz, H-1_{B}), 4.09-4.06 (ddₚₒ, 1H, H-3_{B}), 4.04-3.90 ((mₚₒ, 5H, H-3_{B1}, H-5_{B}, H-5_{B1}, H-4_{A}, OCH₂), 3.85-3.72 (mₚₒ, 5H, H-2_{B}, H-2_{B1}, H-4_{B}, H-2_{A}, H-3_{A}), 3.64-3.59 (mₚₒ, 1H, OCH₂) 3.52 (br, 1H, H-4_{B1}), 2.98 (t, 2H, J=6.4 Hz, NCH₂), 1.97 (s, 3H, CH_{3Ac}), 1.95 (s, 3H, CH_{3Ac}), 1.91 (s, 3H, CH_{3Ac}), 1.89-1.85 (mₚₒ, 2H, CH₂), 1.26 (br, 6H, CH₃). ¹³C NMR (400 MHz, D₂O) 175.4, 174.4, 174.2 (CO_{NHAc}), 171.6 (C-6_{A}), 103.0 (C-1_{B1}, ¹J_{C,H}=163 Hz), 101.6 (C-1_{B}, ¹J_{C,H}=163 Hz), 101.2 (C-1_{A}, ¹J_{C,H}=166 Hz), 76.6 (C-4_{A}), 76.1 (C-5_{A}), 75.8 (C-3_{B}), 68.2 (OCH₂), 67.6 (C-3_{A}, C-3_{B1}), 67.6 (C-5_{B}), 67.4 (C-5_{B1}), 54.8 (C-4_{B}), 54.7 (C-4_{B1}), 52.4 (C-2_{B}), 51.5 (C-2_{A}), 50.8 (C-2_{A1}), 37.6 (NCH₂), 26.7 (CH₂), 22.3 (CH_{3Ac}), 22.2 (CH₃,Ac), 15.7 (C-6_{B}), 15.6 (C-6_{B1}). HRMS (ESI⁺): m/z [M+H]⁺ calcd for C₂₇H₄₉N₆O₁₃ 665.3352; found 665.3336. HRMS (ESI⁺): m/z [M+Na]⁺ calcd for C₂₇H₄₈N₆O₁₃Na, 687.3172; found 687.3152.

3-Aminopropyl (2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (28c). To a solution of the pentasaccharide 19c (120 μmg, 56 μmol, 1.0 equiv.) in 2-MeTHF/isopropanol/water (1:15:3, v/v/v, 76 mL) were added 20% Pd(OH)₂/C (160 mg, 225 μmol, 4.0 equiv.) and aq. NaHCO₃ (400 μL, from a stock solution made of NaHCO₃ (71 mg, 845 μmol, 15 equiv.) in Milli-Q water (1.5 mL)). The suspension was stirred in a hydrogen atmosphere for 42 h, while slowly adding the remaining aq. NaHCO₃ stock solution (200 μL/2 h). At completion, the suspension was passed through a bed of Celite®, washed with isopropanol (2×10 mL) and water (2×10 mL). The filtrate was concentrated under reduced pressure and lyophilized. Semi-preparative RP-HPLC purification of the residue to give the aminopropyl glycoside 28c as a white solid (12.2 mg, 11.4 μmol, 20%). Pentasaccharide 28c had RP-HPLC (215 nm/ELSD): Rₜ=8.0/8.9 min (conditions A). HRMS (ESI⁺): m/z [M+H]⁺ calcd for C₄₃H₇₄N₉O₂₂ 1068.4943; found 1068.4946. HRMS (ESI⁺): m/z [M+2H]²⁺ calcd for C₄₃H₇₅N₉O₂₂ 534.7508; found 534.7512.

3-Aminopropyl (2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (29c). To a solution of heptasaccharide 25c (120 mg, 41 μmol, 1.0 equiv.) in 2-MeTHF/isopropanol/water (1:10:2, v/v/v) was added 20% Pd(OH)₂/C (170 mg, 245 μmol, 6.0 equiv.) and aq. NaHCO₃ (600 μL from a stock solution made of NaHCO₃ (72 mg, 856 μmol, 21.0 equiv. in water (2.1 mL)). The reaction mixture was stirred under an hydrogen atmosphere for 54 h while slowly adding the remaining aq. NaHCO₃ stock solution (200 μL/2 h). The suspension was passed through a bed of Celite, washed with isopropanol (2×10 mL) and water (2×10 mL). The filtrate was concentrated under reduced pressure and lyophilized. Purification by semi-preparative RP-HPLC gave the aminopropyl glycoside 29c as a white solid (17.8 mg, 12 μmol, 29%). Heptasaccharide 29c had RP-HPLC (215 nm/ELSD): Rₜ=7.4/7.5 min (conditions A). HRMS (ESI⁺): m/z [M+2H]²⁺ calcd for C₅₉H₁₀₀N₁₂O₃₁ 736.3303; found 736.3286. HRMS (ESI⁺): m/z [M+H+Na]²⁺ calcd for C₅₉H₉₉N₁₂O₃₁Na, 747.3213; found 747.3197.

3-(3-(2-Pyridyldithio)propionamido)-propyl 2-acetamido-4-amino-2,4,6-trideoxy-β-D-galactopyranoside (30c). Step 1: Isopropanol (45 mL) and water (22 mL) were added to a solution of the azidopropyl AAT 20c (132 mg, 297 μmol, 1.0 equiv.) in 2-MeTHF (4.0 mL). 20% Pd(OH)₂/C (835 mg, 1.18 mmol, 4.0 equiv.) was added followed by the addition of NaHCO₃ (75 mg, 891 μmol, 3.0 equiv.) in water (1.0 mL). The reaction mixture was stirred for 8 h under a hydrogen atmosphere at rt. At completion, the suspension was filtered, washed with methanol (2×15 mL) and concentrated. The crude 26c was subjected to the next step without further purification.

Step 2: SPDP linker (40 mg, 130 μmol, 0.5 equiv. in DMSO (50 μL)) was added to a solution of crude amine 26c (291 μmol theoretical, 1.0 equiv.) in 0.1 M phosphate buffer pH 6.2 (3.3 mL, 0.1 M). The resulting suspension was stirred at rt for 48 h, during which time the precipitate disappeared slowly. At completion based on RP-HPLC monitoring, the reaction mixture was passed through a 0.2 μm centrifuge filter. The filtrate was purified by semi-preparative RP-HPLC to give the desired 30c as a white lyophilized solid (29 mg, 63 μmol, 24%). The linker-equipped AAT 30c had RP-HPLC (215 nm/ELSD): Rₜ=11.0/11.0 min (conditions E). ¹H NMR (D₂O, 400 MHz) δ 8.51 (d, 1H, J=4.4 Hz, H_{Ar}), 8.12 (dtₚₒ, 1H, J=6.4 Hz, H_{Ar}), 8.01 (d, 1H, J=8.4 Hz, H_{Ar}), 7.55 (dtₚₒ, 1H, J=5.6 Hz, H_{Ar}), 4.45 (dₚₒ, 1H, J_{1,2}=8. Hz, H-1), 4.05-3.96 (mₚₒ, 2H, H-3, H-5), 3.90-3.84 (mₚₒ, 1H, OCH₂), 3.74-3.69 (mₚₒ, 1H, OCH₂), 3.61-3.55 (mₚₒ, 2H, H-2, H-4), 3.23-3.13 (mₚₒ, 2H, NCH₂), 3.09 (t, 2H, J=6.0 Hz, CH₂), 2.64 (t, 2H, J=6.8 Hz, SCH₂₋ₗᵢₙₖₑᵣ), 2.01 (s, 3H, CH_{3Ac}), 1.74-1.68 (m, 2H, COCH₂₋ₗᵢₙₖₑᵣ), 1.30 (d, 3H, J_{5,6}=6.4 Hz, H-6). ¹³C NMR (D₂O, 400 MHz) δ 174.9 (CO_{NHAc}), 173.4 (COₗᵢₙₖₑᵣ), 157.1 (C_{q,Ar}), 145.6, 142.4, 123.6, 123.1 (C_{Ar}), 101.6 (C-1), 68.0 (OCH₂), 67.7 (C-3), 67.5 (C-5), 54.8 (C-4), 52.1 (C-2), 36.2 (NCH₂), 34.5 (SCH₂), 34.1 (COCH₂), 28.3 (CH_{2Pr}), 22.2 (CH_{3Ac}), 15.6 (C-6). HRMS (ESI⁺): m/z [M+H]⁺ calcd for C₉H₃₁N₄O₅S₂ 459.1730; found 459.1723. HRMS (ESI⁺): m/z [M+Na]⁺ calcd for C₉H₃₀N₄O₅S₂Na, 481.1550; found 481.1543.

3-(3-(2-Pyridyldithio)propionamido)-propyl (2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (31c). The aminopropyl trisaccharide 27c (10 mg, 15 μmol, 1.0 equiv.) was dissolved in 0.1 M phosphate buffer pH 6.2 (1.2 mL). A solution of SPDP (4.7 mg, 15 μmol, 1.0 equiv.) in DMSO (20 μL) was added slowly. The reaction mixture was stirred at rt for 48 h. At completion based on RP-HPLC monitoring, the crude reaction was passed through a 0.2 μm centrifuge filter. The filtrate was purified by semi-preparative RP-HPLC to give first the unreacted 27c (4.1 mg) and then the desired 31c, both as a white lyophilized solid (3.9 mg, 4.5 μmol, 30%, corrected yield 51%). Amine 31c had RP-HPLC (215 nm/ELSD): Rₜ=16.2/17.0 min (conditions D). ¹H NMR (D₂O, 400 MHz) δ 8.53 (d, 1H, J=5.6 Hz, H_{Ar}), 8.21 (dtₚₒ, 1H, J=7.6 Hz, H_{Ar}), 8.07 (d, 1H, J=8.4 Hz, H_{Ar}), 7.62 (dtₚₒ, 1H, J=6.0 Hz, H_{Ar}), 4.74 (dₚₒ, 1H, J_{1,2}=8.8 Hz, H-1_{A}), 4.72 (po with HOD, 1H, H-1_{B1}), 4.61 (brs, 1H, H-5_{A}), 4.423 (dₚₒ, 1H, H-4_{A}), 4.40 (d, 1H, J_{1,2}=8.6 Hz, H-1_{B}), 4.19 (dd, 1H, J_{2,3}=11.0 Hz, J_{3,4}=4.0 Hz, H-331), 4.05-3.97 (mₚₒ, 3H, H-31, H-5_{B}, H-531), 3.86-3.78 (mₚₒ, 3H, H-2_{A}, H-231, OCH₂), 3.77-3.69 (mₚₒ, 3H, H-21, H-3_{A}, H-431), 3.57-3.51 (mₚₒ, 2H, H-41, OCH₂), 3.16-3.07 (mₚₒ, 2H, NCH₂), 3.06 (tₚₒ, 2H, J=6.4 Hz, COCH₂-linker), 2.61 (t, 2H, J=6.4 Hz, SCH₂₋ₗᵢₙₖₑᵣ), 1.98, 1.97, 1.92 (3s, 9H, CH_{3Ac}), 1.70-1.63 (m, 2H, CH₂), 1.29 (dₚₒ, 3H, J_{5,6}=6.8 Hz, H-6_{B1*}), 1.26 (dₚₒ, 3H, J_{5,6}=6.8 Hz, H-6_{B*}). ¹³C NMR (D₂O, 400 MHz) δ 175.4, 174.5, 174.0 (CO_{NHAc}), 173.4 (COₗᵢₙₖₑᵣ), 172.7 (C-6_{A}), 156.7 (C_{q,Ar}), 144.5, 143.7, 124.5, 123.6 (C_{Ar}), 103.0 (C-1_{B1}, ¹J_{C,H}=165 Hz), 101.6 (C-1_{B}, ¹J_{C,H}=162 Hz), 101.1 (C-1_{α}, ¹J_{C,H}=165 Hz), 77.0 (C-4_{A}), 76.6 (C-5_{A}), 76.0 (C-3_{B1}), 68.1 (OCH₂), 67.8 (C-3_{B}), 67.6 (C-5_{B}), 67.6 (C-5_{B1}), 67.4 (C-3_{A}), 54.6 (C-4_{B}, C-4_{B1}), 52.4 (C-2_{B}), 51.6 (C-2_{A}), 50.9 (C-2_{B1}), 36.3 (NCH₂), 34.5 (SCH₂), 34.3 (COCH₂), 28.4 (CH_{2Pr}), 22.3, 22.3 (2C, CH_{3Ac}), 15.7, 15.6 (C-6_{B}, C-6_{B1}). HRMS (ESI⁺): m/z [M+H]⁺ calcd for C₃₅H₅₅N₇O₁₄S₂ 862.3321; found 862.3300. HRMS (ESI⁺): m/z [M+Na]⁺ calcd for C₃₅H₅₅N₇O₁₄S₂Na, 884.3141; found 884.3118.

3-(3-(2-Pyridyldithio)propionamido)-propyl (2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (32c). To a solution of the aminopropyl pentasaccharide 28c (7.1 mg, 6.7 μmol, 1.0 equiv.) was dissolved in 0.1 M phosphate buffer pH 6.2 (1.6 mL). A solution of SPDP (2.0 mg, 6.7 μmol, 1.0 equiv.) in DMSO (50 μL) was added slowly while the reaction mixture was stirred at rt. After 8 h, more SPDP (311 μg, 0.15 equiv.) in DMSO (25 μL) was added. The reaction run for another 16 h and the suspension was passed through a 0.2 μm centrifuge filter. The filtrate was purified by semi-preparative RP-HPLC to give first the unreacted 28c (1.6 mg) followed by the expected 32c (2.1 mg, 1.6 μmol, 25%, corrected yield 32%), both as a white solid. The ready-for-conjugation pentasaccharide 32c had RP-HPLC (215 nm/ELSD): Rₜ=11.4/11.5 min (conditions D). HRMS (ESI⁺): m/z [M+2H]²⁺ calcd for C₅₁H₈₂N₁₀O₂₃S₂ 633.2492; found 633.2480.

3-(3-(2-Pyridyldithio)propionamido)-propyl (2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (33c). To a solution of the aminopropyl heptasaccharide 29c (6.0 mg, 4.1 μmol, 1.0 equiv.) in 0.1 M phosphate buffer pH 6.2 (1.0 mL). SPDP (1.3 mg, 4.1 μmol, 1.0 equiv.) in DMSO (30 μL) was added After 20 h at rt, a LCMS analysis indicated the presence of the desired product. The suspension was filtered by passing through a 0.2 μm centrifuge filter. The filtrate was purified by semi-preparative RP-HPLC to give 33c (1.8 mg, 1.42 μmol, 26%) as a white lyophilized solid. The ready-for-conjugation heptasaccharide 33c had RP-HPLC (215 nm/ELSD): Rₜ=15.5/16.4 min (conditions D). HRMS (ESI⁺): m/z [M+2H]²⁺ calcd for C₆₇H₁₀₇N₁₃O₃₂S₂ 834.8266; found 834.8288. HRMS (ESI⁺): m/z [M+H+Na]²⁺ calcd for C₆₇H₁₀₆N₁₃O₃₂S₂Na, 845.8197; found 845.8171.

### Example 20: Synthesis of Oligosaccharide-TT Conjugates of the Invention from Oligosaccharides Precursors Comprising a B Endchain Residue and a Masked Thiol Linker at their Reducing End

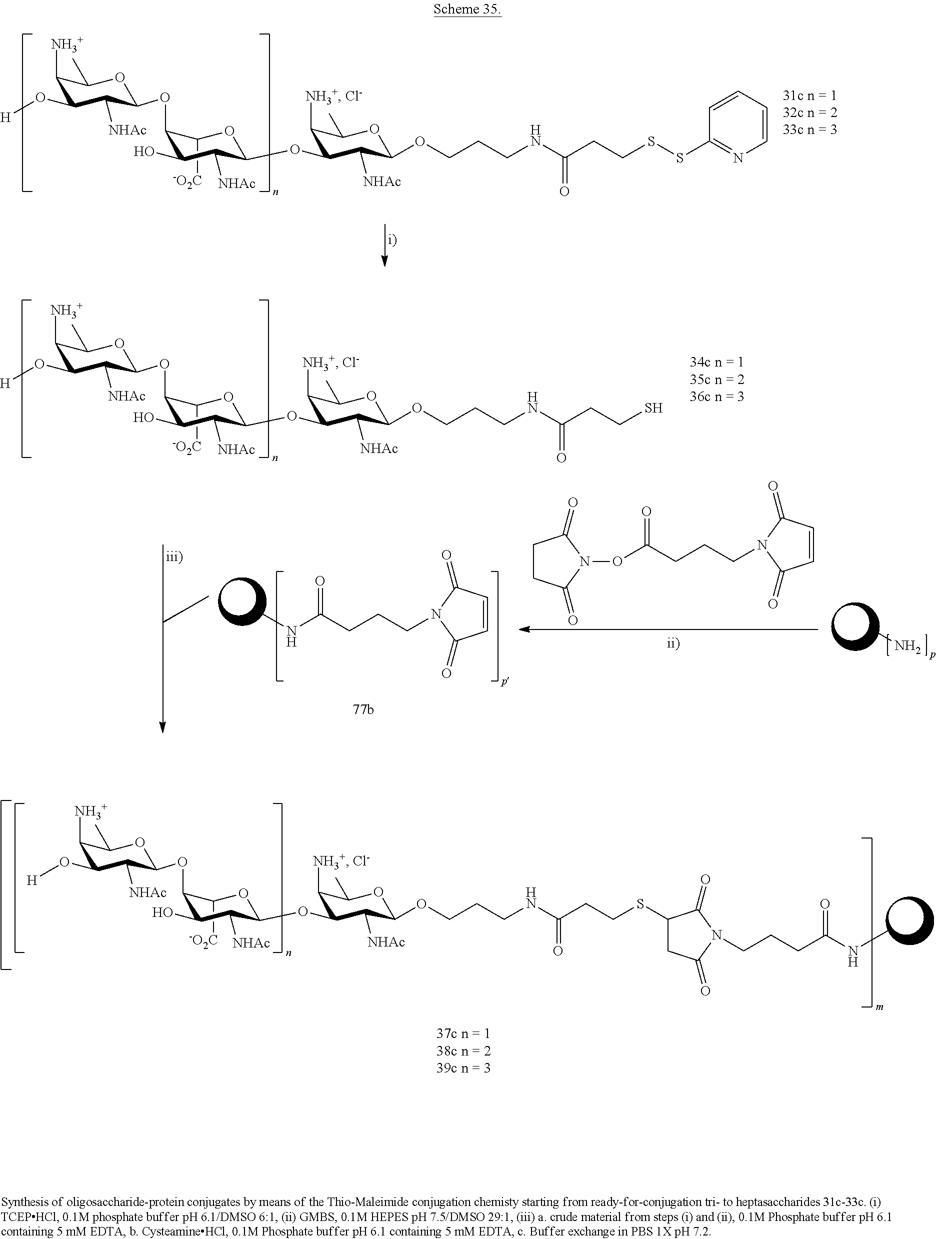

Trisaccharide-TT conjugates (37c) from thiol-equipped trisaccharide 34c. SEC purified tetanus toxoid (TT, 150 kD, 1.0 mL, 6.16 mg/mL) was buffer exchanged with 0.1 M HEPES pH 7.5 by passing through a 30kD centrifugal filter four times and finally concentrated to 400 μL (final concentration of TT: 15.6 mg/mL). A solution of GMBS (1.85 mg, 6.6 μmol, 160 equiv.) in DMSO (20 μL) was added in two portions to the obtained solution of TT (104 μM, 41 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 410 μL to reach a final concentration of intermediate 77b of 15.1 mg/mL.

Trisaccharide 31c (300 μg, 348 nmol) was dissolved in 0.1 M phosphate buffer pH 6.1 containing 5 mM EDTA (150 μL). A 67 mM solution of TCEP-HCl in DMSO (5.2 μL, 100 μg, 349 nmol, 1.0 equiv.) was added and the solution was stirred at rt for 1 h. Monitoring was achieved by RP-HPLC (conditions D) and HRMS revealing the absence of the starting 31c (Rₜ=15.7 min) and the presence of a major product corresponding to the expected thiol 34c. The later had RP-HPLC (215 nm/ELSD): Rₜ=11.6/12.4 min (conditions D). Thiol 34c had LC-MS (ESI⁺): m/z [M]⁺ calcd for C₃₀H₅₂N₆O₁₄S, 753.3; found 753.2.

A portion of the obtained solution of crude 34c (138 μL, 318 μmol theo, 24 equiv.) was mixed with a portion of the stock solution of modified TT (77b, 132 μL, 2.0 mg, 13.3 nmol) and gently stirred for 3 h at rt. A solution of Cysteamine·HCl (0.4 mg, 20 mg/mL in deionised water (20 μL)) was added, facilitating a molar excess of 160 compared to intermediate 77b. After stirring for 30 min at rt, the entire volume of conjugate was buffer exchanged with PBS 1× pH 7.2 and finally harvested. The final conjugate 37c1 was analyzed by UV (λ=280 nm) and MALDI-MS for conjugation yield and carbohydrate:protein ratio.

In another experiment, trisaccharide 31c (1.96 mg, 2.27 μmol) was dissolved in 0.1 M phosphate buffer pH 6.1 containing 5 mM EDTA (200 μL). A 67 μM solution of TCEP-HCl in DMSO (34 μL, 653 μg, 2.28 μmol, 1.0 equiv.) was added and the solution was stirred at rt for 1 h. Monitoring was achieved by RP-HPLC (conditions D) as above to give the expected thiol 34c.

Different individual portions of various stock solutions of intermediates 77b and the obtained 34c, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. The final conjugates were analyzed by UV (λ=280 nm) and MALDI-MS for conjugation yield and carbohydrate:protein ratio.

Conjugate 37c2: From the stock solution of modified TT (77b, 132 μL, 2.0 mg, 13.3 nmol) and crude thiol 34c (60 μL, 580 nmol, 44 equiv.).

Conjugate 37c3: From the stock solution of modified TT (77b, 132 μL, 2.0 mg, 13.3 nmol) and crude thiol 34c (95 μL, 930 nmol, 70 equiv.).

Pentasaccharide-TT conjugates (38c) from thiol-equipped pentasaccharide 35c. SEC purified tetanus toxoid (TT, 150 kD, 1.95 mL, 5.76 mg/mL) was buffer exchanged with 0.1 M HEPES pH 7.5 by passing through a 30kD centrifugal filter four times and finally concentrated to 760 μL (final concentration of TT: 14.7 mg/mL). A solution of GMBS (3.3 mg, 11.8 μmol, 160 equiv.) in DMSO (20 μL) was added in two portions to the obtained solution of TT (97 μM, 74 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 650 μL to reach a final concentration of intermediate of 17.15 mg/mL.

Pentasaccharide 32c (1.8 mg, 1.4 μmol) was dissolved in 0.1 M phosphate buffer pH 6.1 (150 μL). A 63 mM solution of TCEP-HCl in 0.1 M phosphate buffer pH 6.1 (23 μL, 410 μg, 1.4 μmol, 1.0 equiv.) was added and the solution was stirred at rt for 1 h. Monitoring was achieved by RP-HPLC (conditions D) and HRMS revealing the absence of the starting 32c (Rₜ=11.2/11.4 min) and the presence of a major product corresponding to the expected thiol 35c. The later had RP-HPLC (215 nm): Rₜ=8.8/8.9 min (conditions D). Thiol 35c had HRMS (ESI⁺): m/z [M+2H]²⁺ calcd for C₄₆H₇₉N₉O₂₃S m/z 578.7499; found 578.7491.

Different individual portions of various stock solutions of intermediates 77b and 35c, respectively, were used to obtain conjugates with different carbohydrate:protein ratios. The expected amounts of each one of the two solutions were mixed and gently stirred for 3 h at rt. Cysteamine·HCl (0.4 mg, 3.0 μmol, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. The final conjugates were analyzed by UV (λ=280 nm) and MALDI-MS for conjugation yield and carbohydrate:protein ratio.

Conjugate 38c1: From the stock solution of modified TT (77b, 115 μL, 2.0 mg, 13.3 nmol) and crude thiol 35c (38.7 μL, 319 nmol, 24 equiv.).

Conjugate 38c2: From the stock solution of modified TT (77b, 105 μL, 1.8 mg, 12.0 nmol) and crude thiol 35c (64 μL, 526 nmol, 44 equiv.).

Conjugate 38c3: From the stock solution of modified TT (77b, 69 μL, 1.2 mg, 8.0 nmol) and crude thiol 35c (67 μL, 550 nmol, 70 equiv.).

Heptasaccharide-TT conjugates (39c) from thiol-equipped heptasaccharide 36c. SEC purified tetanus toxoid (TT, 150 kD, 730 μL, 6.15 mg/mL) was buffer exchanged with 0.1 M HEPES pH 7.5 by passing through a 30kD centrifugal filter four times and finally concentrated to 310 μL (final concentration of TT: 14.4 mg/mL). A solution of GMBS (1.3 mg, 4.7 μmol, 160 equiv.) in DMSO (10 μL) was added to the obtained solution of TT (96 μM, 30 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 310 μL to reach a final concentration of intermediate 77b of 14.3 mg/mL.

Heptasaccharide 33c (1.0 mg, 600 nmol) was dissolved in 0.1 M phosphate buffer pH 6.1 containing 5 mM EDTA (150 μL). A 58 mM solution of TCEP·HCl in 0.1 M phosphate buffer pH 6.1 (10.3 μL, 172 μg, 600 nmol, 1.0 equiv.) was added and the solution was stirred at rt for 1 h. Monitoring was achieved by RP-HPLC (conditions D) and HRMS revealing the absence of the starting 33c (Rₜ=15.5/16.5 min) and the presence of a major product corresponding to the expected thiol 36c. The later had RP-HPLC (215 nm/ELSD): Rₜ=11.8/12.6 min (conditions D). Thiol 36c had LC-MS (ESI⁺): m/z [M+2H]²⁺ calcd for C₆₂H₁₀₅N₁₂O₃₂S, 780.8; found 780.5.

Different individual portions of various stock solutions of intermediates 77b and 36c, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested.

Conjugate 39c1: From the stock solution of modified TT (77b, 112 μL, 1.6 mg, 10.6 nmol) and crude thiol 36c (68 μL, 255 nmol, 24 equiv.).

Conjugate 39c2: From the stock solution of modified TT (77b, 77 μL, 1.1 mg, 7.3 nmol) and crude thiol 36c (86 μL, 322 nmol, 44 equiv.).

The two preparations were combined and the final conjugate was analyzed by UV (λ=280 nm) and MALDI-MS for conjugation yield and carbohydrate:protein ratio.

In another experiment, SEC purified tetanus toxoid (TT, 150 kD, 1.0 mL, 6.17 mg/mL) was buffer exchanged with 0.1 M HEPES pH 7.5 by passing through a 30kD centrifugal filter four times and finally concentrated to 420 μL (final concentration of TT: 14.7 mg/mL). A solution of GMBS (1.8 mg, 6.5 μmol, 160 equiv.) in DMSO (20 μL) was added in two portions to the obtained solution of TT (98 μM, 76 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 450 μL to reach a final concentration of intermediate 77b of 14.6 mg/mL.

Heptasaccharide 33c (1.7 mg, 1.0 μmol) was dissolved in 0.1 M phosphate buffer pH 6.1 containing 5 mM EDTA (150 μL). A 81 mM solution of TCEP-HCl in 0.1 M phosphate buffer pH 6.1 (12.5 μL, 292 μg, 1.0 μmol, 1.0 equiv.) was added and the solution was stirred at rt for 1 h. Monitoring was achieved by RP-HPLC (conditions D). Analytical data were as above.

Different individual portions of various stock solutions of intermediates 77b and 36c, respectively, were used to obtain conjugates with different carbohydrate:protein ratios. The expected amounts of each one of the two solutions were mixed and gently stirred for 3 h at rt. A solution of Cysteamine·HCl (0.4 mg, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. The final conjugates were analyzed by UV (λ=280 nm) and MALDI-MS for conjugation yield and carbohydrate:protein ratio.

Conjugate 39c3: From the stock solution of modified TT (77b, 46 μL, 675 μg, 4.5 nmol) and crude thiol 36c (69.9 μL, 440 nmol, 98 equiv.).

Conjugate 39c4: From the stock solution of modified TT (77b, 31 μL, 453 μg, 3.0 nmol) and crude thiol 36c (25.4 μL, 160 nmol, 53 equiv.).

Conjugate 39c5: From the stock solution of modified TT (77b, 31 μL, 453 μg, 3.0 nmol) and crude thiol 36c (74.1 μL, 467 nmol, 155 equiv.).

Interestingly, single-site conjugation of the oligosaccharides at their non-reducing end was also successfully achieved to yield other conjugates of the invention, for example TT-B(AB)₂ pentasaccharide conjugates, using analogous procedures, albeit starting from oligosaccharides precursors comprising a B endchain residue and a masked thiol linker at their non-reducing end.

### Example 21: Synthesis of B(AB)ₙ Oligosaccharides in the Form of Propyl Glycosides and Conversion into Ready-for-Conjugation Oligosaccharides Featuring a Masked Thiol Linker at their Non-Reducing End

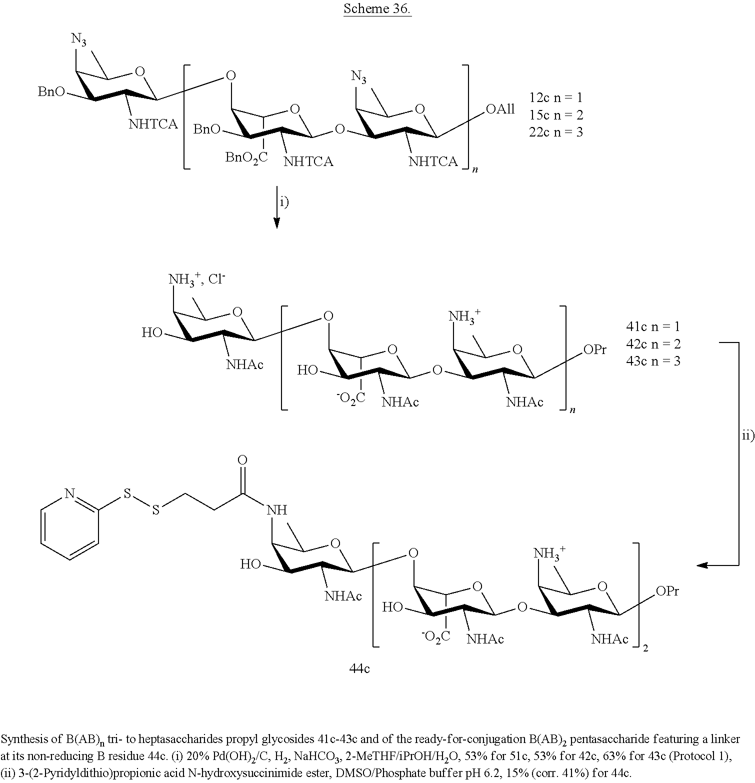

Propyl (2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (41c).^{[1]} Isopropanol (90 mL) and water (30 mL) were added to a solution of the protected trisaccharide 12c (150 mg, 118 μmol, 1.0 equiv.) in 2-MeTHF (4 mL). A solution of NaHCO₃ (89 mg, 1.05 mmol, 9.0 equiv.) in water (1.0 mL) was added followed by the addition of Pd(OH)₂/C (495 mg, 706 μmol, 6.0 equiv.). The reaction mixture stirred under a hydrogen atmosphere for 5 h at rt, at which time a LCMS follow up revealed the presence of the desired product. The suspension was passed through a bed of Celite®. Solids were washed with isopropanol (2×10 mL) and water (2×10 mL). The filtrate was concentrated under reduced pressure and the residual volume was lyophilized. Semi-preparative RP-HPLC of the crude residue gave the propyl glycoside 41c as a white lyophilized solid (41 mg, 63 μmol, 53%). Trisaccharide 41c had RP-HPLC (215 nm/ELSD): Rₜ=13.3/13.4 min (conditions A). ¹H NMR (D₂O) δ 4.78 (dₚₒ, 1H, J_{1,2}=8.8 Hz, H-1_{A}), 4.74 (dₚₒ, 1H, J_{1,2}=8.4 Hz, H-1_{B1}), 4.73 (bsₚₒ, 1H, H-5_{A}), 4.47 (brs, 1H, H-4_{A}), 4.46 (dₚₒ, 1H, J_{1,2}=8.8 Hz, H-1_{B}), 4.11 (ddₚₒ, 1H, J_{2,3}=11.2 Hz, J_{3,4}=4.0 Hz, H-3_{B1}), 4.06 (ddₚₒ, 1H, J_{2,3}=11.2 Hz, J_{3,4}=4.8 Hz, H-3_{B}), 4.05-3.98 (mₚₒ, 1H, H-5_{B}, H-5_{B1}), 3.88-3.74 (mₚₒ, 6H, H-2_{A}, H-3_{A}, H-2_{B}, H-2_{B1}, H-4_{B}, OCH_{2Pr}), 3.56 (d, 1H, H-4_{B1}), 3.54-3.48 (mₚₒ, 1H, OCH_{2Pr}), 2.00, 1.96 (2s, 9H, CH_{3Ac}), 1.53-1.46 (mₚₒ, 2H, CH_{2Pr}), 1.31 (dₚₒ, 3H, J_{5,6}=6.8 Hz, H-6_{B}), 1.30 (dₚₒ, 3H, J_{5,6}=6.9 Hz, H-6_{B1}), 0.82 (t, 3H, J=7.2 Hz, CH_{3Pr}). ¹³C NMR (D₂O) δ 175.4, 174.5, 174.0 (CO_{NHAc}, C-6_{A}), 103.0 (C-1_{B1}, ¹J_{C,H}=165 Hz), 101.6 (C-1_{B}, ¹J_{C,H}=162 Hz), 101.1 (C-1_{α}, ¹J_{C,H}=167 Hz), 76.7 (C-4_{A}), 76.1 (3C, C-3_{B}, C-3_{B1}, C-5a), 72.7 (OCH_{2Pr}), 67.7, 67.5 (C-5_{B}, C-5_{B1}), 67.4 (C-3_{A}), 54.8 (2C, C-4_{B}, C-4_{B1}), 52.3 (C-2_{B1}), 51.5 (C-2_{A}), 50.9 (C-2_{B}), 22.3, 22.2 (3C, CH_{3Ac}), 22.1 (CH_{2Pr}), 15.6, 15.5 (C-6_{B}, C-6_{B1}), 9.5 (CH_{3Pr}). HRMS (ESI⁺): m/z [M+H]⁺ calcd for C₂₇H₄₈N₅O₁₃ m/z 650.3243; found 650.3238. HRMS (ESI⁺): m/z [M+Na]⁺ calcd for C₂₇H₄₇N₅O₁₃Na m/z 672.3063; found 672.3057.

Propyl (2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (42c). Isopropanol (75 mL) and water (25 mL) to a solution of the protected pentasaccharide 15c (100 mg, 48 μmol, 1.0 equiv.) in 2-MeTHF (4 mL). A solution of NaHCO₃ (56 mg, 718 μmol, 14.0 equiv.) in water (1.4 mL) was added followed by the addition of 20% Pd(OH)₂/C (505 mg, 670 μmol, 15.0 equiv.). The reaction mixture was stirred under hydrogen for 24 h at which time solid NaHCO₃ (4.0 mg, 1.0 equiv. NaHCO₃ was added. The suspension was passed through a bed of Celite and solids were washed with isopropanol (2×10 mL) and water (2×10 mL). Volatiles were removed under reduced pressure and remaining solution filtered through a 0.2 μm syringe filter. The filtrated was lyophilized and the residue was purified by semi-preparative RP-HPLC to give the propyl glycoside 42c as a white lyophilized solid (27 mg, 26 μmol, 53%). Pentasaccharide 42c had RP-HPLC (215 nm/ELSD): Rₜ=13.4/13.5 min (conditions A). HRMS (ESI⁺): m/z [M+2H]²⁺ calcd for C₄₃H₇₄N₈O₂₂ m/z 527.2453; found 527.2440. HRMS (ESI⁺): m/z [M+Na]⁺ calcd for C₄₃H₇₂N₈O₂₂Na m/z 1075.4653; found 1075.4628.

Propyl (2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (43c). Isopropanol (90 mL) and water (30 mL) were added to a solution of the fully protected heptasaccharide 22c (120 mg, 41 μmol, 1.0 equiv.) in 2-MeTHF (4 mL). Solid NaHCO₃ (73 mg, 869 μmol, 21.0 equiv.) was added followed by 20% Pd(OH)₂/C (610 mg, 869 μmol, 21.0 equiv.). The reaction mixture was stirred for 24 h under a hydrogen atmosphere at rt. At this time, a LCMS analysis showed that the desired compound was the major product. The suspension was filtered over a bed of Celite®. The residue was washed with isopropanol (20 mL) and water (20 mL). The combined filtrate was concentrated (50 mL) and the remaining aqueous solution was filtered with 0.2 μm syringe filter. The filtrate was lyophilized and the crude was purified by semi-preparative RP-HPLC to give the propyl glycoside 43c as a white lyophilized solid (39 mg, 27 μmol, 64%). Heptasaccharide 43c had RP-HPLC (215 nm): Rₜ=14.0 min (conditions A). HRMS (ESI⁺): m/z [M+2H]²⁺ calcd for C₅₉H₉₇N₁₁O₃₁ m/z 728.8249; found 728.8234. HRMS (ESI⁺): m/z [M+H+Na]²⁺ calcd for C₅₉H₉₈N₁₁O₃₁Na m/z 739.8158; found 739.8142.

Propyl (2-acetamido-4-(3-(2-pyridyldithio)propionamido)-2,4,6-trideoxy-β-D-galactopyranosyl)-(1→4)-(2-acetamido-2-deoxy-α-L-altropyranosyluronic acid)-(1→3)-(2-acetamido-4-amino-2,4,6-trideoxy-β-D-galactopyranosyl)-(1→4)-(2-acetamido-2-deoxy-α-L-altropyranosyluronic acid)-(1→3)-2-acetamido-4-amino-2,4,6-trideoxy-β-D-galactopyranoside (44c). SPDP (14.2 mg, 46 μmol, 4.0 equiv.) in DMSO (40 μL) was added to a solution of pentasaccharide 42c (12.0 mg, 11.4 μmol, 1.0 equiv.) in 0.2 M phosphate buffer pH 6.6 (1.2 mL). 0.2 M Phosphate buffer pH 6.6 (0.5 mL) was added. After another 16 h, the suspension had turned into a clear solution. At 36 h, more SPDP (7.1 mg, 23 μmol, 2.0 equiv.) in DMSO (20 μL) was added. 24 h later, the reaction mixture was filtered using a 0.2 μm centrifuge tube. The filtrate was purified by semi-preparative RP-HPLC to give firstly the unreacted 44c (8.0 mg) and then the desired pentasaccharide 44c (2.1 mg, 1.66 μmol, 15%, corr. 44%), both as a white lyophilized solid. The linker-equipped 37c had RP-HPLC (215 nm/ELSD): Rₜ=12.0/12.1 min (conditions D). HRMS (ESI⁺): m/z [M+2H]²⁺ calcd for C₅₁H₈₁N₉O₂₃S₂ m/z 625.7438; found 625.7428. HRMS (ESI⁺): m/z [M+H]⁺ calcd for C₅₁H₈₀N₉O₂₃S₂ m/z 1250.4803; found 1250.4785.

### Example 22: Synthesis of Oligosaccharide-TT Conjugates of the Invention from Oligosaccharides Precursors Comprising a B Endchain Residue and a Masked Thiol Linker at their Non-Reducing End

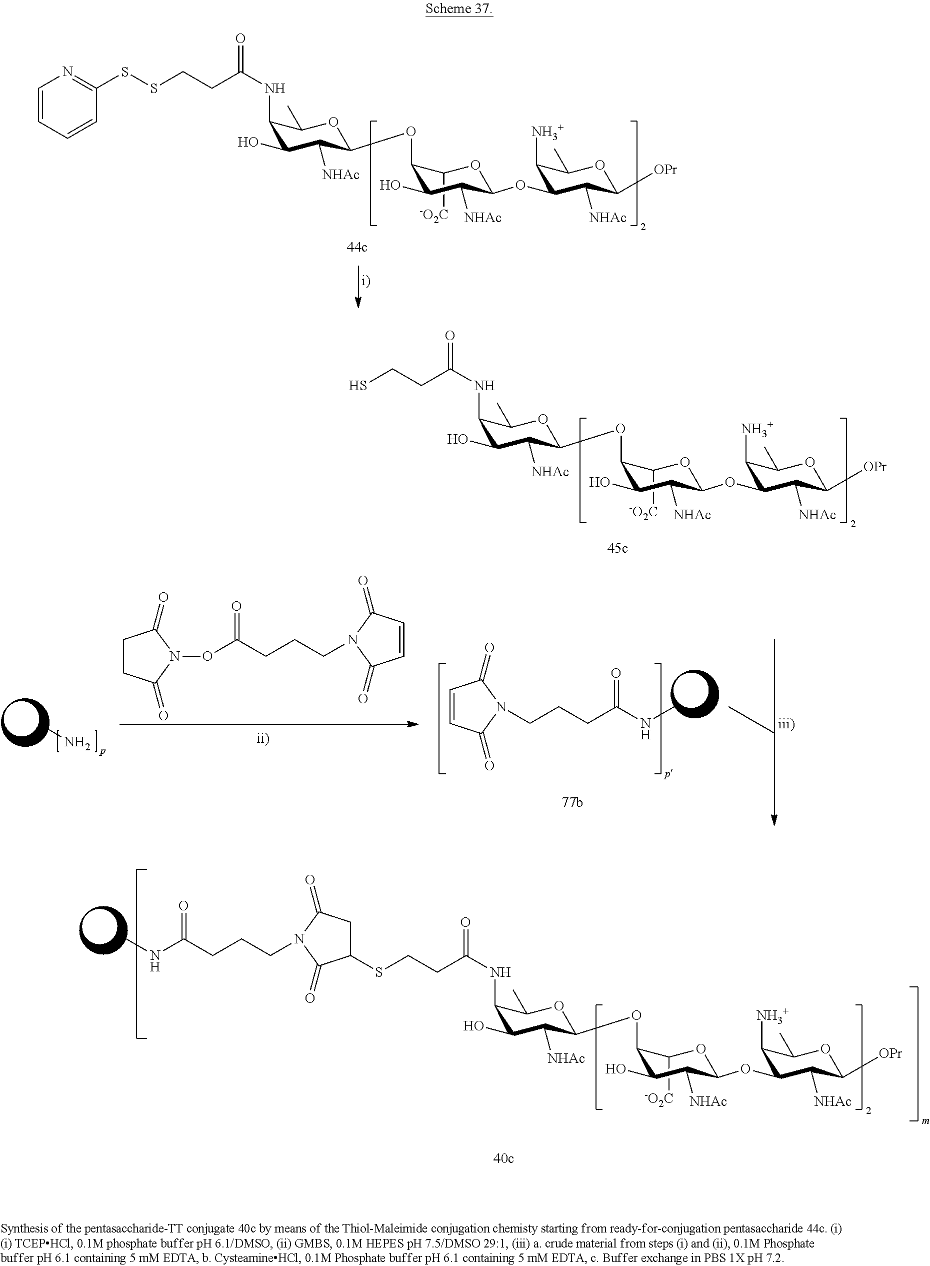

Pentasaccharide-TT conjugates (40c) from thiol-equipped pentasaccharide 44c. SEC purified tetanus toxoid (TT, 150 kD, 270 μL, 12.27 mg/mL) was buffer exchanged with 0.1 M HEPES pH 7.5 by passing through a 30kD centrifugal filter four times and finally concentrated to 210 μL (final concentration of TT: 12.3 mg/mL). A solution of GMBS (744 μg, 2.65 μmol, 155 equiv.) in DMSO (10 μL) was added in two portions to the obtained solution of TT (82 μM, 17.2 nmol). Modification was performed at ambient temperature for 1 h. After this time, the entire volume of intermediate 77b was buffer exchanged with 0.1 M Phosphate buffer pH 6.1 containing 5 mM EDTA, and finally concentrated to 630 μL to reach a final concentration of intermediate of 10.9 mg/mL.

Pentasaccharide 44c (1.2 mg, 962 nmol) was dissolved in 0.1 M phosphate buffer pH 6.1 containing 5 mM EDTA (150 μL). A 209 mM solution of TCEP-HCl in 0.1 M phosphate buffer pH 6.1 (4.5 μL, 275 μg, 960 nmol, 1.0 equiv.) was added and the solution was stirred at rt for 1 h. Monitoring was achieved by RP-HPLC (conditions D) and HRMS revealing the absence of the starting 44c (Rₜ=11.2 min) and the presence of a major product corresponding to the expected thiol 45c. The later had RP-HPLC (215 nm): Rₜ=10.1 min (conditions D). Thiol 45c had HRMS (ESI⁺): m/z [M+H]⁺ calcd for C₄₆H₇₇N₉O₂₃S m/z 1141.4817; found 1141.4815; m/z [M+Na]⁺ calcd for C₄₆H₇₆N₉O₂₃SNa m/z 1163.4636; found 1163.4633. Owing to the presence of the unwanted dimer, the obtained crude was treated with additional TCEP leading to the crude 45c in a total volume of 300 μL.

Different individual portions of various stock solutions of intermediates 77b and 45c, respectively, were used to obtain conjugates with different carbohydrate:protein ratios. The expected amounts of each one of the two solutions were mixed and gently stirred for 3 h at rt. Cysteamine·HCl (0.4 mg, 3.0 μmol, 20 mg/mL in deionised water (20 μL)) was added to all reaction mixtures, facilitating a molar excess of 160 compared to intermediate 77b. After stirring for 30 min at rt, the entire volume of conjugate was buffer exchanged with PBS 1× pH 7.2 and finally harvested. The final conjugates were analyzed by UV (λ=280 nm) and MALDI-MS for conjugation yield and carbohydrate:protein ratio.

Conjugate 40c1: From the stock solution of modified TT (77b, 115 μL, 1.8 mg, 12.0 nmol) and crude thiol 45c (150 μL, 480 nmol, 40 equiv.).

Conjugate 40c2: From the stock solution of modified TT (77b, 105 μL, 1.5 mg, 10.0 nmol) and crude thiol 45c (130 μL, 416 nmol, 42 equiv.).

Moreover, changing TT for CRM 197 successfully provided CRM 197-conjugates, for example tetrasaccharide (AB)₂—CRM conjugates and octasaccharide (AB)₄—CRM conjugates, using analogous procedures.

TABLE 3
S. sonnei (AB)ₙ oligosaccharide (OS)-CRM 197 conjugates
by means of The thiol-Maleimide conjugation chemistry.

| | | | Average | CRM 197 | OS | |
|---|---|---|---|---|---|---|
| | Code in | Conjugate | OS:CRM | conc^{a} | conc | Total |
| | FIG. 8 | number | ratio (m) | mg/mL | μg/mL | volume |
| Ssonnei | Son CA | 92b1 | 13 | 1.9 | 350 | 550 μL |
| SS- | Son DA | 92b2 | 11 | 0.46 | 72 | 420 μL |
| CRM4 | | 92b3 | 7 | 0.46 | 47 | 420 μL |
| Ssonnei | Son EA | 93b1 | 11 | 0.36 | 113 | 550 μL |
| SS- | Son FA | 93b2 | 11.5 | 0.44 | 143 | 450 μL |
| CRM8 | Son GA | 93b3 | 8 | 0.68 | 155 | 470 μL |

### Example 23: Conversion of Ready-for-Conjugation (AB)ₙ Oligosaccharides into (AB)ₙ-CRM Conjugates

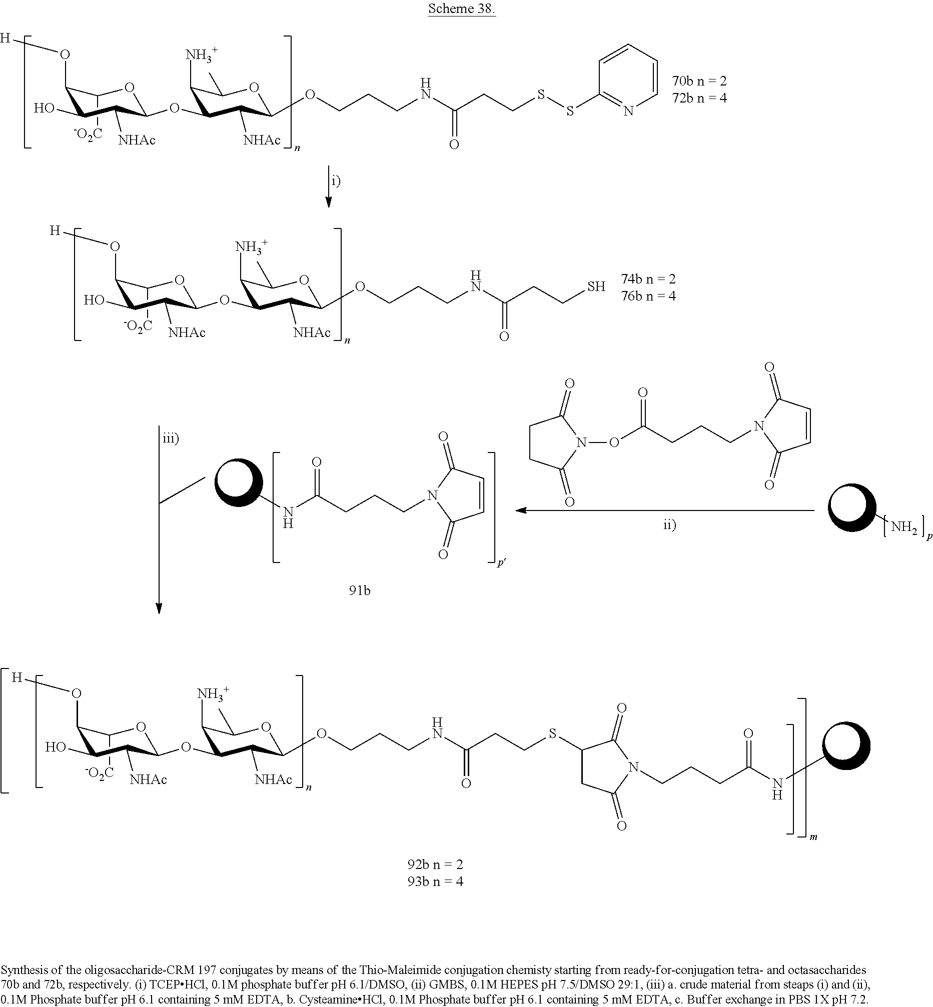

Tetrasaccharide-CRM conjugates (92b) from thiol-equipped tetrasaccharide 74b. Recombinant CRM197 (CRM, 58,443 D, Provepharm Life Solutions (Marseille, France), 1.0 mL, 5.0 mg/mL) was buffer exchanged with 0.1 M HEPES pH 7.5 by passing through a 30kD centrifugal filter four times and finally concentrated to 300 μL (final concentration of CRM: 16.1 mg/mL). A solution of GMBS (2.9 mg, 10.2 μmol, 120 equiv.) in DMSO (15 μL) was added to the obtained solution of CRM (275 μM, 83 nmol). Modification was performed at ambient temperature for 1 h. After this time, the entire volume of intermediate 91b was buffer exchanged with 0.1 M Phosphate buffer pH 6.2 containing 5 mM EDTA, and finally concentrated to 300 μL to reach a final concentration of intermediate 91b of 14.25 mg/mL.

Tetrasaccharide 74b was obtained from 70b (700 μg, 649 nmol) as described above.

A portion of the stock solution of modified CRM (91b, 132 μL, 1.89 mg, 32 nmol) and the total volume of crude tetrasaccharide 74b (649 nmol theo., 20 equiv.) in phosphate buffer pH 6.2 containing 5 mM EDTA was mixed and gently stirred for 20 h at rt. A solution of Cysteamine·HCl (0.4 mg, 20 mg/mL in deionised water (20 μL)) was added facilitating a molar excess of 120 compared to intermediate 91b. After stirring for 30 min at rt, the entire volume of conjugate was purified by SEC eluting with PBS, 0.5 mL/min from an Akta Superdex 200 Increase 10 300 GL column to give three fractions listed as 92b1, 92b2, and 92b3, respectively. The final conjugates were analyzed by UV (λ=280 nm) and MALDI-MS for conjugation yield and carbohydrate:protein ratio.

Octasaccharide-CRM conjugates (93b) from thiol-equipped octasaccharide 76b. Recombinant CRM197 (CRM, 58,443 D, 300 μL, 5.0 mg/mL) was buffer exchanged with 0.1 M HEPES pH 7.5 by passing through a 30kD centrifugal filter four times and finally concentrated to 300 μL (final concentration of CRM: 4.8 mg/mL). A solution of GMBS (863 μg mg, 3.1 μmol, 120 equiv.) in DMSO (5.2 μL) was added to the obtained solution of CRM (82 μM, 25 nmol). Modification was performed at ambient temperature for 1 h. After this time, the entire volume of intermediate 91b was buffer exchanged with 0.1 M Phosphate buffer pH 6.2 containing 5 mM EDTA, and finally concentrated to 260 μL to reach a final concentration of intermediate 91b of 5.1 mg/mL.

Octasaccharide 76b was obtained from 72b (1.5 mg, 796 nmol) as described above.

A portion of the stock solution of modified CRM (91b, 196 μL, 1.0 mg, 17 nmol) and the total volume of crude octasaccharide 74b (780 nmol theo., 46 equiv.) in phosphate buffer pH 6.2 containing 5 mM EDTA was mixed and gently stirred for 3h45 at rt. A solution of Cysteamine·HCl (0.4 mg, 20 mg/mL in deionised water (20 μL)) was added facilitating a molar excess of 120 compared to intermediate 91b. After stirring for 30 min at rt, the entire volume of conjugate was purified by SEC eluting with PBS, 0.5 mL/min from an Akta Superdex 200 Increase 10 300 GL column to give three fractions listed as 93b1, 93b2, and 93b3, respectively. The final conjugates were analyzed by UV (λ=280 nm) and MALDI-MS for conjugation yield and carbohydrate:protein ratio.

It is also noted that the Thiol Maleimide chemistry could be successfully replaced by the Thiol-Bromoacetyl chemistry to yield other conjugates of the invention. Indeed, tetanus toxoid modified with maleimide moieties (77b) has been successfully replaced by tetanus toxoid modified with bromoacetyl groups (94b) and CRM 197 modified with maleimide moieties (91b) has been successfully replaced by CRM 197 modified with bromoacetyl moieties (95b).

TABLE 4
S. sonnei (AB)ₙ oligosaccharide-TT and (AB)ₙ oligosaccharide-
TT conjugates obtained by means of the thiol-bromoacetyl chemistry.

| | | | | | |
|---|---|---|---|---|---|
| Code | | Average | TT | OS | |
| in | Conjugate | OS:TT | conc | conc | Total |
| figure | Nb | ratio (m) | mg/mL | μg/mL | volume |

| | | | | | |
|---|---|---|---|---|---|
| Ssonnei | 96b1 | 10 | 2.13^{a} | 118 | 880 μL |
| SS-TT_{Ac}4 | | | | | |
| Ssonnei | 97b1 | 4.4 | 0.78 | 25 | 650 μL |
| SS-CRM_{Ac}2 | | | | | |
| Ssonnei | 98b1 | 5.5 | 1.47 | 116 | 760 μL |
| SS-CRM_{Ac}4 | | | | | |

^{a}The protein concentration was measured by use of the BCA assay.

### Example 24: Conjugation of Oligosaccharide Haptens onto a Carrier by Means of the Thiol-Bromoacetyl Conjugation Chemistry

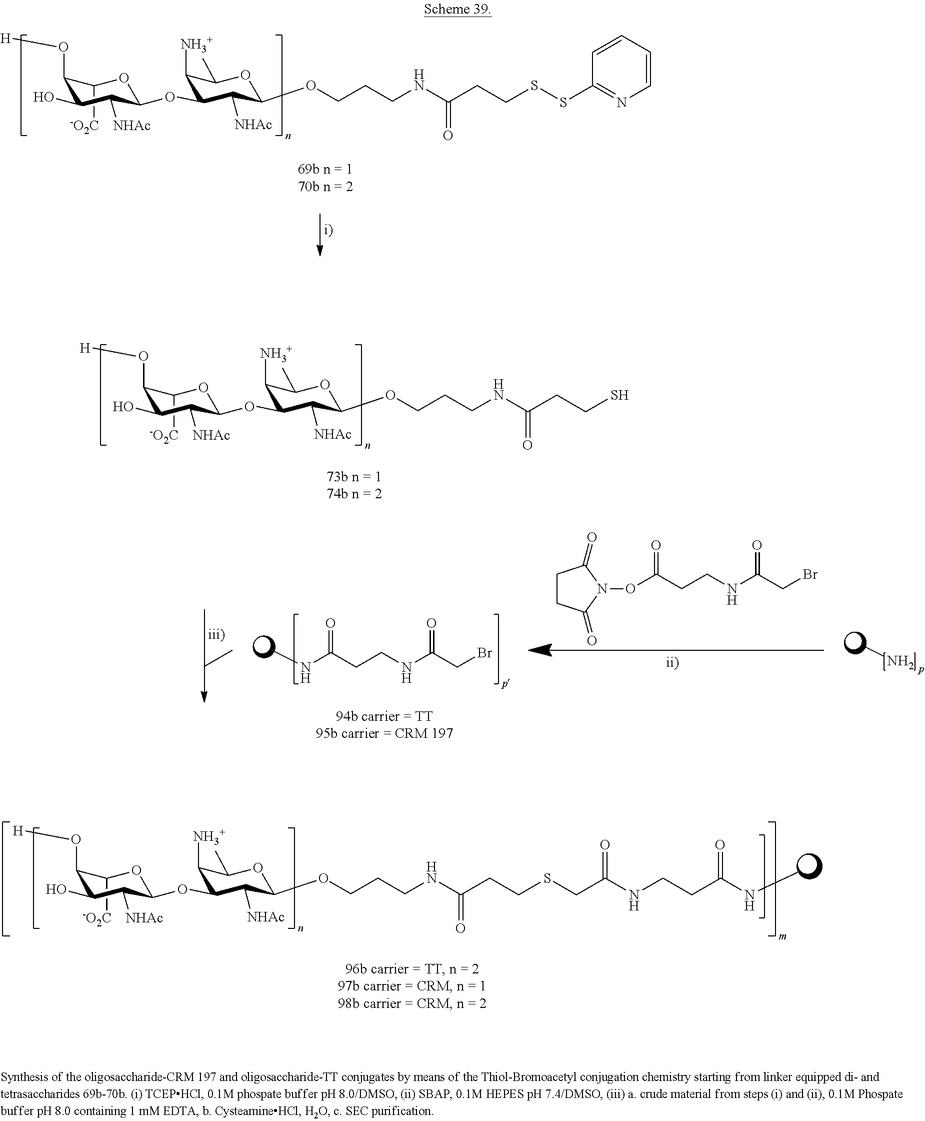

Tetrasaccharide-TT conjugates (96b) from thiol-equipped tetrasaccharide 74b. SEC purified tetanus toxoid (TT, 150 kD, 500 μL, 7.97 mg/mL) was buffer exchanged with 0.1 M HEPES pH 7.4 by passing through a 30kD centrifugal filter four times and finally concentrated to 500 μL (final concentration of TT: 7.66 mg/mL). A solution of SBAP (1.5 mg, 4.9 μmol, 190 equiv.) in DMSO (25 μL) was added to the obtained solution of TT (51 μM, 25 nmol). Modification was performed at ambient temperature for 1 h. After this time, the entire volume of intermediate 94b was buffer exchanged with 0.1 M Phosphate buffer pH 8.0 containing 5 mM EDTA, and finally concentrated to 450 μL to reach a final concentration of intermediate 94b of 7.93 mg/mL.

Tetrasaccharide 70b (2.5 mg, 2.31 μmol) was dissolved in 0.1 M phosphate buffer pH 8.0 (200 μL). A 145 mM solution of TCEP-HCl in DMSO (16 μL, 662 μg, 2.31 μmol, 1.0 equiv.) was added and the solution was stirred at rt for 1 h30. Monitoring was achieved by RP-HPLC as above.

A portion of the crude solution of tetrasaccharide 74b (74 μL, 798 nmol, 60 equiv. theo) was added to a solution of crude 94b (252 μL, 2.0 mg, 13 nmol) and the solution was gently stirred for 20 h at rt. A solution of Cysteamine·HCl (0.4 mg, 20 mg/mL in deionised water (20 μL)) was added, facilitating a molar excess of 190 compared to intermediate 94b. After stirring for 30 min at rt, the entire volume of conjugate was buffer exchanged with PBS 1× pH 7.2 and finally harvested. The final conjugate (96b1) was analyzed by UV (λ=280 nm), BCA and MALDI-MS for conjugation yield and carbohydrate:protein ratio.

Disaccharide-CRM conjugates (97b) from thiol-equipped disaccharide 73b. Recombinant CRM197 (CRM, 58,443 D, 500 μL, 5.0 mg/mL) was buffer exchanged with 0.1 M phosphate buffer pH 7.8 containing 1 mM EDTA by passing through a 30kD centrifugal filter four times and finally concentrated to 450 μL (final concentration of CRM: 5.0 mg/mL). A solution of SBAP (525 μg, 1.7 μmol, 50 equiv.) in DMSO (8.7 μL) was added to a fraction of the obtained solution of CRM (86 μM, 400 μL, 34 nmol). Modification was performed at ambient temperature for 1 h. After this time, the entire volume of intermediate 91b was buffer exchanged with 0.1 M Phosphate buffer pH 7.8 containing 1 mM EDTA, and finally concentrated to 300 μL to reach a final concentration of intermediate 95b of 5.9 mg/mL.

Disaccharide 73b was obtained as above starting from 69b (800 μg, 1.2 μmol) except that phosphate buffer pH 7.8 (200 μL) was used.

Modified CRM197 (95b, 175 μL, 1.03 mg, 18 nmol) was added to the solution of crude thiol 73b (1.2 μmol theo, 60 equiv. theo) and the solution was gently stirred for 20 h at rt. A solution of Cysteamine·HCl (0.4 mg, 20 mg/mL in deionised water (20 μL)) was added facilitating a molar excess of 60 compared to intermediate 95b. After stirring for 30 min at rt, the entire volume of conjugate was purified by SEC eluting from an Akta Superdex 200 Increase 10 300 GL column with PBS×1 at a 0.5 mL/min to give conjugate 97b1. The final conjugate was analyzed by UV (λ=280 nm) and MALDI-MS for conjugation yield and carbohydrate:protein ratio.

Tetrasaccharide-CRM conjugates (98b) from thiol-equipped tetrasaccharide 74b. Recombinant CRM197 (CRM, 58,443 D, 800 μL, 5.0 mg/mL) was buffer exchanged with 0.1 M phosphate buffer pH 7.4 containing 1 mM EDTA by passing through a 30kD centrifugal filter four times and finally concentrated to 450 μL (final concentration of CRM: 8.8 mg/mL). A solution of SBAP (2.0 mg, 6.5 μmol, 95 equiv.) in DMSO (8.7 μL) was added to the obtained solution of CRM (150 μM, 450 μL, 6.8 nmol). Modification was performed at ambient temperature for 1 h. After this time, the entire volume of intermediate 91b was buffer exchanged with 0.1 M Phosphate buffer pH 7.8 containing 1 mM EDTA, and finally concentrated to 300 μL to reach a final concentration of intermediate 95b of 5.9 mg/mL.

Tetrasaccharide 74b was obtained as above starting from 70b (800 μg, 1.2 μmol) except that phosphate buffer pH 7.8 (200 μL) was used.

Modified CRM197 (95b, 175 μL, 1.03 mg, 18 nmol) of the obtained 150 μM solution was added to the solution of crude thiol 74b (1.2 μmol theo, 60 equiv. theo) and the solution was gently stirred for 20 h at rt. A solution of Cysteamine·HCl (0.4 mg, 20 mg/mL in deionised water (20 μL)) was added facilitating a molar excess of 60 compared to intermediate 95b. After stirring for 30 min at rt, the entire volume of conjugate was purified by SEC eluting from an Akta Superdex 200 Increase 10 300 GL column with PBS×1 at a 0.5 mL/min to give conjugate 98b1. The final conjugate was analyzed by UV (λ=280 nm) and MALDI-MS for conjugation yield and carbohydrate:protein ratio.

In addition, aminopropyl glycosides could be successfully converted to oligosaccharides equipped at their reducing end with a linker featuring a single propargyl moiety to yield ready-for-conjugation haptens compatible with conjugation chemistries other than those involving thiol precursors as exemplified for tetrasaccharide 100b.

Alternatively, the aminopropyl glycosides could be successfully converted to oligosaccharides equipped at their reducing end with a linker featuring a single azido moiety to yield ready-for-conjugation haptens compatible with conjugation chemistries other than those involving thiol precursors as exemplified for tetrasaccharide 100b.

### Example 25. Aminopropyl Linker Modification into Conjugation-Ready Oligosaccharides

Chemoselective Introduction of a Propargyl Moiety or an Azido Moiety

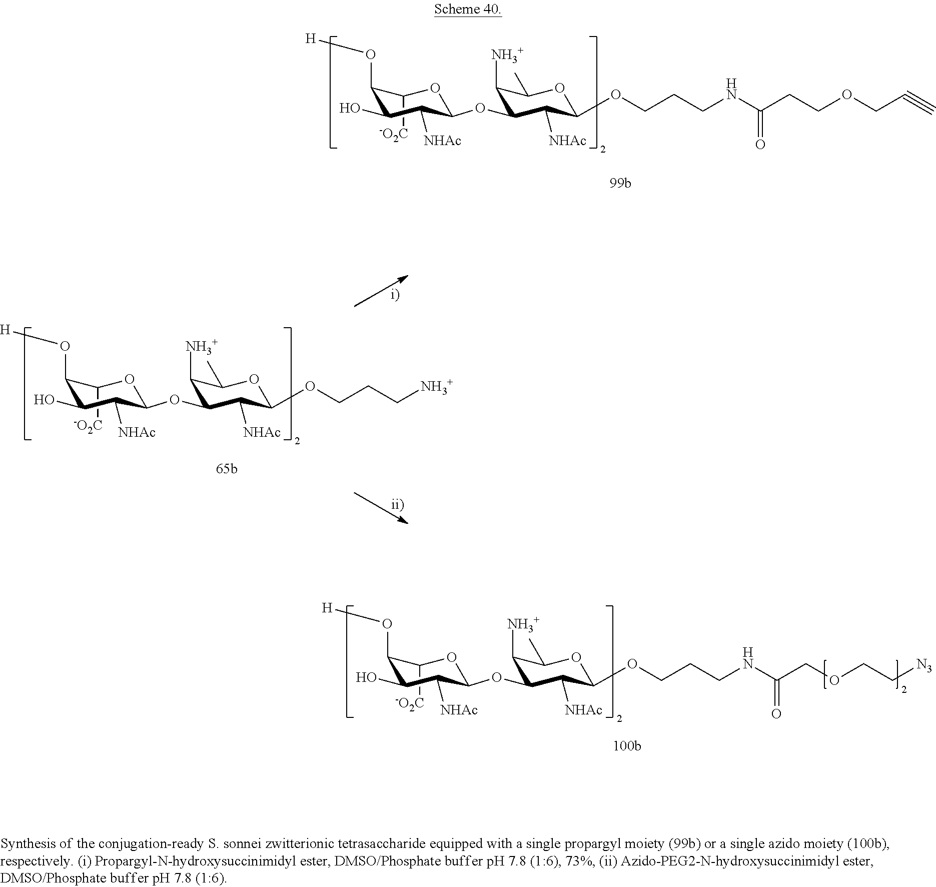

Ready-for-conjugation (AB)₂ tetrasaccharide (99b). Tetrasaccharide 65b (1.1 mg, 1.25 μmol, 1.0 equiv.) was dissolved in 0.1 M phosphate buffer pH 7.8 (300 μL) and stirred vigorously at rt. A solution of propargyl-N-hydroxysuccinimidyl ester (393 μg, 15 μmol, 1.4 equiv.) in DMSO (50 μL) was added. After stirring for 30 min at rt, RP-HPLC monitoring indicated full consumption. The total volume was purified by RP-HPLC. Amide 99b (0.9 mg, 73%) was obtained as a white lyophilized powder. The linker-equipped tetrasaccharide 99b had RP-HPLC (215 nm/ELSD): Rₜ=9.0/9.1 min (conditions D). HRMS (ESI⁺): m/z [M+H]⁺ calcd for C₄₁H₆₆N₇O₂₁S, 992.4306, found 992.4307.

Ready-for-conjugation (AB)₂ tetrasaccharide (100b). Tetrasaccharide 65b (1.4 mg, 1.6 μmol, 1.0 equiv.) was dissolved in 0.1 M phosphate buffer pH 7.8 (300 μL) and stirred vigorously at rt. A solution of azido-PEG2-N-hydroxysuccinimidyl ester (741 μg, 2.6 μmol, 1.6 equiv.) in DMSO (50 μL) was added. After stirring for 3 h at rt, RP-HPLC and LC-MS monitoring indicated the presence of a novel product. The linker-equipped tetrasaccharide 100b had RP-HPLC (215 nm/ELSD): Rₜ=9.0/9.1 min (conditions D). LC-MS (ESI⁺): m/z [M+2H]²⁺ calcd for C₄₁H₇₀N₁₀O₂₂ 527.23, found 527.2; [M+H]⁺ calcd for C₄₁H₆₉N₁₀O₂₂ 1053.45, found 1053.3.

1. A conjugate comprising an oligo- or polysaccharide selected from the group consisting of:
(B)ₓ-(A-B)ₙ-(A)_{y}, and
(A)ₓ-(B-A)ₙ-(B)_{y},
wherein:
x is 0 or 1,
y is 0 or 1,
n ranges from 1 to 50, in particular from 1 or 2 to 10, more particularly from 1 or 2 to 4 or from 3 to 8, n being notably 1, 2, 3, 4, 5, 6, 7, 8, 9 or 10,
A is 4)-α-L-AltpNAcA-(1→,
B is 3)-β-D-FucpNAc4N-(1→,
or a pharmaceutically acceptable salt thereof,
said oligo- or polysaccharide being bound to a carrier, in particular covalently bound to a carrier.

2. The conjugate according to claim 1, wherein the carrier is selected among a protein or a peptide comprising at least one T-helper epitope, or a derivative thereof.

3. The conjugate according to claim 1, wherein the oligo- or polysaccharide is bound to the carrier via a spacer which does not contain any carbohydrate residue.

4. The conjugate according to any one of the claims 1 to 3, characterised in that the ratio of oligo- or polysaccharide to the carrier ranges from 1:1 to 500:1, in particular between 1:1 and 200:1, more particularly, between 1:1 and 30:1, preferably between 5:1 and 25:1, more preferably between 8:1 and 30:1, 40:1 or 50:1, or between 5:1 and 20:1.

5. An immunogenic composition comprising a conjugate according to any one of claims 1 to 4 and a physiologically acceptable vehicle.

6. The immunogenic composition according to claim 5, characterised in that it is formulated for parenteral, oral, intranasal or intradermal administration.

7. The conjugate according to any one of claims 1 to 4 or the immunogenic composition according to claim 5 or 6, for use in vaccination, in particular against S. sonnei infection and/or infection caused by pathogens featuring cross-reactive carbohydrate antigens, for example a Plesiomonas shigelloides infection, notably a P. shigelloides O17 infection.

8. A compound of the following formula:
Q-(B)ₓ-(AB)ₙ-(A)_{y}-OR (IIa) or
Q-(A)ₓ-(BA)ₙ-(B)_{y}—OR (IIb),
wherein:
x is 0 or 1,
y is 0 or 1,
n ranges from 1 to 50,
Q is H or a C₁-C₆ alkyl,
A is 4)-α-L-AltpNAcA-(1→,
B is 3)-β-D-FucpNAc4N-(1→,
R is H, C₁-C₆ alkyl, in particular propyl or methyl, or a group LZ,
L is:
a single bond,
a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, or
a divalent C₁-C₁₂ alkyl, C₂-C₁₂ alkenyl or C₂-C₁₂ alkynyl chain substituted by at least one —OH group, being in particular of the following formula —(CH₂—CH₂—C(OH))_{q}—(CH₂—CH₂)ᵢ, wherein i is 0 or 1 and q ranges from 1 to 10,
—N(R^{a})-D-, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; D is C₁-C₇-alkylene, C₁-C₇-alkoxy, C₁-C₄-alkyl-(OCH₂CH₂)ₚO—C₁-C₄-alkyl, O—C₁-C₄-alkyl-(OCH₂CH₂)ₚO—C₁-C₄-alkyl or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1;
Z is Z₁ or F₁-L₂-Z₂,
Z₁ is a terminal function or group, optionally protected, able to form a covalent bond with a carrier and/or a solid support, or a multivalent scaffold; an anchor; a mono-, oligo- or polysaccharide; or a dye or fluorescent residue.
F₁ is any group enabling to bond the linker L to the linker L₂, F₁ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following: 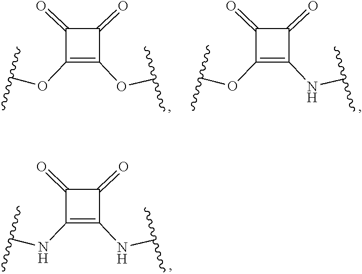
F₁ being more particularly chosen from thio-succinimide, —C(═O)CH₂—NH—, —NH—CH₂—C(═O)—, and triazole functions or groups,
L₂ is a single bond, divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
Z₂ is Z₁ or F₂-L₃-Z₁,
F₂ is any group enabling to bond the linker L to the linker L₃, F₂ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following: 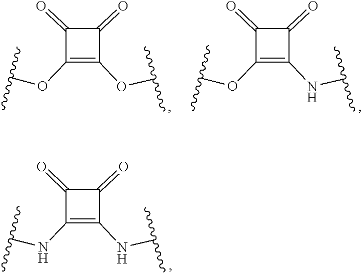
F₂ being more particularly chosen from thio-succinimide, —C(═O)CH₂—NH—, —NH—CH₂—C(═O)—, and triazole functions or groups,
L₃ is single bond, a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
with the proviso that said compound is not H-AB—OPr, H—BA-OPr, H-ABA-OPr, H—BAB—OPr, H-(AB)₂—OPr or H-BA-OMe,
or a pharmaceutically acceptable salt thereof.

9. The compound according to claim 7, selected from the group consisting of: 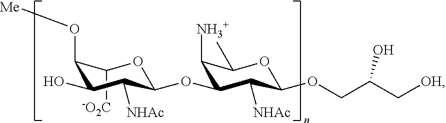
with in particular n=1, 2 or 3, 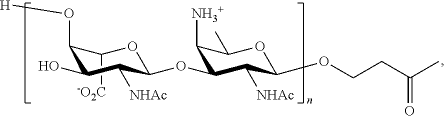
with in particular n=1, 2, 3 or 4, 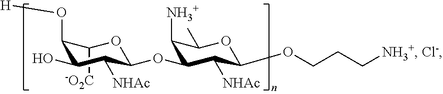
with in particular n=1, 2, 3, 4 or 5, 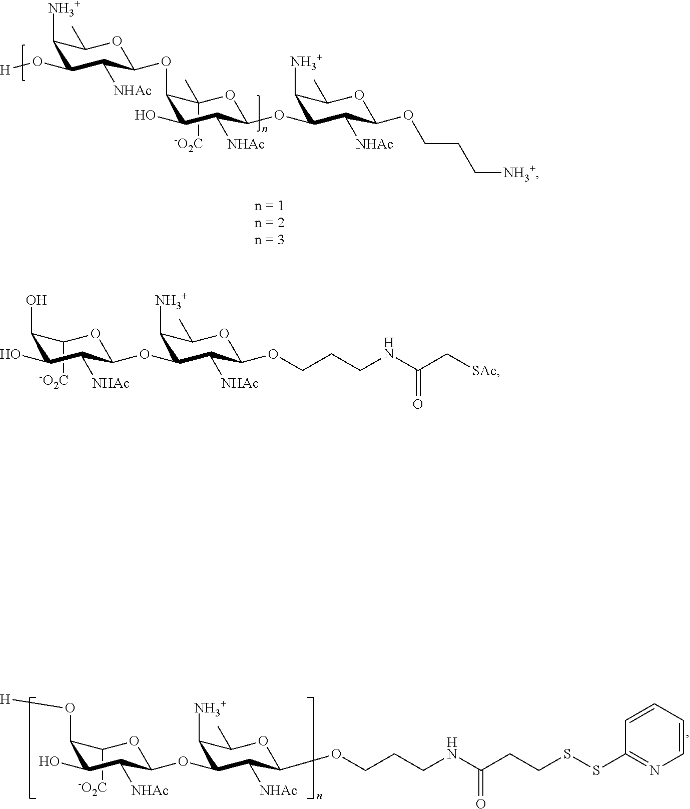
with in particular n=1, 2, 3 or 4, 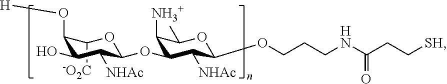
with in particular n=1, 2, 3 or 4, 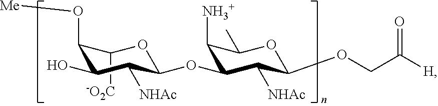
with in particular n=1, 2 or 3,

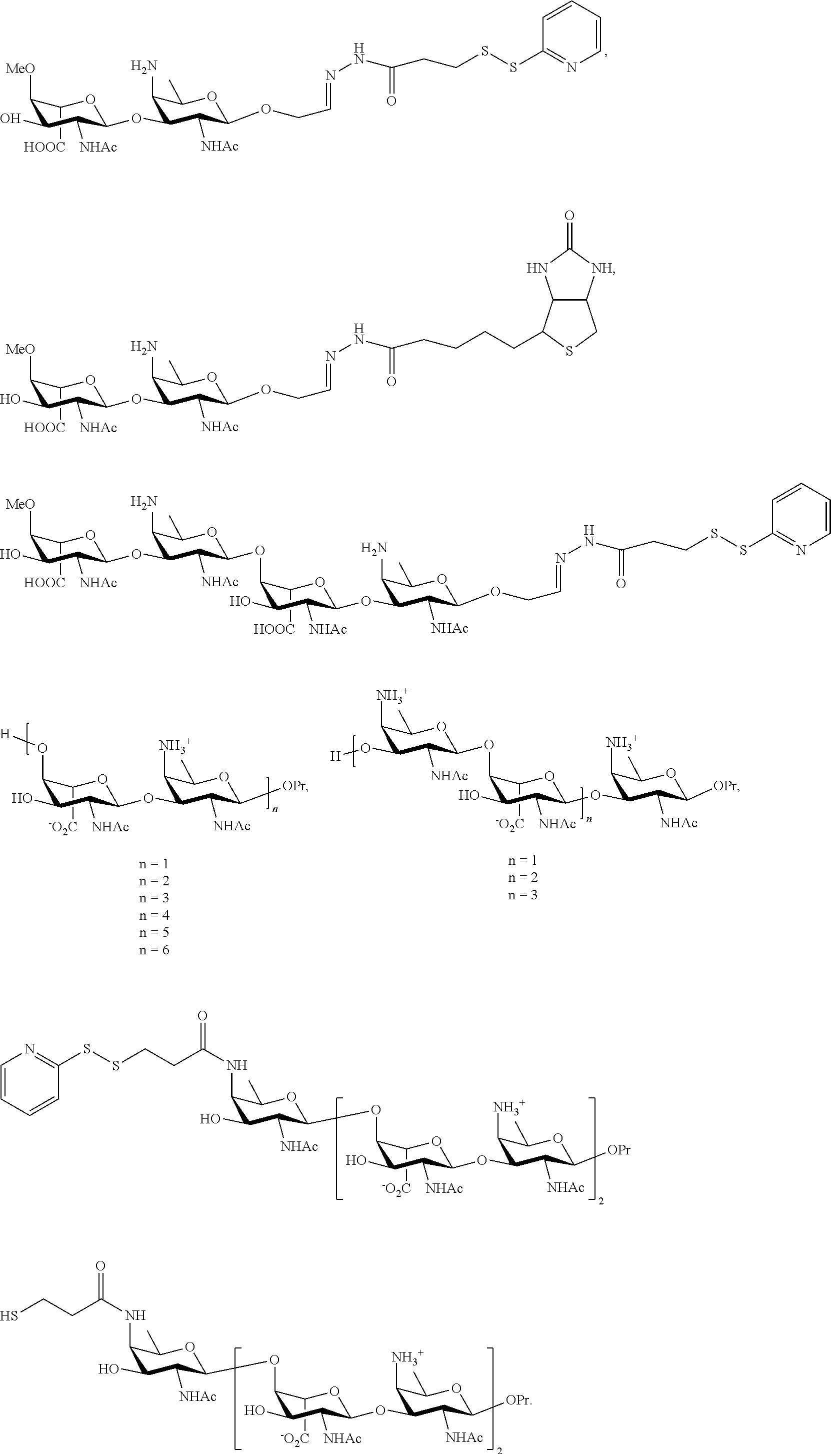

10. Kit for the in vitro diagnostic of S. sonnei infection, wherein said kit comprises a compound according to claim 8 or 9, optionally bound to a label or a solid support.

11. Use of a compound of the following formula (I₀):
T-A′-B′—Y or T-B′-A′-Y (I₀),
wherein:
T is chosen from 2-naphtylmethyl (Nap), para-methoxybenzyl (PMB), 4-bromobenzyl (PBB), benzyloxymethyl acetal (BOM), 2-methoxyethoxymethylether (MEM), methoxypropyl (MOP), tetrahydropyranyl (THP), allyl (All), C₁-C₆ alkyl, or a silyl, T being in particular tert-butyldimethylsilyl (TBS), dimethylhexylsilyl (TDS), triisopropyl silyl (TIPS), or triethylsilyl (TES),
Y is chosen from:
OAll, when T is not All;
Silyl ethers, in particular tert-butyldimethylsilyl ether (OTBS), dimethylhexylsilyl ether (OTDS), triethylsilyl ether (OTES), triisopropyl silyl ether (OTIPS), when T is Nap or PMB;
OPMB, ONap, when OT is a silyl ether or T is All or PBB ether;
p-methoxyphenyl-O (OMP or OPMP); and
SR₄, wherein R₄ is such as the compound is a thioglycoside;
A′ is 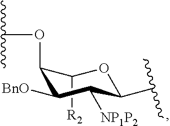
in particular 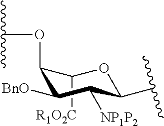
wherein:
P₁ is chosen from TCA, TFA, DCA, CA, Ac, benzyloxycarbamate (Cbz), Trichloroethoxycarbonyl (Troc), and Fmoc, at least one P₁ and P₂ being chosen from TCA, DCA, Ac, Fmoc, Troc, and, when Y is not OAll, OAlloc,
P₂ is H or chosen from Ac, Boc, TFA, benzyloxycarbamate (Cbz), and 2,2,2-trichloroethoxycarbonyl (Troc), P₂ being H when P₁ is not Ac,
or P₁ and P₂ form together a phthalimido or a tetrachlorophthalimido (Cl₄Phth) group,
R₂ is CO₂R₁ or CH₂OR₃, wherein R₃ is Ac, benzoyl (Bz), or R₃ forms together with group T a benzylidene group,
R₁ is chosen from C₁-C₆ alkyl, notably Me or tert-butyl (tBu), Bn and p-methoxybenzyl (PMB) groups, R₁ being in particular Bn,
B′ is 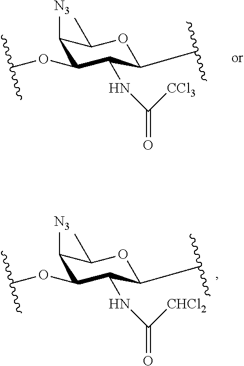
in particular 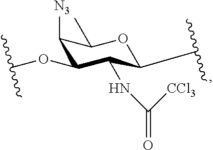
for the preparation of a compound of the following formula (II) Q-(B)ₓ-(AB)ₙ-(A)_{y}-OR (IIa) or Q-(A)ₓ-(BA)ₙ-(B)_{y}—OR (IIb) according to claim 8 or 9.

12. Process of preparation of a compound of the following formula (II):
Q-(B)ₓ-(AB)ₙ-(A)_{y}-OR (IIa) or Q-(A)ₓ-(BA)ₙ-(B)_{y}—OR (IIb), according to claim 8 or 9 said process comprising the following steps:
(i) a step of converting a compound of following formula T-A′-B′—Y, in particular T-A′-B′—OAll, or T-B′-A′-Y (I₀) as defined in claim 11 into a donor compound of following formula T-A′-B′—X or T-B′-A′-X (I_{D}), by intermediately forming the hemiacetal T-A′-B′—OH or T-B′-A′-OH, wherein X represents a leaving group chosen from imidates, for example OPTFA or OTCA, PTFA representing N-phenyltrifluoroacetimidyl and TCA representing trichloroacetimidyl, from o-alkynylbenzoates, and from diphenyl oxosulfoniums, in particular, when Y is All, through metallo-catalyzed deallylation, for example Pd, Ir or Rh, more particularly in presence of H₂-activated Ir-catalyst, then aqueous I₂ or NIS, with optionally a base, in particular an inorganic base, for example NaHCO₃, or by Pd-catalyzed deallylation, in particular in presence of PdCl₂, then aqueous I₂ or NIS, with optionally a base, in particular an inorganic base, for example NaHCO₃, to provide the corresponding hemiacetal T-A′-B′—OH or T-B′-A′-OH and then PTFA-Cl or trichloroacetonitrile,
and/or
(ii) a step of converting a compound of following formula T-A′-B′—Y, in particular T-A′-B′—OAll, or T-B′-A′-Y (I₀) with T being not C₁-C₆ alkyl, into an acceptor compound of following formula H-A′-B′—Y, in particular H-A′-B′—OAll, or H—B′-A′-Y (I_{A}), in particular in presence of 2,3-dichloro-5,6-dicyano-1,4-benzoquinone (DDQ), CAN or an acid when T is Nap or PMB, or in presence of buffered TBAF, for example buffered with AcOH, or Et₃N·3HF, when T is a silyl, or in presence of an organic, inorganic, or Lewis acid, such as AcOH, TsOH, HCl, ZnBr₂ when T is THP, MEM, MOP,
and/or
(iii) a step of obtaining from compound (I_{A}) and/or (I_{D}) a compound Q′-(B′)ₓ-(A′-B′)ₘ (A′)_{y}-Y, in particular Q′-(B′)ₓ-(A′-B′)ₘ-(A′)_{y}-OAll, or Q′-(A′)ₓ-(B′-A′)ₘ-(B′)_{y}—Y (II_{OP}), with m being from 1 to n, and Q′ being T when x is 0 and chosen from T, Bn and acyl groups, for example Lev, ClAc, Fmoc, or Ac when x is 1, in particular in presence of a Lewis acid, for example chosen from TMSOTf, TBSOTf, TfOH, Yb(OTf)₃, Cu(OTf)₂, AgOTf, or boron trifluoride etherate,
(iv) when R is LZ and L is not —N(R^{a})-D-, a step of conjugating compound (II_{OP}) into a compound of following formula Q′-(B′)ₓ-(A′-B′)ₘ-(A′)_{y}-OLZ or Q′-(A′)ₓ-(B′-A′)ₘ-(B′)_{y}-OLZ (II_{CP}), or a compound of following formula Q′-(B′)ₓ-(A′-B′)ₘ-(A′)_{y}-OW or Q′-(A′)ₓ-(B′-A′)ₘ-(B′)_{y}—OW, wherein W is L-F₁′ or L-F₁′_{P}, L being as defined above, F₁′ being a precursor of F₁ as defined above, F₁′_{P} being a protected group F₁′, in particular with one or more benzyl groups,
or when R is LZ and L is —N(R^{a})-D-, a step of preparation of a compound Q′-(B′)ₓ-(A′-B′)ₘ (A′)_{y}-OH or Q′-(A′)ₓ-(B′-A′)ₘ-(B′)_{y}—OH,
(iv′) optionally, when m is not n, a step of converting a compound Q′-(B′)ₓ-(A′-B′)ₘ-(A′)_{y} OLZ or Q′-(A′)ₓ-(B′-A′)ₘ-(B′)_{y}-OLZ (II_{CP}), or a compound of following formula Q′-(B′)ₓ-(A′-B′)ₘ-(A′)_{y}-OW or Q′-(A′)ₓ-(B′-A′)ₘ-(B′)_{y}—OW to a compound Q′-(B′)ₓ-(A′-B′)ₙ-(A′)_{y}-OLZ or Q′-(A′)ₓ-(B′-A′)ₙ-(B′)_{y}-OLZ (II_{CP}), or a compound of following formula Q′-(B′)ₓ-(A′-B′)ₙ-(A′)_{y}-OW or Q′-(A′)ₓ-(B′-A′)ₙ-(B′)_{y}—OW respectively, being noted that when the Q′ group of Q′-(B′)ₓ-(A′-B′)ₘ-(A′)_{y}-OLZ or Q′-(A′)ₓ-(B′-A′)ₘ-(B′)_{y}-OLZ (II_{CP}) or Q′-(B′)ₓ-(A′-B′)ₘ-(A′)_{y}-OW or Q′-(A′)ₓ-(B′-A′)ₘ-(B′)_{y}—OW is not C₁-C₆ alkyl, the Q′ group of Q′-(B′)ₓ-(A′-B′)ₙ-(A′)_{y}-OLZ or Q′-(A′)ₓ-(B′-A′)ₙ-(B′)_{y}-OLZ (II_{CP}) or Q′-(B′)ₓ-(A′-B′)ₙ-(A′)_{y}-OW or Q′-(A′)ₓ-(B′-A′)ₙ-(B′)_{y}—OW can represent C₁-C₆ alkyl,
(v) a step of deprotection of the compound obtained after step (iii) or (iv) to obtain the compound of following formula Q-(B)ₓ-(AB)ₙ-(A)_{y}-OLZ or Q-(A)ₓ-(BA)ₙ-(B)_{y}—OLZ (II) or a compound of following formula Q-(B)ₓ-(AB)ₙ-(A)_{y}-O-L-F₁′ or Q-(A)ₓ-(BA)ₙ-(B)_{y}—O-L-F₁′, or a compound of following formula Q-(B)ₓ-(AB)ₙ-(A)_{y}-OH or Q-(A)ₓ-(BA)ₙ-(B)_{y}—OH, in particular in presence of Pd(OH)₂—C or Pd—C, H₂, for example generated as high-pressure hydrogen with the electrolysis of water, and a base, in particular an inorganic base, for example chosen from NaHCO₃, K₂CO₃, NH₄HCO₃, CaCO₃, MgCO₃, and optionally followed by saponification then in presence of organic/inorganic base for example ethylenediamine, triethylamine, diethylamine, hydoxylamine, NH₂OH or of LiOH/H₂O₂, when R₁ is C₁-C₆ alkyl, notably Me, or before in presence of TBAF or TFA, ZnBr₂, TsOH, when T is a silyl ether, THP, MEM, MOP and/or P₁ is Boc,
(vi) when the compound obtained in step (v) is of formula Q-(B)ₓ-(AB)ₙ-(A)_{y}-O-L-F₁′ or Q-(A)ₓ-(BA)ₙ-(B)_{y}—O-L-F₁′, a step of contacting said compound with
a compound of following formula F₁″-L₂-Z₁, F₁″ being a precursor of F₁ as defined above, L₂ and Z₁ being as defined above, or
a compound of following formula F₁″-L₂-F₂′, F₁″ being a precursor of F₁ as defined above, F₂′ being a precursor of F₂ as defined above, L₂ being as defined above, followed by contacting the obtained compound with a compound of following formula F₂″-L₃-Z₁, wherein F₂″ is a precursor of F₂ as defined above, and L₃ and Z₁ being as defined above,
or when the compound obtained in step (v) is of formula Q-(B)ₓ-(AB)ₙ-(A)_{y}-OH or Q-(A)ₓ-(BA)ₙ-(B)_{y}—OH, a step of contacting said compound with:
a compound of following formula HN(R^{a})-D-Z₁, R^{a}, D and Z₁ being as defined above, or
a compound of following formula HN(R^{a})-D-F₁′, F₁′ being a precursor of F₁ as defined above, followed by contacting the obtained compound with a compound of following formula F₁″-L₂-Z₁, wherein F₁″ is a precursor of F₁ as defined above,
to give the compound of following formula Q-(B)ₓ-(AB)ₙ-(A)_{y}-OLZ or Q-(A)ₓ-(BA)ₙ-(B)_{y}-OLZ (II).

13. Compound of one of the following formulae (III):
Q′-(B′)ₓ-(A′-B′)ₘ-(A′)_{y}-Y or Q′-(B′)ₓ-(A′-B′)ₘ-(A′)_{y}-OLZ,Q′-(B′)ₓ-(A′-B′)ₘ-(A′)_{y}-O-L-F₁′,Q′-(B′)ₓ-(A′-B′)ₘ-(A′)_{y}-O-L-F₁′_{P},Q′-(B′)ₓ-(A′-B′)ₘ-(A′)_{y}-OH,H—(B′)ₓ-(A′-B′)ₘ-(A′)_{y}Y,Q′-(A′)ₓ-(B′-A′)ₘ-(B′)_{y}—Y or Q′-(A′)ₓ-(B′-A′)ₘ-(B′)_{y}-OLZ,Q′-(A′)ₓ-(B′-A′)ₘ-(B′)_{y}—O-L-F₁′,Q′-(A′)ₓ-(B′-A′)ₘ-(B′)_{y}—O-L-F₁′_{P},Q′-(A′)ₓ-(B′-A′)ₘ-(B′)_{y}—OH,H-(A′)ₓ-(B′-A′)ₘ-(B′)_{y}—Y,
wherein:
x is 0 or 1,
Q′ is T when x is 0 and is chosen from T, Bn and acyl groups, for example Lev, ClAc, Fmoc, or Ac when x is 1,
T is chosen from 2-naphtylmethyl (Nap), para-methoxybenzyl (PMB), 4-bromobenzyl (PBB), benzyloxymethyl acetal (BOM), 2-methoxyethoxymethylether (MEM), methoxypropyl (MOP), tetrahydropyranyl (THP), allyl (All), C₁-C₆ alkyl, or a silyl, T being in particular tert-butyldimethylsilyl (TBS), dimethylhexylsilyl (TDS), triisopropyl silyl (TIPS), or triethylsilyl (TES),
A′ is 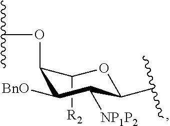
in particular 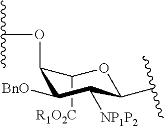
wherein:
P₁ is chosen from TCA, TFA, DCA, CA, Ac, benzyloxycarbamate (Cbz), Trichloroethoxycarbonyl (Troc), and Fmoc, at least one P₁ and P₂ being chosen from TCA, DCA, Ac, Fmoc, Troc, and, when Y is not OAll, OAlloc,
P₂ is H or chosen from Ac, Boc, TFA, benzyloxycarbamate (Cbz), and 2,2,2-trichloroethoxycarbonyl (Troc), P₂ being H when P₁ is not Ac,
or P₁ and P₂ form together a phthalimido or a tetrachlorophthalimido (Cl₄Phth) group,
R₂ is CO₂R₁ or CH₂OR₃, wherein R₃ is Ac, benzoyl (Bz), or R₃ forms with group T a benzylidene group,
R₁ is chosen from C₁-C₆ alkyl, notably Me or tert-butyl (tBu), Bn and p-methoxybenzyl (PMB) groups, R₁ being in particular Bn,
B′ is 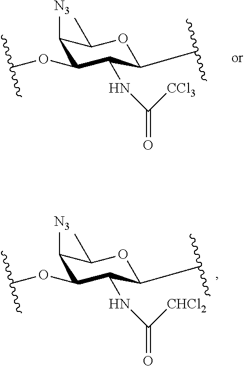
in particular 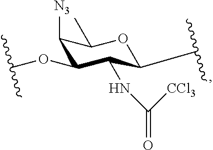
Y is chosen from:
OAll, when T is not All;
Silyl ethers, in particular tert-butyldimethylsilyl ether (OTBS), dimethylhexylsilyl ether (OTDS), triethylsilyl ether (OTES), triisopropyl silyl ether (OTIPS), when T is Nap or PMB;
OPMB, ONap, when OT is a silyl ether or T is All or PBB ether;
p-methoxyphenyl-O (OMP or OPMP); and
SR₄, wherein R₄ is such as the compound is a thioglycoside;
L is:
a single bond,
a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond, or
a divalent C₁-C₁₂ alkyl, C₂-C₁₂ alkenyl or C₂-C₁₂ alkynyl chain substituted by at least one —OH group, being in particular of the following formula —(CH₂—CH₂—C(OH))_{q}—(CH₂—CH₂)ᵢ, wherein i is 0 or 1 and q ranges from 1 to 10,
—N(R^{a})-D-, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; D is C₁-C₇-alkylene, C₁-C₇-alkoxy, C₁-C₄-alkyl-(OCH₂CH₂)ₚO—C₁-C₄-alkyl, O—C₁-C₄-alkyl-(OCH₂CH₂)ₚO—C₁-C₄-alkyl or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1;
Z is Z₁ or F₁-L₂-Z₂,
Z₁ is a terminal function or group, optionally protected, able to form a covalent bond with a carrier and/or a solid support, or a multivalent scaffold; an anchor; a mono-, oligo- or polysaccharide; or a dye or fluorescent residue.
F₁ is any group enabling to bond the linker L to the linker L₂, F₁ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following: 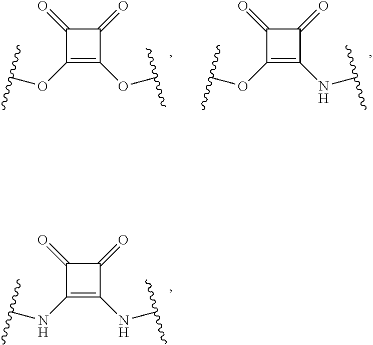 [SMILES: *Oc1c(O*)c(=O)c1=O ; *Nc1c(O*)c(=O)c1=O ; *Nc1c(N*)c(=O)c1=O]
F₁ being more particularly chosen from thio-succinimide, —C(═O)CH₂—NH—, —NH—CH₂—C(═O)—, and triazole functions or groups,
L₂ is a single bond, divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
Z₂ is Z₁ or F₂-L₃-Z₁,
F₂ is any group enabling to bond the linker L to the linker L₃, F₂ being in particular chosen from the —C(═O)—, —C(═O)—C(═O)—, —C(═O)—C(═O)—NH—, —NHC(═O)—C(═O)—, —NHC(═O)—C(═O)—NH—, —C(═O)—C(H)═N—NH—, —NH—C(═O)—C(H)═N—NH—, ester, amide, amine, —CH₂—, ether, thioether, imine, thio-succinimide, oxime, hydrazone, hydrazonamide, —C(═O)CH₂—NH—, —NH—CH₂—C(═O)—, triazole functions or groups, and from the following: 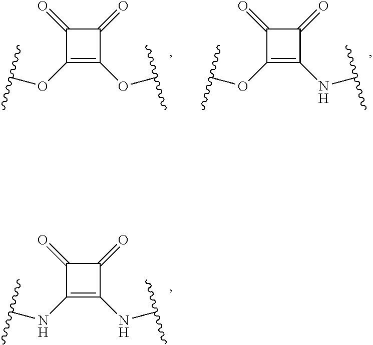 [SMILES: *Oc1c(O*)c(=O)c1=O ; *Nc1c(O*)c(=O)c1=O ; *Nc1c(N*)c(=O)c1=O]
F₂ being more particularly chosen from thio-succinimide, —C(═O)CH₂—NH—, —NH—CH₂—C(═O)—, and triazole functions or groups,
L₃ is single bond, a divalent C₁-C₁₂ alkyl, C₂-C₁₂ alkenyl or C₂-C₁₂ alkynyl chain optionally interrupted by one or more heteroatoms, notably selected from an oxygen atom, a sulphur atom or a nitrogen atom, said nitrogen and sulphur atoms being optionally oxidized, and the nitrogen atom being optionally involved in an acetamide bond,
F₁′ is a precursor of F₁ as defined above,
F₁′_{P} is a protected group F₁′, in particular with one or more benzyl groups,
m is from 1 to n,
n ranges from 1 to 50, in particular from 1 or 2 to 10, more particularly from 1 or 2 to 4 or from 3 to 8, n being notably 1, 2, 3, 4, 5, 6, 7, 8, 9 or 10,
with the proviso that:
when the compound is H—(B′)ₓ-(A′-B′)ₘ(A′)_{y}-Y, in particular H—(B′)ₓ-(A′-B′)ₘ-(A′)_{y}-OAll, and x=y=0, then m ranges from 3 to 50, in particular from 4 to 12;
when the compound is Q′-(B′)ₓ-(A′-B′)ₘ-(A′)_{y}-Y or Q′-(A′)ₓ-(B′-A′)ₘ(B′)_{y}—Y, in particular Q′-(B′)ₓ-(A′-B′)ₘ-(A′)_{y}-OAll or Q′-(A′)ₓ-(B′-A′)ₘ(B′)_{y}-OAll, and x+y=1, then m ranges from 2 to 50, in particular from 3, 4 or 5 to 12 or 50.

14. Compound of one of the following formulae: 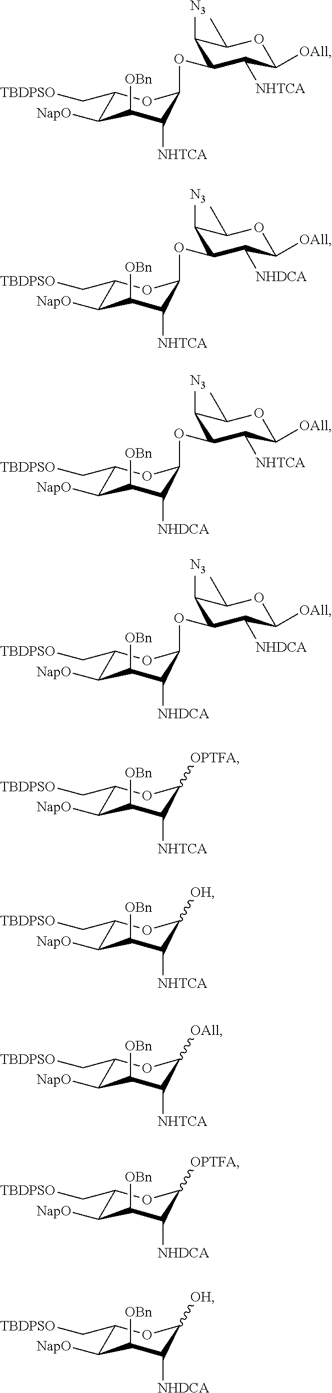 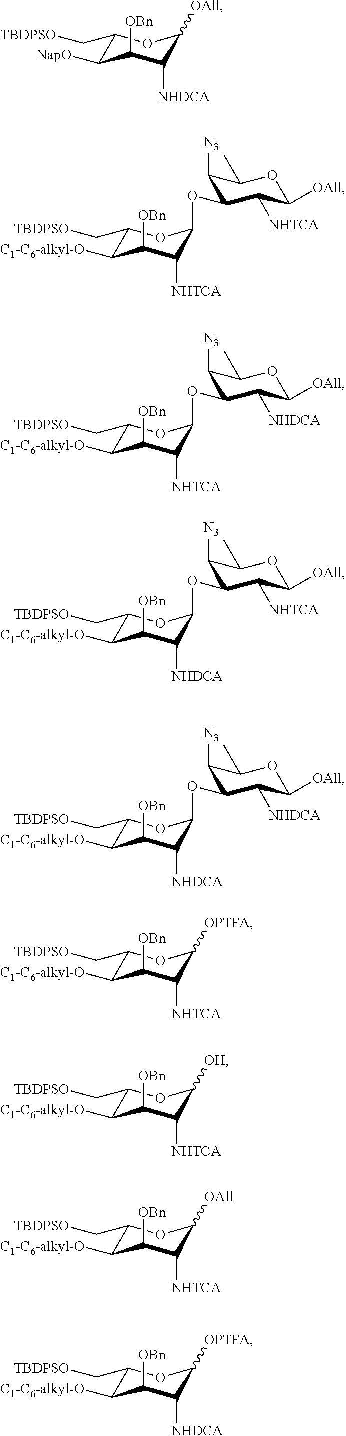 [chemical structure]
